SEQUENCE LISTING (1238 sequences)

SEQ ID NO: 1 - protein, 13 aa, synthetic construct
TRSSGSIASNYVQ

SEQ ID NO: 2 - protein, 11 aa, synthetic construct
SGDKLGNKYAC

SEQ ID NO: 3 - protein, 11 aa, synthetic construct
SGDKLRDKYAC

SEQ ID NO: 4 - protein, 11 aa, synthetic construct
SGDKLGDKYAG

SEQ ID NO: 5 - protein, 13 aa, synthetic construct
TRSSGSIASNYVQ

SEQ ID NO: 6 - protein, 11 aa, synthetic construct
SGDALPKKYAY

SEQ ID NO: 7 - protein, 13 aa, synthetic construct
TRSSGSIASNYVQ

SEQ ID NO: 8 - protein, 11 aa, synthetic construct
SGDALPKKYAY

SEQ ID NO: 9 - protein, 11 aa, synthetic construct
SGDALPKKYAY

SEQ ID NO: 10 - protein, 13 aa, synthetic construct
TRSSGSIASNYVQ

SEQ ID NO: 11 - protein, 13 aa, synthetic construct
TRSSGSIASNYVQ

SEQ ID NO: 12 - protein, 13 aa, synthetic construct
TRSNGSITSNYVQ

SEQ ID NO: 13 - protein, 11 aa, synthetic construct
RASQSISNWLA

SEQ ID NO: 14 - protein, 13 aa, synthetic construct
TRSSGSIASNYVQ

SEQ ID NO: 15 - protein, 13 aa, synthetic construct
TRSSGSIASNYVQ

SEQ ID NO: 16 - protein, 11 aa, synthetic construct
RASQSIGSWLA

SEQ ID NO: 17 - protein, 13 aa, synthetic construct
TRSSGSIASKYVQ

SEQ ID NO: 18 - protein, 16 aa, synthetic construct
RSSQSLVHSDGNTYLN

SEQ ID NO: 19 - protein, 13 aa, synthetic construct
TRSSGSIASNYVQ

SEQ ID NO: 20 - protein, 13 aa, synthetic construct
TRSSGSIASNYVQ

SEQ ID NO: 21 - protein, 11 aa, synthetic construct
SGDALPKKYAY

SEQ ID NO: 22 - protein, 11 aa, synthetic construct
SGDALPKQYTY

SEQ ID NO: 23 - protein, 13 aa, synthetic construct
TRSSGSIASNYVQ

SEQ ID NO: 24 - protein, 13 aa, synthetic construct
TRSSGSIASNYVQ

SEQ ID NO: 25 - protein, 13 aa, synthetic construct
TRSSGSIASNYVQ

SEQ ID NO: 26 - protein, 13 aa, synthetic construct
TRSSGSIASNYVQ

SEQ ID NO: 27 - protein, 14 aa, synthetic construct
GLNSGSVSTSYYPS

SEQ ID NO: 28 - protein, 13 aa, synthetic construct
TRSSGSIASNYVQ

SEQ ID NO: 29 - protein, 13 aa, synthetic construct
SGSSSNIGSNYVY

SEQ ID NO: 30 - protein, 13 aa, synthetic construct
TRSSGSIASNYVQ

SEQ ID NO: 31 - protein, 16 aa, synthetic construct
RSSQSLLHSNGYNYLD

SEQ ID NO: 32 - protein, 11 aa, synthetic construct
SGDKLGYKYAS

SEQ ID NO: 33 - protein, 13 aa, synthetic construct
TRSSGSIASNYVQ

SEQ ID NO: 34 - protein, 13 aa, synthetic construct
TRSSGSIASNYVQ

SEQ ID NO: 35 - protein, 11 aa, synthetic construct
SGDALPKKYAY

SEQ ID NO: 36 - protein, 11 aa, synthetic construct
RASQSISSWLA

SEQ ID NO: 37 - protein, 11 aa, synthetic construct
RASQSISSWLA

SEQ ID NO: 38 - protein, 14 aa, synthetic construct
TGSSSNIGAGYDVH

SEQ ID NO: 39 - protein, 16 aa, synthetic construct
RSSQSLVYSDGNTYLT

SEQ ID NO: 40 - protein, 13 aa, synthetic construct
SGSSSNIGSNTVN

SEQ ID NO: 41 - protein, 11 aa, synthetic construct
SGDALPKKYAY

SEQ ID NO: 42 - protein, 11 aa, synthetic construct
SGDALPKQYTY

SEQ ID NO: 43 - protein, 14 aa, synthetic construct
TGTSSDVGSYNLVS

SEQ ID NO: 44 - protein, 14 aa, synthetic construct
MLSSGFSVGDFWIR

SEQ ID NO: 45 - protein, 11 aa, synthetic construct
RASQSISSWLA

SEQ ID NO: 46 - protein, 13 aa, synthetic construct
TRSSGSIASNYVQ

SEQ ID NO: 47 - protein, 13 aa, synthetic construct
TRSSGSIASKYVQ

SEQ ID NO: 48 - protein, 13 aa, synthetic construct
TRSSGSIASNYVQ

SEQ ID NO: 49 - protein, 13 aa, synthetic construct
TRSSGSIASNYVQ

SEQ ID NO: 50 - protein, 13 aa, synthetic construct
TRSSGSIASNYVQ

SEQ ID NO: 51 - protein, 13 aa, synthetic construct
TRSSGSIASNYVQ

SEQ ID NO: 52 - protein, 11 aa, synthetic construct
SGDALPKKYAY

SEQ ID NO: 53 - protein, 13 aa, synthetic construct
TRSSGSIASNYVQ

SEQ ID NO: 54 - protein, 13 aa, synthetic construct
TRSSGSIASYYVQ

SEQ ID NO: 55 - protein, 13 aa, synthetic construct
TRSSGSIASNYVQ

SEQ ID NO: 56 - protein, 13 aa, synthetic construct
TRSSGSIASNYVQ

SEQ ID NO: 57 - protein, 14 aa, synthetic construct
MLSSGFSVGDFWIR

SEQ ID NO: 58 - protein, 13 aa, synthetic construct
TRSSGSIASNFVQ

SEQ ID NO: 59 - protein, 13 aa, synthetic construct
TRSSGSIASNYVQ

SEQ ID NO: 60 - protein, 13 aa, synthetic construct
TRSSGSIASNYVQ

SEQ ID NO: 61 - protein, 14 aa, synthetic construct
ASSTGAVTSGYYPN

SEQ ID NO: 62 - protein, 13 aa, synthetic construct
TRSSGSIASNYVQ

SEQ ID NO: 63 - protein, 13 aa, synthetic construct
TRTSGSIASNYVQ

SEQ ID NO: 64 - protein, 13 aa, synthetic construct
TRSSGSIASNYVQ

SEQ ID NO: 65 - protein, 13 aa, synthetic construct
SGSSSNIGSNYVY

SEQ ID NO: 66 - protein, 13 aa, synthetic construct
TRSSGSIASNYVQ

SEQ ID NO: 67 - protein, 13 aa, synthetic construct
TRSSGSIASNYVQ

SEQ ID NO: 68 - protein, 11 aa, synthetic construct
SGDALPKQYAY

SEQ ID NO: 69 - protein, 13 aa, synthetic construct
TRSSGSIASNYVQ

SEQ ID NO: 70 - protein, 13 aa, synthetic construct
TRSSGSIASNYVQ

SEQ ID NO: 71 - protein, 13 aa, synthetic construct
TRSSGSIASNYVQ

SEQ ID NO: 72 - protein, 13 aa, synthetic construct
TRSSGSIASNYVQ

SEQ ID NO: 73 - protein, 13 aa, synthetic construct
TRSSGSIASNYVQ

SEQ ID NO: 74 - protein, 11 aa, synthetic construct
SGDALPKKYAY

SEQ ID NO: 75 - protein, 11 aa, synthetic construct
SGDALPKKYAY

SEQ ID NO: 76 - protein, 11 aa, synthetic construct
SGDALPKKYAY

SEQ ID NO: 77 - protein, 14 aa, synthetic construct
TGSSSNIGAGYDVH

SEQ ID NO: 78 - protein, 13 aa, synthetic construct
TRSSGSIASNYVQ

SEQ ID NO: 79 - protein, 14 aa, synthetic construct
GLSSGSVSTSYYPS

SEQ ID NO: 80 - protein, 11 aa, synthetic construct
SGDALPKKYAY

SEQ ID NO: 81 - protein, 13 aa, synthetic construct
TRSSGSIASNYVQ

SEQ ID NO: 82 - protein, 11 aa, synthetic construct
SGDALPKKYAY

SEQ ID NO: 83 - protein, 13 aa, synthetic construct
TRSSGSIASNYVQ

SEQ ID NO: 84 - protein, 13 aa, synthetic construct
TRSSGSIASNYVQ

SEQ ID NO: 85 - protein, 13 aa, synthetic construct
TRSSGSIASNYVQ

SEQ ID NO: 86 - protein, 13 aa, synthetic construct
TRNSGSIASNYVQ

SEQ ID NO: 87 - protein, 13 aa, synthetic construct
TRSSGSIASNYVQ

SEQ ID NO: 88 - protein, 11 aa, synthetic construct
RASQSISSWLA

SEQ ID NO: 89 - protein, 12 aa, synthetic construct
RASQSVSSSYLA

SEQ ID NO: 90 - protein, 13 aa, synthetic construct
TRSSGSIASNYVQ

SEQ ID NO: 91 - protein, 13 aa, synthetic construct
TRSSGSIASNYVQ

SEQ ID NO: 92 - protein, 11 aa, synthetic construct
SGDKLGNKYAC

SEQ ID NO: 93 - protein, 13 aa, synthetic construct
TRSSGSIASNYVQ

SEQ ID NO: 94 - protein, 13 aa, synthetic construct
TRSSGSIASNYVQ

SEQ ID NO: 95 - protein, 13 aa, synthetic construct
TRSSGSIASNYVQ

SEQ ID NO: 96 - protein, 13 aa, synthetic construct
TRSSGSIASNYVQ

SEQ ID NO: 97 - protein, 11 aa, synthetic construct
SGDKLGDKYAC

SEQ ID NO: 98 - protein, 13 aa, synthetic construct
TRSSGSIASNYVQ

SEQ ID NO: 99 - protein, 13 aa, synthetic construct
TRSSGSIASNYVQ

SEQ ID NO: 100 - protein, 11 aa, synthetic construct
SGDKLGNKYAC

SEQ ID NO: 101 - protein, 13 aa, synthetic construct
TRSSGSIASNYVQ

SEQ ID NO: 102 - protein, 13 aa, synthetic construct
TRSSGSIASNYVQ

SEQ ID NO: 103 - protein, 13 aa, synthetic construct
TRSSGSIASNYVQ

SEQ ID NO: 104 - protein, 13 aa, synthetic construct
TRSSGSIASNYVQ

SEQ ID NO: 105 - protein, 13 aa, synthetic construct
TRSSGSIASNYVQ

SEQ ID NO: 106 - protein, 13 aa, synthetic construct
TRSSGSIASNYVQ

SEQ ID NO: 107 - protein, 13 aa, synthetic construct
SGSSSNIGSNTVN

SEQ ID NO: 108 - protein, 13 aa, synthetic construct
TRSSGSIASNYVQ

SEQ ID NO: 109 - protein, 13 aa, synthetic construct
TRSSGSIASNYVQ

SEQ ID NO: 110 - protein, 13 aa, synthetic construct
TRSSGSIASNYVQ

SEQ ID NO: 111 - protein, 12 aa, synthetic construct
RASQSVSSSYLA

SEQ ID NO: 112 - protein, 14 aa, synthetic construct
GLSSGSVSTSYYPS

SEQ ID NO: 113 - protein, 14 aa, synthetic construct
GLSSGSVSTSYYPS

SEQ ID NO: 114 - protein, 7 aa, synthetic construct
EDNQRPS

SEQ ID NO: 115 - protein, 7 aa, synthetic construct
QDSKRPS

SEQ ID NO: 116 - protein, 7 aa, synthetic construct
QDSKRPS

SEQ ID NO: 117 - protein, 7 aa, synthetic construct
QDSKRPS

SEQ ID NO: 118 - protein, 7 aa, synthetic construct
EDNQRPS

SEQ ID NO: 119 - protein, 7 aa, synthetic construct
EDSKRPS

SEQ ID NO: 120 - protein, 7 aa, synthetic construct
EDNQRPS

SEQ ID NO: 121 - protein, 7 aa, synthetic construct
EDSKRPS

SEQ ID NO: 122 - protein, 7 aa, synthetic construct
EDSKRPS

SEQ ID NO: 123 - protein, 7 aa, synthetic construct
EDNQRPS

SEQ ID NO: 124 - protein, 7 aa, synthetic construct
EDNQRPS

SEQ ID NO: 125 - protein, 7 aa, synthetic construct
EDNQRPS

SEQ ID NO: 126 - protein, 7 aa, synthetic construct
KASSLES

SEQ ID NO: 127 - protein, 7 aa, synthetic construct
EDNQRPS

SEQ ID NO: 128 - protein, 7 aa, synthetic construct
EDNQRPS

SEQ ID NO: 129 - protein, 7 aa, synthetic construct
KASSLES

SEQ ID NO: 130 - protein, 7 aa, synthetic construct
EDNQRPS

SEQ ID NO: 131 - protein, 7 aa, synthetic construct
KISDRFS

SEQ ID NO: 132 - protein, 7 aa, synthetic construct
EDNQRPS

SEQ ID NO: 133 - protein, 7 aa, synthetic construct
EDNQRPS

SEQ ID NO: 134 - protein, 7 aa, synthetic construct
EDSKRPS

SEQ ID NO: 135 - protein, 7 aa, synthetic construct
KDSERPS

SEQ ID NO: 136 - protein, 7 aa, synthetic construct
EDNHRPS

SEQ ID NO: 137 - protein, 7 aa, synthetic construct
EDNQRPS

SEQ ID NO: 138 - protein, 7 aa, synthetic construct
EDNHRPS

SEQ ID NO: 139 - protein, 7 aa, synthetic construct
EDNHRPS

SEQ ID NO: 140 - protein, 7 aa, synthetic construct
STNTRSS

SEQ ID NO: 141 - protein, 7 aa, synthetic construct
EDNQRPS

SEQ ID NO: 142 - protein, 7 aa, synthetic construct
SNNQRPS

SEQ ID NO: 143 - protein, 7 aa, synthetic construct
EDDQRPS

SEQ ID NO: 144 - protein, 7 aa, synthetic construct
LGSNRAS

SEQ ID NO: 145 - protein, 7 aa, synthetic construct
QDSKRPS

SEQ ID NO: 146 - protein, 7 aa, synthetic construct
EDNQRPS

SEQ ID NO: 147 - protein, 7 aa, synthetic construct
EDNQRPS

SEQ ID NO: 148 - protein, 7 aa, synthetic construct
EDSKRPS

SEQ ID NO: 149 - protein, 7 aa, synthetic construct
KASSLES

SEQ ID NO: 150 - protein, 7 aa, synthetic construct
KASSLES

SEQ ID NO: 151 - protein, 7 aa, synthetic construct
GNSNRPS

SEQ ID NO: 152 - protein, 7 aa, synthetic construct
KVSNRDS

SEQ ID NO: 153 - protein, 7 aa, synthetic construct
SNNQRPS

SEQ ID NO: 154 - protein, 7 aa, synthetic construct
EDSKRPS

SEQ ID NO: 155 - protein, 7 aa, synthetic construct
KDSERPS

SEQ ID NO: 156 - protein, 7 aa, synthetic construct
EDIRRPS

SEQ ID NO: 157 - protein, 7 aa, synthetic construct
YHSDSNK

SEQ ID NO: 158 - protein, 7 aa, synthetic construct
KASSLES

SEQ ID NO: 159 - protein, 7 aa, synthetic construct
EDNQRPS

SEQ ID NO: 160 - protein, 7 aa, synthetic construct
EDNQRPS

SEQ ID NO: 161 - protein, 7 aa, synthetic construct
EDNQRPS

SEQ ID NO: 162 - protein, 7 aa, synthetic construct
EDNQRSS

SEQ ID NO: 163 - protein, 7 aa, synthetic construct
EDNQRPS

SEQ ID NO: 164 - protein, 7 aa, synthetic construct
EDNQRPS

SEQ ID NO: 165 - protein, 7 aa, synthetic construct
EDSKRPS

SEQ ID NO: 166 - protein, 7 aa, synthetic construct
EDNQRPS

SEQ ID NO: 167 - protein, 7 aa, synthetic construct
EDNQRPS

SEQ ID NO: 168 - protein, 7 aa, synthetic construct
EDNQRPS

SEQ ID NO: 169 - protein, 7 aa, synthetic construct
EDNQRPS

SEQ ID NO: 170 - protein, 7 aa, synthetic construct
YHSDSNK

SEQ ID NO: 171 - protein, 7 aa, synthetic construct
EDNQRPS

SEQ ID NO: 172 - protein, 7 aa, synthetic construct
EDNQRPS

SEQ ID NO: 173 - protein, 7 aa, synthetic construct
EDNQRPS

SEQ ID NO: 174 - protein, 7 aa, synthetic construct
STSNKHS

SEQ ID NO: 175 - protein, 7 aa, synthetic construct
EDNQRPS

SEQ ID NO: 176 - protein, 7 aa, synthetic construct
EDNQRPS

SEQ ID NO: 177 - protein, 7 aa, synthetic construct
EDNQRPS

SEQ ID NO: 178 - protein, 7 aa, synthetic construct
SNNQRPS

SEQ ID NO: 179 - protein, 7 aa, synthetic construct
EDNQRPS

SEQ ID NO: 180 - protein, 7 aa, synthetic construct
EDNQRPS

SEQ ID NO: 181 - protein, 7 aa, synthetic construct
KDSERPS

SEQ ID NO: 182 - protein, 7 aa, synthetic construct
EDNQRPS

SEQ ID NO: 183 - protein, 7 aa, synthetic construct
EDNQRPS

SEQ ID NO: 184 - protein, 7 aa, synthetic construct
EDNQRPS

SEQ ID NO: 185 - protein, 7 aa, synthetic construct
EDNQRPS

SEQ ID NO: 186 - protein, 7 aa, synthetic construct
EDNRRPS

SEQ ID NO: 187 - protein, 7 aa, synthetic construct
EDSKRPS

SEQ ID NO: 188 - protein, 7 aa, synthetic construct
EDSKRPS

SEQ ID NO: 189 - protein, 7 aa, synthetic construct
EDSKRPS

SEQ ID NO: 190 - protein, 7 aa, synthetic construct
GNSNRPS

SEQ ID NO: 191 - protein, 7 aa, synthetic construct
EDNQRPS

SEQ ID NO: 192 - protein, 7 aa, synthetic construct
STNTRSS

SEQ ID NO: 193 - protein, 7 aa, synthetic construct
EDSKRPS

SEQ ID NO: 194 - protein, 7 aa, synthetic construct
EDNQRPS

SEQ ID NO: 195 - protein, 7 aa, synthetic construct
EDSKRPS

SEQ ID NO: 196 - protein, 7 aa, synthetic construct
EDNQRPS

SEQ ID NO: 197 - protein, 7 aa, synthetic construct
EDTQRPS

SEQ ID NO: 198 - protein, 7 aa, synthetic construct
EDNHRPS

SEQ ID NO: 199 - protein, 7 aa, synthetic construct
EDKQRPS

SEQ ID NO: 200 - protein, 7 aa, synthetic construct
EDNQRPS

SEQ ID NO: 201 - protein, 7 aa, synthetic construct
KASSLES

SEQ ID NO: 202 - protein, 7 aa, synthetic construct
GASSRAT

SEQ ID NO: 203 - protein, 7 aa, synthetic construct
EDNQRPS

SEQ ID NO: 204 - protein, 7 aa, synthetic construct
EDNQRPS

SEQ ID NO: 205 - protein, 7 aa, synthetic construct
QDNKRPS

SEQ ID NO: 206 - protein, 7 aa, synthetic construct
EDNQRPS

SEQ ID NO: 207 - protein, 7 aa, synthetic construct
EDNQRPS

SEQ ID NO: 208 - protein, 7 aa, synthetic construct
EDNQRPS

SEQ ID NO: 209 - protein, 7 aa, synthetic construct
EDNQRPS

SEQ ID NO: 210 - protein, 7 aa, synthetic construct
QDSKRPS

SEQ ID NO: 211 - protein, 7 aa, synthetic construct
EDNQRPS

SEQ ID NO: 212 - protein, 7 aa, synthetic construct
EDNQRPS

SEQ ID NO: 213 - protein, 7 aa, synthetic construct
QDSKRPS

SEQ ID NO: 214 - protein, 7 aa, synthetic construct
EDNQRPS

SEQ ID NO: 215 - protein, 7 aa, synthetic construct
EDNQRPS

SEQ ID NO: 216 - protein, 7 aa, synthetic construct
EDNQRPS

SEQ ID NO: 217 - protein, 7 aa, synthetic construct
EDNQRPS

SEQ ID NO: 218 - protein, 7 aa, synthetic construct
EDNQRPS

SEQ ID NO: 219 - protein, 7 aa, synthetic construct
EDNQRPS

SEQ ID NO: 220 - protein, 7 aa, synthetic construct
SNNQRPS

SEQ ID NO: 221 - protein, 7 aa, synthetic construct
EDNQRPS

SEQ ID NO: 222 - protein, 7 aa, synthetic construct
EDNQRPS

SEQ ID NO: 223 - protein, 7 aa, synthetic construct
EDNQRPS

SEQ ID NO: 224 - protein, 7 aa, synthetic construct
GASSRAT

SEQ ID NO: 225 - protein, 7 aa, synthetic construct
STNTHSS

SEQ ID NO: 226 - protein, 7 aa, synthetic construct
STNTHSS

SEQ ID NO: 227 - protein, 9 aa, synthetic construct
QSYDSSNRV

SEQ ID NO: 228 - protein, 9 aa, synthetic construct
QAWDISTVV

SEQ ID NO: 229 - protein, 9 aa, synthetic construct
QAWDSSTVV

SEQ ID NO: 230 - protein, 9 aa, synthetic construct
QAWDSSTVV

SEQ ID NO: 231 - protein, 9 aa, synthetic construct
QSYDSSNHV

SEQ ID NO: 232 - protein, 12 aa, synthetic construct
YSTDSSGNHSWV

SEQ ID NO: 233 - protein, 9 aa, synthetic construct
QSFDNSNRV

SEQ ID NO: 234 - protein, 12 aa, synthetic construct
YSTDSSGNHSWV

SEQ ID NO: 235 - protein, 12 aa, synthetic construct
YSTDSSGNHCWV

SEQ ID NO: 236 - protein, 9 aa, synthetic construct
QSLDSSSVV

SEQ ID NO: 237 - protein, 9 aa, synthetic construct
QSYDSSNQV

SEQ ID NO: 238 - protein, 9 aa, synthetic construct
QSFDNSNRV

SEQ ID NO: 239 - protein, 9 aa, synthetic construct
QQYKSYSWT

SEQ ID NO: 240 - protein, 9 aa, synthetic construct
QSYDSSNRV

SEQ ID NO: 241 - protein, 9 aa, synthetic construct
QSYDSSNRV

SEQ ID NO: 242 - protein, 9 aa, synthetic construct
QQYNSYSWT

SEQ ID NO: 243 - protein, 9 aa, synthetic construct
QSYDSNNRV

SEQ ID NO: 244 - protein, 9 aa, synthetic construct
MQSTQFPIT

SEQ ID NO: 245 - protein, 9 aa, synthetic construct
QSFDNSNRV

SEQ ID NO: 246 - protein, 9 aa, synthetic construct
QSYDRSNRV

SEQ ID NO: 247 - protein, 11 aa, synthetic construct
YSTHSSGNHRV

SEQ ID NO: 248 - protein, 11 aa, synthetic construct
QSADSSGTYRV

SEQ ID NO: 249 - protein, 9 aa, synthetic construct
QSFDSSNRV

SEQ ID NO: 250 - protein, 9 aa, synthetic construct
QSFYSSNRV

SEQ ID NO: 251 - protein, 9 aa, synthetic construct
QSFDSSNRV

SEQ ID NO: 252 - protein, 9 aa, synthetic construct
QSFDSSNRV

SEQ ID NO: 253 - protein, 10 aa, synthetic construct
VLYMGSGIWV

SEQ ID NO: 254 - protein, 9 aa, synthetic construct
QSFDSSNVV

SEQ ID NO: 255 - protein, 11 aa, synthetic construct
EAWDDSLSGPV

SEQ ID NO: 256 - protein, 9 aa, synthetic construct
QSYNSSNQV

SEQ ID NO: 257 - protein, 9 aa, synthetic construct
MQPLQTPYT

SEQ ID NO: 258 - protein, 9 aa, synthetic construct
QAWDSSTVV

SEQ ID NO: 259 - protein, 9 aa, synthetic construct
QSYYNSNRV

SEQ ID NO: 260 - protein, 9 aa, synthetic construct
QSYDSSNRV

SEQ ID NO: 261 - protein, 12 aa, synthetic construct
YSTDSSGNHCWV

SEQ ID NO: 262 - protein, 9 aa, synthetic construct
QQYISYSWT

SEQ ID NO: 263 - protein, 8 aa, synthetic construct
QQYNSYWT

SEQ ID NO: 264 - protein, 11 aa, synthetic construct
QSYDSSLSGYV

SEQ ID NO: 265 - protein, 9 aa, synthetic construct
MQGTHWPLT

SEQ ID NO: 266 - protein, 11 aa, synthetic construct
APWDDSLNGVV

SEQ ID NO: 267 - protein, 12 aa, synthetic construct
YSTDSSGNHCWV

SEQ ID NO: 268 - protein, 11 aa, synthetic construct
QSADSSGTYRV

SEQ ID NO: 269 - protein, 11 aa, synthetic construct
CSYAGSTTFAI

SEQ ID NO: 270 - protein, 11 aa, synthetic construct
GTWHSNSKTWV

SEQ ID NO: 271 - protein, 9 aa, synthetic construct
QQYKSYSWT

SEQ ID NO: 272 - protein, 7 aa, synthetic construct
YDRSNRV

SEQ ID NO: 273 - protein, 9 aa, synthetic construct
QSYYSNNRV

SEQ ID NO: 274 - protein, 9 aa, synthetic construct
QSFDSSNRV

SEQ ID NO: 275 - protein, 9 aa, synthetic construct
QCYDRSNRV

SEQ ID NO: 276 - protein, 9 aa, synthetic construct
QSFDTSNVV

SEQ ID NO: 277 - protein, 9 aa, synthetic construct
QSYDSSNRV

SEQ ID NO: 278 - protein, 11 aa, synthetic construct
YSTDSSGNHRV

SEQ ID NO: 279 - protein, 9 aa, synthetic construct
QSYDSSNRV

SEQ ID NO: 280 - protein, 9 aa, synthetic construct
QSFDSSNRV

SEQ ID NO: 281 - protein, 9 aa, synthetic construct
QSYDSSNRV

SEQ ID NO: 282 - protein, 9 aa, synthetic construct
QSYDSSNRV

SEQ ID NO: 283 - protein, 11 aa, synthetic construct
GTWHSNSKTQV

SEQ ID NO: 284 - protein, 9 aa, synthetic construct
QSFDSSNRV

SEQ ID NO: 285 - protein, 9 aa, synthetic construct
QSYDNSIRV

SEQ ID NO: 286 - protein, 9 aa, synthetic construct
QSYDSSNRV

SEQ ID NO: 287 - protein, 9 aa, synthetic construct
LLYYGGAYV

SEQ ID NO: 288 - protein, 9 aa, synthetic construct
QSYDSSNRV

SEQ ID NO: 289 - protein, 9 aa, synthetic construct
QSYDRSNRV

SEQ ID NO: 290 - protein, 9 aa, synthetic construct
QSYDSSNRV

SEQ ID NO: 291 - protein, 11 aa, synthetic construct
AAWDDSLSGVV

SEQ ID NO: 292 - protein, 9 aa, synthetic construct
QSYDRSNRV

SEQ ID NO: 293 - protein, 9 aa, synthetic construct
QSYDSSNRV

SEQ ID NO: 294 - protein, 11 aa, synthetic construct
QSADSSGTYVV

SEQ ID NO: 295 - protein, 9 aa, synthetic construct
QSYDSSNRV

SEQ ID NO: 296 - protein, 9 aa, synthetic construct
QSYDSSNRV

SEQ ID NO: 297 - protein, 9 aa, synthetic construct
QSYDRSNRV

SEQ ID NO: 298 - protein, 9 aa, synthetic construct
QSYDSSNRV

SEQ ID NO: 299 - protein, 9 aa, synthetic construct
QSYDNSNRV

SEQ ID NO: 300 - protein, 11 aa, synthetic construct
YSTDSSGNHWV

SEQ ID NO: 301 - protein, 12 aa, synthetic construct
YSTDSSGNHNWV

SEQ ID NO: 302 - protein, 11 aa, synthetic construct
YSTDSSGNHWV

SEQ ID NO: 303 - protein, 11 aa, synthetic construct
QSYDSSLSGSV

SEQ ID NO: 304 - protein, 9 aa, synthetic construct
QSFDGSNRV

SEQ ID NO: 305 - protein, 10 aa, synthetic construct
VLYMGSGIWV

SEQ ID NO: 306 - protein, 12 aa, synthetic construct
YSTDSSGNHSWV

SEQ ID NO: 307 - protein, 9 aa, synthetic construct
QSFDSSNRV

SEQ ID NO: 308 - protein, 12 aa, synthetic construct
YSTDSSGNHSWV

SEQ ID NO: 309 - protein, 9 aa, synthetic construct
QSSDRSNVV

SEQ ID NO: 310 - protein, 9 aa, synthetic construct
QSYDSSNRV

SEQ ID NO: 311 - protein, 9 aa, synthetic construct
QSCDSSNVV

SEQ ID NO: 312 - protein, 9 aa, synthetic construct
QSCDSSNVV

SEQ ID NO: 313 - protein, 9 aa, synthetic construct
QSYDSSNRV

SEQ ID NO: 314 - protein, 9 aa, synthetic construct
QQYNSYSWT

SEQ ID NO: 315 - protein, 9 aa, synthetic construct
QQYGSSRWS

SEQ ID NO: 316 - protein, 9 aa, synthetic construct
QSYDSSNQV

SEQ ID NO: 317 - protein, 9 aa, synthetic construct
QSYDSSNQV

SEQ ID NO: 318 - protein, 9 aa, synthetic construct
QAWDSTTAV

SEQ ID NO: 319 - protein, 9 aa, synthetic construct
QSYDSSNQV

SEQ ID NO: 320 - protein, 9 aa, synthetic construct
QSYDSSIRV

SEQ ID NO: 321 - protein, 9 aa, synthetic construct
QSYDSSNQV

SEQ ID NO: 322 - protein, 9 aa, synthetic construct
QSYDSSNVV

SEQ ID NO: 323 - protein, 9 aa, synthetic construct
QAWDSTTAV

SEQ ID NO: 324 - protein, 9 aa, synthetic construct
QSYDNNNRV

SEQ ID NO: 325 - protein, 9 aa, synthetic construct
QSYDSSIRV

SEQ ID NO: 326 - protein, 9 aa, synthetic construct
QAWDSITAV

SEQ ID NO: 327 - protein, 9 aa, synthetic construct
QSYDSSNQV

SEQ ID NO: 328 - protein, 9 aa, synthetic construct
QSYDRSIRV

SEQ ID NO: 329 - protein, 9 aa, synthetic construct
QSYDSSNVV

SEQ ID NO: 330 - protein, 9 aa, synthetic construct
QSYDSSNVV

SEQ ID NO: 331 - protein, 9 aa, synthetic construct
QSYDSSNQV

SEQ ID NO: 332 - protein, 9 aa, synthetic construct
QSYDGSIRV

SEQ ID NO: 333 - protein, 12 aa, synthetic construct
AAWDDSLNGRVV

SEQ ID NO: 334 - protein, 9 aa, synthetic construct
QSYDSSNVV

SEQ ID NO: 335 - protein, 9 aa, synthetic construct
QSYDSSNQV

SEQ ID NO: 336 - protein, 9 aa, synthetic construct
QSYDGSNRV

SEQ ID NO: 337 - protein, 9 aa, synthetic construct
QQYGSSPFT

SEQ ID NO: 338 - protein, 10 aa, synthetic construct
VLYMGSGIWV

SEQ ID NO: 339 - protein, 10 aa, synthetic construct
VLYMGSGIWV

SEQ ID NO: 340 - protein, 12 aa, synthetic construct
YSTDSSGNHSWV

SEQ ID NO: 341 - protein, 11 aa, synthetic construct
SSYAGSTTFAI

SEQ ID NO: 342 - protein, 9 aa, synthetic construct
QSYDRSNRV

SEQ ID NO: 343 - protein, 9 aa, synthetic construct
QSYDSSNVV

SEQ ID NO: 344 - protein, 9 aa, synthetic construct
QSSDSSNVV

SEQ ID NO: 345 - protein, 5 aa, synthetic construct
SYSMN

SEQ ID NO: 346 - protein, 5 aa, synthetic construct
SYSMN

SEQ ID NO: 347 - protein, 5 aa, synthetic construct
SYSMN

SEQ ID NO: 348 - protein, 5 aa, synthetic construct
SYNMN

SEQ ID NO: 349 - protein, 5 aa, synthetic construct
RYSMN

SEQ ID NO: 350 - protein, 5 aa, synthetic construct
SYSMN

SEQ ID NO: 351 - protein, 5 aa, synthetic construct
RYSMN

SEQ ID NO: 352 - protein, 5 aa, synthetic construct
SYSMN

SEQ ID NO: 353 - protein, 5 aa, synthetic construct
SYSMN

SEQ ID NO: 354 - protein, 5 aa, synthetic construct
CNSVA

SEQ ID NO: 355 - protein, 7 aa, synthetic construct
TFGMGVG

SEQ ID NO: 356 - protein, 5 aa, synthetic construct
SYTMN

SEQ ID NO: 357 - protein, 5 aa, synthetic construct
NYWMS

SEQ ID NO: 358 - protein, 5 aa, synthetic construct
SYSMT

SEQ ID NO: 359 - protein, 5 aa, synthetic construct
SYSMN

SEQ ID NO: 360 - protein, 5 aa, synthetic construct
RYWMT

SEQ ID NO: 361 - protein, 5 aa, synthetic construct
RYSIN

SEQ ID NO: 362 - protein, 5 aa, synthetic construct
SYAMH

SEQ ID NO: 363 - protein, 5 aa, synthetic construct
SYSMN

SEQ ID NO: 364 - protein, 5 aa, synthetic construct
TYSMN

SEQ ID NO: 365 - protein, 5 aa, synthetic construct
SYSMN

SEQ ID NO: 366 - protein, 5 aa, synthetic construct
SYSMN

SEQ ID NO: 367 - protein, 5 aa, synthetic construct
SYSMN

SEQ ID NO: 368 - protein, 5 aa, synthetic construct
SYTMN

SEQ ID NO: 369 - protein, 5 aa, synthetic construct
SYSMN

SEQ ID NO: 370 - protein, 5 aa, synthetic construct
SYSMN

SEQ ID NO: 371 - protein, 5 aa, synthetic construct
SYYMH

SEQ ID NO: 372 - protein, 5 aa, synthetic construct
SNSVA

SEQ ID NO: 373 - protein, 5 aa, synthetic construct
SYWIA

SEQ ID NO: 374 - protein, 5 aa, synthetic construct
SYYWS

SEQ ID NO: 375 - protein, 5 aa, synthetic construct
NYWIG

SEQ ID NO: 376 - protein, 5 aa, synthetic construct
SYNMN

SEQ ID NO: 377 - protein, 5 aa, synthetic construct
SYSMN

SEQ ID NO: 378 - protein, 5 aa, synthetic construct
RYSMN

SEQ ID NO: 379 - protein, 5 aa, synthetic construct
SYSMN

SEQ ID NO: 380 - protein, 5 aa, synthetic construct
NYWMS

SEQ ID NO: 381 - protein, 5 aa, synthetic construct
SYSMN

SEQ ID NO: 382 - protein, 5 aa, synthetic construct
GYYWS

SEQ ID NO: 383 - protein, 5 aa, synthetic construct
YYAMS

SEQ ID NO: 384 - protein, 5 aa, synthetic construct
YYAMS

SEQ ID NO: 385 - protein, 5 aa, synthetic construct
SYSMN

SEQ ID NO: 386 - protein, 5 aa, synthetic construct
SYSIN

SEQ ID NO: 387 - protein, 5 aa, synthetic construct
TYWIA

SEQ ID NO: 388 - protein, 5 aa, synthetic construct
TYWMS

SEQ ID NO: 389 - protein, 5 aa, synthetic construct
SYWMS

SEQ ID NO: 390 - protein, 5 aa, synthetic construct
TYTMN

SEQ ID NO: 391 - protein, 5 aa, synthetic construct
RYSMN

SEQ ID NO: 392 - protein, 5 aa, synthetic construct
SYTMN

SEQ ID NO: 393 - protein, 5 aa, synthetic construct
TYTMN

SEQ ID NO: 394 - protein, 5 aa, synthetic construct
SNSVA

SEQ ID NO: 395 - protein, 5 aa, synthetic construct
SYSMN

SEQ ID NO: 396 - protein, 5 aa, synthetic construct
YYSMN

SEQ ID NO: 397 - protein, 5 aa, synthetic construct
SYTMN

SEQ ID NO: 398 - protein, 5 aa, synthetic construct
SYSMN

SEQ ID NO: 399 - protein, 5 aa, synthetic construct
IYSMN

SEQ ID NO: 400 - protein, 5 aa, synthetic construct
RYSMN

SEQ ID NO: 401 - protein, 5 aa, synthetic construct
SYTMN

SEQ ID NO: 402 - protein, 5 aa, synthetic construct
NYNMN

SEQ ID NO: 403 - protein, 5 aa, synthetic construct
SYSMN

SEQ ID NO: 404 - protein, 5 aa, synthetic construct
IYSMN

SEQ ID NO: 405 - protein, 5 aa, synthetic construct
SYSMN

SEQ ID NO: 406 - protein, 5 aa, synthetic construct
SYSMN

SEQ ID NO: 407 - protein, 5 aa, synthetic construct
SYYWS

SEQ ID NO: 408 - protein, 5 aa, synthetic construct
RYSMN

SEQ ID NO: 409 - protein, 5 aa, synthetic construct
GYYWS

SEQ ID NO: 410 - protein, 5 aa, synthetic construct
SYYWS

SEQ ID NO: 411 - protein, 5 aa, synthetic construct
SYSMN

SEQ ID NO: 412 - protein, 5 aa, synthetic construct
SSIMN

SEQ ID NO: 413 - protein, 5 aa, synthetic construct
TYTMN

SEQ ID NO: 414 - protein, 5 aa, synthetic construct
TYIMN

SEQ ID NO: 415 - protein, 5 aa, synthetic construct
SYYWS

SEQ ID NO: 416 - protein, 5 aa, synthetic construct
TYIMN

SEQ ID NO: 417 - protein, 5 aa, synthetic construct
SYSMN

SEQ ID NO: 418 - protein, 5 aa, synthetic construct
SYSMK

SEQ ID NO: 419 - protein, 5 aa, synthetic construct
TYSLN

SEQ ID NO: 420 - protein, 5 aa, synthetic construct
NYYMN

SEQ ID NO: 421 - protein, 5 aa, synthetic construct
SYSMN

SEQ ID NO: 422 - protein, 5 aa, synthetic construct
NYSMN

SEQ ID NO: 423 - protein, 5 aa, synthetic construct
NYSMN

SEQ ID NO: 424 - protein, 5 aa, synthetic construct
GYSMN

SEQ ID NO: 425 - protein, 5 aa, synthetic construct
SGGYY

SEQ ID NO: 426 - protein, 5 aa, synthetic construct
SYSMN

SEQ ID NO: 427 - protein, 5 aa, synthetic construct
SYSMN

SEQ ID NO: 428 - protein, 5 aa, synthetic construct
NFIMN

SEQ ID NO: 429 - protein, 5 aa, synthetic construct
SYSMN

SEQ ID NO: 430 - protein, 5 aa, synthetic construct
RYSMN

SEQ ID NO: 431 - protein, 5 aa, synthetic construct
RYSMN

SEQ ID NO: 432 - protein, 5 aa, synthetic construct
RYWMS

SEQ ID NO: 433 - protein, 5 aa, synthetic construct
SYWMS

SEQ ID NO: 434 - protein, 5 aa, synthetic construct
SYSMN

SEQ ID NO: 435 - protein, 5 aa, synthetic construct
YYSMN

SEQ ID NO: 436 - protein, 5 aa, synthetic construct
SYSMN

SEQ ID NO: 437 - protein, 5 aa, synthetic construct
RYSMN

SEQ ID NO: 438 - protein, 5 aa, synthetic construct
SYSMN

SEQ ID NO: 439 - protein, 5 aa, synthetic construct
RYSMN

SEQ ID NO: 440 - protein, 5 aa, synthetic construct
RYSMN

SEQ ID NO: 441 - protein, 5 aa, synthetic construct
SYSMN

SEQ ID NO: 442 - protein, 5 aa, synthetic construct
SYSMN

SEQ ID NO: 443 - protein, 5 aa, synthetic construct
SYSMN

SEQ ID NO: 444 - protein, 5 aa, synthetic construct
SYSMN

SEQ ID NO: 445 - protein, 5 aa, synthetic construct
RYSMN

SEQ ID NO: 446 - protein, 5 aa, synthetic construct
SYSMN

SEQ ID NO: 447 - protein, 5 aa, synthetic construct
RYSMN

SEQ ID NO: 448 - protein, 5 aa, synthetic construct
RYSMN

SEQ ID NO: 449 - protein, 5 aa, synthetic construct
RYSMN

SEQ ID NO: 450 - protein, 5 aa, synthetic construct
SYSMN

SEQ ID NO: 451 - protein, 5 aa, synthetic construct
TYWIA

SEQ ID NO: 452 - protein, 5 aa, synthetic construct
RYSMN

SEQ ID NO: 453 - protein, 5 aa, synthetic construct
SYSMN

SEQ ID NO: 454 - protein, 5 aa, synthetic construct
SYSMN

SEQ ID NO: 455 - protein, 5 aa, synthetic construct
SYSMN

SEQ ID NO: 456 - protein, 5 aa, synthetic construct
SYSMN

SEQ ID NO: 457 - protein, 5 aa, synthetic construct
SYSMN

SEQ ID NO: 458 - protein, 17 aa, synthetic construct
SISRSGSYIYYADSVKG

SEQ ID NO: 459 - protein, 17 aa, synthetic construct
SISRSTSYIYYADSVKG

SEQ ID NO: 460 - protein, 17 aa, synthetic construct
YISRSSNYIHYADSVKG

SEQ ID NO: 461 - protein, 17 aa, synthetic construct
SISSSSSYIYYADSVKG

SEQ ID NO: 462 - protein, 17 aa, synthetic construct
YISSSSSSIYYADSVKG

SEQ ID NO: 463 - protein, 17 aa, synthetic construct
SISSRGSYRNYADSVKG

SEQ ID NO: 464 - protein, 17 aa, synthetic construct
SISSSGSYKYYADSVKG

SEQ ID NO: 465 - protein, 17 aa, synthetic construct
SISSSSSYIYYADSVKG

SEQ ID NO: 466 - protein, 17 aa, synthetic construct
SVNSRGSYIYYADSVKG

SEQ ID NO: 467 - protein, 19 aa, synthetic construct
LGRTYYRSKWHNEYAVSVK

SEQ ID NO: 468 - protein, 16 aa, synthetic construct
HIWWDDDKYYNPALKS

SEQ ID NO: 469 - protein, 17 aa, synthetic construct
SISRSSSYKYYADSVKG

SEQ ID NO: 470 - protein, 17 aa, synthetic construct
NIKEDGSEKYYVDSVKG

SEQ ID NO: 471 - protein, 17 aa, synthetic construct
SISRRSSYIYYADSVKG

SEQ ID NO: 472 - protein, 17 aa, synthetic construct
SISRSSNYIYYADSVKG

SEQ ID NO: 473 - protein, 17 aa, synthetic construct
NIKEDGSEKYCVDSVKG

SEQ ID NO: 474 - protein, 17 aa, synthetic construct
YISSSSSTIYYADSVKG

SEQ ID NO: 475 - protein, 17 aa, synthetic construct
AISSNGGRTYYADSVKG

SEQ ID NO: 476 - protein, 17 aa, synthetic construct
SISRSSSYIYYADSVKG

SEQ ID NO: 477 - protein, 17 aa, synthetic construct
SISSSGSYVYYADSVKG

SEQ ID NO: 478 - protein, 17 aa, synthetic construct
SISRSSSYIYYADSVKG

SEQ ID NO: 479 - protein, 17 aa, synthetic construct
YISSSSSTIYYADSVKG

SEQ ID NO: 480 - protein, 17 aa, synthetic construct
SISSSSTYIYYADSVKG

SEQ ID NO: 481 - protein, 17 aa, synthetic construct
SISRSGSYKYNADSVKG

SEQ ID NO: 482 - protein, 17 aa, synthetic construct
SISSSSTYIYYADSVKG

SEQ ID NO: 483 - protein, 17 aa, synthetic construct
SISSSSTYIYYADSVKG

SEQ ID NO: 484 - protein, 17 aa, synthetic construct
IINPSNGNTISAQKFQG

SEQ ID NO: 485 - protein, 19 aa, synthetic construct
LGRTYYRSKWYNECAGSVK

SEQ ID NO: 486 - protein, 19 aa, synthetic construct
IIYPGDSETRYRPSFQGQV

SEQ ID NO: 487 - protein, 16 aa, synthetic construct
HIYYSGRTKYNPSLKS

SEQ ID NO: 488 - protein, 19 aa, synthetic construct
IIYPADSDTTYSPSFQGQV

SEQ ID NO: 489 - protein, 17 aa, synthetic construct
SISSSSSYIYYTDSVKG

SEQ ID NO: 490 - protein, 17 aa, synthetic construct
SISRSSSYIYYADSVKG

SEQ ID NO: 491 - protein, 17 aa, synthetic construct
SISRSSSYIYYADSVKG

SEQ ID NO: 492 - protein, 17 aa, synthetic construct
SVNSRGSYIYYADSVKG

SEQ ID NO: 493 - protein, 17 aa, synthetic construct
NIKEDGSEKYCVDSVKG

SEQ ID NO: 494 - protein, 17 aa, synthetic construct
HIRSSTYSIYYADSVMG

SEQ ID NO: 495 - protein, 16 aa, synthetic construct
EINHSGNTNYNPSLKS

SEQ ID NO: 496 - protein, 17 aa, synthetic construct
AISGSGGSTYYADSVKG

SEQ ID NO: 497 - protein, 17 aa, synthetic construct
AISGSGGSTYYADSVKG

SEQ ID NO: 498 - protein, 17 aa, synthetic construct
SVNSRGSYIYYADSVKG

SEQ ID NO: 499 - protein, 17 aa, synthetic construct
YISSSSSTIYYADSVKG

SEQ ID NO: 500 - protein, 19 aa, synthetic construct
IIYPGDSDTTYSPSFQGQV

SEQ ID NO: 501 - protein, 17 aa, synthetic construct
NIKQDGSEKYYVDSVKG

SEQ ID NO: 502 - protein, 17 aa, synthetic construct
NIKEDGSEKYYVDSVKG

SEQ ID NO: 503 - protein, 17 aa, synthetic construct
SISKSSTYIYYADSVKG

SEQ ID NO: 504 - protein, 17 aa, synthetic construct
YISRSSTTIYYADSVKG

SEQ ID NO: 505 - protein, 17 aa, synthetic construct
SISGSGSYIYYADSVKG

SEQ ID NO: 506 - protein, 17 aa, synthetic construct
SISKSSSYIYYTDSVKG

SEQ ID NO: 507 - protein, 19 aa, synthetic construct
LGRTYSRSKWYNDYAVSVK

SEQ ID NO: 508 - protein, 17 aa, synthetic construct
SISRSSSYIYYADSVKG

SEQ ID NO: 509 - protein, 17 aa, synthetic construct
SISRSTTYIYYADSVKG

SEQ ID NO: 510 - protein, 17 aa, synthetic construct
SITTSSSYIYYADSVKG

SEQ ID NO: 511 - protein, 17 aa, synthetic construct
SISRSSRYIFYADSVRG

SEQ ID NO: 512 - protein, 17 aa, synthetic construct
SISSGSSYIYYADSVKG

SEQ ID NO: 513 - protein, 17 aa, synthetic construct
SISSSSRYTYYADSLKG

SEQ ID NO: 514 - protein, 17 aa, synthetic construct
SISTSSRYIYYTDSVKG

SEQ ID NO: 515 - protein, 16 aa, synthetic construct
SSSRSSYIYYADSVKG

SEQ ID NO: 516 - protein, 17 aa, synthetic construct
SLSRSSNYIYYAVSVKG

SEQ ID NO: 517 - protein, 17 aa, synthetic construct
SISSGSSYIYYADSVKG

SEQ ID NO: 518 - protein, 17 aa, synthetic construct
SISRSGTYIYYADSVKG

SEQ ID NO: 519 - protein, 17 aa, synthetic construct
SISRSSGYIFYADSVKG

SEQ ID NO: 520 - protein, 16 aa, synthetic construct
RIYTSGSTKYNPSLKS

SEQ ID NO: 521 - protein, 17 aa, synthetic construct
SISSSGSYTYYADSVKG

SEQ ID NO: 522 - protein, 16 aa, synthetic construct
EINHSGSTNYNPSLKS

SEQ ID NO: 523 - protein, 16 aa, synthetic construct
RIYASGSTYYNPSLKS

SEQ ID NO: 524 - protein, 17 aa, synthetic construct
SISRSSSYIYYADSVKG

SEQ ID NO: 525 - protein, 17 aa, synthetic construct
SISGSSSYIYYADSVKG

SEQ ID NO: 526 - protein, 17 aa, synthetic construct
TITSSSRYIYYADSVKG

SEQ ID NO: 527 - protein, 17 aa, synthetic construct
SITSSSSYIYYADSVKG

SEQ ID NO: 528 - protein, 16 aa, synthetic construct
RIYTSGSTNYNPSLKS

SEQ ID NO: 529 - protein, 17 aa, synthetic construct
SISSSRSYIYYADSVKG

SEQ ID NO: 530 - protein, 17 aa, synthetic construct
TFSRSSTYIYYADSVKG

SEQ ID NO: 531 - protein, 17 aa, synthetic construct
SISSSSSYIYYADSVKG

SEQ ID NO: 532 - protein, 17 aa, synthetic construct
SISVSSRYIYFADSVKG

SEQ ID NO: 533 - protein, 17 aa, synthetic construct
SISSSSNYIYYADSVKG

SEQ ID NO: 534 - protein, 17 aa, synthetic construct
SISSSSSYIYYADSVKG

SEQ ID NO: 535 - protein, 17 aa, synthetic construct
CISRSSSYIYYADSVKG

SEQ ID NO: 536 - protein, 17 aa, synthetic construct
CISRSSSYIYYADSVKG

SEQ ID NO: 537 - protein, 17 aa, synthetic construct
SISSRSRYIYYADSVQG

SEQ ID NO: 538 - protein, 17 aa, synthetic construct
IGYIYYSGSTYYNPSLK

SEQ ID NO: 539 - protein, 17 aa, synthetic construct
SISSSSSYIYYADSVKG

SEQ ID NO: 540 - protein, 17 aa, synthetic construct
TISRSGGYIYDADSVKG

SEQ ID NO: 541 - protein, 17 aa, synthetic construct
SISGSSNYIYYADSVKG

SEQ ID NO: 542 - protein, 17 aa, synthetic construct
SISRRSGYIYYADSVKG

SEQ ID NO: 543 - protein, 17 aa, synthetic construct
TISRSSRYIYYGDSVKG

SEQ ID NO: 544 - protein, 17 aa, synthetic construct
SISSSSSYKYYADSVKG

SEQ ID NO: 545 - protein, 17 aa, synthetic construct
NIKEDGSEKYYVDSVKG

SEQ ID NO: 546 - protein, 17 aa, synthetic construct
SISSRRGYKYHADSVKG

SEQ ID NO: 547 - protein, 17 aa, synthetic construct
SISSSSSYIYYADSVKG

SEQ ID NO: 548 - protein, 17 aa, synthetic construct
SISSSSTYIYYADSVKG

SEQ ID NO: 549 - protein, 17 aa, synthetic construct
YISSSSRYIYYADSVKG

SEQ ID NO: 550 - protein, 17 aa, synthetic construct
SISSSSSYIYYADSVKG

SEQ ID NO: 551 - protein, 17 aa, synthetic construct
SISRSSSYIFYADSVKG

SEQ ID NO: 552 - protein, 17 aa, synthetic construct
SISRSSSYIYYADSVKG

SEQ ID NO: 553 - protein, 17 aa, synthetic construct
SISSSSSYIYYADSVKG

SEQ ID NO: 554 - protein, 17 aa, synthetic construct
YISRSSTYIYYADSMKG

SEQ ID NO: 555 - protein, 17 aa, synthetic construct
SISSSSSYIYYADSVKG

SEQ ID NO: 556 - protein, 17 aa, synthetic construct
SISSSSSYIYYADSVKG

SEQ ID NO: 557 - protein, 17 aa, synthetic construct
YISSSSNYIYYADSVKG

SEQ ID NO: 558 - protein, 17 aa, synthetic construct
SISSSSSYIYYADSVKG

SEQ ID NO: 559 - protein, 17 aa, synthetic construct
SISSSSSYIYYADSVKG

SEQ ID NO: 560 - protein, 17 aa, synthetic construct
SISSSSSYISYADSVKG

SEQ ID NO: 561 - protein, 17 aa, synthetic construct
SISSSSSYIYYADSVKG

SEQ ID NO: 562 - protein, 17 aa, synthetic construct
SISSSSSYIYYADSVKG

SEQ ID NO: 563 - protein, 17 aa, synthetic construct
SISRSSSYIFYADSVKG

SEQ ID NO: 564 - protein, 19 aa, synthetic construct
FIYPGDSDTIYSPSFQGQV

SEQ ID NO: 565 - protein, 17 aa, synthetic construct
SISSSSRYIYYADSVKG

SEQ ID NO: 566 - protein, 17 aa, synthetic construct
SISSSSSYIYYADSVKG

SEQ ID NO: 567 - protein, 17 aa, synthetic construct
SISSSSSYIYYADSVKG

SEQ ID NO: 568 - protein, 17 aa, synthetic construct
YISRSSTYIYYADSMKG

SEQ ID NO: 569 - protein, 17 aa, synthetic construct
SISSSSSYIYYADSVKG

SEQ ID NO: 570 - protein, 17 aa, synthetic construct
SISSSSSYIYYADSVKG

SEQ ID NO: 571 - protein, 17 aa, synthetic construct
NIKEDGSEKYYVDSVKG

SEQ ID NO: 572 - protein, 16 aa, synthetic construct
EIQHSGSTNYNPSLKS

SEQ ID NO: 573 - protein, 17 aa, synthetic construct
SISRSSSYIYYADSVKG

SEQ ID NO: 574 - protein, 10 aa, synthetic construct
EKWELLYFDY

SEQ ID NO: 575 - protein, 8 aa, synthetic construct
GSRDAFDI

SEQ ID NO: 576 - protein, 10 aa, synthetic construct
AERGSYAVDI

SEQ ID NO: 577 - protein, 10 aa, synthetic construct
AERGSYAFDI

SEQ ID NO: 578 - protein, 8 aa, synthetic construct
DRRATLDY

SEQ ID NO: 579 - protein, 9 aa, synthetic construct
DRRELLLDC

SEQ ID NO: 580 - protein, 9 aa, synthetic construct
EPPTLGMDY

SEQ ID NO: 581 - protein, 9 aa, synthetic construct
DRRELLLDY

SEQ ID NO: 582 - protein, 9 aa, synthetic construct
DRRELLLDY

SEQ ID NO: 583 - protein, 10 aa, synthetic construct
ERKWELFLDY

SEQ ID NO: 584 - protein, 11 aa, synthetic construct
LGYYPYWFFDV

SEQ ID NO: 585 - protein, 9 aa, synthetic construct
EPPTLGMDY

SEQ ID NO: 586 - protein, 9 aa, synthetic construct
ERRTGYFDY

SEQ ID NO: 587 - protein, 10 aa, synthetic construct
ERWELLYFDY

SEQ ID NO: 588 - protein, 10 aa, synthetic construct
ERWIQLYSDY

SEQ ID NO: 589 - protein, 9 aa, synthetic construct
ERRTGYFDY

SEQ ID NO: 590 - protein, 10 aa, synthetic construct
ERWELLYFDY

SEQ ID NO: 591 - protein, 6 aa, synthetic construct
GGSLDY

SEQ ID NO: 592 - protein, 10 aa, synthetic construct
ERWELLYFDY

SEQ ID NO: 593 - protein, 9 aa, synthetic construct
ERRELLFDY

SEQ ID NO: 594 - protein, 10 aa, synthetic construct
DGKGAIFLDY

SEQ ID NO: 595 - protein, 10 aa, synthetic construct
DGPVDYGMDV

SEQ ID NO: 596 - protein, 9 aa, synthetic construct
EPRTLGMDY

SEQ ID NO: 597 - protein, 9 aa, synthetic construct
EPPTLGMDY

SEQ ID NO: 598 - protein, 9 aa, synthetic construct
EPRTLGMDY

SEQ ID NO: 599 - protein, 9 aa, synthetic construct
EPRTLGMDY

SEQ ID NO: 600 - protein, 9 aa, synthetic construct
CWGYYVMDV

SEQ ID NO: 601 - protein, 10 aa, synthetic construct
ERKWELFLDY

SEQ ID NO: 602 - protein, 9 aa, synthetic construct
QGAYDGFDI

SEQ ID NO: 603 - protein, 9 aa, synthetic construct
ENRNGAFDI

SEQ ID NO: 604 - protein, 12 aa, synthetic construct
HGGSYILDAFDI

SEQ ID NO: 605 - protein, 10 aa, synthetic construct
AERGSYAFDF

SEQ ID NO: 606 - protein, 9 aa, synthetic construct
EPPTLGMDY

SEQ ID NO: 607 - protein, 9 aa, synthetic construct
EPRTLGMDY

SEQ ID NO: 608 - protein, 9 aa, synthetic construct
DRRELLLDY

SEQ ID NO: 609 - protein, 9 aa, synthetic construct
ERRTGYFDY

SEQ ID NO: 610 - protein, 10 aa, synthetic construct
AKTGGDAFDI

SEQ ID NO: 611 - protein, 10 aa, synthetic construct
EVEIWGYFDY

SEQ ID NO: 612 - protein, 13 aa, synthetic construct
EGIVGARAEYFQH

SEQ ID NO: 613 - protein, 13 aa, synthetic construct
EGIVGARAEYFQH

SEQ ID NO: 614 - protein, 9 aa, synthetic construct
DRRELLLDY

SEQ ID NO: 615 - protein, 10 aa, synthetic construct
DGPVDYGMDV

SEQ ID NO: 616 - protein, 9 aa, synthetic construct
QGELDAFDI

SEQ ID NO: 617 - protein, 5 aa, synthetic construct
GSFDY

SEQ ID NO: 618 - protein, 9 aa, synthetic construct
ERRTGYLDY

SEQ ID NO: 619 - protein, 9 aa, synthetic construct
ERRTLAFDI

SEQ ID NO: 620 - protein, 10 aa, synthetic construct
ERWELLYFDY

SEQ ID NO: 621 - protein, 9 aa, synthetic construct
EPPTLGMDY

SEQ ID NO: 622 - protein, 9 aa, synthetic construct
ERRTLAFDI

SEQ ID NO: 623 - protein, 10 aa, synthetic construct
ERKWELFLDY

SEQ ID NO: 624 - protein, 10 aa, synthetic construct
ERWELLYFDY

SEQ ID NO: 625 - protein, 10 aa, synthetic construct
DGKGAIFLDY

SEQ ID NO: 626 - protein, 8 aa, synthetic construct
VTFDAFDI

SEQ ID NO: 627 - protein, 9 aa, synthetic construct
EKRELLFDY

SEQ ID NO: 628 - protein, 9 aa, synthetic construct
EKRELLFDY

SEQ ID NO: 629 - protein, 8 aa, synthetic construct
NLYNALDY

SEQ ID NO: 630 - protein, 9 aa, synthetic construct
NWGYDVFDI

SEQ ID NO: 631 - protein, 10 aa, synthetic construct
EKWELLFFDY

SEQ ID NO: 632 - protein, 10 aa, synthetic construct
ERWELLYFDY

SEQ ID NO: 633 - protein, 9 aa, synthetic construct
EKRELLFDY

SEQ ID NO: 634 - protein, 10 aa, synthetic construct
ERWELLYFDY

SEQ ID NO: 635 - protein, 9 aa, synthetic construct
ERVGAGFDY

SEQ ID NO: 636 - protein, 10 aa, synthetic construct
ERIRDLYFDY

SEQ ID NO: 637 - protein, 10 aa, synthetic construct
ERGKGAGMDV

SEQ ID NO: 638 - protein, 9 aa, synthetic construct
EYYYYGMDV

SEQ ID NO: 639 - protein, 10 aa, synthetic construct
EKRSRLYFDY

SEQ ID NO: 640 - protein, 10 aa, synthetic construct
ERWELLYFDY

SEQ ID NO: 641 - protein, 10 aa, synthetic construct
ERWNLLYFDY

SEQ ID NO: 642 - protein, 8 aa, synthetic construct
VTFDAFDI

SEQ ID NO: 643 - protein, 10 aa, synthetic construct
EKWELLFFDY

SEQ ID NO: 644 - protein, 10 aa, synthetic construct
ERISRLYFDY

SEQ ID NO: 645 - protein, 10 aa, synthetic construct
EDRYRLFFDY

SEQ ID NO: 646 - protein, 10 aa, synthetic construct
EKWDLLYFDY

SEQ ID NO: 647 - protein, 9 aa, synthetic construct
DRGELTLWY

SEQ ID NO: 648 - protein, 9 aa, synthetic construct
DRRELLLDY

SEQ ID NO: 649 - protein, 9 aa, synthetic construct
DRRELPLDY

SEQ ID NO: 650 - protein, 12 aa, synthetic construct
DGELGIGTYFDY

SEQ ID NO: 651 - protein, 10 aa, synthetic construct
ERWELLYFDY

SEQ ID NO: 652 - protein, 10 aa, synthetic construct
ERWELLYFDY

SEQ ID NO: 653 - protein, 9 aa, synthetic construct
DWRELLLDY

SEQ ID NO: 654 - protein, 10 aa, synthetic construct
ERKTSLPFDI

SEQ ID NO: 655 - protein, 9 aa, synthetic construct
DRRELLLDY

SEQ ID NO: 656 - protein, 8 aa, synthetic construct
DFNWTFDN

SEQ ID NO: 657 - protein, 10 aa, synthetic construct
EKWVLLFFDY

SEQ ID NO: 658 - protein, 8 aa, synthetic construct
DLNWAFDY

SEQ ID NO: 659 - protein, 8 aa, synthetic construct
DHNWSFDY

SEQ ID NO: 660 - protein, 10 aa, synthetic construct
EGGNSHGMDV

SEQ ID NO: 661 - protein, 9 aa, synthetic construct
ERRTGYFDY

SEQ ID NO: 662 - protein, 10 aa, synthetic construct
VPYSGSYFDY

SEQ ID NO: 663 - protein, 8 aa, synthetic construct
YRYNWNDY

SEQ ID NO: 664 - protein, 8 aa, synthetic construct
YRYNWNDY

SEQ ID NO: 665 - protein, 12 aa, synthetic construct
EGPNWGTHAFDI

SEQ ID NO: 666 - protein, 8 aa, synthetic construct
YRYNWNDY

SEQ ID NO: 667 - protein, 8 aa, synthetic construct
ERRNGFDY

SEQ ID NO: 668 - protein, 8 aa, synthetic construct
YRYNWNDY

SEQ ID NO: 669 - protein, 8 aa, synthetic construct
AVYNYFDQ

SEQ ID NO: 670 - protein, 12 aa, synthetic construct
EGPNWGTHAFDI

SEQ ID NO: 671 - protein, 9 aa, synthetic construct
ERRELAFDI

SEQ ID NO: 672 - protein, 9 aa, synthetic construct
ERRELSFDY

SEQ ID NO: 673 - protein, 12 aa, synthetic construct
EGPNWGTHAFDI

SEQ ID NO: 674 - protein, 8 aa, synthetic construct
YRYNWNDY

SEQ ID NO: 675 - protein, 9 aa, synthetic construct
ERRELSFDY

SEQ ID NO: 676 - protein, 8 aa, synthetic construct
VSYNSFDY

SEQ ID NO: 677 - protein, 8 aa, synthetic construct
AVYNYFDQ

SEQ ID NO: 678 - protein, 8 aa, synthetic construct
YRYNWNDY

SEQ ID NO: 679 - protein, 8 aa, synthetic construct
ERRNGFDY

SEQ ID NO: 680 - protein, 9 aa, synthetic construct
QGYLDAFDI

SEQ ID NO: 681 - protein, 8 aa, synthetic construct
ALYNYFDY

SEQ ID NO: 682 - protein, 8 aa, synthetic construct
YRYNWNDY

SEQ ID NO: 683 - protein, 9 aa, synthetic construct
ERRDLAFDI

SEQ ID NO: 684 - protein, 12 aa, synthetic construct
EGPNWGTHAFDI

SEQ ID NO: 685 - protein, 9 aa, synthetic construct
ERRELSFDY

SEQ ID NO: 686 - protein, 9 aa, synthetic construct
ERRELSFDY

SEQ ID NO: 687 - protein, 9 aa, synthetic construct
SWGYYVMDV

SEQ ID NO: 688 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGGGTKLTVL

SEQ ID NO: 689 - protein, 106 aa, synthetic construct
SYELTQPPSVSVSPGQTASITCSGDKLGNKYACWYQQKPGQSPVLVIYQDSKRPSGIPERFSGSNSGNTATLTISGTQAMDEADYYCQAWDISTVVFGGGTKLTVL

SEQ ID NO: 690 - protein, 106 aa, synthetic construct
SYELTQPPSVSVSPGQTASITCSGDKLRDKYACWYQQKPGQSPVLVIYQDSKRPSGIPERFSGSNSGNTATLTISGTQAMDEADYYCQAWDSSTVVFGGGTKLTVL

SEQ ID NO: 691 - protein, 106 aa, synthetic construct
SYELTQPPSVSVSPGQTASITCSGDKLGDKYAGWYQQKPGQSPVLVIYQDSKRPSGIPERFSGSNSGNTATLTISGTQAMDEADYYCQAWDSSTVVFGGGTKLTVL

SEQ ID NO: 692 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNHVFGGGTKLTVL

SEQ ID NO: 693 - protein, 109 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHSWVFGGGTKLTVL

SEQ ID NO: 694 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDNSNRVFGGGTKLTVL

SEQ ID NO: 695 - protein, 109 aa, synthetic construct; identical to SEQ ID NO: 693

SEQ ID NO: 696 - protein, 109 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHCWVFGGGTKLTVL

SEQ ID NO: 697 - protein, 110 aa, synthetic construct
NFMLTQPHSVSESPGKTVTISCTRSSGSIASNYVQWYQQRPGSSPTTMIYEDNQRPSGVPDRFSGSIDSSSNSASLTISGLKTEDEADYYCQSLDSSSVVFGGGTKLTVL

SEQ ID NO: 698 - protein, 110 aa, synthetic construct
NFMLTQPHSVSESPGKTVTIACTRSSGSIASNYVQWYQQRPGSSPTTVIYEDNQRPSGVPDRFSGSIDSSSNSASLTISGLKTEDEADYYCQSYDSSNQVFGGGTKLTVL

SEQ ID NO: 699 - protein, 110 aa, synthetic construct
NFMLTQPHSVSESPGKTVTISCTRSNGSITSNYVQWYQQRPGSSPTTVIYEDNQRPSGVPDRFSGSIDSSSNSASLTISGLKTEDEADYYCQSFDNSNRVFGEKTKLTVL

SEQ ID NO: 700 - protein, 107 aa, synthetic construct
DIQMTQSPSTLSASVGDRVTITCRASQSISNWLAWYQQKPGKAPNLLIYKASSLESGVPSRFSGSGSGTEFTLTISSLQPDDFANYYCQQYKSYSWTFGQGTKVEIK

SEQ ID NO: 701 - protein, 110 aa, synthetic construct; identical to SEQ ID NO: 688

SEQ ID NO: 702 - protein, 110 aa, synthetic construct; identical to SEQ ID NO: 688

SEQ ID NO: 703 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYNSYSWTFGQGTKVEIK

SEQ ID NO: 704 - protein, 110 aa, synthetic construct
NFMLTQPHSVSESPGKTVTISCTRSSGSIASKYVQWYQQRPGSSPTTVIYEDNQRPSGVPDRFSGSIDSSSNSASLTISGLKTEDEADYYCQSYDSNNRVFGGGTKLTVL

SEQ ID NO: 705 - protein, 112 aa, synthetic construct
DIVMTQTPLSSPVTPGQPASISCRSSQSLVHSDGNTYLNWLQQRPGQPPRLLIYKISDRFSGVPDRFSGSGAGTDFTLKIGRVEAEDVGVYYCMQSTQFPITFGQGTRLEIK

SEQ ID NO: 706 - protein, 110 aa, synthetic construct; identical to SEQ ID NO: 694

SEQ ID NO: 707 - protein, 110 aa, synthetic construct
NFMLTQPHSVSESPGKTVTISCTRSSGSIASNYVQWYQQRPGSSPTSVIYEDNQRPSGVPDRFSGSIDSSSNSASLTISGLKTEDEADYYCQSYDRSNRVFGGGTKLTVL

SEQ ID NO: 708 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTHSSGNHRVFGGGTKLTVL

SEQ ID NO: 709 - protein, 108 aa, synthetic construct
SYELTQPPSVSVSPGQTARITCSGDALPKQYTYWCQQKPGQAPVLVIYKDSERPSGIPERFSGSSSGTTVTLTISGVQAEDEADYYCQSADSSGTYRVFGGGTKLTVL

SEQ ID NO: 710 - protein, 110 aa, synthetic construct
NFMLTQPHSVSESPGKTVTISCTRSSGSIASNYVQWYQQRPGSSPTTVIYEDNHRPSGVPDRFSGSIDSSSNSASLTISGLKTEDEADYFCQSFDSSNRVFGGGTKLTVL

SEQ ID NO: 711 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFYSSNRVFGGGTELTVL

SEQ ID NO: 712 - protein, 110 aa, synthetic construct; identical to SEQ ID NO: 710

SEQ ID NO: 713 - protein, 110 aa, synthetic construct; identical to SEQ ID NO: 710

SEQ ID NO: 714 - protein, 110 aa, synthetic construct
QTVVTQEPSFSVSPGGTVTLTCGLNSGSVSTSYYPSWYQQTPGQTPRTLIYSTNTRSSGVPDRFSGSILGNKAALTITGAQADDESDYYCVLYMGSGIWVFGGGTKLTVL

SEQ ID NO: 715 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDSSNVVFGGGTKLTVL

SEQ ID NO: 716 - protein, 110 aa, synthetic construct
QSVLTQPPSASGTPGQRVTISCSGSSSNIGSNYVYWYQQLPGTAPKLLIYSNNQRPSGVPDRFSGSKSGTSASLAISGLRSEDEADYYCEAWDDSLSGPVFGGGTKLTVL

SEQ ID NO: 717 - protein, 110 aa, synthetic construct
NFMLTQPHSVSESPGKTVTISCTRSSGSIASNYVQWYQHRPGSSPTTVIYEDDQRPSGIPDRFSGSIDSSSNSASLTISGLKTEDEADYYCQSYNSSNQVFGGGTKLTVL

SEQ ID NO: 718 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGFYYCMQPLQTPYTFGQGTKLEIK

SEQ ID NO: 719 - protein, 106 aa, synthetic construct
SYELTQPPSVSVSPGQTASITCSGDKLGYKYASWYQQKPGQSPVLVIYQDSKRPSGIPERFSGSNSGNTATLTISGTQAMDEADYYCQAWDSSTVVFGGGTKLTVL

SEQ ID NO: 720 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYYNSNRVFGGGTKLTVL

SEQ ID NO: 721 - protein, 110 aa, synthetic construct; identical to SEQ ID NO: 688

SEQ ID NO: 722 - protein, 109 aa, synthetic construct; identical to SEQ ID NO: 696

SEQ ID NO: 723 - protein, 107 aa, synthetic construct
DIQMTQSPSTLSASVGDRVTITCRASQSISSWLAWFQQKTGKAPKLLIYKASSLESGVPSRFSGSGSGTEFTLTISSLQPDDFATYYCQQYISYSWTFGQGTKVEIK

SEQ ID NO: 724 - protein, 106 aa, synthetic construct
DIQMTQSPSTLSASVGDRVTITCRASQSISSWLAWYQQKPGKAPKLLIHKASSLESGVPSRFSGSGSGTEFTLTISSLQPDDFATYYCQQYNSYWTFGQGTKVEIK

SEQ ID NO: 725 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDETDYNCQSYDSSLSGYVFGTGTKVTVL

SEQ ID NO: 726 - protein, 112 aa, synthetic construct
DVVMTQSPLSLPVTLGQPASISCRSSQSLVYSDGNTYLTWFQQRPGQSPRRLIYKVSNRDSGVPDRFSGSGSGTDFTLKISRVEAEDVEIYYCMQGTHWPLTFGGGTKVEIK

SEQ ID NO: 727 - protein, 110 aa, synthetic construct
QSVLTQPPSASGTPGQRVTISCSGSSSNIGSNTVNWYQQFPGTAPKLLIYSNNQRPSGVPDRFSGSKSGTSASLAISGLQSEDEADYYCAPWDDSLNGVVFGGGTKLTVL

SEQ ID NO: 728 - protein, 109 aa, synthetic construct; identical to SEQ ID NO: 696

SEQ ID NO: 729 - protein, 108 aa, synthetic construct
SYELTQPPSVSVSPGQTARITCSGDALPKQYTYWCQQKPGQAPILVIYKDSERPSGIPERFSGSSSGTTVTLTISGVQADDEADYYCQSADSSGTYRVFGGGTKLTVL

SEQ ID NO: 730 - protein, 111 aa, synthetic construct
QSALTQPASVSGSPGQSITISCTGTSSDVGSYNLVSWYQQHPGKAPKLMIYEDIRRPSGVSNRFSASKSGDTASLTISGLQAEDEADYYCCSYAGSTTFAIFGGGTKLTVL

SEQ ID NO: 731 - protein, 117 aa, synthetic construct
QPVLTQPSSHSASSGASVRLTCMLSSGFSVGDFWIRWYQQKPGNPPRYLLFYHSDSNKGQGSGVPSRFSGSNDASANAGILRISGLQPEDEADYYCGTWHSNSKTWVFGGGTKLTVL

SEQ ID NO: 732 - protein, 107 aa, synthetic construct
DIQMTQSPSTLSASVGDRVTITCRASQSISSWLAWYQQKPGKAPNLLIYKASSLESGVPSRFSGSGSGTEFTLTISSLQPDDFATYYCQQYKSYSWTIGQGTKVEIK

SEQ ID NO: 733 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQCYDRSNRVFGGGTKLTVL

SEQ ID NO: 734 - protein, 110 aa, synthetic construct
NFMLTQPHSVSESPGKTVTISCTRSSGSIASKYVQWYQQRPGSSPTTVIYEDNQRPSGVPDRFSGSIDSSSNSASLTISGLKTEDEADYYCQSYYSNNRVFGGGTKLTVL

SEQ ID NO: 735 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDSSNRVFGGGTKLTVL

SEQ ID NO: 736 - protein, 110 aa, synthetic construct
NFMLTQPHSVSESPGKTVTISCTRSSGSIASNYVQWYQQRPGSSPTTVIYEDNQRSSGVPDRFSGSIDSSSNSASLTISGLKTEDEADYYCQCYDRSNRVFGGGTKLTVL

SEQ ID NO: 737 - protein, 110 aa, synthetic construct
NFMLTQPHSVSESPGKTVTISCTRSSGSIASNYVQWYQQRPGISPTTVIYEDNQRPSGVPDRFSGSIDSSSNSASLTISGLKTEDEADYYCQSFDTSNVVFGGGTKLTVL

SEQ ID NO: 738 - protein, 110 aa, synthetic construct; identical to SEQ ID NO: 688

SEQ ID NO: 739 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHRVFGGGTKLTVL

SEQ ID NO: 740 - protein, 110 aa, synthetic construct; identical to SEQ ID NO: 688

SEQ ID NO: 741 - protein, 110 aa, synthetic construct
NSMLTQPHSVSESPGKTVTISCTRSSGSIASYYVQWYQQRPGSSPTTVIYEDNQRPSGVPDRFSGSIDSSSNSASLTISGLKTEDEADYYCQSFDSSNRVFGGGTKLTVL

SEQ ID NO: 742 - protein, 110 aa, synthetic construct
NFMLTQPHSVSESPGKTVTISCTRSSGSIASNYVQWYQQRPGSSPTTVIYEDNQRPSGVPDRFSGSIDRSSNSASLTISGLKTEDEADYYCQSYDSSNRVFGGGTKLTVL

SEQ ID NO: 743 - protein, 109 aa, synthetic construct
FMLTQPHSVSESPGKTVTISCTRSSGSIASNYVQWYQQRPGSSPTTVIYEDNQRPSGVPDRFSGSIDSSSNSASLTISGLQTEDEADYYCQSYDSSNRVFGGGTKLTVL

SEQ ID NO: 744 - protein, 117 aa, synthetic construct
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKTQVFGGGTKLTVL

SEQ ID NO: 745 - protein, 110 aa, synthetic construct
NFMLTQPHSVSESPGKTVTISCTRSSGSIASNFVQWYQQRPGSSPTTVIYEDNQRPSGVPDRFSGSIDSSSNSASLTISGLKTEDEADYYCQSFDSSNRVFGGGTKLTVL

SEQ ID NO: 746 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLQTEDEADYYCQSYDNSIRVFGGGTKLTVL

SEQ ID NO: 747 - protein, 110 aa, synthetic construct; identical to SEQ ID NO: 742

SEQ ID NO: 748 - protein, 109 aa, synthetic construct
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAYVFGTGTKVTVL

SEQ ID NO: 749 - protein, 110 aa, synthetic construct
NFMLTQPHSVSESPGKTVAISCTRSSGSIASNYVQWYQQRPGSSPTAVIYEDNQRPSGVPDRFSGSIDSSSNSAFLTISGLKTEDEADYYCQSYDSSNRVFGGGTKLTVL

SEQ ID NO: 750 - protein, 110 aa, synthetic construct
NFMLTQPHSVSESPGKTVTISCTRTSGSIASNYVQWYQQRPGSSPTTVIYEDNQRPSGVPDRFSGSIDSSSNSASLTISGLKTEDEAAYYCQSYDRSNRVFGGGTKLTVL

SEQ ID NO: 751 - protein, 110 aa, synthetic construct; identical to SEQ ID NO: 688

SEQ ID NO: 752 - protein, 110 aa, synthetic construct
QSVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGVVFGGGTKLTVL

SEQ ID NO: 753 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEGDYYCQSYDRSNRVFGGGTKLTVL

SEQ ID NO: 754 - protein, 110 aa, synthetic construct
NFMLTQPHSVSESPGKTVTISCTRSSGSIASNYVQWYRQRPGSSPTTVIYEDNQRPSGVPDRFSGAIDSSSTSASLTISGLKTEDEADYYCQSYDSSNRVFGGGTKLTVL

SEQ ID NO: 755 - protein, 108 aa, synthetic construct
SYELTQPPSVSVSPGQTARITCSGDALPKQYAYWYQQKPGQAPVLVIYKDSERPSGIPERFSGSSSGTTVTLTISGVQAEDEADYYCQSADSSGTYVVFGGGTKLTVL

SEQ ID NO: 756 - protein, 110 aa, synthetic construct; identical to SEQ ID NO: 688

SEQ ID NO: 757 - protein, 110 aa, synthetic construct; identical to SEQ ID NO: 688

SEQ ID NO: 758 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDRSNRVFGGGTKLTVL

SEQ ID NO: 759 - protein, 110 aa, synthetic construct; identical to SEQ ID NO: 688

SEQ ID NO: 760 - protein, 110 aa, synthetic construct
NFMLTQPHSVSESPGKTVTISCTRSSGSIASNYVQWYQQRPGSSPTTVIYEDNRRPSGVPDRFSGSIDSSSNSASLTISGLKTEDEADYYCQSYDNSNRVFGGGTTLTVL

SEQ ID NO: 761 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHWVFGGGTKLTVL

SEQ ID NO: 762 - protein, 109 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHNWVFGGGTKLTVL

SEQ ID NO: 763 - protein, 108 aa, synthetic construct; identical to SEQ ID NO: 761

SEQ ID NO: 764 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKLTVL

SEQ ID NO: 765 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDGSNRVFGGGTKLTVL

SEQ ID NO: 766 - protein, 110 aa, synthetic construct
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGIWVFGGGTKLTVL

SEQ ID NO: 767 - protein, 109 aa, synthetic construct; identical to SEQ ID NO: 693

SEQ ID NO: 768 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEAYYYCQSFDSSNRVFGGGTKLTVL

SEQ ID NO: 769 - protein, 109 aa, synthetic construct; identical to SEQ ID NO: 693

SEQ ID NO: 770 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSSDRSNVVFGGGTKLTVL

SEQ ID NO: 771 - protein, 110 aa, synthetic construct
NFMLTQPHSVSESPGKTVTISCTRSSGSIASNYVQWYQQRPGSSPTTMIYEDTQRPSGVPDRFSGSIDSSSNSASLTISGLKTEDEADYYCQSYDSSNRVFGGGTKLTVL

SEQ ID NO: 772 - protein, 110 aa, synthetic construct
NFMLTQPHSVSESPGKTVTISCTRSSGSIASNYVQWYQQRPGSSPTTVIYEDNHRPSGVPDRFSGSIDSSSNSASLTIAGLKTEDEADYYCQSCDSSNVVFGGGTKLTVL

SEQ ID NO: 773 - protein, 110 aa, synthetic construct
NFMLTQPLSVSESPGKTLTISCTRNSGSIASNYVQWYRQRPGSSPTTVIYEDKQRPSGVPDRFSGSIDSSSNSASLTISGLKAEDEADYYCQSCDSSNVVFGGGTTLTVL

SEQ ID NO: 774 - protein, 110 aa, synthetic construct; identical to SEQ ID NO: 688

SEQ ID NO: 775 - protein, 107 aa, synthetic construct
DIQMTQSPSTLSASVGDRVTITCRASQSISSWLAWYQQKPGKAPNLLIYKASSLESGVPSRFSGSGSGTEFTLTISSLQPDDFATYYCQQYNSYSWTFGQGTKVEIK

SEQ ID NO: 776 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLAPEDFAVYYCQQYGSSRWSFGQGTKVEIK

SEQ ID NO: 777 - protein, 110 aa, synthetic construct; identical to SEQ ID NO: 688

SEQ ID NO: 778 - protein, 106 aa, synthetic construct; identical to SEQ ID NO: 689

SEQ ID NO: 779 - protein, 106 aa, synthetic construct; identical to SEQ ID NO: 690

SEQ ID NO: 780 - protein, 106 aa, synthetic construct; identical to SEQ ID NO: 691

SEQ ID NO: 781 - protein, 110 aa, synthetic construct; identical to SEQ ID NO: 692

SEQ ID NO: 782 - protein, 109 aa, synthetic construct; identical to SEQ ID NO: 693

SEQ ID NO: 783 - protein, 110 aa, synthetic construct; identical to SEQ ID NO: 694

SEQ ID NO: 784 - protein, 109 aa, synthetic construct; identical to SEQ ID NO: 693

SEQ ID NO: 785 - protein, 109 aa, synthetic construct; identical to SEQ ID NO: 696

SEQ ID NO: 786 - protein, 110 aa, synthetic construct; identical to SEQ ID NO: 697

SEQ ID NO: 787 - protein, 110 aa, synthetic construct; identical to SEQ ID NO: 698

SEQ ID NO: 788 - protein, 110 aa, synthetic construct; identical to SEQ ID NO: 699

SEQ ID NO: 789 - protein, 107 aa, synthetic construct; identical to SEQ ID NO: 700

SEQ ID NO: 790 - protein, 110 aa, synthetic construct; identical to SEQ ID NO: 688

SEQ ID NO: 791 - protein, 110 aa, synthetic construct; identical to SEQ ID NO: 688

SEQ ID NO: 792 - protein, 107 aa, synthetic construct; identical to SEQ ID NO: 703

SEQ ID NO: 793 - protein, 110 aa, synthetic construct; identical to SEQ ID NO: 704

SEQ ID NO: 794 - protein, 112 aa, synthetic construct; identical to SEQ ID NO: 705

SEQ ID NO: 795 - protein, 110 aa, synthetic construct; identical to SEQ ID NO: 694

SEQ ID NO: 796 - protein, 110 aa, synthetic construct; identical to SEQ ID NO: 707

SEQ ID NO: 797 - protein, 108 aa, synthetic construct; identical to SEQ ID NO: 708

SEQ ID NO: 798 - protein, 108 aa, synthetic construct; identical to SEQ ID NO: 709

SEQ ID NO: 799 - protein, 110 aa, synthetic construct; identical to SEQ ID NO: 710

SEQ ID NO: 800 - protein, 110 aa, synthetic construct; identical to SEQ ID NO: 711

SEQ ID NO: 801 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 802 - protein, 106 aa, synthetic construct
SYELTQPPSVSVSPGQTASITCSGDKLGNKYACWYQQKPGQSPVLVIYQDSKRPSGIPERFSGSNSGNTATLTISGTQAMDEADYYCQAWDISTVVFGCGTKLTVL

SEQ ID NO: 803 - protein, 106 aa, synthetic construct
SYELTQPPSVSVSPGQTASITCSGDKLRDKYACWYQQKPGQSPVLVIYQDSKRPSGIPERFSGSNSGNTATLTISGTQAMDEADYYCQAWDSSTVVFGCGTKLTVL

SEQ ID NO: 804 - protein, 106 aa, synthetic construct
SYELTQPPSVSVSPGQTASITCSGDKLGDKYAGWYQQKPGQSPVLVIYQDSKRPSGIPERFSGSNSGNTATLTISGTQAMDEADYYCQAWDSSTVVFGCGTKLTVL

SEQ ID NO: 805 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNHVFGCGTKLTVL

SEQ ID NO: 806 - protein, 109 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHSWVFGCGTKLTVL

SEQ ID NO: 807 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDNSNRVFGCGTKLTVL

SEQ ID NO: 808 - protein, 109 aa, synthetic construct; identical to SEQ ID NO: 806

SEQ ID NO: 809 - protein, 109 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHCWVFGCGTKLTVL

SEQ ID NO: 810 - protein, 110 aa, synthetic construct
NFMLTQPHSVSESPGKTVTISCTRSSGSIASNYVQWYQQRPGSSPTTMIYEDNQRPSGVPDRFSGSIDSSSNSASLTISGLKTEDEADYYCQSLDSSSVVFGCGTKLTVL

SEQ ID NO: 811 - protein, 110 aa, synthetic construct
NFMLTQPHSVSESPGKTVTIACTRSSGSIASNYVQWYQQRPGSSPTTVIYEDNQRPSGVPDRFSGSIDSSSNSASLTISGLKTEDEADYYCQSYDSSNQVFGCGTKLTVL

SEQ ID NO: 812 - protein, 110 aa, synthetic construct
NFMLTQPHSVSESPGKTVTISCTRSNGSITSNYVQWYQQRPGSSPTTVIYEDNQRPSGVPDRFSGSIDSSSNSASLTISGLKTEDEADYYCQSFDNSNRVFGCKTKLTVL

SEQ ID NO: 813 - protein, 107 aa, synthetic construct
DIQMTQSPSTLSASVGDRVTITCRASQSISNWLAWYQQKPGKAPNLLIYKASSLESGVPSRFSGSGSGTEFTLTISSLQPDDFANYYCQQYKSYSWTFGCGTKVEIK

SEQ ID NO: 814 - protein, 110 aa, synthetic construct; identical to SEQ ID NO: 801

SEQ ID NO: 815 - protein, 110 aa, synthetic construct; identical to SEQ ID NO: 801

SEQ ID NO: 816 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYNSYSWTFGCGTKVEIK

SEQ ID NO: 817 - protein, 110 aa, synthetic construct
NFMLTQPHSVSESPGKTVTISCTRSSGSIASKYVQWYQQRPGSSPTTVIYEDNQRPSGVPDRFSGSIDSSSNSASLTISGLKTEDEADYYCQSYDSNNRVFGCGTKLTVL

SEQ ID NO: 818 - protein, 112 aa, synthetic construct
DIVMTQTPLSSPVTPGQPASISCRSSQSLVHSDGNTYLNWLQQRPGQPPRLLIYKISDRFSGVPDRFSGSGAGTDFTLKIGRVEAEDVGVYYCMQSTQFPITFGCGTRLEIK

SEQ ID NO: 819 - protein, 110 aa, synthetic construct; identical to SEQ ID NO: 807

SEQ ID NO: 820 - protein, 110 aa, synthetic construct
NFMLTQPHSVSESPGKTVTISCTRSSGSIASNYVQWYQQRPGSSPTSVIYEDNQRPSGVPDRFSGSIDSSSNSASLTISGLKTEDEADYYCQSYDRSNRVFGCGTKLTVL

SEQ ID NO: 821 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTHSSGNHRVFGCGTKLTVL

SEQ ID NO: 822 - protein, 108 aa, synthetic construct
SYELTQPPSVSVSPGQTARITCSGDALPKQYTYWYQQKPGQAPVLVIYKDSERPSGIPERFSGSSSGTTVTLTISGVQAEDEADYYCQSADSSGTYRVFGCGTKLTVL

SEQ ID NO: 823 - protein, 110 aa, synthetic construct
NFMLTQPHSVSESPGKTVTISCTRSSGSIASNYVQWYQQRPGSSPTTVIYEDNHRPSGVPDRFSGSIDSSSNSASLTISGLKTEDEADYFCQSFDSSNRVFGCGTKLTVL

SEQ ID NO: 824 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFYSSNRVFGCGTELTVL

SEQ ID NO: 825 - protein, 110 aa, synthetic construct; identical to SEQ ID NO: 823

SEQ ID NO: 826 - protein, 110 aa, synthetic construct; identical to SEQ ID NO: 823

SEQ ID NO: 827 - protein, 110 aa, synthetic construct
QTVVTQEPSFSVSPGGTVTLTCGLNSGSVSTSYYPSWYQQTPGQTPRTLIYSTNTRSSGVPDRFSGSILGNKAALTITGAQADDESDYYCVLYMGSGIWVFGCGTKLTVL

SEQ ID NO: 828 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDSSNVVFGCGTKLTVL

SEQ ID NO: 829 - protein, 110 aa, synthetic construct
QSVLTQPPSASGTPGQRVTISCSGSSSNIGSNYVYWYQQLPGTAPKLLIYSNNQRPSGVPDRFSGSKSGTSASLAISGLRSEDEADYYCEAWDDSLSGPVFGCGTKLTVL

SEQ ID NO: 830 - protein, 110 aa, synthetic construct
NFMLTQPHSVSESPGKTVTISCTRSSGSIASNYVQWYQHRPGSSPTTVIYEDDQRPSGIPDRFSGSIDSSSNSASLTISGLKTEDEADYYCQSYNSSNQVFGCGTKLTVL

SEQ ID NO: 831 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGFYYCMQPLQTPYTFGCGTKLEIK

SEQ ID NO: 832 - protein, 106 aa, synthetic construct
SYELTQPPSVSVSPGQTASITCSGDKLGYKYASWYQQKPGQSPVLVIYQDSKRPSGIPERFSGSNSGNTATLTISGTQAMDEADYYCQAWDSSTVVFGCGTKLTVL

SEQ ID NO: 833 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYYNSNRVFGCGTKLTVL

SEQ ID NO: 834 - protein, 110 aa, synthetic construct; identical to SEQ ID NO: 801

SEQ ID NO: 835 - protein, 109 aa, synthetic construct; identical to SEQ ID NO: 809

SEQ ID NO: 836 - protein, 107 aa, synthetic construct
DIQMTQSPSTLSASVGDRVTITCRASQSISSWLAWFQQKTGKAPKLLIYKASSLESGVPSRFSGSGSGTEFTLTISSLQPDDFATYYCQQYISYSWTFGCGTKVEIK

SEQ ID NO: 837 - protein, 106 aa, synthetic construct
DIQMTQSPSTLSASVGDRVTITCRASQSISSWLAWYQQKPGKAPKLLIHKASSLESGVPSRFSGSGSGTEFTLTISSLQPDDFATYYCQQYNSYWTFGCGTKVEIK

SEQ ID NO: 838 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDETDYNCQSYDSSLSGYVFGCGTKVTVL

SEQ ID NO: 839 - protein, 112 aa, synthetic construct
DVVMTQSPLSLPVTLGQPASISCRSSQSLVYSDGNTYLTWFQQRPGQSPRRLIYKVSNRDSGVPDRFSGSGSGTDFTLKISRVEAEDVEIYYCMQGTHWPLTFGCGTKVEIK

SEQ ID NO: 840 - protein, 110 aa, synthetic construct
QSVLTQPPSASGTPGQRVTISCSGSSSNIGSNTVNWYQQFPGTAPKLLIYSNNQRPSGVPDRFSGSKSGTSASLAISGLQSEDEADYYCAPWDDSLNGVVFGCGTKLTVL

SEQ ID NO: 841 - protein, 109 aa, synthetic construct; identical to SEQ ID NO: 806

SEQ ID NO: 842 - protein, 108 aa, synthetic construct
SYELTQPPSVSVSPGQTARITCSGDALPKQYTYWCQQKPGQAPILVIYKDSERPSGIPERFSGSSSGTTVTLTISGVQADDEADYYCQSADSSGTYRVFGCGTKLTVL

SEQ ID NO: 843 - protein, 111 aa, synthetic construct
QSALTQPASVSGSPGQSITISCTGTSSDVGSYNLVSWYQQHPGKAPKLMIYEDIRRPSGVSNRFSASKSGDTASLTISGLQAEDEADYYCSSYAGSTTFAIFGCGTKLTVL

SEQ ID NO: 844 - protein, 117 aa, synthetic construct
QPVLTQPSSHSASSGASVRLTCMLSSGFSVGDFWIRWYQQKPGNPPRYLLFYHSDSNKGQGSGVPSRFSGSNDASANAGILRISGLQPEDEADYYCGTWHSNSKTWVFGCGTKLTVL

SEQ ID NO: 845 - protein, 107 aa, synthetic construct
DIQMTQSPSTLSASVGDRVTITCRASQSISSWLAWYQQKPGKAPNLLIYKASSLESGVPSRFSGSGSGTEFTLTISSLQPDDFATYYCQQYKSYSWTIGCGTKVEIK

SEQ ID NO: 846 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQCYDRSNRVFGCGTKLTVL

SEQ ID NO: 847 - protein, 110 aa, synthetic construct
NFMLTQPHSVSESPGKTVTISCTRSSGSIASKYVQWYQQRPGSSPTTVIYEDNQRPSGVPDRFSGSIDSSSNSASLTISGLKTEDEADYYCQSYYSNNRVFGCGTKLTVL

SEQ ID NO: 848 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDSSNRVFGCGTKLTVL

SEQ ID NO: 849 - protein, 110 aa, synthetic construct
NFMLTQPHSVSESPGKTVTISCTRSSGSIASNYVQWYQQRPGSSPTTVIYEDNQRSSGVPDRFSGSIDSSSNSASLTISGLKTEDEADYYCQSYDRSNRVFGCGTKLTVL

SEQ ID NO: 850 - protein, 110 aa, synthetic construct
NFMLTQPHSVSESPGKTVTISCTRSSGSIASNYVQWYQQRPGISPTTVIYEDNQRPSGVPDRFSGSIDSSSNSASLTISGLKTEDEADYYCQSFDTSNVVFGCGTKLTVL

SEQ ID NO: 851 - protein, 110 aa, synthetic construct; identical to SEQ ID NO: 801

SEQ ID NO: 852 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHRVFGCGTKLTVL

SEQ ID NO: 853 - protein, 110 aa, synthetic construct; identical to SEQ ID NO: 801

SEQ ID NO: 854 - protein, 110 aa, synthetic construct
NSMLTQPHSVSESPGKTVTISCTRSSGSIASYYVQWYQQRPGSSPTTVIYEDNQRPSGVPDRFSGSIDSSSNSASLTISGLKTEDEADYYCQSFDSSNRVFGCGTKLTVL

SEQ ID NO: 855 - protein, 110 aa, synthetic construct
NFMLTQPHSVSESPGKTVTISCTRSSGSIASNYVQWYQQRPGSSPTTVIYEDNQRPSGVPDRFSGSIDRSSNSASLTISGLKTEDEADYYCQSYDSSNRVFGCGTKLTVL

SEQ ID NO: 856 - protein, 109 aa, synthetic construct
FMLTQPHSVSESPGKTVTISCTRSSGSIASNYVQWYQQRPGSSPTTVIYEDNQRPSGVPDRFSGSIDSSSNSASLTISGLQTEDEADYYCQSYDSSNRVFGCGTKLTVL

SEQ ID NO: 857 - protein, 117 aa, synthetic construct
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKTQVFGCGTKLTVL

SEQ ID NO: 858 - protein, 110 aa, synthetic construct
NFMLTQPHSVSESPGKTVTISCTRSSGSIASNFVQWYQQRPGSSPTTVIYEDNQRPSGVPDRFSGSIDSSSNSASLTISGLKTEDEADYYCQSFDSSNRVFGCGTKLTVL

SEQ ID NO: 859 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLQTEDEADYYCQSYDNSIRVFGCGTKLTVL

SEQ ID NO: 860 - protein, 110 aa, synthetic construct; identical to SEQ ID NO: 855

SEQ ID NO: 861 - protein, 109 aa, synthetic construct
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAYVFGCGTKVTVL

SEQ ID NO: 862 - protein, 110 aa, synthetic construct
NFMLTQPHSVSESPGKTVAISCTRSSGSIASNYVQWYQQRPGSSPTAVIYEDNQRPSGVPDRFSGSIDSSSNSAFLTISGLKTEDEADYYCQSYDSSNRVFGCGTKLTVL

SEQ ID NO: 863 - protein, 110 aa, synthetic construct
NFMLTQPHSVSESPGKTVTISCTRTSGSIASNYVQWYQQRPGSSPTTVIYEDNQRPSGVPDRFSGSIDSSSNSASLTISGLKTEDEAAYYCQSYDRSNRVFGCGTKLTVL

SEQ ID NO: 864 - protein, 110 aa, synthetic construct; identical to SEQ ID NO: 801

SEQ ID NO: 865 - protein, 110 aa, synthetic construct
QSVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGVVFGCGTKLTVL

SEQ ID NO: 866 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEGDYYCQSYDRSNRVFGCGTKLTVL

SEQ ID NO: 867 - protein, 110 aa, synthetic construct
NFMLTQPHSVSESPGKTVTISCTRSSGSIASNYVQWYRQRPGSSPTTVIYEDNQRPSGVPDRFSGAIDSSSTSASLTISGLKTEDEADYYCQSYDSSNRVFGCGTKLTVL

SEQ ID NO: 868 - protein, 108 aa, synthetic construct
SYELTQPPSVSVSPGQTARITCSGDALPKQYAYWYQQKPGQAPVLVIYKDSERPSGIPERFSGSSSGTTVTLTISGVQAEDEADYYCQSADSSGTYVVFGCGTKLTVL

SEQ ID NO: 869 - protein, 110 aa, synthetic construct; identical to SEQ ID NO: 801

SEQ ID NO: 870 - protein, 110 aa, synthetic construct; identical to SEQ ID NO: 801

SEQ ID NO: 871 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDRSNRVFGCGTKLTVL

SEQ ID NO: 872 - protein, 110 aa, synthetic construct; identical to SEQ ID NO: 801

SEQ ID NO: 873 - protein, 110 aa, synthetic construct
NFMLTQPHSVSESPGKTVTISCTRSSGSIASNYVQWYQQRPGSSPTTVIYEDNRRPSGVPDRFSGSIDSSSNSASLTISGLKTEDEADYYCQSYDNSNRVFGCGTTLTVL

SEQ ID NO: 874 - protein, 108 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHWVFGCGTKLTVL

SEQ ID NO: 875 - protein, 109 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHNWVFGCGTKLTVL

SEQ ID NO: 876 - protein, 108 aa, synthetic construct; identical to SEQ ID NO: 874

SEQ ID NO: 877 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGCGTKLTVL

SEQ ID NO: 878 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSFDGSNRVFGCGTKLTVL

SEQ ID NO: 879 - protein, 110 aa, synthetic construct
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGIWVFGCGTKLTVL

SEQ ID NO: 880 - protein, 109 aa, synthetic construct; identical to SEQ ID NO: 806

SEQ ID NO: 881 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEAYYYCQSFDSSNRVFGCGTKLTVL

SEQ ID NO: 882 - protein, 109 aa, synthetic construct; identical to SEQ ID NO: 806

SEQ ID NO: 883 - protein, 110 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSSDRSNVVFGCGTKLTVL

SEQ ID NO: 884 - protein, 110 aa, synthetic construct
NFMLTQPHSVSESPGKTVTISCTRSSGSIASNYVQWYQQRPGSSPTTMIYEDTQRPSGVPDRFSGSIDSSSNSASLTISGLKTEDEADYYCQSYDSSNRVFGCGTKLTVL

SEQ ID NO: 885 - protein, 110 aa, synthetic construct
NFMLTQPHSVSESPGKTVTISCTRSSGSIASNYVQWYQQRPGSSPTTVIYEDNHRPSGVPDRFSGSIDSSSNSASLTIAGLKTEDEADYYCQSYDSSNVVFGCGTKLTVL

SEQ ID NO: 886 - protein, 110 aa, synthetic construct
NFMLTQPHSVSESPGKTVTISCTRSSGSIASNYVQWYQQRPGSSPTTVIYEDNHRPSGVPDRFSGSIDSSSNSASLTIAGLKTEDEADYYCQSSDSSNVVFGCGTKLTVL

SEQ ID NO: 887 - protein, 110 aa, synthetic construct
NFMLTQPLSVSESPGKTLTISCTRNSGSIASNYVQWYRQRPGSSPTTVIYEDKQRPSGVPDRFSGSIDSSSNSASLTISGLKAEDEADYYCQSCDSSNVVFGCGTTLTVL

SEQ ID NO: 888 - protein, 110 aa, synthetic construct; identical to SEQ ID NO: 801

SEQ ID NO: 889 - protein, 107 aa, synthetic construct
DIQMTQSPSTLSASVGDRVTITCRASQSISSWLAWYQQKPGKAPNLLIYKASSLESGVPSRFSGSGSGTEFTLTISSLQPDDFATYYCQQYNSYSWTFGCGTKVEIK

SEQ ID NO: 890 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLAPEDFAVYYCQQYGSSRWSFGCGTKVEIK

SEQ ID NO: 891 - protein, 110 aa, synthetic construct; identical to SEQ ID NO: 801

SEQ ID NO: 892 - protein, 106 aa, synthetic construct; identical to SEQ ID NO: 802

SEQ ID NO: 893 - protein, 106 aa, synthetic construct; identical to SEQ ID NO: 803

SEQ ID NO: 894 - protein, 106 aa, synthetic construct; identical to SEQ ID NO: 804

SEQ ID NO: 895 - protein, 110 aa, synthetic construct; identical to SEQ ID NO: 805

SEQ ID NO: 896 - protein, 109 aa, synthetic construct; identical to SEQ ID NO: 806

SEQ ID NO: 897 - protein, 110 aa, synthetic construct; identical to SEQ ID NO: 807

SEQ ID NO: 898 - protein, 109 aa, synthetic construct; identical to SEQ ID NO: 806

SEQ ID NO: 899 - protein, 109 aa, synthetic construct; identical to SEQ ID NO: 809

SEQ ID NO: 900 - protein, 110 aa, synthetic construct; identical to SEQ ID NO: 810

SEQ ID NO: 901 - protein, 110 aa, synthetic construct; identical to SEQ ID NO: 811

SEQ ID NO: 902 - protein, 110 aa, synthetic construct; identical to SEQ ID NO: 812

SEQ ID NO: 903 - protein, 107 aa, synthetic construct; identical to SEQ ID NO: 813

SEQ ID NO: 904 - protein, 110 aa, synthetic construct; identical to SEQ ID NO: 801

SEQ ID NO: 905 - protein, 110 aa, synthetic construct; identical to SEQ ID NO: 801

SEQ ID NO: 906 - protein, 107 aa, synthetic construct; identical to SEQ ID NO: 816

SEQ ID NO: 907 - protein, 110 aa, synthetic construct; identical to SEQ ID NO: 817

SEQ ID NO: 908 - protein, 112 aa, synthetic construct; identical to SEQ ID NO: 818

SEQ ID NO: 909 - protein, 110 aa, synthetic construct; identical to SEQ ID NO: 807

SEQ ID NO: 910 - protein, 110 aa, synthetic construct; identical to SEQ ID NO: 820

SEQ ID NO: 911 - protein, 108 aa, synthetic construct; identical to SEQ ID NO: 821

SEQ ID NO: 912 - protein, 108 aa, synthetic construct
SYELTQPPSVSVSPGQTARITCSGDALPKQYTYWCQQKPGQAPVLVIYKDSERPSGIPERFSGSSSGTTVTLTISGVQAEDEADYYCQSADSSGTYRVFGCGTKLTVL

SEQ ID NO: 913 - protein, 110 aa, synthetic construct; identical to SEQ ID NO: 823

SEQ ID NO: 914 - protein, 110 aa, synthetic construct; identical to SEQ ID NO: 824

SEQ ID NO: 915 - protein, 119 aa, synthetic construct
EVQLVESGGGLVKPGGSLRLSCAASGFTLSSYSMNWVRQAPGKGLEWVSSISRSGSYIYYADSVKGRFTISRDNAKNSLYLQMNSLRAEDTAVYYCAREKWELLYFDYWGQGTLVTVSS

SEQ ID NO: 916 - protein, 117 aa, synthetic construct
EVQLVESGGGLVKPGGSLRLSCAASGFTFSSYSMNWVRQAPGKGLEWVSSISRSTSYIYYADSVKGRFTISRDNAKNSLYLQMNSLRAEDTAVYYCARGSRDAFDIWGQGTMVTVSS

SEQ ID NO: 917 - protein, 119 aa, synthetic construct
EVQLVESGGGLVKPGGSLRLSCAASGFTFSSYSMNWVRQAPGKGLEWVSYISRSSNYIHYADSVKGRFTIFRDNAKNSLYLQMNSLRVEDTAVYYCARAERGSYAVDIWGQGTMVTVSS

SEQ ID NO: 918 - protein, 118 aa, synthetic construct
VQLVESGGGLVKPGGSLRLSCAASGFTFSSYNMNWVRQAPGKGLEWVSSISSSSSYIYYADSVKGRFTISRDNAKNSLYLQMNSLRAEDTAVFYCARAERGSYAFDIWGQGTMVTVSS

SEQ ID NO: 919 - protein, 117 aa, synthetic construct
EVQLVESGGGLVQPGGSLRLSCAASGFTFSRYSMNWVRQAPGKGLEWVSYISSSSSSIYYADSVKGRFTISRDNAKNSLYLQMNSLRDEDTAVYYCARDRRATLDYWGQGTLVTVSS

SEQ ID NO: 920 - protein, 118 aa, synthetic construct
EVQLVDSGGGLVKPGGSLRLSCAASGFTFSSYSMNWVRQAPGKGLEWVSSISSRGSYRNYADSVKGRFTISRDNAKNSLYLQMNSLRAEDTAVYYCARDRRELLLDCWGQGTLVTVSS

SEQ ID NO: 921 - protein, 118 aa, synthetic construct
EVQLVESGGGLVKPGGSLRLSCAASGFTFSRYSMNWVRQAPGKGLKWVSSISSSGSYKYYADSVKGRFTISRDNAKNSLYLQMNSLRAEDTAVYYCAREPPTLGMDYWGQGTLVTVSS

SEQ ID NO: 922 - protein, 118 aa, synthetic construct
EVQLVESGGGLVKPGGSLRLSCAASGFTFSSYSMNWVRQAPGKGLEWVSSISSSSSYIYYADSVKGRFTISRANAKNSLYLQMSSLRAEDTAVYYCARDRRELLLDYWGQGTLVTVSS

SEQ ID NO: 923 - protein, 118 aa, synthetic construct
EVQLVDSGGGLVKPGGSLRLSCAASGFTFSSYSMNWVRQAPGKGLEWVSSVNSRGSYIYYADSVKGRFTISRDNAKNSLYLQMNSLRADDTAVYYCARDRRELLLDYWGQGTLVTVSS

SEQ ID NO: 924 - protein, 122 aa, synthetic construct
QVQLQQSGPGLVKPSQTLSLTCAISGDSVSCNSVAWNWIRQSPSRGLEWLGRTYYRSKWHNEYAVSVKSRINIKPDTSKNQVSLQLKSVTPEDTAVYYCARERKWELFLDYWGQGTLVTVSS

SEQ ID NO: 925 - protein, 121 aa, synthetic construct
QVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARLGYYPYWFFDVWGTGTTVTVSS

SEQ ID NO: 926 - protein, 118 aa, synthetic construct
EVQLVESGGGLVKPGGSLRLSCAASGFTFSSYTMNWVRQAPGKGLEWVSSISRSSSYKYYADSVKGRFTISRDNAKNSLYLQMNSLRAEDTAVYYCAREPPTLGMDYWGQGTLVIVSS

SEQ ID NO: 927 - protein, 118 aa, synthetic construct
EVQLVESGGGLVQPGGSLRLSCAASGFTFSNYWMSWVRQAPGKGLEWVANIKEDGSEKYYVDSVKGRFTISRDNAKNSLYLQMNSLTAEDTSVYYCARERRTGYFDYWGQGTLVTVSS

SEQ ID NO: 928 - protein, 119 aa, synthetic construct
EVQLVESGGGLVKPGGSLRLSCAASGFTCSSYSMTWVRQAPGKGLEWVSSISRRSSYIYYADSVKGRFTISRDNAKNSLYLQLNSLRAEDTAVYYCARERWELLYFDYWGQGTLVTVSS

SEQ ID NO: 929 - protein, 119 aa, synthetic construct
EVQLVESGGGLVKPGGSLRLSCAASGFNCSSYSMNWVRQAPGKGLEWVSSISRSSNYIYYADSVKGRFSISRDDAKNSLYLQMNSLRAEDTAVYYCARERWIQLYSDYWGQGILVTVSS

SEQ ID NO: 930 - protein, 118 aa, synthetic construct
EVQLVESGGGLVQPGGSLRLSCAASGFTFSRYWMTWVRQAPGKGLEWVANIKEDGSEKYCVDSVKGRFTISRDNAKNSLYLQLNSLRAEDTAVYYCARERRTGYFDYWGQGTLVTVSS

SEQ ID NO: 931 - protein, 119 aa, synthetic construct
EVQLVESGGGLVQPGGSLRLSCAASGFTFSRYSINWVRQAPGKGLEWVSYISSSSSTIYYADSVKGRFTISRDNAKNSLYLQMNSLRDEDTAVYYCARERWELLYFDYWGQGTLVTVSS

SEQ ID NO: 932 - protein, 115 aa, synthetic construct
EVQLVESGEGLVQPGGSLRLSCAASGFTFSSYAMHWVRQAPGKGLEYVSAISSNGGRTYYADSVKGRFTISRDNSKNTLYLQMGSLRAEDMAVYYCARGGSLDYWGQGTLVTVSS

SEQ ID NO: 933 - protein, 119 aa, synthetic construct
EVQLVESGGGLVKPGGSLRLSCAASGFTLSSYSMNWVRQAPGKGLEWVSSISRSSSYIYYADSVKGRFTISRDNAKNSLYLQMNSLRAEDTAVYYCARERWELLYFDYWGQGTLVTVSS

SEQ ID NO: 934 - protein, 118 aa, synthetic construct
EVQLVESGGGLVKPGGSLRLSCAASGLTFSTYSMNWVRQAPGKGLEWVSSISSSGSYVYYADSVKGRFTMSRDNAKNSLYLQMNSLRAEDTAVYYCARERRELLFDYWGQGTLVTVSS

SEQ ID NO: 935 - protein, 119 aa, synthetic construct
EVQLVESGGGLVKPGGSLRLSCAASGFTFSSYSMNWVRQAPGKGLEWVSSISRSSSYIYYADSVKGRFTLSRDNAKNSLYLQMNSLRGEDTAIYYCGRDGKGAIFLDYWGQGTLVTVSS

SEQ ID NO: 936 - protein, 119 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCARDGPVDYGMDVWGQGTTVTVSS

SEQ ID NO: 937 - protein, 118 aa, synthetic construct
EVHLVESGGGLVKPGGSLRLSCAASGFTFSSYSMNWVRQAPGKGLEWVSSISSSSTYIYYADSVKGRFTISRDNAKNSLYLQMNSLRAEDTAVYYCAREPRTLGMDYWGQGILVTVSS

SEQ ID NO: 938 - protein, 118 aa, synthetic construct
EVQLVESGGGLVKPGGSLRLSCVASGFTFSSYTMNWVRQAPGKGLEWVSSISRSGSYKYNADSVKGRFTISRDNAKNSLYLQMNSLRVEDTAVYYCAREPPTLGMDYWGQGTLVTVSS

SEQ ID NO: 939 - protein, 118 aa, synthetic construct; identical to SEQ ID NO: 937

SEQ ID NO: 940 - protein, 118 aa, synthetic construct; identical to SEQ ID NO: 937

SEQ ID NO: 941 - protein, 118 aa, synthetic construct
QVQLVQSGAEVKKPGASLKVSCKASGYTFTSYYMHWVRQAPGQGLEWMGIINPSNGNTISAQKFQGRVTMTRDTSTSTVYMEMSSLRSEDTAVYYCVRCWGYYVMDVWGQGTTVTVSS

SEQ ID NO: 942 - protein, 122 aa, synthetic construct
QVQLQQSGPGLVKPSQTLSLTCAISGDSVSSNSVAWNWIRQSPSRGLEWLGRTYYRSKWYNECAGSVKSRITINPDTSKNQFSLQLNSVTPEDTAIYYCARERKWELFLDYWGQGTLVTVSS

SEQ ID NO: 943 - protein, 118 aa, synthetic construct
EVQLVQSGAEVKKPGESLKISCKGSGYSFISYWIAWVRQMPGKGLEWMGIIYPGDSETRYRPSFQGQVTISADKSINTAYLQWSSLKAADTAMYYCARQGAYDGFDIWGQGTMVTVSS

SEQ ID NO: 944 - protein, 117 aa, synthetic construct
QVQLQESGPGLVKPSETLSLTCTVSGGSINSYYWSWIRQSPGKGLEWIGHIYYSGRTKYNPSLKSRVTISVDTSKNQFSLKLSSVTAADTAVYYSARENRNGAFDIWGQGTVVTVSS

SEQ ID NO: 945 - protein, 121 aa, synthetic construct
EVQLVQSGAEVKKPGESLKISCKGSGYSFTNYWIGWVRQMPGKGLEWMGIIYPADSDTTYSPSFQGQVTISADKSITTAYLQWSSLKASDTAMYYCARHGGSYILDAFDIWGQGTMVTVSS

SEQ ID NO: 946 - protein, 119 aa, synthetic construct
EVQLVESGGGLVKPGGSLRLSCEASGFTFSSYNMNWVRQAPGKGLEWVSSISSSSSYIYYTDSVKGRFTISRDNAKNSLYLQMNSLRAEDTAVYYCARAERGSYAFDFWGQGTMVTVSS

SEQ ID NO: 947 - protein, 118 aa, synthetic construct
EVQLVESGGGLVKPGGSLRLSCAASGFTFSSYSMNWVRQAPGKGLEWVSSISRSSSYIYYADSVKGRFTISRDNAKNSLYLQMNSLRAEDTAVYYCAREPPTLGMDYWGQGTLVTVSS

SEQ ID NO: 948 - protein, 118 aa, synthetic construct
EVHLVESGGGLVKPGGSLRLSCAASGFTFSRYSMNWVRQAPGKGLEWVSSISRSSSYIYYADSVKGRFTISRDNAKNSLYLQMNSLRAEDTAVYYCAREPRTLGMDYWGQGILVTVSS

SEQ ID NO: 949 - protein, 118 aa, synthetic construct
EVQLVESGGGLVKPGGSLRLSCAASGFTFSSYSMNWVRQAPGKGLEWVSSVNSRGSYIYYADSVKGRFTISRDNAKDSLYLQMNSLRADDTAVYYCARDRRELLLDYWGQGTLVTVSS

SEQ ID NO: 950 - protein, 118 aa, synthetic construct
EVQLVESGGGLVQRGGSLRLSCAASGFTFSNYWMSWVRQAPGKGLEWVANIKEDGSEKYCVDSVKGRFTISRDNAKHSLYLQMNSLRAEDTAVYYCARERRTGYFDYWGQGTLVTVSS

SEQ ID NO: 951 - protein, 119 aa, synthetic construct
EVQLVESGGGLVQPGGSLRLSCAASGFSFSSYSMNWVRQAPGKGLEWVSHIRSSTYSIYYADSVMGRFTISRDNAKNSLYLQMNSLRDEDTAVYFCARAKTGGDAFDIWGQGTMVTVSS

SEQ ID NO: 952 - protein, 118 aa, synthetic construct
QGQLQQWGAGLLKPSETLSLTCAVYGGSFSGYYWSWIRLPPGKGLEWIGEINHSGNTNYNPSLKSRVTISVDTSKNQFSLQLSSVTAADTAVYYCAKEVEIWGYFDYWGQGTLVTVSS

SEQ ID NO: 953 - protein, 122 aa, synthetic construct
EVQLLESGGGLVQPGGSLRLSCAASGFTFSYYAMSWVRQAPGKGLEWVSAISGSGGSTYYADSVKGRFTISRDNSKNTLYLQMNSLRAEDTAVYYCAKEGIVGARAEYFQHWGQGTLVTVSS

SEQ ID NO: 954 - protein, 122 aa, synthetic construct; identical to SEQ ID NO: 953

SEQ ID NO: 955 - protein, 118 aa, synthetic construct; identical to SEQ ID NO: 949

SEQ ID NO: 956 - protein, 119 aa, synthetic construct
EVQLVESGGGLVQPGGSLRLSCAASGFTFKSYSINWVRQAPGKGLEWVSYISSSSSTIYYADSVKGRFTISRDNAKNSLYLQMNSLRDEDTAVYYCARDGPVDYGMDVWGQGTTVTVSS

SEQ ID NO: 957 - protein, 118 aa, synthetic construct
EVQLVQSGAEVKKPGESLKISCKGSEYSFTTYWIAWVRQMPGKGLEWMGIIYPGDSDTTYSPSFQGQVTISADKSISIAFLQWSSLKASDTAMYYCARQGELDAFDIWGQGTMVTVSS

SEQ ID NO: 958 - protein, 114 aa, synthetic construct
EVQLVESGGGLVQPGGSLRLSCAASGFTFSTYWMSWVRQAPGKGLEWVANIKQDGSEKYYVDSVKGRFTISRDNAKNSLYLQMNSLRAEDTAVYYCATGSFDYWGQGTLVTVSS

SEQ ID NO: 959 - protein, 118 aa, synthetic construct
EVQLVESGGGLVQPGGSLRLSCAASGFTFSSYWMSWVRQAPGKGLEWVANIKEDGSEKYYVDSVKGRFTISRDNAKNSLYLQMNSLRAEDTAVYYCARERRTGYLDYWGQGTLVTVSS

SEQ ID NO: 960 - protein, 118 aa, synthetic construct
EVQLVESGGGLVKPGGSLRLSCSASGFTFSTYTMNWVRQAPGKGLDWVSSISKSSTYIYYADSVKGRFTISRDNAKNSLYLQMNSLRAEDTAVYYCARERRTLAFDIWGQGTMVTVSS

SEQ ID NO: 961 - protein, 119 aa, synthetic construct
EVQLVESGGGLVQPGGSLRLSCAASGFTFSRYSMNWVRQAPGKGLEWVSYISRSSTTIYYADSVKGRFTISRDNAKNSLYLQMNSLRDEDAAVYYCARERWELLYFDYWGQGTLVTVSS

SEQ ID NO: 962 - protein, 118 aa, synthetic construct
EVQLVESGGGLVKPGGSLRLSCAASGFTFSSYTMNWVRQAPGKGLEWVSSISGSGSYIYYADSVKGRFTISRDNAKNSLYLQMNSLRAEDTAVYYCAREPPTLGMDYWGQGTLVTVSS

SEQ ID NO: 963 - protein, 118 aa, synthetic construct
EVQLVESGGGLVKPGGSLRLSCSASGFTFSTYTMNWVRQAPGKGLEWVSSISKSSSYIYYTDSVKGRFTISRDNAKNSLYLQMNSLRAEDTAVYYCARERRTLAFDIWGQGTMVTVSS

SEQ ID NO: 964 - protein, 122 aa, synthetic construct
QVQLQQSGPGLVKPSQTLSLTCAISGDSVSSNSVAWNWIRQSPSRGLEWLGRTYSRSKWYNDYAVSVKSRITINPDTSKNQFSLELNSVTPEDTAVYYCARERKWELFLDYWGQGTLVTVSS

SEQ ID NO: 965 - protein, 119 aa, synthetic construct
EVQLVESGGGLVKPGGSLRLSCAASGFTFSSYSMNWVRQAPGKGLEWVSSISRSSSYIYYADSVKGRFTISRDNAKNSLYLQMNSLRAEDTAVYYCARERWELLYFDYWGQGTLVTVSS

SEQ ID NO: 966 - protein, 119 aa, synthetic construct
EVQLVESGGGLVKPGGSLRLSCAASGFTFSYYSMNWVRQAPGKGLEWVSSISRSTTYIYYADSVKGRFTISRDNAKNSLYLQMNSLRAEDTAVYYCARDGKGAIFLDYWGQGTLVTVSS

SEQ ID NO: 967 - protein, 117 aa, synthetic construct
EVQLVESGGGLVKPGGSLRLSCAASGFTFSSYTMNWVRQAPGKGLEWVSSITTSSSYIYYADSVKGRFTISRDNAKNSLYLQMNSLRAEDTAVYYCARVTFDAFDIWGQGTMVTVSS

SEQ ID NO: 968 - protein, 118 aa, synthetic construct
EVQLVESGGGLVKPGGSLRLSCAASGFTFSSYSMNWVRQAPGKGLEWVSSISRSSRYIFYADSVRGRFTISRDNAKNSLYLQMNSLRAEDTAVYYCAREKRELLFDYWGQGTLVTVSS

SEQ ID NO: 969 - protein, 118 aa, synthetic construct
EVQLVESGGGLVKPGGSLRLSCAASGFTFSIYSMNWVRQAPGKGLEWVSSISSGSSYIYYADSVKGRFTISRDNAKSSLFLQMNSLRAEDTAVYYCAREKRELLFDYWGQGTLVTVSS

SEQ ID NO: 970 - protein, 117 aa, synthetic construct
EVQLVESGGGLVKPGGSLRLSCVASGFTFSRYSMNWVRQAPGKGLEWVSSISSSSRYTYYADSLKGRFIMSRDNAKNSLFLQMNSLRAEDTAVYYCARNLYNALDYWGQGTLVTVSS

SEQ ID NO: 971 - protein, 118 aa, synthetic construct
EVQLVESGGGLVKPGGSLRLSCAASGFTFSSYTMNWVRQAPGKGLEWVSSISTSSRYIYYTDSVKGRFTISRDDAKNSLYLQMNSLRAEDTAVYYCTRNWGYDVFDIWGQGTMVIVSS

SEQ ID NO: 972 - protein, 118 aa, synthetic construct
EVQLVESGGGLVKPGGSLRLSCVVSGFTFSNYNMNWVRQAPGKGLEWVSSSSRSSYIYYADSVKGRFTISRDNAKNSLYLQMNSLRAEDTAIYYCAREKWELLFFDYWGQGTLVTVSS

SEQ ID NO: 973 - protein, 119 aa, synthetic construct
EVQLVESGGGLVKPGGSLRLSCTASGFTLSSYSMNWVRQAPGKGLEWVSSLSRSSNYIYYAVSVKGRFTISRDNAKNSLYLQMNSLRAEDTAVYYCARERWELLYFDYWGQGTLVTVSS

SEQ ID NO: 974 - protein, 118 aa, synthetic construct; identical to SEQ ID NO: 969

SEQ ID NO: 975 - protein, 119 aa, synthetic construct
EVQLVESGGGLVKPGGSLRLSCAASGFTFSSYSMNWVRQAPGKGLEWVSSISRSGTYIYYADSVKGRFTISRDNAKNSLYLQMNSLRAEDTAVYYCARERWELLYFDYWGQGTLVTVSS

SEQ ID NO: 976 - protein, 118 aa, synthetic construct
EVQLVESGGGLVKPGGSLRLSCAASGFTFSSYSMNWVRQAPGKGLEWVSSISRSSGYIFYADSVKGRFTISRDNAKNSLYLQMNSLRAEDTAVYYCVRERVGAGFDYWGQGTLVTVSS

SEQ ID NO: 977 - protein, 118 aa, synthetic construct
QVQLQESGPGLVKPSETLSLTCTVSGGSISSYYWSWIRQPAGKGLEWIGRIYTSGSTKYNPSLKSRVTMSVDTSKNQFSLKLISVTAADTAVYFCARERIRDLYFDYWGQGTLVTVSS

SEQ ID NO: 978 - protein, 119 aa, synthetic construct
EVQVAESGGGLVKPGGSLRLSCAASGFTFSRYSMNWVRQAPGKGLEWVSSISSSGSYTYYADSVKGRFTISRDNAKNSLYLQMNSLRAEDTAVYYCVRERGKGAGMDVWGQGTTVTVSS

SEQ ID NO: 979 - protein, 117 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAREYYYYGMDVWGQGTTVTVSS

SEQ ID NO: 980 - protein, 118 aa, synthetic construct
QVQLQESGPGLVKPSETLSLTCTVSGGSISSYYWSWIRQPAGKGLEWIGRIYASGSTYYNPSLKSRVTMSVDTSKNQFSLKLSSVTAADTAVYYCAREKRSRLYFDYWGQGTLVTVSS

SEQ ID NO: 981 - protein, 119 aa, synthetic construct
EVQLVESGGGLVKPGGSLRLSCAASGFTVSSYSMNWVRQAPGKGLEWVSSISRSSSYIYYADSVKGRFTISRDNAKNSLFLQMNSLRAEDTAVYYCARERWELLYFDYWGQGTLVTVSS

SEQ ID NO: 982 - protein, 119 aa, synthetic construct
EVQLVESGGGLVKPGGSLRLSCAASGFTFSSSIMNWVRQAPGKGLEWVSSISGSSSYIYYADSVKGRFTISRDNAKNSQYLQMKSLRAEDTAVYYCARERWNLLYFDYWGQGTLVTVSS

SEQ ID NO: 983 - protein, 117 aa, synthetic construct
EVQLVESGGGLVKPGGSLRLSCAASGFTFSTYTMNWVRQAPGKGLEWVSTITSSSRYIYYADSVKGRFTISRDNAKNSLYLQMNSLRAEDTAVYYCARVTFDAFDIWGQGTMVTVSS

SEQ ID NO: 984 - protein, 119 aa, synthetic construct
EVQLVESGGGLVKPGGSLRLSCAASGFTFSTYIMNWVRQAPGKGLEWVSSITSSSSYIYYADSVKGRFTISRDNAKNSLYLQMNSLRAEDTAVYYCAREKWELLFFDYWGQGTLVTVSS

SEQ ID NO: 985 - protein, 118 aa, synthetic construct
QVQLQESGPGLVKPSETLSLTCTVSGGSINSYYWSWIRQPAGKGLEWIGRIYTSGSTNYNPSLKSRVTMSVDTSKKQFSLKLSSVTAADTAVYYCARERISRLYFDYWGQGILVTVSS

SEQ ID NO: 986 - protein, 119 aa, synthetic construct
EVQLVESGGGLVKPGGSLRLSCAASGFTFSTYIMNWVRQAPGKGLEWVSSISSSRSYIYYADSVKGRFTISRDNAKNSLYLQMNSLRAEDTAVYYCAREDRYRLFFDYWGQGTLVTVSS

SEQ ID NO: 987 - protein, 119 aa, synthetic construct
EVQLVESGGGLVKPGGSLRLSCAASGFTITSYSMNWVRQAPGKGLEWVSTFSRSSTYIYYADSVKGRFTISRDNAKNSLYLQMNSLRAEDTAVYYCAREKWDLLYFDYWGQGTLVTVSS

SEQ ID NO: 988 - protein, 118 aa, synthetic construct
EVQLVESGGGLVKPGGSLRLSCAASGFTFSSYSMKWVRQAPGKGLEWVSSISSSSSYIYYADSVKGRFTISRDNAKNSLYLQMNSLRAEDTAVYYCARDRGELTLWYWGQGTLVTVSS

SEQ ID NO: 989 - protein, 118 aa, synthetic construct
EVQLVESGGGLVKPGGSLRLSCAASGFTFSTYSLNWVRQAPGKGLEWVSSISVSSRYIYFADSVKGRFTISRDNAKNSLYLQMNSLRAEDTAVYYCARDRRELLLDYWGQGTLVTVSS

SEQ ID NO: 990 - protein, 118 aa, synthetic construct
EVQLVESGGGLVKPGGSLRLSCAASGFTFSNYYMNWVRQAPGKGLERVSSISSSSNYIYYADSVKGRFTISRDNAKNSLYLQMNSLRAEDTAVYYCARDRRELPLDYWGQGTVVTVSS

SEQ ID NO: 991 - protein, 121 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDGELGIGTYFDYWGQGTLVTVSS

SEQ ID NO: 992 - protein, 119 aa, synthetic construct
EVQLVESGGGLVKPGGSLRLSCAASGFTVSNYSMNWVRQAPGKGLEWVSCISRSSSYIYYADSVKGRFTISRDNAKNSLFLQMNSLRAEDTAVYYCARERWELLYFDYWGQGTLVTVSS

SEQ ID NO: 993 - protein, 119 aa, synthetic construct; identical to SEQ ID NO: 992

SEQ ID NO: 994 - protein, 118 aa, synthetic construct
EVQLVESGGGLVKPGGSLRLSCAASGFTFSGYSMNWVRQAPGKGLEWVSSISSRSRYIYYADSVQGRFTISRDNSKNSLFLQMNSLRAEDTAVYYCGRDWRELLLDYWGQGTLVTVSS

SEQ ID NO: 995 - protein, 120 aa, synthetic construct
QVQLQESGPGLVKPSQTLSLTCTVSGGSISSGGYYWSWIRQHPGKGLEWIGYIYYSGSTYYNPSLKSRVTISVDTSKDQFSLKLSSVTAADTAVYYCARERKTSLPFDIWGQGTMVTVSS

SEQ ID NO: 996 - protein, 118 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRRELLLDYWGQGTLVTVSS

SEQ ID NO: 997 - protein, 117 aa, synthetic construct
EVQLVESGGGLVKPGGSLRLSCAASGFTFSSYSMNWVRQAPGKGLEWVSTISRSGGYIYDADSVKGRFTISRDNAKNSLYLQMNSLRAEDTAVYYCARDFNWTFDNWGQGTLVTVSS

SEQ ID NO: 998 - protein, 119 aa, synthetic construct
EVQLVESGGGLVKPGGSLRLSCAASGFTFSNFIMNWVRQAPGKGLEWVSSISGSSNYIYYADSVKGRFTISGDNAKNSLYLQMNSLRAEDTAVYYCAREKWVLLFFDYWGQGTLVTVSS

SEQ ID NO: 999 - protein, 117 aa, synthetic construct
EVQLVESGGGLVKPGGSLRLSCAASGFTFSSYSMNWVRQAPGKGLEWVSSISRRSGYIYYADSVKGRFTLSRDNAKKSLYLQMNSLRAEDTAVYYCARDLNWAFDYWGQGTLVTVSS

SEQ ID NO: 1000 - protein, 117 aa, synthetic construct
EVQLVESGGGLVKPGGSLRLSCAASGFTFSRYSMNWVRQAPGKGLEWVSTISRSSRYIYYGDSVKGRFTISRDNAKNSLLLQMNSLRAEDTAVYYCARDHNWSFDYWGKGILVTVSS

SEQ ID NO: 1001 - protein, 119 aa, synthetic construct
EVQLVESGGGLVKFGGSLRLSCAASGFTFSRYSMNWVRQAPGKGLEWVSSISSSSSYKYYADSVKGRFTISRDNAKNSLYLQMNSLRAEDTAVYYCAREGGNSHGMDVWGQGTTVTVSS

SEQ ID NO: 1002 - protein, 118 aa, synthetic construct
EVQLVESGGGLVQPGGSLRLSCAASGFTFSRYWMSWVRQAPGKGLEWVANIKEDGSEKYYVDSVKGRFTISRDNAKNSLYLQMNSLRAEDTAVYFCARERRTGYFDYWGQGTLVTVSS

SEQ ID NO: 1003 - protein, 119 aa, synthetic construct
EVQLVESGGGLVQPGGSLRLSCAASGFTFSSYWMSWVRQAPGKGLEWVSSISSRRGYKYHADSVKGRFTISRDNAKNSLYLQMNSLRAEDTAVYYCARVPYSGSYFDYWGQGTLVTVSS

SEQ ID NO: 1004 - protein, 117 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCTNYRYNWNDYWGQGTLVTVSS

SEQ ID NO: 1005 - protein, 117 aa, synthetic construct
EVQLVESGGGLVKPGGSLRLSCAASGFTFSYYSMNWVRQAPGKGLEWVSSISSSSTYIYYADSVKGRFTISRDNAKNSLYLQMNSLRAEDTAVYYCANYRYNWNDYWGQGTLVTVSS

SEQ ID NO: 1006 - protein, 121 aa, synthetic construct
EVQLVESGGGLVKPGGSLRLSCAASGFTFSSYSMNWVRQAPGKGLEWVSYISSSSRYIYYADSVKGRFTISRDNAKNSLYLQMNSLRAEDTAVYYCAREGPNWGTHAFDIWGQGTMVTVSS

SEQ ID NO: 1007 - protein, 117 aa, synthetic construct
EVQLVESGGALVKPGGSLRLSCAASGFTFSRYSMNWVRQAPGKGLEWVSSISSSSSYIYYADSVKGRFTISRDNAKNSLYLQMNSLRAEDTAVYYCANYRYNWNDYWGQGTLVAVSS

SEQ ID NO: 1008 - protein, 117 aa, synthetic construct
EVQLVESGGGLVKPGGSLRLSCSASGFTFSSYSMNWVRQAPGKGLEWVSSISRSSSYIFYADSVKGRFTISRDNAKNSLYLQMNSLRAEDTAVYYCARERRNGFDYWGQGTLVTVSS

SEQ ID NO: 1009 - protein, 117 aa, synthetic construct
EVQLVESGGGLVKPGGSLRLSCAASGFTFSRYSMNWVRQAPGKGLEWVSSISRSSSYIYYADSVKGRFTISRDNAKNSLYLQMNSLRAEDTAVYYCANYRYNWNDYWGQGTLVTVSS

SEQ ID NO: 1010 - protein, 117 aa, synthetic construct
EVQLVESGGGLVKPGGSLRLSCAASGFTFSRYSMNWVRQAPGKGLEWVSSISSSSSYIYYADSVKGRFTISRDNAKNSLYLQMNSLRAEDTAVYYCARAVYNYFDQWGQGTLVTVSS

SEQ ID NO: 1011 - protein, 121 aa, synthetic construct
EVQLVESGGGLVKPGGSLRLSCAASGFTFSSYSMNWVRQAPGKGLEWVSYISRSSTYIYYADSMKGRFTISRDNAKNSLYLQMNSLRAEDTAVYYCAREGPNWGTHAFDIWGQGTMVTVSS

SEQ ID NO: 1012 - protein, 118 aa, synthetic construct
EVQLVESGGGLVKPGGSLRLSCAASGLTFSSYSMNWVRQAPGKGLEWVSSISSSSSYIYYADSVKGRFTISRDNAKNSLYLQMNSLRAEDTAVYYCARERRELAFDIWGQGTMVTVSS

SEQ ID NO: 1013 - protein, 118 aa, synthetic construct
EVQLVESGGGLVKPGGSLRLSCAASGITFSSYSMNWVRQAPGKGLEWVSSISSSSSYIYYADSVKGRFTISRDNAKNSLHLQMNSLRAEDTAVYYCARERRELSFDYWGQGTLVTVSS

SEQ ID NO: 1014 - protein, 121 aa, synthetic construct
EVQLVESGRGLVNPGGSLRLSCAASGFTFSSYSMNWVRQAPGKGLEWVSYISSSSNYIYYADSVKGRFTISRDNAKNSLYLQMNSLRAEDTAVYYCAREGPNWGTHAFDIWGQGTMVTVSS

SEQ ID NO: 1015 - protein, 117 aa, synthetic construct
EVQLVESGGGLVNPGGSLRLSCAASGFTFSRYSMNWVRQAPGKGLEWVSSISSSSSYIYYADSVKGRFTISRDNAKNSLYLQMNSLRAEDTAVYYCANYRYNWNDYWGQGTLVTVSS

SEQ ID NO: 1016 - protein, 118 aa, synthetic construct
EVQLVESGGGLVKPGGSLRLSCAASGITFSSYSMNWVRQAPGKGLEWVSSISSSSSYIYYADSVKGRFTISRDNAKNSLHLQMHSLRAEDTAVYYCARERRELSFDYWGQGTLVTVSS

SEQ ID NO: 1017 - protein, 117 aa, synthetic construct
EVQLVESGGGLVKPGGSLRLSCAASGFTFSRYSMNWVRQAPGKGLEWVSSISSSSSYISYADSVKGRFTISRDNAKNSLFLQMNSLRAEDTAVYYCARVSYNSFDYWGQGTLVTVSS

SEQ ID NO: 1018 - protein, 117 aa, synthetic construct; identical to SEQ ID NO: 1010

SEQ ID NO: 1019 - protein, 117 aa, synthetic construct; identical to SEQ ID NO: 1015

SEQ ID NO: 1020 - protein, 117 aa, synthetic construct
EVQLVESGGGLVKPGGSLRLSCAASGFTFSSYSMNWVRQAPGKGLEWVSSISRSSSYIFYADSVKGRFTISRDNAKNSLYLQMNSLRAEDTAVYYCARERRNGFDYWGQGTLVTVSS

SEQ ID NO: 1021 - protein, 118 aa, synthetic construct
EVQLVQSGAEVKKPGESLRISCKGSGYSFTTYWIAWVRQMPGKGLEWMGFIYPGDSDTIYSPSFQGQVTISADKSISTAYLQWSSLRASDTAMYYCARQGYLDAFDIWGQGTMVTVSS

SEQ ID NO: 1022 - protein, 117 aa, synthetic construct
EVQLVESGGGLVKPGGSLRLSCAASGFTFSRYSMNWVRQAPGKGLEWVSSISSSSRYIYYADSVKGRFTISRDNANNSLYLHMNSLRAEDTAVYYCARALYNYFDYWGQGTLVTVSS

SEQ ID NO: 1023 - protein, 117 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCANYRYNWNDYWGQGTLVTVSS

SEQ ID NO: 1024 - protein, 118 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARERRDLAFDIWGQGTMVTVSS

SEQ ID NO: 1025 - protein, 121 aa, synthetic construct; identical to SEQ ID NO: 1011

SEQ ID NO: 1026 - protein, 118 aa, synthetic construct; identical to SEQ ID NO: 1013

SEQ ID NO: 1027 - protein, 118 aa, synthetic construct; identical to SEQ ID NO: 1016

SEQ ID NO: 1028 - protein, 119 aa, synthetic construct
EVQLVESGGGLVKPGGSLRLSCAASGFTLSSYSMNWVRQAPGKCLEWVSSISRSGSYIYYADSVKGRFTISRDNAKNSLYLQMNSLRAEDTAVYYCAREKWELLYFDYWGQGTLVTVSS

SEQ ID NO: 1029 - protein, 117 aa, synthetic construct
EVQLVESGGGLVKPGGSLRLSCAASGFTFSSYSMNWVRQAPGKCLEWVSSISRSTSYIYYADSVKGRFTISRDNAKNSLYLQMNSLRAEDTAVYYCARGSRDAFDIWGQGTMVTVSS

SEQ ID NO: 1030 - protein, 119 aa, synthetic construct
EVQLVESGGGLVKPGGSLRLSCAASGFTFSSYSMNWVRQAPGKCLEWVSYISRSSNYIHYADSVKGRFTIFRDNAKNSLYLQMNSLRVEDTAVYYCARAERGSYAVDIWGQGTMVTVSS

SEQ ID NO: 1031 - protein, 118 aa, synthetic construct
VQLVESGGGLVKPGGSLRLSCAASGFTFSSYNMNWVRQAPGKCLEWVSSISSSSSYIYYADSVKGRFTISRDNAKNSLYLQMNSLRAEDTAVFYCARAERGSYAFDIWGQGTMVTVSS

SEQ ID NO: 1032 - protein, 117 aa, synthetic construct
EVQLVESGGGLVQPGGSLRLSCAASGFTFSRYSMNWVRQAPGKCLEWVSYISSSSSSIYYADSVKGRFTISRDNAKNSLYLQMNSLRDEDTAVYYCARDRRATLDYWGQGTLVTVSS

SEQ ID NO: 1033 - protein, 118 aa, synthetic construct
EVQLVDSGGGLVKPGGSLRLSCAASGFTFSSYSMNWVRQAPGKCLEWVSSISSRGSYRNYADSVKGRFTISRDNAKNSLYLQMNSLRAEDTAVYYCARDRRELLLDCWGQGTLVTVSS

SEQ ID NO: 1034 - protein, 118 aa, synthetic construct
EVQLVESGGGLVKPGGSLRLSCAASGFTFSRYSMNWVRQAPGKCLKWVSSISSSGSYKYYADSVKGRFTISRDNAKNSLYLQMNSLRAEDTAVYYCAREPPTLGMDYWGQGTLVTVSS

SEQ ID NO: 1035 - protein, 118 aa, synthetic construct
EVQLVESGGGLVKPGGSLRLSCAASGFTFSSYSMNWVRQAPGKCLEWVSSISSSSSYIYYADSVKGRFTISRANAKNSLYLQMSSLRAEDTAVYYCARDRRELLLDYWGQGTLVTVSS

SEQ ID NO: 1036 - protein, 118 aa, synthetic construct
EVQLVDSGGGLVKPGGSLRLSCAASGFTFSSYSMNWVRQAPGKCLEWVSSVNSRGSYIYYADSVKGRFTISRDNAKNSLYLQMNSLRADDTAVYYCARDRRELLLDYWGQGTLVTVSS

SEQ ID NO: 1037 - protein, 122 aa, synthetic construct
QVQLQQSGPGLVKPSQTLSLTCAISGDSVSCNSVAWNWIRQSPSRCLEWLGRTYYRSKWHNEYAVSVKSRINIKPDTSKNQVSLQLKSVTPEDTAVYYCARERKWELFLDYWGQGTLVTVSS

SEQ ID NO: 1038 - protein, 121 aa, synthetic construct
QVTLKESGPGILQPSQTLSLTCSFSGFSLSTFGMGVGWIRQPSGKCLEWLAHIWWDDDKYYNPALKSRLTISKDTSKNQVFLKIANVDTADTATYYCARLGYYPYWFFDVWGTGTTVTVSS

SEQ ID NO: 1039 - protein, 118 aa, synthetic construct
EVQLVESGGGLVKPGGSLRLSCAASGFTFSSYTMNWVRQAPGKCLEWVSSISRSSSYKYYADSVKGRFTISRDNAKNSLYLQMNSLRAEDTAVYYCAREPPTLGMDYWGQGTLVIVSS

SEQ ID NO: 1040 - protein, 118 aa, synthetic construct
EVQLVESGGGLVQPGGSLRLSCAASGFTFSNYWMSWVRQAPGKCLEWVANIKEDGSEKYYVDSVKGRFTISRDNAKNSLYLQMNSLTAEDTSVYYCARERRTGYFDYWGQGTLVTVSS

SEQ ID NO: 1041 - protein, 119 aa, synthetic construct
EVQLVESGGGLVKPGGSLRLSCAASGFTCSSYSMTWVRQAPGKCLEWVSSISRRSSYIYYADSVKGRFTISRDNAKNSLYLQLNSLRAEDTAVYYCARERWELLYFDYWGQGTLVTVSS

SEQ ID NO: 1042 - protein, 119 aa, synthetic construct
EVQLVESGGGLVKPGGSLRLSCAASGFNCSSYSMNWVRQAPGKCLEWVSSISRSSNYIYYADSVKGRFSISRDDAKNSLYLQMNSLRAEDTAVYYCARERWIQLYSDYWGQGILVTVSS

SEQ ID NO: 1043 - protein, 118 aa, synthetic construct
EVQLVESGGGLVQPGGSLRLSCAASGFTFSRYWMTWVRQAPGKCLEWVANIKEDGSEKYCVDSVKGRFTISRDNAKNSLYLQLNSLRAEDTAVYYCARERRTGYFDYWGQGTLVTVSS

SEQ ID NO: 1044 - protein, 119 aa, synthetic construct
EVQLVESGGGLVQPGGSLRLSCAASGFTFSRYSINWVRQAPGKCLEWVSYISSSSSTIYYADSVKGRFTISRDNAKNSLYLQMNSLRDEDTAVYYCARERWELLYFDYWGQGTLVTVSS

SEQ ID NO: 1045 - protein, 115 aa, synthetic construct
EVQLVESGEGLVQPGGSLRLSCAASGFTFSSYAMHWVRQAPGKCLEYVSAISSNGGRTYYADSVKGRFTISRDNSKNTLYLQMGSLRAEDMAVYYCARGGSLDYWGQGTLVTVSS

SEQ ID NO: 1046 - protein, 119 aa, synthetic construct
EVQLVESGGGLVKPGGSLRLSCAASGFTLSSYSMNWVRQAPGKCLEWVSSISRSSSYIYYADSVKGRFTISRDNAKNSLYLQMNSLRAEDTAVYYCARERWELLYFDYWGQGTLVTVSS

SEQ ID NO: 1047 - protein, 118 aa, synthetic construct
EVQLVESGGGLVKPGGSLRLSCAASGLTFSTYSMNWVRQAPGKCLEWVSSISSSGSYVYYADSVKGRFTMSRDNAKNSLYLQMNSLRAEDTAVYYCARERRELLFDYWGQGTLVTVSS

SEQ ID NO: 1048 - protein, 119 aa, synthetic construct
EVQLVESGGGLVKPGGSLRLSCAASGFTFSSYSMNWVRQAPGKCLEWVSSISRSSSYIYYADSVKGRFTLSRDNAKNSLYLQMNSLRGEDTAIYYCGRDGKGAIFLDYWGQGTLVTVSS

SEQ ID NO: 1049 - protein, 119 aa, synthetic construct
EVQLVESGGGLVQPGGSLRLSCAASGFTFSSYSMNWVRQAPGKCLEWVSYISSSSSTIYYADSVKGRFTISRDNAKNSLYLQMNSLRDEDTAVYYCARDGPVDYGMDVWGQGTTVTVSS

SEQ ID NO: 1050 - protein, 118 aa, synthetic construct
EVHLVESGGGLVKPGGSLRLSCAASGFTFSSYSMNWVRQAPGKCLEWVSSISSSSTYIYYADSVKGRFTISRDNAKNSLYLQMNSLRAEDTAVYYCAREPRTLGMDYWGQGILVTVSS

SEQ ID NO: 1051 - protein, 118 aa, synthetic construct
EVQLVESGGGLVKPGGSLRLSCVASGFTFSSYTMNWVRQAPGKCLEWVSSISRSGSYKYNADSVKGRFTISRDNAKNSLYLQMNSLRVEDTAVYYCAREPPTLGMDYWGQGTLVTVSS

SEQ ID NO: 1052 - protein, 118 aa, synthetic construct; identical to SEQ ID NO: 1050

SEQ ID NO: 1053 - protein, 118 aa, synthetic construct; identical to SEQ ID NO: 1050

SEQ ID NO: 1054 - protein, 118 aa, synthetic construct
QVQLVQSGAEVKKPGASLKVSCKASGYTFTSYYMHWVRQAPGQCLEWMGIINPSNGNTISAQKFQGRVTMTRDTSTSTVYMEMSSLRSEDTAVYYCVRSWGYYVMDVWGQGTTVTVSS

SEQ ID NO: 1055 - protein, 122 aa, synthetic construct
QVQLQQSGPGLVKPSQTLSLTCAISGDSVSSNSVAWNWIRQSPSRCLEWLGRTYYRSKWYNECAGSVKSRITINPDTSKNQFSLQLNSVTPEDTAIYYCARERKWELFLDYWGQGTLVTVSS

SEQ ID NO: 1056 - protein, 118 aa, synthetic construct
EVQLVQSGAEVKKPGESLKISCKGSGYSFISYWIAWVRQMPGKCLEWMGIIYPGDSETRYRPSFQGQVTISADKSINTAYLQWSSLKAADTAMYYCARQGAYDGFDIWGQGTMVTVSS

SEQ ID NO: 1057 - protein, 117 aa, synthetic construct
QVQLQESGPGLVKPSETLSLTCTVSGGSINSYYWSWIRQSPGKCLEWIGHIYYSGRTKYNPSLKSRVTISVDTSKNQFSLKLSSVTAADTAVYYSARENRNGAFDIWGQGTVVTVSS

SEQ ID NO: 1058 - protein, 121 aa, synthetic construct
EVQLVQSGAEVKKPGESLKISCKGSGYSFTNYWIGWVRQMPGKCLEWMGIIYPADSDTTYSPSFQGQVTISADKSITTAYLQWSSLKASDTAMYYCARHGGSYILDAFDIWGQGTMVTVSS

SEQ ID NO: 1059 - protein, 119 aa, synthetic construct
EVQLVESGGGLVKPGGSLRLSCEASGFTFSSYNMNWVRQAPGKCLEWVSSISSSSSYIYYTDSVKGRFTISRDNAKNSLYLQMNSLRAEDTAVYYCARAERGSYAFDFWGQGTMVTVSS

SEQ ID NO: 1060 - protein, 118 aa, synthetic construct
EVQLVESGGGLVKPGGSLRLSCAASGFTFSSYSMNWVRQAPGKCLEWVSSISRSSSYIYYADSVKGRFTISRDNAKNSLYLQMNSLRAEDTAVYYCAREPPTLGMDYWGQGTLVTVSS

SEQ ID NO: 1061 - protein, 118 aa, synthetic construct
EVHLVESGGGLVKPGGSLRLSCAASGFTFSRYSMNWVRQAPGKCLEWVSSISRSSSYIYYADSVKGRFTISRDNAKNSLYLQMNSLRAEDTAVYYCAREPRTLGMDYWGQGILVTVSS

SEQ ID NO: 1062 - protein, 118 aa, synthetic construct
EVQLVESGGGLVKPGGSLRLSCAASGFTFSSYSMNWVRQAPGKCLEWVSSVNSRGSYIYYADSVKGRFTISRDNAKDSLYLQMNSLRADDTAVYYCARDRRELLLDYWGQGTLVTVSS

SEQ ID NO: 1063 - protein, 118 aa, synthetic construct
EVQLVESGGGLVQRGGSLRLSCAASGFTFSNYWMSWVRQAPGKCLEWVANIKEDGSEKYYVDSVKGRFTISRDNAKHSLYLQMNSLRAEDTAVYYCARERRTGYFDYWGQGTLVTVSS

SEQ ID NO: 1064 - protein, 119 aa, synthetic construct
EVQLVESGGGLVQPGGSLRLSCAASGFSFSSYSMNWVRQAPGKCLEWVSHIRSSTYSIYYADSVMGRFTISRDNAKNSLYLQMNSLRDEDTAVYFCARAKTGGDAFDIWGQGTMVTVSS

SEQ ID NO: 1065 - protein, 118 aa, synthetic construct
QGQLQQWGAGLLKPSETLSLTCAVYGGSFSGYYWSWIRLPPGKCLEWIGEINHSGNTNYNPSLKSRVTISVDTSKNQFSLQLSSVTAADTAVYYCAKEVEIWGYFDYWGQGTLVTVSS

SEQ ID NO: 1066 - protein, 122 aa, synthetic construct
EVQLLESGGGLVQPGGSLRLSCAASGFTFSYYAMSWVRQAPGKCLEWVSAISGSGGSTYYADSVKGRFTISRDNSKNTLYLQMNSLRAEDTAVYYCAKEGIVGARAEYFQHWGQGTLVTVSS

SEQ ID NO: 1067 - protein, 122 aa, synthetic construct; identical to SEQ ID NO: 1066

SEQ ID NO: 1068 - protein, 118 aa, synthetic construct; identical to SEQ ID NO: 1062

SEQ ID NO: 1069 - protein, 119 aa, synthetic construct
EVQLVESGGGLVQPGGSLRLSCAASGFTFKSYSINWVRQAPGKCLEWVSYISSSSSTIYYADSVKGRFTISRDNAKNSLYLQMNSLRDEDTAVYYCARDGPVDYGMDVWGQGTTVTVSS

SEQ ID NO: 1070 - protein, 118 aa, synthetic construct
EVQLVQSGAEVKKPGESLKISCKGSEYSFTTYWIAWVRQMPGKCLEWMGIIYPGDSDTTYSPSFQGQVTISADKSISIAFLQWSSLKASDTAMYYCARQGELDAFDIWGQGTMVTVSS

SEQ ID NO: 1071 - protein, 114 aa, synthetic construct
EVQLVESGGGLVQPGGSLRLSCAASGFTFSTYWMSWVRQAPGKCLEWVANIKQDGSEKYYVDSVKGRFTISRDNAKNSLYLQMNSLRAEDTAVYYCATGSFDYWGQGTLVTVSS

SEQ ID NO: 1072 - protein, 118 aa, synthetic construct
EVQLVESGGGLVQPGGSLRLSCAASGFTFSSYWMSWVRQAPGKCLEWVANIKEDGSEKYYVDSVKGRFTISRDNAKNSLYLQMNSLRAEDTAVYYCARERRTGYLDYWGQGTLVTVSS

SEQ ID NO: 1073 - protein, 118 aa, synthetic construct
EVQLVESGGGLVKPGGSLRLSCSASGFTFSTYTMNWVRQAPGKCLDWVSSISKSSTYIYYADSVKGRFTISRDNAKNSLYLQMNSLRAEDTAVYYCARERRTLAFDIWGQGTMVTVSS

SEQ ID NO: 1074 - protein, 119 aa, synthetic construct
EVQLVESGGGLVQPGGSLRLSCAASGFTFSRYSMNWVRQAPGKCLEWVSYISRSSTTIYYADSVKGRFTISRDNAKNSLYLQMNSLRDEDAAVYYCARERWELLYFDYWGQGTLVTVSS

SEQ ID NO: 1075 - protein, 118 aa, synthetic construct
EVQLVESGGGLVKPGGSLRLSCAASGFTFSSYTMNWVRQAPGKCLEWVSSISGSGSYIYYADSVKGRFTISRDNAKNSLYLQMNSLRAEDTAVYYCAREPPTLGMDYWGQGTLVTVSS

SEQ ID NO: 1076 - protein, 118 aa, synthetic construct
EVQLVESGGGLVKPGGSLRLSCSASGFTFSTYTMNWVRQAPGKCLEWVSSISKSSSYIYYTDSVKGRFTISRDNAKNSLYLQMNSLRAEDTAVYYCARERRTLAFDIWGQGTMVTVSS

SEQ ID NO: 1077 - protein, 122 aa, synthetic construct
QVQLQQSGPGLVKPSQTLSLTCAISGDSVSSNSVAWNWIRQSPSRCLEWLGRTYSRSKWYNDYAVSVKSRITINPDTSKNQFSLELNSVTPEDTAVYYCARERKWELFLDYWGQGTLVTVSS

SEQ ID NO: 1078 - protein, 119 aa, synthetic construct
EVQLVESGGGLVKPGGSLRLSCAASGFTFSSYSMNWVRQAPGKCLEWVSSISRSSSYIYYADSVKGRFTISRDNAKNSLYLQMNSLRAEDTAVYYCARERWELLYFDYWGQGTLVTVSS

SEQ ID NO: 1079 - protein, 119 aa, synthetic construct
EVQLVESGGGLVKPGGSLRLSCAASGFTFSYYSMNWVRQAPGKCLEWVSSISRSTTYIYYADSVKGRFTISRDNAKNSLYLQMNSLRAEDTAVYYCARDGKGAIFLDYWGQGTLVTVSS

SEQ ID NO: 1080 - protein, 117 aa, synthetic construct
EVQLVESGGGLVKPGGSLRLSCAASGFTFSSYTMNWVRQAPGKCLEWVSSITTSSSYIYYADSVKGRFTISRDNAKNSLYLQMNSLRAEDTAVYYCARVTFDAFDIWGQGTMVTVSS

SEQ ID NO: 1081 - protein, 118 aa, synthetic construct
EVQLVESGGGLVKPGGSLRLSCAASGFTFSSYSMNWVRQAPGKCLEWVSSISRSSRYIFYADSVRGRFTISRDNAKNSLYLQMNSLRAEDTAVYYCAREKRELLFDYWGQGTLVTVSS

SEQ ID NO: 1082 - protein, 118 aa, synthetic construct
EVQLVESGGGLVKPGGSLRLSCAASGFTFSIYSMNWVRQAPGKCLEWVSSISSGSSYIYYADSVKGRFTISRDNAKSSLFLQMNSLRAEDTAVYYCAREKRELLFDYWGQGTLVTVSS

SEQ ID NO: 1083 - protein, 117 aa, synthetic construct
EVQLVESGGGLVKPGGSLRLSCVASGFTFSRYSMNWVRQAPGKCLEWVSSISSSSRYTYYADSLKGRFIMSRDNAKNSLFLQMNSLRAEDTAVYYCARNLYNALDYWGQGTLVTVSS

SEQ ID NO: 1084 - protein, 118 aa, synthetic construct
EVQLVESGGGLVKPGGSLRLSCAASGFTFSSYTMNWVRQAPGKCLEWVSSISTSSRYIYYTDSVKGRFTISRDDAKNSLYLQMNSLRAEDTAVYYCTRNWGYDVFDIWGQGTMVIVSS

SEQ ID NO: 1085 - protein, 118 aa, synthetic construct
EVQLVESGGGLVKPGGSLRLSCVVSGFTFSNYNMNWVRQAPGKCLEWVSSSSRSSYIYYADSVKGRFTISRDNAKNSLYLQMNSLRAEDTAIYYCAREKWELLFFDYWGQGTLVTVSS

SEQ ID NO: 1086 - protein, 119 aa, synthetic construct
EVQLVESGGGLVKPGGSLRLSCTASGFTLSSYSMNWVRQAPGKCLEWVSSLSRSSNYIYYAVSVKGRFTISRDNAKNSLYLQMNSLRAEDTAVYYCARERWELLYFDYWGQGTLVTVSS

SEQ ID NO: 1087 - protein, 118 aa, synthetic construct; identical to SEQ ID NO: 1082

SEQ ID NO: 1088 - protein, 119 aa, synthetic construct
EVQLVESGGGLVKPGGSLRLSCAASGFTFSSYSMNWVRQAPGKCLEWVSSISRSGTYIYYADSVKGRFTISRDNAKNSLYLQMNSLRAEDTAVYYCARERWELLYFDYWGQGTLVTVSS

SEQ ID NO: 1089 - protein, 118 aa, synthetic construct
EVQLVESGGGLVKPGGSLRLSCAASGFTFSSYSMNWVRQAPGKCLEWVSSISRSSGYIFYADSVKGRFTISRDNAKNSLYLQMNSLRAEDTAVYYCVRERVGAGFDYWGQGTLVTVSS

SEQ ID NO: 1090 - protein, 118 aa, synthetic construct
QVQLQESGPGLVKPSETLSLTCTVSGGSISSYYWSWIRQPAGKCLEWIGRIYTSGSTKYNPSLKSRVTMSVDTSKNQFSLKLISVTAADTAVYFCARERIRDLYFDYWGQGTLVTVSS

SEQ ID NO: 1091 - protein, 119 aa, synthetic construct
EVQVAESGGGLVKPGGSLRLSCAASGFTFSRYSMNWVRQAPGKCLEWVSSISSSGSYTYYADSVKGRFTISRDNAKNSLYLQMNSLRAEDTAVYYCVRERGKGAGMDVWGQGTTVTVSS

SEQ ID NO: 1092 - protein, 117 aa, synthetic construct
QVQLQQWGAGLLKPSETLSLTCAVYGGSFSGYYWSWIRQPPGKCLEWIGEIQHSGSTNYNPSLKSRVTISVDTSKNQFSLKLSSVTAADTAVYYCAREYYYYGMDVWGQGTTVTVSS

SEQ ID NO: 1093 - protein, 118 aa, synthetic construct
QVQLQESGPGLVKPSETLSLTCTVSGGSISSYYWSWIRQPAGKCLEWIGRIYASGSTYYNPSLKSRVTMSVDTSKNQFSLKLSSVTAADTAVYYCAREKRSRLYFDYWGQGTLVTVSS

SEQ ID NO: 1094 - protein, 119 aa, synthetic construct
EVQLVESGGGLVKPGGSLRLSCAASGFTVSSYSMNWVRQAPGKCLEWVSSISRSSSYIYYADSVKGRFTISRDNAKNSLFLQMNSLRAEDTAVYYCARERWELLYFDYWGQGTLVTVSS

SEQ ID NO: 1095 - protein, 119 aa, synthetic construct
EVQLVESGGGLVKPGGSLRLSCAASGFTFSSSIMNWVRQAPGKCLEWVSSISGSSSYIYYADSVKGRFTISRDNAKNSQYLQMKSLRAEDTAVYYCARERWNLLYFDYWGQGTLVTVSS

SEQ ID NO: 1096 - protein, 117 aa, synthetic construct
EVQLVESGGGLVKPGGSLRLSCAASGFTFSTYTMNWVRQAPGKCLEWVSTITSSSRYIYYADSVKGRFTISRDNAKNSLYLQMNSLRAEDTAVYYCARVTFDAFDIWGQGTMVTVSS

SEQ ID NO: 1097 - protein, 119 aa, synthetic construct
EVQLVESGGGLVKPGGSLRLSCAASGFTFSTYIMNWVRQAPGKCLEWVSSITSSSSYIYYADSVKGRFTISRDNAKNSLYLQMNSLRAEDTAVYYCAREKWELLFFDYWGQGTLVTVSS

SEQ ID NO: 1098 - protein, 118 aa, synthetic construct
QVQLQESGPGLVKPSETLSLTCTVSGGSINSYYWSWIRQPAGKCLEWIGRIYTSGSTNYNPSLKSRVTMSVDTSKKQFSLKLSSVTAADTAVYYCARERISRLYFDYWGQGILVTVSS

SEQ ID NO: 1099 - protein, 119 aa, synthetic construct
EVQLVESGGGLVKPGGSLRLSCAASGFTFSTYIMNWVRQAPGKCLEWVSSISSSRSYIYYADSVKGRFTISRDNAKNSLYLQMNSLRAEDTAVYYCAREDRYRLFFDYWGQGTLVTVSS

SEQ ID NO: 1100 - protein, 119 aa, synthetic construct
EVQLVESGGGLVKPGGSLRLSCAASGFTITSYSMNWVRQAPGKCLEWVSTFSRSSTYIYYADSVKGRFTISRDNAKNSLYLQMNSLRAEDTAVYYCAREKWDLLYFDYWGQGTLVTVSS

SEQ ID NO: 1101 - protein, 118 aa, synthetic construct
EVQLVESGGGLVKPGGSLRLSCAASGFTFSSYSMKWVRQAPGKCLEWVSSISSSSSYIYYADSVKGRFTISRDNAKNSLYLQMNSLRAEDTAVYYCARDRGELTLWYWGQGTLVTVSS

SEQ ID NO: 1102 - protein, 118 aa, synthetic construct
EVQLVESGGGLVKPGGSLRLSCAASGFTFSTYSLNWVRQAPGKCLEWVSSISVSSRYIYFADSVKGRFTISRDNAKNSLYLQMNSLRAEDTAVYYCARDRRELLLDYWGQGTLVTVSS

SEQ ID NO: 1103 - protein, 118 aa, synthetic construct
EVQLVESGGGLVKPGGSLRLSCAASGFTFSNYYMNWVRQAPGKCLERVSSISSSSNYIYYADSVKGRFTISRDNAKNSLYLQMNSLRAEDTAVYYCARDRRELPLDYWGQGTVVTVSS

SEQ ID NO: 1104 - protein, 121 aa, synthetic construct
EVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARDGELGIGTYFDYWGQGTLVTVSS

SEQ ID NO: 1105 - protein, 119 aa, synthetic construct
EVQLVESGGGLVKPGGSLRLSCAASGFTVSNYSMNWVRQAPGKCLEWVSCISRSSSYIYYADSVKGRFTISRDNAKNSLFLQMNSLRAEDTAVYYCARERWELLYFDYWGQGTLVTVSS

SEQ ID NO: 1106 - protein, 119 aa, synthetic construct
EVQLVESGGGLVKPGGSLRLSCAASGFTVSNYSMNWVRQAPGKCLEWVSSISRSSSYIYYADSVKGRFTISRDNAKNSLFLQMNSLRAEDTAVYYCARERWELLYFDYWGQGTLVTVSS

SEQ ID NO: 1107 - protein, 118 aa, synthetic construct
EVQLVESGGGLVKPGGSLRLSCAASGFTFSGYSMNWVRQAPGKCLEWVSSISSRSRYIYYADSVQGRFTISRDNSKNSLFLQMNSLRAEDTAVYYCGRDWRELLLDYWGQGTLVTVSS

SEQ ID NO: 1108 - protein, 120 aa, synthetic construct
QVQLQESGPGLVKPSQTLSLTCTVSGGSISSGGYYWSWIRQHPGKCLEWIGYIYYSGSTYYNPSLKSRVTISVDTSKDQFSLKLSSVTAADTAVYYCARERKTSLPFDIWGQGTMVTVSS

SEQ ID NO: 1109 - protein, 118 aa, synthetic construct
EVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARDRRELLLDYWGQGTLVTVSS

SEQ ID NO: 1110 - protein, 117 aa, synthetic construct
EVQLVESGGGLVKPGGSLRLSCAASGFTFSSYSMNWVRQAPGKCLEWVSTISRSGGYIYDADSVKGRFTISRDNAKNSLYLQMNSLRAEDTAVYYCARDFNWTFDNWGQGTLVTVSS

SEQ ID NO: 1111 - protein, 119 aa, synthetic construct
EVQLVESGGGLVKPGGSLRLSCAASGFTFSNFIMNWVRQAPGKCLEWVSSISGSSNYIYYADSVKGRFTISGDNAKNSLYLQMNSLRAEDTAVYYCAREKWVLLFFDYWGQGTLVTVSS

SEQ ID NO: 1112 - protein, 117 aa, synthetic construct
EVQLVESGGGLVKPGGSLRLSCAASGFTFSSYSMNWVRQAPGKCLEWVSSISRRSGYIYYADSVKGRFTLSRDNAKKSLYLQMNSLRAEDTAVYYCARDLNWAFDYWGQGTLVTVSS

SEQ ID NO: 1113 - protein, 117 aa, synthetic construct; identical to SEQ ID NO: 1112

SEQ ID NO: 1114 - protein, 117 aa, synthetic construct
EVQLVESGGGLVKPGGSLRLSCAASGFTFSRYSMNWVRQAPGKCLEWVSTISRSSRYIYYGDSVKGRFTISRDNAKNSLLLQMNSLRAEDTAVYYCARDHNWSFDYWGKGILVTVSS

SEQ ID NO: 1115 - protein, 119 aa, synthetic construct
EVQLVESGGGLVKFGGSLRLSCAASGFTFSRYSMNWVRQAPGKCLEWVSSISSSSSYKYYADSVKGRFTISRDNAKNSLYLQMNSLRAEDTAVYYCAREGGNSHGMDVWGQGTTVTVSS

SEQ ID NO: 1116 - protein, 118 aa, synthetic construct
EVQLVESGGGLVQPGGSLRLSCAASGFTFSRYWMSWVRQAPGKCLEWVANIKEDGSEKYYVDSVKGRFTISRDNAKNSLYLQMNSLRAEDTAVYFCARERRTGYFDYWGQGTLVTVSS

SEQ ID NO: 1117 - protein, 119 aa, synthetic construct
EVQLVESGGGLVQPGGSLRLSCAASGFTFSSYWMSWVRQAPGKCLEWVSSISSRRGYKYHADSVKGRFTISRDNAKNSLYLQMNSLRAEDTAVYYCARVPYSGSYFDYWGQGTLVTVSS

SEQ ID NO: 1118 - protein, 117 aa, synthetic construct
EVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCTNYRYNWNDYWGQGTLVTVSS

SEQ ID NO: 1119 - protein, 117 aa, synthetic construct
EVQLVESGGGLVKPGGSLRLSCAASGFTFSYYSMNWVRQAPGKCLEWVSSISSSSTYIYYADSVKGRFTISRDNAKNSLYLQMNSLRAEDTAVYYCANYRYNWNDYWGQGTLVTVSS

SEQ ID NO: 1120 - protein, 121 aa, synthetic construct
EVQLVESGGGLVKPGGSLRLSCAASGFTFSSYSMNWVRQAPGKCLEWVSYISSSSRYIYYADSVKGRFTISRDNAKNSLYLQMNSLRAEDTAVYYCAREGPNWGTHAFDIWGQGTMVTVSS

SEQ ID NO: 1121 - protein, 117 aa, synthetic construct
EVQLVESGGALVKPGGSLRLSCAASGFTFSRYSMNWVRQAPGKCLEWVSSISSSSSYIYYADSVKGRFTISRDNAKNSLYLQMNSLRAEDTAVYYCANYRYNWNDYWGQGTLVAVSS

SEQ ID NO: 1122 - protein, 117 aa, synthetic construct
EVQLVESGGGLVKPGGSLRLSCSASGFTFSSYSMNWVRQAPGKCLEWVSSISRSSSYIFYADSVKGRFTISRDNAKNSLYLQMNSLRAEDTAVYYCARERRNGFDYWGQGTLVTVSS

SEQ ID NO: 1123 - protein, 117 aa, synthetic construct
EVQLVESGGGLVKPGGSLRLSCAASGFTFSRYSMNWVRQAPGKCLEWVSSISRSSSYIYYADSVKGRFTISRDNAKNSLYLQMNSLRAEDTAVYYCANYRYNWNDYWGQGTLVTVSS

SEQ ID NO: 1124 - protein, 117 aa, synthetic construct
EVQLVESGGGLVKPGGSLRLSCAASGFTFSRYSMNWVRQAPGKCLEWVSSISSSSSYIYYADSVKGRFTISRDNAKNSLYLQMNSLRAEDTAVYYCARAVYNYFDQWGQGTLVTVSS

SEQ ID NO: 1125 - protein, 121 aa, synthetic construct
EVQLVESGGGLVKPGGSLRLSCAASGFTFSSYSMNWVRQAPGKCLEWVSYISRSSTYIYYADSMKGRFTISRDNAKNSLYLQMNSLRAEDTAVYYCAREGPNWGTHAFDIWGQGTMVTVSS

SEQ ID NO: 1126 - protein, 118 aa, synthetic construct
EVQLVESGGGLVKPGGSLRLSCAASGLTFSSYSMNWVRQAPGKCLEWVSSISSSSSYIYYADSVKGRFTISRDNAKNSLYLQMNSLRAEDTAVYYCARERRELAFDIWGQGTMVTVSS

SEQ ID NO: 1127 - protein, 118 aa, synthetic construct
EVQLVESGGGLVKPGGSLRLSCAASGITFSSYSMNWVRQAPGKCLEWVSSISSSSSYIYYADSVKGRFTISRDNAKNSLHLQMNSLRAEDTAVYYCARERRELSFDYWGQGTLVTVSS

SEQ ID NO: 1128 - protein, 121 aa, synthetic construct
EVQLVESGRGLVNPGGSLRLSCAASGFTFSSYSMNWVRQAPGKCLEWVSYISSSSNYIYYADSVKGRFTISRDNAKNSLYLQMNSLRAEDTAVYYCAREGPNWGTHAFDIWGQGTMVTVSS

SEQ ID NO: 1129 - protein, 117 aa, synthetic construct
EVQLVESGGGLVNPGGSLRLSCAASGFTFSRYSMNWVRQAPGKCLEWVSSISSSSSYIYYADSVKGRFTISRDNAKNSLYLQMNSLRAEDTAVYYCANYRYNWNDYWGQGTLVTVSS

SEQ ID NO: 1130 - protein, 118 aa, synthetic construct
EVQLVESGGGLVKPGGSLRLSCAASGITFSSYSMNWVRQAPGKCLEWVSSISSSSSYIYYADSVKGRFTISRDNAKNSLHLQMHSLRAEDTAVYYCARERRELSFDYWGQGTLVTVSS

SEQ ID NO: 1131 - protein, 117 aa, synthetic construct
EVQLVESGGGLVKPGGSLRLSCAASGFTFSRYSMNWVRQAPGKCLEWVSSISSSSSYISYADSVKGRFTISRDNAKNSLFLQMNSLRAEDTAVYYCARVSYNSFDYWGQGTLVTVSS

SEQ ID NO: 1132 - protein, 117 aa, synthetic construct; identical to SEQ ID NO: 1124

SEQ ID NO: 1133 - protein, 117 aa, synthetic construct; identical to SEQ ID NO: 1129

SEQ ID NO: 1134 - protein, 117 aa, synthetic construct
EVQLVESGGGLVKPGGSLRLSCAASGFTFSSYSMNWVRQAPGKCLEWVSSISRSSSYIFYADSVKGRFTISRDNAKNSLYLQMNSLRAEDTAVYYCARERRNGFDYWGQGTLVTVSS

SEQ ID NO: 1135 - protein, 118 aa, synthetic construct
EVQLVQSGAEVKKPGESLRISCKGSGYSFTTYWIAWVRQMPGKCLEWMGFIYPGDSDTIYSPSFQGQVTISADKSISTAYLQWSSLRASDTAMYYCARQGYLDAFDIWGQGTMVTVSS

SEQ ID NO: 1136 - protein, 117 aa, synthetic construct
EVQLVESGGGLVKPGGSLRLSCAASGFTFSRYSMNWVRQAPGKCLEWVSSISSSSRYIYYADSVKGRFTISRDNANNSLYLHMNSLRAEDTAVYYCARALYNYFDYWGQGTLVTVSS

SEQ ID NO: 1137 - protein, 117 aa, synthetic construct
EVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCANYRYNWNDYWGQGTLVTVSS

SEQ ID NO: 1138 - protein, 118 aa, synthetic construct
EVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARERRDLAFDIWGQGTMVTVSS

SEQ ID NO: 1139 - protein, 121 aa, synthetic construct; identical to SEQ ID NO: 1125

SEQ ID NO: 1140 - protein, 118 aa, synthetic construct; identical to SEQ ID NO: 1127

SEQ ID NO: 1141 - protein, 118 aa, synthetic construct; identical to SEQ ID NO: 1130

SEQ ID NO: 1142 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1143 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 1144 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1145 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 1146 - protein, 20 aa, synthetic construct
GGGGSGGGGSGGGGSGGGGS

SEQ ID NO: 1147 - protein, 25 aa, synthetic construct
  VARIANT 1..25: note = This sequence may encompass 1-5 SSSSG repeating units
SSSSGSSSSGSSSSGSSSSGSSSSG

SEQ ID NO: 1148 - protein, 25 aa, synthetic construct
  VARIANT 1..25: note = This sequence may encompass 1-5 GSSGG repeating units
GSSGGGSSGGGSSGGGSSGGGSSGG

SEQ ID NO: 1149 - protein, 25 aa, synthetic construct
  VARIANT 1..25: note = This sequence may encompass 1-5 GGSGG repeating units
GGSGGGGSGGGGSGGGGSGGGGSGG

SEQ ID NO: 1150 - protein, 446 aa, synthetic construct
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 1151 - protein, 218 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1152 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1153 - protein, 486 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1154 - protein, 484 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSGGGGSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKTSGPGGPDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1155 - protein, 247 aa, synthetic construct
EVQLVESGEGLVQPGGSLRLSCAASGFTFSSYAMHWVRQAPGKCLEYVSAISSNGGRTYYADSVKGRFTISRDNSKNTLYLQMGSLRAEDMAVYYCARGGSLDYWGQGTLVTVSSGGGGSGGGGSGGGGSGGGGSDIVMTQTPLSSPVTPGQPASISCRSSQSLVHSDGNTYLNWLQQRPGQPPRLLIYKISDRFSGVPDRFSGSGAGTDFTLKIGRVEAEDVGVYYCMQSTQFPITFGCGTRLEIK

SEQ ID NO: 1156 - protein, 248 aa, synthetic construct
EVQLVESGGGLVKPGGSLRLSCAASGLTFSTYSMNWVRQAPGKCLEWVSSISSSGSYVYYADSVKGRFTMSRDNAKNSLYLQMNSLRAEDTAVYYCARERRELLFDYWGQGTLVTVSSGGGGSGGGGSGGGGSGGGGSNFMLTQPHSVSESPGKTVTISCTRSSGSIASNYVQWYQQRPGSSPTSVIYEDNQRPSGVPDRFSGSIDSSSNSASLTISGLKTEDEADYYCQSYDRSNRVFGCGTKLTVL

SEQ ID NO: 1157 - protein, 247 aa, synthetic construct
EVQLVESGGGLVKPGGSLRLSCAASGFTFSSYSMNWVRQAPGKCLEWVSSISRSSSYIYYADSVKGRFTLSRDNAKNSLYLQMNSLRGEDTAIYYCGRDGKGAIFLDYWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTHSSGNHRVFGCGTKLTVL

SEQ ID NO: 1158 - protein, 247 aa, synthetic construct
EVQLVESGGGLVQPGGSLRLSCAASGFTFSSYSMNWVRQAPGKCLEWVSYISSSSSTIYYADSVKGRFTISRDNAKNSLYLQMNSLRDEDTAVYYCARDGPVDYGMDVWGQGTTVTVSSGGGGSGGGGSGGGGSGGGGSSYELTQPPSVSVSPGQTARITCSGDALPKQYTYWYQQKPGQAPVLVIYKDSERPSGIPERFSGSSSGTTVTLTISGVQAEDEADYYCQSADSSGTYRVFGCGTKLTVL

SEQ ID NO: 1159 - protein, 248 aa, synthetic construct
QVQLVQSGAEVKKPGASLKVSCKASGYTFTSYYMHWVRQAPGQCLEWMGIINPSNGNTISAQKFQGRVTMTRDTSTSTVYMEMSSLRSEDTAVYYCVRSWGYYVMDVWGQGTTVTVSSGGGGSGGGGSGGGGSGGGGSQTVVTQEPSFSVSPGGTVTLTCGLNSGSVSTSYYPSWYQQTPGQTPRTLIYSTNTRSSGVPDRFSGSILGNKAALTITGAQADDESDYYCVLYMGSGIWVFGCGTKLTVL

SEQ ID NO: 1160 - protein, 248 aa, synthetic construct
EVQLVQSGAEVKKPGESLKISCKGSGYSFISYWIAWVRQMPGKCLEWMGIIYPGDSETRYRPSFQGQVTISADKSINTAYLQWSSLKAADTAMYYCARQGAYDGFDIWGQGTMVTVSSGGGGSGGGGSGGGGSGGGGSQSVLTQPPSASGTPGQRVTISCSGSSSNIGSNYVYWYQQLPGTAPKLLIYSNNQRPSGVPDRFSGSKSGTSASLAISGLRSEDEADYYCEAWDDSLSGPVFGCGTKLTVL

SEQ ID NO: 1161 - protein, 253 aa, synthetic construct
EVQLVQSGAEVKKPGESLKISCKGSGYSFTNYWIGWVRQMPGKCLEWMGIIYPADSDTTYSPSFQGQVTISADKSITTAYLQWSSLKASDTAMYYCARHGGSYILDAFDIWGQGTMVTVSSGGGGSGGGGSGGGGSGGGGSDIVMTQSPLSLPVTPGEPASISCRSSQSLLHSNGYNYLDWYLQKPGQSPQLLIYLGSNRASGVPDRFSGSGSGTDFTLKISRVEAEDVGFYYCMQPLQTPYTFGCGTKLEIK

SEQ ID NO: 1162 - protein, 248 aa, synthetic construct
EVHLVESGGGLVKPGGSLRLSCAASGFTFSRYSMNWVRQAPGKCLEWVSSISRSSSYIYYADSVKGRFTISRDNAKNSLYLQMNSLRAEDTAVYYCAREPRTLGMDYWGQGILVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 1163 - protein, 245 aa, synthetic construct
EVQLVESGGGLVQRGGSLRLSCAASGFTFSNYWMSWVRQAPGKCLEWVANIKEDGSEKYYVDSVKGRFTISRDNAKHSLYLQMNSLRAEDTAVYYCARERRTGYFDYWGQGTLVTVSSGGGGSGGGGSGGGGSGGGGSDIQMTQSPSTLSASVGDRVTITCRASQSISSWLAWFQQKTGKAPKLLIYKASSLESGVPSRFSGSGSGTEFTLTISSLQPDDFATYYCQQYISYSWTFGCGTKVEIK

SEQ ID NO: 1164 - protein, 245 aa, synthetic construct
EVQLVESGGGLVQPGGSLRLSCAASGFSFSSYSMNWVRQAPGKCLEWVSHIRSSTYSIYYADSVMGRFTISRDNAKNSLYLQMNSLRDEDTAVYFCARAKTGGDAFDIWGQGTMVTVSSGGGGSGGGGSGGGGSGGGGSDIQMTQSPSTLSASVGDRVTITCRASQSISSWLAWYQQKPGKAPKLLIHKASSLESGVPSRFSGSGSGTEFTLTISSLQPDDFATYYCQQYNSYWTFGCGTKVEIK

SEQ ID NO: 1165 - protein, 254 aa, synthetic construct
EVQLLESGGGLVQPGGSLRLSCAASGFTFSYYAMSWVRQAPGKCLEWVSAISGSGGSTYYADSVKGRFTISRDNSKNTLYLQMNSLRAEDTAVYYCAKEGIVGARAEYFQHWGQGTLVTVSSGGGGSGGGGSGGGGSGGGGSDVVMTQSPLSLPVTLGQPASISCRSSQSLVYSDGNTYLTWFQQRPGQSPRRLIYKVSNRDSGVPDRFSGSGSGTDFTLKISRVEAEDVEIYYCMQGTHWPLTFGCGTKVEIK

SEQ ID NO: 1166 - protein, 252 aa, synthetic construct
EVQLLESGGGLVQPGGSLRLSCAASGFTFSYYAMSWVRQAPGKCLEWVSAISGSGGSTYYADSVKGRFTISRDNSKNTLYLQMNSLRAEDTAVYYCAKEGIVGARAEYFQHWGQGTLVTVSSGGGGSGGGGSGGGGSGGGGSQSVLTQPPSASGTPGQRVTISCSGSSSNIGSNTVNWYQQFPGTAPKLLIYSNNQRPSGVPDRFSGSKSGTSASLAISGLQSEDEADYYCAPWDDSLNGVVFGCGTKLTVL

SEQ ID NO: 1167 - protein, 247 aa, synthetic construct
EVQLVESGGGLVKPGGSLRLSCAASGFTFSSYSMNWVRQAPGKCLEWVSSVNSRGSYIYYADSVKGRFTISRDNAKDSLYLQMNSLRADDTAVYYCARDRRELLLDYWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNHSWVFGCGTKLTVL

SEQ ID NO: 1168 - protein, 249 aa, synthetic construct
EVQLVQSGAEVKKPGESLKISCKGSEYSFTTYWIAWVRQMPGKCLEWMGIIYPGDSDTTYSPSFQGQVTISADKSISIAFLQWSSLKASDTAMYYCARQGELDAFDIWGQGTMVTVSSGGGGSGGGGSGGGGSGGGGSQSALTQPASVSGSPGQSITISCTGTSSDVGSYNLVSWYQQHPGKAPKLMIYEDIRRPSGVSNRFSASKSGDTASLTISGLQAEDEADYYCSSYAGSTTFAIFGCGTKLTVL

SEQ ID NO: 1169 - protein, 251 aa, synthetic construct
EVQLVESGGGLVQPGGSLRLSCAASGFTFSTYWMSWVRQAPGKCLEWVANIKQDGSEKYYVDSVKGRFTISRDNAKNSLYLQMNSLRAEDTAVYYCATGSFDYWGQGTLVTVSSGGGGSGGGGSGGGGSGGGGSQPVLTQPSSHSASSGASVRLTCMLSSGFSVGDFWIRWYQQKPGNPPRYLLFYHSDSNKGQGSGVPSRFSGSNDASANAGILRISGLQPEDEADYYCGTWHSNSKTWVFGCGTKLTVL

SEQ ID NO: 1170 - protein, 245 aa, synthetic construct
EVQLVESGGGLVQPGGSLRLSCAASGFTFSSYWMSWVRQAPGKCLEWVANIKEDGSEKYYVDSVKGRFTISRDNAKNSLYLQMNSLRAEDTAVYYCARERRTGYLDYWGQGTLVTVSSGGGGSGGGGSGGGGSGGGGSDIQMTQSPSTLSASVGDRVTITCRASQSISSWLAWYQQKPGKAPNLLIYKASSLESGVPSRFSGSGSGTEFTLTISSLQPDDFATYYCQQYKSYSWTIGCGTKVEIK

SEQ ID NO: 1171 - protein, 249 aa, synthetic construct
EVQLVESGGGLVQPGGSLRLSCAASGFTFSRYSMNWVRQAPGKCLEWVSYISRSSTTIYYADSVKGRFTISRDNAKNSLYLQMNSLRDEDAAVYYCARERWELLYFDYWGQGTLVTVSSGGGGSGGGGSGGGGSGGGGSNFMLTQPHSVSESPGKTVTISCTRSSGSIASKYVQWYQQRPGSSPTTVIYEDNQRPSGVPDRFSGSIDSSSNSASLTISGLKTEDEADYYCQSYYSNNRVFGCGTKLTVL

SEQ ID NO: 1172 - protein, 248 aa, synthetic construct
EVQLVESGGGLVKPGGSLRLSCSASGFTFSTYTMNWVRQAPGKCLEWVSSISKSSSYIYYTDSVKGRFTISRDNAKNSLYLQMNSLRAEDTAVYYCARERRTLAFDIWGQGTMVTVSSGGGGSGGGGSGGGGSGGGGSNFMLTQPHSVSESPGKTVTISCTRSSGSIASNYVQWYQQRPGSSPTTVIYEDNQRSSGVPDRFSGSIDSSSNSASLTISGLKTEDEADYYCQSYDRSNRVFGCGTKLTVL

SEQ ID NO: 1173 - protein, 247 aa, synthetic construct
EVQLVESGGGLVKPGGSLRLSCAASGFTFSYYSMNWVRQAPGKCLEWVSSISRSTTYIYYADSVKGRFTISRDNAKNSLYLQMNSLRAEDTAVYYCARDGKGAIFLDYWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNHRVFGCGTKLTVL

SEQ ID NO: 1174 - protein, 247 aa, synthetic construct
EVQLVESGGGLVKPGGSLRLSCAASGFTFSSYTMNWVRQAPGKCLEWVSSITTSSSYIYYADSVKGRFTISRDNAKNSLYLQMNSLRAEDTAVYYCARVTFDAFDIWGQGTMVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 1175 - protein, 246 aa, synthetic construct
EVQLVESGGGLVKPGGSLRLSCVASGFTFSRYSMNWVRQAPGKCLEWVSSISSSSRYTYYADSLKGRFIMSRDNAKNSLFLQMNSLRAEDTAVYYCARNLYNALDYWGQGTLVTVSSGGGGSGGGGSGGGGSGGGGSFMLTQPHSVSESPGKTVTISCTRSSGSIASNYVQWYQQRPGSSPTTVIYEDNQRPSGVPDRFSGSIDSSSNSASLTISGLQTEDEADYYCQSYDSSNRVFGCGTKLTVL

SEQ ID NO: 1176 - protein, 255 aa, synthetic construct
EVQLVESGGGLVKPGGSLRLSCAASGFTFSSYTMNWVRQAPGKCLEWVSSISTSSRYIYYTDSVKGRFTISRDDAKNSLYLQMNSLRAEDTAVYYCTRNWGYDVFDIWGQGTMVIVSSGGGGSGGGGSGGGGSGGGGSQPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKTQVFGCGTKLTVL

SEQ ID NO: 1177 - protein, 248 aa, synthetic construct
EVQLVESGGGLVKPGGSLRLSCAASGFTFSSYSMNWVRQAPGKCLEWVSSISRSGTYIYYADSVKGRFTISRDNAKNSLYLQMNSLRAEDTAVYYCARERWELLYFDYWGQGTLVTVSSGGGGSGGGGSGGGGSGGGGSQTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAYVFGCGTKVTVL

SEQ ID NO: 1178 - protein, 248 aa, synthetic construct
EVQLVESGGGLVKPGGSLRLSCAASGFTFSSYSMNWVRQAPGKCLEWVSSISRSSGYIFYADSVKGRFTISRDNAKNSLYLQMNSLRAEDTAVYYCVRERVGAGFDYWGQGTLVTVSSGGGGSGGGGSGGGGSGGGGSNFMLTQPHSVSESPGKTVAISCTRSSGSIASNYVQWYQQRPGSSPTAVIYEDNQRPSGVPDRFSGSIDSSSNSAFLTISGLKTEDEADYYCQSYDSSNRVFGCGTKLTVL

SEQ ID NO: 1179 - protein, 248 aa, synthetic construct
QVQLQESGPGLVKPSETLSLTCTVSGGSISSYYWSWIRQPAGKCLEWIGRIYTSGSTKYNPSLKSRVTMSVDTSKNQFSLKLISVTAADTAVYFCARERIRDLYFDYWGQGTLVTVSSGGGGSGGGGSGGGGSGGGGSNFMLTQPHSVSESPGKTVTISCTRTSGSIASNYVQWYQQRPGSSPTTVIYEDNQRPSGVPDRFSGSIDSSSNSASLTISGLKTEDEAAYYCQSYDRSNRVFGCGTKLTVL

SEQ ID NO: 1180 - protein, 249 aa, synthetic construct
EVQVAESGGGLVKPGGSLRLSCAASGFTFSRYSMNWVRQAPGKCLEWVSSISSSGSYTYYADSVKGRFTISRDNAKNSLYLQMNSLRAEDTAVYYCVRERGKGAGMDVWGQGTTVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 1181 - protein, 247 aa, synthetic construct
QVQLQQWGAGLLKPSETLSLTCAVYGGSFSGYYWSWIRQPPGKCLEWIGEIQHSGSTNYNPSLKSRVTISVDTSKNQFSLKLSSVTAADTAVYYCAREYYYYGMDVWGQGTTVTVSSGGGGSGGGGSGGGGSGGGGSQSVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGVVFGCGTKLTVL

SEQ ID NO: 1182 - protein, 248 aa, synthetic construct
QVQLQESGPGLVKPSETLSLTCTVSGGSISSYYWSWIRQPAGKCLEWIGRIYASGSTYYNPSLKSRVTMSVDTSKNQFSLKLSSVTAADTAVYYCAREKRSRLYFDYWGQGTLVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEGDYYCQSYDRSNRVFGCGTKLTVL

SEQ ID NO: 1183 - protein, 247 aa, synthetic construct
EVQLVESGGGLVKPGGSLRLSCAASGFTFSSSIMNWVRQAPGKCLEWVSSISGSSSYIYYADSVKGRFTISRDNAKNSQYLQMKSLRAEDTAVYYCARERWNLLYFDYWGQGTLVTVSSGGGGSGGGGSGGGGSGGGGSSYELTQPPSVSVSPGQTARITCSGDALPKQYAYWYQQKPGQAPVLVIYKDSERPSGIPERFSGSSSGTTVTLTISGVQAEDEADYYCQSADSSGTYVVFGCGTKLTVL

SEQ ID NO: 1184 - protein, 248 aa, synthetic construct
QVQLQESGPGLVKPSETLSLTCTVSGGSINSYYWSWIRQPAGKCLEWIGRIYTSGSTNYNPSLKSRVTMSVDTSKKQFSLKLSSVTAADTAVYYCARERISRLYFDYWGQGILVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDRSNRVFGCGTKLTVL

SEQ ID NO: 1185 - protein, 249 aa, synthetic construct
EVQLVESGGGLVKPGGSLRLSCAASGFTFSTYIMNWVRQAPGKCLEWVSSISSSRSYIYYADSVKGRFTISRDNAKNSLYLQMNSLRAEDTAVYYCAREDRYRLFFDYWGQGTLVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 1186 - protein, 246 aa, synthetic construct
EVQLVESGGGLVKPGGSLRLSCAASGFTFSSYSMKWVRQAPGKCLEWVSSISSSSSYIYYADSVKGRFTISRDNAKNSLYLQMNSLRAEDTAVYYCARDRGELTLWYWGQGTLVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNHWVFGCGTKLTVL

SEQ ID NO: 1187 - protein, 246 aa, synthetic construct
EVQLVESGGGLVKPGGSLRLSCAASGFTFSNYYMNWVRQAPGKCLERVSSISSSSNYIYYADSVKGRFTISRDNAKNSLYLQMNSLRAEDTAVYYCARDRRELPLDYWGQGTVVTVSSGGGGSGGGGSGGGGSGGGGSSYELTQPPSVSVSPGQTARITCSGDALPKKYAYWYQQKSGQAPVLVIYEDSKRPSGIPERFSGSSSGTMATLTISGAQVEDEADYYCYSTDSSGNHWVFGCGTKLTVL

SEQ ID NO: 1188 - protein, 252 aa, synthetic construct
EVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARDGELGIGTYFDYWGQGTLVTVSSGGGGSGGGGSGGGGSGGGGSQSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGCGTKLTVL

SEQ ID NO: 1189 - protein, 249 aa, synthetic construct
EVQLVESGGGLVKPGGSLRLSCAASGFTVSNYSMNWVRQAPGKCLEWVSSISRSSSYIYYADSVKGRFTISRDNAKNSLFLQMNSLRAEDTAVYYCARERWELLYFDYWGQGTLVTVSSGGGGSGGGGSGGGGSGGGGSQTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGIWVFGCGTKLTVL

SEQ ID NO: 1190 - protein, 250 aa, synthetic construct
QVQLQESGPGLVKPSQTLSLTCTVSGGSISSGGYYWSWIRQHPGKCLEWIGYIYYSGSTYYNPSLKSRVTISVDTSKDQFSLKLSSVTAADTAVYYCARERKTSLPFDIWGQGTMVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEAYYYCQSFDSSNRVFGCGTKLTVL

SEQ ID NO: 1191 - protein, 247 aa, synthetic construct
EVQLVESGGGLVKPGGSLRLSCAASGFTFSSYSMNWVRQAPGKCLEWVSSISRRSGYIYYADSVKGRFTLSRDNAKKSLYLQMNSLRAEDTAVYYCARDLNWAFDYWGQGTLVTVSSGGGGSGGGGSGGGGSGGGGSNFMLTQPHSVSESPGKTVTISCTRSSGSIASNYVQWYQQRPGSSPTTVIYEDNHRPSGVPDRFSGSIDSSSNSASLTIAGLKTEDEADYYCQSYDSSNVVFGCGTKLTVL

SEQ ID NO: 1192 - protein, 247 aa, synthetic construct
EVQLVESGGGLVKPGGSLRLSCAASGFTFSSYSMNWVRQAPGKCLEWVSSISRRSGYIYYADSVKGRFTLSRDNAKKSLYLQMNSLRAEDTAVYYCARDLNWAFDYWGQGTLVTVSSGGGGSGGGGSGGGGSGGGGSNFMLTQPHSVSESPGKTVTISCTRSSGSIASNYVQWYQQRPGSSPTTVIYEDNHRPSGVPDRFSGSIDSSSNSASLTIAGLKTEDEADYYCQSSDSSNVVFGCGTKLTVL

SEQ ID NO: 1193 - protein, 249 aa, synthetic construct
EVQLVESGGGLVKFGGSLRLSCAASGFTFSRYSMNWVRQAPGKCLEWVSSISSSSSYKYYADSVKGRFTISRDNAKNSLYLQMNSLRAEDTAVYYCAREGGNSHGMDVWGQGTTVTVSSGGGGSGGGGSGGGGSGGGGSNFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVL

SEQ ID NO: 1194 - protein, 245 aa, synthetic construct
EVQLVESGGGLVQPGGSLRLSCAASGFTFSRYWMSWVRQAPGKCLEWVANIKEDGSEKYYVDSVKGRFTISRDNAKNSLYLQMNSLRAEDTAVYFCARERRTGYFDYWGQGTLVTVSSGGGGSGGGGSGGGGSGGGGSDIQMTQSPSTLSASVGDRVTITCRASQSISSWLAWYQQKPGKAPNLLIYKASSLESGVPSRFSGSGSGTEFTLTISSLQPDDFATYYCQQYNSYSWTFGCGTKVEIK

SEQ ID NO: 1195 - protein, 247 aa, synthetic construct
EVQLVESGGGLVQPGGSLRLSCAASGFTFSSYWMSWVRQAPGKCLEWVSSISSRRGYKYHADSVKGRFTISRDNAKNSLYLQMNSLRAEDTAVYYCARVPYSGSYFDYWGQGTLVTVSSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLAPEDFAVYYCQQYGSSRWSFGCGTKVEIK

SEQ ID NO: 1196 - protein, 247 aa, synthetic construct
DIVMTQTPLSSPVTPGQPASISCRSSQSLVHSDGNTYLNWLQQRPGQPPRLLIYKISDRFSGVPDRFSGSGAGTDFTLKIGRVEAEDVGVYYCMQSTQFPITFGCGTRLEIKGGGGSGGGGSGGGGSGGGGSEVQLVESGEGLVQPGGSLRLSCAASGFTFSSYAMHWVRQAPGKCLEYVSAISSNGGRTYYADSVKGRFTISRDNSKNTLYLQMGSLRAEDMAVYYCARGGSLDYWGQGTLVTVSS

SEQ ID NO: 1197 - protein, 248 aa, synthetic construct
NFMLTQPHSVSESPGKTVTISCTRSSGSIASNYVQWYQQRPGSSPTSVIYEDNQRPSGVPDRFSGSIDSSSNSASLTISGLKTEDEADYYCQSYDRSNRVFGCGTKLTVLGGGGSGGGGSGGGGSGGGGSEVQLVESGGGLVKPGGSLRLSCAASGLTFSTYSMNWVRQAPGKCLEWVSSISSSGSYVYYADSVKGRFTMSRDNAKNSLYLQMNSLRAEDTAVYYCARERRELLFDYWGQGTLVTVSS

SEQ ID NO: 1198 - protein, 247 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTHSSGNHRVFGCGTKLTVLGGGGSGGGGSGGGGSGGGGSEVQLVESGGGLVKPGGSLRLSCAASGFTFSSYSMNWVRQAPGKCLEWVSSISRSSSYIYYADSVKGRFTLSRDNAKNSLYLQMNSLRGEDTAIYYCGRDGKGAIFLDYWGQGTLVTVSS

SEQ ID NO: 1199 - protein, 247 aa, synthetic construct
SYELTQPPSVSVSPGQTARITCSGDALPKQYTYWYQQKPGQAPVLVIYKDSERPSGIPERFSGSSSGTTVTLTISGVQAEDEADYYCQSADSSGTYRVFGCGTKLTVLGGGGSGGGGSGGGGSGGGGSEVQLVESGGGLVQPGGSLRLSCAASGFTFSSYSMNWVRQAPGKCLEWVSYISSSSSTIYYADSVKGRFTISRDNAKNSLYLQMNSLRDEDTAVYYCARDGPVDYGMDVWGQGTTVTVSS

SEQ ID NO: 1200 - protein, 248 aa, synthetic construct
QTVVTQEPSFSVSPGGTVTLTCGLNSGSVSTSYYPSWYQQTPGQTPRTLIYSTNTRSSGVPDRFSGSILGNKAALTITGAQADDESDYYCVLYMGSGIWVFGCGTKLTVLGGGGSGGGGSGGGGSGGGGSQVQLVQSGAEVKKPGASLKVSCKASGYTFTSYYMHWVRQAPGQCLEWMGIINPSNGNTISAQKFQGRVTMTRDTSTSTVYMEMSSLRSEDTAVYYCVRSWGYYVMDVWGQGTTVTVSS

SEQ ID NO: 1201 - protein, 248 aa, synthetic construct
QSVLTQPPSASGTPGQRVTISCSGSSSNIGSNYVYWYQQLPGTAPKLLIYSNNQRPSGVPDRFSGSKSGTSASLAISGLRSEDEADYYCEAWDDSLSGPVFGCGTKLTVLGGGGSGGGGSGGGGSGGGGSEVQLVQSGAEVKKPGESLKISCKGSGYSFISYWIAWVRQMPGKCLEWMGIIYPGDSETRYRPSFQGQVTISADKSINTAYLQWSSLKAADTAMYYCARQGAYDGFDIWGQGTMVTVSS

SEQ ID NO: 1202 - protein, 253 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGFYYCMQPLQTPYTFGCGTKLEIKGGGGSGGGGSGGGGSGGGGSEVQLVQSGAEVKKPGESLKISCKGSGYSFTNYWIGWVRQMPGKCLEWMGIIYPADSDTTYSPSFQGQVTISADKSITTAYLQWSSLKASDTAMYYCARHGGSYILDAFDIWGQGTMVTVSS

SEQ ID NO: 1203 - protein, 248 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVLGGGGSGGGGSGGGGSGGGGSEVHLVESGGGLVKPGGSLRLSCAASGFTFSRYSMNWVRQAPGKCLEWVSSISRSSSYIYYADSVKGRFTISRDNAKNSLYLQMNSLRAEDTAVYYCAREPRTLGMDYWGQGILVTVSS

SEQ ID NO: 1204 - protein, 245 aa, synthetic construct
DIQMTQSPSTLSASVGDRVTITCRASQSISSWLAWFQQKTGKAPKLLIYKASSLESGVPSRFSGSGSGTEFTLTISSLQPDDFATYYCQQYISYSWTFGCGTKVEIKGGGGSGGGGSGGGGSGGGGSEVQLVESGGGLVQRGGSLRLSCAASGFTFSNYWMSWVRQAPGKCLEWVANIKEDGSEKYYVDSVKGRFTISRDNAKHSLYLQMNSLRAEDTAVYYCARERRTGYFDYWGQGTLVTVSS

SEQ ID NO: 1205 - protein, 245 aa, synthetic construct
DIQMTQSPSTLSASVGDRVTITCRASQSISSWLAWYQQKPGKAPKLLIHKASSLESGVPSRFSGSGSGTEFTLTISSLQPDDFATYYCQQYNSYWTFGCGTKVEIKGGGGSGGGGSGGGGSGGGGSEVQLVESGGGLVQPGGSLRLSCAASGFSFSSYSMNWVRQAPGKCLEWVSHIRSSTYSIYYADSVMGRFTISRDNAKNSLYLQMNSLRDEDTAVYFCARAKTGGDAFDIWGQGTMVTVSS

SEQ ID NO: 1206 - protein, 254 aa, synthetic construct
DVVMTQSPLSLPVTLGQPASISCRSSQSLVYSDGNTYLTWFQQRPGQSPRRLIYKVSNRDSGVPDRFSGSGSGTDFTLKISRVEAEDVEIYYCMQGTHWPLTFGCGTKVEIKGGGGSGGGGSGGGGSGGGGSEVQLLESGGGLVQPGGSLRLSCAASGFTFSYYAMSWVRQAPGKCLEWVSAISGSGGSTYYADSVKGRFTISRDNSKNTLYLQMNSLRAEDTAVYYCAKEGIVGARAEYFQHWGQGTLVTVSS

SEQ ID NO: 1207 - protein, 252 aa, synthetic construct
QSVLTQPPSASGTPGQRVTISCSGSSSNIGSNTVNWYQQFPGTAPKLLIYSNNQRPSGVPDRFSGSKSGTSASLAISGLQSEDEADYYCAPWDDSLNGVVFGCGTKLTVLGGGGSGGGGSGGGGSGGGGSEVQLLESGGGLVQPGGSLRLSCAASGFTFSYYAMSWVRQAPGKCLEWVSAISGSGGSTYYADSVKGRFTISRDNSKNTLYLQMNSLRAEDTAVYYCAKEGIVGARAEYFQHWGQGTLVTVSS

SEQ ID NO: 1208 - protein, 247 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHSWVFGCGTKLTVLGGGGSGGGGSGGGGSGGGGSEVQLVESGGGLVKPGGSLRLSCAASGFTFSSYSMNWVRQAPGKCLEWVSSVNSRGSYIYYADSVKGRFTISRDNAKDSLYLQMNSLRADDTAVYYCARDRRELLLDYWGQGTLVTVSS

SEQ ID NO: 1209 - protein, 249 aa, synthetic construct
QSALTQPASVSGSPGQSITISCTGTSSDVGSYNLVSWYQQHPGKAPKLMIYEDIRRPSGVSNRFSASKSGDTASLTISGLQAEDEADYYCSSYAGSTTFAIFGCGTKLTVLGGGGSGGGGSGGGGSGGGGSEVQLVQSGAEVKKPGESLKISCKGSEYSFTTYWIAWVRQMPGKCLEWMGIIYPGDSDTTYSPSFQGQVTISADKSISIAFLQWSSLKASDTAMYYCARQGELDAFDIWGQGTMVTVSS

SEQ ID NO: 1210 - protein, 251 aa, synthetic construct
QPVLTQPSSHSASSGASVRLTCMLSSGFSVGDFWIRWYQQKPGNPPRYLLFYHSDSNKGQGSGVPSRFSGSNDASANAGILRISGLQPEDEADYYCGTWHSNSKTWVFGCGTKLTVLGGGGSGGGGSGGGGSGGGGSEVQLVESGGGLVQPGGSLRLSCAASGFTFSTYWMSWVRQAPGKCLEWVANIKQDGSEKYYVDSVKGRFTISRDNAKNSLYLQMNSLRAEDTAVYYCATGSFDYWGQGTLVTVSS

SEQ ID NO: 1211 - protein, 245 aa, synthetic construct
DIQMTQSPSTLSASVGDRVTITCRASQSISSWLAWYQQKPGKAPNLLIYKASSLESGVPSRFSGSGSGTEFTLTISSLQPDDFATYYCQQYKSYSWTIGCGTKVEIKGGGGSGGGGSGGGGSGGGGSEVQLVESGGGLVQPGGSLRLSCAASGFTFSSYWMSWVRQAPGKCLEWVANIKEDGSEKYYVDSVKGRFTISRDNAKNSLYLQMNSLRAEDTAVYYCARERRTGYLDYWGQGTLVTVSS

SEQ ID NO: 1212 - protein, 249 aa, synthetic construct
NFMLTQPHSVSESPGKTVTISCTRSSGSIASKYVQWYQQRPGSSPTTVIYEDNQRPSGVPDRFSGSIDSSSNSASLTISGLKTEDEADYYCQSYYSNNRVFGCGTKLTVLGGGGSGGGGSGGGGSGGGGSEVQLVESGGGLVQPGGSLRLSCAASGFTFSRYSMNWVRQAPGKCLEWVSYISRSSTTIYYADSVKGRFTISRDNAKNSLYLQMNSLRDEDAAVYYCARERWELLYFDYWGQGTLVTVSS

SEQ ID NO: 1213 - protein, 248 aa, synthetic construct
NFMLTQPHSVSESPGKTVTISCTRSSGSIASNYVQWYQQRPGSSPTTVIYEDNQRSSGVPDRFSGSIDSSSNSASLTISGLKTEDEADYYCQSYDRSNRVFGCGTKLTVLGGGGSGGGGSGGGGSGGGGSEVQLVESGGGLVKPGGSLRLSCSASGFTFSTYTMNWVRQAPGKCLEWVSSISKSSSYIYYTDSVKGRFTISRDNAKNSLYLQMNSLRAEDTAVYYCARERRTLAFDIWGQGTMVTVSS

SEQ ID NO: 1214 - protein, 247 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHRVFGCGTKLTVLGGGGSGGGGSGGGGSGGGGSEVQLVESGGGLVKPGGSLRLSCAASGFTFSYYSMNWVRQAPGKCLEWVSSISRSTTYIYYADSVKGRFTISRDNAKNSLYLQMNSLRAEDTAVYYCARDGKGAIFLDYWGQGTLVTVSS

SEQ ID NO: 1215 - protein, 247 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVLGGGGSGGGGSGGGGSGGGGSEVQLVESGGGLVKPGGSLRLSCAASGFTFSSYTMNWVRQAPGKCLEWVSSITTSSSYIYYADSVKGRFTISRDNAKNSLYLQMNSLRAEDTAVYYCARVTFDAFDIWGQGTMVTVSS

SEQ ID NO: 1216 - protein, 246 aa, synthetic construct
FMLTQPHSVSESPGKTVTISCTRSSGSIASNYVQWYQQRPGSSPTTVIYEDNQRPSGVPDRFSGSIDSSSNSASLTISGLQTEDEADYYCQSYDSSNRVFGCGTKLTVLGGGGSGGGGSGGGGSGGGGSEVQLVESGGGLVKPGGSLRLSCVASGFTFSRYSMNWVRQAPGKCLEWVSSISSSSRYTYYADSLKGRFIMSRDNAKNSLFLQMNSLRAEDTAVYYCARNLYNALDYWGQGTLVTVSS

SEQ ID NO: 1217 - protein, 255 aa, synthetic construct
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKTQVFGCGTKLTVLGGGGSGGGGSGGGGSGGGGSEVQLVESGGGLVKPGGSLRLSCAASGFTFSSYTMNWVRQAPGKCLEWVSSISTSSRYIYYTDSVKGRFTISRDDAKNSLYLQMNSLRAEDTAVYYCTRNWGYDVFDIWGQGTMVIVSS

SEQ ID NO: 1218 - protein, 248 aa, synthetic construct
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAYVFGCGTKVTVLGGGGSGGGGSGGGGSGGGGSEVQLVESGGGLVKPGGSLRLSCAASGFTFSSYSMNWVRQAPGKCLEWVSSISRSGTYIYYADSVKGRFTISRDNAKNSLYLQMNSLRAEDTAVYYCARERWELLYFDYWGQGTLVTVSS

SEQ ID NO: 1219 - protein, 248 aa, synthetic construct
NFMLTQPHSVSESPGKTVAISCTRSSGSIASNYVQWYQQRPGSSPTAVIYEDNQRPSGVPDRFSGSIDSSSNSAFLTISGLKTEDEADYYCQSYDSSNRVFGCGTKLTVLGGGGSGGGGSGGGGSGGGGSEVQLVESGGGLVKPGGSLRLSCAASGFTFSSYSMNWVRQAPGKCLEWVSSISRSSGYIFYADSVKGRFTISRDNAKNSLYLQMNSLRAEDTAVYYCVRERVGAGFDYWGQGTLVTVSS

SEQ ID NO: 1220 - protein, 248 aa, synthetic construct
NFMLTQPHSVSESPGKTVTISCTRTSGSIASNYVQWYQQRPGSSPTTVIYEDNQRPSGVPDRFSGSIDSSSNSASLTISGLKTEDEAAYYCQSYDRSNRVFGCGTKLTVLGGGGSGGGGSGGGGSGGGGSQVQLQESGPGLVKPSETLSLTCTVSGGSISSYYWSWIRQPAGKCLEWIGRIYTSGSTKYNPSLKSRVTMSVDTSKNQFSLKLISVTAADTAVYFCARERIRDLYFDYWGQGTLVTVSS

SEQ ID NO: 1221 - protein, 249 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVLGGGGSGGGGSGGGGSGGGGSEVQVAESGGGLVKPGGSLRLSCAASGFTFSRYSMNWVRQAPGKCLEWVSSISSSGSYTYYADSVKGRFTISRDNAKNSLYLQMNSLRAEDTAVYYCVRERGKGAGMDVWGQGTTVTVSS

SEQ ID NO: 1222 - protein, 247 aa, synthetic construct
QSVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGVVFGCGTKLTVLGGGGSGGGGSGGGGSGGGGSQVQLQQWGAGLLKPSETLSLTCAVYGGSFSGYYWSWIRQPPGKCLEWIGEIQHSGSTNYNPSLKSRVTISVDTSKNQFSLKLSSVTAADTAVYYCAREYYYYGMDVWGQGTTVTVSS

SEQ ID NO: 1223 - protein, 248 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEGDYYCQSYDRSNRVFGCGTKLTVLGGGGSGGGGSGGGGSGGGGSQVQLQESGPGLVKPSETLSLTCTVSGGSISSYYWSWIRQPAGKCLEWIGRIYASGSTYYNPSLKSRVTMSVDTSKNQFSLKLSSVTAADTAVYYCAREKRSRLYFDYWGQGTLVTVSS

SEQ ID NO: 1224 - protein, 247 aa, synthetic construct
SYELTQPPSVSVSPGQTARITCSGDALPKQYAYWYQQKPGQAPVLVIYKDSERPSGIPERFSGSSSGTTVTLTISGVQAEDEADYYCQSADSSGTYVVFGCGTKLTVLGGGGSGGGGSGGGGSGGGGSEVQLVESGGGLVKPGGSLRLSCAASGFTFSSSIMNWVRQAPGKCLEWVSSISGSSSYIYYADSVKGRFTISRDNAKNSQYLQMKSLRAEDTAVYYCARERWNLLYFDYWGQGTLVTVSS

SEQ ID NO: 1225 - protein, 248 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDRSNRVFGCGTKLTVLGGGGSGGGGSGGGGSGGGGSQVQLQESGPGLVKPSETLSLTCTVSGGSINSYYWSWIRQPAGKCLEWIGRIYTSGSTNYNPSLKSRVTMSVDTSKKQFSLKLSSVTAADTAVYYCARERISRLYFDYWGQGILVTVSS

SEQ ID NO: 1226 - protein, 249 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVLGGGGSGGGGSGGGGSGGGGSEVQLVESGGGLVKPGGSLRLSCAASGFTFSTYIMNWVRQAPGKCLEWVSSISSSRSYIYYADSVKGRFTISRDNAKNSLYLQMNSLRAEDTAVYYCAREDRYRLFFDYWGQGTLVTVSS

SEQ ID NO: 1227 - protein, 246 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHWVFGCGTKLTVLGGGGSGGGGSGGGGSGGGGSEVQLVESGGGLVKPGGSLRLSCAASGFTFSSYSMKWVRQAPGKCLEWVSSISSSSSYIYYADSVKGRFTISRDNAKNSLYLQMNSLRAEDTAVYYCARDRGELTLWYWGQGTLVTVSS

SEQ ID NO: 1228 - protein, 246 aa, synthetic construct
SYELTQPPSVSVSPGQTARITCSGDALPKKYAYWYQQKSGQAPVLVIYEDSKRPSGIPERFSGSSSGTMATLTISGAQVEDEADYYCYSTDSSGNHWVFGCGTKLTVLGGGGSGGGGSGGGGSGGGGSEVQLVESGGGLVKPGGSLRLSCAASGFTFSNYYMNWVRQAPGKCLERVSSISSSSNYIYYADSVKGRFTISRDNAKNSLYLQMNSLRAEDTAVYYCARDRRELPLDYWGQGTVVTVSS

SEQ ID NO: 1229 - protein, 252 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGCGTKLTVLGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARDGELGIGTYFDYWGQGTLVTVSS

SEQ ID NO: 1230 - protein, 249 aa, synthetic construct
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGIWVFGCGTKLTVLGGGGSGGGGSGGGGSGGGGSEVQLVESGGGLVKPGGSLRLSCAASGFTVSNYSMNWVRQAPGKCLEWVSSISRSSSYIYYADSVKGRFTISRDNAKNSLFLQMNSLRAEDTAVYYCARERWELLYFDYWGQGTLVTVSS

SEQ ID NO: 1231 - protein, 250 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEAYYYCQSFDSSNRVFGCGTKLTVLGGGGSGGGGSGGGGSGGGGSQVQLQESGPGLVKPSQTLSLTCTVSGGSISSGGYYWSWIRQHPGKCLEWIGYIYYSGSTYYNPSLKSRVTISVDTSKDQFSLKLSSVTAADTAVYYCARERKTSLPFDIWGQGTMVTVSS

SEQ ID NO: 1232 - protein, 247 aa, synthetic construct
NFMLTQPHSVSESPGKTVTISCTRSSGSIASNYVQWYQQRPGSSPTTVIYEDNHRPSGVPDRFSGSIDSSSNSASLTIAGLKTEDEADYYCQSYDSSNVVFGCGTKLTVLGGGGSGGGGSGGGGSGGGGSEVQLVESGGGLVKPGGSLRLSCAASGFTFSSYSMNWVRQAPGKCLEWVSSISRRSGYIYYADSVKGRFTLSRDNAKKSLYLQMNSLRAEDTAVYYCARDLNWAFDYWGQGTLVTVSS

SEQ ID NO: 1233 - protein, 247 aa, synthetic construct
NFMLTQPHSVSESPGKTVTISCTRSSGSIASNYVQWYQQRPGSSPTTVIYEDNHRPSGVPDRFSGSIDSSSNSASLTIAGLKTEDEADYYCQSSDSSNVVFGCGTKLTVLGGGGSGGGGSGGGGSGGGGSEVQLVESGGGLVKPGGSLRLSCAASGFTFSSYSMNWVRQAPGKCLEWVSSISRRSGYIYYADSVKGRFTLSRDNAKKSLYLQMNSLRAEDTAVYYCARDLNWAFDYWGQGTLVTVSS

SEQ ID NO: 1234 - protein, 249 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNRVFGCGTKLTVLGGGGSGGGGSGGGGSGGGGSEVQLVESGGGLVKFGGSLRLSCAASGFTFSRYSMNWVRQAPGKCLEWVSSISSSSSYKYYADSVKGRFTISRDNAKNSLYLQMNSLRAEDTAVYYCAREGGNSHGMDVWGQGTTVTVSS

SEQ ID NO: 1235 - protein, 245 aa, synthetic construct
DIQMTQSPSTLSASVGDRVTITCRASQSISSWLAWYQQKPGKAPNLLIYKASSLESGVPSRFSGSGSGTEFTLTISSLQPDDFATYYCQQYNSYSWTFGCGTKVEIKGGGGSGGGGSGGGGSGGGGSEVQLVESGGGLVQPGGSLRLSCAASGFTFSRYWMSWVRQAPGKCLEWVANIKEDGSEKYYVDSVKGRFTISRDNAKNSLYLQMNSLRAEDTAVYFCARERRTGYFDYWGQGTLVTVSS

SEQ ID NO: 1236 - protein, 247 aa, synthetic construct
EIVLTQSPGTLSLSPGERATLSCRASQSVSSSYLAWYQQKPGQAPRLLIYGASSRATGIPDRFSGSGSGTDFTLTISRLAPEDFAVYYCQQYGSSRWSFGCGTKVEIKGGGGSGGGGSGGGGSGGGGSEVQLVESGGGLVQPGGSLRLSCAASGFTFSSYWMSWVRQAPGKCLEWVSSISSRRGYKYHADSVKGRFTISRDNAKNSLYLQMNSLRAEDTAVYYCARVPYSGSYFDYWGQGTLVTVSS

SEQ ID NO: 1237 - protein, 5 aa, synthetic construct
GGGGS

SEQ ID NO: 1238 - protein, 25 aa, synthetic construct
  VARIANT 1..25: note = This sequence may encompass 1-5 GGGGS repeating units
GGGGSGGGGSGGGGSGGGGSGGGGS

(19) United States
(12) Patent Application Publication
(10) Pub. No.: US 2026/0167721 A1
(43) Pub. Date: Jun. 18, 2026

(54) ANTI-CD3 ANTIBODIES

(71) Applicant: Ablexis, LLC, San Diego, CA (US)

(72) Inventors: Godfrey Jonah Rainey, San Diego, CA (US); Jane Seagal, San Diego, CA (US); Arvin Akoopie, San Diego, CA (US)

(21) Appl. No.: 19/123,913

(22) PCT Filed: Oct. 25, 2023
(86) PCT No.: PCT/US2023/077780

§ 371 Date: Apr. 24, 2025

Related U.S. Application Data
(60) Provisional application No. 63/419,257, filed on Oct. 25, 2022.
(60) Provisional application No. 63/425,248, filed on Nov. 14, 2022.

(51) Int. Cl. C07K 16/28; A61P 35/00; A61P 37/02

(52) U.S. Cl. CPC C07K 16/2809; A61P 35/00; A61P 37/02; C07K 16/2803; C07K 2317/24; C07K 2317/31; C07K 2317/52; C07K 2317/565; C07K 2317/622; C07K 2317/92; C07K 2317/94

(57) ABSTRACT

The present disclosure provides anti-CD3 monoclonal antibodies and related compositions, which may be used in any of a variety of therapeutic and diagnostic methods for the treatment of diseases and disorders, such as cancer and autoimmune diseases and disorders. The present disclosure also relates to bispecific antibodies comprising anti-CD3 antibodies. The present disclosure further relates to methods of treatment using such anti-CD3 monoclonal and bispecific antibodies.

### CROSS-REFERENCE TO RELATED APPLICATIONS

This application claims priority to and benefit of U.S. Provisional Application No. 63/419,257, filed Oct. 25, 2022, and U.S. Provisional Application No. 63/425,248, filed Nov. 14, 2022, the contents of each application are hereby incorporated by reference in their entireties.

### BACKGROUND

### Technical Field

The present disclosure relates generally to anti-CD3 antibodies, compositions and methods of using same. Such antibodies are useful, for example, for targeting T-cells, e.g., as treatments for cancer or autoimmunity. Such antibodies may also be useful, for example, as targeting arms in bispecific antibodies or other multi-specific antibodies.

### Description of the Related Art

Cluster of differentiation 3 (CD3) is a multimeric protein complex comprising four distinct CD3 polypeptide chains (epsilon (ε), gamma (γ), delta (δ) and zeta (ζ)), which assemble and function as three pairs of dimers (εγ, εδ, ζζ). The assembled CD3 protein acts as a T-cell co-receptor that associates noncovalently with the T-cell receptor (TCR). The CD3 protein complex is a defining feature of the T-cell lineage, such that anti-CD3 antibodies can be used effectively as T-cell markers.

Anti-CD3 antibodies have been employed clinically for immunosuppression of autoimmune T-cell mediated diseases including organ transplant rejections and type 1 diabetes mellitus. Anti-CD3 bispecific or multi-specific antibodies represent an emerging immuno-oncology therapy. In addition to a CD3 binding arm, such bispecific or multi-specific antibodies also comprise an arm that binds to a tumor-associated antigen expressed on tumor cells and, thus, can simultaneously bind a tumor cell and a T cell. This simultaneous binding results in T-cell activation, proliferation, and the secretion of cytolytic molecules that kill the tumor cells. Some anti-CD3 binding domains deployed in some bispecific or multi-specific antibodies can stimulate the secretion of inflammatory cytokines to detectable levels. One advantage in using anti-CD3 bispecific or multi-specific antibodies compared to engineered T-cell therapy is that any T cell can serve as an effector cell, regardless of TCR specificity. Because TCR signaling does not require engagement of the antigen-binding domain of the TCR, but is initiated via CD3, anti-CD3 bispecific or multi-specific antibodies can employ all available T cells and are not limited to tumor-specific T cells. By contrast, effective immune checkpoint therapy requires the availability of tumor-specific T cells.

While an anti-CD3/anti-CD19 bispecific antibody (blinatumomab) has been approved for use in treating B-cell malignancies, other anti-CD3 bispecific antibodies have failed in early clinical trials, with the particular anti-CD3 binding domains used in those antibodies being identified as potential culprits for those failures. See, e.g., Vafa O and Trinklein N D, Front. Oncol., 2020, vol. 10, doi: 10.3389/fonc.2020.00446. Accordingly, there remains a need to identify additional anti-CD3 binding antibodies, bispecific antibodies, and multi-specific antibodies, e.g., improved efficacy or better safety profiles in patients.

### SUMMARY OF THE DISCLOSURE

The present disclosure relates to antibodies that bind CD3. More specifically, it relates to chimeric anti-CD3 antibodies generated from an AlivaMab® Mouse (Ablexis, LLC); fully human anti-CD3 antibodies produced therefrom; bispecific and multi-specific antibodies produced therefrom; compositions comprising such chimeric, human and bispecific or multi-specific antibodies; polynucleotides, vectors and host cells producing such chimeric, human and bispecific or multi-specific antibodies; and methods of making and using such chimeric, human, and bispecific or multi-specific antibodies.

One aspect of the disclosure provides an isolated anti-CD3 antibody, or an antigen-binding fragment thereof, comprising one to six complementarity determining regions (CDRs) disclosed herein. The one to six CDRs may comprise an amino acid sequence selected from the group consisting of SEQ ID NOs: 1-687.

In some embodiments, the anti-CD3 antibody, or antigen-binding fragment thereof, comprises a heavy chain CDR3 (HCDR3) disclosed herein. The HCDR3 may comprise an amino acid sequence selected from the group consisting of SEQ ID NOs: 574-687. In some embodiments, the anti-CD3 antibody, or antigen-binding fragment thereof, comprises a light chain CDR3 (LCDR3) disclosed herein. The LCDR3 may comprise an amino acid sequence selected from the group consisting of SEQ ID NOs: 227-344.

In some embodiments, the anti-CD3 antibody, or antigen-binding fragment thereof, comprises a HCDR1 disclosed herein, a HCDR2 disclosed herein, a HCDR3 disclosed herein, a LCDR1 disclosed herein, a LCDR2 disclosed herein, and a LCDR3 disclosed herein.

In some embodiments, the anti-CD3 antibody, or antigen-binding fragment thereof, comprises i) a heavy chain variable region comprising a HCDR1 disclosed herein, a HCDR2 disclosed herein, and a HCDR3 disclosed herein and ii) a light chain variable region comprising a LCDR1 disclosed herein, a LCDR2 disclosed herein, and a LCDR3 disclosed herein. The HCDR1 may comprise an amino acid sequence selected from the group consisting of SEQ ID NOs: 345-457. In some embodiments, the HCDR2 comprises an amino acid sequence selected from the group consisting of SEQ ID NOs: 458-573. The HCDR3 may comprise an amino acid sequence selected from the group consisting of SEQ ID NOs: 574-687. In some embodiments the LCDR1 comprises an amino acid sequence selected from the group consisting of SEQ ID NOs: 1-113. The LCDR2 may comprise an amino acid sequence selected from the group consisting of SEQ ID NOs: 114-226. In some embodiments, the LCDR3 comprises an amino acid sequence selected from the group consisting of SEQ ID NOs: 227-344.

In some embodiments, the anti-CD3 antibody, or antigen-binding fragment thereof, comprises the HCDR1, the HCDR2, the HCDR3, the LCDR1, the LCDR2, and the LCDR3 of an antibody disclosed herein. In some embodiments, the anti-CD3 antibody, or antigen-binding fragment thereof, comprises i) a heavy chain variable region comprising the HCDR1, the HCDR2, and the HCDR3 of an antibody disclosed herein and ii) a light chain variable region comprising the LCDR1, the LCDR2, and the LCDR3 of the same antibody. In some embodiments, the anti-CD3 antibody, or antigen-binding fragment thereof, comprises the heavy chain variable (“VH”) region of an antibody disclosed herein. The anti-CD3 antibody, or antigen-binding fragment thereof, may comprise the light chain variable (“VL”) region of an antibody disclosed herein. In some embodiments, the antibody, or antigen-binding fragment thereof, comprises the VH and VL of an antibody disclosed herein. The CDRs, VH, and/or VL may be from an antibody selected from the group consisting of ABL-CD3-1 to ABL-CD3-113, ABL-CD3-1A to ABL-CD3-113A, and ABL-CD3-85B. In some embodiments, the CDRs, VH, and/or VL may be from an antibody selected from the group consisting of ABL-CD3-18, ABL-CD3-20, ABL-CD3-21, ABL-CD3-22, ABL-CD3-27, ABL-CD3-29, ABL-CD3-31, ABL-CD3-34, ABL-CD3-36, ABL-CD3-37, ABL-CD3-39, ABL-CD3-40, ABL-CD3-41, ABL-CD3-43, ABL-CD3-44, ABL-CD3-45, ABL-CD3-47, ABL-CD3-49, ABL-CD3-52, ABL-CD3-53, ABL-CD3-56, ABL-CD3-57, ABL-CD3-61, ABL-CD3-62, ABL-CD3-63, ABL-CD3-64, ABL-CD3-65, ABL-CD3-66, ABL-CD3-68, ABL-CD3-71, ABL-CD3-72, ABL-CD3-74, ABL-CD3-76, ABL-CD3-77, ABL-CD3-79, ABL-CD3-81, ABL-CD3-85, ABL-CD3-87, ABL-CD3-88, ABL-CD3-89, ABL-CD3-18A, ABL-CD3-20A, ABL-CD3-21A, ABL-CD3-22A, ABL-CD3-27A, ABL-CD3-29A, ABL-CD3-31A, ABL-CD3-34A, ABL-CD3-36A, ABL-CD3-37A, ABL-CD3-39A, ABL-CD3-40A, ABL-CD3-41A, ABL-CD3-43A, ABL-CD3-44A, ABL-CD3-45A, ABL-CD3-47A, ABL-CD3-49A, ABL-CD3-52A, ABL-CD3-53A, ABL-CD3-56A, ABL-CD3-57A, ABL-CD3-61A, ABL-CD3-62A, ABL-CD3-63A, ABL-CD3-64A, ABL-CD3-65A, ABL-CD3-66A, ABL-CD3-68A, ABL-CD3-71A, ABL-CD3-72A, ABL-CD3-74A, ABL-CD3-76A, ABL-CD3-77A, ABL-CD3-79A, ABL-CD3-81A, ABL-CD3-85A, ABL-CD3-85B, ABL-CD3-87A, ABL-CD3-88A, and ABL-CD3-89A

The HCDR1 may comprise an amino acid sequence selected from the group consisting of SEQ ID NOs: 345-457. In some embodiments, the HCDR2 comprises an amino acid sequence selected from the group consisting of SEQ ID NOs: 458-573. The HCDR3 may comprise an amino acid sequence selected from the group consisting of SEQ ID NOs: 574-687. In some embodiments the LCDR1 comprises an amino acid sequence selected from the group consisting of SEQ ID NOs:1-113. The LCDR2 may comprise an amino acid sequence selected from the group consisting of SEQ ID NOs: 114-226. In some embodiments, the LCDR3 comprises an amino acid sequence selected from the group consisting of SEQ ID NOs: 227-344. In some embodiments, the VL region comprises an amino acid sequence selected from the group consisting of SEQ ID NOs: 688-914. The VH region may comprise an amino acid sequence selected from the group consisting of SEQ ID NOs: 915-1141.

In some embodiments, the anti-CD3 antibody, or antigen-binding fragment thereof, is human. In some embodiments, the antibody is chimeric. In certain embodiments, the antibody is selected from a single-variable domain antibody, single chain antibody, a scFv, a bispecific antibody, a multi-specific antibody, a Fab, a F(ab′)2, and a whole antibody. In some embodiments, the anti-CD3 antibody, or antigen-binding fragment thereof, is a bispecific antibody.

In some embodiments, the anti-CD3 antibody is an IgG antibody. The anti-CD3 antibody may be an IgM antibody. In some embodiments, the anti-CD3 antibody is an IgA antibody. The anti-CD3 antibody may be an IgD antibody. The anti-CD3 antibody may be an IgE antibody. In some embodiments, the anti-CD3 antibody is an IgG1 antibody. The anti-CD3 antibody may be an IgG2 antibody. In some embodiments, the anti-CD3 antibody is an IgG3 antibody. The anti-CD3 may be an IgG4 antibody. In some embodiments, the anti-CD3 antibody is a variant of an IgG antibody, such as a variant of an IgG1, an IgG2, an IgG3 or an IgG4 or combinations thereof. The anti-CD3 antibody may have reduced effector function or no effector function. In some embodiments, the anti-CD3 antibody is engineered to reduce or eliminate effector function.

An additional aspect of the disclosure provides an anti-CD3 bispecific antibody. In some embodiments, the anti-CD3 bispecific antibody further comprises a targeting antibody, or an antigen-binding fragment thereof. In some embodiments, the targeting antibody is an anti-tumor antibody, or an antigen-binding fragment thereof.

In some embodiments, the anti-CD3 antibody, or antigen-binding fragment thereof, comprises a complete heavy chain, comprising a first Fc domain, and a complete light chain. In some embodiments, the anti-CD3 antibody, or antigen-binding fragment thereof, comprises a single-chain Fv (scFv), Fab, Fab′, F(ab′)₂ or an Fv fragment operably linked to a first Fc domain. In some embodiments, the anti-CD3 antibody, or antigen-binding fragment thereof, comprises a single-chain Fv (scFv) operably linked to a first Fc domain.

In some embodiments, the anti-CD3 antibody, or antigen-binding fragment thereof, comprises a first Fc domain. In some embodiments, the anti-CD3 antibody, or antigen-binding fragment thereof, is operably linked to the first Fc domain via a first linker. In some embodiments, the first linker is an antibody hinge region. In some embodiments, the first linker is a variable length Gly-Ser linker. In some embodiments, the first linker comprises a linker selected from the group consisting of (Gly-Gly-Gly-Gly-Ser)n (SEQ ID NO: 1238), (Ser-Ser-Ser-Ser-Gly)n (SEQ ID NO: 1147), (Gly-Ser-Ser-Gly-Gly)n (SEQ ID NO: 1148), and (Gly-Gly-Ser-Gly-Gly)n (SEQ ID NO: 1149), where n is an integer between 1 and 5. In some embodiments, the first linker comprises the amino acid sequence of (Gly-Gly-Gly-Gly-Ser)n (SEQ ID NO: 1237), wherein n is 1. In some embodiments, the first linker comprises the amino acid sequence of (Gly-Gly-Gly-Gly-Ser)n (SEQ ID NO: 1146), wherein n is 4.

In some embodiments, the targeting antibody is an anti-tumor antibody. In some embodiments, the anti-tumor antibody, or antigen-binding fragment thereof, comprises a second Fc domain. In some embodiments, the anti-tumor antibody, or antigen-binding fragment thereof, is operably linked to a second Fc domain via a second linker. In some embodiments, the second linker is an antibody hinge region. In some embodiments, the second linker is a variable length Gly-Ser linker. In some embodiments, the second linker comprises a linker selected from the group consisting of (Gly-Gly-Gly-Gly-Ser)n (SEQ ID NO: 1238), (Ser-Ser-Ser-Ser-Gly)n (SEQ ID NO: 1147), (Gly-Ser-Ser-Gly-Gly)n (SEQ ID NO: 1148), and (Gly-Gly-Ser-Gly-Gly)n (SEQ ID NO: 1149), where n is an integer between 1 and 5. In some embodiments, the second linker comprises the amino acid sequence of (Gly-Gly-Gly-Gly-Ser)n (SEQ ID NO: 1146), wherein n is 1. In some embodiments, the second linker comprises the amino acid sequence of (Gly-Gly-Gly-Gly-Ser)n (SEQ ID NO: 1146), wherein n is 4.

In some embodiments, the first and second Fc domains comprises mutations to promote heterodimer formation. In some embodiments, the first Fc domain comprises a T366W mutation and the second Fc domain comprises T366S/L368A/Y407V mutations. In some embodiments, the first Fc domain comprises T366S/L368A/Y407V mutations and the second Fc domain comprises a T366W mutation. In some embodiments, the first and second Fc domains comprise KIHs-s mutations. In some embodiments, the first Fc domain comprises S354C/T366W mutations and the second Fc domain comprises Y349C/T366S/L368A/Y407V mutations. In some embodiments, the first Fc domain comprises Y349C/T366S/L368A/Y407V mutations and the second Fc domain comprises S354C/T366W mutation. In some embodiments, the first Fc domain comprises S354C/T366W mutations and the second Fc domain comprises Y349C/T366S/L368A/Y407V/H435R/Y436F mutations. In some embodiments, the first Fc domain comprises Y349C/T366S/L368A/Y407V/H435R/Y436F mutations and the second Fc domain comprises S354C/T366W mutation. In some embodiments, the first Fc domain comprises S354C/T366W/H435R/Y436F mutations and the second Fc domain comprises Y349C/T366S/L368A/Y407V mutations. In some embodiments, the first Fc domain comprises Y349C/T366S/L368A/Y407V mutations and the second Fc domain comprises S354C/T366W/H435R/Y436F mutations. Each of the foregoing mutations is according to EU numbering.

In some embodiments, the first Fc domain comprises the amino acid sequence of SEQ ID NO: 1142. In some embodiments, the first Fc domain comprises the amino acid sequence of SEQ ID NO: 1143. In some embodiments, the second Fc domain comprises the amino acid sequence of SEQ ID NO: 1142. In some embodiments, the second Fc domain comprises the amino acid sequence of SEQ ID NO: 1143. In some embodiments, the first and second Fc domains comprise the amino acid sequences of SEQ ID NOs: 1142 and 1143, respectively. In some embodiments, the first and second Fc domains comprise the amino acid sequences of SEQ ID NOs: 1143 and 1142, respectively. In some embodiments, the anti-CD3 antibody comprises a constant region comprising the amino acid sequence of SEQ ID NO: 1144. In some embodiments, the anti-CD3 antibody comprises a constant region comprising the amino acid sequence of SEQ ID NO: 1145. In some embodiments, the anti-tumor antibody comprises a constant region comprising the amino acid sequence of SEQ ID NO: 1144. In some embodiments, the anti-tumor antibody comprises a constant region comprising the amino acid sequence of SEQ ID NO: 1145. In some embodiments, the anti-CD3 antibody comprises a constant region comprising the amino acid sequence of SEQ ID NO: 1144, and the anti-tumor antibody comprises a constant region comprising the amino acid sequence of SEQ ID NO: 1145. In some embodiments, the anti-CD3 antibody comprises a constant region comprising the amino acid sequence of SEQ ID NO: 1145, and the anti-tumor antibody comprises a constant region comprising the amino acid sequence of SEQ ID NO: 1144.

In some embodiments, the anti-CD3 antibody, or antigen-binding fragment thereof, is operably linked to a constant region comprising the amino acid sequence of SEQ ID NO: 1144. In some embodiments, the anti-CD3 antibody, or antigen-binding fragment thereof, is operably linked to a constant region comprising the amino acid sequence of SEQ ID NO: 1145. In some embodiments, the anti-tumor antibody, or antigen-binding fragment thereof, is operably linked to a constant region comprising the amino acid sequence of SEQ ID NO: 1144. In some embodiments, the anti-tumor antibody, or antigen-binding fragment thereof, is operably linked to a constant region comprising the amino acid sequence of SEQ ID NO: 1145. In some embodiments, the anti-CD3 antibody, or antigen-binding fragment thereof, is operably linked to a constant region comprising the amino acid sequence of SEQ ID NO: 1144, and the anti-tumor antibody, or antigen-binding fragment thereof, is operably linked to a constant region comprising the amino acid sequence of SEQ ID NO: 1145. In some embodiments, the anti-CD3 antibody, or antigen-binding fragment thereof, is operably linked to a constant region comprising the amino acid sequence of SEQ ID NO: 1145, and the anti-tumor antibody, or antigen-binding fragment thereof, is operably linked to a constant region comprising the amino acid sequence of SEQ ID NO: 1144.

In some embodiments, the anti-tumor antibody is an anti-CD33 antibody. In some embodiments, the anti-CD33 antibody comprises the six CDRs of gemtuzumab. In some embodiments, the anti-CD33 antibody comprises the VH of gemtuzumab. In some embodiments, the anti-CD33 antibody comprises the VL of gemtuzumab. In some embodiments, the anti-CD33 antibody comprises the VH and the VL of gemtuzumab. In some embodiments, the heavy chain of the anti-CD33 antibody comprises the amino acid sequence of SEQ ID NO: 1150. In some embodiments, the light chain of the anti-CD33 antibody comprises the amino acid sequence of SEQ ID NO: 1151. In some embodiments, the heavy chain of the anti-CD33 antibody comprises the amino acid sequence of SEQ ID NO: 1150, and the light chain of the anti-CD33 antibody comprises the amino acid sequence of SEQ ID NO: 1151.

One aspect of the disclosure provides a recombinant polynucleotide encoding the anti-CD3 antibody, or antigen-binding fragment thereof, of the disclosure or an anti-CD3 bispecific antibody of the disclosure. Another aspect of the disclosure provides a recombinant polynucleotide encoding the anti-CD3 antibody, or antigen-binding fragment thereof, of the disclosure or an anti-CD3 multi-specific antibody of the disclosure. Another aspect of the disclosure provides vector, such as an expression vector, comprising the recombinant polynucleotide. In another aspect of the disclosure provides an isolated host cell that comprises the recombinant polynucleotide or the vector. An additional aspect of the disclosure provides methods of making the anti-CD3 antibody, or antigen-binding fragment thereof, of the disclosure or an anti-CD3 bispecific antibody of the disclosure. One aspect of the disclosure provides a composition comprising an anti-CD3 antibody, or antigen-binding fragment thereof, of the disclosure or an anti-CD3 bispecific antibody of the disclosure and a physiologically acceptable carrier.

A further aspect of the disclosure provides methods of treating or preventing cancer in a subject in need thereof by administering an anti-CD3 antibody, or antigen-binding fragment thereof, or bispecific or multi-specific antibody of the disclosure or a composition comprising the antibody or fragment or bispecific or multi-specific antibody to a subject in need thereof. One aspect of the disclosure provides a method of maintaining cancer remission in a subject by administering an anti-CD3 antibody, or antigen-binding fragment thereof, or bispecific or multi-specific antibody of the disclosure or a composition comprising the antibody or fragment or bispecific antibody to a subject in need thereof. An additional aspect of the disclosure provides a method of redirecting a T-cell to a tumor cell, wherein the method comprises contacting the T-cell with an anti-CD3 bispecific or multi-specific antibody, or antigen-binding fragment thereof, of the disclosure or a composition comprising the bispecific or multi-specific antibody or fragment. In some embodiments, bispecific or multi-specific antibody or fragment further comprises a tumor-targeting arm. A further aspect of the invention provides a method of treating cancer by administering an anti-CD3 bispecific or multi-specific antibody, or antigen-binding fragment thereof, of the disclosure or a composition comprising the antibody or fragment to a subject in need thereof, wherein the bispecific or multi-specific antibody or fragment further comprises a tumor-targeting arm.

Another aspect of the disclosure provides methods of treating or preventing an autoimmune disease or disorder in a subject in need thereof by administering an anti-CD3 antibody, or antigen-binding fragment thereof, or bispecific or multi-specific antibody of the disclosure or a composition comprising the antibody or fragment or bispecific or multi-specific antibody to a subject in need thereof.

### BRIEF DESCRIPTION OF THE FIGURES

FIG. 1A and FIG. 1B provide the results of the flow cytometric analysis of purified monoclonal antibodies. Briefly, flow cytometry was performed at concentration 1 μg/ml against Jurkat wild-type and knock-out cells and human Pan-T cells. Log geometric mean florescent intensity (gMFI) values were plotted for each hit comparing Jurkat wild-type and knock-out cells (FIG. 1A) and Jurkat wild-type and Pan-T cells (FIG. 1B).

FIG. 2 provides a schematic of the formation of a CD3-CD33 bispecific antibody.

FIG. 3A provides a schematic of a Killing Immune-Lysis Reaction (KTLR) HL60 T Cell Redirection assay protocol. FIG. 3B demonstrates the assessment of CD-3×CD33 bispecific antibodies for their ability to mediate interactions between effector and target cells leading to target-mediated cytotoxicity.

FIG. 4A and FIG. 4B provide the results of quantitative ELISA assays against the cytokines TL2 (FIG. 4A) and IFNγ (FIG. 4B).

FIGS. 5A-5D provide the results of Killing Immune-Lysis Reaction (KTLR) T Cell Redirection (TCR) assays for four human PBMC donors (Donor 1 (FIG. 5A), Donor 2 (FIG. 5B), Donor 3 (FIG. 5C), and Donor 0 (FIG. 5D)).

FIG. 6A provides the results of an ELISA employed to measure polyspecific/nonspecific antibody binding to ds DNA and ss DNA, cardiolipin, insulin, lipopolysaccharide, and Hemocyanin. FIG. 6B provides the results of an ELISA employed to measure polyspecific/nonspecific antibody binding Baculovirus Particles. FIG. 6C provides results of the retention time of the antibody measured on a HIC-butyl column with less hydrophobic species eluting earlier and more hydrophobic species eluting later.

### BRIEF DESCRIPTION OF THE SEQUENCES

TABLE 1A
Anti-CD3 Antibody Amino Acid Sequence Identifiers

| Antibody | LCDR1 | LCDR2 | LCDR3 | HCDR1 | HCDR2 | HCDR3 | VL | VH |
|---|---|---|---|---|---|---|---|---|
| ABL-CD3-1 | 1 | 114 | 227 | 345 | 458 | 574 | 688 | 915 |
| ABL-CD3-2 | 2 | 115 | 228 | 346 | 459 | 575 | 689 | 916 |
| ABL-CD3-3 | 3 | 116 | 229 | 347 | 460 | 576 | 690 | 917 |
| ABL-CD3-4 | 4 | 117 | 230 | 348 | 461 | 577 | 691 | 918 |
| ABL-CD3-5 | 5 | 118 | 231 | 349 | 462 | 578 | 692 | 919 |
| ABL-CD3-6 | 6 | 119 | 232 | 350 | 463 | 579 | 693 | 920 |
| ABL-CD3-7 | 7 | 120 | 233 | 351 | 464 | 580 | 694 | 921 |
| ABL-CD3-8 | 8 | 121 | 234 | 352 | 465 | 581 | 695 | 922 |
| ABL-CD3-9 | 9 | 122 | 235 | 353 | 466 | 582 | 696 | 923 |
| ABL-CD3-10 | 10 | 123 | 236 | 354 | 467 | 583 | 697 | 924 |
| ABL-CD3-11 | 11 | 124 | 237 | 355 | 468 | 584 | 698 | 925 |
| ABL-CD3-12 | 12 | 125 | 238 | 356 | 469 | 585 | 699 | 926 |
| ABL-CD3-13 | 13 | 126 | 239 | 357 | 470 | 586 | 700 | 927 |
| ABL-CD3-14 | 14 | 127 | 240 | 358 | 471 | 587 | 701 | 928 |
| ABL-CD3-15 | 15 | 128 | 241 | 359 | 472 | 588 | 702 | 929 |
| ABL-CD3-16 | 16 | 129 | 242 | 360 | 473 | 589 | 703 | 930 |
| ABL-CD3-17 | 17 | 130 | 243 | 361 | 474 | 590 | 704 | 931 |
| ABL-CD3-18 | 18 | 131 | 244 | 362 | 475 | 591 | 705 | 932 |
| ABL-CD3-19 | 19 | 132 | 245 | 363 | 476 | 592 | 706 | 933 |
| ABL-CD3-20 | 20 | 133 | 246 | 364 | 477 | 593 | 707 | 934 |
| ABL-CD3-21 | 21 | 134 | 247 | 365 | 478 | 594 | 708 | 935 |
| ABL-CD3-22 | 22 | 135 | 248 | 366 | 479 | 595 | 709 | 936 |
| ABL-CD3-23 | 23 | 136 | 249 | 367 | 480 | 596 | 710 | 937 |
| ABL-CD3-24 | 24 | 137 | 250 | 368 | 481 | 597 | 711 | 938 |
| ABL-CD3-25 | 25 | 138 | 251 | 369 | 482 | 598 | 712 | 939 |
| ABL-CD3-26 | 26 | 139 | 252 | 370 | 483 | 599 | 713 | 940 |
| ABL-CD3-27 | 27 | 140 | 253 | 371 | 484 | 600 | 714 | 941 |
| ABL-CD3-28 | 28 | 141 | 254 | 372 | 485 | 601 | 715 | 942 |
| ABL-CD3-29 | 29 | 142 | 255 | 373 | 486 | 602 | 716 | 943 |
| ABL-CD3-30 | 30 | 143 | 256 | 374 | 487 | 603 | 717 | 944 |
| ABL-CD3-31 | 31 | 144 | 257 | 375 | 488 | 604 | 718 | 945 |
| ABL-CD3-32 | 32 | 145 | 258 | 376 | 489 | 605 | 719 | 946 |
| ABL-CD3-33 | 33 | 146 | 259 | 377 | 490 | 606 | 720 | 947 |
| ABL-CD3-34 | 34 | 147 | 260 | 378 | 491 | 607 | 721 | 948 |
| ABL-CD3-35 | 35 | 148 | 261 | 379 | 492 | 608 | 722 | 949 |
| ABL-CD3-36 | 36 | 149 | 262 | 380 | 493 | 609 | 723 | 950 |
| ABL-CD3-37 | 37 | 150 | 263 | 381 | 494 | 610 | 724 | 951 |
| ABL-CD3-38 | 38 | 151 | 264 | 382 | 495 | 611 | 725 | 952 |
| ABL-CD3-39 | 39 | 152 | 265 | 383 | 496 | 612 | 726 | 953 |
| ABL-CD3-40 | 40 | 153 | 266 | 384 | 497 | 613 | 727 | 954 |
| ABL-CD3-41 | 41 | 154 | 267 | 385 | 498 | 614 | 728 | 955 |
| ABL-CD3-42 | 42 | 155 | 268 | 386 | 499 | 615 | 729 | 956 |
| ABL-CD3-43 | 43 | 156 | 269 | 387 | 500 | 616 | 730 | 957 |
| ABL-CD3-44 | 44 | 157 | 270 | 388 | 501 | 617 | 731 | 958 |
| ABL-CD3-45 | 45 | 158 | 271 | 389 | 502 | 618 | 732 | 959 |
| ABL-CD3-46 | 46 | 159 | 272 | 390 | 503 | 619 | 733 | 960 |
| ABL-CD3-47 | 47 | 160 | 273 | 391 | 504 | 620 | 734 | 961 |
| ABL-CD3-48 | 48 | 161 | 274 | 392 | 505 | 621 | 735 | 962 |
| ABL-CD3-49 | 49 | 162 | 275 | 393 | 506 | 622 | 736 | 963 |
| ABL-CD3-50 | 50 | 163 | 276 | 394 | 507 | 623 | 737 | 964 |
| ABL-CD3-51 | 51 | 164 | 277 | 395 | 508 | 624 | 738 | 965 |
| ABL-CD3-52 | 52 | 165 | 278 | 396 | 509 | 625 | 739 | 966 |
| ABL-CD3-53 | 53 | 166 | 279 | 397 | 510 | 626 | 740 | 967 |
| ABL-CD3-54 | 54 | 167 | 280 | 398 | 511 | 627 | 741 | 968 |
| ABL-CD3-55 | 55 | 168 | 281 | 399 | 512 | 628 | 742 | 969 |
| ABL-CD3-56 | 56 | 169 | 282 | 400 | 513 | 629 | 743 | 970 |
| ABL-CD3-57 | 57 | 170 | 283 | 401 | 514 | 630 | 744 | 971 |
| ABL-CD3-58 | 58 | 171 | 284 | 402 | 515 | 631 | 745 | 972 |
| ABL-CD3-59 | 59 | 172 | 285 | 403 | 516 | 632 | 746 | 973 |
| ABL-CD3-60 | 60 | 173 | 286 | 404 | 517 | 633 | 747 | 974 |
| ABL-CD3-61 | 61 | 174 | 287 | 405 | 518 | 634 | 748 | 975 |
| ABL-CD3-62 | 62 | 175 | 288 | 406 | 519 | 635 | 749 | 976 |
| ABL-CD3-63 | 63 | 176 | 289 | 407 | 520 | 636 | 750 | 977 |
| ABL-CD3-64 | 64 | 177 | 290 | 408 | 521 | 637 | 751 | 978 |
| ABL-CD3-65 | 65 | 178 | 291 | 409 | 522 | 638 | 752 | 979 |
| ABL-CD3-66 | 66 | 179 | 292 | 410 | 523 | 639 | 753 | 980 |
| ABL-CD3-67 | 67 | 180 | 293 | 411 | 524 | 640 | 754 | 981 |
| ABL-CD3-68 | 68 | 181 | 294 | 412 | 525 | 641 | 755 | 982 |
| ABL-CD3-69 | 69 | 182 | 295 | 413 | 526 | 642 | 756 | 983 |
| ABL-CD3-70 | 70 | 183 | 296 | 414 | 527 | 643 | 757 | 984 |
| ABL-CD3-71 | 71 | 184 | 297 | 415 | 528 | 644 | 758 | 985 |
| ABL-CD3-72 | 72 | 185 | 298 | 416 | 529 | 645 | 759 | 986 |
| ABL-CD3-73 | 73 | 186 | 299 | 417 | 530 | 646 | 760 | 987 |
| ABL-CD3-74 | 74 | 187 | 300 | 418 | 531 | 647 | 761 | 988 |
| ABL-CD3-75 | 75 | 188 | 301 | 419 | 532 | 648 | 762 | 989 |
| ABL-CD3-76 | 76 | 189 | 302 | 420 | 533 | 649 | 763 | 990 |
| ABL-CD3-77 | 77 | 190 | 303 | 421 | 534 | 650 | 764 | 991 |
| ABL-CD3-78 | 78 | 191 | 304 | 422 | 535 | 651 | 765 | 992 |
| ABL-CD3-79 | 79 | 192 | 305 | 423 | 536 | 652 | 766 | 993 |
| ABL-CD3-80 | 80 | 193 | 306 | 424 | 537 | 653 | 767 | 994 |
| ABL-CD3-81 | 81 | 194 | 307 | 425 | 538 | 654 | 768 | 995 |
| ABL-CD3-82 | 82 | 195 | 308 | 426 | 539 | 655 | 769 | 996 |
| ABL-CD3-83 | 83 | 196 | 309 | 427 | 540 | 656 | 770 | 997 |
| ABL-CD3-84 | 84 | 197 | 310 | 428 | 541 | 657 | 771 | 998 |
| ABL-CD3-85 | 85 | 198 | 311 | 429 | 542 | 658 | 772 | 999 |
| ABL-CD3-86 | 86 | 199 | 312 | 430 | 543 | 659 | 773 | 1000 |
| ABL-CD3-87 | 87 | 200 | 313 | 431 | 544 | 660 | 774 | 1001 |
| ABL-CD3-88 | 88 | 201 | 314 | 432 | 545 | 661 | 775 | 1002 |
| ABL-CD3-89 | 89 | 202 | 315 | 433 | 546 | 662 | 776 | 1003 |
| ABL-CD3-90 | 90 | 203 | 316 | 434 | 547 | 663 | 777 | 1004 |
| ABL-CD3-91 | 91 | 204 | 317 | 435 | 548 | 664 | 778 | 1005 |
| ABL-CD3-92 | 92 | 205 | 318 | 436 | 549 | 665 | 779 | 1006 |
| ABL-CD3-93 | 93 | 206 | 319 | 437 | 550 | 666 | 780 | 1007 |
| ABL-CD3-94 | 94 | 207 | 320 | 438 | 551 | 667 | 781 | 1008 |
| ABL-CD3-95 | 95 | 208 | 321 | 439 | 552 | 668 | 782 | 1009 |
| ABL-CD3-96 | 96 | 209 | 322 | 440 | 553 | 669 | 783 | 1010 |
| ABL-CD3-97 | 97 | 210 | 323 | 441 | 554 | 670 | 784 | 1011 |
| ABL-CD3-98 | 98 | 211 | 324 | 442 | 555 | 671 | 785 | 1012 |
| ABL-CD3-99 | 99 | 212 | 325 | 443 | 556 | 672 | 786 | 1013 |
| ABL-CD3-100 | 100 | 213 | 326 | 444 | 557 | 673 | 787 | 1014 |
| ABL-CD3-101 | 101 | 214 | 327 | 445 | 558 | 674 | 788 | 1015 |
| ABL-CD3-102 | 102 | 215 | 328 | 446 | 559 | 675 | 789 | 1016 |
| ABL-CD3-103 | 103 | 216 | 329 | 447 | 560 | 676 | 790 | 1017 |
| ABL-CD3-104 | 104 | 217 | 330 | 448 | 561 | 677 | 791 | 1018 |
| ABL-CD3-105 | 105 | 218 | 331 | 449 | 562 | 678 | 792 | 1019 |
| ABL-CD3-106 | 106 | 219 | 332 | 450 | 563 | 679 | 793 | 1020 |
| ABL-CD3-107 | 107 | 220 | 333 | 451 | 564 | 680 | 794 | 1021 |
| ABL-CD3-108 | 108 | 221 | 334 | 452 | 565 | 681 | 795 | 1022 |
| ABL-CD3-109 | 109 | 222 | 335 | 453 | 566 | 682 | 796 | 1023 |
| ABL-CD3-110 | 110 | 223 | 336 | 454 | 567 | 683 | 797 | 1024 |
| ABL-CD3-111 | 111 | 224 | 337 | 455 | 568 | 684 | 798 | 1025 |
| ABL-CD3-112 | 112 | 225 | 338 | 456 | 569 | 685 | 799 | 1026 |
| ABL-CD3-113 | 113 | 226 | 339 | 457 | 570 | 686 | 800 | 1027 |
| ABL-CD3-1A | 1 | 114 | 227 | 345 | 458 | 574 | 801 | 1028 |
| ABL-CD3-2A | 2 | 115 | 228 | 346 | 459 | 575 | 802 | 1029 |
| ABL-CD3-3A | 3 | 116 | 229 | 347 | 460 | 576 | 803 | 1030 |
| ABL-CD3-4A | 4 | 117 | 230 | 348 | 461 | 577 | 804 | 1031 |
| ABL-CD3-5A | 5 | 118 | 231 | 349 | 462 | 578 | 805 | 1032 |
| ABL-CD3-6A | 6 | 119 | 232 | 350 | 463 | 579 | 806 | 1033 |
| ABL-CD3-7A | 7 | 120 | 233 | 351 | 464 | 580 | 807 | 1034 |
| ABL-CD3-8A | 8 | 121 | 234 | 352 | 465 | 581 | 808 | 1035 |
| ABL-CD3-9A | 9 | 122 | 235 | 353 | 466 | 582 | 809 | 1036 |
| ABL-CD3-10A | 10 | 123 | 236 | 354 | 467 | 583 | 810 | 1037 |
| ABL-CD3-11A | 11 | 124 | 237 | 355 | 468 | 584 | 811 | 1038 |
| ABL-CD3-12A | 12 | 125 | 238 | 356 | 469 | 585 | 812 | 1039 |
| ABL-CD3-13A | 13 | 126 | 239 | 357 | 470 | 586 | 813 | 1040 |
| ABL-CD3-14A | 14 | 127 | 240 | 358 | 471 | 587 | 814 | 1041 |
| ABL-CD3-15A | 15 | 128 | 241 | 359 | 472 | 588 | 815 | 1042 |
| ABL-CD3-16A | 16 | 129 | 242 | 360 | 473 | 589 | 816 | 1043 |
| ABL-CD3-17A | 17 | 130 | 243 | 361 | 474 | 590 | 817 | 1044 |
| ABL-CD3-18A | 18 | 131 | 244 | 362 | 475 | 591 | 818 | 1045 |
| ABL-CD3-19A | 19 | 132 | 245 | 363 | 476 | 592 | 819 | 1046 |
| ABL-CD3-20A | 20 | 133 | 246 | 364 | 477 | 593 | 820 | 1047 |
| ABL-CD3-21A | 21 | 134 | 247 | 365 | 478 | 594 | 821 | 1048 |
| ABL-CD3-22A | 22 | 135 | 248 | 366 | 479 | 595 | 822 | 1049 |
| ABL-CD3-23A | 23 | 136 | 249 | 367 | 480 | 596 | 823 | 1050 |
| ABL-CD3-24A | 24 | 137 | 250 | 368 | 481 | 597 | 824 | 1051 |
| ABL-CD3-25A | 25 | 138 | 251 | 369 | 482 | 598 | 825 | 1052 |
| ABL-CD3-26A | 26 | 139 | 252 | 370 | 483 | 599 | 826 | 1053 |
| ABL-CD3-27A | 27 | 140 | 253 | 371 | 484 | 687 | 827 | 1054 |
| ABL-CD3-28A | 28 | 141 | 254 | 372 | 485 | 601 | 828 | 1055 |
| ABL-CD3-29A | 29 | 142 | 255 | 373 | 486 | 602 | 829 | 1056 |
| ABL-CD3-30A | 30 | 143 | 256 | 374 | 487 | 603 | 830 | 1057 |
| ABL-CD3-31A | 31 | 144 | 257 | 375 | 488 | 604 | 831 | 1058 |
| ABL-CD3-32A | 32 | 145 | 258 | 376 | 489 | 605 | 832 | 1059 |
| ABL-CD3-33A | 33 | 146 | 259 | 377 | 490 | 606 | 833 | 1060 |
| ABL-CD3-34A | 34 | 147 | 260 | 378 | 491 | 607 | 834 | 1061 |
| ABL-CD3-35A | 35 | 148 | 261 | 379 | 492 | 608 | 835 | 1062 |
| ABL-CD3-36A | 36 | 149 | 262 | 380 | 571 | 609 | 836 | 1063 |
| ABL-CD3-37A | 37 | 150 | 263 | 381 | 494 | 610 | 837 | 1064 |
| ABL-CD3-38A | 38 | 151 | 264 | 382 | 495 | 611 | 838 | 1065 |
| ABL-CD3-39A | 39 | 152 | 265 | 383 | 496 | 612 | 839 | 1066 |
| ABL-CD3-40A | 40 | 153 | 266 | 384 | 497 | 613 | 840 | 1067 |
| ABL-CD3-41A | 41 | 154 | 340 | 385 | 498 | 614 | 841 | 1068 |
| ABL-CD3-42A | 42 | 155 | 268 | 386 | 499 | 615 | 842 | 1069 |
| ABL-CD3-43A | 43 | 156 | 341 | 387 | 500 | 616 | 843 | 1070 |
| ABL-CD3-44A | 44 | 157 | 270 | 388 | 501 | 617 | 844 | 1071 |
| ABL-CD3-45A | 45 | 158 | 271 | 389 | 502 | 618 | 845 | 1072 |
| ABL-CD3-46A | 46 | 159 | 272 | 390 | 503 | 619 | 846 | 1073 |
| ABL-CD3-47A | 47 | 160 | 273 | 391 | 504 | 620 | 847 | 1074 |
| ABL-CD3-48A | 48 | 161 | 274 | 392 | 505 | 621 | 848 | 1075 |
| ABL-CD3-49A | 49 | 162 | 342 | 393 | 506 | 622 | 849 | 1076 |
| ABL-CD3-50A | 50 | 163 | 276 | 394 | 507 | 623 | 850 | 1077 |
| ABL-CD3-51A | 51 | 164 | 277 | 395 | 508 | 624 | 851 | 1078 |
| ABL-CD3-52A | 52 | 165 | 278 | 396 | 509 | 625 | 852 | 1079 |
| ABL-CD3-53A | 53 | 166 | 279 | 397 | 510 | 626 | 853 | 1080 |
| ABL-CD3-54A | 54 | 167 | 280 | 398 | 511 | 627 | 854 | 1081 |
| ABL-CD3-55A | 55 | 168 | 281 | 399 | 512 | 628 | 855 | 1082 |
| ABL-CD3-56A | 56 | 169 | 282 | 400 | 513 | 629 | 856 | 1083 |
| ABL-CD3-57A | 57 | 170 | 283 | 401 | 514 | 630 | 857 | 1084 |
| ABL-CD3-58A | 58 | 171 | 284 | 402 | 515 | 631 | 858 | 1085 |
| ABL-CD3-59A | 59 | 172 | 285 | 403 | 516 | 632 | 859 | 1086 |
| ABL-CD3-60A | 60 | 173 | 286 | 404 | 517 | 633 | 860 | 1087 |
| ABL-CD3-61A | 61 | 174 | 287 | 405 | 518 | 634 | 861 | 1088 |
| ABL-CD3-62A | 62 | 175 | 288 | 406 | 519 | 635 | 862 | 1089 |
| ABL-CD3-63A | 63 | 176 | 289 | 407 | 520 | 636 | 863 | 1090 |
| ABL-CD3-64A | 64 | 177 | 290 | 408 | 521 | 637 | 864 | 1091 |
| ABL-CD3-65A | 65 | 178 | 291 | 409 | 572 | 638 | 865 | 1092 |
| ABL-CD3-66A | 66 | 179 | 292 | 410 | 523 | 639 | 866 | 1093 |
| ABL-CD3-67A | 67 | 180 | 293 | 411 | 524 | 640 | 867 | 1094 |
| ABL-CD3-68A | 68 | 181 | 294 | 412 | 525 | 641 | 868 | 1095 |
| ABL-CD3-69A | 69 | 182 | 295 | 413 | 526 | 642 | 869 | 1096 |
| ABL-CD3-70A | 70 | 183 | 296 | 414 | 527 | 643 | 870 | 1097 |
| ABL-CD3-71A | 71 | 184 | 297 | 415 | 528 | 644 | 871 | 1098 |
| ABL-CD3-72A | 72 | 185 | 298 | 416 | 529 | 645 | 872 | 1099 |
| ABL-CD3-73A | 73 | 186 | 299 | 417 | 530 | 646 | 873 | 1100 |
| ABL-CD3-74A | 74 | 187 | 300 | 418 | 531 | 647 | 874 | 1101 |
| ABL-CD3-75A | 75 | 188 | 301 | 419 | 532 | 648 | 875 | 1102 |
| ABL-CD3-76A | 76 | 189 | 302 | 420 | 533 | 649 | 876 | 1103 |
| ABL-CD3-77A | 77 | 190 | 303 | 421 | 534 | 650 | 877 | 1104 |
| ABL-CD3-78A | 78 | 191 | 304 | 422 | 535 | 651 | 878 | 1105 |
| ABL-CD3-79A | 79 | 192 | 305 | 423 | 573 | 652 | 879 | 1106 |
| ABL-CD3-80A | 80 | 193 | 306 | 424 | 537 | 653 | 880 | 1107 |
| ABL-CD3-81A | 81 | 194 | 307 | 425 | 538 | 654 | 881 | 1108 |
| ABL-CD3-82A | 82 | 195 | 308 | 426 | 539 | 655 | 882 | 1109 |
| ABL-CD3-83A | 83 | 196 | 309 | 427 | 540 | 656 | 883 | 1110 |
| ABL-CD3-84A | 84 | 197 | 310 | 428 | 541 | 657 | 884 | 1111 |
| ABL-CD3-85A | 85 | 198 | 343 | 429 | 542 | 658 | 885 | 1112 |
| ABL-CD3-85B | 85 | 198 | 344 | 429 | 542 | 658 | 886 | 1113 |
| ABL-CD3-86A | 86 | 199 | 312 | 430 | 543 | 659 | 887 | 1114 |
| ABL-CD3-87A | 87 | 200 | 313 | 431 | 544 | 660 | 888 | 1115 |
| ABL-CD3-88A | 88 | 201 | 314 | 432 | 545 | 661 | 889 | 1116 |
| ABL-CD3-89A | 89 | 202 | 315 | 433 | 546 | 662 | 890 | 1117 |
| ABL-CD3-90A | 90 | 203 | 316 | 434 | 547 | 663 | 891 | 1118 |
| ABL-CD3-91A | 91 | 204 | 317 | 435 | 548 | 664 | 892 | 1119 |
| ABL-CD3-92A | 92 | 205 | 318 | 436 | 549 | 665 | 893 | 1120 |
| ABL-CD3-93A | 93 | 206 | 319 | 437 | 550 | 666 | 894 | 1121 |
| ABL-CD3-94A | 94 | 207 | 320 | 438 | 551 | 667 | 895 | 1122 |
| ABL-CD3-95A | 95 | 208 | 321 | 439 | 552 | 668 | 896 | 1123 |
| ABL-CD3-96A | 96 | 209 | 322 | 440 | 553 | 669 | 897 | 1124 |
| ABL-CD3-97A | 97 | 210 | 323 | 441 | 554 | 670 | 898 | 1125 |
| ABL-CD3-98A | 98 | 211 | 324 | 442 | 555 | 671 | 899 | 1126 |
| ABL-CD3-99A | 99 | 212 | 325 | 443 | 556 | 672 | 900 | 1127 |
| ABL-CD3-100A | 100 | 213 | 326 | 444 | 557 | 673 | 901 | 1128 |
| ABL-CD3-101A | 101 | 214 | 327 | 445 | 558 | 674 | 902 | 1129 |
| ABL-CD3-102A | 102 | 215 | 328 | 446 | 559 | 675 | 903 | 1130 |
| ABL-CD3-103A | 103 | 216 | 329 | 447 | 560 | 676 | 904 | 1131 |
| ABL-CD3-104A | 104 | 217 | 330 | 448 | 561 | 677 | 905 | 1132 |
| ABL-CD3-105A | 105 | 218 | 331 | 449 | 562 | 678 | 906 | 1133 |
| ABL-CD3-106A | 106 | 219 | 332 | 450 | 563 | 679 | 907 | 1134 |
| ABL-CD3-107A | 107 | 220 | 333 | 451 | 564 | 680 | 908 | 1135 |
| ABL-CD3-108A | 108 | 221 | 334 | 452 | 565 | 681 | 909 | 1136 |
| ABL-CD3-109A | 109 | 222 | 335 | 453 | 566 | 682 | 910 | 1137 |
| ABL-CD3-110A | 110 | 223 | 336 | 454 | 567 | 683 | 911 | 1138 |
| ABL-CD3-111A | 111 | 224 | 337 | 455 | 568 | 684 | 912 | 1139 |
| ABL-CD3-112A | 112 | 225 | 338 | 456 | 569 | 685 | 913 | 1140 |
| ABL-CD3-113A | 113 | 226 | 339 | 457 | 570 | 686 | 914 | 1141 |

TABLE 1B
LCDR Sequences

| | | |
|---|---|---|
| SEQ ID NO: | Antibody | Sequence |

Light Chain CDR1 Sequences

| | | |
|---|---|---|
| 1 | ABL-CD3-1/ABL-CD3-1A | TRSSGSIASNYVQ |
| 2 | ABL-CD3-2/ABL-CD3-2A | SGDKLGNKYAC |
| 3 | ABL-CD3-3/ABL-CD3-3A | SGDKLRDKYAC |
| 4 | ABL-CD3-4/ABL-CD3-4A | SGDKLGDKYAG |
| 5 | ABL-CD3-5/ABL-CD3-5A | TRSSGSIASNYVQ |
| 6 | ABL-CD3-6/ABL-CD3-6A | SGDALPKKYAY |
| 7 | ABL-CD3-7/ABL-CD3-7A | TRSSGSIASNYVQ |
| 8 | ABL-CD3-8/ABL-CD3-8A | SGDALPKKYAY |
| 9 | ABL-CD3-9/ABL-CD3-9A | SGDALPKKYAY |
| 10 | ABL-CD3-10/ABL-CD3-10A | TRSSGSIASNYVQ |
| 11 | ABL-CD3-11/ABL-CD3-11A | TRSSGSIASNYVQ |
| 12 | ABL-CD3-12/ABL-CD3-12A | TRSNGSITSNYVQ |
| 13 | ABL-CD3-13/ABL-CD3-13A | RASQSISNWLA |
| 14 | ABL-CD3-14/ABL-CD3-14A | TRSSGSIASNYVQ |
| 15 | ABL-CD3-15/ABL-CD3-15A | TRSSGSIASNYVQ |
| 16 | ABL-CD3-16/ABL-CD3-16A | RASQSIGSWLA |
| 17 | ABL-CD3-17/ABL-CD3-17A | TRSSGSIASKYVQ |
| 18 | ABL-CD3-18/ABL-CD3-18A | RSSQSLVHSDGNTYLN |
| 19 | ABL-CD3-19/ABL-CD3-19A | TRSSGSIASNYVQ |
| 20 | ABL-CD3-20/ABL-CD3-20A | TRSSGSIASNYVQ |
| 21 | ABL-CD3-21/ ABL-CD3-21A | SGDALPKKYAY |
| 22 | ABL-CD3-22/ABL-CD3-22A | SGDALPKQYTY |
| 23 | ABL-CD3-23/ABL-CD3-23A | TRSSGSIASNYVQ |
| 24 | ABL-CD3-24/ABL-CD3-24A | TRSSGSIASNYVQ |
| 25 | ABL-CD3-25/ABL-CD3-25A | TRSSGSIASNYVQ |
| 26 | ABL-CD3-26/ABL-CD3-26A | TRSSGSIASNYVQ |
| 27 | ABL-CD3-27/ABL-CD3-27A | GLNSGSVSTSYYPS |
| 28 | ABL-CD3-28/ABL-CD3-28A | TRSSGSIASNYVQ |
| 29 | ABL-CD3-29/ABL-CD3-29A | SGSSSNIGSNYVY |
| 30 | ABL-CD3-30/ABL-CD3-30A | TRSSGSIASNYVQ |
| 31 | ABL-CD3-31/ABL-CD3-31A | RSSQSLLHSNGYNYLD |
| 32 | ABL-CD3-32/ABL-CD3-32A | SGDKLGYKYAS |
| 33 | ABL-CD3-33/ABL-CD3-33A | TRSSGSIASNYVQ |
| 34 | ABL-CD3-34/ABL-CD3-34A | TRSSGSIASNYVQ |
| 35 | ABL-CD3-35/ABL-CD3-35A | SGDALPKKYAY |
| 36 | ABL-CD3-36/ABL-CD3-36A | RASQSISSWLA |
| 37 | ABL-CD3-37/ABL-CD3-37A | RASQSISSWLA |
| 38 | ABL-CD3-38/ABL-CD3-38A | TGSSSNIGAGYDVH |
| 39 | ABL-CD3-39/ABL-CD3-39A | RSSQSLVYSDGNTYLT |
| 40 | ABL-CD3-40/ABL-CD3-40A | SGSSSNIGSNTVN |
| 41 | ABL-CD3-41/ABL-CD3-41A | SGDALPKKYAY |
| 42 | ABL-CD3-42/ABL-CD3-42A | SGDALPKQYTY |
| 43 | ABL-CD3-43/ABL-CD3-43A | TGTSSDVGSYNLVS |
| 44 | ABL-CD3-44/ABL-CD3-44A | MLSSGFSVGDFWIR |
| 45 | ABL-CD3-45/ABL-CD3-45A | RASQSISSWLA |
| 46 | ABL-CD3-46/ABL-CD3-46A | TRSSGSIASNYVQ |
| 47 | ABL-CD3-47/ABL-CD3-47A | TRSSGSIASKYVQ |
| 48 | ABL-CD3-48/ABL-CD3-48A | TRSSGSIASNYVQ |
| 49 | ABL-CD3-49/ABL-CD3-49A | TRSSGSIASNYVQ |
| 50 | ABL-CD3-50/ABL-CD3-50A | TRSSGSIASNYVQ |
| 51 | ABL-CD3-51/ABL-CD3-51A | TRSSGSIASNYVQ |
| 52 | ABL-CD3-52/ABL-CD3-52A | SGDALPKKYAY |
| 53 | ABL-CD3-53/ABL-CD3-53A | TRSSGSIASNYVQ |
| 54 | ABL-CD3-54/ABL-CD3-54A | TRSSGSIASYYVQ |
| 55 | ABL-CD3-55/ABL-CD3-55A | TRSSGSIASNYVQ |
| 56 | ABL-CD3-56/ABL-CD3-56A | TRSSGSIASNYVQ |
| 57 | ABL-CD3-57/ABL-CD3-57A | MLSSGFSVGDFWIR |
| 58 | ABL-CD3-58/ABL-CD3-58A | TRSSGSIASNFVQ |
| 59 | ABL-CD3-59/ABL-CD3-59A | TRSSGSIASNYVQ |
| 60 | ABL-CD3-60/ABL-CD3-60A | TRSSGSIASNYVQ |
| 61 | ABL-CD3-61/ABL-CD3-61A | ASSTGAVTSGYYPN |
| 62 | ABL-CD3-62/ABL-CD3-62A | TRSSGSIASNYVQ |
| 63 | ABL-CD3-63/ABL-CD3-63A | TRTSGSIASNYVQ |
| 64 | ABL-CD3-64/ABL-CD3-64A | TRSSGSIASNYVQ |
| 65 | ABL-CD3-65/ABL-CD3-65A | SGSSSNIGSNYVY |
| 66 | ABL-CD3-66/ABL-CD3-66A | TRSSGSIASNYVQ |
| 67 | ABL-CD3-67/ABL-CD3-67A | TRSSGSIASNYVQ |
| 68 | ABL-CD3-68/ABL-CD3-68A | SGDALPKQYAY |
| 69 | ABL-CD3-69/ABL-CD3-69A | TRSSGSIASNYVQ |
| 70 | ABL-CD3-70/ABL-CD3-70A | TRSSGSIASNYVQ |
| 71 | ABL-CD3-71/ABL-CD3-71A | TRSSGSIASNYVQ |
| 72 | ABL-CD3-72/ABL-CD3-72A | TRSSGSIASNYVQ |
| 73 | ABL-CD3-73/ABL-CD3-73A | TRSSGSIASNYVQ |
| 74 | ABL-CD3-74/ABL-CD3-74A | SGDALPKKYAY |
| 75 | ABL-CD3-75/ABL-CD3-75A | SGDALPKKYAY |
| 76 | ABL-CD3-76/ABL-CD3-76A | SGDALPKKYAY |
| 77 | ABL-CD3-77/ABL-CD3-77A | TGSSSNIGAGYDVH |
| 78 | ABL-CD3-78/ABL-CD3-78A | TRSSGSIASNYVQ |
| 79 | ABL-CD3-79/ABL-CD3-79A | GLSSGSVSTSYYPS |
| 80 | ABL-CD3-80/ABL-CD3-80A | SGDALPKKYAY |
| 81 | ABL-CD3-81/ABL-CD3-81A | TRSSGSIASNYVQ |
| 82 | ABL-CD3-82/ABL-CD3-82A | SGDALPKKYAY |
| 83 | ABL-CD3-83/ABL-CD3-83A | TRSSGSIASNYVQ |
| 84 | ABL-CD3-84/ABL-CD3-84A | TRSSGSIASNYVQ |
| 85 | ABL-CD3-85/ABL-CD3-85A | TRSSGSIASNYVQ |
| 86 | ABL-CD3-86/ABL-CD3-86A | TRNSGSIASNYVQ |
| 87 | ABL-CD3-87/ABL-CD3-87A | TRSSGSIASNYVQ |
| 88 | ABL-CD3-88/ABL-CD3-88A | RASQSISSWLA |
| 89 | ABL-CD3-89/ABL-CD3-89A | RASQSVSSSYLA |
| 90 | ABL-CD3-90/ABL-CD3-90A | TRSSGSIASNYVQ |
| 91 | ABL-CD3-91/ABL-CD3-91A | TRSSGSIASNYVQ |
| 92 | ABL-CD3-92/ABL-CD3-92A | SGDKLGNKYAC |
| 93 | ABL-CD3-93/ABL-CD3-93A | TRSSGSIASNYVQ |
| 94 | ABL-CD3-94/ABL-CD3-94A | TRSSGSIASNYVQ |
| 95 | ABL-CD3-95/ABL-CD3-95A | TRSSGSIASNYVQ |
| 96 | ABL-CD3-96/ABL-CD3-96A | TRSSGSIASNYVQ |
| 97 | ABL-CD3-97/ABL-CD3-97A | SGDKLGDKYAC |
| 98 | ABL-CD3-98/ABL-CD3-98A | TRSSGSIASNYVQ |
| 99 | ABL-CD3-99/ABL-CD3-99A | TRSSGSIASNYVQ |
| 100 | ABL-CD3-100/ABL-CD3-100A | SGDKLGNKYAC |
| 101 | ABL-CD3-101/ABL-CD3-101A | TRSSGSIASNYVQ |
| 102 | ABL-CD3-102/ABL-CD3-102A | TRSSGSIASNYVQ |
| 103 | ABL-CD3-103/ABL-CD3-103A | TRSSGSIASNYVQ |
| 104 | ABL-CD3-104/ABL-CD3-104A | TRSSGSIASNYVQ |
| 105 | ABL-CD3-105/ABL-CD3-105A | TRSSGSIASNYVQ |
| 106 | ABL-CD3-106/ABL-CD3-106A | TRSSGSIASNYVQ |
| 107 | ABL-CD3-107/ABL-CD3-107A | SGSSSNIGSNTVN |
| 108 | ABL-CD3-108/ABL-CD3-108A | TRSSGSIASNYVQ |
| 109 | ABL-CD3-109/ABL-CD3-109A | TRSSGSIASNYVQ |
| 110 | ABL-CD3-110/ABL-CD3-110A | TRSSGSIASNYVQ |
| 111 | ABL-CD3-111/ABL-CD3-111A | RASQSVSSSYLA |
| 112 | ABL-CD3-112/ABL-CD3-112A | GLSSGSVSTSYYPS |
| 113 | ABL-CD3-113/ABL-CD3-113A | GLSSGSVSTSYYPS |

Light Chain CDR2 Sequences

| | | |
|---|---|---|
| 114 | ABL-CD3-1/ABL-CD3-1A | EDNQRPS |
| 115 | ABL-CD3-2/ABL-CD3-2A | QDSKRPS |
| 116 | ABL-CD3-3/ABL-CD3-3A | QDSKRPS |
| 117 | ABL-CD3-4/ABL-CD3-4A | QDSKRPS |
| 118 | ABL-CD3-5/ABL-CD3-5A | EDNQRPS |
| 119 | ABL-CD3-6/ABL-CD3-6A | EDSKRPS |
| 120 | ABL-CD3-7/ABL-CD3-7A | EDNQRPS |
| 121 | ABL-CD3-8/ABL-CD3-8A | EDSKRPS |
| 122 | ABL-CD3-9/ABL-CD3-9A | EDSKRPS |
| 123 | ABL-CD3-10/ABL-CD3-10A | EDNQRPS |
| 124 | ABL-CD3-11/ABL-CD3-11A | EDNQRPS |
| 125 | ABL-CD3-12/ABL-CD3-12A | EDNQRPS |
| 126 | ABL-CD3-13/ABL-CD3-13A | KASSLES |
| 127 | ABL-CD3-14/ABL-CD3-14A | EDNQRPS |
| 128 | ABL-CD3-15/ABL-CD3-15A | EDNQRPS |
| 129 | ABL-CD3-16/ABL-CD3-16A | KASSLES |
| 130 | ABL-CD3-17/ABL-CD3-17A | EDNQRPS |
| 131 | ABL-CD3-18/ABL-CD3-18A | KISDRFS |
| 132 | ABL-CD3-19/ABL-CD3-19A | EDNQRPS |
| 133 | ABL-CD3-20/ABL-CD3-20A | EDNQRPS |
| 134 | ABL-CD3-21/ ABL-CD3-21A | EDSKRPS |
| 135 | ABL-CD3-22/ABL-CD3-22A | KDSERPS |
| 136 | ABL-CD3-23/ABL-CD3-23A | EDNHRPS |
| 137 | ABL-CD3-24/ABL-CD3-24A | EDNQRPS |
| 138 | ABL-CD3-25/ABL-CD3-25A | EDNHRPS |
| 139 | ABL-CD3-26/ABL-CD3-26A | EDNHRPS |
| 140 | ABL-CD3-27/ABL-CD3-27A | STNTRSS |
| 141 | ABL-CD3-28/ABL-CD3-28A | EDNQRPS |
| 142 | ABL-CD3-29/ABL-CD3-29A | SNNQRPS |
| 143 | ABL-CD3-30/ABL-CD3-30A | EDDQRPS |
| 144 | ABL-CD3-31/ABL-CD3-31A | LGSNRAS |
| 145 | ABL-CD3-32/ABL-CD3-32A | QDSKRPS |
| 146 | ABL-CD3-33/ABL-CD3-33A | EDNQRPS |
| 147 | ABL-CD3-34/ABL-CD3-34A | EDNQRPS |
| 148 | ABL-CD3-35/ABL-CD3-35A | EDSKRPS |
| 149 | ABL-CD3-36/ABL-CD3-36A | KASSLES |
| 150 | ABL-CD3-37/ABL-CD3-37A | KASSLES |
| 151 | ABL-CD3-38/ABL-CD3-38A | GNSNRPS |
| 152 | ABL-CD3-39/ABL-CD3-39A | KVSNRDS |
| 153 | ABL-CD3-40/ABL-CD3-40A | SNNQRPS |
| 154 | ABL-CD3-41/ABL-CD3-41A | EDSKRPS |
| 155 | ABL-CD3-42/ABL-CD3-42A | KDSERPS |
| 156 | ABL-CD3-43/ABL-CD3-43A | EDIRRPS |
| 157 | ABL-CD3-44/ABL-CD3-44A | YHSDSNK |
| 158 | ABL-CD3-45/ABL-CD3-45A | KASSLES |
| 159 | ABL-CD3-46/ABL-CD3-46A | EDNQRPS |
| 160 | ABL-CD3-47/ABL-CD3-47A | EDNQRPS |
| 161 | ABL-CD3-48/ABL-CD3-48A | EDNQRPS |
| 162 | ABL-CD3-49/ABL-CD3-49A | EDNQRSS |
| 163 | ABL-CD3-50/ABL-CD3-50A | EDNQRPS |
| 164 | ABL-CD3-51/ABL-CD3-51A | EDNQRPS |
| 165 | ABL-CD3-52/ABL-CD3-52A | EDSKRPS |
| 166 | ABL-CD3-53/ABL-CD3-53A | EDNQRPS |
| 167 | ABL-CD3-54/ABL-CD3-54A | EDNQRPS |
| 168 | ABL-CD3-55/ABL-CD3-55A | EDNQRPS |
| 169 | ABL-CD3-56/ABL-CD3-56A | EDNQRPS |
| 170 | ABL-CD3-57/ABL-CD3-57A | YHSDSNK |
| 171 | ABL-CD3-58/ABL-CD3-58A | EDNQRPS |
| 172 | ABL-CD3-59/ABL-CD3-59A | EDNQRPS |
| 173 | ABL-CD3-60/ABL-CD3-60A | EDNQRPS |
| 174 | ABL-CD3-61/ABL-CD3-61A | STSNKHS |
| 175 | ABL-CD3-62/ABL-CD3-62A | EDNQRPS |
| 176 | ABL-CD3-63/ABL-CD3-63A | EDNQRPS |
| 177 | ABL-CD3-64/ABL-CD3-64A | EDNQRPS |
| 178 | ABL-CD3-65/ABL-CD3-65A | SNNQRPS |
| 179 | ABL-CD3-66/ABL-CD3-66A | EDNQRPS |
| 180 | ABL-CD3-67/ABL-CD3-67A | EDNQRPS |
| 181 | ABL-CD3-68/ABL-CD3-68A | KDSERPS |
| 182 | ABL-CD3-69/ABL-CD3-69A | EDNQRPS |
| 183 | ABL-CD3-70/ABL-CD3-70A | EDNQRPS |
| 184 | ABL-CD3-71/ABL-CD3-71A | EDNQRPS |
| 185 | ABL-CD3-72/ABL-CD3-72A | EDNQRPS |
| 186 | ABL-CD3-73/ABL-CD3-73A | EDNRRPS |
| 187 | ABL-CD3-74/ABL-CD3-74A | EDSKRPS |
| 188 | ABL-CD3-75/ABL-CD3-75A | EDSKRPS |
| 189 | ABL-CD3-76/ABL-CD3-76A | EDSKRPS |
| 190 | ABL-CD3-77/ABL-CD3-77A | GNSNRPS |
| 191 | ABL-CD3-78/ABL-CD3-78A | EDNQRPS |
| 192 | ABL-CD3-79/ABL-CD3-79A | STNTRSS |
| 193 | ABL-CD3-80/ABL-CD3-80A | EDSKRPS |
| 194 | ABL-CD3-81/ABL-CD3-81A | EDNQRPS |
| 195 | ABL-CD3-82/ABL-CD3-82A | EDSKRPS |
| 196 | ABL-CD3-83/ABL-CD3-83A | EDNQRPS |
| 197 | ABL-CD3-84/ABL-CD3-84A | EDTQRPS |
| 198 | ABL-CD3-85/ABL-CD3-85A | EDNHRPS |
| 199 | ABL-CD3-86/ABL-CD3-86A | EDKORPS |
| 200 | ABL-CD3-87/ABL-CD3-87A | EDNQRPS |
| 201 | ABL-CD3-88/ABL-CD3-88A | KASSLES |
| 202 | ABL-CD3-89/ABL-CD3-89A | GASSRAT |
| 203 | ABL-CD3-90/ABL-CD3-90A | EDNQRPS |
| 204 | ABL-CD3-91/ABL-CD3-91A | EDNQRPS |
| 205 | ABL-CD3-92/ABL-CD3-92A | QDNKRPS |
| 206 | ABL-CD3-93/ABL-CD3-93A | EDNQRPS |
| 207 | ABL-CD3-94/ABL-CD3-94A | EDNQRPS |
| 208 | ABL-CD3-95/ABL-CD3-95A | EDNQRPS |
| 209 | ABL-CD3-96/ABL-CD3-96A | EDNQRPS |
| 210 | ABL-CD3-97/ABL-CD3-97A | QDSKRPS |
| 211 | ABL-CD3-98/ABL-CD3-98A | EDNQRPS |
| 212 | ABL-CD3-99/ABL-CD3-99A | EDNQRPS |
| 213 | ABL-CD3-100/ABL-CD3-100A | QDSKRPS |
| 214 | ABL-CD3-101/ABL-CD3-101A | EDNQRPS |
| 215 | ABL-CD3-102/ABL-CD3-102A | EDNQRPS |
| 216 | ABL-CD3-103/ABL-CD3-103A | EDNQRPS |
| 217 | ABL-CD3-104/ABL-CD3-104A | EDNQRPS |
| 218 | ABL-CD3-105/ABL-CD3-105A | EDNQRPS |
| 219 | ABL-CD3-106/ABL-CD3-106A | EDNQRPS |
| 220 | ABL-CD3-107/ABL-CD3-107A | SNNQRPS |
| 221 | ABL-CD3-108/ABL-CD3-108A | EDNQRPS |
| 222 | ABL-CD3-109/ABL-CD3-109A | EDNQRPS |
| 223 | ABL-CD3-110/ABL-CD3-110A | EDNQRPS |
| 224 | ABL-CD3-111/ABL-CD3-111A | GASSRAT |
| 225 | ABL-CD3-112/ABL-CD3-112A | STNTHSS |
| 226 | ABL-CD3-113/ABL-CD3-113A | STNTHSS |

Light Chain CDR3 Sequences

| | | |
|---|---|---|
| 227 | ABL-CD3-1/ABL-CD3-1A | QSYDSSNRV |
| 228 | ABL-CD3-2/ABL-CD3-2A | QAWDISTVV |
| 229 | ABL-CD3-3/ABL-CD3-3A | QAWDSSTVV |
| 230 | ABL-CD3-4/ABL-CD3-4A | QAWDSSTVV |
| 231 | ABL-CD3-5/ABL-CD3-5A | QSYDSSNHV |
| 232 | ABL-CD3-6/ABL-CD3-6A | YSTDSSGNHSWV |
| 233 | ABL-CD3-7/ABL-CD3-7A | QSFDNSNRV |
| 234 | ABL-CD3-8/ABL-CD3-8A | YSTDSSGNHSWV |
| 235 | ABL-CD3-9/ABL-CD3-9A | YSTDSSGNHCWV |
| 236 | ABL-CD3-10/ABL-CD3-10A | QSLDSSSVV |
| 237 | ABL-CD3-11/ABL-CD3-11A | QSYDSSNQV |
| 238 | ABL-CD3-12/ABL-CD3-12A | QSFDNSNRV |
| 239 | ABL-CD3-13/ABL-CD3-13A | QQYKSYSWT |
| 240 | ABL-CD3-14/ABL-CD3-14A | QSYDSSNRV |
| 241 | ABL-CD3-15/ABL-CD3-15A | QSYDSSNRV |
| 242 | ABL-CD3-16/ABL-CD3-16A | QQYNSYSWT |
| 243 | ABL-CD3-17/ABL-CD3-17A | QSYDSNNRV |
| 244 | ABL-CD3-18/ABL-CD3-18A | MQSTQFPIT |
| 245 | ABL-CD3-19/ABL-CD3-19A | QSFDNSNRV |
| 246 | ABL-CD3-20/ABL-CD3-20A | QSYDRSNRV |
| 247 | ABL-CD3-21/ ABL-CD3-21A | YSTHSSGNHR V |
| 248 | ABL-CD3-22/ABL-CD3-22A | QSADSSGTYRV |
| 249 | ABL-CD3-23/ABL-CD3-23A | QSFDSSNRV |
| 250 | ABL-CD3-24/ABL-CD3-24A | QSFYSSNRV |
| 251 | ABL-CD3-25/ABL-CD3-25A | QSFDSSNRV |
| 252 | ABL-CD3-26/ABL-CD3-26A | QSFDSSNRV |
| 253 | ABL-CD3-27/ABL-CD3-27A | VLYMGSGIWV |
| 254 | ABL-CD3-28/ABL-CD3-28A | QSFDSSNVV |
| 255 | ABL-CD3-29/ABL-CD3-29A | EAWDDSLSGPV |
| 256 | ABL-CD3-30/ABL-CD3-30A | QSYNSSNQV |
| 257 | ABL-CD3-31/ABL-CD3-31A | MQPLQTPYT |
| 258 | ABL-CD3-32/ABL-CD3-32A | QAWDSSTVV |
| 259 | ABL-CD3-33/ABL-CD3-33A | QSYYNSNRV |
| 260 | ABL-CD3-34/ABL-CD3-34A | QSYDSSNRV |
| 261 | ABL-CD3-35/ABL-CD3-35A | YSTDSSGNHCWV |
| 262 | ABL-CD3-36/ABL-CD3-36A | QQYISYSWT |
| 263 | ABL-CD3-37/ABL-CD3-37A | QQYNSYWT |
| 264 | ABL-CD3-38/ABL-CD3-38A | QSYDSSLSGYV |
| 265 | ABL-CD3-39/ABL-CD3-39A | MQGTHWPLT |
| 266 | ABL-CD3-40/ABL-CD3-40A | APWDDSLNGVV |
| 267 | ABL-CD3-41 | YSTDSSGNHCWV |
| 268 | ABL-CD3-42/ABL-CD3-42A | QSADSSGTYRV |
| 269 | ABL-CD3-43 | CSYAGSTTFAI |
| 270 | ABL-CD3-44/ABL-CD3-44A | GTWHSNSKTWV |
| 271 | ABL-CD3-45/ABL-CD3-45A | QQYKSYSWT |
| 272 | ABL-CD3-46/ABL-CD3-46A | YDRSNRV |
| 273 | ABL-CD3-47/ABL-CD3-47A | QSYYSNNRV |
| 274 | ABL-CD3-48/ABL-CD3-48A | QSFDSSNRV |
| 275 | ABL-CD3-49 | QCYDRSNRV |
| 276 | ABL-CD3-50/ABL-CD3-50A | QSFDTSNVV |
| 277 | ABL-CD3-51/ABL-CD3-51A | QSYDSSNRV |
| 278 | ABL-CD3-52/ABL-CD3-52A | YSTDSSGNHR V |
| 279 | ABL-CD3-53/ABL-CD3-53A | QSYDSSNRV |
| 280 | ABL-CD3-54/ABL-CD3-54A | QSFDSSNRV |
| 281 | ABL-CD3-55/ABL-CD3-55A | QSYDSSNRV |
| 282 | ABL-CD3-56/ABL-CD3-56A | QSYDSSNRV |
| 283 | ABL-CD3-57/ABL-CD3-57A | GTWHSNSKTQV |
| 284 | ABL-CD3-58/ABL-CD3-58A | QSFDSSNRV |
| 285 | ABL-CD3-59/ABL-CD3-59A | QSYDNSIRV |
| 286 | ABL-CD3-60/ABL-CD3-60A | QSYDSSNRV |
| 287 | ABL-CD3-61/ABL-CD3-61A | LLYYGGAYV |
| 288 | ABL-CD3-62/ABL-CD3-62A | QSYDSSNRV |
| 289 | ABL-CD3-63/ABL-CD3-63A | QSYDRSNRV |
| 290 | ABL-CD3-64/ABL-CD3-64A | QSYDSSNRV |
| 291 | ABL-CD3-65/ABL-CD3-65A | AAWDDSLSGVV |
| 292 | ABL-CD3-66/ABL-CD3-66A | QSYDRSNRV |
| 293 | ABL-CD3-67/ABL-CD3-67A | QSYDSSNRV |
| 294 | ABL-CD3-68/ABL-CD3-68A | QSADSSGTYVV |
| 295 | ABL-CD3-69/ABL-CD3-69A | QSYDSSNRV |
| 296 | ABL-CD3-70/ABL-CD3-70A | QSYDSSNRV |
| 297 | ABL-CD3-71/ABL-CD3-71A | QSYDRSNRV |
| 298 | ABL-CD3-72/ABL-CD3-72A | QSYDSSNRV |
| 299 | ABL-CD3-73/ABL-CD3-73A | QSYDNSNRV |
| 300 | ABL-CD3-74/ABL-CD3-74A | YSTDSSGNHWV |
| 301 | ABL-CD3-75/ABL-CD3-75A | YSTDSSGNHNWV |
| 302 | ABL-CD3-76/ABL-CD3-76A | YSTDSSGNHWV |
| 303 | ABL-CD3-77/ABL-CD3-77A | QSYDSSLSGSV |
| 304 | ABL-CD3-78/ABL-CD3-78A | QSFDGSNRV |
| 305 | ABL-CD3-79/ABL-CD3-79A | VLYMGSGIWV |
| 306 | ABL-CD3-80/ABL-CD3-80A | YSTDSSGNHSWV |
| 307 | ABL-CD3-81/ABL-CD3-81A | QSFDSSNRV |
| 308 | ABL-CD3-82/ABL-CD3-82A | YSTDSSGNHSWV |
| 309 | ABL-CD3-83/ABL-CD3-83A | QSSDRSNVV |
| 310 | ABL-CD3-84/ABL-CD3-84A | QSYDSSNRV |
| 311 | ABL-CD3-85 | QSCDSSNVV |
| 312 | ABL-CD3-86/ABL-CD3-86A | QSCDSSNVV |
| 313 | ABL-CD3-87/ABL-CD3-87A | QSYDSSNRV |
| 314 | ABL-CD3-88/ABL-CD3-88A | QQYNSYSWT |
| 315 | ABL-CD3-89/ABL-CD3-89A | QQYGSSRWS |
| 316 | ABL-CD3-90/ABL-CD3-90A | QSYDSSNQV |
| 317 | ABL-CD3-91/ABL-CD3-91A | QSYDSSNQV |
| 318 | ABL-CD3-92/ABL-CD3-92A | QAWDSTTAV |
| 319 | ABL-CD3-93/ABL-CD3-93A | QSYDSSNQV |
| 320 | ABL-CD3-94/ABL-CD3-94A | QSYDSSIRV |
| 321 | ABL-CD3-95/ABL-CD3-95A | QSYDSSNQV |
| 322 | ABL-CD3-96/ABL-CD3-96A | QSYDSSNVV |
| 323 | ABL-CD3-97/ABL-CD3-97A | QAWDSTTAV |
| 324 | ABL-CD3-98/ABL-CD3-98A | QSYDNNNRV |
| 325 | ABL-CD3-99/ABL-CD3-99A | QSYDSSIRV |
| 326 | ABL-CD3-100/ABL-CD3-100A | QAWDSITAV |
| 327 | ABL-CD3-101/ABL-CD3-101A | QSYDSSNQV |
| 328 | ABL-CD3-102/ABL-CD3-102A | QSYDRSIRV |
| 329 | ABL-CD3-103/ABL-CD3-103A | QSYDSSNVV |
| 330 | ABL-CD3-104/ABL-CD3-104A | QSYDSSNVV |
| 331 | ABL-CD3-105/ABL-CD3-105A | QSYDSSNQV |
| 332 | ABL-CD3-106/ABL-CD3-106A | QSYDGSIRV |
| 333 | ABL-CD3-107/ABL-CD3-107A | AAWDDSLNGRVV |
| 334 | ABL-CD3-108/ABL-CD3-108A | QSYDSSNVV |
| 335 | ABL-CD3-109/ABL-CD3-109A | QSYDSSNQV |
| 336 | ABL-CD3-110/ABL-CD3-110A | QSYDGSNRV |
| 337 | ABL-CD3-111/ABL-CD3-111A | QQYGSSPFT |
| 338 | ABL-CD3-112/ABL-CD3-112A | VLYMGSGIWV |
| 339 | ABL-CD3-113/ABL-CD3-113A | VLYMGSGIWV |
| 340 | ABL-CD3-41A | YSTDSSGNHSWV |
| 341 | ABL-CD3-43A | SSYAGSTTFAI |
| 342 | ABL-CD3-49A | QSYDRSNRV |
| 343 | ABL-CD3-85A | QSYDSSNVV |
| 344 | ABL-CD3-85B | QSSDSSNVV |

TABLE 1C
HCDR Sequences

| | | |
|---|---|---|
| SEQ | | |
| ID | | |
| NO: | Name | Sequence |

Heavy Chain CDR1 Sequences

| | | |
|---|---|---|
| 345 | ABL-CD3-1/ABL-CD3-1A | SYSMN |
| 346 | ABL-CD3-2/ABL-CD3-2A | SYSMN |
| 347 | ABL-CD3-3/ABL-CD3-3A | SYSMN |
| 348 | ABL-CD3-4/ABL-CD3-4A | SYNMN |
| 349 | ABL-CD3-5/ABL-CD3-5A | RYSMN |
| 350 | ABL-CD3-6/ABL-CD3-6A | SYSMN |
| 351 | ABL-CD3-7/ABL-CD3-7A | RYSMN |
| 352 | ABL-CD3-8/ABL-CD3-8A | SYSMN |
| 353 | ABL-CD3-9/ABL-CD3-9A | SYSMN |
| 354 | ABL-CD3-10/ABL-CD3-10A | CNSVA |
| 355 | ABL-CD3-11/ABL-CD3-11A | TFGMGVG |
| 356 | ABL-CD3-12/ABL-CD3-12A | SYTMN |
| 357 | ABL-CD3-13/ABL-CD3-13A | NYWMS |
| 358 | ABL-CD3-14/ABL-CD3-14A | SYSMT |
| 359 | ABL-CD3-15/ABL-CD3-15A | SYSMN |
| 360 | ABL-CD3-16/ABL-CD3-16A | RYWMT |
| 361 | ABL-CD3-17/ABL-CD3-17A | RYSIN |
| 362 | ABL-CD3-18/ABL-CD3-18A | SYAMH |
| 363 | ABL-CD3-19/ABL-CD3-19A | SYSMN |
| 364 | ABL-CD3-20/ABL-CD3-20A | TYSMN |
| 365 | ABL-CD3-21/ABL-CD3-21A | SYSMN |
| 366 | ABL-CD3-22/ABL-CD3-22A | SYSMN |
| 367 | ABL-CD3-23/ABL-CD3-23A | SYSMN |
| 368 | ABL-CD3-24/ABL-CD3-24A | SYTMN |
| 369 | ABL-CD3-25/ABL-CD3-25A | SYSMN |
| 370 | ABL-CD3-26/ABL-CD3-26A | SYSMN |
| 371 | ABL-CD3-27/ABL-CD3-27A | SYYMH |
| 372 | ABL-CD3-28/ABL-CD3-28A | SNSVA |
| 373 | ABL-CD3-29/ABL-CD3-29A | SYWIA |
| 374 | ABL-CD3-30/ABL-CD3-30A | SYYWS |
| 375 | ABL-CD3-31/ABL-CD3-31A | NYWIG |
| 376 | ABL-CD3-32/ABL-CD3-32A | SYNMN |
| 377 | ABL-CD3-33/ABL-CD3-33A | SYSMN |
| 378 | ABL-CD3-34/ABL-CD3-34A | RYSMN |
| 379 | ABL-CD3-35/ABL-CD3-35A | SYSMN |
| 380 | ABL-CD3-36/ABL-CD3-36A | NYWMS |
| 381 | ABL-CD3-37/ABL-CD3-37A | SYSMN |
| 382 | ABL-CD3-38/ABL-CD3-38A | GYYWS |
| 383 | ABL-CD3-39/ABL-CD3-39A | YYAMS |
| 384 | ABL-CD3-40/ABL-CD3-40A | YYAMS |
| 385 | ABL-CD3-41/ABL-CD3-41A | SYSMN |
| 386 | ABL-CD3-42/ABL-CD3-42A | SYSIN |
| 387 | ABL-CD3-43/ABL-CD3-43A | TYWIA |
| 388 | ABL-CD3-44/ABL-CD3-44A | TYWMS |
| 389 | ABL-CD3-45/ABL-CD3-45A | SYWMS |
| 390 | ABL-CD3-46/ABL-CD3-46A | TYTMN |
| 391 | ABL-CD3-47/ABL-CD3-47A | RYSMN |
| 392 | ABL-CD3-48/ABL-CD3-48A | SYTMN |
| 393 | ABL-CD3-49/ABL-CD3-49A | TYTMN |
| 394 | ABL-CD3-50/ABL-CD3-50A | SNSVA |
| 395 | ABL-CD3-51/ABL-CD3-51A | SYSMN |
| 396 | ABL-CD3-52/ABL-CD3-52A | YYSMN |
| 397 | ABL-CD3-53/ABL-CD3-53A | SYTMN |
| 398 | ABL-CD3-54/ABL-CD3-54A | SYSMN |
| 399 | ABL-CD3-55/ABL-CD3-55A | IYSMN |
| 400 | ABL-CD3-56/ABL-CD3-56A | RYSMN |
| 401 | ABL-CD3-57/ABL-CD3-57A | SYTMN |
| 402 | ABL-CD3-58/ABL-CD3-58A | NYNMN |
| 403 | ABL-CD3-59/ABL-CD3-59A | SYSMN |
| 404 | ABL-CD3-60/ABL-CD3-60A | IYSMN |
| 405 | ABL-CD3-61/ABL-CD3-61A | SYSMN |
| 406 | ABL-CD3-62/ABL-CD3-62A | SYSMN |
| 407 | ABL-CD3-63/ABL-CD3-63A | SYYWS |
| 408 | ABL-CD3-64/ABL-CD3-64A | RYSMN |
| 409 | ABL-CD3-65/ABL-CD3-65A | GYYWS |
| 410 | ABL-CD3-66/ABL-CD3-66A | SYYWS |
| 411 | ABL-CD3-67/ABL-CD3-67A | SYSMN |
| 412 | ABL-CD3-68/ABL-CD3-68A | SSIMN |
| 413 | ABL-CD3-69/ABL-CD3-69A | TYTMN |
| 414 | ABL-CD3-70/ABL-CD3-70A | TYIMN |
| 415 | ABL-CD3-71/ABL-CD3-71A | SYYWS |
| 416 | ABL-CD3-72/ABL-CD3-72A | TYIMN |
| 417 | ABL-CD3-73/ABL-CD3-73A | SYSMN |
| 418 | ABL-CD3-74/ABL-CD3-74A | SYSMK |
| 419 | ABL-CD3-75/ABL-CD3-75A | TYSLN |
| 420 | ABL-CD3-76/ABL-CD3-76A | NYYMN |
| 421 | ABL-CD3-77/ABL-CD3-77A | SYSMN |
| 422 | ABL-CD3-78/ABL-CD3-78A | NYSMN |
| 423 | ABL-CD3-79/ABL-CD3-79A | NYSMN |
| 424 | ABL-CD3-80/ABL-CD3-80A | GYSMN |
| 425 | ABL-CD3-81/ABL-CD3-81A | SGGYY |
| 426 | ABL-CD3-82/ABL-CD3-82A | SYSMN |
| 427 | ABL-CD3-83/ABL-CD3-83A | SYSMN |
| 428 | ABL-CD3-84/ABL-CD3-84A | NFIMN |
| 429 | ABL-CD3-85/ABL-CD3-85A | SYSMN |
| 430 | ABL-CD3-86/ABL-CD3-86A | RYSMN |
| 431 | ABL-CD3-87/ABL-CD3-87A | RYSMN |
| 432 | ABL-CD3-88/ABL-CD3-88A | RYWMS |
| 433 | ABL-CD3-89/ABL-CD3-89A | SYWMS |
| 434 | ABL-CD3-90/ABL-CD3-90A | SYSMN |
| 435 | ABL-CD3-91/ABL-CD3-91A | YYSMN |
| 436 | ABL-CD3-92/ABL-CD3-92A | SYSMN |
| 437 | ABL-CD3-93/ABL-CD3-93A | RYSMN |
| 438 | ABL-CD3-94/ABL-CD3-94A | SYSMN |
| 439 | ABL-CD3-95/ABL-CD3-95A | RYSMN |
| 440 | ABL-CD3-96/ABL-CD3-96A | RYSMN |
| 441 | ABL-CD3-97/ABL-CD3-97A | SYSMN |
| 442 | ABL-CD3-98/ABL-CD3-98A | SYSMN |
| 443 | ABL-CD3-99/ABL-CD3-99A | SYSMN |
| 444 | ABL-CD3-100/ABL-CD3-100A | SYSMN |
| 445 | ABL-CD3-101/ABL-CD3-101A | RYSMN |
| 446 | ABL-CD3-102/ABL-CD3-102A | SYSMN |
| 447 | ABL-CD3-103/ABL-CD3-103A | RYSMN |
| 448 | ABL-CD3-104/ABL-CD3-104A | RYSMN |
| 449 | ABL-CD3-105/ABL-CD3-105A | RYSMN |
| 450 | ABL-CD3-106/ABL-CD3-106A | SYSMN |
| 451 | ABL-CD3-107/ABL-CD3-107A | TYWIA |
| 452 | ABL-CD3-108/ABL-CD3-108A | RYSMN |
| 453 | ABL-CD3-109/ABL-CD3-109A | SYSMN |
| 454 | ABL-CD3-110/ABL-CD3-110A | SYSMN |
| 455 | ABL-CD3-111/ABL-CD3-111A | SYSMN |
| 456 | ABL-CD3-112/ABL-CD3-112A | SYSMN |
| 457 | ABL-CD3-113/ABL-CD3-113A | SYSMN |

Heavy Chain CDR2 Sequences

| | | |
|---|---|---|
| 458 | ABL-CD3-1/ABL-CD3-1A | SISRSGSYIYYADSVKG |
| 459 | ABL-CD3-2/ABL-CD3-2A | SISRSTSYIYYADSVKG |
| 460 | ABL-CD3-3/ABL-CD3-3A | YISRSSNYIHYADSVKG |
| 461 | ABL-CD3-4/ABL-CD3-4A | SISSSSSYIYYADSVKG |
| 462 | ABL-CD3-5/ABL-CD3-5A | YISSSSSSIYYADSVKG |
| 463 | ABL-CD3-6/ABL-CD3-6A | SISSRGSYRNYADSVKG |
| 464 | ABL-CD3-7/ABL-CD3-7A | SISSSGSYKYYADSVKG |
| 465 | ABL-CD3-8/ABL-CD3-8A | SISSSSSYIYYADSVKG |
| 466 | ABL-CD3-9/ABL-CD3-9A | SVNSRGSYIYYADSVKG |
| 467 | ABL-CD3-10/ABL-CD3-10A | LGRTYYRSKWHNEYAVSVK |
| 468 | ABL-CD3-11/ABL-CD3-11A | HIWWDDDKYYNPALKS |
| 469 | ABL-CD3-12/ABL-CD3-12A | SISRSSSYKYYADSVKG |
| 470 | ABL-CD3-13/ABL-CD3-13A | NIKEDGSEKYYVDSVKG |
| 471 | ABL-CD3-14/ABL-CD3-14A | SISRRSSYIYYADSVKG |
| 472 | ABL-CD3-15/ABL-CD3-15A | SISRSSNYIYYADSVKG |
| 473 | ABL-CD3-16/ABL-CD3-16A | NIKEDGSEKYCVDSVKG |
| 474 | ABL-CD3-17/ABL-CD3-17A | YISSSSSTIYYADSVKG |
| 475 | ABL-CD3-18/ABL-CD3-18A | AISSNGGRTYYADSVKG |
| 476 | ABL-CD3-19/ABL-CD3-19A | SISRSSSYIYYADSVKG |
| 477 | ABL-CD3-20/ABL-CD3-20A | SISSSGSYVYYADSVKG |
| 478 | ABL-CD3-21/ABL-CD3-21A | SISRSSSYIYYADSVKG |
| 479 | ABL-CD3-22/ABL-CD3-22A | YISSSSSTIYYADSVKG |
| 480 | ABL-CD3-23/ABL-CD3-23A | SISSSSTYIYYADSVKG |
| 481 | ABL-CD3-24/ABL-CD3-24A | SISRSGSYKYNADSVKG |
| 482 | ABL-CD3-25/ABL-CD3-25A | SISSSSTYIYYADSVKG |
| 483 | ABL-CD3-26/ABL-CD3-26A | SISSSSTYIYYADSVKG |
| 484 | ABL-CD3-27/ABL-CD3-27A | IINPSNGNTISAQKFQG |
| 485 | ABL-CD3-28/ABL-CD3-28A | LGRTYYRSKWYNECAGSVK |
| 486 | ABL-CD3-29/ABL-CD3-29A | IIYPGDSETRYRPSFQGQV |
| 487 | ABL-CD3-30/ABL-CD3-30A | HIYYSGRTKYNPSLKS |
| 488 | ABL-CD3-31/ABL-CD3-31A | IIYPADSDTTYSPSFQGQV |
| 489 | ABL-CD3-32/ABL-CD3-32A | SISSSSSYIYYTDSVKG |
| 490 | ABL-CD3-33/ABL-CD3-33A | SISRSSSYIYYADSVKG |
| 491 | ABL-CD3-34/ABL-CD3-34A | SISRSSSYIYYADSVKG |
| 492 | ABL-CD3-35/ABL-CD3-35A | SVNSRGSYIYYADSVKG |
| 493 | ABL-CD3-36 | NIKEDGSEKYCVDSVKG |
| 494 | ABL-CD3-37/ABL-CD3-37A | HIRSSTYSIYYADSVMG |
| 495 | ABL-CD3-38/ABL-CD3-38A | EINHSGNTNYNPSLKS |
| 496 | ABL-CD3-39/ABL-CD3-39A | AISGSGGSTYYADSVKG |
| 497 | ABL-CD3-40/ABL-CD3-40A | AISGSGGSTYYADSVKG |
| 498 | ABL-CD3-41/ABL-CD3-41A | SVNSRGSYIYYADSVKG |
| 499 | ABL-CD3-42/ABL-CD3-42A | YISSSSSTIYYADSVKG |
| 500 | ABL-CD3-43/ABL-CD3-43A | IIYPGDSDTTYSPSFQGQV |
| 501 | ABL-CD3-44/ABL-CD3-44A | NIKQDGSEKYYVDSVKG |
| 502 | ABL-CD3-45/ABL-CD3-45A | NIKEDGSEKYYVDSVKG |
| 503 | ABL-CD3-46/ABL-CD3-46A | SISKSSTYIYYADSVKG |
| 504 | ABL-CD3-47/ABL-CD3-47A | YISRSSTTIYYADSVKG |
| 505 | ABL-CD3-48/ABL-CD3-48A | SISGSGSYIYYADSVKG |
| 506 | ABL-CD3-49/ABL-CD3-49A | SISKSSSYIYYTDSVKG |
| 507 | ABL-CD3-50/ABL-CD3-50A | LGRTYSRSKWYNDYAVSVK |
| 508 | ABL-CD3-51/ABL-CD3-51A | SISRSSSYIYYADSVKG |
| 509 | ABL-CD3-52/ABL-CD3-52A | SISRSTTYIYYADSVKG |
| 510 | ABL-CD3-53/ABL-CD3-53A | SITTSSSYIYYADSVKG |
| 511 | ABL-CD3-54/ABL-CD3-54A | SISRSSRYIFYADSVRG |
| 512 | ABL-CD3-55/ABL-CD3-55A | SISSGSSYIYYADSVKG |
| 513 | ABL-CD3-56/ABL-CD3-56A | SISSSSRYTYYADSLKG |
| 514 | ABL-CD3-57/ABL-CD3-57A | SISTSSRYIYYTDSVKG |
| 515 | ABL-CD3-58/ABL-CD3-58A | SSSRSSYIYYADSVKG |
| 516 | ABL-CD3-59/ABL-CD3-59A | SLSRSSNYIYYAVSVKG |
| 517 | ABL-CD3-60/ABL-CD3-60A | SISSGSSYIYYADSVKG |
| 518 | ABL-CD3-61/ABL-CD3-61A | SISRSGTYIYYADSVKG |
| 519 | ABL-CD3-62/ABL-CD3-62A | SISRSSGYIFYADSVKG |
| 520 | ABL-CD3-63/ABL-CD3-63A | RIYTSGSTKYNPSLKS |
| 521 | ABL-CD3-64/ABL-CD3-64A | SISSSGSYTYYADSVKG |
| 522 | ABL-CD3-65 | EINHSGSTNYNPSLKS |
| 523 | ABL-CD3-66/ABL-CD3-66A | RIYASGSTYYNPSLKS |
| 524 | ABL-CD3-67/ABL-CD3-67A | SISRSSSYIYYADSVKG |
| 525 | ABL-CD3-68/ABL-CD3-68A | SISGSSSYIYYADSVKG |
| 526 | ABL-CD3-69/ABL-CD3-69A | TITSSSRYIYYADSVKG |
| 527 | ABL-CD3-70/ABL-CD3-70A | SITSSSSYIYYADSVKG |
| 528 | ABL-CD3-71/ABL-CD3-71A | RIYTSGSTNYNPSLKS |
| 529 | ABL-CD3-72/ABL-CD3-72A | SISSSRSYIYYADSVKG |
| 530 | ABL-CD3-73/ABL-CD3-73A | TFSRSSTYIYYADSVKG |
| 531 | ABL-CD3-74/ABL-CD3-74A | SISSSSSYIYYADSVKG |
| 532 | ABL-CD3-75/ABL-CD3-75A | SISVSSRYIYFADSVKG |
| 533 | ABL-CD3-76/ABL-CD3-76A | SISSSSNYIYYADSVKG |
| 534 | ABL-CD3-77/ABL-CD3-77A | SISSSSSYIYYADSVKG |
| 535 | ABL-CD3-78/ABL-CD3-78A | CISRSSSYIYYADSVKG |
| 536 | ABL-CD3-79 | CISRSSSYIYYADSVKG |
| 537 | ABL-CD3-80/ABL-CD3-80A | SISSRSRYIYYADSVQG |
| 538 | ABL-CD3-81/ABL-CD3-81A | IGYIYYSGSTYYNPSLK |
| 539 | ABL-CD3-82/ABL-CD3-82A | SISSSSSYIYYADSVKG |
| 540 | ABL-CD3-83/ABL-CD3-83A | TISRSGGYIYDADSVKG |
| 541 | ABL-CD3-84/ABL-CD3-84A | SISGSSNYIYYADSVKG |
| 542 | ABL-CD3-85/ABL-CD3-85A | SISRRSGYIYYADSVKG |
| 543 | ABL-CD3-86/ABL-CD3-86A | TISRSSRYIYYGDSVKG |
| 544 | ABL-CD3-87/ABL-CD3-87A | SISSSSSYKYYADSVKG |
| 545 | ABL-CD3-88/ABL-CD3-88A | NIKEDGSEKYYVDSVKG |
| 546 | ABL-CD3-89/ABL-CD3-89A | SISSRRGYKYHADSVKG |
| 547 | ABL-CD3-90/ABL-CD3-90A | SISSSSSYIYYADSVKG |
| 548 | ABL-CD3-91/ABL-CD3-91A | SISSSSTYIYYADSVKG |
| 549 | ABL-CD3-92/ABL-CD3-92A | YISSSSRYIYYADSVKG |
| 550 | ABL-CD3-93/ABL-CD3-93A | SISSSSSYIYYADSVKG |
| 551 | ABL-CD3-94/ABL-CD3-94A | SISRSSSYIFYADSVKG |
| 552 | ABL-CD3-95/ABL-CD3-95A | SISRSSSYIYYADSVKG |
| 553 | ABL-CD3-96/ABL-CD3-96A | SISSSSSYIYYADSVKG |
| 554 | ABL-CD3-97/ABL-CD3-97A | YISRSSTYIYYADSMKG |
| 555 | ABL-CD3-98/ABL-CD3-98A | SISSSSSYIYYADSVKG |
| 556 | ABL-CD3-99/ABL-CD3-99A | SISSSSSYIYYADSVKG |
| 557 | ABL-CD3-100/ABL-CD3-100A | YISSSSNYIYYADSVKG |
| 558 | ABL-CD3-101/ABL-CD3-101A | SISSSSSYIYYADSVKG |
| 559 | ABL-CD3-102/ABL-CD3-102A | SISSSSSYIYYADSVKG |
| 560 | ABL-CD3-103/ABL-CD3-103A | SISSSSSYISYADSVKG |
| 561 | ABL-CD3-104/ABL-CD3-104A | SISSSSSYIYYADSVKG |
| 562 | ABL-CD3-105/ABL-CD3-105A | SISSSSSYIYYADSVKG |
| 563 | ABL-CD3-106/ABL-CD3-106A | SISRSSSYIFYADSVKG |
| 564 | ABL-CD3-107/ABL-CD3-107A | FIYPGDSDTIYSPSFQGQV |
| 565 | ABL-CD3-108/ABL-CD3-108A | SISSSSRYIYYADSVKG |
| 566 | ABL-CD3-109/ABL-CD3-109A | SISSSSSYIYYADSVKG |
| 567 | ABL-CD3-110/ABL-CD3-110A | SISSSSSYIYYADSVKG |
| 568 | ABL-CD3-111/ABL-CD3-111A | YISRSSTYIYYADSMKG |
| 569 | ABL-CD3-112/ABL-CD3-112A | SISSSSSYIYYADSVKG |
| 570 | ABL-CD3-113/ABL-CD3-113A | SISSSSSYIYYADSVKG |
| 571 | ABL-CD3-36A | NIKEDGSEKYYVDSVKG |
| 572 | ABL-CD3-65A | EIQHSGSTNYNPSLKS |
| 573 | ABL-CD3-79A | SISRSSSYIYYADSVKG |

Heavy Chain CDR3 Sequences

| | | |
|---|---|---|
| 574 | ABL-CD3-1/ABL-CD3-1A | EKWELLYFDY |
| 575 | ABL-CD3-2/ABL-CD3-2A | GSRDAFDI |
| 576 | ABL-CD3-3/ABL-CD3-3A | AERGSYAVDI |
| 577 | ABL-CD3-4/ABL-CD3-4A | AERGSYAFDI |
| 578 | ABL-CD3-5/ABL-CD3-5A | DRRATLDY |
| 579 | ABL-CD3-6/ABL-CD3-6A | DRRELLLDC |
| 580 | ABL-CD3-7/ABL-CD3-7A | EPPTLGMDY |
| 581 | ABL-CD3-8/ABL-CD3-8A | DRRELLLDY |
| 582 | ABL-CD3-9/ABL-CD3-9A | DRRELLLDY |
| 583 | ABL-CD3-10/ABL-CD3-10A | ERKWELFLDY |
| 584 | ABL-CD3-11/ABL-CD3-11A | LGYYPYWFFDV |
| 585 | ABL-CD3-12/ABL-CD3-12A | EPPTLGMDY |
| 586 | ABL-CD3-13/ABL-CD3-13A | ERRTGYFDY |
| 587 | ABL-CD3-14/ABL-CD3-14A | ERWELLYFDY |
| 588 | ABL-CD3-15/ABL-CD3-15A | ERWIQLYSDY |
| 589 | ABL-CD3-16/ABL-CD3-16A | ERRTGYFDY |
| 590 | ABL-CD3-17/ABL-CD3-17A | ERWELLYFDY |
| 591 | ABL-CD3-18/ABL-CD3-18A | GGSLDY |
| 592 | ABL-CD3-19/ABL-CD3-19A | ERWELLYFDY |
| 593 | ABL-CD3-20/ABL-CD3-20A | ERRELLFDY |
| 594 | ABL-CD3-21/ABL-CD3-21A | DGKGAIFLDY |
| 595 | ABL-CD3-22/ABL-CD3-22A | DGPVDYGMDV |
| 596 | ABL-CD3-23/ABL-CD3-23A | EPRTLGMDY |
| 597 | ABL-CD3-24/ABL-CD3-24A | EPPTLGMDY |
| 598 | ABL-CD3-25/ABL-CD3-25A | EPRTLGMDY |
| 599 | ABL-CD3-26/ABL-CD3-26A | EPRTLGMDY |
| 600 | ABL-CD3-27 | CWGYYVMDV |
| 601 | ABL-CD3-28/ABL-CD3-28A | ERKWELFLDY |
| 602 | ABL-CD3-29/ABL-CD3-29A | QGAYDGFDI |
| 603 | ABL-CD3-30/ABL-CD3-30A | ENRNGAFDI |
| 604 | ABL-CD3-31/ABL-CD3-31A | HGGSYILDAFDI |
| 605 | ABL-CD3-32/ABL-CD3-32A | AERGSYAFDF |
| 606 | ABL-CD3-33/ABL-CD3-33A | EPPTLGMDY |
| 607 | ABL-CD3-34/ABL-CD3-34A | EPRTLGMDY |
| 608 | ABL-CD3-35/ABL-CD3-35A | DRRELLLDY |
| 609 | ABL-CD3-36/ABL-CD3-36A | ERRTGYFDY |
| 610 | ABL-CD3-37/ABL-CD3-37A | AKTGGDAFDI |
| 611 | ABL-CD3-38/ABL-CD3-38A | EVEIWGYFDY |
| 612 | ABL-CD3-39/ABL-CD3-39A | EGIVGARAEYFQH |
| 613 | ABL-CD3-40/ABL-CD3-40A | EGIVGARAEYFQH |
| 614 | ABL-CD3-41/ABL-CD3-41A | DRRELLLDY |
| 615 | ABL-CD3-42/ABL-CD3-42A | DGPVDYGMDV |
| 616 | ABL-CD3-43/ABL-CD3-43A | QGELDAFDI |
| 617 | ABL-CD3-44/ABL-CD3-44A | GSFDY |
| 618 | ABL-CD3-45/ABL-CD3-45A | ERRTGYLDY |
| 619 | ABL-CD3-46/ABL-CD3-46A | ERRTLAFDI |
| 620 | ABL-CD3-47/ABL-CD3-47A | ERWELLYFDY |
| 621 | ABL-CD3-48/ABL-CD3-48A | EPPTLGMDY |
| 622 | ABL-CD3-49/ABL-CD3-49A | ERRTLAFDI |
| 623 | ABL-CD3-50/ABL-CD3-50A | ERKWELFLDY |
| 624 | ABL-CD3-51/ABL-CD3-51A | ERWELLYFDY |
| 625 | ABL-CD3-52/ABL-CD3-52A | DGKGAIFLDY |
| 626 | ABL-CD3-53/ABL-CD3-53A | VTFDAFDI |
| 627 | ABL-CD3-54/ABL-CD3-54A | EKRELLFDY |
| 628 | ABL-CD3-55/ABL-CD3-55A | EKRELLFDY |
| 629 | ABL-CD3-56/ABL-CD3-56A | NLYNALDY |
| 630 | ABL-CD3-57/ABL-CD3-57A | NWGYDVFDI |
| 631 | ABL-CD3-58/ABL-CD3-58A | EKWELLFFDY |
| 632 | ABL-CD3-59/ABL-CD3-59A | ERWELLYFDY |
| 633 | ABL-CD3-60/ABL-CD3-60A | EKRELLFDY |
| 634 | ABL-CD3-61/ABL-CD3-61A | ERWELLYFDY |
| 635 | ABL-CD3-62/ABL-CD3-62A | ERVGAGFDY |
| 636 | ABL-CD3-63/ABL-CD3-63A | ERIRDLYFDY |
| 637 | ABL-CD3-64/ABL-CD3-64A | ERGKGAGMDV |
| 638 | ABL-CD3-65/ABL-CD3-65A | EYYYYGMDV |
| 639 | ABL-CD3-66/ABL-CD3-66A | EKRSRLYFDY |
| 640 | ABL-CD3-67/ABL-CD3-67A | ERWELLYFDY |
| 641 | ABL-CD3-68/ABL-CD3-68A | ERWNLLYFDY |
| 642 | ABL-CD3-69/ABL-CD3-69A | VTFDAFDI |
| 643 | ABL-CD3-70/ABL-CD3-70A | EKWELLFFDY |
| 644 | ABL-CD3-71/ABL-CD3-71A | ERISRLYFDY |
| 645 | ABL-CD3-72/ABL-CD3-72A | EDRYRLFFDY |
| 646 | ABL-CD3-73/ABL-CD3-73A | EKWDLLYFDY |
| 647 | ABL-CD3-74/ABL-CD3-74A | DRGELTLWY |
| 648 | ABL-CD3-75/ABL-CD3-75A | DRRELLLDY |
| 649 | ABL-CD3-76/ABL-CD3-76A | DRRELPLDY |
| 650 | ABL-CD3-77/ABL-CD3-77A | DGELGIGTYFDY |
| 651 | ABL-CD3-78/ABL-CD3-78A | ERWELLYFDY |
| 652 | ABL-CD3-79/ABL-CD3-79A | ERWELLYFDY |
| 653 | ABL-CD3-80/ABL-CD3-80A | DWRELLLDY |
| 654 | ABL-CD3-81/ABL-CD3-81A | ERKTSLPFDI |
| 655 | ABL-CD3-82/ABL-CD3-82A | DRRELLLDY |
| 656 | ABL-CD3-83/ABL-CD3-83A | DFNWTFDN |
| 657 | ABL-CD3-84/ABL-CD3-84A | EKWVLLFFDY |
| 658 | ABL-CD3-85/ABL-CD3-85A | DLNWAFDY |
| 659 | ABL-CD3-86/ABL-CD3-86A | DHNWSFDY |
| 660 | ABL-CD3-87/ABL-CD3-87A | EGGNSHGMDV |
| 661 | ABL-CD3-88/ABL-CD3-88A | ERRTGYFDY |
| 662 | ABL-CD3-89/ABL-CD3-89A | VPYSGSYFDY |
| 663 | ABL-CD3-90/ABL-CD3-90A | YRYNWNDY |
| 664 | ABL-CD3-91/ABL-CD3-91A | YRYNWNDY |
| 665 | ABL-CD3-92/ABL-CD3-92A | EGPNWGTHAFDI |
| 666 | ABL-CD3-93/ABL-CD3-93A | YRYNWNDY |
| 667 | ABL-CD3-94/ABL-CD3-94A | ERRNGFDY |
| 668 | ABL-CD3-95/ABL-CD3-95A | YRYNWNDY |
| 669 | ABL-CD3-96/ABL-CD3-96A | AVYNYFDQ |
| 670 | ABL-CD3-97/ABL-CD3-97A | EGPNWGTHAFDI |
| 671 | ABL-CD3-98/ABL-CD3-98A | ERRELAFDI |
| 672 | ABL-CD3-99/ABL-CD3-99A | ERRELSFDY |
| 673 | ABL-CD3-100/ABL-CD3-100A | EGPNWGTHAFDI |
| 674 | ABL-CD3-101/ABL-CD3-101A | YRYNWNDY |
| 675 | ABL-CD3-102/ABL-CD3-102A | ERRELSFDY |
| 676 | ABL-CD3-103/ABL-CD3-103A | VSYNSFDY |
| 677 | ABL-CD3-104/ABL-CD3-104A | AVYNYFDQ |
| 678 | ABL-CD3-105/ABL-CD3-105A | YRYNWNDY |
| 679 | ABL-CD3-106/ABL-CD3-106A | ERRNGFDY |
| 680 | ABL-CD3-107/ABL-CD3-107A | QGYLDAFDI |
| 681 | ABL-CD3-108/ABL-CD3-108A | ALYNYFDY |
| 682 | ABL-CD3-109/ABL-CD3-109A | YRYNWNDY |
| 683 | ABL-CD3-110/ABL-CD3-110A | ERRDLAFDI |
| 684 | ABL-CD3-111/ABL-CD3-111A | EGPNWGTHAFDI |
| 685 | ABL-CD3-112/ABL-CD3-112A | ERRELSFDY |
| 686 | ABL-CD3-113/ABL-CD3-113A | ERRELSFDY |
| 687 | ABL-CD3-27A | SWGYYVMDV |

TABLE 1D
VL Sequences

| | | |
|---|---|---|
| SEQ ID NO: | Name | Sequence |

Light Chain Variable Region Sequences

| | | |
|---|---|---|
| 688 | ABL-CD3-1 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 689 | ABL-CD3-2 | SYELTQPPSVSVSPGQTASITCSGDKLGNKYACWYQQKPGQSP |
| | | VLVIYQDSKRPSGIPERFSGSNSGNTATLTISGTQAMDEADYYC |
| | | QAWDISTVVFGGGTKLTVL |
| 690 | ABL-CD3-3 | SYELTQPPSVSVSPGQTASITCSGDKLRDKYACWYQQKPGQSP |
| | | VLVIYQDSKRPSGIPERFSGSNSGNTATLTISGTQAMDEADYYC |
| | | QAWDSSTVVFGGGTKLTVL |
| 691 | ABL-CD3-4 | SYELTQPPSVSVSPGQTASITCSGDKLGDKYAGWYQQKPGQSP |
| | | VLVIYQDSKRPSGIPERFSGSNSGNTATLTISGTQAMDEADYYC |
| | | QAWDSSTVVFGGGTKLTVL |
| 692 | ABL-CD3-5 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNHVFGGGTKLTVL |
| 693 | ABL-CD3-6 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGGGTKLTVL |
| 694 | ABL-CD3-7 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGGGTKLTVL |
| 695 | ABL-CD3-8 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGGGTKLTVL |
| 696 | ABL-CD3-9 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHCWVFGGGTKLTVL |
| 697 | ABL-CD3-10 | NFMLTQPHSVSESPGKTVTISCTRSSGSIASNYVQWYQQRPGSS |
| | | PTTMIYEDNQRPSGVPDRFSGSIDSSSNSASLTISGLKTEDEADY |
| | | YCQSLDSSSVVFGGGTKLTVL |
| 698 | ABL-CD3-11 | NFMLTQPHSVSESPGKTVTIACTRSSGSIASNYVQWYQQRPGSS |
| | | PTTVIYEDNQRPSGVPDRFSGSIDSSSNSASLTISGLKTEDEADY |
| | | YCQSYDSSNQVFGGGTKLTVL |
| 699 | ABL-CD3-12 | NFMLTQPHSVSESPGKTVTISCTRSNGSITSNYVQWYQQRPGSS |
| | | PTTVIYEDNQRPSGVPDRFSGSIDSSSNSASLTISGLKTEDEADY |
| | | YCQSFDNSNRVFGEKTKLTVL |
| 700 | ABL-CD3-13 | DIQMTQSPSTLSASVGDRVTITCRASQSISNWLAWYQQKPGKA |
| | | PNLLIYKASSLESGVPSRFSGSGSGTEFTLTISSLQPDDFANYYC |
| | | QQYKSYSWTFGQGTKVEIK |
| 701 | ABL-CD3-14 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 702 | ABL-CD3-15 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 703 | ABL-CD3-16 | DIQMTQSPSTLSASVGDRVTITCRASQSIGSWLAWYQQKPGKA |
| | | PKLLIYKASSLESGVPSRFSGSGSGTEFTLTISSLQPDDFATYYC |
| | | QQYNSYSWTFGQGTKVEIK |
| 704 | ABL-CD3-17 | NFMLTQPHSVSESPGKTVTISCTRSSGSIASKYVQWYQQRPGSS |
| | | PTTVIYEDNQRPSGVPDRFSGSIDSSSNSASLTISGLKTEDEADY |
| | | YCQSYDSNNRVFGGGTKLTVL |
| 705 | ABL-CD3-18 | DIVMTQTPLSSPVTPGQPASISCRSSQSLVHSDGNTYLNWLQQR |
| | | PGQPPRLLIYKISDRFSGVPDRFSGSGAGTDFTLKIGRVEAEDVG |
| | | VYYCMQSTQFPITFGQGTRLEIK |
| 706 | ABL-CD3-19 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGGGTKLTVL |
| 707 | ABL-CD3-20 | NFMLTQPHSVSESPGKTVTISCTRSSGSIASNYVQWYQQRPGSS |
| | | PTSVIYEDNQRPSGVPDRFSGSIDSSSNSASLTISGLKTEDEADY |
| | | YCQSYDRSNRVFGGGTKLTVL |
| 708 | ABL-CD3-21 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTHSSGNHRVFGGGTKLTVL |
| 709 | ABL-CD3-22 | SYELTQPPSVSVSPGQTARITCSGDALPKQYTYWCQQKPGQAP |
| | | VLVIYKDSERPSGIPERFSGSSSGTTVTLTISGVQAEDEADYYCQ |
| | | SADSSGTYRVFGGGTKLTVL |
| 710 | ABL-CD3-23 | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGGGTKLTVL |
| 711 | ABL-CD3-24 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFYSSNRVFGGGTELTVL |
| 712 | ABL-CD3-25 | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGGGTKLTVL |
| 713 | ABL-CD3-26 | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGGGTKLTVL |
| 714 | ABL-CD3-27 | QTVVTQEPSFSVSPGGTVTLTCGLNSGSVSTSYYPSWYQQTPG |
| | | QTPRTLIYSTNTRSSGVPDRFSGSILGNKAALTITGAQADDESDY |
| | | YCVLYMGSGIWVFGGGTKLTVL |
| 715 | ABL-CD3-28 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDSSNVVFGGGTKLTVL |
| 716 | ABL-CD3-29 | QSVLTQPPSASGTPGQRVTISCSGSSSNIGSNYVYWYQQLPGTA |
| | | PKLLIYSNNQRPSGVPDRFSGSKSGTSASLAISGLRSEDEADYYC |
| | | EAWDDSLSGPVFGGGTKLTVL |
| 717 | ABL-CD3-30 | NFMLTQPHSVSESPGKTVTISCTRSSGSIASNYVQWYQHRPGSS |
| | | PTTVIYEDDQRPSGIPDRFSGSIDSSSNSASLTISGLKTEDEADYY |
| | | CQSYNSSNQVFGGGTKLTVL |
| 718 | ABL-CD3-31 | DIVMTQSPLSLPVTPGEPASISCRSSQSLLHSNGYNYLDWYLQK |
| | | PGQSPQLLIYLGSNRASGVPDRFSGSGSGTDFTLKISRVEAEDV |
| | | GFYYCMQPLQTPYTFGQGTKLEIK |
| 719 | ABL-CD3-32 | SYELTQPPSVSVSPGQTASITCSGDKLGYKYASWYQQKPGQSP |
| | | VLVIYQDSKRPSGIPERFSGSNSGNTATLTISGTQAMDEADYYC |
| | | QAWDSSTVVFGGGTKLTVL |
| 720 | ABL-CD3-33 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYYNSNRVFGGGTKLTVL |
| 721 | ABL-CD3-34 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 722 | ABL-CD3-35 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHCWVFGGGTKLTVL |
| 723 | ABL-CD3-36 | DIQMTQSPSTLSASVGDRVTITCRASQSISSWLAWFQQKTGKAP |
| | | KLLIYKASSLESGVPSRFSGSGSGTEFTLTISSLQPDDFATYYCQ |
| | | QYISYSWTFGQGTKVEIK |
| 724 | ABL-CD3-37 | DIQMTQSPSTLSASVGDRVTITCRASQSISSWLAWYQQKPGKAP |
| | | KLLIHKASSLESGVPSRFSGSGSGTEFTLTISSLQPDDFATYYCQ |
| | | QYNSYWTFGQGTKVEIK |
| 725 | ABL-CD3-38 | QSVLTQPPSVSGAPGQRVTISCTGSSSNIGAGYDVHWYQQLPG |
| | | TAPKLLIYGNSNRPSGVPDRFSGSKSGTSASLAITGLQAEDETD |
| | | YNCQSYDSSLSGYVFGTGTKVTVL |
| 726 | ABL-CD3-39 | DVVMTQSPLSLPVTLGQPASISCRSSQSLVYSDGNTYLTWFQQ |
| | | RPGQSPRRLIYKVSNRDSGVPDRFSGSGSGTDFTLKISRVEAED |
| | | VEIYYCMQGTHWPLTFGGGTKVEIK |
| 727 | ABL-CD3-40 | QSVLTQPPSASGTPGQRVTISCSGSSSNIGSNTVNWYQQFPGTA |
| | | PKLLIYSNNQRPSGVPDRFSGSKSGTSASLAISGLQSEDEADYY |
| | | CAPWDDSLNGVVFGGGTKLTVL |
| 728 | ABL-CD3-41 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHCWVFGGGTKLTVL |
| 729 | ABL-CD3-42 | SYELTQPPSVSVSPGQTARITCSGDALPKQYTYWCQQKPGQAPI |
| | | LVIYKDSERPSGIPERFSGSSSGTTVTLTISGVQADDEADYYCQS |
| | | ADSSGTYRVFGGGTKLTVL |
| 730 | ABL-CD3-43 | QSALTQPASVSGSPGQSITISCTGTSSDVGSYNLVSWYQQHPGK |
| | | APKLMIYEDIRRPSGVSNRFSASKSGDTASLTISGLQAEDEADY |
| | | YCCSYAGSTTFAIFGGGTKLTVL |
| 731 | ABL-CD3-44 | QPVLTQPSSHSASSGASVRLTCMLSSGFSVGDFWIRWYQQKPG |
| | | NPPRYLLFYHSDSNKGQGSGVPSRFSGSNDASANAGILRISGLQ |
| | | PEDEADYYCGTWHSNSKTWVFGGGTKLTVL |
| 732 | ABL-CD3-45 | DIQMTQSPSTLSASVGDRVTITCRASQSISSWLAWYQQKPGKAP |
| | | NLLIYKASSLESGVPSRFSGSGSGTEFTLTISSLQPDDFATYYCQ |
| | | QYKSYSWTIGQGTKVEIK |
| 733 | ABL-CD3-46 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQCYDRSNRVFGGGTKLTVL |
| 734 | ABL-CD3-47 | NFMLTQPHSVSESPGKTVTISCTRSSGSIASKYVQWYQQRPGSS |
| | | PTTVIYEDNQRPSGVPDRFSGSIDSSSNSASLTISGLKTEDEADY |
| | | YCQSYYSNNRVFGGGTKLTVL |
| 735 | ABL-CD3-48 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDSSNRVFGGGTKLTVL |
| 736 | ABL-CD3-49 | NFMLTQPHSVSESPGKTVTISCTRSSGSIASNYVQWYQQRPGSS |
| | | PTTVIYEDNQRSSGVPDRFSGSIDSSSNSASLTISGLKTEDEADY |
| | | YCQCYDRSNRVFGGGTKLTVL |
| 737 | ABL-CD3-50 | NFMLTQPHSVSESPGKTVTISCTRSSGSIASNYVQWYQQRPGISP |
| | | TTVIYEDNQRPSGVPDRFSGSIDSSSNSASLTISGLKTEDEADYY |
| | | CQSFDTSNVVFGGGTKLTVL |
| 738 | ABL-CD3-51 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 739 | ABL-CD3-52 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHRVFGGGTKLTVL |
| 740 | ABL-CD3-53 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 741 | ABL-CD3-54 | NSMLTQPHSVSESPGKTVTISCTRSSGSIASYYVQWYQQRPGSS |
| | | PTTVIYEDNQRPSGVPDRFSGSIDSSSNSASLTISGLKTEDEADY |
| | | YCQSFDSSNRVFGGGTKLTVL |
| 742 | ABL-CD3-55 | NFMLTQPHSVSESPGKTVTISCTRSSGSIASNYVQWYQQRPGSS |
| | | PTTVIYEDNQRPSGVPDRFSGSIDRSSNSASLTISGLKTEDEADY |
| | | YCQSYDSSNRVFGGGTKLTVL |
| 743 | ABL-CD3-56 | FMLTQPHSVSESPGKTVTISCTRSSGSIASNYVQWYQQRPGSSP |
| | | TTVIYEDNQRPSGVPDRFSGSIDSSSNSASLTISGLQTEDEADYY |
| | | CQSYDSSNRVFGGGTKLTVL |
| 744 | ABL-CD3-57 | QPVLTQPSSHSASSGASVRLTCMLSSGFSVGDFWIRWYQQKPG |
| | | NPPRYLLYYHSDSNKGQGSGVPSRFSGSNDASANAGILRISGLQ |
| | | PEDEADYYCGTWHSNSKTQVFGGGTKLTVL |
| 745 | ABL-CD3-58 | NFMLTQPHSVSESPGKTVTISCTRSSGSIASNFVQWYQQRPGSS |
| | | PTTVIYEDNQRPSGVPDRFSGSIDSSSNSASLTISGLKTEDEADY |
| | | YCQSFDSSNRVFGGGTKLTVL |
| 746 | ABL-CD3-59 | NFMLTQPHSVSESPGKTVTISCTRSSGSIASNYVQWYQQRPGSS |
| | | PTTVIYEDNQRPSGVPDRFSGSIDSSSNSASLTISGLQTEDEADY |
| | | YCQSYDNSIRVFGGGTKLTVL |
| 747 | ABL-CD3-60 | NFMLTQPHSVSESPGKTVTISCTRSSGSIASNYVQWYQQRPGSS |
| | | PTTVIYEDNQRPSGVPDRFSGSIDRSSNSASLTISGLKTEDEADY |
| | | YCQSYDSSNRVFGGGTKLTVL |
| 748 | ABL-CD3-61 | QTVVTQEPSLTVSPGGTVTLTCASSTGAVTSGYYPNWFQQKPG |
| | | QAPRALIYSTSNKHSWTPARFSGSLLGGKAALTLSGVQPEDEA |
| | | EYYCLLYYGGAYVFGTGTKVTVL |
| 749 | ABL-CD3-62 | NFMLTQPHSVSESPGKTVAISCTRSSGSIASNYVQWYQQRPGSS |
| | | PTAVIYEDNQRPSGVPDRFSGSIDSSSNSAFLTISGLKTEDEADY |
| | | YCQSYDSSNRVFGGGTKLTVL |
| 750 | ABL-CD3-63 | NFMLTQPHSVSESPGKTVTISCTRTSGSIASNYVQWYQQRPGSS |
| | | PTTVIYEDNQRPSGVPDRFSGSIDSSSNSASLTISGLKTEDEAAY |
| | | YCQSYDRSNRVFGGGTKLTVL |
| 751 | ABL-CD3-64 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 752 | ABL-CD3-65 | QSVLTQPPSASGTPGQRVTISCSGSSSNIGSNYVYWYQQLPGTA |
| | | PKLLIYSNNQRPSGVPDRFSGSKSGTSASLAISGLRSEDEADYYC |
| | | AAWDDSLSGVVFGGGTKLTVL |
| 753 | ABL-CD3-66 | NFMLTQPHSVSESPGKTVTISCTRSSGSIASNYVQWYQQRPGSS |
| | | PTTVIYEDNQRPSGVPDRFSGSIDSSSNSASLTISGLKTEDEGDY |
| | | YCQSYDRSNRVFGGGTKLTVL |
| 754 | ABL-CD3-67 | NFMLTQPHSVSESPGKTVTISCTRSSGSIASNYVQWYRQRPGSS |
| | | PTTVIYEDNQRPSGVPDRFSGAIDSSSTSASLTISGLKTEDEADY |
| | | YCQSYDSSNRVFGGGTKLTVL |
| 755 | ABL-CD3-68 | SYELTQPPSVSVSPGQTARITCSGDALPKQYAYWYQQKPGQAP |
| | | VLVIYKDSERPSGIPERFSGSSSGTTVTLTISGVQAEDEADYYCQ |
| | | SADSSGTYVVFGGGTKLTVL |
| 756 | ABL-CD3-69 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 757 | ABL-CD3-70 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 758 | ABL-CD3-71 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDRSNRVFGGGTKLTVL |
| 759 | ABL-CD3-72 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 760 | ABL-CD3-73 | NFMLTQPHSVSESPGKTVTISCTRSSGSIASNYVQWYQQRPGSS |
| | | PTTVIYEDNRRPSGVPDRFSGSIDSSSNSASLTISGLKTEDEADY |
| | | YCQSYDNSNRVFGGGTTLTVL |
| 761 | ABL-CD3-74 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHWVFGGGTKLTVL |
| 762 | ABL-CD3-75 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHNWVFGGGTKLTVL |
| 763 | ABL-CD3-76 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHWVFGGGTKLTVL |
| 764 | ABL-CD3-77 | QSVLTQPPSVSGAPGQRVTISCTGSSSNIGAGYDVHWYQQLPG |
| | | TAPKLLIYGNSNRPSGVPDRFSGSKSGTSASLAITGLQAEDEAD |
| | | YYCQSYDSSLSGSVFGGGTKLTVL |
| 765 | ABL-CD3-78 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDGSNRVFGGGTKLTVL |
| 766 | ABL-CD3-79 | QTVVTQEPSFSVSPGGTVTLTCGLSSGSVSTSYYPSWYQQTPGQ |
| | | APRTLIYSTNTRSSGVPDRFSGSILGNKAALTITGAQADDESDY |
| | | YCVLYMGSGIWVFGGGTKLTVL |
| 767 | ABL-CD3-80 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGGGTKLTVL |
| 768 | ABL-CD3-81 | NFMLTQPHSVSESPGKTVTISCTRSSGSIASNYVQWYQQRPGSS |
| | | PTTVIYEDNQRPSGVPDRFSGSIDSSSNSASLTISGLKTEDEAYY |
| | | YCQSFDSSNRVFGGGTKLTVL |
| 769 | ABL-CD3-82 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGGGTKLTVL |
| 770 | ABL-CD3-83 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSSDRSNVVFGGGTKLTVL |
| 771 | ABL-CD3-84 | NFMLTQPHSVSESPGKTVTISCTRSSGSIASNYVQWYQQRPGSS |
| | | PTTMIYEDTQRPSGVPDRFSGSIDSSSNSASLTISGLKTEDEADY |
| | | YCQSYDSSNRVFGGGTKLTVL |
| 772 | ABL-CD3-85 | NFMLTQPHSVSESPGKTVTISCTRSSGSIASNYVQWYQQRPGSS |
| | | PTTVIYEDNHRPSGVPDRFSGSIDSSSNSASLTIAGLKTEDEADY |
| | | YCQSCDSSNVVFGGGTKLTVL |
| 773 | ABL-CD3-86 | NFMLTQPLSVSESPGKTLTISCTRNSGSIASNYVQWYRQRPGSS |
| | | PTTVIYEDKQRPSGVPDRFSGSIDSSSNSASLTISGLKAEDEADY |
| | | YCQSCDSSNVVFGGGTTLTVL |
| 774 | ABL-CD3-87 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 775 | ABL-CD3-88 | DIQMTQSPSTLSASVGDRVTITCRASQSISSWLAWYQQKPGKAP |
| | | NLLIYKASSLESGVPSRFSGSGSGTEFTLTISSLQPDDFATYYCQ |
| | | QYNSYSWTFGQGTKVEIK |
| 776 | ABL-CD3-89 | EIVLTQSPGTLSLSPGERATLSCRASQSVSSSYLAWYQQKPGQA |
| | | PRLLIYGASSRATGIPDRFSGSGSGTDFTLTISRLAPEDFAVYYC |
| | | QQYGSSRWSFGQGTKVEIK |
| 777 | ABL-CD3-90 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 778 | ABL-CD3-91 | SYELTQPPSVSVSPGQTASITCSGDKLGNKYACWYQQKPGQSP |
| | | VLVIYQDSKRPSGIPERFSGSNSGNTATLTISGTQAMDEADYYC |
| | | QAWDISTVVFGGGTKLTVL |
| 779 | ABL-CD3-92 | SYELTQPPSVSVSPGQTASITCSGDKLRDKYACWYQQKPGQSP |
| | | VLVIYQDSKRPSGIPERFSGSNSGNTATLTISGTQAMDEADYYC |
| | | QAWDSSTVVFGGGTKLTVL |
| 780 | ABL-CD3-93 | SYELTQPPSVSVSPGQTASITCSGDKLGDKYAGWYQQKPGQSP |
| | | VLVIYQDSKRPSGIPERFSGSNSGNTATLTISGTQAMDEADYYC |
| | | QAWDSSTVVFGGGTKLTVL |
| 781 | ABL-CD3-94 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNHVFGGGTKLTVL |
| 782 | ABL-CD3-95 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGGGTKLTVL |
| 783 | ABL-CD3-96 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGGGTKLTVL |
| 784 | ABL-CD3-97 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGGGTKLTVL |
| 785 | ABL-CD3-98 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHCWVFGGGTKLTVL |
| 786 | ABL-CD3-99 | NFMLTQPHSVSESPGKTVTISCTRSSGSIASNYVQWYQQRPGSS |
| | | PTTMIYEDNQRPSGVPDRFSGSIDSSSNSASLTISGLKTEDEADY |
| | | YCQSLDSSSVVFGGGTKLTVL |
| 787 | ABL-CD3-100 | NFMLTQPHSVSESPGKTVTIACTRSSGSIASNYVQWYQQRPGSS |
| | | PTTVIYEDNQRPSGVPDRFSGSIDSSSNSASLTISGLKTEDEADY |
| | | YCQSYDSSNQVFGGGTKLTVL |
| 788 | ABL-CD3-101 | NFMLTQPHSVSESPGKTVTISCTRSNGSITSNYVQWYQQRPGSS |
| | | PTTVIYEDNQRPSGVPDRFSGSIDSSSNSASLTISGLKTEDEADY |
| | | YCQSFDNSNRVFGEKTKLTVL |
| 789 | ABL-CD3-102 | DIQMTQSPSTLSASVGDRVTITCRASQSISNWLAWYQQKPGKA |
| | | PNLLIYKASSLESGVPSRFSGSGSGTEFTLTISSLQPDDFANYYC |
| | | QQYKSYSWTFGQGTKVEIK |
| 790 | ABL-CD3-103 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 791 | ABL-CD3-104 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGGGTKLTVL |
| 792 | ABL-CD3-105 | DIQMTQSPSTLSASVGDRVTITCRASQSIGSWLAWYQQKPGKA |
| | | PKLLIYKASSLESGVPSRFSGSGSGTEFTLTISSLQPDDFATYYC |
| | | QQYNSYSWTFGQGTKVEIK |
| 793 | ABL-CD3-106 | NFMLTQPHSVSESPGKTVTISCTRSSGSIASKYVQWYQQRPGSS |
| | | PTTVIYEDNQRPSGVPDRFSGSIDSSSNSASLTISGLKTEDEADY |
| | | YCQSYDSNNRVFGGGTKLTVL |
| 794 | ABL-CD3-107 | DIVMTQTPLSSPVTPGQPASISCRSSQSLVHSDGNTYLNWLQQR |
| | | PGQPPRLLIYKISDRFSGVPDRFSGSGAGTDFTLKIGRVEAEDVG |
| | | VYYCMQSTQFPITFGQGTRLEIK |
| 795 | ABL-CD3-108 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGGGTKLTVL |
| 796 | ABL-CD3-109 | NFMLTQPHSVSESPGKTVTISCTRSSGSIASNYVQWYQQRPGSS |
| | | PTSVIYEDNQRPSGVPDRFSGSIDSSSNSASLTISGLKTEDEADY |
| | | YCQSYDRSNRVFGGGTKLTVL |
| 797 | ABL-CD3-110 | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTHSSGNHRVFGGGTKLTVL |
| 798 | ABL-CD3-111 | SYELTQPPSVSVSPGQTARITCSGDALPKQYTYWCQQKPGQAP |
| | | VLVIYKDSERPSGIPERFSGSSSGTTVTLTISGVQAEDEADYYCQ |
| | | SADSSGTYRVFGGGTKLTVL |
| 799 | ABL-CD3-112 | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGGGTKLTVL |
| 800 | ABL-CD3-113 | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFYSSNRVFGGGTELTVL |
| 801 | ABL-CD3-1A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 802 | ABL-CD3-2A | SYELTQPPSVSVSPGQTASITCSGDKLGNKYACWYQQKPGQSP |
| | | VLVIYQDSKRPSGIPERFSGSNSGNTATLTISGTQAMDEADYYC |
| | | QAWDISTVVFGCGTKLTVL |
| 803 | ABL-CD3-3A | SYELTQPPSVSVSPGQTASITCSGDKLRDKYACWYQQKPGQSP |
| | | VLVIYQDSKRPSGIPERFSGSNSGNTATLTISGTQAMDEADYYC |
| | | QAWDSSTVVFGCGTKLTVL |
| 804 | ABL-CD3-4A | SYELTQPPSVSVSPGQTASITCSGDKLGDKYAGWYQQKPGQSP |
| | | VLVIYQDSKRPSGIPERFSGSNSGNTATLTISGTQAMDEADYYC |
| | | QAWDSSTVVFGCGTKLTVL |
| 805 | ABL-CD3-5A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNHVFGCGTKLTVL |
| 806 | ABL-CD3-6A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGCGTKLTVL |
| 807 | ABL-CD3-7A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGCGTKLTVL |
| 808 | ABL-CD3-8A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGCGTKLTVL |
| 809 | ABL-CD3-9A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHCWVFGCGTKLTVL |
| 810 | ABL-CD3-10A | NFMLTQPHSVSESPGKTVTISCTRSSGSIASNYVQWYQQRPGSS |
| | | PTTMIYEDNQRPSGVPDRFSGSIDSSSNSASLTISGLKTEDEADY |
| | | YCQSLDSSSVVFGCGTKLTVL |
| 811 | ABL-CD3-11A | NFMLTQPHSVSESPGKTVTIACTRSSGSIASNYVQWYQQRPGSS |
| | | PTTVIYEDNQRPSGVPDRFSGSIDSSSNSASLTISGLKTEDEADY |
| | | YCQSYDSSNQVFGCGTKLTVL |
| 812 | ABL-CD3-12A | NFMLTQPHSVSESPGKTVTISCTRSNGSITSNYVQWYQQRPGSS |
| | | PTTVIYEDNQRPSGVPDRFSGSIDSSSNSASLTISGLKTEDEADY |
| | | YCQSFDNSNRVFGCKTKLTVL |
| 813 | ABL-CD3-13A | DIQMTQSPSTLSASVGDRVTITCRASQSISNWLAWYQQKPGKA |
| | | PNLLIYKASSLESGVPSRFSGSGSGTEFTLTISSLQPDDFANYYC |
| | | QQYKSYSWTFGCGTKVEIK |
| 814 | ABL-CD3-14A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 815 | ABL-CD3-15A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 816 | ABL-CD3-16A | DIQMTQSPSTLSASVGDRVTITCRASQSIGSWLAWYQQKPGKA |
| | | PKLLIYKASSLESGVPSRFSGSGSGTEFTLTISSLQPDDFATYYC |
| | | QQYNSYSWTFGCGTKVEIK |
| 817 | ABL-CD3-17A | NFMLTQPHSVSESPGKTVTISCTRSSGSIASKYVQWYQQRPGSS |
| | | PTTVIYEDNQRPSGVPDRFSGSIDSSSNSASLTISGLKTEDEADY |
| | | YCQSYDSNNRVFGCGTKLTVL |
| 818 | ABL-CD3-18A | DIVMTQTPLSSPVTPGQPASISCRSSQSLVHSDGNTYLNWLQQR |
| | | PGQPPRLLIYKISDRFSGVPDRFSGSGAGTDFTLKIGRVEAEDVG |
| | | VYYCMQSTQFPITFGCGTRLEIK |
| 819 | ABL-CD3-19A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGCGTKLTVL |
| 820 | ABL-CD3-20A | NFMLTQPHSVSESPGKTVTISCTRSSGSIASNYVQWYQQRPGSS |
| | | PTSVIYEDNQRPSGVPDRFSGSIDSSSNSASLTISGLKTEDEADY |
| | | YCQSYDRSNRVFGCGTKLTVL |
| 821 | ABL-CD3-21A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTHSSGNHRVFGCGTKLTVL |
| 822 | ABL-CD3-22A | SYELTQPPSVSVSPGQTARITCSGDALPKQYTYWYQQKPGQAP |
| | | VLVIYKDSERPSGIPERFSGSSSGTTVTLTISGVQAEDEADYYCQ |
| | | SADSSGTYRVFGCGTKLTVL |
| 823 | ABL-CD3-23A | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGCGTKLTVL |
| 824 | ABL-CD3-24A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFYSSNRVFGCGTELTVL |
| 825 | ABL-CD3-25A | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGCGTKLTVL |
| 826 | ABL-CD3-26A | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGCGTKLTVL |
| 827 | ABL-CD3-27A | QTVVTQEPSFSVSPGGTVTLTCGLNSGSVSTSYYPSWYQQTPG |
| | | QTPRTLIYSTNTRSSGVPDRFSGSILGNKAALTITGAQADDESDY |
| | | YCVLYMGSGIWVFGCGTKLTVL |
| 828 | ABL-CD3-28A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDSSNVVFGCGTKLTVL |
| 829 | ABL-CD3-29A | QSVLTQPPSASGTPGQRVTISCSGSSSNIGSNYVYWYQQLPGTA |
| | | PKLLIYSNNQRPSGVPDRFSGSKSGTSASLAISGLRSEDEADYYC |
| | | EAWDDSLSGPVFGCGTKLTVL |
| 830 | ABL-CD3-30A | NFMLTQPHSVSESPGKTVTISCTRSSGSIASNYVQWYQHRPGSS |
| | | PTTVIYEDDQRPSGIPDRFSGSIDSSSNSASLTISGLKTEDEADYY |
| | | CQSYNSSNQVFGCGTKLTVL |
| 831 | ABL-CD3-31A | DIVMTQSPLSLPVTPGEPASISCRSSQSLLHSNGYNYLDWYLQK |
| | | PGQSPQLLIYLGSNRASGVPDRFSGSGSGTDFTLKISRVEAEDV |
| | | GFYYCMQPLQTPYTFGCGTKLEIK |
| 832 | ABL-CD3-32A | SYELTQPPSVSVSPGQTASITCSGDKLGYKYASWYQQKPGQSP |
| | | VLVIYQDSKRPSGIPERFSGSNSGNTATLTISGTQAMDEADYYC |
| | | QAWDSSTVVFGCGTKLTVL |
| 833 | ABL-CD3-33A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYYNSNRVFGCGTKLTVL |
| 834 | ABL-CD3-34A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 835 | ABL-CD3-35A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHCWVFGCGTKLTVL |
| 836 | ABL-CD3-36A | DIQMTQSPSTLSASVGDRVTITCRASQSISSWLAWFQQKTGKAP |
| | | KLLIYKASSLESGVPSRFSGSGSGTEFTLTISSLQPDDFATYYCQ |
| | | QYISYSWTFGCGTKVEIK |
| 837 | ABL-CD3-37A | DIQMTQSPSTLSASVGDRVTITCRASQSISSWLAWYQQKPGKAP |
| | | KLLIHKASSLESGVPSRFSGSGSGTEFTLTISSLQPDDFATYYCQ |
| | | QYNSYWTFGCGTKVEIK |
| 838 | ABL-CD3-38A | QSVLTQPPSVSGAPGQRVTISCTGSSSNIGAGYDVHWYQQLPG |
| | | TAPKLLIYGNSNRPSGVPDRFSGSKSGTSASLAITGLQAEDETD |
| | | YNCQSYDSSLSGYVFGCGTKVTVL |
| 839 | ABL-CD3-39A | DVVMTQSPLSLPVTLGQPASISCRSSQSLVYSDGNTYLTWFQQ |
| | | RPGQSPRRLIYKVSNRDSGVPDRFSGSGSGTDFTLKISRVEAED |
| | | VEIYYCMQGTHWPLTFGCGTKVEIK |
| 840 | ABL-CD3-40A | QSVLTQPPSASGTPGQRVTISCSGSSSNIGSNTVNWYQQFPGTA |
| | | PKLLIYSNNQRPSGVPDRFSGSKSGTSASLAISGLQSEDEADYY |
| | | CAPWDDSLNGVVFGCGTKLTVL |
| 841 | ABL-CD3-41A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGCGTKLTVL |
| 842 | ABL-CD3-42A | SYELTQPPSVSVSPGQTARITCSGDALPKQYTYWCQQKPGQAPI |
| | | LVIYKDSERPSGIPERFSGSSSGTTVTLTISGVQADDEADYYCQS |
| | | ADSSGTYRVFGCGTKLTVL |
| 843 | ABL-CD3-43A | QSALTQPASVSGSPGQSITISCTGTSSDVGSYNLVSWYQQHPGK |
| | | APKLMIYEDIRRPSGVSNRFSASKSGDTASLTISGLQAEDEADY |
| | | YCSSYAGSTTFAIFGCGTKLTVL |
| 844 | ABL-CD3-44A | QPVLTQPSSHSASSGASVRLTCMLSSGFSVGDFWIRWYQQKPG |
| | | NPPRYLLFYHSDSNKGQGSGVPSRFSGSNDASANAGILRISGLQ |
| | | PEDEADYYCGTWHSNSKTWVFGCGTKLTVL |
| 845 | ABL-CD3-45A | DIQMTQSPSTLSASVGDRVTITCRASQSISSWLAWYQQKPGKAP |
| | | NLLIYKASSLESGVPSRFSGSGSGTEFTLTISSLQPDDFATYYCQ |
| | | QYKSYSWTIGCGTKVEIK |
| 846 | ABL-CD3-46A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQCYDRSNRVFGCGTKLTVL |
| 847 | ABL-CD3-47A | NFMLTQPHSVSESPGKTVTISCTRSSGSIASKYVQWYQQRPGSS |
| | | PTTVIYEDNQRPSGVPDRFSGSIDSSSNSASLTISGLKTEDEADY |
| | | YCQSYYSNNRVFGCGTKLTVL |
| 848 | ABL-CD3-48A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDSSNRVFGCGTKLTVL |
| 849 | ABL-CD3-49A | NFMLTQPHSVSESPGKTVTISCTRSSGSIASNYVQWYQQRPGSS |
| | | PTTVIYEDNQRSSGVPDRFSGSIDSSSNSASLTISGLKTEDEADY |
| | | YCQSYDRSNRVFGCGTKLTVL |
| 850 | ABL-CD3-50A | NFMLTQPHSVSESPGKTVTISCTRSSGSIASNYVQWYQQRPGISP |
| | | TTVIYEDNQRPSGVPDRFSGSIDSSSNSASLTISGLKTEDEADYY |
| | | CQSFDTSNVVFGCGTKLTVL |
| 851 | ABL-CD3-51A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 852 | ABL-CD3-52A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHRVFGCGTKLTVL |
| 853 | ABL-CD3-53A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 854 | ABL-CD3-54A | NSMLTQPHSVSESPGKTVTISCTRSSGSIASYYVQWYQQRPGSS |
| | | PTTVIYEDNQRPSGVPDRFSGSIDSSSNSASLTISGLKTEDEADY |
| | | YCQSFDSSNRVFGCGTKLTVL |
| 855 | ABL-CD3-55A | NFMLTQPHSVSESPGKTVTISCTRSSGSIASNYVQWYQQRPGSS |
| | | PTTVIYEDNQRPSGVPDRFSGSIDRSSNSASLTISGLKTEDEADY |
| | | YCQSYDSSNRVFGCGTKLTVL |
| 856 | ABL-CD3-56A | FMLTQPHSVSESPGKTVTISCTRSSGSIASNYVQWYQQRPGSSP |
| | | TTVIYEDNQRPSGVPDRFSGSIDSSSNSASLTISGLQTEDEADYY |
| | | CQSYDSSNRVFGCGTKLTVL |
| 857 | ABL-CD3-57A | QPVLTQPSSHSASSGASVRLTCMLSSGFSVGDFWIRWYQQKPG |
| | | NPPRYLLYYHSDSNKGQGSGVPSRFSGSNDASANAGILRISGLQ |
| | | PEDEADYYCGTWHSNSKTQVFGCGTKLTVL |
| 858 | ABL-CD3-58A | NFMLTQPHSVSESPGKTVTISCTRSSGSIASNFVQWYQQRPGSS |
| | | PTTVIYEDNQRPSGVPDRFSGSIDSSSNSASLTISGLKTEDEADY |
| | | YCQSFDSSNRVFGCGTKLTVL |
| 859 | ABL-CD3-59A | NFMLTQPHSVSESPGKTVTISCTRSSGSIASNYVQWYQQRPGSS |
| | | PTTVIYEDNQRPSGVPDRFSGSIDSSSNSASLTISGLQTEDEADY |
| | | YCQSYDNSIRVFGCGTKLTVL |
| 860 | ABL-CD3-60A | NFMLTQPHSVSESPGKTVTISCTRSSGSIASNYVQWYQQRPGSS |
| | | PTTVIYEDNQRPSGVPDRFSGSIDRSSNSASLTISGLKTEDEADY |
| | | YCQSYDSSNRVFGCGTKLTVL |
| 861 | ABL-CD3-61A | QTVVTQEPSLTVSPGGTVTLTCASSTGAVTSGYYPNWFQQKPG |
| | | QAPRALIYSTSNKHSWTPARFSGSLLGGKAALTLSGVQPEDEA |
| | | EYYCLLYYGGAYVFGCGTKVTVL |
| 862 | ABL-CD3-62A | NFMLTQPHSVSESPGKTVAISCTRSSGSIASNYVQWYQQRPGSS |
| | | PTAVIYEDNQRPSGVPDRFSGSIDSSSNSAFLTISGLKTEDEADY |
| | | YCQSYDSSNRVFGCGTKLTVL |
| 863 | ABL-CD3-63A | NFMLTQPHSVSESPGKTVTISCTRTSGSIASNYVQWYQQRPGSS |
| | | PTTVIYEDNQRPSGVPDRFSGSIDSSSNSASLTISGLKTEDEAAY |
| | | YCQSYDRSNRVFGCGTKLTVL |
| 864 | ABL-CD3-64A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 865 | ABL-CD3-65A | QSVLTQPPSASGTPGQRVTISCSGSSSNIGSNYVYWYQQLPGTA |
| | | PKLLIYSNNQRPSGVPDRFSGSKSGTSASLAISGLRSEDEADYYC |
| | | AAWDDSLSGVVFGCGTKLTVL |
| 866 | ABL-CD3-66A | NFMLTQPHSVSESPGKTVTISCTRSSGSIASNYVQWYQQRPGSS |
| | | PTTVIYEDNQRPSGVPDRFSGSIDSSSNSASLTISGLKTEDEGDY |
| | | YCQSYDRSNRVFGCGTKLTVL |
| 867 | ABL-CD3-67A | NFMLTQPHSVSESPGKTVTISCTRSSGSIASNYVQWYRQRPGSS |
| | | PTTVIYEDNQRPSGVPDRFSGAIDSSSTSASLTISGLKTEDEADY |
| | | YCQSYDSSNRVFGCGTKLTVL |
| 868 | ABL-CD3-68A | SYELTQPPSVSVSPGQTARITCSGDALPKQYAYWYQQKPGQAP |
| | | VLVIYKDSERPSGIPERFSGSSSGTTVTLTISGVQAEDEADYYCQ |
| | | SADSSGTYVVFGCGTKLTVL |
| 869 | ABL-CD3-69A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 870 | ABL-CD3-70A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 871 | ABL-CD3-71A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDRSNRVFGCGTKLTVL |
| 872 | ABL-CD3-72A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 873 | ABL-CD3-73A | NFMLTQPHSVSESPGKTVTISCTRSSGSIASNYVQWYQQRPGSS |
| | | PTTVIYEDNRRPSGVPDRFSGSIDSSSNSASLTISGLKTEDEADY |
| | | YCQSYDNSNRVFGCGTTLTVL |
| 874 | ABL-CD3-74A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHWVFGCGTKLTVL |
| 875 | ABL-CD3-75A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHNWVFGCGTKLTVL |
| 876 | ABL-CD3-76A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHWVFGCGTKLTVL |
| 877 | ABL-CD3-77A | QSVLTQPPSVSGAPGQRVTISCTGSSSNIGAGYDVHWYQQLPG |
| | | TAPKLLIYGNSNRPSGVPDRFSGSKSGTSASLAITGLQAEDEAD |
| | | YYCQSYDSSLSGSVFGCGTKLTVL |
| 878 | ABL-CD3-78A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDGSNRVFGCGTKLTVL |
| 879 | ABL-CD3-79A | QTVVTQEPSFSVSPGGTVTLTCGLSSGSVSTSYYPSWYQQTPGQ |
| | | APRTLIYSTNTRSSGVPDRFSGSILGNKAALTITGAQADDESDY |
| | | YCVLYMGSGIWVFGCGTKLTVL |
| 880 | ABL-CD3-80A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGCGTKLTVL |
| 881 | ABL-CD3-81A | NFMLTQPHSVSESPGKTVTISCTRSSGSIASNYVQWYQQRPGSS |
| | | PTTVIYEDNQRPSGVPDRFSGSIDSSSNSASLTISGLKTEDEAYY |
| | | YCQSFDSSNRVFGCGTKLTVL |
| 882 | ABL-CD3-82A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGCGTKLTVL |
| 883 | ABL-CD3-83A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSSDRSNVVFGCGTKLTVL |
| 884 | ABL-CD3-84A | NFMLTQPHSVSESPGKTVTISCTRSSGSIASNYVQWYQQRPGSS |
| | | PTTMIYEDTQRPSGVPDRFSGSIDSSSNSASLTISGLKTEDEADY |
| | | YCQSYDSSNRVFGCGTKLTVL |
| 885 | ABL-CD3-85A | NFMLTQPHSVSESPGKTVTISCTRSSGSIASNYVQWYQQRPGSS |
| | | PTTVIYEDNHRPSGVPDRFSGSIDSSSNSASLTIAGLKTEDEADY |
| | | YCQSYDSSNVVFGCGTKLTVL |
| 886 | ABL-CD3-85B | NFMLTQPHSVSESPGKTVTISCTRSSGSIASNYVQWYQQRPGSS |
| | | PTTVIYEDNHRPSGVPDRFSGSIDSSSNSASLTIAGLKTEDEADY |
| | | YCQSSDSSNVVFGCGTKLTVL |
| 887 | ABL-CD3-86A | NFMLTQPLSVSESPGKTLTISCTRNSGSIASNYVQWYRQRPGSS |
| | | PTTVIYEDKQRPSGVPDRFSGSIDSSSNSASLTISGLKAEDEADY |
| | | YCQSCDSSNVVFGCGTTLTVL |
| 888 | ABL-CD3-87A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 889 | ABL-CD3-88A | DIQMTQSPSTLSASVGDRVTITCRASQSISSWLAWYQQKPGKAP |
| | | NLLIYKASSLESGVPSRFSGSGSGTEFTLTISSLQPDDFATYYCQ |
| | | QYNSYSWTFGCGTKVEIK |
| 890 | ABL-CD3-89A | EIVLTQSPGTLSLSPGERATLSCRASQSVSSSYLAWYQQKPGQA |
| | | PRLLIYGASSRATGIPDRFSGSGSGTDFTLTISRLAPEDFAVYYC |
| | | QQYGSSRWSFGCGTKVEIK |
| 891 | ABL-CD3-90A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 892 | ABL-CD3-91A | SYELTQPPSVSVSPGQTASITCSGDKLGNKYACWYQQKPGQSP |
| | | VLVIYQDSKRPSGIPERFSGSNSGNTATLTISGTQAMDEADYYC |
| | | QAWDISTVVFGCGTKLTVL |
| 893 | ABL-CD3-92A | SYELTQPPSVSVSPGQTASITCSGDKLRDKYACWYQQKPGQSP |
| | | VLVIYQDSKRPSGIPERFSGSNSGNTATLTISGTQAMDEADYYC |
| | | QAWDSSTVVFGCGTKLTVL |
| 894 | ABL-CD3-93A | SYELTQPPSVSVSPGQTASITCSGDKLGDKYAGWYQQKPGQSP |
| | | VLVIYQDSKRPSGIPERFSGSNSGNTATLTISGTQAMDEADYYC |
| | | QAWDSSTVVFGCGTKLTVL |
| 895 | ABL-CD3-94A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNHVFGCGTKLTVL |
| 896 | ABL-CD3-95A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGCGTKLTVL |
| 897 | ABL-CD3-96A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGCGTKLTVL |
| 898 | ABL-CD3-97A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHSWVFGCGTKLTVL |
| 899 | ABL-CD3-98A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTDSSGNHCWVFGCGTKLTVL |
| 900 | ABL-CD3-99A | NFMLTQPHSVSESPGKTVTISCTRSSGSIASNYVQWYQQRPGSS |
| | | PTTMIYEDNQRPSGVPDRFSGSIDSSSNSASLTISGLKTEDEADY |
| | | YCQSLDSSSVVFGCGTKLTVL |
| 901 | ABL-CD3-100A | NFMLTQPHSVSESPGKTVTIACTRSSGSIASNYVQWYQQRPGSS |
| | | PTTVIYEDNQRPSGVPDRFSGSIDSSSNSASLTISGLKTEDEADY |
| | | YCQSYDSSNQVFGCGTKLTVL |
| 902 | ABL-CD3-101A | NFMLTQPHSVSESPGKTVTISCTRSNGSITSNYVQWYQQRPGSS |
| | | PTTVIYEDNQRPSGVPDRFSGSIDSSSNSASLTISGLKTEDEADY |
| | | YCQSFDNSNRVFGCKTKLTVL |
| 903 | ABL-CD3-102A | DIQMTQSPSTLSASVGDRVTITCRASQSISNWLAWYQQKPGKA |
| | | PNLLIYKASSLESGVPSRFSGSGSGTEFTLTISSLQPDDFANYYC |
| | | QQYKSYSWTFGCGTKVEIK |
| 904 | ABL-CD3-103A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 905 | ABL-CD3-104A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSYDSSNRVFGCGTKLTVL |
| 906 | ABL-CD3-105A | DIQMTQSPSTLSASVGDRVTITCRASQSIGSWLAWYQQKPGKA |
| | | PKLLIYKASSLESGVPSRFSGSGSGTEFTLTISSLQPDDFATYYC |
| | | QQYNSYSWTFGCGTKVEIK |
| 907 | ABL-CD3-106A | NFMLTQPHSVSESPGKTVTISCTRSSGSIASKYVQWYQQRPGSS |
| | | PTTVIYEDNQRPSGVPDRFSGSIDSSSNSASLTISGLKTEDEADY |
| | | YCQSYDSNNRVFGCGTKLTVL |
| 908 | ABL-CD3-107A | DIVMTQTPLSSPVTPGQPASISCRSSQSLVHSDGNTYLNWLQQR |
| | | PGQPPRLLIYKISDRFSGVPDRFSGSGAGTDFTLKIGRVEAEDVG |
| | | VYYCMQSTQFPITFGCGTRLEIK |
| 909 | ABL-CD3-108A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFDNSNRVFGCGTKLTVL |
| 910 | ABL-CD3-109A | NFMLTQPHSVSESPGKTVTISCTRSSGSIASNYVQWYQQRPGSS |
| | | PTSVIYEDNQRPSGVPDRFSGSIDSSSNSASLTISGLKTEDEADY |
| | | YCQSYDRSNRVFGCGTKLTVL |
| 911 | ABL-CD3-110A | SYELTQPPSVSVSPGQTARITCSGDALPKKYAYWYQQKSGQAP |
| | | VLVIYEDSKRPSGIPERFSGSSSGTMATLTISGAQVEDEADYYC |
| | | YSTHSSGNHRVFGCGTKLTVL |
| 912 | ABL-CD3-111A | SYELTQPPSVSVSPGQTARITCSGDALPKQYTYWCQQKPGQAP |
| | | VLVIYKDSERPSGIPERFSGSSSGTTVTLTISGVQAEDEADYYCQ |
| | | SADSSGTYRVFGCGTKLTVL |
| 913 | ABL-CD3-112A | NFMLTQPHSVSESPGKTVTISCTRSSGSIASNYVQWYQQRPGSS |
| | | PTTVIYEDNHRPSGVPDRFSGSIDSSSNSASLTISGLKTEDEADY |
| | | FCQSFDSSNRVFGCGTKLTVL |
| 914 | ABL-CD3-113A | NFMLTQPHSVSESPGKTVTISCTRSSGSIASNYVQWYQQRPGSS |
| | | PTTVIYEDNQRPSGVPDRFSGSIDSSSNSASLTISGLKTEDEADY |
| | | YCQSFYSSNRVFGCGTELTVL |

TABLE 1E
VH Sequences

| | | |
|---|---|---|
| SEQ | | |
| ID | | |
| NO: | Name | Sequence |

Heavy Chain Variable Region Sequences

| | | |
|---|---|---|
| 915 | ABL-CD3-1 | EVQLVESGGGLVKPGGSLRLSCAASGFTLSSYSMNWVRQAPG |
| | | KGLEWVSSISRSGSYIYYADSVKGRFTISRDNAKNSLYLQMNS |
| | | LRAEDTAVYYCAREKWELLYFDYWGQGTLVTVSS |
| 916 | ABL-CD3-2 | EVQLVESGGGLVKPGGSLRLSCAASGFTFSSYSMNWVRQAPG |
| | | KGLEWVSSISRSTSYIYYADSVKGRFTISRDNAKNSLYLQMNSL |
| | | RAEDTAVYYCARGSRDAFDIWGQGTMVTVSS |
| 917 | ABL-CD3-3 | EVQLVESGGGLVKPGGSLRLSCAASGFTFSSYSMNWVRQAPG |
| | | KGLEWVSYISRSSNYIHYADSVKGRFTIFRDNAKNSLYLQMNS |
| | | LRVEDTAVYYCARAERGSYAVDIWGQGTMVTVSS |
| 918 | ABL-CD3-4 | VQLVESGGGLVKPGGSLRLSCAASGFTFSSYNMNWVRQAPGK |
| | | GLEWVSSISSSSSYIYYADSVKGRFTISRDNAKNSLYLQMNSLR |
| | | AEDTAVFYCARAERGSYAFDIWGQGTMVTVSS |
| 919 | ABL-CD3-5 | EVQLVESGGGLVQPGGSLRLSCAASGFTFSRYSMNWVRQAPG |
| | | KGLEWVSYISSSSSSIYYADSVKGRFTISRDNAKNSLYLQMNSL |
| | | RDEDTAVYYCARDRRATLDYWGQGTLVTVSS |
| 920 | ABL-CD3-6 | EVQLVDSGGGLVKPGGSLRLSCAASGFTFSSYSMNWVRQAPG |
| | | KGLEWVSSISSRGSYRNYADSVKGRFTISRDNAKNSLYLQMNS |
| | | LRAEDTAVYYCARDRRELLLDCWGQGTLVTVSS |
| 921 | ABL-CD3-7 | EVQLVESGGGLVKPGGSLRLSCAASGFTFSRYSMNWVRQAPG |
| | | KGLKWVSSISSSGSYKYYADSVKGRFTISRDNAKNSLYLQMNS |
| | | LRAEDTAVYYCAREPPTLGMDYWGQGTLVTVSS |
| 922 | ABL-CD3-8 | EVQLVESGGGLVKPGGSLRLSCAASGFTFSSYSMNWVRQAPG |
| | | KGLEWVSSISSSSSYIYYADSVKGRFTISRANAKNSLYLQMSSL |
| | | RAEDTAVYYCARDRRELLLDYWGQGTLVTVSS |
| 923 | ABL-CD3-9 | EVQLVDSGGGLVKPGGSLRLSCAASGFTFSSYSMNWVRQAPG |
| | | KGLEWVSSVNSRGSYIYYADSVKGRFTISRDNAKNSLYLQMNS |
| | | LRADDTAVYYCARDRRELLLDYWGQGTLVTVSS |
| 924 | ABL-CD3-10 | QVQLQQSGPGLVKPSQTLSLTCAISGDSVSCNSVAWNWIRQSP |
| | | SRGLEWLGRTYYRSKWHNEYAVSVKSRINIKPDTSKNQVSLQL |
| | | KSVTPEDTAVYYCARERKWELFLDYWGQGTLVTVSS |
| 925 | ABL-CD3-11 | QVTLKESGPGILQPSQTLSLTCSFSGFSLSTFGMGVGWIRQPSG |
| | | KGLEWLAHIWWDDDKYYNPALKSRLTISKDTSKNQVFLKIAN |
| | | VDTADTATYYCARLGYYPYWFFDVWGTGTTVTVSS |
| 926 | ABL-CD3-12 | EVQLVESGGGLVKPGGSLRLSCAASGFTFSSYTMNWVRQAPG |
| | | KGLEWVSSISRSSSYKYYADSVKGRFTISRDNAKNSLYLQMNS |
| | | LRAEDTAVYYCAREPPTLGMDYWGQGTLVIVSS |
| 927 | ABL-CD3-13 | EVQLVESGGGLVQPGGSLRLSCAASGFTFSNYWMSWVRQAPG |
| | | KGLEWVANIKEDGSEKYYVDSVKGRFTISRDNAKNSLYLQMN |
| | | SLTAEDTSVYYCARERRTGYFDYWGQGTLVTVSS |
| 928 | ABL-CD3-14 | EVQLVESGGGLVKPGGSLRLSCAASGFTCSSYSMTWVRQAPG |
| | | KGLEWVSSISRRSSYIYYADSVKGRFTISRDNAKNSLYLQLNSL |
| | | RAEDTAVYYCARERWELLYFDYWGQGTLVTVSS |
| 929 | ABL-CD3-15 | EVQLVESGGGLVKPGGSLRLSCAASGFNCSSYSMNWVRQAPG |
| | | KGLEWVSSISRSSNYIYYADSVKGRFSISRDDAKNSLYLQMNSL |
| | | RAEDTAVYYCARERWIQLYSDYWGQGILVTVSS |
| 930 | ABL-CD3-16 | EVQLVESGGGLVQPGGSLRLSCAASGFTFSRYWMTWVRQAPG |
| | | KGLEWVANIKEDGSEKYCVDSVKGRFTISRDNAKNSLYLQLNS |
| | | LRAEDTAVYYCARERRTGYFDYWGQGTLVTVSS |
| 931 | ABL-CD3-17 | EVQLVESGGGLVQPGGSLRLSCAASGFTFSRYSINWVRQAPGK |
| | | GLEWVSYISSSSSTIYYADSVKGRFTISRDNAKNSLYLQMNSLR |
| | | DEDTAVYYCARERWELLYFDYWGQGTLVTVSS |
| 932 | ABL-CD3-18 | EVQLVESGEGLVQPGGSLRLSCAASGFTFSSYAMHWVRQAPG |
| | | KGLEYVSAISSNGGRTYYADSVKGRFTISRDNSKNTLYLQMGS |
| | | LRAEDMAVYYCARGGSLDYWGQGTLVTVSS |
| 933 | ABL-CD3-19 | EVQLVESGGGLVKPGGSLRLSCAASGFTLSSYSMNWVRQAPG |
| | | KGLEWVSSISRSSSYIYYADSVKGRFTISRDNAKNSLYLQMNSL |
| | | RAEDTAVYYCARERWELLYFDYWGQGTLVTVSS |
| 934 | ABL-CD3-20 | EVQLVESGGGLVKPGGSLRLSCAASGLTFSTYSMNWVRQAPG |
| | | KGLEWVSSISSSGSYVYYADSVKGRFTMSRDNAKNSLYLQMN |
| | | SLRAEDTAVYYCARERRELLFDYWGQGTLVTVSS |
| 935 | ABL-CD3-21 | EVQLVESGGGLVKPGGSLRLSCAASGFTFSSYSMNWVRQAPG |
| | | KGLEWVSSISRSSSYIYYADSVKGRFTLSRDNAKNSLYLQMNS |
| | | LRGEDTAIYYCGRDGKGAIFLDYWGQGTLVTVSS |
| 936 | ABL-CD3-22 | EVQLVESGGGLVQPGGSLRLSCAASGFTFSSYSMNWVRQAPG |
| | | KGLEWVSYISSSSSTIYYADSVKGRFTISRDNAKNSLYLQMNSL |
| | | RDEDTAVYYCARDGPVDYGMDVWGQGTTVTVSS |
| 937 | ABL-CD3-23 | EVHLVESGGGLVKPGGSLRLSCAASGFTFSSYSMNWVRQAPG |
| | | KGLEWVSSISSSSTYIYYADSVKGRFTISRDNAKNSLYLQMNSL |
| | | RAEDTAVYYCAREPRTLGMDYWGQGILVTVSS |
| 938 | ABL-CD3-24 | EVQLVESGGGLVKPGGSLRLSCVASGFTFSSYTMNWVRQAPG |
| | | KGLEWVSSISRSGSYKYNADSVKGRFTISRDNAKNSLYLQMNS |
| | | LRVEDTAVYYCAREPPTLGMDYWGQGTLVTVSS |
| 939 | ABL-CD3-25 | EVHLVESGGGLVKPGGSLRLSCAASGFTFSSYSMNWVRQAPG |
| | | KGLEWVSSISSSSTYIYYADSVKGRFTISRDNAKNSLYLQMNSL |
| | | RAEDTAVYYCAREPRTLGMDYWGQGILVTVSS |
| 940 | ABL-CD3-26 | EVHLVESGGGLVKPGGSLRLSCAASGFTFSSYSMNWVRQAPG |
| | | KGLEWVSSISSSSTYIYYADSVKGRFTISRDNAKNSLYLQMNSL |
| | | RAEDTAVYYCAREPRTLGMDYWGQGILVTVSS |
| 941 | ABL-CD3-27 | QVQLVQSGAEVKKPGASLKVSCKASGYTFTSYYMHWVRQAP |
| | | GQGLEWMGIINPSNGNTISAQKFQGRVTMTRDTSTSTVYMEM |
| | | SSLRSEDTAVYYCVRCWGYYVMDVWGQGTTVTVSS |
| 942 | ABL-CD3-28 | QVQLQQSGPGLVKPSQTLSLTCAISGDSVSSNSVAWNWIRQSP |
| | | SRGLEWLGRTYYRSKWYNECAGSVKSRITINPDTSKNQFSLQL |
| | | NSVTPEDTAIYYCARERKWELFLDYWGQGTLVTVSS |
| 943 | ABL-CD3-29 | EVQLVQSGAEVKKPGESLKISCKGSGYSFISYWIAWVRQMPGK |
| | | GLEWMGIIYPGDSETRYRPSFQGQVTISADKSINTAYLQWSSLK |
| | | AADTAMYYCARQGAYDGFDIWGQGTMVTVSS |
| 944 | ABL-CD3-30 | QVQLQESGPGLVKPSETLSLTCTVSGGSINSYYWSWIRQSPGK |
| | | GLEWIGHIYYSGRTKYNPSLKSRVTISVDTSKNQFSLKLSSVTA |
| | | ADTAVYYSARENRNGAFDIWGQGTVVTVSS |
| 945 | ABL-CD3-31 | EVQLVQSGAEVKKPGESLKISCKGSGYSFTNYWIGWVRQMPG |
| | | KGLEWMGIIYPADSDTTYSPSFQGQVTISADKSITTAYLQWSSL |
| | | KASDTAMYYCARHGGSYILDAFDIWGQGTMVTVSS |
| 946 | ABL-CD3-32 | EVQLVESGGGLVKPGGSLRLSCEASGFTFSSYNMNWVRQAPG |
| | | KGLEWVSSISSSSSYIYYTDSVKGRFTISRDNAKNSLYLQMNSL |
| | | RAEDTAVYYCARAERGSYAFDFWGQGTMVTVSS |
| 947 | ABL-CD3-33 | EVQLVESGGGLVKPGGSLRLSCAASGFTFSSYSMNWVRQAPG |
| | | KGLEWVSSISRSSSYIYYADSVKGRFTISRDNAKNSLYLQMNSL |
| | | RAEDTAVYYCAREPPTLGMDYWGQGTLVTVSS |
| 948 | ABL-CD3-34 | EVHLVESGGGLVKPGGSLRLSCAASGFTFSRYSMNWVRQAPG |
| | | KGLEWVSSISRSSSYIYYADSVKGRFTISRDNAKNSLYLQMNSL |
| | | RAEDTAVYYCAREPRTLGMDYWGQGILVTVSS |
| 949 | ABL-CD3-35 | EVQLVESGGGLVKPGGSLRLSCAASGFTFSSYSMNWVRQAPG |
| | | KGLEWVSSVNSRGSYIYYADSVKGRFTISRDNAKDSLYLQMNS |
| | | LRADDTAVYYCARDRRELLLDYWGQGTLVTVSS |
| 950 | ABL-CD3-36 | EVQLVESGGGLVQRGGSLRLSCAASGFTFSNYWMSWVRQAPG |
| | | KGLEWVANIKEDGSEKYCVDSVKGRFTISRDNAKHSLYLQMN |
| | | SLRAEDTAVYYCARERRTGYFDYWGQGTLVTVSS |
| 951 | ABL-CD3-37 | EVQLVESGGGLVQPGGSLRLSCAASGFSFSSYSMNWVRQAPG |
| | | KGLEWVSHIRSSTYSIYYADSVMGRFTISRDNAKNSLYLQMNS |
| | | LRDEDTAVYFCARAKTGGDAFDIWGQGTMVTVSS |
| 952 | ABL-CD3-38 | QGQLQQWGAGLLKPSETLSLTCAVYGGSFSGYYWSWIRLPPG |
| | | KGLEWIGEINHSGNTNYNPSLKSRVTISVDTSKNQFSLQLSSVT |
| | | AADTAVYYCAKEVEIWGYFDYWGQGTLVTVSS |
| 953 | ABL-CD3-39 | EVQLLESGGGLVQPGGSLRLSCAASGFTFSYYAMSWVRQAPG |
| | | KGLEWVSAISGSGGSTYYADSVKGRFTISRDNSKNTLYLQMNS |
| | | LRAEDTAVYYCAKEGIVGARAEYFQHWGQGTLVTVSS |
| 954 | ABL-CD3-40 | EVQLLESGGGLVQPGGSLRLSCAASGFTFSYYAMSWVRQAPG |
| | | KGLEWVSAISGSGGSTYYADSVKGRFTISRDNSKNTLYLQMNS |
| | | LRAEDTAVYYCAKEGIVGARAEYFQHWGQGTLVTVSS |
| 955 | ABL-CD3-41 | EVQLVESGGGLVKPGGSLRLSCAASGFTFSSYSMNWVRQAPG |
| | | KGLEWVSSVNSRGSYIYYADSVKGRFTISRDNAKDSLYLQMNS |
| | | LRADDTAVYYCARDRRELLLDYWGQGTLVTVSS |
| 956 | ABL-CD3-42 | EVQLVESGGGLVQPGGSLRLSCAASGFTFKSYSINWVRQAPGK |
| | | GLEWVSYISSSSSTIYYADSVKGRFTISRDNAKNSLYLQMNSLR |
| | | DEDTAVYYCARDGPVDYGMDVWGQGTTVTVSS |
| 957 | ABL-CD3-43 | EVQLVQSGAEVKKPGESLKISCKGSEYSFTTYWIAWVRQMPG |
| | | KGLEWMGIIYPGDSDTTYSPSFQGQVTISADKSISIAFLQWSSLK |
| | | ASDTAMYYCARQGELDAFDIWGQGTMVTVSS |
| 958 | ABL-CD3-44 | EVQLVESGGGLVQPGGSLRLSCAASGFTFSTYWMSWVRQAPG |
| | | KGLEWVANIKQDGSEKYYVDSVKGRFTISRDNAKNSLYLQMN |
| | | SLRAEDTAVYYCATGSFDYWGQGTLVTVSS |
| 959 | ABL-CD3-45 | EVQLVESGGGLVQPGGSLRLSCAASGFTFSSYWMSWVRQAPG |
| | | KGLEWVANIKEDGSEKYYVDSVKGRFTISRDNAKNSLYLQMN |
| | | SLRAEDTAVYYCARERRTGYLDYWGQGTLVTVSS |
| 960 | ABL-CD3-46 | EVQLVESGGGLVKPGGSLRLSCSASGFTFSTYTMNWVRQAPG |
| | | KGLDWVSSISKSSTYIYYADSVKGRFTISRDNAKNSLYLQMNS |
| | | LRAEDTAVYYCARERRTLAFDIWGQGTMVTVSS |
| 961 | ABL-CD3-47 | EVQLVESGGGLVQPGGSLRLSCAASGFTFSRYSMNWVRQAPG |
| | | KGLEWVSYISRSSTTIYYADSVKGRFTISRDNAKNSLYLQMNSL |
| | | RDEDAAVYYCARERWELLYFDYWGQGTLVTVSS |
| 962 | ABL-CD3-48 | EVQLVESGGGLVKPGGSLRLSCAASGFTFSSYTMNWVRQAPG |
| | | KGLEWVSSISGSGSYIYYADSVKGRFTISRDNAKNSLYLQMNS |
| | | LRAEDTAVYYCAREPPTLGMDYWGQGTLVTVSS |
| 963 | ABL-CD3-49 | EVQLVESGGGLVKPGGSLRLSCSASGFTFSTYTMNWVRQAPG |
| | | KGLEWVSSISKSSSYIYYTDSVKGRFTISRDNAKNSLYLQMNSL |
| | | RAEDTAVYYCARERRTLAFDIWGQGTMVTVSS |
| 964 | ABL-CD3-50 | QVQLQQSGPGLVKPSQTLSLTCAISGDSVSSNSVAWNWIRQSP |
| | | SRGLEWLGRTYSRSKWYNDYAVSVKSRITINPDTSKNQFSLEL |
| | | NSVTPEDTAVYYCARERKWELFLDYWGQGTLVTVSS |
| 965 | ABL-CD3-51 | EVQLVESGGGLVKPGGSLRLSCAASGFTFSSYSMNWVRQAPG |
| | | KGLEWVSSISRSSSYIYYADSVKGRFTISRDNAKNSLYLQMNSL |
| | | RAEDTAVYYCARERWELLYFDYWGQGTLVTVSS |
| 966 | ABL-CD3-52 | EVQLVESGGGLVKPGGSLRLSCAASGFTFSYYSMNWVRQAPG |
| | | KGLEWVSSISRSTTYIYYADSVKGRFTISRDNAKNSLYLQMNSL |
| | | RAEDTAVYYCARDGKGAIFLDYWGQGTLVTVSS |
| 967 | ABL-CD3-53 | EVQLVESGGGLVKPGGSLRLSCAASGFTFSSYTMNWVRQAPG |
| | | KGLEWVSSITTSSSYIYYADSVKGRFTISRDNAKNSLYLQMNSL |
| | | RAEDTAVYYCARVTFDAFDIWGQGTMVTVSS |
| 968 | ABL-CD3-54 | EVQLVESGGGLVKPGGSLRLSCAASGFTFSSYSMNWVRQAPG |
| | | KGLEWVSSISRSSRYIFYADSVRGRFTISRDNAKNSLYLQMNSL |
| | | RAEDTAVYYCAREKRELLFDYWGQGTLVTVSS |
| 969 | ABL-CD3-55 | EVQLVESGGGLVKPGGSLRLSCAASGFTFSIYSMNWVRQAPGK |
| | | GLEWVSSISSGSSYIYYADSVKGRFTISRDNAKSSLFLQMNSLR |
| | | AEDTAVYYCAREKRELLFDYWGQGTLVTVSS |
| 970 | ABL-CD3-56 | EVQLVESGGGLVKPGGSLRLSCVASGFTFSRYSMNWVRQAPG |
| | | KGLEWVSSISSSSRYTYYADSLKGRFIMSRDNAKNSLFLQMNS |
| | | LRAEDTAVYYCARNLYNALDYWGQGTLVTVSS |
| 971 | ABL-CD3-57 | EVQLVESGGGLVKPGGSLRLSCAASGFTFSSYTMNWVRQAPG |
| | | KGLEWVSSISTSSRYIYYTDSVKGRFTISRDDAKNSLYLQMNSL |
| | | RAEDTAVYYCTRNWGYDVFDIWGQGTMVIVSS |
| 972 | ABL-CD3-58 | EVQLVESGGGLVKPGGSLRLSCVVSGFTFSNYNMNWVRQAPG |
| | | KGLEWVSSSSRSSYIYYADSVKGRFTISRDNAKNSLYLQMNSL |
| | | RAEDTAIYYCAREKWELLFFDYWGQGTLVTVSS |
| 973 | ABL-CD3-59 | EVQLVESGGGLVKPGGSLRLSCTASGFTLSSYSMNWVRQAPG |
| | | KGLEWVSSLSRSSNYIYYAVSVKGRFTISRDNAKNSLYLQMNS |
| | | LRAEDTAVYYCARERWELLYFDYWGQGTLVTVSS |
| 974 | ABL-CD3-60 | EVQLVESGGGLVKPGGSLRLSCAASGFTFSIYSMNWVRQAPGK |
| | | GLEWVSSISSGSSYIYYADSVKGRFTISRDNAKSSLFLQMNSLR |
| | | AEDTAVYYCAREKRELLFDYWGQGTLVTVSS |
| 975 | ABL-CD3-61 | EVQLVESGGGLVKPGGSLRLSCAASGFTFSSYSMNWVRQAPG |
| | | KGLEWVSSISRSGTYIYYADSVKGRFTISRDNAKNSLYLQMNS |
| | | LRAEDTAVYYCARERWELLYFDYWGQGTLVTVSS |
| 976 | ABL-CD3-62 | EVQLVESGGGLVKPGGSLRLSCAASGFTFSSYSMNWVRQAPG |
| | | KGLEWVSSISRSSGYIFYADSVKGRFTISRDNAKNSLYLQMNSL |
| | | RAEDTAVYYCVRERVGAGFDYWGQGTLVTVSS |
| 977 | ABL-CD3-63 | QVQLQESGPGLVKPSETLSLTCTVSGGSISSYYWSWIRQPAGK |
| | | GLEWIGRIYTSGSTKYNPSLKSRVTMSVDTSKNQFSLKLISVTA |
| | | ADTAVYFCARERIRDLYFDYWGQGTLVTVSS |
| 978 | ABL-CD3-64 | EVQVAESGGGLVKPGGSLRLSCAASGFTFSRYSMNWVRQAPG |
| | | KGLEWVSSISSSGSYTYYADSVKGRFTISRDNAKNSLYLQMNS |
| | | LRAEDTAVYYCVRERGKGAGMDVWGQGTTVTVSS |
| 979 | ABL-CD3-65 | QVQLQQWGAGLLKPSETLSLTCAVYGGSFSGYYWSWIRQPPG |
| | | KGLEWIGEINHSGSTNYNPSLKSRVTISVDTSKNQFSLKLSSVT |
| | | AADTAVYYCAREYYYYGMDVWGQGTTVTVSS |
| 980 | ABL-CD3-66 | QVQLQESGPGLVKPSETLSLTCTVSGGSISSYYWSWIRQPAGK |
| | | GLEWIGRIYASGSTYYNPSLKSRVTMSVDTSKNQFSLKLSSVTA |
| | | ADTAVYYCAREKRSRLYFDYWGQGTLVTVSS |
| 981 | ABL-CD3-67 | EVQLVESGGGLVKPGGSLRLSCAASGFTVSSYSMNWVRQAPG |
| | | KGLEWVSSISRSSSYIYYADSVKGRFTISRDNAKNSLFLQMNSL |
| | | RAEDTAVYYCARERWELLYFDYWGQGTLVTVSS |
| 982 | ABL-CD3-68 | EVQLVESGGGLVKPGGSLRLSCAASGFTFSSSIMNWVRQAPGK |
| | | GLEWVSSISGSSSYIYYADSVKGRFTISRDNAKNSQYLQMKSLR |
| | | AEDTAVYYCARERWNLLYFDYWGQGTLVTVSS |
| 983 | ABL-CD3-69 | EVQLVESGGGLVKPGGSLRLSCAASGFTFSTYTMNWVRQAPG |
| | | KGLEWVSTITSSSRYIYYADSVKGRFTISRDNAKNSLYLQMNSL |
| | | RAEDTAVYYCARVTFDAFDIWGQGTMVTVSS |
| 984 | ABL-CD3-70 | EVQLVESGGGLVKPGGSLRLSCAASGFTFSTYIMNWVRQAPG |
| | | KGLEWVSSITSSSSYIYYADSVKGRFTISRDNAKNSLYLQMNSL |
| | | RAEDTAVYYCAREKWELLFFDYWGQGTLVTVSS |
| 985 | ABL-CD3-71 | QVQLQESGPGLVKPSETLSLTCTVSGGSINSYYWSWIRQPAGK |
| | | GLEWIGRIYTSGSTNYNPSLKSRVTMSVDTSKKQFSLKLSSVTA |
| | | ADTAVYYCARERISRLYFDYWGQGILVTVSS |
| 986 | ABL-CD3-72 | EVQLVESGGGLVKPGGSLRLSCAASGFTFSTYIMNWVRQAPG |
| | | KGLEWVSSISSSRSYIYYADSVKGRFTISRDNAKNSLYLQMNSL |
| | | RAEDTAVYYCAREDRYRLFFDYWGQGTLVTVSS |
| 987 | ABL-CD3-73 | EVQLVESGGGLVKPGGSLRLSCAASGFTITSYSMNWVRQAPG |
| | | KGLEWVSTFSRSSTYIYYADSVKGRFTISRDNAKNSLYLQMNS |
| | | LRAEDTAVYYCAREKWDLLYFDYWGQGTLVTVSS |
| 988 | ABL-CD3-74 | EVQLVESGGGLVKPGGSLRLSCAASGFTFSSYSMKWVRQAPG |
| | | KGLEWVSSISSSSSYIYYADSVKGRFTISRDNAKNSLYLQMNSL |
| | | RAEDTAVYYCARDRGELTLWYWGQGTLVTVSS |
| 989 | ABL-CD3-75 | EVQLVESGGGLVKPGGSLRLSCAASGFTFSTYSLNWVRQAPGK |
| | | GLEWVSSISVSSRYIYFADSVKGRFTISRDNAKNSLYLQMNSLR |
| | | AEDTAVYYCARDRRELLLDYWGQGTLVTVSS |
| 990 | ABL-CD3-76 | EVQLVESGGGLVKPGGSLRLSCAASGFTFSNYYMNWVRQAPG |
| | | KGLERVSSISSSSNYIYYADSVKGRFTISRDNAKNSLYLQMNSL |
| | | RAEDTAVYYCARDRRELPLDYWGQGTVVTVSS |
| 991 | ABL-CD3-77 | EVQLVESGGGLVKPGGSLRLSCAASGFTFSSYSMNWVRQAPG |
| | | KGLEWVSSISSSSSYIYYADSVKGRFTISRDNAKNSLYLQMNSL |
| | | RAEDTAVYYCARDGELGIGTYFDYWGQGTLVTVSS |
| 992 | ABL-CD3-78 | EVQLVESGGGLVKPGGSLRLSCAASGFTVSNYSMNWVRQAPG |
| | | KGLEWVSCISRSSSYIYYADSVKGRFTISRDNAKNSLFLQMNSL |
| | | RAEDTAVYYCARERWELLYFDYWGQGTLVTVSS |
| 993 | ABL-CD3-79 | EVQLVESGGGLVKPGGSLRLSCAASGFTVSNYSMNWVRQAPG |
| | | KGLEWVSCISRSSSYIYYADSVKGRFTISRDNAKNSLFLQMNSL |
| | | RAEDTAVYYCARERWELLYFDYWGQGTLVTVSS |
| 994 | ABL-CD3-80 | EVQLVESGGGLVKPGGSLRLSCAASGFTFSGYSMNWVRQAPG |
| | | KGLEWVSSISSRSRYIYYADSVQGRFTISRDNSKNSLFLQMNSL |
| | | RAEDTAVYYCGRDWRELLLDYWGQGTLVTVSS |
| 995 | ABL-CD3-81 | QVQLQESGPGLVKPSQTLSLTCTVSGGSISSGGYYWSWIRQHP |
| | | GKGLEWIGYIYYSGSTYYNPSLKSRVTISVDTSKDQFSLKLSSV |
| | | TAADTAVYYCARERKTSLPFDIWGQGTMVTVSS |
| 996 | ABL-CD3-82 | EVQLVESGGGLVKPGGSLRLSCAASGFTFSSYSMNWVRQAPG |
| | | KGLEWVSSISSSSSYIYYADSVKGRFTISRDNAKNSLYLQMNSL |
| | | RAEDTAVYYCARDRRELLLDYWGQGTLVTVSS |
| 997 | ABL-CD3-83 | EVQLVESGGGLVKPGGSLRLSCAASGFTFSSYSMNWVRQAPG |
| | | KGLEWVSTISRSGGYIYDADSVKGRFTISRDNAKNSLYLQMNS |
| | | LRAEDTAVYYCARDFNWTFDNWGQGTLVTVSS |
| 998 | ABL-CD3-84 | EVQLVESGGGLVKPGGSLRLSCAASGFTFSNFIMNWVRQAPGK |
| | | GLEWVSSISGSSNYIYYADSVKGRFTISGDNAKNSLYLQMNSL |
| | | RAEDTAVYYCAREKWVLLFFDYWGQGTLVTVSS |
| 999 | ABL-CD3-85 | EVQLVESGGGLVKPGGSLRLSCAASGFTFSSYSMNWVRQAPG |
| | | KGLEWVSSISRRSGYIYYADSVKGRFTLSRDNAKKSLYLQMNS |
| | | LRAEDTAVYYCARDLNWAFDYWGQGTLVTVSS |
| 1000 | ABL-CD3-86 | EVQLVESGGGLVKPGGSLRLSCAASGFTFSRYSMNWVRQAPG |
| | | KGLEWVSTISRSSRYIYYGDSVKGRFTISRDNAKNSLLLQMNSL |
| | | RAEDTAVYYCARDHNWSFDYWGKGILVTVSS |
| 1001 | ABL-CD3-87 | EVQLVESGGGLVKFGGSLRLSCAASGFTFSRYSMNWVRQAPG |
| | | KGLEWVSSISSSSSYKYYADSVKGRFTISRDNAKNSLYLQMNS |
| | | LRAEDTAVYYCAREGGNSHGMDVWGQGTTVTVSS |
| 1002 | ABL-CD3-88 | EVQLVESGGGLVQPGGSLRLSCAASGFTFSRYWMSWVRQAPG |
| | | KGLEWVANIKEDGSEKYYVDSVKGRFTISRDNAKNSLYLQMN |
| | | SLRAEDTAVYFCARERRTGYFDYWGQGTLVTVSS |
| 1003 | ABL-CD3-89 | EVQLVESGGGLVQPGGSLRLSCAASGFTFSSYWMSWVRQAPG |
| | | KGLEWVSSISSRRGYKYHADSVKGRFTISRDNAKNSLYLQMNS |
| | | LRAEDTAVYYCARVPYSGSYFDYWGQGTLVTVSS |
| 1004 | ABL-CD3-90 | EVQLVESGGGLVKPGGSLRLSCAASGFTFSSYSMNWVRQAPG |
| | | KGLEWVSSISSSSSYIYYADSVKGRFTISRDNAKNSLYLQMNSL |
| | | RAEDTAVYYCTNYRYNWNDYWGQGTLVTVSS |
| 1005 | ABL-CD3-91 | EVQLVESGGGLVKPGGSLRLSCAASGFTFSYYSMNWVRQAPG |
| | | KGLEWVSSISSSSTYIYYADSVKGRFTISRDNAKNSLYLQMNSL |
| | | RAEDTAVYYCANYRYNWNDYWGQGTLVTVSS |
| 1006 | ABL-CD3-92 | EVQLVESGGGLVKPGGSLRLSCAASGFTFSSYSMNWVRQAPG |
| | | KGLEWVSYISSSSRYIYYADSVKGRFTISRDNAKNSLYLQMNS |
| | | LRAEDTAVYYCAREGPNWGTHAFDIWGQGTMVTVSS |
| 1007 | ABL-CD3-93 | EVQLVESGGALVKPGGSLRLSCAASGFTFSRYSMNWVRQAPG |
| | | KGLEWVSSISSSSSYIYYADSVKGRFTISRDNAKNSLYLQMNSL |
| | | RAEDTAVYYCANYRYNWNDYWGQGTLVAVSS |
| 1008 | ABL-CD3-94 | EVQLVESGGGLVKPGGSLRLSCSASGFTFSSYSMNWVRQAPG |
| | | KGLEWVSSISRSSSYIFYADSVKGRFTISRDNAKNSLYLQMNSL |
| | | RAEDTAVYYCARERRNGFDYWGQGTLVTVSS |
| 1009 | ABL-CD3-95 | EVQLVESGGGLVKPGGSLRLSCAASGFTFSRYSMNWVRQAPG |
| | | KGLEWVSSISRSSSYIYYADSVKGRFTISRDNAKNSLYLQMNSL |
| | | RAEDTAVYYCANYRYNWNDYWGQGTLVTVSS |
| 1010 | ABL-CD3-96 | EVQLVESGGGLVKPGGSLRLSCAASGFTFSRYSMNWVRQAPG |
| | | KGLEWVSSISSSSSYIYYADSVKGRFTISRDNAKNSLYLQMNSL |
| | | RAEDTAVYYCARAVYNYFDQWGQGTLVTVSS |
| 1011 | ABL-CD3-97 | EVQLVESGGGLVKPGGSLRLSCAASGFTFSSYSMNWVRQAPG |
| | | KGLEWVSYISRSSTYIYYADSMKGRFTISRDNAKNSLYLQMNS |
| | | LRAEDTAVYYCAREGPNWGTHAFDIWGQGTMVTVSS |
| 1012 | ABL-CD3-98 | EVQLVESGGGLVKPGGSLRLSCAASGLTFSSYSMNWVRQAPG |
| | | KGLEWVSSISSSSSYIYYADSVKGRFTISRDNAKNSLYLQMNSL |
| | | RAEDTAVYYCARERRELAFDIWGQGTMVTVSS |
| 1013 | ABL-CD3-99 | EVQLVESGGGLVKPGGSLRLSCAASGITFSSYSMNWVRQAPGK |
| | | GLEWVSSISSSSSYIYYADSVKGRFTISRDNAKNSLHLQMNSLR |
| | | AEDTAVYYCARERRELSFDYWGQGTLVTVSS |
| 1014 | ABL-CD3-100 | EVQLVESGRGLVNPGGSLRLSCAASGFTFSSYSMNWVRQAPG |
| | | KGLEWVSYISSSSNYIYYADSVKGRFTISRDNAKNSLYLQMNS |
| | | LRAEDTAVYYCAREGPNWGTHAFDIWGQGTMVTVSS |
| 1015 | ABL-CD3-101 | EVQLVESGGGLVNPGGSLRLSCAASGFTFSRYSMNWVRQAPG |
| | | KGLEWVSSISSSSSYIYYADSVKGRFTISRDNAKNSLYLQMNSL |
| | | RAEDTAVYYCANYRYNWNDYWGQGTLVTVSS |
| 1016 | ABL-CD3-102 | EVQLVESGGGLVKPGGSLRLSCAASGITFSSYSMNWVRQAPGK |
| | | GLEWVSSISSSSSYIYYADSVKGRFTISRDNAKNSLHLQMHSLR |
| | | AEDTAVYYCARERRELSFDYWGQGTLVTVSS |
| 1017 | ABL-CD3-103 | EVQLVESGGGLVKPGGSLRLSCAASGFTFSRYSMNWVRQAPG |
| | | KGLEWVSSISSSSSYISYADSVKGRFTISRDNAKNSLFLQMNSL |
| | | RAEDTAVYYCARVSYNSFDYWGQGTLVTVSS |
| 1018 | ABL-CD3-104 | EVQLVESGGGLVKPGGSLRLSCAASGFTFSRYSMNWVRQAPG |
| | | KGLEWVSSISSSSSYIYYADSVKGRFTISRDNAKNSLYLQMNSL |
| | | RAEDTAVYYCARAVYNYFDQWGQGTLVTVSS |
| 1019 | ABL-CD3-105 | EVQLVESGGGLVNPGGSLRLSCAASGFTFSRYSMNWVRQAPG |
| | | KGLEWVSSISSSSSYIYYADSVKGRFTISRDNAKNSLYLQMNSL |
| | | RAEDTAVYYCANYRYNWNDYWGQGTLVTVSS |
| 1020 | ABL-CD3-106 | EVQLVESGGGLVKPGGSLRLSCAASGFTFSSYSMNWVRQAPG |
| | | KGLEWVSSISRSSSYIFYADSVKGRFTISRDNAKNSLYLQMNSL |
| | | RAEDTAVYYCARERRNGFDYWGQGTLVTVSS |
| 1021 | ABL-CD3-107 | EVQLVQSGAEVKKPGESLRISCKGSGYSFTTYWIAWVRQMPG |
| | | KGLEWMGFIYPGDSDTIYSPSFQGQVTISADKSISTAYLQWSSL |
| | | RASDTAMYYCARQGYLDAFDIWGQGTMVTVSS |
| 1022 | ABL-CD3-108 | EVQLVESGGGLVKPGGSLRLSCAASGFTFSRYSMNWVRQAPG |
| | | KGLEWVSSISSSSRYIYYADSVKGRFTISRDNANNSLYLHMNSL |
| | | RAEDTAVYYCARALYNYFDYWGQGTLVTVSS |
| 1023 | ABL-CD3-109 | EVQLVESGGGLVKPGGSLRLSCAASGFTFSSYSMNWVRQAPG |
| | | KGLEWVSSISSSSSYIYYADSVKGRFTISRDNAKNSLYLQMNSL |
| | | RAEDTAVYYCANYRYNWNDYWGQGTLVTVSS |
| 1024 | ABL-CD3-110 | EVQLVESGGGLVKPGGSLRLSCAASGFTFSSYSMNWVRQAPG |
| | | KGLEWVSSISSSSSYIYYADSVKGRFTISRDNAKNSLYLQMNSL |
| | | RAEDTAVYYCARERRDLAFDIWGQGTMVTVSS |
| 1025 | ABL-CD3-111 | EVQLVESGGGLVKPGGSLRLSCAASGFTFSSYSMNWVRQAPG |
| | | KGLEWVSYISRSSTYIYYADSMKGRFTISRDNAKNSLYLQMNS |
| | | LRAEDTAVYYCAREGPNWGTHAFDIWGQGTMVTVSS |
| 1026 | ABL-CD3-112 | EVQLVESGGGLVKPGGSLRLSCAASGITFSSYSMNWVRQAPGK |
| | | GLEWVSSISSSSSYIYYADSVKGRFTISRDNAKNSLHLQMNSLR |
| | | AEDTAVYYCARERRELSFDYWGQGTLVTVSS |
| 1027 | ABL-CD3-113 | EVQLVESGGGLVKPGGSLRLSCAASGITFSSYSMNWVRQAPGK |
| | | GLEWVSSISSSSSYIYYADSVKGRFTISRDNAKNSLHLQMHSLR |
| | | AEDTAVYYCARERRELSFDYWGQGTLVTVSS |
| 1028 | ABL-CD3-1A | EVQLVESGGGLVKPGGSLRLSCAASGFTLSSYSMNWVRQAPG |
| | | KCLEWVSSISRSGSYIYYADSVKGRFTISRDNAKNSLYLQMNSL |
| | | RAEDTAVYYCAREKWELLYFDYWGQGTLVTVSS |
| 1029 | ABL-CD3-2A | EVQLVESGGGLVKPGGSLRLSCAASGFTFSSYSMNWVRQAPG |
| | | KCLEWVSSISRSTSYIYYADSVKGRFTISRDNAKNSLYLQMNSL |
| | | RAEDTAVYYCARGSRDAFDIWGQGTMVTVSS |
| 1030 | ABL-CD3-3A | EVQLVESGGGLVKPGGSLRLSCAASGFTFSSYSMNWVRQAPG |
| | | KCLEWVSYISRSSNYIHYADSVKGRFTIFRDNAKNSLYLQMNS |
| | | LRVEDTAVYYCARAERGSYAVDIWGQGTMVTVSS |
| 1031 | ABL-CD3-4A | VQLVESGGGLVKPGGSLRLSCAASGFTFSSYNMNWVRQAPGK |
| | | CLEWVSSISSSSSYIYYADSVKGRFTISRDNAKNSLYLQMNSLR |
| | | AEDTAVFYCARAERGSYAFDIWGQGTMVTVSS |
| 1032 | ABL-CD3-5A | EVQLVESGGGLVQPGGSLRLSCAASGFTFSRYSMNWVRQAPG |
| | | KCLEWVSYISSSSSSIYYADSVKGRFTISRDNAKNSLYLQMNSL |
| | | RDEDTAVYYCARDRRATLDYWGQGTLVTVSS |
| 1033 | ABL-CD3-6A | EVQLVDSGGGLVKPGGSLRLSCAASGFTFSSYSMNWVRQAPG |
| | | KCLEWVSSISSRGSYRNYADSVKGRFTISRDNAKNSLYLQMNS |
| | | LRAEDTAVYYCARDRRELLLDCWGQGTLVTVSS |
| 1034 | ABL-CD3-7A | EVQLVESGGGLVKPGGSLRLSCAASGFTFSRYSMNWVRQAPG |
| | | KCLKWVSSISSSGSYKYYADSVKGRFTISRDNAKNSLYLQMNS |
| | | LRAEDTAVYYCAREPPTLGMDYWGQGTLVTVSS |
| 1035 | ABL-CD3-8A | EVQLVESGGGLVKPGGSLRLSCAASGFTFSSYSMNWVRQAPG |
| | | KCLEWVSSISSSSSYIYYADSVKGRFTISRANAKNSLYLQMSSL |
| | | RAEDTAVYYCARDRRELLLDYWGQGTLVTVSS |
| 1036 | ABL-CD3-9A | EVQLVDSGGGLVKPGGSLRLSCAASGFTFSSYSMNWVRQAPG |
| | | KCLEWVSSVNSRGSYIYYADSVKGRFTISRDNAKNSLYLQMNS |
| | | LRADDTAVYYCARDRRELLLDYWGQGTLVTVSS |
| 1037 | ABL-CD3-10A | QVQLQQSGPGLVKPSQTLSLTCAISGDSVSCNSVAWNWIRQSP |
| | | SRCLEWLGRTYYRSKWHNEYAVSVKSRINIKPDTSKNQVSLQL |
| | | KSVTPEDTAVYYCARERKWELFLDYWGQGTLVTVSS |
| 1038 | ABL-CD3-11A | QVTLKESGPGILQPSQTLSLTCSFSGFSLSTFGMGVGWIRQPSG |
| | | KCLEWLAHIWWDDDKYYNPALKSRLTISKDTSKNQVFLKIAN |
| | | VDTADTATYYCARLGYYPYWFFDVWGTGTTVTVSS |
| 1039 | ABL-CD3-12A | EVQLVESGGGLVKPGGSLRLSCAASGFTFSSYTMNWVRQAPG |
| | | KCLEWVSSISRSSSYKYYADSVKGRFTISRDNAKNSLYLQMNS |
| | | LRAEDTAVYYCAREPPTLGMDYWGQGTLVIVSS |
| 1040 | ABL-CD3-13A | EVQLVESGGGLVQPGGSLRLSCAASGFTFSNYWMSWVRQAPG |
| | | KCLEWVANIKEDGSEKYYVDSVKGRFTISRDNAKNSLYLQMN |
| | | SLTAEDTSVYYCARERRTGYFDYWGQGTLVTVSS |
| 1041 | ABL-CD3-14A | EVQLVESGGGLVKPGGSLRLSCAASGFTCSSYSMTWVRQAPG |
| | | KCLEWVSSISRRSSYIYYADSVKGRFTISRDNAKNSLYLQLNSL |
| | | RAEDTAVYYCARERWELLYFDYWGQGTLVTVSS |
| 1042 | ABL-CD3-15A | EVQLVESGGGLVKPGGSLRLSCAASGFNCSSYSMNWVRQAPG |
| | | KCLEWVSSISRSSNYIYYADSVKGRFSISRDDAKNSLYLQMNSL |
| | | RAEDTAVYYCARERWIQLYSDYWGQGILVTVSS |
| 1043 | ABL-CD3-16A | EVQLVESGGGLVQPGGSLRLSCAASGFTFSRYWMTWVRQAPG |
| | | KCLEWVANIKEDGSEKYCVDSVKGRFTISRDNAKNSLYLQLNS |
| | | LRAEDTAVYYCARERRTGYFDYWGQGTLVTVSS |
| 1044 | ABL-CD3-17A | EVQLVESGGGLVQPGGSLRLSCAASGFTFSRYSINWVRQAPGK |
| | | CLEWVSYISSSSSTIYYADSVKGRFTISRDNAKNSLYLQMNSLR |
| | | DEDTAVYYCARERWELLYFDYWGQGTLVTVSS |
| 1045 | ABL-CD3-18A | EVQLVESGEGLVQPGGSLRLSCAASGFTFSSYAMHWVRQAPG |
| | | KCLEYVSAISSNGGRTYYADSVKGRFTISRDNSKNTLYLQMGS |
| | | LRAEDMAVYYCARGGSLDYWGQGTLVTVSS |
| 1046 | ABL-CD3-19A | EVQLVESGGGLVKPGGSLRLSCAASGFTLSSYSMNWVRQAPG |
| | | KCLEWVSSISRSSSYIYYADSVKGRFTISRDNAKNSLYLQMNSL |
| | | RAEDTAVYYCARERWELLYFDYWGQGTLVTVSS |
| 1047 | ABL-CD3-20A | EVQLVESGGGLVKPGGSLRLSCAASGLTFSTYSMNWVRQAPG |
| | | KCLEWVSSISSSGSYVYYADSVKGRFTMSRDNAKNSLYLQMN |
| | | SLRAEDTAVYYCARERRELLFDYWGQGTLVTVSS |
| 1048 | ABL-CD3-21A | EVQLVESGGGLVKPGGSLRLSCAASGFTFSSYSMNWVRQAPG |
| | | KCLEWVSSISRSSSYIYYADSVKGRFTLSRDNAKNSLYLQMNS |
| | | LRGEDTAIYYCGRDGKGAIFLDYWGQGTLVTVSS |
| 1049 | ABL-CD3-22A | EVQLVESGGGLVQPGGSLRLSCAASGFTFSSYSMNWVRQAPG |
| | | KCLEWVSYISSSSSTIYYADSVKGRFTISRDNAKNSLYLQMNSL |
| | | RDEDTAVYYCARDGPVDYGMDVWGQGTTVTVSS |
| 1050 | ABL-CD3-23A | EVHLVESGGGLVKPGGSLRLSCAASGFTFSSYSMNWVRQAPG |
| | | KCLEWVSSISSSSTYIYYADSVKGRFTISRDNAKNSLYLQMNSL |
| | | RAEDTAVYYCAREPRTLGMDYWGQGILVTVSS |
| 1051 | ABL-CD3-24A | EVQLVESGGGLVKPGGSLRLSCVASGFTFSSYTMNWVRQAPG |
| | | KCLEWVSSISRSGSYKYNADSVKGRFTISRDNAKNSLYLQMNS |
| | | LRVEDTAVYYCAREPPTLGMDYWGQGTLVTVSS |
| 1052 | ABL-CD3-25A | EVHLVESGGGLVKPGGSLRLSCAASGFTFSSYSMNWVRQAPG |
| | | KCLEWVSSISSSSTYIYYADSVKGRFTISRDNAKNSLYLQMNSL |
| | | RAEDTAVYYCAREPRTLGMDYWGQGILVTVSS |
| 1053 | ABL-CD3-26A | EVHLVESGGGLVKPGGSLRLSCAASGFTFSSYSMNWVRQAPG |
| | | KCLEWVSSISSSSTYIYYADSVKGRFTISRDNAKNSLYLQMNSL |
| | | RAEDTAVYYCAREPRTLGMDYWGQGILVTVSS |
| 1054 | ABL-CD3-27A | QVQLVQSGAEVKKPGASLKVSCKASGYTFTSYYMHWVRQAP |
| | | GQCLEWMGIINPSNGNTISAQKFQGRVTMTRDTSTSTVYMEMS |
| | | SLRSEDTAVYYCVRSWGYYVMDVWGQGTTVTVSS |
| 1055 | ABL-CD3-28A | QVQLQQSGPGLVKPSQTLSLTCAISGDSVSSNSVAWNWIRQSP |
| | | SRCLEWLGRTYYRSKWYNECAGSVKSRITINPDTSKNQFSLQL |
| | | NSVTPEDTAIYYCARERKWELFLDYWGQGTLVTVSS |
| 1056 | ABL-CD3-29A | EVQLVQSGAEVKKPGESLKISCKGSGYSFISYWIAWVRQMPGK |
| | | CLEWMGIIYPGDSETRYRPSFQGQVTISADKSINTAYLQWSSLK |
| | | AADTAMYYCARQGAYDGFDIWGQGTMVTVSS |
| 1057 | ABL-CD3-30A | QVQLQESGPGLVKPSETLSLTCTVSGGSINSYYWSWIRQSPGKC |
| | | LEWIGHIYYSGRTKYNPSLKSRVTISVDTSKNQFSLKLSSVTAA |
| | | DTAVYYSARENRNGAFDIWGQGTVVTVSS |
| 1058 | ABL-CD3-31A | EVQLVQSGAEVKKPGESLKISCKGSGYSFTNYWIGWVRQMPG |
| | | KCLEWMGIIYPADSDTTYSPSFQGQVTISADKSITTAYLQWSSL |
| | | KASDTAMYYCARHGGSYILDAFDIWGQGTMVTVSS |
| 1059 | ABL-CD3-32A | EVQLVESGGGLVKPGGSLRLSCEASGFTFSSYNMNWVRQAPG |
| | | KCLEWVSSISSSSSYIYYTDSVKGRFTISRDNAKNSLYLQMNSL |
| | | RAEDTAVYYCARAERGSYAFDFWGQGTMVTVSS |
| 1060 | ABL-CD3-33A | EVQLVESGGGLVKPGGSLRLSCAASGFTFSSYSMNWVRQAPG |
| | | KCLEWVSSISRSSSYIYYADSVKGRFTISRDNAKNSLYLQMNSL |
| | | RAEDTAVYYCAREPPTLGMDYWGQGTLVTVSS |
| 1061 | ABL-CD3-34A | EVHLVESGGGLVKPGGSLRLSCAASGFTFSRYSMNWVRQAPG |
| | | KCLEWVSSISRSSSYIYYADSVKGRFTISRDNAKNSLYLQMNSL |
| | | RAEDTAVYYCAREPRTLGMDYWGQGILVTVSS |
| 1062 | ABL-CD3-35A | EVQLVESGGGLVKPGGSLRLSCAASGFTFSSYSMNWVRQAPG |
| | | KCLEWVSSVNSRGSYIYYADSVKGRFTISRDNAKDSLYLQMNS |
| | | LRADDTAVYYCARDRRELLLDYWGQGTLVTVSS |
| 1063 | ABL-CD3-36A | EVQLVESGGGLVQRGGSLRLSCAASGFTFSNYWMSWVRQAPG |
| | | KCLEWVANIKEDGSEKYYVDSVKGRFTISRDNAKHSLYLQMN |
| | | SLRAEDTAVYYCARERRTGYFDYWGQGTLVTVSS |
| 1064 | ABL-CD3-37A | EVQLVESGGGLVQPGGSLRLSCAASGFSFSSYSMNWVRQAPG |
| | | KCLEWVSHIRSSTYSIYYADSVMGRFTISRDNAKNSLYLQMNS |
| | | LRDEDTAVYFCARAKTGGDAFDIWGQGTMVTVSS |
| 1065 | ABL-CD3-38A | QGQLQQWGAGLLKPSETLSLTCAVYGGSFSGYYWSWIRLPPG |
| | | KCLEWIGEINHSGNTNYNPSLKSRVTISVDTSKNQFSLQLSSVT |
| | | AADTAVYYCAKEVEIWGYFDYWGQGTLVTVSS |
| 1066 | ABL-CD3-39A | EVQLLESGGGLVQPGGSLRLSCAASGFTFSYYAMSWVRQAPG |
| | | KCLEWVSAISGSGGSTYYADSVKGRFTISRDNSKNTLYLQMNS |
| | | LRAEDTAVYYCAKEGIVGARAEYFQHWGQGTLVTVSS |
| 1067 | ABL-CD3-40A | EVQLLESGGGLVQPGGSLRLSCAASGFTFSYYAMSWVRQAPG |
| | | KCLEWVSAISGSGGSTYYADSVKGRFTISRDNSKNTLYLQMNS |
| | | LRAEDTAVYYCAKEGIVGARAEYFQHWGQGTLVTVSS |
| 1068 | ABL-CD3-41A | EVQLVESGGGLVKPGGSLRLSCAASGFTFSSYSMNWVRQAPG |
| | | KCLEWVSSVNSRGSYIYYADSVKGRFTISRDNAKDSLYLQMNS |
| | | LRADDTAVYYCARDRRELLLDYWGQGTLVTVSS |
| 1069 | ABL-CD3-42A | EVQLVESGGGLVQPGGSLRLSCAASGFTFKSYSINWVRQAPGK |
| | | CLEWVSYISSSSSTIYYADSVKGRFTISRDNAKNSLYLQMNSLR |
| | | DEDTAVYYCARDGPVDYGMDVWGQGTTVTVSS |
| 1070 | ABL-CD3-43A | EVQLVQSGAEVKKPGESLKISCKGSEYSFTTYWIAWVRQMPG |
| | | KCLEWMGIIYPGDSDTTYSPSFQGQVTISADKSISIAFLQWSSLK |
| | | ASDTAMYYCARQGELDAFDIWGQGTMVTVSS |
| 1071 | ABL-CD3-44A | EVQLVESGGGLVQPGGSLRLSCAASGFTFSTYWMSWVRQAPG |
| | | KCLEWVANIKQDGSEKYYVDSVKGRFTISRDNAKNSLYLQMN |
| | | SLRAEDTAVYYCATGSFDYWGQGTLVTVSS |
| 1072 | ABL-CD3-45A | EVQLVESGGGLVQPGGSLRLSCAASGFTFSSYWMSWVRQAPG |
| | | KCLEWVANIKEDGSEKYYVDSVKGRFTISRDNAKNSLYLQMN |
| | | SLRAEDTAVYYCARERRTGYLDYWGQGTLVTVSS |
| 1073 | ABL-CD3-46A | EVQLVESGGGLVKPGGSLRLSCSASGFTFSTYTMNWVRQAPG |
| | | KCLDWVSSISKSSTYIYYADSVKGRFTISRDNAKNSLYLQMNS |
| | | LRAEDTAVYYCARERRTLAFDIWGQGTMVTVSS |
| 1074 | ABL-CD3-47A | EVQLVESGGGLVQPGGSLRLSCAASGFTFSRYSMNWVRQAPG |
| | | KCLEWVSYISRSSTTIYYADSVKGRFTISRDNAKNSLYLQMNSL |
| | | RDEDAAVYYCARERWELLYFDYWGQGTLVTVSS |
| 1075 | ABL-CD3-48A | EVQLVESGGGLVKPGGSLRLSCAASGFTFSSYTMNWVRQAPG |
| | | KCLEWVSSISGSGSYIYYADSVKGRFTISRDNAKNSLYLQMNS |
| | | LRAEDTAVYYCAREPPTLGMDYWGQGTLVTVSS |
| 1076 | ABL-CD3-49A | EVQLVESGGGLVKPGGSLRLSCSASGFTFSTYTMNWVRQAPG |
| | | KCLEWVSSISKSSSYIYYTDSVKGRFTISRDNAKNSLYLQMNSL |
| | | RAEDTAVYYCARERRTLAFDIWGQGTMVTVSS |
| 1077 | ABL-CD3-50A | QVQLQQSGPGLVKPSQTLSLTCAISGDSVSSNSVAWNWIRQSP |
| | | SRCLEWLGRTYSRSKWYNDYAVSVKSRITINPDTSKNQFSLEL |
| | | NSVTPEDTAVYYCARERKWELFLDYWGQGTLVTVSS |
| 1078 | ABL-CD3-51A | EVQLVESGGGLVKPGGSLRLSCAASGFTFSSYSMNWVRQAPG |
| | | KCLEWVSSISRSSSYIYYADSVKGRFTISRDNAKNSLYLQMNSL |
| | | RAEDTAVYYCARERWELLYFDYWGQGTLVTVSS |
| 1079 | ABL-CD3-52A | EVQLVESGGGLVKPGGSLRLSCAASGFTFSYYSMNWVRQAPG |
| | | KCLEWVSSISRSTTYIYYADSVKGRFTISRDNAKNSLYLQMNSL |
| | | RAEDTAVYYCARDGKGAIFLDYWGQGTLVTVSS |
| 1080 | ABL-CD3-53A | EVQLVESGGGLVKPGGSLRLSCAASGFTFSSYTMNWVRQAPG |
| | | KCLEWVSSITTSSSYIYYADSVKGRFTISRDNAKNSLYLQMNSL |
| | | RAEDTAVYYCARVTFDAFDIWGQGTMVTVSS |
| 1081 | ABL-CD3-54A | EVQLVESGGGLVKPGGSLRLSCAASGFTFSSYSMNWVRQAPG |
| | | KCLEWVSSISRSSRYIFYADSVRGRFTISRDNAKNSLYLQMNSL |
| | | RAEDTAVYYCAREKRELLFDYWGQGTLVTVSS |
| 1082 | ABL-CD3-55A | EVQLVESGGGLVKPGGSLRLSCAASGFTFSIYSMNWVRQAPGK |
| | | CLEWVSSISSGSSYIYYADSVKGRFTISRDNAKSSLFLQMNSLR |
| | | AEDTAVYYCAREKRELLFDYWGQGTLVTVSS |
| 1083 | ABL-CD3-56A | EVQLVESGGGLVKPGGSLRLSCVASGFTFSRYSMNWVRQAPG |
| | | KCLEWVSSISSSSRYTYYADSLKGRFIMSRDNAKNSLFLQMNS |
| | | LRAEDTAVYYCARNLYNALDYWGQGTLVTVSS |
| 1084 | ABL-CD3-57A | EVQLVESGGGLVKPGGSLRLSCAASGFTFSSYTMNWVRQAPG |
| | | KCLEWVSSISTSSRYIYYTDSVKGRFTISRDDAKNSLYLQMNSL |
| | | RAEDTAVYYCTRNWGYDVFDIWGQGTMVIVSS |
| 1085 | ABL-CD3-58A | EVQLVESGGGLVKPGGSLRLSCVVSGFTFSNYNMNWVRQAPG |
| | | KCLEWVSSSSRSSYIYYADSVKGRFTISRDNAKNSLYLQMNSL |
| | | RAEDTAIYYCAREKWELLFFDYWGQGTLVTVSS |
| 1086 | ABL-CD3-59A | EVQLVESGGGLVKPGGSLRLSCTASGFTLSSYSMNWVRQAPG |
| | | KCLEWVSSLSRSSNYIYYAVSVKGRFTISRDNAKNSLYLQMNS |
| | | LRAEDTAVYYCARERWELLYFDYWGQGTLVTVSS |
| 1087 | ABL-CD3-60A | EVQLVESGGGLVKPGGSLRLSCAASGFTFSIYSMNWVRQAPGK |
| | | CLEWVSSISSGSSYIYYADSVKGRFTISRDNAKSSLFLQMNSLR |
| | | AEDTAVYYCAREKRELLFDYWGQGTLVTVSS |
| 1088 | ABL-CD3-61A | EVQLVESGGGLVKPGGSLRLSCAASGFTFSSYSMNWVRQAPG |
| | | KCLEWVSSISRSGTYIYYADSVKGRFTISRDNAKNSLYLQMNS |
| | | LRAEDTAVYYCARERWELLYFDYWGQGTLVTVSS |
| 1089 | ABL-CD3-62A | EVQLVESGGGLVKPGGSLRLSCAASGFTFSSYSMNWVRQAPG |
| | | KCLEWVSSISRSSGYIFYADSVKGRFTISRDNAKNSLYLQMNSL |
| | | RAEDTAVYYCVRERVGAGFDYWGQGTLVTVSS |
| 1090 | ABL-CD3-63A | QVQLQESGPGLVKPSETLSLTCTVSGGSISSYYWSWIRQPAGKC |
| | | LEWIGRIYTSGSTKYNPSLKSRVTMSVDTSKNQFSLKLISVTAA |
| | | DTAVYFCARERIRDLYFDYWGQGTLVTVSS |
| 1091 | ABL-CD3-64A | EVQVAESGGGLVKPGGSLRLSCAASGFTFSRYSMNWVRQAPG |
| | | KCLEWVSSISSSGSYTYYADSVKGRFTISRDNAKNSLYLQMNS |
| | | LRAEDTAVYYCVRERGKGAGMDVWGQGTTVTVSS |
| 1092 | ABL-CD3-65A | QVQLQQWGAGLLKPSETLSLTCAVYGGSFSGYYWSWIRQPPG |
| | | KCLEWIGEIQHSGSTNYNPSLKSRVTISVDTSKNQFSLKLSSVT |
| | | AADTAVYYCAREYYYYGMDVWGQGTTVTVSS |
| 1093 | ABL-CD3-66A | QVQLQESGPGLVKPSETLSLTCTVSGGSISSYYWSWIRQPAGKC |
| | | LEWIGRIYASGSTYYNPSLKSRVTMSVDTSKNQFSLKLSSVTAA |
| | | DTAVYYCAREKRSRLYFDYWGQGTLVTVSS |
| 1094 | ABL-CD3-67A | EVQLVESGGGLVKPGGSLRLSCAASGFTVSSYSMNWVRQAPG |
| | | KCLEWVSSISRSSSYIYYADSVKGRFTISRDNAKNSLFLQMNSL |
| | | RAEDTAVYYCARERWELLYFDYWGQGTLVTVSS |
| 1095 | ABL-CD3-68A | EVQLVESGGGLVKPGGSLRLSCAASGFTFSSSIMNWVRQAPGK |
| | | CLEWVSSISGSSSYIYYADSVKGRFTISRDNAKNSQYLQMKSLR |
| | | AEDTAVYYCARERWNLLYFDYWGQGTLVTVSS |
| 1096 | ABL-CD3-69A | EVQLVESGGGLVKPGGSLRLSCAASGFTFSTYTMNWVRQAPG |
| | | KCLEWVSTITSSSRYIYYADSVKGRFTISRDNAKNSLYLQMNSL |
| | | RAEDTAVYYCARVTFDAFDIWGQGTMVTVSS |
| 1097 | ABL-CD3-70A | EVQLVESGGGLVKPGGSLRLSCAASGFTFSTYIMNWVRQAPG |
| | | KCLEWVSSITSSSSYIYYADSVKGRFTISRDNAKNSLYLQMNSL |
| | | RAEDTAVYYCAREKWELLFFDYWGQGTLVTVSS |
| 1098 | ABL-CD3-71A | QVQLQESGPGLVKPSETLSLTCTVSGGSINSYYWSWIRQPAGK |
| | | CLEWIGRIYTSGSTNYNPSLKSRVTMSVDTSKKQFSLKLSSVTA |
| | | ADTAVYYCARERISRLYFDYWGQGILVTVSS |
| 1099 | ABL-CD3-72A | EVQLVESGGGLVKPGGSLRLSCAASGFTFSTYIMNWVRQAPG |
| | | KCLEWVSSISSSRSYIYYADSVKGRFTISRDNAKNSLYLQMNSL |
| | | RAEDTAVYYCAREDRYRLFFDYWGQGTLVTVSS |
| 1100 | ABL-CD3-73A | EVQLVESGGGLVKPGGSLRLSCAASGFTITSYSMNWVRQAPG |
| | | KCLEWVSTFSRSSTYIYYADSVKGRFTISRDNAKNSLYLQMNS |
| | | LRAEDTAVYYCAREKWDLLYFDYWGQGTLVTVSS |
| 1101 | ABL-CD3-74A | EVQLVESGGGLVKPGGSLRLSCAASGFTFSSYSMKWVRQAPG |
| | | KCLEWVSSISSSSSYIYYADSVKGRFTISRDNAKNSLYLQMNSL |
| | | RAEDTAVYYCARDRGELTLWYWGQGTLVTVSS |
| 1102 | ABL-CD3-75A | EVQLVESGGGLVKPGGSLRLSCAASGFTFSTYSLNWVRQAPGK |
| | | CLEWVSSISVSSRYIYFADSVKGRFTISRDNAKNSLYLQMNSLR |
| | | AEDTAVYYCARDRRELLLDYWGQGTLVTVSS |
| 1103 | ABL-CD3-76A | EVQLVESGGGLVKPGGSLRLSCAASGFTFSNYYMNWVRQAPG |
| | | KCLERVSSISSSSNYIYYADSVKGRFTISRDNAKNSLYLQMNSL |
| | | RAEDTAVYYCARDRRELPLDYWGQGTVVTVSS |
| 1104 | ABL-CD3-77A | EVQLVESGGGLVKPGGSLRLSCAASGFTFSSYSMNWVRQAPG |
| | | KCLEWVSSISSSSSYIYYADSVKGRFTISRDNAKNSLYLQMNSL |
| | | RAEDTAVYYCARDGELGIGTYFDYWGQGTLVTVSS |
| 1105 | ABL-CD3-78A | EVQLVESGGGLVKPGGSLRLSCAASGFTVSNYSMNWVRQAPG |
| | | KCLEWVSCISRSSSYIYYADSVKGRFTISRDNAKNSLFLQMNSL |
| | | RAEDTAVYYCARERWELLYFDYWGQGTLVTVSS |
| 1106 | ABL-CD3-79A | EVQLVESGGGLVKPGGSLRLSCAASGFTVSNYSMNWVRQAPG |
| | | KCLEWVSSISRSSSYIYYADSVKGRFTISRDNAKNSLFLQMNSL |
| | | RAEDTAVYYCARERWELLYFDYWGQGTLVTVSS |
| 1107 | ABL-CD3-80A | EVQLVESGGGLVKPGGSLRLSCAASGFTFSGYSMNWVRQAPG |
| | | KCLEWVSSISSRSRYIYYADSVQGRFTISRDNSKNSLFLQMNSL |
| | | RAEDTAVYYCGRDWRELLLDYWGQGTLVTVSS |
| 1108 | ABL-CD3-81A | QVQLQESGPGLVKPSQTLSLTCTVSGGSISSGGYYWSWIRQHP |
| | | GKCLEWIGYIYYSGSTYYNPSLKSRVTISVDTSKDQFSLKLSSV |
| | | TAADTAVYYCARERKTSLPFDIWGQGTMVTVSS |
| 1109 | ABL-CD3-82A | EVQLVESGGGLVKPGGSLRLSCAASGFTFSSYSMNWVRQAPG |
| | | KCLEWVSSISSSSSYIYYADSVKGRFTISRDNAKNSLYLQMNSL |
| | | RAEDTAVYYCARDRRELLLDYWGQGTLVTVSS |
| 1110 | ABL-CD3-83A | EVQLVESGGGLVKPGGSLRLSCAASGFTFSSYSMNWVRQAPG |
| | | KCLEWVSTISRSGGYIYDADSVKGRFTISRDNAKNSLYLQMNS |
| | | LRAEDTAVYYCARDFNWTFDNWGQGTLVTVSS |
| 1111 | ABL-CD3-84A | EVQLVESGGGLVKPGGSLRLSCAASGFTFSNFIMNWVRQAPGK |
| | | CLEWVSSISGSSNYIYYADSVKGRFTISGDNAKNSLYLQMNSL |
| | | RAEDTAVYYCAREKWVLLFFDYWGQGTLVTVSS |
| 1112 | ABL-CD3-85A | EVQLVESGGGLVKPGGSLRLSCAASGFTFSSYSMNWVRQAPG |
| | | KCLEWVSSISRRSGYIYYADSVKGRFTLSRDNAKKSLYLQMNS |
| | | LRAEDTAVYYCARDLNWAFDYWGQGTLVTVSS |
| 1113 | ABL-CD3-85B | EVQLVESGGGLVKPGGSLRLSCAASGFTFSSYSMNWVRQAPG |
| | | KCLEWVSSISRRSGYIYYADSVKGRFTLSRDNAKKSLYLQMNS |
| | | LRAEDTAVYYCARDLNWAFDYWGQGTLVTVSS |
| 1114 | ABL-CD3-86A | EVQLVESGGGLVKPGGSLRLSCAASGFTFSRYSMNWVRQAPG |
| | | KCLEWVSTISRSSRYIYYGDSVKGRFTISRDNAKNSLLLQMNSL |
| | | RAEDTAVYYCARDHNWSFDYWGKGILVTVSS |
| 1115 | ABL-CD3-87A | EVQLVESGGGLVKFGGSLRLSCAASGFTFSRYSMNWVRQAPG |
| | | KCLEWVSSISSSSSYKYYADSVKGRFTISRDNAKNSLYLQMNS |
| | | LRAEDTAVYYCAREGGNSHGMDVWGQGTTVTVSS |
| 1116 | ABL-CD3-88A | EVQLVESGGGLVQPGGSLRLSCAASGFTFSRYWMSWVRQAPG |
| | | KCLEWVANIKEDGSEKYYVDSVKGRFTISRDNAKNSLYLQMN |
| | | SLRAEDTAVYFCARERRTGYFDYWGQGTLVTVSS |
| 1117 | ABL-CD3-89A | EVQLVESGGGLVQPGGSLRLSCAASGFTFSSYWMSWVRQAPG |
| | | KCLEWVSSISSRRGYKYHADSVKGRFTISRDNAKNSLYLQMNS |
| | | LRAEDTAVYYCARVPYSGSYFDYWGQGTLVTVSS |
| 1118 | ABL-CD3-90A | EVQLVESGGGLVKPGGSLRLSCAASGFTFSSYSMNWVRQAPG |
| | | KCLEWVSSISSSSSYIYYADSVKGRFTISRDNAKNSLYLQMNSL |
| | | RAEDTAVYYCTNYRYNWNDYWGQGTLVTVSS |
| 1119 | ABL-CD3-91A | EVQLVESGGGLVKPGGSLRLSCAASGFTFSYYSMNWVRQAPG |
| | | KCLEWVSSISSSSTYIYYADSVKGRFTISRDNAKNSLYLQMNSL |
| | | RAEDTAVYYCANYRYNWNDYWGQGTLVTVSS |
| 1120 | ABL-CD3-92A | EVQLVESGGGLVKPGGSLRLSCAASGFTFSSYSMNWVRQAPG |
| | | KCLEWVSYISSSSRYIYYADSVKGRFTISRDNAKNSLYLQMNSL |
| | | RAEDTAVYYCAREGPNWGTHAFDIWGQGTMVTVSS |
| 1121 | ABL-CD3-93A | EVQLVESGGALVKPGGSLRLSCAASGFTFSRYSMNWVRQAPG |
| | | KCLEWVSSISSSSSYIYYADSVKGRFTISRDNAKNSLYLQMNSL |
| | | RAEDTAVYYCANYRYNWNDYWGQGTLVAVSS |
| 1122 | ABL-CD3-94A | EVQLVESGGGLVKPGGSLRLSCSASGFTFSSYSMNWVRQAPG |
| | | KCLEWVSSISRSSSYIFYADSVKGRFTISRDNAKNSLYLQMNSL |
| | | RAEDTAVYYCARERRNGFDYWGQGTLVTVSS |
| 1123 | ABL-CD3-95A | EVQLVESGGGLVKPGGSLRLSCAASGFTFSRYSMNWVRQAPG |
| | | KCLEWVSSISRSSSYIYYADSVKGRFTISRDNAKNSLYLQMNSL |
| | | RAEDTAVYYCANYRYNWNDYWGQGTLVTVSS |
| 1124 | ABL-CD3-96A | EVQLVESGGGLVKPGGSLRLSCAASGFTFSRYSMNWVRQAPG |
| | | KCLEWVSSISSSSSYIYYADSVKGRFTISRDNAKNSLYLQMNSL |
| | | RAEDTAVYYCARAVYNYFDQWGQGTLVTVSS |
| 1125 | ABL-CD3-97A | EVQLVESGGGLVKPGGSLRLSCAASGFTFSSYSMNWVRQAPG |
| | | KCLEWVSYISRSSTYIYYADSMKGRFTISRDNAKNSLYLQMNS |
| | | LRAEDTAVYYCAREGPNWGTHAFDIWGQGTMVTVSS |
| 1126 | ABL-CD3-98A | EVQLVESGGGLVKPGGSLRLSCAASGLTFSSYSMNWVRQAPG |
| | | KCLEWVSSISSSSSYIYYADSVKGRFTISRDNAKNSLYLQMNSL |
| | | RAEDTAVYYCARERRELAFDIWGQGTMVTVSS |
| 1127 | ABL-CD3-99A | EVQLVESGGGLVKPGGSLRLSCAASGITFSSYSMNWVRQAPGK |
| | | CLEWVSSISSSSSYIYYADSVKGRFTISRDNAKNSLHLQMNSLR |
| | | AEDTAVYYCARERRELSFDYWGQGTLVTVSS |
| 1128 | ABL-CD3-100A | EVQLVESGRGLVNPGGSLRLSCAASGFTFSSYSMNWVRQAPG |
| | | KCLEWVSYISSSSNYIYYADSVKGRFTISRDNAKNSLYLQMNS |
| | | LRAEDTAVYYCAREGPNWGTHAFDIWGQGTMVTVSS |
| 1129 | ABL-CD3-101A | EVQLVESGGGLVNPGGSLRLSCAASGFTFSRYSMNWVRQAPG |
| | | KCLEWVSSISSSSSYIYYADSVKGRFTISRDNAKNSLYLQMNSL |
| | | RAEDTAVYYCANYRYNWNDYWGQGTLVTVSS |
| 1130 | ABL-CD3-102A | EVQLVESGGGLVKPGGSLRLSCAASGITFSSYSMNWVRQAPGK |
| | | CLEWVSSISSSSSYIYYADSVKGRFTISRDNAKNSLHLQMHSLR |
| | | AEDTAVYYCARERRELSFDYWGQGTLVTVSS |
| 1131 | ABL-CD3-103A | EVQLVESGGGLVKPGGSLRLSCAASGFTFSRYSMNWVRQAPG |
| | | KCLEWVSSISSSSSYISYADSVKGRFTISRDNAKNSLFLQMNSL |
| | | RAEDTAVYYCARVSYNSFDYWGQGTLVTVSS |
| 1132 | ABL-CD3-104A | EVQLVESGGGLVKPGGSLRLSCAASGFTFSRYSMNWVRQAPG |
| | | KCLEWVSSISSSSSYIYYADSVKGRFTISRDNAKNSLYLQMNSL |
| | | RAEDTAVYYCARAVYNYFDQWGQGTLVTVSS |
| 1133 | ABL-CD3-105A | EVQLVESGGGLVNPGGSLRLSCAASGFTFSRYSMNWVRQAPG |
| | | KCLEWVSSISSSSSYIYYADSVKGRFTISRDNAKNSLYLQMNSL |
| | | RAEDTAVYYCANYRYNWNDYWGQGTLVTVSS |
| 1134 | ABL-CD3-106A | EVQLVESGGGLVKPGGSLRLSCAASGFTFSSYSMNWVRQAPG |
| | | KCLEWVSSISRSSSYIFY ADSVKGRFTISRDNAKNSLYLQMNSL |
| | | RAEDTAVYYCARERRNGFDYWGQGTLVTVSS |
| 1135 | ABL-CD3-107A | EVQLVQSGAEVKKPGESLRISCKGSGYSFTTYWIAWVRQMPG |
| | | KCLEWMGFIYPGDSDTIYSPSFQGQVTISADKSISTAYLQWSSL |
| | | RASDTAMYYCARQGYLDAFDIWGQGTMVTVSS |
| 1136 | ABL-CD3-108A | EVQLVESGGGLVKPGGSLRLSCAASGFTFSRYSMNWVRQAPG |
| | | KCLEWVSSISSSSRYIYYADSVKGRFTISRDNANNSLYLHMNSL |
| | | RAEDTAVYYCARALYNYFDYWGQGTLVTVSS |
| 1137 | ABL-CD3-109A | EVQLVESGGGLVKPGGSLRLSCAASGFTFSSYSMNWVRQAPG |
| | | KCLEWVSSISSSSSYIYYADSVKGRFTISRDNAKNSLYLQMNSL |
| | | RAEDTAVYYCANYRYNWNDYWGQGTLVTVSS |
| 1138 | ABL-CD3-110A | EVQLVESGGGLVKPGGSLRLSCAASGFTFSSYSMNWVRQAPG |
| | | KCLEWVSSISSSSSYIYYADSVKGRFTISRDNAKNSLYLQMNSL |
| | | RAEDTAVYYCARERRDLAFDIWGQGTMVTVSS |
| 1139 | ABL-CD3-111A | EVQLVESGGGLVKPGGSLRLSCAASGFTFSSYSMNWVRQAPG |
| | | KCLEWVSYISRSSTYIYYADSMKGRFTISRDNAKNSLYLQMNS |
| | | LRAEDTAVYYCAREGPNWGTHAFDIWGQGTMVTVSS |
| 1140 | ABL-CD3-112A | EVQLVESGGGLVKPGGSLRLSCAASGITFSSYSMNWVRQAPGK |
| | | CLEWVSSISSSSSYIYYADSVKGRFTISRDNAKNSLHLQMNSLR |
| | | AEDTAVYYCARERRELSFDYWGQGTLVTVSS |
| 1141 | ABL-CD3-113A | EVQLVESGGGLVKPGGSLRLSCAASGITFSSYSMNWVRQAPGK |
| | | CLEWVSSISSSSSYIYYADSVKGRFTISRDNAKNSLHLQMHSLR |
| | | AEDTAVYYCARERRELSFDYWGQGTLVTVSS |

TABLE 1F
scFv Sequences

| | | |
|---|---|---|
| SEQ ID | | |
| NO: | Name | Sequence |

Full scFv sequence

| | | |
|---|---|---|
| 1155 | ABL-CD3- | EVQLVESGEGLVQPGGSLRLSCAASGFTFSSYAMHWVRQAPGKCL |
| | 18A_scFv_ | EYVSAISSNGGRTYYADSVKGRFTISRDNSKNTLYLQMGSLRAED |
| | VH-VL | MAVYYCARGGSLDYWGQGTLVTVSSGGGGSGGGGSGGGGSGGG |
| | | GSDIVMTQTPLSSPVTPGQPASISCRSSQSLVHSDGNTYLNWLQQR |
| | | PGQPPRLLIYKISDRFSGVPDRFSGSGAGTDFTLKIGRVEAEDVGVY |
| | | YCMQSTQFPITFGCGTRLEIK |
| 1156 | ABL-CD3- | EVQLVESGGGLVKPGGSLRLSCAASGLTFSTYSMNWVRQAPGKCL |
| | 20A_scFv_ | EWVSSISSSGSYVYYADSVKGRFTMSRDNAKNSLYLQMNSLRAED |
| | VH-VL | TAVYYCARERRELLFDYWGQGTLVTVSSGGGGSGGGGSGGGGSG |
| | | GGGSNFMLTQPHSVSESPGKTVTISCTRSSGSIASNYVQWYQQRPG |
| | | SSPTSVIYEDNQRPSGVPDRFSGSIDSSSNSASLTISGLKTEDEADYY |
| | | CQSYDRSNRVFGCGTKLTVL |
| 1157 | ABL-CD3- | EVQLVESGGGLVKPGGSLRLSCAASGFTFSSYSMNWVRQAPGKCL |
| | 21A_scFv_ | EWVSSISRSSSYIYYADSVKGRFTLSRDNAKNSLYLQMNSLRGEDT |
| | VH-VL | AIYYCGRDGKGAIFLDYWGQGTLVTVSSGGGGSGGGGSGGGGSG |
| | | GGGSSYELTQPPSVSVSPGQTARITCSGDALPKKYAYWYQQKSGQ |
| | | APVLVIYEDSKRPSGIPERFSGSSSGTMATLTISGAQVEDEADYYCY |
| | | STHSSGNHRVFGCGTKLTVL |
| 1158 | ABL-CD3- | EVQLVESGGGLVQPGGSLRLSCAASGFTFSSYSMNWVRQAPGKCL |
| | 22A_scFv_ | EWVSYISSSSSTIYYADSVKGRFTISRDNAKNSLYLQMNSLRDEDT |
| | VH-VL | AVYYCARDGPVDYGMDVWGQGTTVTVSSGGGGSGGGGSGGGGS |
| | | GGGGSSYELTQPPSVSVSPGQTARITCSGDALPKQYTYWYQQKPG |
| | | QAPVLVIYKDSERPSGIPERFSGSSSGTTVTLTISGVQAEDEADYYC |
| | | QSADSSGTYRVFGCGTKLTVL |
| 1159 | ABL-CD3- | QVQLVQSGAEVKKPGASLKVSCKASGYTFTSYYMHWVRQAPGQ |
| | 27A_scFv_ | CLEWMGIINPSNGNTISAQKFQGRVTMTRDTSTSTVYMEMSSLRSE |
| | VH-VL | DTAVYYCVRSWGYYVMDVWGQGTTVTVSSGGGGSGGGGSGGG |
| | | GSGGGGSQTVVTQEPSFSVSPGGTVTLTCGLNSGSVSTSYYPSWYQ |
| | | QTPGQTPRTLIYSTNTRSSGVPDRFSGSILGNKAALTITGAQADDES |
| | | DYYCVLYMGSGIWVFGCGTKLTVL |
| 1160 | ABL-CD3- | EVQLVQSGAEVKKPGESLKISCKGSGYSFISYWIAWVRQMPGKCL |
| | 29A_scFv_ | EWMGIIYPGDSETRYRPSFQGQVTISADKSINTAYLQWSSLKAADT |
| | VH-VL | AMYYCARQGAYDGFDIWGQGTMVTVSSGGGGSGGGGSGGGGSG |
| | | GGGSQSVLTQPPSASGTPGQRVTISCSGSSSNIGSNYVYWYQQLPG |
| | | TAPKLLIYSNNQRPSGVPDRFSGSKSGTSASLAISGLRSEDEADYYC |
| | | EAWDDSLSGPVFGCGTKLTVL |
| 1161 | ABL-CD3- | EVQLVQSGAEVKKPGESLKISCKGSGYSFTNYWIGWVRQMPGKCL |
| | 31A_scFv_ | EWMGIIYPADSDTTYSPSFQGQVTISADKSITTAYLQWSSLKASDT |
| | VH-VL | AMYYCARHGGSYILDAFDIWGQGTMVTVSSGGGGSGGGGSGGG |
| | | GSGGGGSDIVMTQSPLSLPVTPGEPASISCRSSQSLLHSNGYNYLD |
| | | WYLQKPGQSPQLLIYLGSNRASGVPDRFSGSGSGTDFTLKISRVEA |
| | | EDVGFYYCMQPLQTPYTFGCGTKLEIK |
| 1162 | ABL-CD3- | EVHLVESGGGLVKPGGSLRLSCAASGFTFSRYSMNWVRQAPGKCL |
| | 34A_scFv_ | EWVSSISRSSSYIYYADSVKGRFTISRDNAKNSLYLQMNSLRAEDT |
| | VH-VL | AVYYCAREPRTLGMDYWGQGILVTVSSGGGGSGGGGSGGGGSGG |
| | | GGSNFMLTQPHSVSESPGKTVTISCTRSSGSIASNYVQWYQQRPGS |
| | | SPTTVIYEDNQRPSGVPDRFSGSIDSSSNSASLTISGLKTEDEADYYC |
| | | QSYDSSNRVFGCGTKLTVL |
| 1163 | ABL-CD3- | EVQLVESGGGLVQRGGSLRLSCAASGFTFSNYWMSWVRQAPGKC |
| | 36A_scFv_ | LEWVANIKEDGSEKYYVDSVKGRFTISRDNAKHSLYLQMNSLRAE |
| | VH-VL | DTAVYYCARERRTGYFDYWGQGTLVTVSSGGGGSGGGGSGGGG |
| | | SGGGGSDIQMTQSPSTLSASVGDRVTITCRASQSISSWLAWFQQKT |
| | | GKAPKLLIYKASSLESGVPSRFSGSGSGTEFTLTISSLQPDDFATYYC |
| | | QQYISYSWTFGCGTKVEIK |
| 1164 | ABL-CD3- | EVQLVESGGGLVQPGGSLRLSCAASGFSFSSYSMNWVRQAPGKCL |
| | 37A_scFv_ | EWVSHIRSSTYSIYYADSVMGRFTISRDNAKNSLYLQMNSLRDEDT |
| | VH-VL | AVYFCARAKTGGDAFDIWGQGTMVTVSSGGGGSGGGGSGGGGS |
| | | GGGGSDIQMTQSPSTLSASVGDRVTITCRASQSISSWLAWYQQKPG |
| | | KAPKLLIHKASSLESGVPSRFSGSGSGTEFTLTISSLQPDDFATYYCQ |
| | | QYNSYWTFGCGTKVEIK |
| 1165 | ABL-CD3- | EVQLLESGGGLVQPGGSLRLSCAASGFTFSYYAMSWVRQAPGKCL |
| | 39A_scFv_ | EWVSAISGSGGSTYYADSVKGRFTISRDNSKNTLYLQMNSLRAED |
| | VH-VL | TAVYYCAKEGIVGARAEYFQHWGQGTLVTVSSGGGGSGGGGSGG |
| | | GGSGGGGSDVVMTQSPLSLPVTLGQPASISCRSSQSLVYSDGNTYL |
| | | TWFQQRPGQSPRRLIYKVSNRDSGVPDRFSGSGSGTDFTLKISRVE |
| | | AEDVEIYYCMQGTHWPLTFGCGTKVEIK |
| 1166 | ABL-CD3- | EVQLLESGGGLVQPGGSLRLSCAASGFTFSYYAMSWVRQAPGKCL |
| | 40A_scFv_ | EWVSAISGSGGSTYYADSVKGRFTISRDNSKNTLYLQMNSLRAED |
| | VH-VL | TAVYYCAKEGIVGARAEYFQHWGQGTLVTVSSGGGGSGGGGSGG |
| | | GGSGGGGSQSVLTQPPSASGTPGQRVTISCSGSSSNIGSNTVNWYQ |
| | | QFPGTAPKLLIYSNNQRPSGVPDRFSGSKSGTSASLAISGLQSEDEA |
| | | DYYCAPWDDSLNGVVFGCGTKLTVL |
| 1167 | ABL-CD3- | EVQLVESGGGLVKPGGSLRLSCAASGFTFSSYSMNWVRQAPGKCL |
| | 41A_scFv_ | EWVSSVNSRGSYIYYADSVKGRFTISRDNAKDSLYLQMNSLRADD |
| | VH-VL | TAVYYCARDRRELLLDYWGQGTLVTVSSGGGGSGGGGSGGGGSG |
| | | GGGSSYELTQPPSVSVSPGQTARITCSGDALPKKYAYWYQQKSGQ |
| | | APVLVIYEDSKRPSGIPERFSGSSSGTMATLTISGAQVEDEADYYCY |
| | | STDSSGNHSWVFGCGTKLTVL |
| 1168 | ABL-CD3- | EVQLVQSGAEVKKPGESLKISCKGSEYSFTTYWIAWVRQMPGKCL |
| | 43A_scFv_ | EWMGIIYPGDSDTTYSPSFQGQVTISADKSISIAFLQWSSLKASDTA |
| | VH-VL | MYYCARQGELDAFDIWGQGTMVTVSSGGGGSGGGGSGGGGSGG |
| | | GGSQSALTQPASVSGSPGQSITISCTGTSSDVGSYNLVSWYQQHPG |
| | | KAPKLMIYEDIRRPSGVSNRFSASKSGDTASLTISGLQAEDEADYY |
| | | CSSYAGSTTFAIFGCGTKLTVL |
| 1169 | ABL-CD3- | EVQLVESGGGLVQPGGSLRLSCAASGFTFSTYWMSWVRQAPGKC |
| | 44A_scFv_ | LEWVANIKQDGSEKYYVDSVKGRFTISRDNAKNSLYLQMNSLRAE |
| | VH-VL | DTAVYYCATGSFDYWGQGTLVTVSSGGGGSGGGGSGGGGSGGG |
| | | GSQPVLTQPSSHSASSGASVRLTCMLSSGFSVGDFWIRWYQQKPG |
| | | NPPRYLLFYHSDSNKGQGSGVPSRFSGSNDASANAGILRISGLQPE |
| | | DEADYYCGTWHSNSKTWVFGCGTKLTVL |
| 1170 | ABL-CD3- | EVQLVESGGGLVQPGGSLRLSCAASGFTFSSYWMSWVRQAPGKC |
| | 45A_scFv_ | LEWVANIKEDGSEKYYVDSVKGRFTISRDNAKNSLYLQMNSLRAE |
| | VH-VL | DTAVYYCARERRTGYLDYWGQGTLVTVSSGGGGSGGGGSGGGG |
| | | SGGGGSDIQMTQSPSTLSASVGDRVTITCRASQSISSWLAWYQQKP |
| | | GKAPNLLIYKASSLESGVPSRFSGSGSGTEFTLTISSLQPDDFATYYC |
| | | QQYKSYSWTIGCGTKVEIK |
| 1171 | ABL-CD3- | EVQLVESGGGLVQPGGSLRLSCAASGFTFSRYSMNWVRQAPGKCL |
| | 47A_scFv_ | EWVSYISRSSTTIYYADSVKGRFTISRDNAKNSLYLQMNSLRDEDA |
| | VH-VL | AVYYCARERWELLYFDYWGQGTLVTVSSGGGGSGGGGSGGGGS |
| | | GGGGSNFMLTQPHSVSESPGKTVTISCTRSSGSIASKYVQWYQQRP |
| | | GSSPTTVIYEDNQRPSGVPDRFSGSIDSSSNSASLTISGLKTEDEADY |
| | | YCQSYYSNNRVFGCGTKLTVL |
| 1172 | ABL-CD3- | EVQLVESGGGLVKPGGSLRLSCSASGFTFSTYTMNWVRQAPGKCL |
| | 49A_scFv_ | EWVSSISKSSSYIYYTDSVKGRFTISRDNAKNSLYLQMNSLRAEDT |
| | VH-VL | AVYYCARERRTLAFDIWGQGTMVTVSSGGGGSGGGGSGGGGSGG |
| | | GGSNFMLTQPHSVSESPGKTVTISCTRSSGSIASNYVQWYQQRPGS |
| | | SPTTVIYEDNQRSSGVPDRFSGSIDSSSNSASLTISGLKTEDEADYYC |
| | | QSYDRSNRVFGCGTKLTVL |
| 1173 | ABL-CD3- | EVQLVESGGGLVKPGGSLRLSCAASGFTFSYYSMNWVRQAPGKC |
| | 52A_scFv_ | LEWVSSISRSTTYIYYADSVKGRFTISRDNAKNSLYLQMNSLRAED |
| | VH-VL | TAVYYCARDGKGAIFLDYWGQGTLVTVSSGGGGSGGGGSGGGGS |
| | | GGGGSSYELTQPPSVSVSPGQTARITCSGDALPKKYAYWYQQKSG |
| | | QAPVLVIYEDSKRPSGIPERFSGSSSGTMATLTISGAQVEDEADYYC |
| | | YSTDSSGNHRVFGCGTKLTVL |
| 1174 | ABL-CD3- | EVQLVESGGGLVKPGGSLRLSCAASGFTFSSYTMNWVRQAPGKCL |
| | 53A_scFv_ | EWVSSITTSSSYIYYADSVKGRFTISRDNAKNSLYLQMNSLRAEDT |
| | VH-VL | AVYYCARVTFDAFDIWGQGTMVTVSSGGGGSGGGGSGGGGSGG |
| | | GGSNFMLTQPHSVSESPGKTVTISCTRSSGSIASNYVQWYQQRPGS |
| | | SPTTVIYEDNQRPSGVPDRFSGSIDSSSNSASLTISGLKTEDEADYYC |
| | | QSYDSSNRVFGCGTKLTVL |
| 1175 | ABL-CD3- | EVQLVESGGGLVKPGGSLRLSCVASGFTFSRYSMNWVRQAPGKCL |
| | 56A_scFv_ | EWVSSISSSSRYTYYADSLKGRFIMSRDNAKNSLFLQMNSLRAEDT |
| | VH-VL | AVYYCARNLYNALDYWGQGTLVTVSSGGGGSGGGGSGGGGSGG |
| | | GGSFMLTQPHSVSESPGKTVTISCTRSSGSIASNYVQWYQQRPGSSP |
| | | TTVIYEDNQRPSGVPDRFSGSIDSSSNSASLTISGLQTEDEADYYCQ |
| | | SYDSSNRVFGCGTKLTVL |
| 1176 | ABL-CD3- | EVQLVESGGGLVKPGGSLRLSCAASGFTFSSYTMNWVRQAPGKCL |
| | 57A_scFv_ | EWVSSISTSSRYIYYTDSVKGRFTISRDDAKNSLYLQMNSLRAEDT |
| | VH-VL | AVYYCTRNWGYDVFDIWGQGTMVIVSSGGGGSGGGGSGGGGSG |
| | | GGGSQPVLTQPSSHSASSGASVRLTCMLSSGFSVGDFWIRWYQQK |
| | | PGNPPRYLLYYHSDSNKGQGSGVPSRFSGSNDASANAGILRISGLQ |
| | | PEDEADYYCGTWHSNSKTQVFGCGTKLTVL |
| 1177 | ABL-CD3- | EVQLVESGGGLVKPGGSLRLSCAASGFTFSSYSMNWVRQAPGKCL |
| | 61A_scFv_ | EWVSSISRSGTYIYYADSVKGRFTISRDNAKNSLYLQMNSLRAEDT |
| | VH-VL | AVYYCARERWELLYFDYWGQGTLVTVSSGGGGSGGGGSGGGGS |
| | | GGGGSQTVVTQEPSLTVSPGGTVTLTCASSTGAVTSGYYPNWFQQ |
| | | KPGQAPRALIYSTSNKHSWTPARFSGSLLGGKAALTLSGVQPEDEA |
| | | EYYCLLYYGGAYVFGCGTKVTVL |
| 1178 | ABL-CD3- | EVQLVESGGGLVKPGGSLRLSCAASGFTFSSYSMNWVRQAPGKCL |
| | 62A_scFv_ | EWVSSISRSSGYIFYADSVKGRFTISRDNAKNSLYLQMNSLRAEDT |
| | VH-VL | AVYYCVRERVGAGFDYWGQGTLVTVSSGGGGSGGGGSGGGGSG |
| | | GGGSNFMLTQPHSVSESPGKTVAISCTRSSGSIASNYVQWYQQRPG |
| | | SSPTAVIYEDNQRPSGVPDRFSGSIDSSSNSAFLTISGLKTEDEADYY |
| | | CQSYDSSNRVFGCGTKLTVL |
| 1179 | ABL-CD3- | QVQLQESGPGLVKPSETLSLTCTVSGGSISSYYWSWIRQPAGKCLE |
| | 63A_scFv_ | WIGRIYTSGSTKYNPSLKSRVTMSVDTSKNQFSLKLISVTAADTAV |
| | VH-VL | YFCARERIRDLYFDYWGQGTLVTVSSGGGGSGGGGSGGGGSGGG |
| | | GSNFMLTQPHSVSESPGKTVTISCTRTSGSIASNYVQWYQQRPGSS |
| | | PTTVIYEDNQRPSGVPDRFSGSIDSSSNSASLTISGLKTEDEAAYYC |
| | | QSYDRSNRVFGCGTKLTVL |
| 1180 | ABL-CD3- | EVQVAESGGGLVKPGGSLRLSCAASGFTFSRYSMNWVRQAPGKC |
| | 64A_scFv_ | LEWVSSISSSGSYTYYADSVKGRFTISRDNAKNSLYLQMNSLRAED |
| | VH-VL | TAVYYCVRERGKGAGMDVWGQGTTVTVSSGGGGSGGGGSGGGG |
| | | SGGGGSNFMLTQPHSVSESPGKTVTISCTRSSGSIASNYVQWYQQR |
| | | PGSSPTTVIYEDNQRPSGVPDRFSGSIDSSSNSASLTISGLKTEDEAD |
| | | YYCQSYDSSNRVFGCGTKLTVL |
| 1181 | ABL-CD3- | QVQLQQWGAGLLKPSETLSLTCAVYGGSFSGYYWSWIRQPPGKC |
| | 65A_scFv_ | LEWIGEIQHSGSTNYNPSLKSRVTISVDTSKNQFSLKLSSVTAADTA |
| | VH-VL | VYYCAREYYYYGMDVWGQGTTVTVSSGGGGSGGGGSGGGGSGG |
| | | GGSQSVLTQPPSASGTPGQRVTISCSGSSSNIGSNYVYWYQQLPGT |
| | | APKLLIYSNNQRPSGVPDRFSGSKSGTSASLAISGLRSEDEADYYCA |
| | | AWDDSLSGVVFGCGTKLTVL |
| 1182 | ABL-CD3- | QVQLQESGPGLVKPSETLSLTCTVSGGSISSYYWSWIRQPAGKCLE |
| | 66A_scFv_ | WIGRIYASGSTYYNPSLKSRVTMSVDTSKNQFSLKLSSVTAADTAV |
| | VH-VL | YYCAREKRSRLYFDYWGQGTLVTVSSGGGGSGGGGSGGGGSGGG |
| | | GSNFMLTQPHSVSESPGKTVTISCTRSSGSIASNYVQWYQQRPGSSP |
| | | TTVIYEDNQRPSGVPDRFSGSIDSSSNSASLTISGLKTEDEGDYYCQ |
| | | SYDRSNRVFGCGTKLTVL |
| 1183 | ABL-CD3- | EVQLVESGGGLVKPGGSLRLSCAASGFTFSSSIMNWVRQAPGKCL |
| | 68A_scFv_ | EWVSSISGSSSYIYYADSVKGRFTISRDNAKNSQYLQMKSLRAEDT |
| | VH-VL | AVYYCARERWNLLYFDYWGQGTLVTVSSGGGGSGGGGSGGGGS |
| | | GGGGSSYELTQPPSVSVSPGQTARITCSGDALPKQYAYWYQQKPG |
| | | QAPVLVIYKDSERPSGIPERFSGSSSGTTVTLTISGVQAEDEADYYC |
| | | QSADSSGTYVVFGCGTKLTVL |
| 1184 | ABL-CD3- | QVQLQESGPGLVKPSETLSLTCTVSGGSINSYYWSWIRQPAGKCLE |
| | 71A_scFv_ | WIGRIYTSGSTNYNPSLKSRVTMSVDTSKKQFSLKLSSVTAADTAV |
| | VH-VL | YYCARERISRLYFDYWGQGILVTVSSGGGGSGGGGGGGGSGGG |
| | | GSNFMLTQPHSVSESPGKTVTISCTRSSGSIASNYVQWYQQRPGSSP |
| | | TTVIYEDNQRPSGVPDRFSGSIDSSSNSASLTISGLKTEDEADYYCQ |
| | | SYDRSNRVFGCGTKLTVL |
| 1185 | ABL-CD3- | EVQLVESGGGLVKPGGSLRLSCAASGFTFSTYIMNWVRQAPGKCL |
| | 72A_scFv_ | EWVSSISSSRSYIYYADSVKGRFTISRDNAKNSLYLQMNSLRAEDT |
| | VH-VL | AVYYCAREDRYRLFFDYWGQGTLVTVSSGGGGSGGGGSGGGGSG |
| | | GGGSNFMLTQPHSVSESPGKTVTISCTRSSGSIASNYVQWYQQRPG |
| | | SSPTTVIYEDNQRPSGVPDRFSGSIDSSSNSASLTISGLKTEDEADYY |
| | | CQSYDSSNRVFGCGTKLTVL |
| 1186 | ABL-CD3- | EVQLVESGGGLVKPGGSLRLSCAASGFTFSSYSMKWVRQAPGKCL |
| | 74A_scFv_ | EWVSSISSSSSYIYYADSVKGRFTISRDNAKNSLYLQMNSLRAEDT |
| | VH-VL | AVYYCARDRGELTLWYWGQGTLVTVSSGGGGSGGGGSGGGGSG |
| | | GGGSSYELTQPPSVSVSPGQTARITCSGDALPKKYAYWYQQKSGQ |
| | | APVLVIYEDSKRPSGIPERFSGSSSGTMATLTISGAQVEDEADYYCY |
| | | STDSSGNHWVFGCGTKLTVL |
| 1187 | ABL-CD3- | EVQLVESGGGLVKPGGSLRLSCAASGFTFSNYYMNWVRQAPGKC |
| | 76A_scFv_ | LERVSSISSSSNYIYYADSVKGRFTISRDNAKNSLYLQMNSLRAEDT |
| | VH-VL | AVYYCARDRRELPLDYWGQGTVVTVSSGGGGSGGGGSGGGGSG |
| | | GGGSSYELTQPPSVSVSPGQTARITCSGDALPKKYAYWYQQKSGQ |
| | | APVLVIYEDSKRPSGIPERFSGSSSGTMATLTISGAQVEDEADYYCY |
| | | STDSSGNHWVFGCGTKLTVL |
| 1188 | ABL-CD3- | EVQLVESGGGLVKPGGSLRLSCAASGFTFSSYSMNWVRQAPGKCL |
| | 77A_scFv_ | EWVSSISSSSSYIYYADSVKGRFTISRDNAKNSLYLQMNSLRAEDT |
| | VH-VL | AVYYCARDGELGIGTYFDYWGQGTLVTVSSGGGGSGGGGSGGGG |
| | | SGGGGSQSVLTQPPSVSGAPGQRVTISCTGSSSNIGAGYDVHWYQ |
| | | QLPGTAPKLLIYGNSNRPSGVPDRFSGSKSGTSASLAITGLQAEDEA |
| | | DYYCQSYDSSLSGSVFGCGTKLTVL |
| 1189 | ABL-CD3- | EVQLVESGGGLVKPGGSLRLSCAASGFTVSNYSMNWVRQAPGKC |
| | 79A_scFv_ | LEWVSSISRSSSYIYYADSVKGRFTISRDNAKNSLFLQMNSLRAEDT |
| | VH-VL | AVYYCARERWELLYFDYWGQGTLVTVSSGGGGSGGGGSGGGGS |
| | | GGGGSQTVVTQEPSFSVSPGGTVTLTCGLSSGSVSTSYYPSWYQQT |
| | | PGQAPRTLIYSTNTRSSGVPDRFSGSILGNKAALTITGAQADDESDY |
| | | YCVLYMGSGIWVFGCGTKLTVL |
| 1190 | ABL-CD3- | QVQLQESGPGLVKPSQTLSLTCTVSGGSISSGGYYWSWIRQHPGKC |
| | 81A_scFv_ | LEWIGYIYYSGSTYYNPSLKSRVTISVDTSKDQFSLKLSSVTAADTA |
| | VH-VL | VYYCARERKTSLPFDIWGQGTMVTVSSGGGGSGGGGSGGGGSGG |
| | | GGSNFMLTQPHSVSESPGKTVTISCTRSSGSIASNYVQWYQQRPGS |
| | | SPTTVIYEDNQRPSGVPDRFSGSIDSSSNSASLTISGLKTEDEAYYYC |
| | | QSFDSSNRVFGCGTKLTVL |
| 1191 | ABL-CD3- | EVQLVESGGGLVKPGGSLRLSCAASGFTFSSYSMNWVRQAPGKCL |
| | 85A_scFv_ | EWVSSISRRSGYIYYADSVKGRFTLSRDNAKKSLYLQMNSLRAED |
| | VH-VL | TAVYYCARDLNWAFDYWGQGTLVTVSSGGGGSGGGGSGGGGSG |
| | | GGGSNFMLTQPHSVSESPGKTVTISCTRSSGSIASNYVQWYQQRPG |
| | | SSPTTVIYEDNHRPSGVPDRFSGSIDSSSNSASLTIAGLKTEDEADYY |
| | | CQSYDSSNVVFGCGTKLTVL |
| 1192 | ABL-CD3- | EVQLVESGGGLVKPGGSLRLSCAASGFTFSSYSMNWVRQAPGKCL |
| | 85B_scFv_ | EWVSSISRRSGYIYYADSVKGRFTLSRDNAKKSLYLQMNSLRAED |
| | VH-VL | TAVYYCARDLNWAFDYWGQGTLVTVSSGGGGSGGGGSGGGGSG |
| | | GGGSNFMLTQPHSVSESPGKTVTISCTRSSGSIASNYVQWYQQRPG |
| | | SSPTTVIYEDNHRPSGVPDRFSGSIDSSSNSASLTIAGLKTEDEADYY |
| | | CQSSDSSNVVFGCGTKLTVL |
| 1193 | ABL-CD3- | EVQLVESGGGLVKFGGSLRLSCAASGFTFSRYSMNWVRQAPGKCL |
| | 87A_scFv_ | EWVSSISSSSSYKYYADSVKGRFTISRDNAKNSLYLQMNSLRAEDT |
| | VH-VL | AVYYCAREGGNSHGMDVWGQGTTVTVSSGGGGSGGGGSGGGGS |
| | | GGGGSNFMLTQPHSVSESPGKTVTISCTRSSGSIASNYVQWYQQRP |
| | | GSSPTTVIYEDNQRPSGVPDRFSGSIDSSSNSASLTISGLKTEDEADY |
| | | YCQSYDSSNRVFGCGTKLTVL |
| 1194 | ABL-CD3- | EVQLVESGGGLVQPGGSLRLSCAASGFTFSRYWMSWVRQAPGKC |
| | 88A_scFv_ | LEWVANIKEDGSEKYYVDSVKGRFTISRDNAKNSLYLQMNSLRAE |
| | VH-VL | DTAVYFCARERRTGYFDYWGQGTLVTVSSGGGGSGGGGSGGGGS |
| | | GGGGSDIQMTQSPSTLSASVGDRVTITCRASQSISSWLAWYQQKPG |
| | | KAPNLLIYKASSLESGVPSRFSGSGSGTEFTLTISSLQPDDFATYYCQ |
| | | QYNSYSWTFGCGTKVEIK |
| 1195 | ABL-CD3- | EVQLVESGGGLVQPGGSLRLSCAASGFTFSSYWMSWVRQAPGKC |
| | 89A_scFv_ | LEWVSSISSRRGYKYHADSVKGRFTISRDNAKNSLYLQMNSLRAE |
| | VH-VL | DTAVYYCARVPYSGSYFDYWGQGTLVTVSSGGGGSGGGGSGGG |
| | | GSGGGGSEIVLTQSPGTLSLSPGERATLSCRASQSVSSSYLAWYQQ |
| | | KPGQAPRLLIYGASSRATGIPDRFSGSGSGTDFTLTISRLAPEDFAV |
| | | YYCQQYGSSRWSFGCGTKVEIK |
| 1196 | ABL-CD3- | DIVMTQTPLSSPVTPGQPASISCRSSQSLVHSDGNTYLNWLQQRPG |
| | 18A_scFv_ | QPPRLLIYKISDRFSGVPDRFSGSGAGTDFTLKIGRVEAEDVGVYYC |
| | VL-VH | MQSTQFPITFGCGTRLEIKGGGGSGGGGSGGGGSGGGGSEVQLVE |
| | | SGEGLVQPGGSLRLSCAASGFTFSSYAMHWVRQAPGKCLEYVSAI |
| | | SSNGGRTYYADSVKGRFTISRDNSKNTLYLQMGSLRAEDMAVYY |
| | | CARGGSLDYWGQGTLVTVSS |
| 1197 | ABL-CD3- | NFMLTQPHSVSESPGKTVTISCTRSSGSIASNYVQWYQQRPGSSPTS |
| | 20A_scFv_ | VIYEDNQRPSGVPDRFSGSIDSSSNSASLTISGLKTEDEADYYCQSY |
| | VL-VH | DRSNRVFGCGTKLTVLGGGGSGGGGSGGGGSGGGGSEVQLVESG |
| | | GGLVKPGGSLRLSCAASGLTFSTYSMNWVRQAPGKCLEWVSSISS |
| | | SGSYVYYADSVKGRFTMSRDNAKNSLYLQMNSLRAEDTAVYYCA |
| | | RERRELLFDYWGQGTLVTVSS |
| 1198 | ABL-CD3- | SYELTQPPSVSVSPGQTARITCSGDALPKKYAYWYQQKSGQAPVL |
| | 21A_scFv_ | VIYEDSKRPSGIPERFSGSSSGTMATLTISGAQVEDEADYYCYSTHS |
| | VL-VH | SGNHRVFGCGTKLTVLGGGGSGGGGSGGGGSGGGGSEVQLVESG |
| | | GGLVKPGGSLRLSCAASGFTFSSYSMNWVRQAPGKCLEWVSSISR |
| | | SSSYIYYADSVKGRFTLSRDNAKNSLYLQMNSLRGEDTAIYYCGR |
| | | DGKGAIFLDYWGQGTLVTVSS |
| 1199 | ABL-CD3- | SYELTQPPSVSVSPGQTARITCSGDALPKQYTYWYQQKPGQAPVL |
| | 22A_scFv_ | VIYKDSERPSGIPERFSGSSSGTTVTLTISGVQAEDEADYYCQSADS |
| | VL-VH | SGTYRVFGCGTKLTVLGGGGSGGGGSGGGGSGGGGSEVQLVESG |
| | | GGLVQPGGSLRLSCAASGFTFSSYSMNWVRQAPGKCLEWVSYISS |
| | | SSSTIYYADSVKGRFTISRDNAKNSLYLQMNSLRDEDTAVYYCAR |
| | | DGPVDYGMDVWGQGTTVTVSS |
| 1200 | ABL-CD3- | QTVVTQEPSFSVSPGGTVTLTCGLNSGSVSTSYYPSWYQQTPGQTP |
| | 27A_scFv_ | RTLIYSTNTRSSGVPDRFSGSILGNKAALTITGAQADDESDYYCVL |
| | VL-VH | YMGSGIWVFGCGTKLTVLGGGGSGGGGSGGGGSGGGGSQVQLV |
| | | QSGAEVKKPGASLKVSCKASGYTFTSYYMHWVRQAPGQCLEWM |
| | | GIINPSNGNTISAQKFQGRVTMTRDTSTSTVYMEMSSLRSEDTAVY |
| | | YCVRSWGYYVMDVWGQGTTVTVSS |
| 1201 | ABL-CD3- | QSVLTQPPSASGTPGQRVTISCSGSSSNIGSNYVYWYQQLPGTAPK |
| | 29A_scFv_ | LLIYSNNQRPSGVPDRFSGSKSGTSASLAISGLRSEDEADYYCEAW |
| | VL-VH | DDSLSGPVFGCGTKLTVLGGGGSGGGGSGGGGSGGGGSEVQLVQ |
| | | SGAEVKKPGESLKISCKGSGYSFISYWIAWVRQMPGKCLEWMGIIY |
| | | PGDSETRYRPSFQGQVTISADKSINTAYLQWSSLKAADTAMYYCA |
| | | RQGAYDGFDIWGQGTMVTVSS |
| 1202 | ABL-CD3- | DIVMTQSPLSLPVTPGEPASISCRSSQSLLHSNGYNYLDWYLQKPG |
| | 31A_scFv_ | QSPQLLIYLGSNRASGVPDRFSGSGSGTDFTLKISRVEAEDVGFYYC |
| | VL-VH | MQPLQTPYTFGCGTKLEIKGGGGSGGGGSGGGGSGGGGSEVQLV |
| | | QSGAEVKKPGESLKISCKGSGYSFTNYWIGWVRQMPGKCLEWMG |
| | | IIYPADSDTTYSPSFQGQVTISADKSITTAYLQWSSLKASDTAMYYC |
| | | ARHGGSYILDAFDIWGQGTMVTVSS |
| 1203 | ABL-CD3- | NFMLTQPHSVSESPGKTVTISCTRSSGSIASNYVQWYQQRPGSSPTT |
| | 34A_scFv_ | VIYEDNQRPSGVPDRFSGSIDSSSNSASLTISGLKTEDEADYYCQSY |
| | VL-VH | DSSNRVFGCGTKLTVLGGGGGGGGSGGGGSGGGGSEVHLVESG |
| | | GGLVKPGGSLRLSCAASGFTFSRYSMNWVRQAPGKCLEWVSSISR |
| | | SSSYIYYADSVKGRFTISRDNAKNSLYLQMNSLRAEDTAVYYCAR |
| | | EPRTLGMDYWGQGILVTVSS |
| 1204 | ABL-CD3- | DIQMTQSPSTLSASVGDRVTITCRASQSISSWLAWFQQKTGKAPKL |
| | 36A_scFv_ | LIYKASSLESGVPSRFSGSGSGTEFTLTISSLQPDDFATYYCQQYISY |
| | VL-VH | SWTFGCGTKVEIKGGGGSGGGGSGGGGSGGGGSEVQLVESGGGL |
| | | VQRGGSLRLSCAASGFTFSNYWMSWVRQAPGKCLEWVANIKEDG |
| | | SEKYYVDSVKGRFTISRDNAKHSLYLQMNSLRAEDTAVYYCARER |
| | | RTGYFDYWGQGTLVTVSS |
| 1205 | ABL-CD3- | DIQMTQSPSTLSASVGDRVTITCRASQSISSWLAWYQQKPGKAPKL |
| | 37A_scFv_ | LIHKASSLESGVPSRFSGSGSGTEFTLTISSLQPDDFATYYCQQYNS |
| | VL-VH | YWTFGCGTKVEIKGGGGSGGGGSGGGGSGGGGSEVQLVESGGGL |
| | | VQPGGSLRLSCAASGFSFSSYSMNWVRQAPGKCLEWVSHIRSSTY |
| | | SIYYADSVMGRFTISRDNAKNSLYLQMNSLRDEDTAVYFCARAKT |
| | | GGDAFDIWGQGTMVTVSS |
| 1206 | ABL-CD3- | DVVMTQSPLSLPVTLGQPASISCRSSQSLVYSDGNTYLTWFQQRPG |
| | 39A_scFv_ | QSPRRLIYKVSNRDSGVPDRFSGSGSGTDFTLKISRVEAEDVEIYYC |
| | VL-VH | MQGTHWPLTFGCGTKVEIKGGGGSGGGGSGGGGSGGGGSEVQLL |
| | | ESGGGLVQPGGSLRLSCAASGFTFSYYAMSWVRQAPGKCLEWVS |
| | | AISGSGGSTYYADSVKGRFTISRDNSKNTLYLQMNSLRAEDTAVY |
| | | YCAKEGIVGARAEYFQHWGQGTLVTVSS |
| 1207 | ABL-CD3- | QSVLTQPPSASGTPGQRVTISCSGSSSNIGSNTVNWYQQFPGTAPKL |
| | 40A_scFv_ | LIYSNNQRPSGVPDRFSGSKSGTSASLAISGLQSEDEADYYCAPWD |
| | VL-VH | DSLNGVVFGCGTKLTVLGGGGSGGGGSGGGGSGGGGSEVQLLES |
| | | GGGLVQPGGSLRLSCAASGFTFSYYAMSWVRQAPGKCLEWVSAIS |
| | | GSGGSTYYADSVKGRFTISRDNSKNTLYLQMNSLRAEDTAVYYCA |
| | | KEGIVGARAEYFQHWGQGTLVTVSS |
| 1208 | ABL-CD3- | SYELTQPPSVSVSPGQTARITCSGDALPKKYAYWYQQKSGQAPVL |
| | 41A_scFv_ | VIYEDSKRPSGIPERFSGSSSGTMATLTISGAQVEDEADYYCYSTDS |
| | VL-VH | SGNHSWVFGCGTKLTVLGGGGSGGGGSGGGGSGGGGSEVQLVES |
| | | GGGLVKPGGSLRLSCAASGFTFSSYSMNWVRQAPGKCLEWVSSV |
| | | NSRGSYIYYADSVKGRFTISRDNAKDSLYLQMNSLRADDTAVYYC |
| | | ARDRRELLLDYWGQGTLVTVSS |
| 1209 | ABL-CD3- | QSALTQPASVSGSPGQSITISCTGTSSDVGSYNLVSWYQQHPGKAP |
| | 43A_scFv_ | KLMIYEDIRRPSGVSNRFSASKSGDTASLTISGLQAEDEADYYCSSY |
| | VL-VH | AGSTTFAIFGCGTKLTVLGGGGSGGGGSGGGGSGGGGSEVQLVQS |
| | | GAEVKKPGESLKISCKGSEYSFTTYWIAWVRQMPGKCLEWMGIIY |
| | | PGDSDTTYSPSFQGQVTISADKSISIAFLQWSSLKASDTAMYYCAR |
| | | QGELDAFDIWGQGTMVTVSS |
| 1210 | ABL-CD3- | QPVLTQPSSHSASSGASVRLTCMLSSGFSVGDFWIRWYQQKPGNPP |
| | 44A_scFv_ | RYLLFYHSDSNKGQGSGVPSRFSGSNDASANAGILRISGLQPEDEA |
| | VL-VH | DYYCGTWHSNSKTWVFGCGTKLTVLGGGGSGGGGSGGGGSGGG |
| | | GSEVQLVESGGGLVQPGGSLRLSCAASGFTFSTYWMSWVRQAPG |
| | | KCLEWVANIKQDGSEKYYVDSVKGRFTISRDNAKNSLYLQMNSL |
| | | RAEDTAVYYCATGSFDYWGQGTLVTVSS |
| 1211 | ABL-CD3- | DIQMTQSPSTLSASVGDRVTITCRASQSISSWLAWYQQKPGKAPNL |
| | 45A_scFv_ | LIYKASSLESGVPSRFSGSGSGTEFTLTISSLQPDDFATYYCQQYKS |
| | VL-VH | YSWTIGCGTKVEIKGGGGSGGGGSGGGGSGGGGSEVQLVESGGG |
| | | LVQPGGSLRLSCAASGFTFSSYWMSWVRQAPGKCLEWVANIKED |
| | | GSEKYYVDSVKGRFTISRDNAKNSLYLQMNSLRAEDTAVYYCAR |
| | | ERRTGYLDYWGQGTLVTVSS |
| 1212 | ABL-CD3- | NFMLTQPHSVSESPGKTVTISCTRSSGSIASKYVQWYQQRPGSSPTT |
| | 47A_scFv_ | VIYEDNQRPSGVPDRFSGSIDSSSNSASLTISGLKTEDEADYYCQSY |
| | VL-VH | YSNNRVFGCGTKLTVLGGGGSGGGGSGGGGSGGGGSEVQLVESG |
| | | GGLVQPGGSLRLSCAASGFTFSRYSMNWVRQAPGKCLEWVSYISR |
| | | SSTTIYYADSVKGRFTISRDNAKNSLYLQMNSLRDEDAAVYYCAR |
| | | ERWELLYFDYWGQGTLVTVSS |
| 1213 | ABL-CD3- | NFMLTQPHSVSESPGKTVTISCTRSSGSIASNYVQWYQQRPGSSPTT |
| | 49A_scFv_ | VIYEDNQRSSGVPDRFSGSIDSSSNSASLTISGLKTEDEADYYCQSY |
| | VL-VH | DRSNRVFGCGTKLTVLGGGGSGGGGSGGGGSGGGGSEVQLVESG |
| | | GGLVKPGGSLRLSCSASGFTFSTYTMNWVRQAPGKCLEWVSSISK |
| | | SSSYIYYTDSVKGRFTISRDNAKNSLYLQMNSLRAEDTAVYYCAR |
| | | ERRTLAFDIWGQGTMVTVSS |
| 1214 | ABL-CD3- | SYELTQPPSVSVSPGQTARITCSGDALPKKYAYWYQQKSGQAPVL |
| | 52A_scFv_ | VIYEDSKRPSGIPERFSGSSSGTMATLTISGAQVEDEADYYCYSTDS |
| | VL-VH | SGNHRVFGCGTKLTVLGGGGSGGGGSGGGGSGGGGSEVQLVESG |
| | | GGLVKPGGSLRLSCAASGFTFSYYSMNWVRQAPGKCLEWVSSISR |
| | | STTYIYYADSVKGRFTISRDNAKNSLYLQMNSLRAEDTAVYYCAR |
| | | DGKGAIFLDYWGQGTLVTVSS |
| 1215 | ABL-CD3- | NFMLTQPHSVSESPGKTVTISCTRSSGSIASNYVQWYQQRPGSSPTT |
| | 53A_scFv_ | VIYEDNQRPSGVPDRFSGSIDSSSNSASLTISGLKTEDEADYYCQSY |
| | VL-VH | DSSNRVFGCGTKLTVLGGGGSGGGGSGGGGSGGGGSEVQLVESG |
| | | GGLVKPGGSLRLSCAASGFTFSSYTMNWVRQAPGKCLEWVSSITT |
| | | SSSYIYYADSVKGRFTISRDNAKNSLYLQMNSLRAEDTAVYYCAR |
| | | VTFDAFDIWGQGTMVTVSS |
| 1216 | ABL-CD3- | FMLTQPHSVSESPGKTVTISCTRSSGSIASNYVQWYQQRPGSSPTTV |
| | 56A_scFv_ | IYEDNQRPSGVPDRFSGSIDSSSNSASLTISGLQTEDEADYYCQSYD |
| | VL-VH | SSNRVFGCGTKLTVLGGGGSGGGGSGGGGSGGGGSEVQLVESGG |
| | | GLVKPGGSLRLSCVASGFTFSRYSMNWVRQAPGKCLEWVSSISSSS |
| | | RYTYYADSLKGRFIMSRDNAKNSLFLQMNSLRAEDTAVYYCARN |
| | | LYNALDYWGQGTLVTVSS |
| 1217 | ABL-CD3- | QPVLTQPSSHSASSGASVRLTCMLSSGFSVGDFWIRWYQQKPGNPP |
| | 57A_scFv_ | RYLLYYHSDSNKGQGSGVPSRFSGSNDASANAGILRISGLQPEDEA |
| | VL-VH | DYYCGTWHSNSKTQVFGCGTKLTVLGGGGSGGGGSGGGGSGGG |
| | | GSEVQLVESGGGLVKPGGSLRLSCAASGFTFSSYTMNWVRQAPGK |
| | | CLEWVSSISTSSRYIYYTDSVKGRFTISRDDAKNSLYLQMNSLRAE |
| | | DTAVYYCTRNWGYDVFDIWGQGTMVIVSS |
| 1218 | ABL-CD3- | QTVVTQEPSLTVSPGGTVTLTCASSTGAVTSGYYPNWFQQKPGQA |
| | 61A_scFv_ | PRALIYSTSNKHSWTPARFSGSLLGGKAALTLSGVQPEDEAEYYCL |
| | VL-VH | LYYGGAYVFGCGTKVTVLGGGGSGGGGSGGGGSGGGGSEVQLV |
| | | ESGGGLVKPGGSLRLSCAASGFTFSSYSMNWVRQAPGKCLEWVSS |
| | | ISRSGTYIYYADSVKGRFTISRDNAKNSLYLQMNSLRAEDTAVYYC |
| | | ARERWELLYFDYWGQGTLVTVSS |
| 1219 | ABL-CD3- | NFMLTQPHSVSESPGKTVAISCTRSSGSIASNYVQWYQQRPGSSPT |
| | 62A_scFv_ | AVIYEDNQRPSGVPDRFSGSIDSSSNSAFLTISGLKTEDEADYYCQS |
| | VL-VH | YDSSNRVFGCGTKLTVLGGGGSGGGGSGGGGSGGGGSEVQLVES |
| | | GGGLVKPGGSLRLSCAASGFTFSSYSMNWVRQAPGKCLEWVSSIS |
| | | RSSGYIFYADSVKGRFTISRDNAKNSLYLQMNSLRAEDTAVYYCV |
| | | RERVGAGFDYWGQGTLVTVSS |
| 1220 | ABL-CD3- | NFMLTQPHSVSESPGKTVTISCTRTSGSIASNYVQWYQQRPGSSPTT |
| | 63A_scFv_ | VIYEDNQRPSGVPDRFSGSIDSSSNSASLTISGLKTEDEAAYYCQSY |
| | VL-VH | DRSNRVFGCGTKLTVLGGGGSGGGGSGGGGSGGGGSQVQLQESG |
| | | PGLVKPSETLSLTCTVSGGSISSYYWSWIRQPAGKCLEWIGRIYTSG |
| | | STKYNPSLKSRVTMSVDTSKNQFSLKLISVTAADTAVYFCARERIR |
| | | DLYFDYWGQGTLVTVSS |
| 1221 | ABL-CD3- | NFMLTQPHSVSESPGKTVTISCTRSSGSIASNYVQWYQQRPGSSPTT |
| | 64A_scFv_ | VIYEDNQRPSGVPDRFSGSIDSSSNSASLTISGLKTEDEADYYCQSY |
| | VL-VH | DSSNRVFGCGTKLTVLGGGGSGGGGSGGGGSGGGGSEVQVAESG |
| | | GGLVKPGGSLRLSCAASGFTFSRYSMNWVRQAPGKCLEWVSSISS |
| | | SGSYTYYADSVKGRFTISRDNAKNSLYLQMNSLRAEDTAVYYCVR |
| | | ERGKGAGMDVWGQGTTVTVSS |
| 1222 | ABL-CD3- | QSVLTQPPSASGTPGQRVTISCSGSSSNIGSNYVYWYQQLPGTAPK |
| | 65A_scFv_ | LLIYSNNQRPSGVPDRFSGSKSGTSASLAISGLRSEDEADYYCAAW |
| | VL-VH | DDSLSGVVFGCGTKLTVLGGGGSGGGGSGGGGSGGGGSQVQLQQ |
| | | WGAGLLKPSETLSLTCAVYGGSFSGYYWSWIRQPPGKCLEWIGEI |
| | | QHSGSTNYNPSLKSRVTISVDTSKNQFSLKLSSVTAADTAVYYCAR |
| | | EYYYYGMDVWGQGTTVTVSS |
| 1223 | ABL-CD3- | NFMLTQPHSVSESPGKTVTISCTRSSGSIASNYVQWYQQRPGSSPTT |
| | 66A_scFv_ | VIYEDNQRPSGVPDRFSGSIDSSSNSASLTISGLKTEDEGDYYCQSY |
| | VL-VH | DRSNRVFGCGTKLTVLGGGGSGGGGSGGGGSGGGGSQVQLQESG |
| | | PGLVKPSETLSLTCTVSGGSISSYYWSWIRQPAGKCLEWIGRIYASG |
| | | STYYNPSLKSRVTMSVDTSKNQFSLKLSSVTAADTAVYYCAREKR |
| | | SRLYFDYWGQGTLVTVSS |
| 1224 | ABL-CD3- | SYELTQPPSVSVSPGQTARITCSGDALPKQYAYWYQQKPGQAPVL |
| | 68A_scFv_ | VIYKDSERPSGIPERFSGSSSGTTVTLTISGVQAEDEADYYCQSADS |
| | VL-VH | SGTYVVFGCGTKLTVLGGGGSGGGGSGGGGSGGGGSEVQLVESG |
| | | GGLVKPGGSLRLSCAASGFTFSSSIMNWVRQAPGKCLEWVSSISGS |
| | | SSYIYYADSVKGRFTISRDNAKNSQYLQMKSLRAEDTAVYYCARE |
| | | RWNLLYFDYWGQGTLVTVSS |
| 1225 | ABL-CD3- | NFMLTQPHSVSESPGKTVTISCTRSSGSIASNYVQWYQQRPGSSPTT |
| | 71A_scFv_ | VIYEDNQRPSGVPDRFSGSIDSSSNSASLTISGLKTEDEADYYCQSY |
| | VL-VH | DRSNRVFGCGTKLTVLGGGGSGGGGSGGGGSGGGGSQVQLQESG |
| | | PGLVKPSETLSLTCTVSGGSINSYYWSWIRQPAGKCLEWIGRIYTSG |
| | | STNYNPSLKSRVTMSVDTSKKQFSLKLSSVTAADTAVYYCARERIS |
| | | RLYFDYWGQGILVTVSS |
| 1226 | ABL-CD3- | NFMLTQPHSVSESPGKTVTISCTRSSGSIASNYVQWYQQRPGSSPTT |
| | 72A_scFv_ | VIYEDNQRPSGVPDRFSGSIDSSSNSASLTISGLKTEDEADYYCQSY |
| | VL-VH | DSSNRVFGCGTKLTVLGGGGSGGGGSGGGGSGGGGSEVQLVESG |
| | | GGLVKPGGSLRLSCAASGFTFSTYIMNWVRQAPGKCLEWVSSISSS |
| | | RSYIYYADSVKGRFTISRDNAKNSLYLQMNSLRAEDTAVYYCARE |
| | | DRYRLFFDYWGQGTLVTVSS |
| 1227 | ABL-CD3- | SYELTQPPSVSVSPGQTARITCSGDALPKKYAYWYQQKSGQAPVL |
| | 74A_scFv_ | VIYEDSKRPSGIPERFSGSSSGTMATLTISGAQVEDEADYYCYSTDS |
| | VL-VH | SGNHWVFGCGTKLTVLGGGGSGGGGSGGGGSGGGGSEVQLVESG |
| | | GGLVKPGGSLRLSCAASGFTFSSYSMKWVRQAPGKCLEWVSSISSS |
| | | SSYIYYADSVKGRFTISRDNAKNSLYLQMNSLRAEDTAVYYCARD |
| | | RGELTLWYWGQGTLVTVSS |
| 1228 | ABL-CD3- | SYELTQPPSVSVSPGQTARITCSGDALPKKYAYWYQQKSGQAPVL |
| | 76A_scFv_ | VIYEDSKRPSGIPERFSGSSSGTMATLTISGAQVEDEADYYCYSTDS |
| | VL-VH | SGNHWVFGCGTKLTVLGGGGSGGGGSGGGGSGGGGSEVQLVESG |
| | | GGLVKPGGSLRLSCAASGFTFSNYYMNWVRQAPGKCLERVSSISS |
| | | SSNYIYYADSVKGRFTISRDNAKNSLYLQMNSLRAEDTAVYYCAR |
| | | DRRELPLDYWGQGTVVTVSS |
| 1229 | ABL-CD3- | QSVLTQPPSVSGAPGQRVTISCTGSSSNIGAGYDVHWYQQLPGTAP |
| | 77A_scFv_ | KLLIYGNSNRPSGVPDRFSGSKSGTSASLAITGLQAEDEADYYCQS |
| | VL-VH | YDSSLSGSVFGCGTKLTVLGGGGSGGGGSGGGGSGGGGSEVQLVE |
| | | SGGGLVKPGGSLRLSCAASGFTFSSYSMNWVRQAPGKCLEWVSSI |
| | | SSSSSYIYYADSVKGRFTISRDNAKNSLYLQMNSLRAEDTAVYYCA |
| | | RDGELGIGTYFDYWGQGTLVTVSS |
| 1230 | ABL-CD3- | QTVVTQEPSFSVSPGGTVTLTCGLSSGSVSTSYYPSWYQQTPGQAP |
| | 79A_scFv_ | RTLIYSTNTRSSGVPDRFSGSILGNKAALTITGAQADDESDYYCVL |
| | VL-VH | YMGSGIWVFGCGTKLTVLGGGGSGGGGSGGGGSGGGGSEVQLVE |
| | | SGGGLVKPGGSLRLSCAASGFTVSNYSMNWVRQAPGKCLEWVSSI |
| | | SRSSSYIYYADSVKGRFTISRDNAKNSLFLQMNSLRAEDTAVYYCA |
| | | RERWELLYFDYWGQGTLVTVSS |
| 1231 | ABL-CD3- | NFMLTQPHSVSESPGKTVTISCTRSSGSIASNYVQWYQQRPGSSPTT |
| | 81A_scFv_ | VIYEDNQRPSGVPDRFSGSIDSSSNSASLTISGLKTEDEAYYYCQSF |
| | VL-VH | DSSNRVFGCGTKLTVLGGGGSGGGGSGGGGSGGGGSQVQLQESG |
| | | PGLVKPSQTLSLTCTVSGGSISSGGYYWSWIRQHPGKCLEWIGYIY |
| | | YSGSTYYNPSLKSRVTISVDTSKDQFSLKLSSVTAADTAVYYCARE |
| | | RKTSLPFDIWGQGTMVTVSS |
| 1232 | ABL-CD3- | NFMLTQPHSVSESPGKTVTISCTRSSGSIASNYVQWYQQRPGSSPTT |
| | 85A_scFv_ | VIYEDNHRPSGVPDRFSGSIDSSSNSASLTIAGLKTEDEADYYCQSY |
| | VL-VH | DSSNVVFGCGTKLTVLGGGGSGGGGSGGGGSGGGGSEVQLVESG |
| | | GGLVKPGGSLRLSCAASGFTFSSYSMNWVRQAPGKCLEWVSSISR |
| | | RSGYIYYADSVKGRFTLSRDNAKKSLYLQMNSLRAEDTAVYYCA |
| | | RDLNWAFDYWGQGTLVTVSS |
| 1233 | ABL-CD3- | NFMLTQPHSVSESPGKTVTISCTRSSGSIASNYVQWYQQRPGSSPTT |
| | 85B_scFv_ | VIYEDNHRPSGVPDRFSGSIDSSSNSASLTIAGLKTEDEADYYCQSS |
| | VL-VH | DSSNVVFGCGTKLTVLGGGGSGGGGSGGGGSGGGGSEVQLVESG |
| | | GGLVKPGGSLRLSCAASGFTFSSYSMNWVRQAPGKCLEWVSSISR |
| | | RSGYIYYADSVKGRFTLSRDNAKKSLYLQMNSLRAEDTAVYYCA |
| | | RDLNWAFDYWGQGTLVTVSS |
| 1234 | ABL-CD3- | NFMLTQPHSVSESPGKTVTISCTRSSGSIASNYVQWYQQRPGSSPTT |
| | 87A_scFv_ | VIYEDNQRPSGVPDRFSGSIDSSSNSASLTISGLKTEDEADYYCQSY |
| | VL-VH | DSSNRVFGCGTKLTVLGGGGSGGGGSGGGGSGGGGSEVQLVESG |
| | | GGLVKFGGSLRLSCAASGFTFSRYSMNWVRQAPGKCLEWVSSISS |
| | | SSSYKYYADSVKGRFTISRDNAKNSLYLQMNSLRAEDTAVYYCAR |
| | | EGGNSHGMDVWGQGTTVTVSS |
| 1235 | ABL-CD3- | DIQMTQSPSTLSASVGDRVTITCRASQSISSWLAWYQQKPGKAPNL |
| | 88A_scFv_ | LIYKASSLESGVPSRFSGSGSGTEFTLTISSLQPDDFATYYCQQYNS |
| | VL-VH | YSWTFGCGTKVEIKGGGGSGGGGSGGGGSGGGGSEVQLVESGGG |
| | | LVQPGGSLRLSCAASGFTFSRYWMSWVRQAPGKCLEWVANIKED |
| | | GSEKYYVDSVKGRFTISRDNAKNSLYLQMNSLRAEDTAVYFCARE |
| | | RRTGYFDYWGQGTLVTVSS |
| 1236 | ABL-CD3- | EIVLTQSPGTLSLSPGERATLSCRASQSVSSSYLAWYQQKPGQAPR |
| | 89A_scFv_ | LLIYGASSRATGIPDRFSGSGSGTDFTLTISRLAPEDFAVYYCQQYG |
| | VL-VH | SSRWSFGCGTKVEIKGGGGSGGGGSGGGGSGGGGSEVQLVESGG |
| | | GLVQPGGSLRLSCAASGFTFSSYWMSWVRQAPGKCLEWVSSISSR |
| | | RGYKYHADSVKGRFTISRDNAKNSLYLQMNSLRAEDTAVYYCAR |
| | | VPYSGSYFDYWGQGTLVTVSS |

### DETAILED DESCRIPTION

The present disclosure relates to anti-CD3 antibodies. Applicants have used the proprietary AlivaMab® Mouse technology (See WO 2010/039900 and WO 2011/123708, incorporated by reference herein in their entirety) to generate panels of monoclonal antibodies (mAbs) that can target CD3 on T-cells. Mice were immunized with purified human CD3 epsilon/delta heterodimers and boosted with purified human CD3 epsilon/delta and cynomolgus CD3 epsilon/gamma heterodimers and human T cells. It is therefore possible that antibodies bind, CD3 epsilon alone (human and/or cynomolgus), CD3 delta alone (human and/or cynomolgus), CD3 gamma alone (human and/or cynomolgus), interfaces among these subunits, and conformation epitopes among these subunits only present in certain assemblies.

Embodiments of the disclosure pertain to the use of anti-CD3 antibodies, or antigen-binding fragments thereof, for the diagnosis, assessment and treatment of cancer and autoimmunity.

Portions of variable regions from the disclosed antibodies may include all or a combination of the complementarity determining regions (CDRs) of the VH and/or VL. The variable regions may be formatted with constant regions, either native or desirably modified for induction of either up-regulation or down-regulation of various effector functions, in a standard antibody structure (two heavy chains with two light chains). The variable regions may also be formatted as multi-specific antibodies, e.g., bispecific antibodies binding to two different epitopes on the CD3 protein or to two different antigens, one of which is CD3. In some embodiments, the antibody is a bispecific antibody. In some embodiments, the bispecific antibody comprises a tumor targeting arm. The variable regions may also be formatted as antibody fragments, e.g., single-domain antibodies comprising a single VH or VL, Fab, Fab′2, scFv, or chimeric antigen receptor (CAR). The antibodies may also be used as antibody-drug conjugates or carry other additions such as small molecule toxins or included as a part thereof, biologic toxins, radioisotopes, cytokines, oligopeptides, RNAs, or CAR-T cells to increase therapeutic modality and/or increase safety.

The practice of the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 5th ed., Wiley & Sons, 2002; Sambrook and Russell, Molecular Cloning: A Laboratory Manual (4th Edition, 2012); and other like references.

This disclosure is not limited to particular compositions or biological systems, which can vary. It is also to be understood that the terminology used herein is for the purpose of describing particular illustrative embodiments only and is not intended to be limiting. The terms used in this specification generally have their ordinary meaning in the art, within the context of this disclosure and in the specific context where each term is used. Certain terms are discussed below or elsewhere in the specification, to provide additional guidance to the practitioner in describing the compositions and methods of the disclosure and how to make and use them. The scope and meaning of any use of a term will be apparent from the specific context in which the term is used. As such, the definitions set forth herein are intended to provide illustrative guidance in ascertaining particular embodiments of the disclosure, without limitation to particular compositions or biological systems.

As used in the present disclosure and the appended claims, the singular forms “a,” “an” and “the” include plural references unless the content clearly dictates otherwise.

Throughout the present disclosure and the appended claims, unless the context requires otherwise, the word “comprise”, or variations such as “comprises” or “comprising,” will be understood to imply the inclusion of a stated element or group of elements but not the exclusion of any other element or group of elements. “Comprising” may be synonymous with “including” or “containing.”

It should be understood that wherever embodiments are described herein with the language “comprising,” otherwise analogous embodiments described in terms of “consisting of” and/or “consisting essentially of” are also provided. As used herein, “consisting of” is a closed term that includes only the specific elements recited, and “consisting essentially of” includes the specific elements recited and may include additional unrecited, nonmaterial elements.

As used herein, the term “about” modifying the quantity of an ingredient, parameter, calculation, or measurement in the compositions of the disclosure or employed in the methods of the disclosure refers to variation in the numerical quantity that can occur, for example, through typical measuring and liquid handling procedures used for making isolated antibodies or pharmaceutical compositions in the real world; through inadvertent error in these procedures; through differences in the manufacture, source, or purity of the ingredients employed to make the compositions or carry out the methods; and the like without having a substantial effect on the chemical or physical attributes of the compositions or methods of the disclosure. Such variation can be within 10%, more typically still within 5%, of a given value or range. Whether or not modified by the term “about”, the disclosure includes equivalents to the quantities. Reference to “about” a value or parameter herein includes (and describes) embodiments that are directed to that value or parameter per se. For example, description referring to “about X” includes description of “X.” Numeric ranges are inclusive of the numbers defining the range. Moreover, all ranges disclosed herein are to be understood to encompass any and all subranges subsumed therein.

The terms “antibody” (Ab) and “immunoglobulin” (Ig) are used interchangeably herein and refer to a molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the molecule. An antibody may be either membrane bound or secreted. As used herein, the term encompasses not only intact, or “whole”, polyclonal or monoclonal antibodies, but also fragments thereof (such as single-variable domain (VH, VL or combination thereof) antibodies, Fab, Fab′, F(ab′)2, Fv, single chain (ScFv), synthetic variants thereof, naturally occurring variants, fusion proteins comprising an antibody portion with an antigen-binding fragment of the required specificity, humanized antibodies, chimeric antibodies, chimeric antigen receptors (CARs), and any other modified configuration of the immunoglobulin molecule that comprises an antigen-binding site or fragment (epitope recognition site) of the required specificity.

Antibody, or Ig, molecules typically comprise two identical heavy chains and two identical light chains linked together through disulfide bonds. Both heavy chains (IgH) and light chains (IgL) contain a variable (V) region or domain and a constant (C) region or domain. The portion of the IgH locus encoding the V region comprises multiple copies of variable (V), diversity (D), and joining (J) gene segments. The portion of the IgL loci encoding the V region comprises multiple copies of V and J gene segments. The V region encoding portion of the IgH and IgL loci undergo gene segment rearrangement, e.g., different combinations of a V, (D) and J gene segments arrange to form the IgH and IgL variable regions (VH and VL, respectively), to develop diverse antigen specificity in antibodies. Each variable region comprises three hypervariable complementarity-determining regions (CDRs) interspersed between the less variable framework regions (FRs). The heavy chain comprises HCDR1, HCDR2, and HCDR3. The light chain comprises LCDR1, LCDR2, and LCDR3. The secreted form of the IgH C region of most antibodies is made up of three C domains, CH1, CH2, CH3, and a hinge region, except for Cp, which includes a CH4 regions and lacks a hinge region. The membrane-bound form of the IgH C region also has membrane and intra-cellular domains. The IgH constant region determines the isotype of the antibody, e.g. IgM, IgD, IgG1, IgG2, IgG3, IgG4, IgA and IgE. It will be appreciated that non-human mammals, such as an AlivaMab® Mouse, encoding multiple Ig isotypes will be able to undergo isotype class switching. There are two types of human IgL, Igκ and Igλ.

As used herein, the term “monoclonal antibody” or “mAb” refers to an antibody produced by an identical set of immune cells that is each a clone of a unique parent cell. Monoclonal antibodies are monospecific, have identical sequences, and bind to the same epitope in same way.

The term “antigen-binding fragment” as used herein refers to a polypeptide fragment that contains at least one CDR of an immunoglobulin heavy and/or light chain that binds to CD3. In this regard, an antigen-binding fragment of the antibodies may comprise 1, 2, 3, 4, 5, or all 6 CDRs of a VH and VL sequence set forth herein from anti-CD3 antibodies disclosed herein. In some embodiments, the antigen-binding fragment of the anti-CD3 antibodies comprise all 6 CDRs of a VH and VL sequence set forth herein from an anti-CD3 antibody disclosed herein. An antigen-binding fragment of the CD3-specific antibodies disclosed herein is capable of binding to the CD3 protein, preferably the receptor binding domain (RBD) of the CD3 protein.

In some embodiments, antibodies and antigen-binding fragments thereof disclosed herein include a heavy chain and a light chain CDR set, respectively interposed between a heavy chain and a light chain framework region (FR) set that provide conformational support to the CDRs and define the spatial relationship of the CDRs relative to each other. As used herein, the term “CDR set” refers to the three hypervariable regions of a heavy or light chain V region. Proceeding from the N terminus of a heavy or light chain, these regions are denoted as “CDR1,” “CDR2,” and “CDR3” respectively. An antigen-binding site, therefore, includes six CDRs, comprising the CDR set from each of a heavy and a light chain V region.

A “Fab” domain or fragment comprises the N-terminal portion of the IgH, which includes the V region and the CH1 domain of the IgH, and the entire IgL. A “F(ab′)₂” domain comprises the Fab domain and a portion of the hinge region, wherein the 2 IgH are linked together via disulfide linkage in the middle hinge region. Both the Fab and F(ab′)₂ are non-limiting examples of “antigen-binding fragments.”

The C-terminal portion of the IgH, which is the crystallizable fragment of an antibody following papain digestion and comprises the CH2 and CH3 domains, is referred to as the “Fc” domain. The Fc domain is the portion of the Ig recognized by cell receptors, such as the FcR, and to which the complement-activating protein, C1q, binds. The lower hinge region, which is encoded in the 5′ portion of the CH2 exon, provides flexibility within the antibody for binding to Fc receptors. Although the boundaries of the Fc domain may vary, the human IgG heavy chain Fc domain, as defined herein, comprises residue E216 to its carboxyl-terminus of the CH3 domain (or the CH4 domain for IgM and IgE antibodies), wherein the numbering is in the EU format as set forth in Edelman. The term “Fc domain” may refer to this sequence in isolation, or this sequence in the context of an antibody, antibody fragment, or Fc fusion protein. The amino acid sequence of a non-naturally occurring Fc domain (also referred to herein as a “variant Fc domain”) may comprise one or more amino acid modifications. Polymorphisms have been observed at a number of Fc domain positions, including but not limited to positions 270, 272, 312, 315, 356, and 358, according to EU numbering and, thus, slight differences between the presented sequence and sequences in the prior art may exist.

The term “EU format as set forth in Edelman” refers to the residue numbering of the human IgG1 EU antibody as described in Edelman G M et al., (1969) Proc. Natl. Acad. USA, 63, 78-85. The human IgG2 and human IgG4 residue numbering is also in the EU format (See Dillon T M, et al., J Biol Chem. June 6; 283(23):16206-15 (2008); Aalberse R C et al., Immunology 105:9-19 (2002); and Scholthauer T et al., Protein Engineering, Design and Selection, 29(10): 457-466, (2016). The EU numbering of residues may be determined by aligning an antibody at regions of homology to the sequence of the antibody with a “standard” EU numbered sequence. Unless indicated to the contrary, all residue numbering of constant regions here will be according to EU numbering.

The term “Kabat numbering” refers to the residue numbering of the variable regions as described in Kabat et al, (1991) US Department of Health and Human Services, NIH publication n 91-3242. Variable region CDRs (CDR L1, CDR L2, CDR L3, CDR H1, CDR H2, CDR H3) are identified according to contact based on crystal structures as defined in Karpusas et al. Structure. April 4; 9(4):321-9 (2001) and numbered in accordance with the Kabat numbering system. Unless indicated to the contrary, all residue numbering of variable regions will be according to Kabat.

An “Fv” fragment includes a non-covalent VH::VL heterodimer including an antigen-binding site. In certain embodiments, single chain Fv (scFv) antibodies are contemplated. A scFv is a covalently linked VH::VL heterodimer that is expressed from a gene fusion including VH- and VL-encoding genes linked by a peptide-encoding linker (see, e.g., Huston et al. (1988) Proc. Nat. Acad. Sci. USA 85(16):5879-5883 and Byrd et al (1988) Science, 242:423-6, incorporated herein by reference). As used herein, the term “linker” refers to a polypeptide sequence that joins two or more antibody domains. Linkers' characteristics and their suitability for particular purposes are known in the art. See, e.g., Chen et al. Adv Drug Deliv Rev. October 15; 65(10): 1357-1369 (2013) (disclosing various types of linkers, their properties, and associated linker designing tools and databases), which is incorporated herein by reference. Linkers may be flexible, rigid, or in vivo cleavable. Preferably, the linker is flexible. Flexible linkers typically comprise small non-polar (e.g. Gly) or polar (e.g., Ser or Thr) amino acids. The most commonly used flexible linkers have sequences consisting primarily of stretches of Gly and Ser residues (“GS” linker). Optionally, flexible linkers comprise repeats of 5 Gly and Ser residues.

Where bispecific antibodies are to be used, these may be conventional bispecific antibodies, which can be manufactured in a variety of ways (see, e.g., Holliger, P. and Winter G. Current Opinion Biotechnol. 4, 446-449 (1993); Brinkmann U, Kontermann R E. MAbs. 9(2), 182-212 (2017); Brinkmann U, Kontermann R E. Science. 372(6545), 916-917 (2021); Kontermann R E, Brinkmann U. Bispecific antibodies. Drug Discov Today. 20(7), 838-47 (2015); Spiess C, Zhai Q, Carter P J. Mol Immunol. 67(2 Pt A), 95-106 (2015); Labrijn A F, Janmaat M L, Reichert J M, Parren P W H I. Nat Rev Drug Discov. 18(8), 585-608 (2019), incorporated herein by reference), e.g., prepared chemically or from hybrid hybridomas, or may be any of the bispecific antibody fragments mentioned above.

As used herein, the term “chimeric antibody” refers to an antibody encoded by a polynucleotide sequence containing polynucleotide sequences from two or more species, e.g., human and mouse. A chimeric antibody, as used herein, may also refer to an antibody that comprises regions from two or more different antibodies.

As used herein, the term “chimeric Ig chain” refers to an Ig heavy chain or an Ig light chain encoded by a polynucleotide sequence containing polynucleotide sequences from two or more species, e.g., human and mouse. For example, a chimeric Ig heavy chain may comprise a human VH domain, DH domain, JH domain, CH1 domain, and upper hinge region and mouse CH2 and CH3 domains. In some embodiments, the middle hinge region is mouse. In some embodiments, the middle hinge region is human. In some embodiments, the middle hinge region is chimeric.

As used herein, the term “human antibody” refers to an antibody having variable and constant regions derived from human germline immunoglobulin sequences. Human antibodies can include amino acid residues not encoded by human germline immunoglobulin sequences (e.g., mutations introduced by random or site-specific mutagenesis in vitro or by somatic mutation in vivo). The term “human antibody,” however, does not encompass antibodies in which the CDR sequences are derived from the germline of another mammalian species, such as a mouse, and have been grafted onto human framework sequences (i.e., humanized antibodies). The term encompasses antibodies with sequences derived from human genes, but which have been changed, e.g., to decrease possible immunogenicity, increase affinity, decrease effector function eliminate cysteines that might cause undesirable folding, etc. The term also encompasses such antibodies comprising human amino acid sequences produced recombinantly in non-human cells, which may impart a glycosylation pattern that is not typical of human cells.

As used herein, the term “isolated” antibody or antigen-binding fragment refers to an antibody or fragment that is at least partially free of the other biological molecules present in the cells used to produce it. The other biological molecules from which an isolated antibody or fragment is at least partially free include nucleic acid molecules, proteins, lipids, carbohydrates, cellular debris and culture medium. The term “isolated” antibody or fragment does not require, but does encompass, a complete absence of such other biological molecules. The term “isolated” antibody or fragment also does not refer to a complete absence of other molecules, such as water, buffers, salts or the components of pharmaceutical formulations. Thus, a molecule that is chemically synthesized or synthesized in a cell-free system will be “isolated” from its naturally associated components. A molecule may also be “isolated” using purification techniques well known in the art. Similarly, the term “isolated” polynucleotide or nucleic acid molecule, as used herein, refers to a polynucleotide of genomic, mRNA, cDNA, or synthetic origin or some combination thereof, which (1) is not associated with all or a portion of polynucleotide with which the “isolated nucleic acid” is associated with in nature, (2) is operably linked to a polynucleotide to which it is not linked in nature, or (3) does not occur in nature as part of a larger sequence. An isolated polynucleotide “comprising” a specific sequence may also include coding sequences for other proteins or immunoglobulin chains, expression regulatory sequences or vector sequences.

As used herein, the terms “polypeptide,” “peptide” or “protein” are used interchangeably herein to describe a chain of amino acids that are linked together by chemical bonds. Non-limiting examples of a polypeptide or protein include an IgH, IgL, V domain, C domain, or an antibody.

As used herein, the term “amino acid modification” refers to at least one amino acid substitution, insertion, deletion or mutation in an amino acid sequence compared to a wild-type amino acid sequence. Such modifications are within the ordinary skill of an artisan. Some modifications, including amino acid deletions, substitutions and additions, of the Fc domain have been shown to alter the Fc domain's binding to its ligands and/or receptors resulting in a concomitant modification of effector function (see, e.g., Shields et al., J Biol Chem 276:6591-6604 (2001); Presta et al., Biochem Soc Trans 30:487-490 (2002); Escobar-Cabrera E et al. Antibodies. 6: 7 (2017); Duncan A R et al. Nature. 1988; 332: 738-740 (1988); Duncan A R et al. Nature. 332: 563-564 (1988); Hezareh M et al. J Virol. 75: 12161-12168 (2001); Oganesyan V et al. Acta Crystallogr D Biol Crystallogr; 64: 700-704 (2008); Schlothauer T et al. Protein Eng Des Sel. October; 29(10):457-466 (2016); Tao M H et al. J. Immunol. 143: 2595-2601 (1989); Von Kreudenstein T S et al. MAbs. 5(5):646-654 (2013); Wang X et al. Protein Cell. 9(1):63-73 (2018); U.S. Patent Publications 20040132101; 20070111260; 20110287032; 20180194860; U.S. Pat. No. 8,409,568; International Publication No. WO2017/177337, each incorporated by reference in its entirety). An amino acid deletion is indicated with a “A”, and an insertion is indicated with a “In”. For example, a deletion of the amino acid sequence from E216 to E222 is indicated as ΔE216-E222. An insertion of an arginine (R) between amino acid residues 234 and 235, e.g., would be indicated as InR234/235.

A “conservative amino acid substitution” replaces an amino acid residue with a different amino acid residue having similar biochemical properties (e.g., charge, hydrophobicity, or size). Generally, conservative amino acid substitutions do not substantially change the functional properties of a protein. When comparing proteins comprising conservative substitutions, the percent sequence similarity may be adjusted to account for the conservative nature of the substitution. Such adjustments are well-known in the art. See, e.g., Pearson, Methods Mol. Biol. 243:307-31 (1994). Table 2, infra, provides some of the biochemical properties of the twenty naturally occurring amino acids. Table 3, infra, provides non-limiting examples of conservative amino acid substitutions for each of the naturally occurring amino acids.

TABLE 2

| Three Letter Code | Charge | Polarity |
|---|---|---|
| Ala | neutral | nonpolar |
| Arg | positive | polar |
| Asn | neutral | polar |
| Asp | negative | polar |
| Cys | neutral | polar |
| Gly | neutral | nonpolar |
| Gln | neutral | polar |
| Glu | negative | polar |
| His | positive | polar |
| Ile | neutral | nonpolar |
| Leu | neutral | nonpolar |
| Lys | positive | polar |
| Met | neutral | nonpolar |
| Phe | neutral | nonpolar |
| Pro | neutral | nonpolar |
| Ser | neutral | polar |
| Thr | neutral | polar |
| Trp | neutral | nonpolar |
| Tyr | neutral | polar |

TABLE 3

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

The term “percent sequence identity” in the context of polypeptide (or polynucleotide) sequences is defined as the percentage of amino acid (or nucleic acid) residues in a candidate sequence that are identical with the amino acids (or nucleic acid residues) in the reference polypeptide (or polynucleotide) sequence, after aligning the sequences and introducing gaps, if necessary, to achieve the maximum percent sequence identity, and not counting any conservative substitutions as different. Such conservative substitutions are considered (in addition to identical residues) in calculating the “percent sequence similarity” of two sequences. Residue positions that are not identical but are similar differ by conservative amino acid substitutions.

Sequence alignments (e.g. for determining percent amino acid sequence identity or sequence similarity, such as between a wild type protein and a mutein thereof) can be achieved in various ways that are within the skill in the art, for instance, using publicly available sequence analysis computer software such as BLAST, BLAST-2, ALIGN, Megalign (DNASTAR), Gap, BESTFIT®, and other programs in programs in Wisconsin Package Version 10.0 or Genetics Computer Group (GCG), Madison, Wisconsin, software. The skilled artisan can determine appropriate parameters for aligning sequences, including any algorithms needed to achieve maximal alignment over the full length of the sequences being compared. Polypeptide sequences also can be compared using FASTA using default or recommended parameters. In the context of polypeptide sequences, FASTA takes the query amino acid sequence and searches a sequence database using local sequence alignment to identify similar sequences within the database (Pearson, Methods Enzymol. 183:63-98 (1990); Pearson, Methods Mol. Biol. 132:185-219 (2000); Pearson Curr Protoc Bioinformatics. March 24; 53:3.9.1-25 (2016); each herein incorporated by reference). BLAST, especially blastp or tblastn, using default parameters may be used to compare a query sequence to a database containing sequences from different organisms. See, e.g., Altschul et al., J. Mol. Biol. 215:403-410 (1990); Altschul et al., Nucleic Acids Res. 25:3389-402 (1997); Eser et al., PLoS One. 22; 9(12): e115445 (2014), each herein incorporated by reference.

Amino acids may be grouped based on their similar biochemical properties. Such groupings that may be used to define conservative substitutions include 1) amino acid residues with aliphatic side chains: glycine, alanine, valine, leucine, and isoleucine; 2) amino acid residues with aliphatic-hydroxyl side chains: serine and threonine; 3) amino acid residues with amide-containing side chains: asparagine and glutamine; 4) amino acid residues with aromatic side chains: phenylalanine, tyrosine, and tryptophan; 5) amino acid residues with basic side chains: lysine, arginine, and histidine; 6) amino acid residues with acidic side chains: aspartic acid and glutamic acid; and 7) amino acid residues with sulfur-containing side chains: cysteine and methionine. Non-limiting examples of preferred conservative amino acids substitution groups include: valine-leucine-isoleucine, phenylalanine-tyrosine, lysine-arginine, alanine-valine, glutamate-aspartate, and asparagine-glutamine.

As used herein, the term “effector function” refers to the responses of cells of the immune system that are triggered by the interaction of antibodies and antibody-antigen complexes. These effector functions usually involve one of three major mechanisms: antibody-dependent cell-mediated cytotoxicity (ADCC), complement-dependent cytotoxicity (CDC), and opsonization and phagocytosis. In ADCC, the Fc receptors on cytotoxic T cells, natural killer (NK) cells, or macrophages bind to the Fc domains of antibodies that are bound to a target cell, resulting in the secretion of substances, such as lytic enzymes, perforin, granzymes and tumor necrosis factor, that mediate the destruction of the target cell. In CDC, cell death is induced via activation of the complement cascade. See Daeron, Annu. Rev. Immunol., 15:203-234 (1997); Ward and Ghetie, Therapeutic Immunol., 2:77-94 (1995); and Ravetch and Kinet, Annu. Rev. Immunol. 9:457-492 (1991)). In opsonization and phagocytosis, a pathogen-bound antibody's Fc domain binds to a Fc receptor on the surface of a phagocyte, inducing phagocytosis. Such effector functions generally require the Fc domain to be combined with a binding domain (e.g. an antibody variable domain) and can be assessed using standard assays that are known in the art (see, e.g., WO 05/018572, WO 05/003175, and U.S. Pat. No. 6,242,195). The Fc domain of the antibody mediates immune effector mechanisms. IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. These responses can provoke unwanted side effects such as inflammation and thrombosis. Accordingly, the present disclosure further relates to anti-CD3 antibodies, with modified effector functions, including antibodies in which one or more effector functions is reduced or eliminated.

As used herein, the term “modified effector functions” refers to a Fc domain with one or more effector functions that differ from a wild-type immunoglobulin Fc domain. In some embodiments, one or more effector functions are reduced. Optionally, one or more effector functions are eliminated. The modified or reduced effector functions may be the result of lower binding affinity of the Fc domain of the antibodies disclosed herein to effector molecules (e.g., FcγRs and/or C1q). For example, the anti-CD3 antibodies disclosed herein may have reduced Fc receptor binding and complement activation compared with that of wild-type anti-CD3 antibodies. In some embodiments, a variant Fc domain has a reduced antibody dependent cell-mediated cytotoxicity (ADCC). Effector function of an anti-CD3 antibody may be determined using one of many known assays, including the CDC assay, the ADCC assay, and the phagocytosis assay (see, Xu-Rong Jiang et al., Nature Reviews Drug Discovery 10: 101-111 (2011) and Liu et al., The Journal of Biological Chemistry 292:1876-1883 (2017)). One or more of the antibody's effector functions may be reduced by at least 10%, 20%, 30%, 40%, 50%, 60%, 70%, 80%, 90%, or 95% relative to the effector function of a wild-type antibody.

The strength, or affinity of immunological binding interactions can be expressed in terms of the dissociation constant (K_{D}) of the interaction, wherein a smaller K_{D} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (kₒₙ) and the “off rate constant” (k_{off}) can be determined by calculation of the concentrations and the actual rates of association and dissociation. The ratio of k_{off}/kₒₙ enables cancellation of all parameters not related to affinity, and is thus equal to the dissociation constant, K_{D}. See, generally, Davies et al. (1990) Annual Rev. Biochem. 59:439-473. An antibody is considered to specifically bind an antigen when the K_{D} is ≤1 μM, preferably ≤100 nM. High affinity antibodies generally have a K_{D} in the low nanomolar (10⁻⁹) range, and very high affinity antibodies generally have a K_{D} in picomolar (10⁻¹²) range. A K_{D} binding affinity constant can be measured by surface plasmon resonance, e.g. using the BIACORE® system (Cytiva Life Sciences, Uppsala, Sweden and Piscataway, N.J.). See also, Jonsson et al., Ann. Biol. Clin. 51:19-26 (1993); Jonsson et al., Biotechniques 11:620-627 (1991); Jonsson et al., J. Mol. Recognit. 8:125-131 (1995); Johnsson et al., Anal. Biochem. 198:268-277 (1991); Hearty S et al., Methods Mol Biol. 907:411-42 (2012), each incorporated herein by reference. The K_{D} may also be measured using a KINEXA® system (Sapidyne Instruments, Hanover, Germany and Boise, ID). The K_{D} may also be measured by biolayer interferometry (BLI) using an OCTET® system (Sartorius AG, Goettingen, Germany). An antibody, or antigen-binding fragment thereof, is said to “specifically bind,” “immunologically bind,” and/or is “immunologically reactive” to CD3 if it reacts at a detectable level (within, for example, an ELISA assay) with CD3, and does not react detectably with unrelated polypeptides under similar conditions. In some embodiments, the antibody, or antigen-binding fragment thereof, specifically binds CD3.

As used herein, the term “avidity” refers to the overall strength of an antibody-antigen complex. Avidity relates to three major parameters: the affinity of the antibody for the epitope; the valency of both the antibody and antigen; and the structural arrangement of the parts that interact. As used herein, “avidity” describes the increased affinity that occurs as result of multiple antigen binding sites on an immunoglobulin.

As used herein, the terms “polynucleotide” and “nucleic acid molecule” are used interchangeably and refer to a polymer of nucleic acids that are linked together by chemical bonds. Polynucleotides include, but are not limited to, DNA, cDNA, RNA, mRNA, and gene sequences and segments. Polynucleotides may be isolated from a living source such as a eukaryotic cell, prokaryotic cell or virus, or may be derived through in vitro manipulation by using standard techniques of molecular biology, or by DNA synthesis, or by a combination of a number of techniques. Polynucleotides may comprise ribonucleotides, deoxynucleotides, modified forms of either type of nucleotide, or a combination thereof. A polynucleotide may be single-stranded or double-stranded. A reference herein to a nucleotide sequence encompasses its complement, unless otherwise specified. Thus, a reference to a polynucleotide having a particular sequence should be understood to encompass its complementary strand, with its complementary sequence.

As used herein, the term “highly stringent conditions” refers to hybridization to filter-bound DNA in 0.1× sodium chloride/sodium citrate (SSC) at 65° C., followed by one or more washes in 0.1×SSC, 0.1% SDS at 50-65° C. (Ausubel et al., eds., 1989, Current Protocols in Molecular Biology, Vol. I, Green Publishing Associates, Inc., and John Wiley & Sons, Inc., N.Y., at p. 2.10.3).

As used herein, the term “operably linked” refers to two structures that have been placed in a functional relationship with each other. In the context of an antibody, for example, a targeting structure may be operably linked to a structure that confers effector function. For example, the antigen-binding sequence of an antibody (e.g., variable region or VH or VL domain) may be operatively linked to a Fc domain. In the context of a polynucleotide, a coding sequence may be operatively linked to a non-coding regulatory sequence, such as a promoter, an enhance, a signal sequence, a ribosome binding sequence, a splice acceptor sequence, a splice donor sequence, a termination sequence, etc. Two operably linked structures may be directly connected. Alternatively, two operably linked structures may be connected via one or more intermediary structures. For example, the antigen-binding portion of an antibody may be operably linked to the Fc domain via a CH1 domain, a hinge region and/or a linker sequence. Similarly, operably linked non-coding regulatory sequences include both sequences that act in cis and are contiguous with the coding sequence and sequences that act in trans or at a distance to control the coding sequence.

As used herein, the term “vector” refers to a polynucleotide molecule that can replicate in a cell into which another polynucleotide fragment can be integrated without loss of the vector's ability to replicate. Vectors may originate from a virus, a plasmid or the cell of a higher organism. Vectors are utilized to introduce foreign or recombinant DNA into a host cell, wherein the vector is replicated. Non-limiting examples of vectors include viral vectors, naked DNA or RNA expression vectors, bacterial vectors, mammalian vectors, plasmids, cosmids, phage vectors, DNA or RNA expression vectors associated with cationic condensing agents, DNA or RNA expression vectors encapsulated in liposomes, and certain eukaryotic cells, such as producer cells. In some embodiments, the vectors autonomously replicate in the host cell into which they are introduced. Optionally, the vectors integrate into the host cell's genome and are replicated along with the host genome. Vectors may be capable of directing the expression of coding sequences contained within them. Such vectors are referred to herein as “recombinant expression vectors” or “expression vectors.”

A polynucleotide can be contained in a vector, which can facilitate manipulation of the polynucleotide, including introduction of the polynucleotide into a target cell. The vector may be a cloning vector, which is useful for maintaining the polynucleotide. Where the polynucleotide encodes an RNA, for expressing the encoded RNA in a particular cell, either for subsequent translation of the RNA into a polypeptide or for subsequent trans regulatory activity by the RNA in the cell, the vector may be an expression vector, which contains, in addition to the polynucleotide, regulatory elements useful for expressing the polynucleotide. An expression vector may contain the expression elements necessary to achieve, for example, sustained transcription of the encoding polynucleotide, or the regulatory elements can be operatively linked to the polynucleotide prior to its being cloned into the vector.

An expression vector generally contains a promoter sequence, which can provide constitutive or, if desired, inducible, tissue-specific or developmental-stage-specific expression of the encoding polynucleotide; a poly-A sequence; and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, alpha virus and adeno-associated virus vectors, are well known and can be purchased from a commercial source (Promega, Madison Wis.; Agilent technologies, Santa Clara Cal.; Thermo Fisher Scientific, Waltham, MA.) or can be constructed by one skilled in the art (see, e.g., Meth. Enzymol., Vol. 185, Goeddel, ed. (Academic Press, Inc., 1990); Jolly, Canc. Gene Ther. 1:51-64, 1994; Flotte, J. Bioenerg. Biomemb 25:37-42, 1993; Kirshenbaum et al., J. Clin. Invest, 92:381-387, 1993; each of which is incorporated herein by reference).

The term “polynucleotide construct” as used herein refers to a sequence of DNA artificially constructed by genetic engineering, recombineering or synthesis. In some embodiments, the DNA constructs are linearized prior to recombination. In another embodiment, the DNA constructs are not linearized prior to recombination.

As used herein, the term “host cell” refers to a cell into which a polynucleotide or a vector has been introduced. It should be understood that a “host cell” includes not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein, provided they still contain the vector, polynucleotide, or a portion thereof, either maintained episomally or integrated into the host-cell genome.

The terms “patient,” “subject,” and “individual” are used interchangeably herein and refer to either a human or a non-human animal in need to treatment. These terms include mammals, such as humans, primates (e.g., monkey), livestock such as cattle, sheep, pigs, horses and goats, and companion animals such as dogs and cats. Optionally, the subject is a human. In some embodiments, the subject is suffering from or at risk for cancer.

The terms “treating” and “treatment” with regard to a subject, refers to improving at least one symptom of the subject's disease or disorder. Treating includes curing, improving, or at least partially ameliorating the disease or disorder or any symptom of the disease or disorder. With regard to cancer, these terms refer to an increased life expectancy of an individual suffering from cancer or that one or more of the symptoms of the cancer will be reduced. The terms also encompass a decreased risk of malignancy in a subject suffering from cancer. With respect to the treatment of autoimmune disease, treatment may refer to dampening the body's immune responses and controlling the autoimmune reaction.

As used herein, the terms “prevent,” “preventing,” and “prevention” refer to the prevention or delay of the recurrence or onset of, or a reduction in one or more symptoms of a disease or a disorder in a subject as a result of the administration of an anti-CD3 antibody of the disclosure. For example, in the context of cancer, “prevent,” “preventing,” and “prevention” refer to the inhibition, reduction, delay in the development, the prevention or delay of the recurrence, onset, or development of one or more symptoms associated with cancer in a subject and the maintenance of remission in the subject. Also, in the context of an autoimmune disease, “prevent”, “preventing” and “prevention” refer to the inhibition, reduction, or delay in the development or onset of an autoimmune response or the prevention or delay of the recurrence, onset, or development of one or more symptoms associated with an autoimmune response.

As used herein, the term “therapeutically effective amount” refers to that amount of the therapeutic agent being administered, as a single agent or in combination with one or more additional agents, which will relieve to some extent one or more of the symptoms of the condition being treated. With respect to the treatment of cancer, a therapeutically effective amount refers to that amount which has at least one of the following effects: palliate, ameliorate, stabilize, reverse, prevent, slow or delay the progression of (and/or symptoms associated with) of cancer. The effective amounts that may be used in the present disclosure varies depending upon the manner of administration, the age, body weight, and general health of the subject. The appropriate amount and dosage regimen can be determined using routine skill in the art.

A “prophylactically effective amount” refers to an amount effective, at dosages and for periods of time necessary, to achieve the desired prophylactic result. Typically, since a prophylactic dose is used in subjects in remission, the prophylactically effective amount may be less than the therapeutically effective amount.

As used herein, the terms “pharmaceutically acceptable carrier” and “pharmaceutically acceptable excipient” are used interchangeably and refer to any and all solvents, dispersion media, coatings, antibacterial and antifungal agents, isotonic and absorption delaying agents, and the like that are physiologically compatible. Pharmaceutically acceptable carriers are well known in the art. See, e.g., Remington's Pharmaceutical Sciences and U.S. Pharmacopeia: National Formulary, Mack Publishing Company, Easton, PA (1984), incorporated herein by reference. These carriers, without limitation, may be included in the FDA compendium of excipients that are generally regarded as safe (GRAS) (https://www.fda.gov/food/food-ingredients-packaging/generally-recognized-safe-gras). Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Pharmaceutical compositions may be prepared by mixing an antibody disclosed herein with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY; each incorporated herein by reference).

The term “administering,” as used herein, refers to any mode of transferring, delivering, introducing, or transporting a pharmaceutical composition or other agent, such as an anti-CD3 antibody, to a subject. For cancer therapy, such administrations are typically parenteral, such as intravenous.

Each embodiment in this specification is to be applied mutatis mutandis to every other embodiment unless expressly stated otherwise.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

### Anti-CD3 Antibodies

AlivaMab® Mouse anti-CD3 antibodies were generated using both AlivaMab® XKL Mouse (a cross between AlivaMab® Kappa-Lambda mice and another genetic background) and AlivaMab® Mouse Lambda Only mice. Antibodies produced by the AlivaMab® XKL Mouse comprise a chimeric immunoglobulin heavy (IgH) chain and a human immunoglobulin kappa (Igκ) light chain or human immunoglobulin lambda (Igλ) light chain. Antibodies produced by AlivaMab® Lambda Only Mice comprise a chimeric IgH chain and a human immunoglobulin lambda (Igλ) light chain. The chimeric IgH chain of the AlivaMab® Mouse antibodies comprises a human variable region comprising a human variable heavy (VH) domain, a human diversity heavy (DH) domain, and a human joining heavy (JH) domain, a human constant heavy 1 (CH1) domain, a human upper hinge region (except for Cμ, which is naturally missing an upper hinge region), a mouse middle hinge region, a mouse CH2 domain, and a mouse CH3 domain. The human heavy chain variable region of any of the chimeric anti-CD3 antibodies may be readily appended to a fully human constant region, while maintaining the antigen-binding characteristics of the parent chimeric antibody that were developed in vivo in the AlivaMab® Mouse. In some embodiments, the human heavy chain variable region, CH1 and, optionally, upper hinge region of the chimeric antibody are appended to human hinge, a human CH2 domain and a human CH3 domain in order to produce a fully human antibody.

A first aspect of the disclosure provides an anti-CD3 antibody or an antigen-binding fragment thereof. An anti-CD3 antibody, or an antigen-binding fragment thereof, may be human. In some embodiments, an anti-CD3 antibody, or an antigen-binding fragment thereof, of the disclosure is chimeric. In some embodiments, the chimeric anti-CD3 antibody, or an antigen-binding fragment thereof, comprises a chimeric IgH chain and a human Igκ chain. In some embodiments, the chimeric anti-CD3 antibody, or an antigen-binding fragment thereof, comprises a chimeric IgH chain and a human Igλ chain. In some embodiments, the chimeric anti-CD3 antibody comprises human and mouse sequences. In some embodiments, an anti-CD3 antibody, or an antigen-binding fragment thereof, of the disclosure is a bispecific antibody. In some embodiments, the bispecific antibody further comprises a tumor-targeting arm.

In some embodiments, the anti-CD3 antibody or the antigen-binding fragment thereof comprises one to six complementarity determining regions (CDRs) disclosed herein. The one to six CDRs may comprise an amino acid sequence selected from the group consisting of SEQ ID NOs: 1-687.

In some embodiments, the anti-CD3 antibody, or antigen-binding fragment thereof, comprises a heavy chain CDR3 (HCDR3) disclosed herein. The HCDR3 may comprise an amino acid sequence selected from the group consisting of SEQ ID NOs: 574-687. In some embodiments, the anti-CD3 antibody, or antigen-binding fragment thereof, comprises a light chain CDR3 (LCDR3) disclosed herein. The LCDR3 may comprise an amino acid sequence selected from the group consisting of SEQ ID NOs: 227-344.

In some embodiments, the CDRs of an anti-CD3 antibody, or antigen-binding fragment thereof, may be mixed and matched between the CDRs of antibody clones disclosed herein. In some embodiments, an anti-CD3 antibody, or antigen-binding fragment thereof, comprises a HCDR1 comprising any HCDR1 sequence disclosed herein, a HCDR2 comprising any HCDR2 sequence disclosed herein, and a HCDR3 comprising any HCDR3 sequence disclosed herein. In some embodiments, the HCDR1, HCDR2 and HCDR3 are selected from three different anti-CD3 clones disclosed herein. In some embodiments, the HCDR1, HCDR2 and HCDR3 are selected from two different anti-CD3 clones disclosed herein. In some embodiments, the HCDR1, HCDR2, and HCDR3 are from a single anti-CD3 clone disclosed herein.

In some embodiments, an anti-CD3 antibody, or an antigen-binding fragment thereof, comprises a LCDR1 comprising any LCDR1 sequence disclosed herein, a LCDR2 comprising any LCDR2 sequence disclosed herein, and a LCDR3 comprising any LCDR3 sequence disclosed herein. In some embodiments, the LCDR1, LCDR2 and LCDR3 are selected from three different anti-CD3 clones disclosed herein. In some embodiments, the LCDR1, LCDR2 and LCDR3 are selected from two different anti-CD3 clones disclosed herein. In some embodiments, the LCDR1, LCDR2, and LCDR3 are from a single anti-CD3 clone disclosed herein. The single anti-CD3 clone may be the same anti-CD3 clone from which the HCDR1, HCDR2, and HCDR3 were selected.

In some embodiments, the six CDRs of an anti-CD3 antibody, or antigen-binding fragment thereof, are from the same anti-CD3 antibody clone disclosed herein. In some embodiments, the six CDRs of an anti-CD3 antibody, or antigen-binding fragment thereof, are selected from the corresponding VH and VL of a single clone disclosed herein. In some embodiments, an anti-CD3 antibody, or an antigen-binding fragment thereof, comprises 1) a HCDR1, a HCDR2, and a HCDR3 selected from the HCDR1, HCDR2 and HCDR3 of one VH selected from any one of the VH regions disclosed herein and 2) a LCDR1, a LCDR2, and a LCDR3 selected from the LCDR1, LCDR2 and LCDR3 of one VL selected from any one of the VL regions disclosed herein. In some embodiments, an anti-CD3 antibody, or antigen-binding fragment thereof, comprises a HCDR1, a HCDR2, a HCDR3, a LCDR1, a LCDR2, and a LCDR3 within the corresponding VH and VL amino acid sequences of a single clone as disclosed herein.

In some embodiments, an anti-CD3 antibody, or antigen-binding fragment thereof, comprises a VH comprising any one of the VH regions disclosed herein. In some embodiments, an anti-CD3 antibody, or antigen-binding fragment thereof, comprises a VL comprising any one of the VL regions disclosed herein. In some embodiments, an anti-CD3 antibody, or an antigen-binding fragment thereof, comprises a corresponding VH and VL of a single clone.

The CDRs, VH and/or VL may be selected from any one of the clones from the group consisting of ABL-CD3-1 to ABL-CD3-113, ABL-CD3-1A to ABL-CD3-113A, and ABL-CD3-85B. In some embodiments, the CDRs, VH, and/or VL may be from an antibody selected from the group consisting of ABL-CD3-18, ABL-CD3-20, ABL-CD3-21, ABL-CD3-22, ABL-CD3-27, ABL-CD3-29, ABL-CD3-31, ABL-CD3-34, ABL-CD3-36, ABL-CD3-37, ABL-CD3-39, ABL-CD3-40, ABL-CD3-41, ABL-CD3-43, ABL-CD3-44, ABL-CD3-45, ABL-CD3-47, ABL-CD3-49, ABL-CD3-52, ABL-CD3-53, ABL-CD3-56, ABL-CD3-57, ABL-CD3-61, ABL-CD3-62, ABL-CD3-63, ABL-CD3-64, ABL-CD3-65, ABL-CD3-66, ABL-CD3-68, ABL-CD3-71, ABL-CD3-72, ABL-CD3-74, ABL-CD3-76, ABL-CD3-77, ABL-CD3-79, ABL-CD3-81, ABL-CD3-85, ABL-CD3-87, ABL-CD3-88, ABL-CD3-89, ABL-CD3-18A, ABL-CD3-20A, ABL-CD3-21A, ABL-CD3-22A, ABL-CD3-27A, ABL-CD3-29A, ABL-CD3-31A, ABL-CD3-34A, ABL-CD3-36A, ABL-CD3-37A, ABL-CD3-39A, ABL-CD3-40A, ABL-CD3-41A, ABL-CD3-43A, ABL-CD3-44A, ABL-CD3-45A, ABL-CD3-47A, ABL-CD3-49A, ABL-CD3-52A, ABL-CD3-53A, ABL-CD3-56A, ABL-CD3-57A, ABL-CD3-61A, ABL-CD3-62A, ABL-CD3-63A, ABL-CD3-64A, ABL-CD3-65A, ABL-CD3-66A, ABL-CD3-68A, ABL-CD3-71A, ABL-CD3-72A, ABL-CD3-74A, ABL-CD3-76A, ABL-CD3-77A, ABL-CD3-79A, ABL-CD3-81A, ABL-CD3-85A, ABL-CD3-85B, ABL-CD3-87A, ABL-CD3-88A, and ABL-CD3-89A.

In some embodiments, the anti-CD3 antibody, or antigen-binding fragment thereof, comprises i) a heavy chain variable region comprising a HCDR1 disclosed herein, a HCDR2 disclosed herein, and a HCDR3 disclosed herein and ii) a light chain variable region comprising a LCDR1 disclosed herein, a LCDR2 disclosed herein, and a LCDR3 disclosed herein. The HCDR1 may comprise an amino acid sequence selected from the group consisting of SEQ ID NOs: 345-457. In some embodiments, the HCDR2 comprises an amino acid sequence selected from the group consisting of SEQ ID NOs: 458-573. The HCDR3 may comprise an amino acid sequence selected from the group consisting of SEQ ID NOs: 574-687. In some embodiments the LCDR1 comprises an amino acid sequence selected from the group consisting of SEQ ID NOs: 1-113. The LCDR2 may comprise an amino acid sequence selected from the group consisting of SEQ ID NOs: 114-226. In some embodiments, the LCDR3 comprises an amino acid sequence selected from the group consisting of SEQ ID NOs: 227-344. In some embodiments, the VL region comprises an amino acid sequence selected from the group consisting of SEQ ID NOs: 688-914. The VH region may comprise an amino acid sequence selected from the group consisting of SEQ ID NOs: 915-1141.

It is known in the art that during the processing and expression of Fc-containing proteins (e.g. antibody heavy chains) that the C-terminal lysine may be cleaved (also known in the art as C-terminal lysine clipping). Accordingly, for each sequence disclosed herein that contains a C-terminal lysine, the corresponding sequence without the C-terminal lysine (i.e., the C-terminal lysine cleavage product) is also contemplated. In some embodiments, the first monomer comprises a C-terminal lysine. In some embodiments, the first monomer lacks a C-terminal lysine. In some embodiments, the second monomer comprises a C-terminal lysine. In some embodiments, the second monomer lacks a C-terminal lysine.

It is also known in the art that the C-terminal cleavage process is imprecise and that additional C-terminal residues are cleaved. Accordingly, for each sequence disclosed herein that contains a C-terminal lysine, the corresponding sequence without the two C-terminal residues is also contemplated. In some embodiments, for each sequence disclosed herein that contains a C-terminal lysine, the corresponding sequence without the three C-terminal residues is also contemplated. In some embodiments, for each sequence disclosed herein that contains a C-terminal lysine, the corresponding sequence without the four C-terminal residues is also contemplated. In some embodiments, for each sequence disclosed herein that contains a C-terminal lysine, the corresponding sequence without the five C-terminal residues is also contemplated. In some embodiments, for each sequence disclosed herein that contains a C-terminal lysine, the corresponding sequence without the six C-terminal residues is also contemplated. In some embodiments, for each sequence disclosed herein that contains a C-terminal lysine, the corresponding sequence without the seven C-terminal residues is also contemplated. In some embodiments, for each sequence disclosed herein that contains a C-terminal lysine, the corresponding sequence without the eight C-terminal residues is also contemplated. In some embodiments, for each sequence disclosed herein that contains a C-terminal lysine, the corresponding sequence without the nine C-terminal residues is also contemplated. In some embodiments, for each sequence disclosed herein that contains a C-terminal lysine, the corresponding sequence without the ten C-terminal residues is also contemplated. In some embodiments, for each sequence disclosed herein that contains a C-terminal lysine, the corresponding sequence without the eleven C-terminal residues is also contemplated. In some embodiments, for each sequence disclosed herein that contains a C-terminal lysine, the corresponding sequence without the twelve C-terminal residues is also contemplated. In some embodiments, for each sequence disclosed herein that contains a C-terminal lysine, the corresponding sequence without the thirteen C-terminal residues is also contemplated. In some embodiments, for each sequence disclosed herein that contains a C-terminal lysine, the corresponding sequence without the fourteen C-terminal residues is also contemplated. In some embodiments, for each sequence disclosed herein that contains a C-terminal lysine, the corresponding sequence without the fifteen C-terminal residues is also contemplated.

In some embodiments, an anti-CD3 antibody is a whole antibody. In some embodiments, an anti-CD3 antibody is a single chain antibody. In some embodiments, an anti-CD3 antibody is a scFv. In some embodiments, an anti-CD3 antibody is a Fab. In some embodiments, an anti-CD3 antibody is a Fab′. In some embodiments, an anti-CD3 antibody is a F(ab′)₂. In some embodiments, an anti-CD3 antibody is a Fv. In some embodiments, the anti-CD3 antibody is a scFv comprising an amino acid sequence selected from the group consisting of SEQ ID NOs.: 1155-1236. The skilled artisan would recognize that an scFv fragment can be arranged, from N-terminus to C-terminus, in a VH to VL orientation or in a VL to VH orientation. As used herein, the term “scFv_VH-VL” refers to an scFv fragment which is arranged, from N-terminus to C-terminus, in a VH to VL orientation. Similarly, the term “scFv_VL-VH” refers to an scFv fragment which is arranged, from N-terminus to C-terminus, in a VL to VH orientation.

In some embodiments, the VH comprises a G44C substitution, according to Kabat numbering. In some embodiments, the VL comprises a G100C substitution, according to Kabat numbering. In some embodiments, the VH comprises a G44C substitution and the VL comprises a G100C substitution, according to Kabat numbering. In rare cases, the parental sequence at VH44 may be a different amino acid residue than G. In such instances, the VH44 residue (according to Kabat) is still mutated to cysteine. In more common, but still relatively rare cases, the parental sequence at VL position 100 may be an amino acid other than G. In such instances, the VL100 residue (according to Kabat) is still mutated to cysteine. Without being bound by theory, the introduction of such complementary cysteine residues can result in a disulfide bond, which can stabilize antibody fragments (e.g., scFv). While non-exhaustive, other Cys substitutions are known and may be employed (see Weatherill et al, PEDS (2012) 25:321-9) including VL position 99, 100a, and 101. In some embodiments, free cysteine residues in the VH and/or VL may be substituted to a non-cysteine residue.

In some embodiments, an anti-CD3 antibody is a bispecific antibody. In some embodiments, the anti-CD3 bispecific antibody further comprises a targeting antibody, or an antigen-binding fragment thereof. In some embodiments, the targeting antibody binds to a specific tissue. It is within the skill in the art to select an appropriate targeting antibody based on the tissue to be targeted. In some embodiments, the targeting antibody is an anti-tumor antibody, or an antigen-binding fragment thereof. In some embodiments, the targeting antibody binds to a specific tissue. It is within the skill in the art to select an appropriate targeting antibody based on the tumor to be targeted.

In some embodiments, the anti-CD3 antibody, or antigen-binding fragment thereof, comprises the six CDRs of antibody ABL-CD3-1 and ABL-CD3-1A. The anti-CD3 antibody, or antigen-binding fragment thereof, may comprise an LCDR1, an LCDR2, and an LCDR3 comprising the amino acid sequences of SEQ ID NOs: 1, 114, and 227, respectively; and an HCDR1, an HCDR2, and an HCDR3 comprising the amino acid sequences of SEQ ID NOs: 345, 458, and 574, respectively. In some embodiments, the anti-CD3 antibody, or antigen-binding fragment thereof, comprises the VL of antibody ABL-CD3-1. The VL may comprise the amino acid sequence of SEQ ID NO: 688. In some embodiments, the anti-CD3 antibody, or antigen-binding fragment thereof, comprises the VH of antibody ABL-CD3-1. The VH may comprise the amino acid sequence of SEQ ID NO: 915. In some embodiments, the anti-CD3 antibody, or antigen-binding fragment thereof, comprises the VL and VH of antibody ABL-CD3-1. In some embodiments, the anti-CD3 antibody, or antigen-binding fragment thereof, comprises a VL comprising the amino acid sequence of SEQ ID NO: 688 and a VH comprising the amino acid sequence of SEQ ID NO: 915. In some embodiments, the anti-CD3 antibody, or antigen-binding fragment thereof, comprises the VL of antibody ABL-CD3-1A. The VL may comprise the amino acid sequence of SEQ ID NO: 801. In some embodiments, the anti-CD3 antibody, or antigen-binding fragment thereof, comprises the VH of antibody ABL-CD3-1A. The VH may comprise the amino acid sequence of SEQ ID NO: 1028. In some embodiments, the anti-CD3 antibody, or antigen-binding fragment thereof, comprises the VL and VH of antibody ABL-CD3-1A. In some embodiments, the anti-CD3 antibody, or antigen-binding fragment thereof, comprises a VL comprising the amino acid sequence of SEQ ID NO: 801 and a VH comprising the amino acid sequence of SEQ ID NO: 1028. In some embodiments, the anti-CD3 antibody, or antigen-binding fragment thereof, comprises ABL-CD3-1_scFv_VH-VL. In some embodiments, the anti-CD3 antibody, or antigen-binding fragment thereof, comprises, from N-terminus to C-terminus, a VH comprising the amino acid sequence of SEQ ID NO: 915; a linker comprising the amino acid sequence of (Gly-Gly-Gly-Gly-Ser)n (SEQ ID NO: 1146), wherein n is 4; and a VL comprising the amino acid sequence of SEQ ID NO: 688. In some embodiments, the anti-CD3 antibody, or antigen-binding fragment thereof, comprises ABL-CD3-1_scFv_VL-VH. In some embodiments, the anti-CD3 antibody, or antigen-binding fragment thereof, comprises, from N-terminus to C-terminus, a VL comprising the amino acid sequence of SEQ ID NO: 688; a linker comprising the amino acid sequence of (Gly-Gly-Gly-Gly-Ser)n (SEQ ID NO: 1146), wherein n is 4; and a VH comprising the amino acid sequence of SEQ ID NO: 915. In some embodiments, the anti-CD3 antibody, or antigen-binding fragment thereof, comprises ABL-CD3-1A_scFv_VH-VL. In some embodiments, the anti-CD3 antibody, or antigen-binding fragment thereof, comprises, from N-terminus to C-terminus, a VH comprising the amino acid sequence of SEQ ID NO: 1028; a linker comprising the amino acid sequence of (Gly-Gly-Gly-Gly-Ser)n (SEQ ID NO: 1146), wherein n is 4; and a VL comprising the amino acid sequence of SEQ ID NO: 801. In some embodiments, the anti-CD3 antibody, or antigen-binding fragment thereof, comprises ABL-CD3-1A_scFv_VL-VH. In some embodiments, the anti-CD3 antibody, or antigen-binding fragment thereof, comprises, from N-terminus to C-terminus, a VL comprising the amino acid sequence of SEQ ID NO: 801; a linker comprising the amino acid sequence of (Gly-Gly-Gly-Gly-Ser)n (SEQ ID NO: 1146), wherein n is 4; and a VH comprising the amino acid sequence of SEQ ID NO: 1028.

In some embodiments, the anti-CD3 antibody, or antigen-binding fragment thereof, comprises the six CDRs of antibody ABL-CD3-2 and ABL-CD3-2A. The anti-CD3 antibody, or antigen-binding fragment thereof, may comprise an LCDR1, an LCDR2, and an LCDR3 comprising the amino acid sequences of SEQ ID NOs: 2, 115, and 228, respectively; and an HCDR1, an HCDR2, and an HCDR3 comprising the amino acid sequences of SEQ ID NOs: 346, 459, and 575, respectively. In some embodiments, the anti-CD3 antibody, or antigen-binding fragment thereof, comprises the VL of antibody ABL-CD3-2. The VL may comprise the amino acid sequence of SEQ ID NO: 689. In some embodiments, the anti-CD3 antibody, or antigen-binding fragment thereof, comprises the VH of antibody ABL-CD3-2. The VH may comprise the amino acid sequence of SEQ ID NO: 916. In some embodiments, the anti-CD3 antibody, or antigen-binding fragment thereof, comprises the VL and VH of antibody ABL-CD3-2. In some embodiments, the anti-CD3 antibody, or antigen-binding fragment thereof, comprises a VL comprising the amino acid sequence of SEQ ID NO: 689 and a VH comprising the amino acid sequence of SEQ ID NO: 916. In some embodiments, the anti-CD3 antibody, or antigen-binding fragment thereof, comprises the VL of antibody ABL-CD3-2A. The VL may comprise the amino acid sequence of SEQ ID NO: 802. In some embodiments, the anti-CD3 antibody, or antigen-binding fragment thereof, comprises the VH of antibody ABL-CD3-2A. The VH may comprise the amino acid sequence of SEQ ID NO: 1029. In some embodiments, the anti-CD3 antibody, or antigen-binding fragment thereof, comprises the VL and VH of antibody ABL-CD3-2A. In some embodiments, the anti-CD3 antibody, or antigen-binding fragment thereof, comprises a VL comprising the amino acid sequence of SEQ ID NO: 802 and a VH comprising the amino acid sequence of SEQ ID NO: 1029. In some embodiments, the anti-CD3 antibody, or antigen-binding fragment thereof, comprises ABL-CD3-2_scFv_VH-VL. In some embodiments, the anti-CD3 antibody, or antigen-binding fragment thereof, comprises, from N-terminus to C-terminus, a VH comprising the amino acid sequence of SEQ ID NO: 916; a linker comprising the amino acid sequence of (Gly-Gly-Gly-Gly-Ser)n (SEQ ID NO: 1146), wherein n is 4; and a VL comprising the amino acid sequence of SEQ ID NO: 689. In some embodiments, the anti-CD3 antibody, or antigen-binding fragment thereof, comprises ABL-CD3-2_scFv_VL-VH. In some embodiments, the anti-CD3 antibody, or antigen-binding fragment thereof, comprises, from N-terminus to C-terminus, a VL comprising the amino acid sequence of SEQ ID NO: 689; a linker comprising the amino acid sequence of (Gly-Gly-Gly-Gly-Ser)n (SEQ ID NO: 1146), wherein n is 4; and a VH comprising the amino acid sequence of SEQ ID NO: 916. In some embodiments, the anti-CD3 antibody, or antigen-binding fragment thereof, comprises ABL-CD3-2A_scFv_VH-VL. In some embodiments, the anti-CD3 antibody, or antigen-binding fragment thereof, comprises, from N-terminus to C-terminus, a VH comprising the amino acid sequence of SEQ ID NO: 1029; a linker comprising the amino acid sequence of (Gly-Gly-Gly-Gly-Ser)n (SEQ ID NO: 1146), wherein n is 4; and a VL comprising the amino acid sequence of SEQ ID NO: 802. In some embodiments, the anti-CD3 antibody, or antigen-binding fragment thereof, comprises ABL-CD3-2A_scFv_VL-VH. In some embodiments, the anti-CD3 antibody, or antigen-binding fragment thereof, comprises, from N-terminus to C-terminus, a VL comprising the amino acid sequence of SEQ ID NO: 802; a linker comprising the amino acid sequence of (Gly-Gly-Gly-Gly-Ser)n (SEQ ID NO: 1146), wherein n is 4; and a VH comprising the amino acid sequence of SEQ ID NO: 1029.

In some embodiments, the anti-CD3 antibody, or antigen-binding fragment thereof, comprises the six CDRs of antibody ABL-CD3-3 and ABL-CD3-3A. The anti-CD3 antibody, or antigen-binding fragment thereof, may comprise an LCDR1, an LCDR2, and an LCDR3 comprising the amino acid sequences of SEQ ID NOs: 3, 116, and 229, respectively; and an HCDR1, an HCDR2, and an HCDR3 comprising the amino acid sequences of SEQ ID NOs: 347, 460, and 576, respectively. In some embodiments, the anti-CD3 antibody, or antigen-binding fragment thereof, comprises the VL of antibody ABL-CD3-3. The VL may comprise the amino acid sequence of SEQ ID NO: 690. In some embodiments, the anti-CD3 antibody, or antigen-binding fragment thereof, comprises the VH of antibody ABL-CD3-3. The VH may comprise the amino acid sequence of SEQ ID NO:917. In some embodiments, the anti-CD3 antibody, or antigen-binding fragment thereof, comprises the VL and VH of antibody ABL-CD3-3. In some embodiments, the anti-CD3 antibody, or antigen-binding fragment thereof, comprises a VL comprising the amino acid sequence of SEQ ID NO: 690 and a VH comprising the amino acid sequence of SEQ ID NO: 917. In some embodiments, the anti-CD3 antibody, or antigen-binding fragment thereof, comprises the VL of antibody ABL-CD3-3A. The VL may comprise the amino acid sequence of SEQ ID NO: 803. In some embodiments, the anti-CD3 antibody, or antigen-binding fragment thereof, comprises the VH of antibody ABL-CD3-3A. The VH may comprise the amino acid sequence of SEQ ID NO: 1030. In some embodiments, the anti-CD3 antibody, or antigen-binding fragment thereof, comprises the VL and VH of antibody ABL-CD3-3A. In some embodiments, the anti-CD3 antibody, or antigen-binding fragment thereof, comprises a VL comprising the amino acid sequence of SEQ ID NO: 803 and a VH comprising the amino acid sequence of SEQ ID NO: 1030. In some embodiments, the anti-CD3 antibody, or antigen-binding fragment thereof, comprises ABL-CD3-3_scFv_VH-VL. In some embodiments, the anti-CD3 antibody, or antigen-binding fragment thereof, comprises, from N-terminus to C-terminus, a VH comprising the amino acid sequence of SEQ ID NO: 917; a linker comprising the amino acid sequence of (Gly-Gly-Gly-Gly-Ser)n (SEQ ID NO: 1146), wherein n is 4; and a VL comprising the amino acid sequence of SEQ ID NO: 690. In some embodiments, the anti-CD3 antibody, or antigen-binding fragment thereof, comprises ABL-CD3-3_scFv_VL-VH. In some embodiments, the anti-CD3 antibody, or antigen-binding fragment thereof, comprises, from N-terminus to C-terminus, a VL comprising the amino acid sequence of SEQ ID NO: 690; a linker comprising the amino acid sequence of (Gly-Gly-Gly-Gly-Ser)n (SEQ ID NO: 1146), wherein n is 4; and a VH comprising the amino acid sequence of SEQ ID NO: 917. In some embodiments, the anti-CD3 antibody, or antigen-binding fragment thereof, comprises ABL-CD3-3A_scFv_VH-VL. In some embodiments, the anti-CD3 antibody, or antigen-binding fragment thereof, comprises, from N-terminus to C-terminus, a VH comprising the amino acid sequence of SEQ ID NO: 1030; a linker comprising the amino acid sequence of (Gly-Gly-Gly-Gly-Ser)n (SEQ ID NO: 1146), wherein n is 4; and a VL comprising the amino acid sequence of SEQ ID NO: 803. In some embodiments, the anti-CD3 antibody, or antigen-binding fragment thereof, comprises ABL-CD3-3A_scFv_VL-VH. In some embodiments, the anti-CD3 antibody, or antigen-binding fragment thereof, comprises, from N-terminus to C-terminus, a VL comprising the amino acid sequence of SEQ ID NO: 803; a linker comprising the amino acid sequence of (Gly-Gly-Gly-Gly-Ser)n (SEQ ID NO: 1146), wherein n is 4; and a VH comprising the amino acid sequence of SEQ ID NO: 1030.

In some embodiments, the anti-CD3 antibody, or antigen-binding fragment thereof, comprises the six CDRs of antibody ABL-CD3-4 and ABL-CD3-4A. The anti-CD3 antibody, or antigen-binding fragment thereof, may comprise an LCDR1, an LCDR2, and an LCDR3 comprising the amino acid sequences of SEQ ID NOs: 4, 117, and 230, respectively; and an HCDR1, an HCDR2, and an HCDR3 comprising the amino acid sequences of SEQ ID NOs: 348, 461, and 577, respectively. In some embodiments, the anti-CD3 antibody, or antigen-binding fragment thereof, comprises the VL of antibody ABL-CD3-4. The VL may comprise the amino acid sequence of SEQ ID NO: 691. In some embodiments, the anti-CD3 antibody, or antigen-binding fragment thereof, comprises the VH of antibody ABL-CD3-4. The VH may comprise the amino acid sequence of SEQ ID NO: 918. In some embodiments, the anti-CD3 antibody, or antigen-binding fragment thereof, comprises the VL and VH of antibody ABL-CD3-4. In some embodiments, the anti-CD3 antibody, or antigen-binding fragment thereof, comprises a VL comprising the amino acid sequence of SEQ ID NO: 691 and a VH comprising the amino acid sequence of SEQ ID NO: 918. In some embodiments, the anti-CD3 antibody, or antigen-binding fragment thereof, comprises the VL of antibody ABL-CD3-4A. The VL may comprise the amino acid sequence of SEQ ID NO: 804. In some embodiments, the anti-CD3 antibody, or antigen-binding fragment thereof, comprises the VH of antibody ABL-CD3-4A. The VH may comprise the amino acid sequence of SEQ ID NO: 1031. In some embodiments, the anti-CD3 antibody, or antigen-binding fragment thereof, comprises the VL and VH of antibody ABL-CD3-4A. In some embodiments, the anti-CD3 antibody, or antigen-binding fragment thereof, comprises a VL comprising the amino acid sequence of SEQ ID NO: 804 and a VH comprising the amino acid sequence of SEQ ID NO: 1031. In some embodiments, the anti-CD3 antibody, or antigen-binding fragment thereof, comprises ABL-CD3-4_scFv_VH-VL. In some embodiments, the anti-CD3 antibody, or antigen-binding fragment thereof, comprises, from N-terminus to C-terminus, a VH comprising the amino acid sequence of SEQ ID NO: 918; a linker comprising the amino acid sequence of (Gly-Gly-Gly-Gly-Ser)n (SEQ ID NO: 1146), wherein n is 4; and a VL comprising the amino acid sequence of SEQ ID NO: 691. In some embodiments, the anti-CD3 antibody, or antigen-binding fragment thereof, comprises ABL-CD3-4_scFv_VL-VH. In some embodiments, the anti-CD3 antibody, or antigen-binding fragment thereof, comprises, from N-terminus to C-terminus, a VL comprising the amino acid sequence of SEQ ID NO: 691; a linker comprising the amino acid sequence of (Gly-Gly-Gly-Gly-Ser)n (SEQ ID NO: 1146), wherein n is 4; and a VH comprising the amino acid sequence of SEQ ID NO: 918. In some embodiments, the anti-CD3 antibody, or antigen-binding fragment thereof, comprises ABL-CD3-4A_scFv_VH-VL. In some embodiments, the anti-CD3 antibody, or antigen-binding fragment thereof, comprises, from N-terminus to C-terminus, a VH comprising the amino acid sequence of SEQ ID NO: 1031; a linker comprising the amino acid sequence of (Gly-Gly-Gly-Gly-Ser)n (SEQ ID NO: 1146), wherein n is 4; and a VL comprising the amino acid sequence of SEQ ID NO: 804. In some embodiments, the anti-CD3 antibody, or antigen-binding fragment thereof, comprises ABL-CD3-4A_scFv_VL-VH. In some embodiments, the anti-CD3 antibody, or antigen-binding fragment thereof, comprises, from N-terminus to C-terminus, a VL comprising the amino acid sequence of SEQ ID NO: 804; a linker comprising the amino acid sequence of (Gly-Gly-Gly-Gly-Ser)n (SEQ ID NO: 1146), wherein n is 4; and a VH comprising the amino acid sequence of SEQ ID NO: 1031.

In some embodiments, the anti-CD3 antibody, or antigen-binding fragment thereof, comprises the six CDRs of antibody ABL-CD3-5 and ABL-CD3-5A. The anti-CD3 antibody, or antigen-binding fragment thereof, may comprise an LCDR1, an LCDR2, and an LCDR3 comprising the amino acid sequences of SEQ ID NOs: 5, 118, and 231, respectively; and an HCDR1, an HCDR2, and an HCDR3 comprising the amino acid sequences of SEQ ID NOs: 349, 462, and 578, respectively. In some embodiments, the anti-CD3 antibody, or antigen-binding fragment thereof, comprises the VL of antibody ABL-CD3-5. The VL may comprise the amino acid sequence of SEQ ID NO: 692. In some embodiments, the anti-CD3 antibody, or antigen-binding fragment thereof, comprises the VH of antibody ABL-CD3-5. The VH may comprise the amino acid sequence of SEQ ID NO: 919. In some embodiments, the anti-CD3 antibody, or antigen-binding fragment thereof, comprises the VL and VH of antibody ABL-CD3-5. In some embodiments, the anti-CD3 antibody, or antigen-binding fragment thereof, comprises a VL comprising the amino acid sequence of SEQ ID NO: 692 and a VH comprising the amino acid sequence of SEQ ID NO: 919. In some embodiments, the anti-CD3 antibody, or antigen-binding fragment thereof, comprises the VL of antibody ABL-CD3-5A. The VL may comprise the amino acid sequence of SEQ ID NO: 805. In some embodiments, the anti-CD3 antibody, or antigen-binding fragment thereof, comprises the VH of antibody ABL-CD3-5A. The VH may comprise the amino acid sequence of SEQ ID NO: 1032. In some embodiments, the anti-CD3 antibody, or antigen-binding fragment thereof, comprises the VL and VH of antibody ABL-CD3-5A. In some embodiments, the anti-CD3 antibody, or antigen-binding fragment thereof, comprises a VL comprising the amino acid sequence of SEQ ID NO: 805 and a VH comprising the amino acid sequence of SEQ ID NO: 1032. In some embodiments, the anti-CD3 antibody, or antigen-binding fragment thereof, comprises ABL-CD3-5_scFv_VH-VL. In some embodiments, the anti-CD3 antibody, or antigen-binding fragment thereof, comprises, from N-terminus to C-terminus, a VH comprising the amino acid sequence of SEQ ID NO: 919; a linker comprising the amino acid sequence of (Gly-Gly-Gly-Gly-Ser)n (SEQ ID NO: 1146), wherein n is 4; and a VL comprising the amino acid sequence of SEQ ID NO: 692. In some embodiments, the anti-CD3 antibody, or antigen-binding fragment thereof, comprises ABL-CD3-5_scFv_VL-VH. In some embodiments, the anti-CD3 antibody, or antigen-binding fragment thereof, comprises, from N-terminus to C-terminus, a VL comprising the amino acid sequence of SEQ ID NO: 692; a linker comprising the amino acid sequence of (Gly-Gly-Gly-Gly-Ser)n (SEQ ID NO: 1146), wherein n is 4; and a VH comprising the amino acid sequence of SEQ ID NO: 919. In some embodiments, the anti-CD3 antibody, or antigen-binding fragment thereof, comprises ABL-CD3-5A_scFv_VH-VL. In some embodiments, the anti-CD3 antibody, or antigen-binding fragment thereof, comprises, from N-terminus to C-terminus, a VH comprising the amino acid sequence of SEQ ID NO: 1032; a linker comprising the amino acid sequence of (Gly-Gly-Gly-Gly-Ser)n (SEQ ID NO: 1146), wherein n is 4; and a VL comprising the amino acid sequence of SEQ ID NO: 805. In some embodiments, the anti-CD3 antibody, or antigen-binding fragment thereof, comprises ABL-CD3-5A_scFv_VL-VH. In some embodiments, the anti-CD3 antibody, or antigen-binding fragment thereof, comprises, from N-terminus to C-terminus, a VL comprising the amino acid sequence of SEQ ID NO: 805; a linker comprising the amino acid sequence of (Gly-Gly-Gly-Gly-Ser)n (SEQ ID NO: 1146), wherein n is 4; and a VH comprising the amino acid sequence of SEQ ID NO: 1032.

In some embodiments, the anti-CD3 antibody, or antigen-binding fragment thereof, comprises the six CDRs of antibody ABL-CD3-6 and ABL-CD3-6A. The anti-CD3 antibody, or antigen-binding fragment thereof, may comprise an LCDR1, an LCDR2, and an LCDR3 comprising the amino acid sequences of SEQ ID NOs: 6, 119, and 232, respectively; and an HCDR1, an HCDR2, and an HCDR3 comprising the amino acid sequences of SEQ ID NOs: 350, 463, and 579, respectively. In some embodiments, the anti-CD3 antibody, or antigen-binding fragment thereof, comprises the VL of antibody ABL-CD3-6. The VL may comprise the amino acid sequence of SEQ ID NO: 693. In some embodiments, the anti-CD3 antibody, or antigen-binding fragment thereof, comprises the VH of antibody ABL-CD3-6. The VH may comprise the amino acid sequence of SEQ ID NO: 920. In some embodiments, the anti-CD3 antibody, or antigen-binding fragment thereof, comprises the VL and VH of antibody ABL-CD3-6. In some embodiments, the anti-CD3 antibody, or antigen-binding fragment thereof, comprises a VL comprising the amino acid sequence of SEQ ID NO: 693 and a VH comprising the amino acid sequence of SEQ ID NO: 920. In some embodiments, the anti-CD3 antibody, or antigen-binding fragment thereof, comprises the VL of antibody ABL-CD3-6A. The VL may comprise the amino acid sequence of SEQ ID NO: 806. In some embodiments, the anti-CD3 antibody, or antigen-binding fragment thereof, comprises the VH of antibody ABL-CD3-6A. The VH may comprise the amino acid sequence of SEQ ID NO: 1033. In some embodiments, the anti-CD3 antibody, or antigen-binding fragment thereof, comprises the VL and VH of antibody ABL-CD3-6A. In some embodiments, the anti-CD3 antibody, or antigen-binding fragment thereof, comprises a VL comprising the amino acid sequence of SEQ ID NO: 806 and a VH comprising the amino acid sequence of SEQ ID NO: 1033. In some embodiments, the anti-CD3 antibody, or antigen-binding fragment thereof, comprises ABL-CD3-6_scFv_VH-VL. In some embodiments, the anti-CD3 antibody, or antigen-binding fragment thereof, comprises, from N-terminus to C-terminus, a VH comprising the amino acid sequence of SEQ ID NO: 920; a linker comprising the amino acid sequence of (Gly-Gly-Gly-Gly-Ser)n (SEQ ID NO: 1146), wherein n is 4; and a VL comprising the amino acid sequence of SEQ ID NO: 693. In some embodiments, the anti-CD3 antibody, or antigen-binding fragment thereof, comprises ABL-CD3-6_scFv_VL-VH. In some embodiments, the anti-CD3 antibody, or antigen-binding fragment thereof, comprises, from N-terminus to C-terminus, a VL comprising the amino acid sequence of SEQ ID NO: 693; a linker comprising the amino acid sequence of (Gly-Gly-Gly-Gly-Ser)n (SEQ ID NO: 1146), wherein n is 4; and a VH comprising the amino acid sequence of SEQ ID NO: 920. In some embodiments, the anti-CD3 antibody, or antigen-binding fragment thereof, comprises ABL-CD3-6A_scFv_VH-VL. In some embodiments, the anti-CD3 antibody, or antigen-binding fragment thereof, comprises, from N-terminus to C-terminus, a VH comprising the amino acid sequence of SEQ ID NO: 1033; a linker comprising the amino acid sequence of (Gly-Gly-Gly-Gly-Ser)n (SEQ ID NO: 1146), wherein n is 4; and a VL comprising the amino acid sequence of SEQ ID NO: 806. In some embodiments, the anti-CD3 antibody, or antigen-binding fragment thereof, comprises ABL-CD3-6A_scFv_VL-VH. In some embodiments, the anti-CD3 antibody, or antigen-binding fragment thereof, comprises, from N-terminus to C-terminus, a VL comprising the amino acid sequence of SEQ ID NO: 806; a linker comprising the amino acid sequence of (Gly-Gly-Gly-Gly-Ser)n (SEQ ID NO: 1146), wherein n is 4; and a VH comprising the amino acid sequence of SEQ ID NO: 1033.

In some embodiments, the anti-CD3 antibody, or antigen-binding fragment thereof, comprises the six CDRs of antibody ABL-CD3-7 and ABL-CD3-7A. The anti-CD3 antibody, or antigen-binding fragment thereof, may comprise an LCDR1, an LCDR2, and an LCDR3 comprising the amino acid sequences of SEQ ID NOs: 7, 120, and 233, respectively; and an HCDR1, an HCDR2, and an HCDR3 comprising the amino acid sequences of SEQ ID NOs: 351, 464, and 580, respectively. In some embodiments, the anti-CD3 antibody, or antigen-binding fragment thereof, comprises the VL of antibody ABL-CD3-7. The VL may comprise the amino acid sequence of SEQ ID NO: 694. In some embodiments, the anti-CD3 antibody, or antigen-binding fragment thereof, comprises the VH of antibody ABL-CD3-7. The VH may comprise the amino acid sequence of SEQ ID NO: 921. In some embodiments, the anti-CD3 antibody, or antigen-binding fragment thereof, comprises the VL and VH of antibody ABL-CD3-7. In some embodiments, the anti-CD3 antibody, or antigen-binding fragment thereof, comprises a VL comprising the amino acid sequence of SEQ ID NO: 694 and a VH comprising the amino acid sequence of SEQ ID NO: 921. In some embodiments, the anti-CD3 antibody, or antigen-binding fragment thereof, comprises the VL of antibody ABL-CD3-7A. The VL may comprise the amino acid sequence of SEQ ID NO: 807. In some embodiments, the anti-CD3 antibody, or antigen-binding fragment thereof, comprises the VH of antibody ABL-CD3-7A. The VH may comprise the amino acid sequence of SEQ ID NO: 1034. In some embodiments, the anti-CD3 antibody, or antigen-binding fragment thereof, comprises the VL and VH of antibody ABL-CD3-7A. In some embodiments, the anti-CD3 antibody, or antigen-binding fragment thereof, comprises a VL comprising the amino acid sequence of SEQ ID NO: 807 and a VH comprising the amino acid sequence of SEQ ID NO: 1034. In some embodiments, the anti-CD3 antibody, or antigen-binding fragment thereof, comprises ABL-CD3-7_scFv_VH-VL. In some embodiments, the anti-CD3 antibody, or antigen-binding fragment thereof, comprises, from N-terminus to C-terminus, a VH comprising the amino acid sequence of SEQ ID NO: 921; a linker comprising the amino acid sequence of (Gly-Gly-Gly-Gly-Ser)n (SEQ ID NO: 1146), wherein n is 4; and a VL comprising the amino acid sequence of SEQ ID NO: 694. In some embodiments, the anti-CD3 antibody, or antigen-binding fragment thereof, comprises ABL-CD3-7_scFv_VL-VH. In some embodiments, the anti-CD3 antibody, or antigen-binding fragment thereof, comprises, from N-terminus to C-terminus, a VL comprising the amino acid sequence of SEQ ID NO: 694; a linker comprising the amino acid sequence of (Gly-Gly-Gly-Gly-Ser)n (SEQ ID NO: 1146), wherein n is 4; and a VH comprising the amino acid sequence of SEQ ID NO: 921. In some embodiments, the anti-CD3 antibody, or antigen-binding fragment thereof, comprises ABL-CD3-7A_scFv_VH-VL. In some embodiments, the anti-CD3 antibody, or antigen-binding fragment thereof, comprises, from N-terminus to C-terminus, a VH comprising the amino acid sequence of SEQ ID NO: 1034; a linker comprising the amino acid sequence of (Gly-Gly-Gly-Gly-Ser)n (SEQ ID NO: 1146), wherein n is 4; and a VL comprising the amino acid sequence of SEQ ID NO: 807. In some embodiments, the anti-CD3 antibody, or antigen-binding fragment thereof, comprises ABL-CD3-7A_scFv_VL-VH. In some embodiments, the anti-CD3 antibody, or antigen-binding fragment thereof, comprises, from N-terminus to C-terminus, a VL comprising the amino acid sequence of SEQ ID NO: 807; a linker comprising the amino acid sequence of (Gly-Gly-Gly-Gly-Ser)n (SEQ ID NO: 1146), wherein n is 4; and a VH comprising the amino acid sequence of SEQ ID NO: 1034.

In some embodiments, the anti-CD3 antibody, or antigen-binding fragment thereof, comprises the six CDRs of antibody ABL-CD3-8 and ABL-CD3-8A. The anti-CD3 antibody, or antigen-binding fragment thereof, may comprise an LCDR1, an LCDR2, and an LCDR3 comprising the amino acid sequences of SEQ ID NOs: 8, 121, and 234, respectively; and an HCDR1, an HCDR2, and an HCDR3 comprising the amino acid sequences of SEQ ID NOs: 352, 465, and 581, respectively. In some embodiments, the anti-CD3 antibody, or antigen-binding fragment thereof, comprises the VL of antibody ABL-CD3-8. The VL may comprise the amino acid sequence of SEQ ID NO: 695. In some embodiments, the anti-CD3 antibody, or antigen-binding fragment thereof, comprises the VH of antibody ABL-CD3-8. The VH may comprise the amino acid sequence of SEQ ID NO: 922. In some embodiments, the anti-CD3 antibody, or antigen-binding fragment thereof, comprises the VL and VH of antibody ABL-CD3-8. In some embodiments, the anti-CD3 antibody, or antigen-binding fragment thereof, comprises a VL comprising the amino acid sequence of SEQ ID NO: 695 and a VH comprising the amino acid sequence of SEQ ID NO: 922. In some embodiments, the anti-CD3 antibody, or antigen-binding fragment thereof, comprises the VL of antibody ABL-CD3-8A. The VL may comprise the amino acid sequence of SEQ ID NO: 808. In some embodiments, the anti-CD3 antibody, or antigen-binding fragment thereof, comprises the VH of antibody ABL-CD3-8A. The VH may comprise the amino acid sequence of SEQ ID NO: 1035. In some embodiments, the anti-CD3 antibody, or antigen-binding fragment thereof, comprises the VL and VH of antibody ABL-CD3-8A. In some embodiments, the anti-CD3 antibody, or antigen-binding fragment thereof, comprises a VL comprising the amino acid sequence of SEQ ID NO: 808 and a VH comprising the amino acid sequence of SEQ ID NO: 1035. In some embodiments, the anti-CD3 antibody, or antigen-binding fragment thereof, comprises ABL-CD3-8_scFv_VH-VL. In some embodiments, the anti-CD3 antibody, or antigen-binding fragment thereof, comprises, from N-terminus to C-terminus, a VH comprising the amino acid sequence of SEQ ID NO: 922; a linker comprising the amino acid sequence of (Gly-Gly-Gly-Gly-Ser)n (SEQ ID NO: 1146), wherein n is 4; and a VL comprising the amino acid sequence of SEQ ID NO: 695. In some embodiments, the anti-CD3 antibody, or antigen-binding fragment thereof, comprises ABL-CD3-8_scFv_VL-VH. In some embodiments, the anti-CD3 antibody, or antigen-binding fragment thereof, comprises, from N-terminus to C-terminus, a VL comprising the amino acid sequence of SEQ ID NO: 695; a linker comprising the amino acid sequence of (Gly-Gly-Gly-Gly-Ser)n (SEQ ID NO: 1146), wherein n is 4; and a VH comprising the amino acid sequence of SEQ ID NO: 922. In some embodiments, the anti-CD3 antibody, or antigen-binding fragment thereof, comprises ABL-CD3-8A_scFv_VH-VL. In some embodiments, the anti-CD3 antibody, or antigen-binding fragment thereof, comprises, from N-terminus to C-terminus, a VH comprising the amino acid sequence of SEQ ID NO: 1035; a linker comprising the amino acid sequence of (Gly-Gly-Gly-Gly-Ser)n (SEQ ID NO: 1146), wherein n is 4; and a VL comprising the amino acid sequence of SEQ ID NO: 808. In some embodiments, the anti-CD3 antibody, or antigen-binding fragment thereof, comprises ABL-CD3-8A_scFv_VL-VH. In some embodiments, the anti-CD3 antibody, or antigen-binding fragment thereof, comprises, from N-terminus to C-terminus, a VL comprising the amino acid sequence of SEQ ID NO: 808; a linker comprising the amino acid sequence of (Gly-Gly-Gly-Gly-Ser)n (SEQ ID NO: 1146), wherein n is 4; and a VH comprising the amino acid sequence of SEQ ID NO: 1035.

In some embodiments, the anti-CD3 antibody, or antigen-binding fragment thereof, comprises the six CDRs of antibody ABL-CD3-9 and ABL-CD3-9A. The anti-CD3 antibody, or antigen-binding fragment thereof, may comprise an LCDR1, an LCDR2, and an LCDR3 comprising the amino acid sequences of SEQ ID NOs: 9, 122, and 235, respectively; and an HCDR1, an HCDR2, and an HCDR3 comprising the amino acid sequences of SEQ ID NOs: 353, 466, and 582, respectively. In some embodiments, the anti-CD3 antibody, or antigen-binding fragment thereof, comprises the VL of antibody ABL-CD3-9. The VL may comprise the amino acid sequence of SEQ ID NO: 696. In some embodiments, the anti-CD3 antibody, or antigen-binding fragment thereof, comprises the VH of antibody ABL-CD3-9. The VH may comprise the amino acid sequence of SEQ ID NO: 923. In some embodiments, the anti-CD3 antibody, or antigen-binding fragment thereof, comprises the VL and VH of antibody ABL-CD3-9. In some embodiments, the anti-CD3 antibody, or antigen-binding fragment thereof, comprises a VL comprising the amino acid sequence of SEQ ID NO: 696 and a VH comprising the amino acid sequence of SEQ ID NO: 923. In some embodiments, the anti-CD3 antibody, or antigen-binding fragment thereof, comprises the VL of antibody ABL-CD3-9A. The VL may comprise the amino acid sequence of SEQ ID NO: 809. In some embodiments, the anti-CD3 antibody, or antigen-binding fragment thereof, comprises the VH of antibody ABL-CD3-9A. The VH may comprise the amino acid sequence of SEQ ID NO: 1036. In some embodiments, the anti-CD3 antibody, or antigen-binding fragment thereof, comprises the VL and VH of antibody ABL-CD3-9A. In some embodiments, the anti-CD3 antibody, or antigen-binding fragment thereof, comprises a VL comprising the amino acid sequence of SEQ ID NO: 809 and a VH comprising the amino acid sequence of SEQ ID NO: 1036. In some embodiments, the anti-CD3 antibody, or antigen-binding fragment thereof, comprises ABL-CD3-9_scFv_VH-VL. In some embodiments, the anti-CD3 antibody, or antigen-binding fragment thereof, comprises, from N-terminus to C-terminus, a VH comprising the amino acid sequence of SEQ ID NO: 923; a linker comprising the amino acid sequence of (Gly-Gly-Gly-Gly-Ser)n (SEQ ID NO: 1146), wherein n is 4; and a VL comprising the amino acid sequence of SEQ ID NO: 696. In some embodiments, the anti-CD3 antibody, or antigen-binding fragment thereof, comprises ABL-CD3-9_scFv_VL-VH. In some embodiments, the anti-CD3 antibody, or antigen-binding fragment thereof, comprises, from N-terminus to C-terminus, a VL comprising the amino acid sequence of SEQ ID NO: 696; a linker comprising the amino acid sequence of (Gly-Gly-Gly-Gly-Ser)n (SEQ ID NO: 1146), wherein n is 4; and a VH comprising the amino acid sequence of SEQ ID NO: 923. In some embodiments, the anti-CD3 antibody, or antigen-binding fragment thereof, comprises ABL-CD3-9A_scFv_VH-VL. In some embodiments, the anti-CD3 antibody, or antigen-binding fragment thereof, comprises, from N-terminus to C-terminus, a VH comprising the amino acid sequence of SEQ ID NO: 1036; a linker comprising the amino acid sequence of (Gly-Gly-Gly-Gly-Ser)n (SEQ ID NO: 1146), wherein n is 4; and a VL comprising the amino acid sequence of SEQ ID NO: 809. In some embodiments, the anti-CD3 antibody, or antigen-binding fragment thereof, comprises ABL-CD3-9A_scFv_VL-VH. In some embodiments, the anti-CD3 antibody, or antigen-binding fragment thereof, comprises, from N-terminus to C-terminus, a VL comprising the amino acid sequence of SEQ ID NO: 809; a linker comprising the amino acid sequence of (Gly-Gly-Gly-Gly-Ser)n (SEQ ID NO: 1146), wherein n is 4; and a VH comprising the amino acid sequence of SEQ ID NO: 1036.

In some embodiments, the anti-CD3 antibody, or antigen-binding fragment thereof, comprises the six CDRs of antibody ABL-CD3-10 and ABL-CD3-10A. The anti-CD3 antibody, or antigen-binding fragment thereof, may comprise an LCDR1, an LCDR2, and an LCDR3 comprising the amino acid sequences of SEQ ID NOs: 10, 123, and 236, respectively; and an HCDR1, an HCDR2, and an HCDR3 comprising the amino acid sequences of SEQ ID NOs: 354, 467, and 583, respectively. In some embodiments, the anti-CD3 antibody, or antigen-binding fragment thereof, comprises the VL of antibody ABL-CD3-10. The VL may comprise the amino acid sequence of SEQ ID NO: 697. In some embodiments, the anti-CD3 antibody, or antigen-binding fragment thereof, comprises the VH of antibody ABL-CD3-10. The VH may comprise the amino acid sequence of SEQ ID NO: 924. In some embodiments, the anti-CD3 antibody, or antigen-binding fragment thereof, comprises the VL and VH of antibody ABL-CD3-10. In some embodiments, the anti-CD3 antibody, or antigen-binding fragment thereof, comprises a VL comprising the amino acid sequence of SEQ ID NO: 697 and a VH comprising the amino acid sequence of SEQ ID NO: 924. In some embodiments, the anti-CD3 antibody, or antigen-binding fragment thereof, comprises the VL of antibody ABL-CD3-10A. The VL may comprise the amino acid sequence of SEQ ID NO: 810. In some embodiments, the anti-CD3 antibody, or antigen-binding fragment thereof, comprises the VH of antibody ABL-CD3-10A. The VH may comprise the amino acid sequence of SEQ ID NO: 1037. In some embodiments, the anti-CD3 antibody, or antigen-binding fragment thereof, comprises the VL and VH of antibody ABL-CD3-10A. In some embodiments, the anti-CD3 antibody, or antigen-binding fragment thereof, comprises a VL comprising the amino acid sequence of SEQ ID NO: 810 and a VH comprising the amino acid sequence of SEQ ID NO: 1037. In some embodiments, the anti-CD3 antibody, or antigen-binding fragment thereof, comprises ABL-CD3-10_scFv_VH-VL. In some embodiments, the anti-CD3 antibody, or antigen-binding fragment thereof, comprises, from N-terminus to C-terminus, a VH comprising the amino acid sequence of SEQ ID NO: 924; a linker comprising the amino acid sequence of (Gly-Gly-Gly-Gly-Ser)n (SEQ ID NO: 1146), wherein n is 4; and a VL comprising the amino acid sequence of SEQ ID NO: 697. In some embodiments, the anti-CD3 antibody, or antigen-binding fragment thereof, comprises ABL-CD3-10_scFv_VL-VH. In some embodiments, the anti-CD3 antibody, or antigen-binding fragment thereof, comprises, from N-terminus to C-terminus, a VL comprising the amino acid sequence of SEQ ID NO: 697; a linker comprising the amino acid sequence of (Gly-Gly-Gly-Gly-Ser)n (SEQ ID NO: 1146), wherein n is 4; and a VH comprising the amino acid sequence of SEQ ID NO: 924. In some embodiments, the anti-CD3 antibody, or antigen-binding fragment thereof, comprises ABL-CD3-10A_scFv_VH-VL. In some embodiments, the anti-CD3 antibody, or antigen-binding fragment thereof, comprises, from N-terminus to C-terminus, a VH comprising the amino acid sequence of SEQ ID NO: 1037; a linker comprising the amino acid sequence of (Gly-Gly-Gly-Gly-Ser)n (SEQ ID NO: 1146), wherein n is 4; and a VL comprising the amino acid sequence of SEQ ID NO: 810. In some embodiments, the anti-CD3 antibody, or antigen-binding fragment thereof, comprises ABL-CD3-10A_scFv_VL-VH. In some embodiments, the anti-CD3 antibody, or antigen-binding fragment thereof, comprises, from N-terminus to C-terminus, a VL comprising the amino acid sequence of SEQ ID NO: 810; a linker comprising the amino acid sequence of (Gly-Gly-Gly-Gly-Ser)n (SEQ ID NO: 1146), wherein n is 4; and a VH comprising the amino acid sequence of SEQ ID NO: 1037.

In some embodiments, the anti-CD3 antibody, or antigen-binding fragment thereof, comprises the six CDRs of antibody ABL-CD3-11 and ABL-CD3-11A. The anti-CD3 antibody, or antigen-binding fragment thereof, may comprise an LCDR1, an LCDR2, and an LCDR3 comprising the amino acid sequences of SEQ ID NOs: 11, 124, and 237, respectively; and an HCDR1, an HCDR2, and an HCDR3 comprising the amino acid sequences of SEQ ID NOs: 355, 468, and 584, respectively. In some embodiments, the anti-CD3 antibody, or antigen-binding fragment thereof, comprises the VL of antibody ABL-CD3-11. The VL may comprise the amino acid sequence of SEQ ID NO: 698. In some embodiments, the anti-CD3 antibody, or antigen-binding fragment thereof, comprises the VH of antibody ABL-CD3-11. The VH may comprise the amino acid sequence of SEQ ID NO: 925. In some embodiments, the anti-CD3 antibody, or antigen-binding fragment thereof, comprises the VL and VH of antibody ABL-CD3-11. In some embodiments, the anti-CD3 antibody, or antigen-binding fragment thereof, comprises a VL comprising the amino acid sequence of SEQ ID NO: 698 and a VH comprising the amino acid sequence of SEQ ID NO: 925. In some embodiments, the anti-CD3 antibody, or antigen-binding fragment thereof, comprises the VL of antibody ABL-CD3-11A. The VL may comprise the amino acid sequence of SEQ ID NO: 811. In some embodiments, the anti-CD3 antibody, or antigen-binding fragment thereof, comprises the VH of antibody ABL-CD3-11A. The VH may comprise the amino acid sequence of SEQ ID NO: 1038. In some embodiments, the anti-CD3 antibody, or antigen-binding fragment thereof, comprises the VL and VH of antibody ABL-CD3-11A. In some embodiments, the anti-CD3 antibody, or antigen-binding fragment thereof, comprises a VL comprising the amino acid sequence of SEQ ID NO: 811 and a VH comprising the amino acid sequence of SEQ ID NO: 1038. In some embodiments, the anti-CD3 antibody, or antigen-binding fragment thereof, comprises ABL-CD3-11_scFv_VH-VL. In some embodiments, the anti-CD3 antibody, or antigen-binding fragment thereof, comprises, from N-terminus to C-terminus, a VH comprising the amino acid sequence of SEQ ID NO: 925; a linker comprising the amino acid sequence of (Gly-Gly-Gly-Gly-Ser)n (SEQ ID NO: 1146), wherein n is 4; and a VL comprising the amino acid sequence of SEQ ID NO: 698. In some embodiments, the anti-CD3 antibody, or antigen-binding fragment thereof, comprises ABL-CD3-11_scFv_VL-VH. In some embodiments, the anti-CD3 antibody, or antigen-binding fragment thereof, comprises, from N-terminus to C-terminus, a VL comprising the amino acid sequence of SEQ ID NO: 698; a linker comprising the amino acid sequence of (Gly-Gly-Gly-Gly-Ser)n (SEQ ID NO: 1146), wherein n is 4; and a VH comprising the amino acid sequence of SEQ ID NO: 925. In some embodiments, the anti-CD3 antibody, or antigen-binding fragment thereof, comprises ABL-CD3-11A_scFv_VH-VL. In some embodiments, the anti-CD3 antibody, or antigen-binding fragment thereof, comprises, from N-terminus to C-terminus, a VH comprising the amino acid sequence of SEQ ID NO: 1038; a linker comprising the amino acid sequence of (Gly-Gly-Gly-Gly-Ser)n (SEQ ID NO: 1146), wherein n is 4; and a VL comprising the amino acid sequence of SEQ ID NO: 811. In some embodiments, the anti-CD3 antibody, or antigen-binding fragment thereof, comprises ABL-CD3-11A_scFv_VL-VH. In some embodiments, the anti-CD3 antibody, or antigen-binding fragment thereof, comprises, from N-terminus to C-terminus, a VL comprising the amino acid sequence of SEQ ID NO: 811; a linker comprising the amino acid sequence of (Gly-Gly-Gly-Gly-Ser)n (SEQ ID NO: 1146), wherein n is 4; and a VH comprising the amino acid sequence of SEQ ID NO: 1038.

In some embodiments, the anti-CD3 antibody, or antigen-binding fragment thereof, comprises the six CDRs of antibody ABL-CD3-12 and ABL-CD3-12A. The anti-CD3 antibody, or antigen-binding fragment thereof, may comprise an LCDR1, an LCDR2, and an LCDR3 comprising the amino acid sequences of SEQ ID NOs: 12, 125, and 238, respectively; and an HCDR1, an HCDR2, and an HCDR3 comprising the amino acid sequences of SEQ ID NOs: 356, 469, and 585, respectively. In some embodiments, the anti-CD3 antibody, or antigen-binding fragment thereof, comprises the VL of antibody ABL-CD3-12. The VL may comprise the amino acid sequence of SEQ ID NO: 699. In some embodiments, the anti-CD3 antibody, or antigen-binding fragment thereof, comprises the VH of antibody ABL-CD3-12. The VH may comprise the amino acid sequence of SEQ ID NO: 926. In some embodiments, the anti-CD3 antibody, or antigen-binding fragment thereof, comprises the VL and VH of antibody ABL-CD3-12. In some embodiments, the anti-CD3 antibody, or antigen-binding fragment thereof, comprises a VL comprising the amino acid sequence of SEQ ID NO: 699 and a VH comprising the amino acid sequence of SEQ ID NO: 926. In some embodiments, the anti-CD3 antibody, or antigen-binding fragment thereof, comprises the VL of antibody ABL-CD3-12A. The VL may comprise the amino acid sequence of SEQ ID NO: 812. In some embodiments, the anti-CD3 antibody, or antigen-binding fragment thereof, comprises the VH of antibody ABL-CD3-12A. The VH may comprise the amino acid sequence of SEQ ID NO: 1039. In some embodiments, the anti-CD3 antibody, or antigen-binding fragment thereof, comprises the VL and VH of antibody ABL-CD3-12A. In some embodiments, the anti-CD3 antibody, or antigen-binding fragment thereof, comprises a VL comprising the amino acid sequence of SEQ ID NO: 812 and a VH comprising the amino acid sequence of SEQ ID NO: 1039. In some embodiments, the anti-CD3 antibody, or antigen-binding fragment thereof, comprises ABL-CD3-12_scFv_VH-VL. In some embodiments, the anti-CD3 antibody, or antigen-binding fragment thereof, comprises, from N-terminus to C-terminus, a VH comprising the amino acid sequence of SEQ ID NO: 926; a linker comprising the amino acid sequence of (Gly-Gly-Gly-Gly-Ser)n (SEQ ID NO: 1146), wherein n is 4; and a VL comprising the amino acid sequence of SEQ ID NO: 699. In some embodiments, the anti-CD3 antibody, or antigen-binding fragment thereof, comprises ABL-CD3-12_scFv_VL-VH. In some embodiments, the anti-CD3 antibody, or antigen-binding fragment thereof, comprises, from N-terminus to C-terminus, a VL comprising the amino acid sequence of SEQ ID NO: 699; a linker comprising the amino acid sequence of (Gly-Gly-Gly-Gly-Ser)n (SEQ ID NO: 1146), wherein n is 4; and a VH comprising the amino acid sequence of SEQ ID NO: 926. In some embodiments, the anti-CD3 antibody, or antigen-binding fragment thereof, comprises ABL-CD3-12A_scFv_VH-VL. In some embodiments, the anti-CD3 antibody, or antigen-binding fragment thereof, comprises, from N-terminus to C-terminus, a VH comprising the amino acid sequence of SEQ ID NO: 1039; a linker comprising the amino acid sequence of (Gly-Gly-Gly-Gly-Ser)n (SEQ ID NO: 1146), wherein n is 4; and a VL comprising the amino acid sequence of SEQ ID NO: 812. In some embodiments, the anti-CD3 antibody, or antigen-binding fragment thereof, comprises ABL-CD3-12A_scFv_VL-VH. In some embodiments, the anti-CD3 antibody, or antigen-binding fragment thereof, comprises, from N-terminus to C-terminus, a VL comprising the amino acid sequence of SEQ ID NO: 812; a linker comprising the amino acid sequence of (Gly-Gly-Gly-Gly-Ser)n (SEQ ID NO: 1146), wherein n is 4; and a VH comprising the amino acid sequence of SEQ ID NO: 1039.

In some embodiments, the anti-CD3 antibody, or antigen-binding fragment thereof, comprises the six CDRs of antibody ABL-CD3-13 and ABL-CD3-13A. The anti-CD3 antibody, or antigen-binding fragment thereof, may comprise an LCDR1, an LCDR2, and an LCDR3 comprising the amino acid sequences of SEQ ID NOs: 13, 126, and 239, respectively; and an HCDR1, an HCDR2, and an HCDR3 comprising the amino acid sequences of SEQ ID NOs: 357, 470, and 586, respectively. In some embodiments, the anti-CD3 antibody, or antigen-binding fragment thereof, comprises the VL of antibody ABL-CD3-13. The VL may comprise the amino acid sequence of SEQ ID NO: 700. In some embodiments, the anti-CD3 antibody, or antigen-binding fragment thereof, comprises the VH of antibody ABL-CD3-13. The VH may comprise the amino acid sequence of SEQ ID NO: 927. In some embodiments, the anti-CD3 antibody, or antigen-binding fragment thereof, comprises the VL and VH of antibody ABL-CD3-13. In some embodiments, the anti-CD3 antibody, or antigen-binding fragment thereof, comprises a VL comprising the amino acid sequence of SEQ ID NO: 700 and a VH comprising the amino acid sequence of SEQ ID NO: 927. In some embodiments, the anti-CD3 antibody, or antigen-binding fragment thereof, comprises the VL of antibody ABL-CD3-13A. The VL may comprise the amino acid sequence of SEQ ID NO: 813. In some embodiments, the anti-CD3 antibody, or antigen-binding fragment thereof, comprises the VH of antibody ABL-CD3-13A. The VH may comprise the amino acid sequence of SEQ ID NO: 1040. In some embodiments, the anti-CD3 antibody, or antigen-binding fragment thereof, comprises the VL and VH of antibody ABL-CD3-13A. In some embodiments, the anti-CD3 antibody, or antigen-binding fragment thereof, comprises a VL comprising the amino acid sequence of SEQ ID NO: 813 and a VH comprising the amino acid sequence of SEQ ID NO: 1040. In some embodiments, the anti-CD3 antibody, or antigen-binding fragment thereof, comprises ABL-CD3-13_scFv_VH-VL. In some embodiments, the anti-CD3 antibody, or antigen-binding fragment thereof, comprises, from N-terminus to C-terminus, a VH comprising the amino acid sequence of SEQ ID NO: 927; a linker comprising the amino acid sequence of (Gly-Gly-Gly-Gly-Ser)n (SEQ ID NO: 1146), wherein n is 4; and a VL comprising the amino acid sequence of SEQ ID NO: 700. In some embodiments, the anti-CD3 antibody, or antigen-binding fragment thereof, comprises ABL-CD3-13_scFv_VL-VH. In some embodiments, the anti-CD3 antibody, or antigen-binding fragment thereof, comprises, from N-terminus to C-terminus, a VL comprising the amino acid sequence of SEQ ID NO: 700; a linker comprising the amino acid sequence of (Gly-Gly-Gly-Gly-Ser)n (SEQ ID NO: 1146), wherein n is 4; and a VH comprising the amino acid sequence of SEQ ID NO: 927. In some embodiments, the anti-CD3 antibody, or antigen-binding fragment thereof, comprises ABL-CD3-13A_scFv_VH-VL. In some embodiments, the anti-CD3 antibody, or antigen-binding fragment thereof, comprises, from N-terminus to C-terminus, a VH comprising the amino acid sequence of SEQ ID NO: 1040; a linker comprising the amino acid sequence of (Gly-Gly-Gly-Gly-Ser)n (SEQ ID NO: 1146), wherein n is 4; and a VL comprising the amino acid sequence of SEQ ID NO: 813. In some embodiments, the anti-CD3 antibody, or antigen-binding fragment thereof, comprises ABL-CD3-13A_scFv_VL-VH. In some embodiments, the anti-CD3 antibody, or antigen-binding fragment thereof, comprises, from N-terminus to C-terminus, a VL comprising the amino acid sequence of SEQ ID NO: 813; a linker comprising the amino acid sequence of (Gly-Gly-Gly-Gly-Ser)n (SEQ ID NO: 1146), wherein n is 4; and a VH comprising the amino acid sequence of SEQ ID NO: 1040.

In some embodiments, the anti-CD3 antibody, or antigen-binding fragment thereof, comprises the six CDRs of antibody ABL-CD3-14 and ABL-CD3-14A. The anti-CD3 antibody, or antigen-binding fragment thereof, may comprise an LCDR1, an LCDR2, and an LCDR3 comprising the amino acid sequences of SEQ ID NOs: 14, 127, and 240, respectively; and an HCDR1, an HCDR2, and an HCDR3 comprising the amino acid sequences of SEQ ID NOs: 358, 471, and 587, respectively. In some embodiments, the anti-CD3 antibody, or antigen-binding fragment thereof, comprises the VL of antibody ABL-CD3-14. The VL may comprise the amino acid sequence of SEQ ID NO: 701. In some embodiments, the anti-CD3 antibody, or antigen-binding fragment thereof, comprises the VH of antibody ABL-CD3-14. The VH may comprise the amino acid sequence of SEQ ID NO: 928. In some embodiments, the anti-CD3 antibody, or antigen-binding fragment thereof, comprises the VL and VH of antibody ABL-CD3-14. In some embodiments, the anti-CD3 antibody, or antigen-binding fragment thereof, comprises a VL comprising the amino acid sequence of SEQ ID NO: 701 and a VH comprising the amino acid sequence of SEQ ID NO: 928. In some embodiments, the anti-CD3 antibody, or antigen-binding fragment thereof, comprises the VL of antibody ABL-CD3-14A. The VL may comprise the amino acid sequence of SEQ ID NO: 814. In some embodiments, the anti-CD3 antibody, or antigen-binding fragment thereof, comprises the VH of antibody ABL-CD3-14A. The VH may comprise the amino acid sequence of SEQ ID NO: 1041. In some embodiments, the anti-CD3 antibody, or antigen-binding fragment thereof, comprises the VL and VH of antibody ABL-CD3-14A. In some embodiments, the anti-CD3 antibody, or antigen-binding fragment thereof, comprises a VL comprising the amino acid sequence of SEQ ID NO: 814 and a VH comprising the amino acid sequence of SEQ ID NO: 1041. In some embodiments, the anti-CD3 antibody, or antigen-binding fragment thereof, comprises ABL-CD3-14_scFv_VH-VL. In some embodiments, the anti-CD3 antibody, or antigen-binding fragment thereof, comprises, from N-terminus to C-terminus, a VH comprising the amino acid sequence of SEQ ID NO: 928; a linker comprising the amino acid sequence of (Gly-Gly-Gly-Gly-Ser)n (SEQ ID NO: 1146), wherein n is 4; and a VL comprising the amino acid sequence of SEQ ID NO: 701. In some embodiments, the anti-CD3 antibody, or antigen-binding fragment thereof, comprises ABL-CD3-14_scFv_VL-VH. In some embodiments, the anti-CD3 antibody, or antigen-binding fragment thereof, comprises, from N-terminus to C-terminus, a VL comprising the amino acid sequence of SEQ ID NO: 701; a linker comprising the amino acid sequence of (Gly-Gly-Gly-Gly-Ser)n (SEQ ID NO: 1146), wherein n is 4; and a VH comprising the amino acid sequence of SEQ ID NO: 928. In some embodiments, the anti-CD3 antibody, or antigen-binding fragment thereof, comprises ABL-CD3-14A_scFv_VH-VL. In some embodiments, the anti-CD3 antibody, or antigen-binding fragment thereof, comprises, from N-terminus to C-terminus, a VH comprising the amino acid sequence of SEQ ID NO: 1041; a linker comprising the amino acid sequence of (Gly-Gly-Gly-Gly-Ser)n (SEQ ID NO: 1146), wherein n is 4; and a VL comprising the amino acid sequence of SEQ ID NO: 814. In some embodiments, the anti-CD3 antibody, or antigen-binding fragment thereof, comprises ABL-CD3-14A_scFv_VL-VH. In some embodiments, the anti-CD3 antibody, or antigen-binding fragment thereof, comprises, from N-terminus to C-terminus, a VL comprising the amino acid sequence of SEQ ID NO: 814; a linker comprising the amino acid sequence of (Gly-Gly-Gly-Gly-Ser)n (SEQ ID NO: 1146), wherein n is 4; and a VH comprising the amino acid sequence of SEQ ID NO: 1041.

In some embodiments, the anti-CD3 antibody, or antigen-binding fragment thereof, comprises the six CDRs of antibody ABL-CD3-15 and ABL-CD3-15A. The anti-CD3 antibody, or antigen-binding fragment thereof, may comprise an LCDR1, an LCDR2, and an LCDR3 comprising the amino acid sequences of SEQ ID NOs: 15, 128, and 241, respectively; and an HCDR1, an HCDR2, and an HCDR3 comprising the amino acid sequences of SEQ ID NOs: 359, 472, and 588, respectively. In some embodiments, the anti-CD3 antibody, or antigen-binding fragment thereof, comprises the VL of antibody ABL-CD3-15. The VL may comprise the amino acid sequence of SEQ ID NO: 702. In some embodiments, the anti-CD3 antibody, or antigen-binding fragment thereof, comprises the VH of antibody ABL-CD3-15. The VH may comprise the amino acid sequence of SEQ ID NO: 929. In some embodiments, the anti-CD3 antibody, or antigen-binding fragment thereof, comprises the VL and VH of antibody ABL-CD3-15. In some embodiments, the anti-CD3 antibody, or antigen-binding fragment thereof, comprises a VL comprising the amino acid sequence of SEQ ID NO: 702 and a VH comprising the amino acid sequence of SEQ ID NO: 929. In some embodiments, the anti-CD3 antibody, or antigen-binding fragment thereof, comprises the VL of antibody ABL-CD3-15A. The VL may comprise the amino acid sequence of SEQ ID NO: 815. In some embodiments, the anti-CD3 antibody, or antigen-binding fragment thereof, comprises the VH of antibody ABL-CD3-15A. The VH may comprise the amino acid sequence of SEQ ID NO: 1042. In some embodiments, the anti-CD3 antibody, or antigen-binding fragment thereof, comprises the VL and VH of antibody ABL-CD3-15A. In some embodiments, the anti-CD3 antibody, or antigen-binding fragment thereof, comprises a VL comprising the amino acid sequence of SEQ ID NO: 815 and a VH comprising the amino acid sequence of SEQ ID NO: 1042. In some embodiments, the anti-CD3 antibody, or antigen-binding fragment thereof, comprises ABL-CD3-15_scFv_VH-VL. In some embodiments, the anti-CD3 antibody, or antigen-binding fragment thereof, comprises, from N-terminus to C-terminus, a VH comprising the amino acid sequence of SEQ ID NO: 929; a linker comprising the amino acid sequence of (Gly-Gly-Gly-Gly-Ser)n (SEQ ID NO: 1146), wherein n is 4; and a VL comprising the amino acid sequence of SEQ ID NO: 702. In some embodiments, the anti-CD3 antibody, or antigen-binding fragment thereof, comprises ABL-CD3-15_scFv_VL-VH. In some embodiments, the anti-CD3 antibody, or antigen-binding fragment thereof, comprises, from N-terminus to C-terminus, a VL comprising the amino acid sequence of SEQ ID NO: 702; a linker comprising the amino acid sequence of (Gly-Gly-Gly-Gly-Ser)n (SEQ ID NO: 1146), wherein n is 4; and a VH comprising the amino acid sequence of SEQ ID NO: 929. In some embodiments, the anti-CD3 antibody, or antigen-binding fragment thereof, comprises ABL-CD3-15A_scFv_VH-VL. In some embodiments, the anti-CD3 antibody, or antigen-binding fragment thereof, comprises, from N-terminus to C-terminus, a VH comprising the amino acid sequence of SEQ ID NO: 1042; a linker comprising the amino acid sequence of (Gly-Gly-Gly-Gly-Ser)n (SEQ ID NO: 1146), wherein n is 4; and a VL comprising the amino acid sequence of SEQ ID NO: 815. In some embodiments, the anti-CD3 antibody, or antigen-binding fragment thereof, comprises ABL-CD3-15A_scFv_VL-VH. In some embodiments, the anti-CD3 antibody, or antigen-binding fragment thereof, comprises, from N-terminus to C-terminus, a VL comprising the amino acid sequence of SEQ ID NO: 815; a linker comprising the amino acid sequence of (Gly-Gly-Gly-Gly-Ser)n (SEQ ID NO: 1146), wherein n is 4; and a VH comprising the amino acid sequence of SEQ ID NO: 1042.

In some embodiments, the anti-CD3 antibody, or antigen-binding fragment thereof, comprises the six CDRs of antibody ABL-CD3-16 and ABL-CD3-16A. The anti-CD3 antibody, or antigen-binding fragment thereof, may comprise an LCDR1, an LCDR2, and an LCDR3 comprising the amino acid sequences of SEQ ID NOs: 16, 129, and 242, respectively; and an HCDR1, an HCDR2, and an HCDR3 comprising the amino acid sequences of SEQ ID NOs: 360, 473, and 589, respectively. In some embodiments, the anti-CD3 antibody, or antigen-binding fragment thereof, comprises the VL of antibody ABL-CD3-16. The VL may comprise the amino acid sequence of SEQ ID NO: 703. In some embodiments, the anti-CD3 antibody, or antigen-binding fragment thereof, comprises the VH of antibody ABL-CD3-16. The VH may comprise the amino acid sequence of SEQ ID NO: 930. In some embodiments, the anti-CD3 antibody, or antigen-binding fragment thereof, comprises the VL and VH of antibody ABL-CD3-16. In some embodiments, the anti-CD3 antibody, or antigen-binding fragment thereof, comprises a VL comprising the amino acid sequence of SEQ ID NO: 703 and a VH comprising the amino acid sequence of SEQ ID NO: 930. In some embodiments, the anti-CD3 antibody, or antigen-binding fragment thereof, comprises the VL of antibody ABL-CD3-16A. The VL may comprise the amino acid sequence of SEQ ID NO: 816. In some embodiments, the anti-CD3 antibody, or antigen-binding fragment thereof, comprises the VH of antibody ABL-CD3-16A. The VH may comprise the amino acid sequence of SEQ ID NO: 1043. In some embodiments, the anti-CD3 antibody, or antigen-binding fragment thereof, comprises the VL and VH of antibody ABL-CD3-16A. In some embodiments, the anti-CD3 antibody, or antigen-binding fragment thereof, comprises a VL comprising the amino acid sequence of SEQ ID NO: 816 and a VH comprising the amino acid sequence of SEQ ID NO: 1043. In some embodiments, the anti-CD3 antibody, or antigen-binding fragment thereof, comprises ABL-CD3-16_scFv_VH-VL. In some embodiments, the anti-CD3 antibody, or antigen-binding fragment thereof, comprises, from N-terminus to C-terminus, a VH comprising the amino acid sequence of SEQ ID NO: 930; a linker comprising the amino acid sequence of (Gly-Gly-Gly-Gly-Ser)n (SEQ ID NO: 1146), wherein n is 4; and a VL comprising the amino acid sequence of SEQ ID NO: 703. In some embodiments, the anti-CD3 antibody, or antigen-binding fragment thereof, comprises ABL-CD3-16_scFv_VL-VH. In some embodiments, the anti-CD3 antibody, or antigen-binding fragment thereof, comprises, from N-terminus to C-terminus, a VL comprising the amino acid sequence of SEQ ID NO: 703; a linker comprising the amino acid sequence of (Gly-Gly-Gly-Gly-Ser)n (SEQ ID NO: 1146), wherein n is 4; and a VH comprising the amino acid sequence of SEQ ID NO: 930. In some embodiments, the anti-CD3 antibody, or antigen-binding fragment thereof, comprises ABL-CD3-16A_scFv_VH-VL. In some embodiments, the anti-CD3 antibody, or antigen-binding fragment thereof, comprises, from N-terminus to C-terminus, a VH comprising the amino acid sequence of SEQ ID NO: 1043; a linker comprising the amino acid sequence of (Gly-Gly-Gly-Gly-Ser)n (SEQ ID NO: 1146), wherein n is 4; and a VL comprising the amino acid sequence of SEQ ID NO: 816. In some embodiments, the anti-CD3 antibody, or antigen-binding fragment thereof, comprises ABL-CD3-16A_scFv_VL-VH. In some embodiments, the anti-CD3 antibody, or antigen-binding fragment thereof, comprises, from N-terminus to C-terminus, a VL comprising the amino acid sequence of SEQ ID NO: 816; a linker comprising the amino acid sequence of (Gly-Gly-Gly-Gly-Ser)n (SEQ ID NO: 1146), wherein n is 4; and a VH comprising the amino acid sequence of SEQ ID NO: 1043.

In some embodiments, the anti-CD3 antibody, or antigen-binding fragment thereof, comprises the six CDRs of antibody ABL-CD3-17 and ABL-CD3-17A. The anti-CD3 antibody, or antigen-binding fragment thereof, may comprise an LCDR1, an LCDR2, and an LCDR3 comprising the amino acid sequences of SEQ ID NOs: 17, 130, and 243, respectively; and an HCDR1, an HCDR2, and an HCDR3 comprising the amino acid sequences of SEQ ID NOs: 361, 474, and 590, respectively. In some embodiments, the anti-CD3 antibody, or antigen-binding fragment thereof, comprises the VL of antibody ABL-CD3-17. The VL may comprise the amino acid sequence of SEQ ID NO: 704. In some embodiments, the anti-CD3 antibody, or antigen-binding fragment thereof, comprises the VH of antibody ABL-CD3-17. The VH may comprise the amino acid sequence of SEQ ID NO: 931. In some embodiments, the anti-CD3 antibody, or antigen-binding fragment thereof, comprises the VL and VH of antibody ABL-CD3-17. In some embodiments, the anti-CD3 antibody, or antigen-binding fragment thereof, comprises a VL comprising the amino acid sequence of SEQ ID NO: 704 and a VH comprising the amino acid sequence of SEQ ID NO: 931. In some embodiments, the anti-CD3 antibody, or antigen-binding fragment thereof, comprises the VL of antibody ABL-CD3-17A. The VL may comprise the amino acid sequence of SEQ ID NO: 817. In some embodiments, the anti-CD3 antibody, or antigen-binding fragment thereof, comprises the VH of antibody ABL-CD3-17A. The VH may comprise the amino acid sequence of SEQ ID NO: 1044. In some embodiments, the anti-CD3 antibody, or antigen-binding fragment thereof, comprises the VL and VH of antibody ABL-CD3-17A. In some embodiments, the anti-CD3 antibody, or antigen-binding fragment thereof, comprises a VL comprising the amino acid sequence of SEQ ID NO: 817 and a VH comprising the amino acid sequence of SEQ ID NO: 1044. In some embodiments, the anti-CD3 antibody, or antigen-binding fragment thereof, comprises ABL-CD3-17_scFv_VH-VL. In some embodiments, the anti-CD3 antibody, or antigen-binding fragment thereof, comprises, from N-terminus to C-terminus, a VH comprising the amino acid sequence of SEQ ID NO: 931; a linker comprising the amino acid sequence of (Gly-Gly-Gly-Gly-Ser)n (SEQ ID NO: 1146), wherein n is 4; and a VL comprising the amino acid sequence of SEQ ID NO: 704. In some embodiments, the anti-CD3 antibody, or antigen-binding fragment thereof, comprises ABL-CD3-17_scFv_VL-VH. In some embodiments, the anti-CD3 antibody, or antigen-binding fragment thereof, comprises, from N-terminus to C-terminus, a VL comprising the amino acid sequence of SEQ ID NO: 704; a linker comprising the amino acid sequence of (Gly-Gly-Gly-Gly-Ser)n (SEQ ID NO: 1146), wherein n is 4; and a VH comprising the amino acid sequence of SEQ ID NO: 931. In some embodiments, the anti-CD3 antibody, or antigen-binding fragment thereof, comprises ABL-CD3-17A_scFv_VH-VL. In some embodiments, the anti-CD3 antibody, or antigen-binding fragment thereof, comprises, from N-terminus to C-terminus, a VH comprising the amino acid sequence of SEQ ID NO: 1044; a linker comprising the amino acid sequence of (Gly-Gly-Gly-Gly-Ser)n (SEQ ID NO: 1146), wherein n is 4; and a VL comprising the amino acid sequence of SEQ ID NO: 817. In some embodiments, the anti-CD3 antibody, or antigen-binding fragment thereof, comprises ABL-CD3-17A_scFv_VL-VH. In some embodiments, the anti-CD3 antibody, or antigen-binding fragment thereof, comprises, from N-terminus to C-terminus, a VL comprising the amino acid sequence of SEQ ID NO: 817; a linker comprising the amino acid sequence of (Gly-Gly-Gly-Gly-Ser)n (SEQ ID NO: 1146), wherein n is 4; and a VH comprising the amino acid sequence of SEQ ID NO: 1044.

In some embodiments, the anti-CD3 antibody, or antigen-binding fragment thereof, comprises the six CDRs of antibody ABL-CD3-18 and ABL-CD3-18A. The anti-CD3 antibody, or antigen-binding fragment thereof, may comprise an LCDR1, an LCDR2, and an LCDR3 comprising the amino acid sequences of SEQ ID NOs: 18, 131, and 244, respectively; and an HCDR1, an HCDR2, and an HCDR3 comprising the amino acid sequences of SEQ ID NOs: 362, 475, and 591, respectively. In some embodiments, the anti-CD3 antibody, or antigen-binding fragment thereof, comprises the VL of antibody ABL-CD3-18. The VL may comprise the amino acid sequence of SEQ ID NO: 705. In some embodiments, the anti-CD3 antibody, or antigen-binding fragment thereof, comprises the VH of antibody ABL-CD3-18. The VH may comprise the amino acid sequence of SEQ ID NO: 932. In some embodiments, the anti-CD3 antibody, or antigen-binding fragment thereof, comprises the VL and VH of antibody ABL-CD3-18. In some embodiments, the anti-CD3 antibody, or antigen-binding fragment thereof, comprises a VL comprising the amino acid sequence of SEQ ID NO: 705 and a VH comprising the amino acid sequence of SEQ ID NO: 932. In some embodiments, the anti-CD3 antibody, or antigen-binding fragment thereof, comprises the VL of antibody ABL-CD3-18A. The VL may comprise the amino acid sequence of SEQ ID NO: 818. In some embodiments, the anti-CD3 antibody, or antigen-binding fragment thereof, comprises the VH of antibody ABL-CD3-18A. The VH may comprise the amino acid sequence of SEQ ID NO: 1045. In some embodiments, the anti-CD3 antibody, or antigen-binding fragment thereof, comprises the VL and VH of antibody ABL-CD3-18A. In some embodiments, the anti-CD3 antibody, or antigen-binding fragment thereof, comprises a VL comprising the amino acid sequence of SEQ ID NO: 818 and a VH comprising the amino acid sequence of SEQ ID NO: 1045. In some embodiments, the anti-CD3 antibody, or antigen-binding fragment thereof, comprises ABL-CD3-18_scFv_VH-VL. In some embodiments, the anti-CD3 antibody, or antigen-binding fragment thereof, comprises, from N-terminus to C-terminus, a VH comprising the amino acid sequence of SEQ ID NO: 932; a linker comprising the amino acid sequence of (Gly-Gly-Gly-Gly-Ser)n (SEQ ID NO: 1146), wherein n is 4; and a VL comprising the amino acid sequence of SEQ ID NO: 705. In some embodiments, the anti-CD3 antibody, or antigen-binding fragment thereof, comprises ABL-CD3-18_scFv_VL-VH. In some embodiments, the anti-CD3 antibody, or antigen-binding fragment thereof, comprises, from N-terminus to C-terminus, a VL comprising the amino acid sequence of SEQ ID NO: 705; a linker comprising the amino acid sequence of (Gly-Gly-Gly-Gly-Ser)n (SEQ ID NO: 1146), wherein n is 4; and a VH comprising the amino acid sequence of SEQ ID NO: 932. In some embodiments, the anti-CD3 antibody, or antigen-binding fragment thereof, comprises ABL-CD3-18A_scFv_VH-VL. In some embodiments, the anti-CD3 antibody, or antigen-binding fragment thereof, comprises the amino acid sequence of SEQ ID NO: 1155. In some embodiments, the anti-CD3 antibody, or antigen-binding fragment thereof, comprises ABL-CD3-18A_scFv_VL-VH. In some embodiments, the anti-CD3 antibody, or antigen-binding fragment thereof, comprises the amino acid sequence of SEQ ID NO: 1196.

In some embodiments, the anti-CD3 antibody, or antigen-binding fragment thereof, comprises the six CDRs of antibody ABL-CD3-19 and ABL-CD3-19A. The anti-CD3 antibody, or antigen-binding fragment thereof, may comprise an LCDR1, an LCDR2, and an LCDR3 comprising the amino acid sequences of SEQ ID NOs: 19, 132, and 245, respectively; and an HCDR1, an HCDR2, and an HCDR3 comprising the amino acid sequences of SEQ ID NOs: 363, 476, and 592, respectively. In some embodiments, the anti-CD3 antibody, or antigen-binding fragment thereof, comprises the VL of antibody ABL-CD3-19. The VL may comprise the amino acid sequence of SEQ ID NO: 706. In some embodiments, the anti-CD3 antibody, or antigen-binding fragment thereof, comprises the VH of antibody ABL-CD3-19. The VH may comprise the amino acid sequence of SEQ ID NO: 933. In some embodiments, the anti-CD3 antibody, or antigen-binding fragment thereof, comprises the VL and VH of antibody ABL-CD3-19. In some embodiments, the anti-CD3 antibody, or antigen-binding fragment thereof, comprises a VL comprising the amino acid sequence of SEQ ID NO: 706 and a VH comprising the amino acid sequence of SEQ ID NO: 933. In some embodiments, the anti-CD3 antibody, or antigen-binding fragment thereof, comprises the VL of antibody ABL-CD3-19A. The VL may comprise the amino acid sequence of SEQ ID NO: 819. In some embodiments, the anti-CD3 antibody, or antigen-binding fragment thereof, comprises the VH of antibody ABL-CD3-19A. The VH may comprise the amino acid sequence of SEQ ID NO: 1046. In some embodiments, the anti-CD3 antibody, or antigen-binding fragment thereof, comprises the VL and VH of antibody ABL-CD3-19A. In some embodiments, the anti-CD3 antibody, or antigen-binding fragment thereof, comprises a VL comprising the amino acid sequence of SEQ ID NO: 819 and a VH comprising the amino acid sequence of SEQ ID NO: 1046. In some embodiments, the anti-CD3 antibody, or antigen-binding fragment thereof, comprises ABL-CD3-19_scFv_VH-VL. In some embodiments, the anti-CD3 antibody, or antigen-binding fragment thereof, comprises, from N-terminus to C-terminus, a VH comprising the amino acid sequence of SEQ ID NO: 933; a linker comprising the amino acid sequence of (Gly-Gly-Gly-Gly-Ser)n (SEQ ID NO: 1146), wherein n is 4; and a VL comprising the amino acid sequence of SEQ ID NO: 706. In some embodiments, the anti-CD3 antibody, or antigen-binding fragment thereof, comprises ABL-CD3-19_scFv_VL-VH. In some embodiments, the anti-CD3 antibody, or antigen-binding fragment thereof, comprises, from N-terminus to C-terminus, a VL comprising the amino acid sequence of SEQ ID NO: 706; a linker comprising the amino acid sequence of (Gly-Gly-Gly-Gly-Ser)n (SEQ ID NO: 1146), wherein n is 4; and a VH comprising the amino acid sequence of SEQ ID NO: 933. In some embodiments, the anti-CD3 antibody, or antigen-binding fragment thereof, comprises ABL-CD3-19A_scFv_VH-VL. In some embodiments, the anti-CD3 antibody, or antigen-binding fragment thereof, comprises, from N-terminus to C-terminus, a VH comprising the amino acid sequence of SEQ ID NO: 1046; a linker comprising the amino acid sequence of (Gly-Gly-Gly-Gly-Ser)n (SEQ ID NO: 1146), wherein n is 4; and a VL comprising the amino acid sequence of SEQ ID NO: 819. In some embodiments, the anti-CD3 antibody, or antigen-binding fragment thereof, comprises ABL-CD3-19A_scFv_VL-VH. In some embodiments, the anti-CD3 antibody, or antigen-binding fragment thereof, comprises, from N-terminus to C-terminus, a VL comprising the amino acid sequence of SEQ ID NO: 819; a linker comprising the amino acid sequence of (Gly-Gly-Gly-Gly-Ser)n (SEQ ID NO: 1146), wherein n is 4; and a VH comprising the amino acid sequence of SEQ ID NO: 1046.

In some embodiments, the anti-CD3 antibody, or antigen-binding fragment thereof, comprises the six CDRs of antibody ABL-CD3-20 and ABL-CD3-20A. The anti-CD3 antibody, or antigen-binding fragment thereof, may comprise an LCDR1, an LCDR2, and an LCDR3 comprising the amino acid sequences of SEQ ID NOs: 20, 133, and 246, respectively; and an HCDR1, an HCDR2, and an HCDR3 comprising the amino acid sequences of SEQ ID NOs: 364, 477, and 593, respectively. In some embodiments, the anti-CD3 antibody, or antigen-binding fragment thereof, comprises the VL of antibody ABL-CD3-20. The VL may comprise the amino acid sequence of SEQ ID NO: 707. In some embodiments, the anti-CD3 antibody, or antigen-binding fragment thereof, comprises the VH of antibody ABL-CD3-20. The VH may comprise the amino acid sequence of SEQ ID NO: 934. In some embodiments, the anti-CD3 antibody, or antigen-binding fragment thereof, comprises the VL and VH of antibody ABL-CD3-20. In some embodiments, the anti-CD3 antibody, or antigen-binding fragment thereof, comprises a VL comprising the amino acid sequence of SEQ ID NO: 707 and a VH comprising the amino acid sequence of SEQ ID NO: 934. In some embodiments, the anti-CD3 antibody, or antigen-binding fragment thereof, comprises the VL of antibody ABL-CD3-20A. The VL may comprise the amino acid sequence of SEQ ID NO: 820. In some embodiments, the anti-CD3 antibody, or antigen-binding fragment thereof, comprises the VH of antibody ABL-CD3-20A. The VH may comprise the amino acid sequence of SEQ ID NO: 1047. In some embodiments, the anti-CD3 antibody, or antigen-binding fragment thereof, comprises the VL and VH of antibody ABL-CD3-20A. In some embodiments, the anti-CD3 antibody, or antigen-binding fragment thereof, comprises a VL comprising the amino acid sequence of SEQ ID NO: 820 and a VH comprising the amino acid sequence of SEQ ID NO: 1047. In some embodiments, the anti-CD3 antibody, or antigen-binding fragment thereof, comprises ABL-CD3-20_scFv_VH-VL. In some embodiments, the anti-CD3 antibody, or antigen-binding fragment thereof, comprises, from N-terminus to C-terminus, a VH comprising the amino acid sequence of SEQ ID NO: 934; a linker comprising the amino acid sequence of (Gly-Gly-Gly-Gly-Ser)n (SEQ ID NO: 1146), wherein n is 4; and a VL comprising the amino acid sequence of SEQ ID NO: 707. In some embodiments, the anti-CD3 antibody, or antigen-binding fragment thereof, comprises ABL-CD3-20_scFv_VL-VH. In some embodiments, the anti-CD3 antibody, or antigen-binding fragment thereof, comprises, from N-terminus to C-terminus, a VL comprising the amino acid sequence of SEQ ID NO: 707; a linker comprising the amino acid sequence of (Gly-Gly-Gly-Gly-Ser)n (SEQ ID NO: 1146), wherein n is 4; and a VH comprising the amino acid sequence of SEQ ID NO: 934. In some embodiments, the anti-CD3 antibody, or antigen-binding fragment thereof, comprises ABL-CD3-20A_scFv_VH-VL. In some embodiments, the anti-CD3 antibody, or antigen-binding fragment thereof, comprises the amino acid sequence of SEQ ID NO: 1156. In some embodiments, the anti-CD3 antibody, or antigen-binding fragment thereof, comprises ABL-CD3-20A_scFv_VL-VH. In some embodiments, the anti-CD3 antibody, or antigen-binding fragment thereof, comprises the amino acid sequence of SEQ ID NO: 1197.

In some embodiments, the anti-CD3 antibody, or antigen-binding fragment thereof, comprises the six CDRs of antibody ABL-CD3-21 and ABL-CD3-21A. The anti-CD3 antibody, or antigen-binding fragment thereof, may comprise an LCDR1, an LCDR2, and an LCDR3 comprising the amino acid sequences of SEQ ID NOs: 21, 134, and 247, respectively; and an HCDR1, an HCDR2, and an HCDR3 comprising the amino acid sequences of SEQ ID NOs: 365, 478, and 594, respectively. In some embodiments, the anti-CD3 antibody, or antigen-binding fragment thereof, comprises the VL of antibody ABL-CD3-21. The VL may comprise the amino acid sequence of SEQ ID NO: 708. In some embodiments, the anti-CD3 antibody, or antigen-binding fragment thereof, comprises the VH of antibody ABL-CD3-21. The VH may comprise the amino acid sequence of SEQ ID NO: 935. In some embodiments, the anti-CD3 antibody, or antigen-binding fragment thereof, comprises the VL and VH of antibody ABL-CD3-21. In some embodiments, the anti-CD3 antibody, or antigen-binding fragment thereof, comprises a VL comprising the amino acid sequence of SEQ ID NO: 708 and a VH comprising the amino acid sequence of SEQ ID NO: 935. In some embodiments, the anti-CD3 antibody, or antigen-binding fragment thereof, comprises the VL of antibody ABL-CD3-21A. The VL may comprise the amino acid sequence of SEQ ID NO: 821. In some embodiments, the anti-CD3 antibody, or antigen-binding fragment thereof, comprises the VH of antibody ABL-CD3-21A. The VH may comprise the amino acid sequence of SEQ ID NO: 1048. In some embodiments, the anti-CD3 antibody, or antigen-binding fragment thereof, comprises the VL and VH of antibody ABL-CD3-21A. In some embodiments, the anti-CD3 antibody, or antigen-binding fragment thereof, comprises a VL comprising the amino acid sequence of SEQ ID NO: 821 and a VH comprising the amino acid sequence of SEQ ID NO: 1048. In some embodiments, the anti-CD3 antibody, or antigen-binding fragment thereof, comprises ABL-CD3-21_scFv_VH-VL. In some embodiments, the anti-CD3 antibody, or antigen-binding fragment thereof, comprises, from N-terminus to C-terminus, a VH comprising the amino acid sequence of SEQ ID NO: 935; a linker comprising the amino acid sequence of (Gly-Gly-Gly-Gly-Ser)n (SEQ ID NO: 1146), wherein n is 4; and a VL comprising the amino acid sequence of SEQ ID NO: 708. In some embodiments, the anti-CD3 antibody, or antigen-binding fragment thereof, comprises ABL-CD3-21_scFv_VL-VH. In some embodiments, the anti-CD3 antibody, or antigen-binding fragment thereof, comprises, from N-terminus to C-terminus, a VL comprising the amino acid sequence of SEQ ID NO: 708; a linker comprising the amino acid sequence of (Gly-Gly-Gly-Gly-Ser)n (SEQ ID NO: 1146), wherein n is 4; and a VH comprising the amino acid sequence of SEQ ID NO: 935. In some embodiments, the anti-CD3 antibody, or antigen-binding fragment thereof, comprises ABL-CD3-21A_scFv_VH-VL. In some embodiments, the anti-CD3 antibody, or antigen-binding fragment thereof, comprises the amino acid sequence of SEQ ID NO: 1157. In some embodiments, the anti-CD3 antibody, or antigen-binding fragment thereof, comprises ABL-CD3-21A_scFv_VL-VH. In some embodiments, the anti-CD3 antibody, or antigen-binding fragment thereof, comprises the amino acid sequence of SEQ ID NO: 1198.

In some embodiments, the anti-CD3 antibody, or antigen-binding fragment thereof, comprises the six CDRs of antibody ABL-CD3-22 and ABL-CD3-22A. The anti-CD3 antibody, or antigen-binding fragment thereof, may comprise an LCDR1, an LCDR2, and an LCDR3 comprising the amino acid sequences of SEQ ID NOs: 22, 135, and 248, respectively; and an HCDR1, an HCDR2, and an HCDR3 comprising the amino acid sequences of SEQ ID NOs: 366, 479, and 595, respectively. In some embodiments, the anti-CD3 antibody, or antigen-binding fragment thereof, comprises the VL of antibody ABL-CD3-22. The VL may comprise the amino acid sequence of SEQ ID NO: 709. In some embodiments, the anti-CD3 antibody, or antigen-binding fragment thereof, comprises the VH of antibody ABL-CD3-22. The VH may comprise the amino acid sequence of SEQ ID NO: 936. In some embodiments, the anti-CD3 antibody, or antigen-binding fragment thereof, comprises the VL and VH of antibody ABL-CD3-22. In some embodiments, the anti-CD3 antibody, or antigen-binding fragment thereof, comprises a VL comprising the amino acid sequence of SEQ ID NO: 709 and a VH comprising the amino acid sequence of SEQ ID NO: 936. In some embodiments, the anti-CD3 antibody, or antigen-binding fragment thereof, comprises the VL of antibody ABL-CD3-22A. The VL may comprise the amino acid sequence of SEQ ID NO: 822. In some embodiments, the anti-CD3 antibody, or antigen-binding fragment thereof, comprises the VH of antibody ABL-CD3-22A. The VH may comprise the amino acid sequence of SEQ ID NO: 1049. In some embodiments, the anti-CD3 antibody, or antigen-binding fragment thereof, comprises the VL and VH of antibody ABL-CD3-22A. In some embodiments, the anti-CD3 antibody, or antigen-binding fragment thereof, comprises a VL comprising the amino acid sequence of SEQ ID NO: 822 and a VH comprising the amino acid sequence of SEQ ID NO: 1049. In some embodiments, the anti-CD3 antibody, or antigen-binding fragment thereof, comprises ABL-CD3-22_scFv_VH-VL. In some embodiments, the anti-CD3 antibody, or antigen-binding fragment thereof, comprises, from N-terminus to C-terminus, a VH comprising the amino acid sequence of SEQ ID NO: 936; a linker comprising the amino acid sequence of (Gly-Gly-Gly-Gly-Ser)n (SEQ ID NO: 1146), wherein n is 4; and a VL comprising the amino acid sequence of SEQ ID NO: 709. In some embodiments, the anti-CD3 antibody, or antigen-binding fragment thereof, comprises ABL-CD3-22_scFv_VL-VH. In some embodiments, the anti-CD3 antibody, or antigen-binding fragment thereof, comprises, from N-terminus to C-terminus, a VL comprising the amino acid sequence of SEQ ID NO: 709; a linker comprising the amino acid sequence of (Gly-Gly-Gly-Gly-Ser)n (SEQ ID NO: 1146), wherein n is 4; and a VH comprising the amino acid sequence of SEQ ID NO: 936. In some embodiments, the anti-CD3 antibody, or antigen-binding fragment thereof, comprises ABL-CD3-22A_scFv_VH-VL. In some embodiments, the anti-CD3 antibody, or antigen-binding fragment thereof, comprises the amino acid sequence of SEQ ID NO: 1158. In some embodiments, the anti-CD3 antibody, or antigen-binding fragment thereof, comprises ABL-CD3-22A_scFv_VL-VH. In some embodiments, the anti-CD3 antibody, or antigen-binding fragment thereof, comprises the amino acid sequence of SEQ ID NO: 1199.

In some embodiments, the anti-CD3 antibody, or antigen-binding fragment thereof, comprises the six CDRs of antibody ABL-CD3-23 and ABL-CD3-23A. The anti-CD3 antibody, or antigen-binding fragment thereof, may comprise an LCDR1, an LCDR2, and an LCDR3 comprising the amino acid sequences of SEQ ID NOs: 23, 136, and 249, respectively; and an HCDR1, an HCDR2, and an HCDR3 comprising the amino acid sequences of SEQ ID NOs: 367, 480, and 596, respectively. In some embodiments, the anti-CD3 antibody, or antigen-binding fragment thereof, comprises the VL of antibody ABL-CD3-23. The VL may comprise the amino acid sequence of SEQ ID NO: 710. In some embodiments, the anti-CD3 antibody, or antigen-binding fragment thereof, comprises the VH of antibody ABL-CD3-23. The VH may comprise the amino acid sequence of SEQ ID NO: 937. In some embodiments, the anti-CD3 antibody, or antigen-binding fragment thereof, comprises the VL and VH of antibody ABL-CD3-23. In some embodiments, the anti-CD3 antibody, or antigen-binding fragment thereof, comprises a VL comprising the amino acid sequence of SEQ ID NO: 710 and a VH comprising the amino acid sequence of SEQ ID NO: 937. In some embodiments, the anti-CD3 antibody, or antigen-binding fragment thereof, comprises the VL of antibody ABL-CD3-23A. The VL may comprise the amino acid sequence of SEQ ID NO: 823. In some embodiments, the anti-CD3 antibody, or antigen-binding fragment thereof, comprises the VH of antibody ABL-CD3-23A. The VH may comprise the amino acid sequence of SEQ ID NO: 1050. In some embodiments, the anti-CD3 antibody, or antigen-binding fragment thereof, comprises the VL and VH of antibody ABL-CD3-23A. In some embodiments, the anti-CD3 antibody, or antigen-binding fragment thereof, comprises a VL comprising the amino acid sequence of SEQ ID NO: 823 and a VH comprising the amino acid sequence of SEQ ID NO: 1050. In some embodiments, the anti-CD3 antibody, or antigen-binding fragment thereof, comprises ABL-CD3-23_scFv_VH-VL. In some embodiments, the anti-CD3 antibody, or antigen-binding fragment thereof, comprises, from N-terminus to C-terminus, a VH comprising the amino acid sequence of SEQ ID NO: 937; a linker comprising the amino acid sequence of (Gly-Gly-Gly-Gly-Ser)n (SEQ ID NO: 1146), wherein n is 4; and a VL comprising the amino acid sequence of SEQ ID NO: 710. In some embodiments, the anti-CD3 antibody, or antigen-binding fragment thereof, comprises ABL-CD3-23_scFv_VL-VH. In some embodiments, the anti-CD3 antibody, or antigen-binding fragment thereof, comprises, from N-terminus to C-terminus, a VL comprising the amino acid sequence of SEQ ID NO: 710; a linker comprising the amino acid sequence of (Gly-Gly-Gly-Gly-Ser)n (SEQ ID NO: 1146), wherein n is 4; and a VH comprising the amino acid sequence of SEQ ID NO: 937. In some embodiments, the anti-CD3 antibody, or antigen-binding fragment thereof, comprises ABL-CD3-23A_scFv_VH-VL. In some embodiments, the anti-CD3 antibody, or antigen-binding fragment thereof, comprises, from N-terminus to C-terminus, a VH comprising the amino acid sequence of SEQ ID NO: 1050; a linker comprising the amino acid sequence of (Gly-Gly-Gly-Gly-Ser)n (SEQ ID NO: 1146), wherein n is 4; and a VL comprising the amino acid sequence of SEQ ID NO: 823. In some embodiments, the anti-CD3 antibody, or antigen-binding fragment thereof, comprises ABL-CD3-23A_scFv_VL-VH. In some embodiments, the anti-CD3 antibody, or antigen-binding fragment thereof, comprises, from N-terminus to C-terminus, a VL comprising the amino acid sequence of SEQ ID NO: 823; a linker comprising the amino acid sequence of (Gly-Gly-Gly-Gly-Ser)n (SEQ ID NO: 1146), wherein n is 4; and a VH comprising the amino acid sequence of SEQ ID NO: 1050.

In some embodiments, the anti-CD3 antibody, or antigen-binding fragment thereof, comprises the six CDRs of antibody ABL-CD3-24 and ABL-CD3-24A. The anti-CD3 antibody, or antigen-binding fragment thereof, may comprise an LCDR1, an LCDR2, and an LCDR3 comprising the amino acid sequences of SEQ ID NOs: 24, 137, and 250, respectively; and an HCDR1, an HCDR2, and an HCDR3 comprising the amino acid sequences of SEQ ID NOs: 368, 481, and 597, respectively. In some embodiments, the anti-CD3 antibody, or antigen-binding fragment thereof, comprises the VL of antibody ABL-CD3-24. The VL may comprise the amino acid sequence of SEQ ID NO: 711. In some embodiments, the anti-CD3 antibody, or antigen-binding fragment thereof, comprises the VH of antibody ABL-CD3-24. The VH may comprise the amino acid sequence of SEQ ID NO: 938. In some embodiments, the anti-CD3 antibody, or antigen-binding fragment thereof, comprises the VL and VH of antibody ABL-CD3-24. In some embodiments, the anti-CD3 antibody, or antigen-binding fragment thereof, comprises a VL comprising the amino acid sequence of SEQ ID NO: 711 and a VH comprising the amino acid sequence of SEQ ID NO: 938. In some embodiments, the anti-CD3 antibody, or antigen-binding fragment thereof, comprises the VL of antibody ABL-CD3-24A. The VL may comprise the amino acid sequence of SEQ ID NO: 824. In some embodiments, the anti-CD3 antibody, or antigen-binding fragment thereof, comprises the VH of antibody ABL-CD3-24A. The VH may comprise the amino acid sequence of SEQ ID NO: 1051. In some embodiments, the anti-CD3 antibody, or antigen-binding fragment thereof, comprises the VL and VH of antibody ABL-CD3-24A. In some embodiments, the anti-CD3 antibody, or antigen-binding fragment thereof, comprises a VL comprising the amino acid sequence of SEQ ID NO: 824 and a VH comprising the amino acid sequence of SEQ ID NO: 1051. In some embodiments, the anti-CD3 antibody, or antigen-binding fragment thereof, comprises ABL-CD3-24_scFv_VH-VL. In some embodiments, the anti-CD3 antibody, or antigen-binding fragment thereof, comprises, from N-terminus to C-terminus, a VH comprising the amino acid sequence of SEQ ID NO: 938; a linker comprising the amino acid sequence of (Gly-Gly-Gly-Gly-Ser)n (SEQ ID NO: 1146), wherein n is 4; and a VL comprising the amino acid sequence of SEQ ID NO: 711. In some embodiments, the anti-CD3 antibody, or antigen-binding fragment thereof, comprises ABL-CD3-24_scFv_VL-VH. In some embodiments, the anti-CD3 antibody, or antigen-binding fragment thereof, comprises, from N-terminus to C-terminus, a VL comprising the amino acid sequence of SEQ ID NO: 711; a linker comprising the amino acid sequence of (Gly-Gly-Gly-Gly-Ser)n (SEQ ID NO: 1146), wherein n is 4; and a VH comprising the amino acid sequence of SEQ ID NO: 938. In some embodiments, the anti-CD3 antibody, or antigen-binding fragment thereof, comprises ABL-CD3-24A_scFv_VH-VL. In some embodiments, the anti-CD3 antibody, or antigen-binding fragment thereof, comprises, from N-terminus to C-terminus, a VH comprising the amino acid sequence of SEQ ID NO: 1051; a linker comprising the amino acid sequence of (Gly-Gly-Gly-Gly-Ser)n (SEQ ID NO: 1146), wherein n is 4; and a VL comprising the amino acid sequence of SEQ ID NO: 824. In some embodiments, the anti-CD3 antibody, or antigen-binding fragment thereof, comprises ABL-CD3-24A_scFv_VL-VH. In some embodiments, the anti-CD3 antibody, or antigen-binding fragment thereof, comprises, from N-terminus to C-terminus, a VL comprising the amino acid sequence of SEQ ID NO: 824; a linker comprising the amino acid sequence of (Gly-Gly-Gly-Gly-Ser)n (SEQ ID NO: 1146), wherein n is 4; and a VH comprising the amino acid sequence of SEQ ID NO: 1051.

In some embodiments, the anti-CD3 antibody, or antigen-binding fragment thereof, comprises the six CDRs of antibody ABL-CD3-25 and ABL-CD3-25A. The anti-CD3 antibody, or antigen-binding fragment thereof, may comprise an LCDR1, an LCDR2, and an LCDR3 comprising the amino acid sequences of SEQ ID NOs: 25, 138, and 251, respectively; and an HCDR1, an HCDR2, and an HCDR3 comprising the amino acid sequences of SEQ ID NOs: 369, 482, and 598, respectively. In some embodiments, the anti-CD3 antibody, or antigen-binding fragment thereof, comprises the VL of antibody ABL-CD3-25. The VL may comprise the amino acid sequence of SEQ ID NO: 712. In some embodiments, the anti-CD3 antibody, or antigen-binding fragment thereof, comprises the VH of antibody ABL-CD3-25. The VH may comprise the amino acid sequence of SEQ ID NO: 939. In some embodiments, the anti-CD3 antibody, or antigen-binding fragment thereof, comprises the VL and VH of antibody ABL-CD3-25. In some embodiments, the anti-CD3 antibody, or antigen-binding fragment thereof, comprises a VL comprising the amino acid sequence of SEQ ID NO: 712 and a VH comprising the amino acid sequence of SEQ ID NO: 939. In some embodiments, the anti-CD3 antibody, or antigen-binding fragment thereof, comprises the VL of antibody ABL-CD3-25A. The VL may comprise the amino acid sequence of SEQ ID NO: 825. In some embodiments, the anti-CD3 antibody, or antigen-binding fragment thereof, comprises the VH of antibody ABL-CD3-25A. The VH may comprise the amino acid sequence of SEQ ID NO: 1052. In some embodiments, the anti-CD3 antibody, or antigen-binding fragment thereof, comprises the VL and VH of antibody ABL-CD3-25A. In some embodiments, the anti-CD3 antibody, or antigen-binding fragment thereof, comprises a VL comprising the amino acid sequence of SEQ ID NO: 825 and a VH comprising the amino acid sequence of SEQ ID NO: 1052. In some embodiments, the anti-CD3 antibody, or antigen-binding fragment thereof, comprises ABL-CD3-25_scFv_VH-VL. In some embodiments, the anti-CD3 antibody, or antigen-binding fragment thereof, comprises, from N-terminus to C-terminus, a VH comprising the amino acid sequence of SEQ ID NO: 939; a linker comprising the amino acid sequence of (Gly-Gly-Gly-Gly-Ser)n (SEQ ID NO: 1146), wherein n is 4; and a VL comprising the amino acid sequence of SEQ ID NO: 712. In some embodiments, the anti-CD3 antibody, or antigen-binding fragment thereof, comprises ABL-CD3-25_scFv_VL-VH. In some embodiments, the anti-CD3 antibody, or antigen-binding fragment thereof, comprises, from N-terminus to C-terminus, a VL comprising the amino acid sequence of SEQ ID NO: 712; a linker comprising the amino acid sequence of (Gly-Gly-Gly-Gly-Ser)n (SEQ ID NO: 1146), wherein n is 4; and a VH comprising the amino acid sequence of SEQ ID NO: 939. In some embodiments, the anti-CD3 antibody, or antigen-binding fragment thereof, comprises ABL-CD3-25A_scFv_VH-VL. In some embodiments, the anti-CD3 antibody, or antigen-binding fragment thereof, comprises, from N-terminus to C-terminus, a VH comprising the amino acid sequence of SEQ ID NO: 1052; a linker comprising the amino acid sequence of (Gly-Gly-Gly-Gly-Ser)n (SEQ ID NO: 1146), wherein n is 4; and a VL comprising the amino acid sequence of SEQ ID NO: 825. In some embodiments, the anti-CD3 antibody, or antigen-binding fragment thereof, comprises ABL-CD3-25A_scFv_VL-VH. In some embodiments, the anti-CD3 antibody, or antigen-binding fragment thereof, comprises, from N-terminus to C-terminus, a VL comprising the amino acid sequence of SEQ ID NO: 825; a linker comprising the amino acid sequence of (Gly-Gly-Gly-Gly-Ser)n (SEQ ID NO: 1146), wherein n is 4; and a VH comprising the amino acid sequence of SEQ ID NO: 1052.

In some embodiments, the anti-CD3 antibody, or antigen-binding fragment thereof, comprises the six CDRs of antibody ABL-CD3-26 and ABL-CD3-26A. The anti-CD3 antibody, or antigen-binding fragment thereof, may comprise an LCDR1, an LCDR2, and an LCDR3 comprising the amino acid sequences of SEQ ID NOs: 26, 139, and 252, respectively; and an HCDR1, an HCDR2, and an HCDR3 comprising the amino acid sequences of SEQ ID NOs: 370, 483, and 599, respectively. In some embodiments, the anti-CD3 antibody, or antigen-binding fragment thereof, comprises the VL of antibody ABL-CD3-26. The VL may comprise the amino acid sequence of SEQ ID NO: 713. In some embodiments, the anti-CD3 antibody, or antigen-binding fragment thereof, comprises the VH of antibody ABL-CD3-26. The VH may comprise the amino acid sequence of SEQ ID NO: 940. In some embodiments, the anti-CD3 antibody, or antigen-binding fragment thereof, comprises the VL and VH of antibody ABL-CD3-26. In some embodiments, the anti-CD3 antibody, or antigen-binding fragment thereof, comprises a VL comprising the amino acid sequence of SEQ ID NO: 713 and a VH comprising the amino acid sequence of SEQ ID NO: 940. In some embodiments, the anti-CD3 antibody, or antigen-binding fragment thereof, comprises the VL of antibody ABL-CD3-26A. The VL may comprise the amino acid sequence of SEQ ID NO: 826. In some embodiments, the anti-CD3 antibody, or antigen-binding fragment thereof, comprises the VH of antibody ABL-CD3-26A. The VH may comprise the amino acid sequence of SEQ ID NO: 1053. In some embodiments, the anti-CD3 antibody, or antigen-binding fragment thereof, comprises the VL and VH of antibody ABL-CD3-26A. In some embodiments, the anti-CD3 antibody, or antigen-binding fragment thereof, comprises a VL comprising the amino acid sequence of SEQ ID NO: 826 and a VH comprising the amino acid sequence of SEQ ID NO: 1053. In some embodiments, the anti-CD3 antibody, or antigen-binding fragment thereof, comprises ABL-CD3-26_scFv_VH-VL. In some embodiments, the anti-CD3 antibody, or antigen-binding fragment thereof, comprises, from N-terminus to C-terminus, a VH comprising the amino acid sequence of SEQ ID NO: 940; a linker comprising the amino acid sequence of (Gly-Gly-Gly-Gly-Ser)n (SEQ ID NO: 1146), wherein n is 4; and a VL comprising the amino acid sequence of SEQ ID NO: 713. In some embodiments, the anti-CD3 antibody, or antigen-binding fragment thereof, comprises ABL-CD3-26_scFv_VL-VH. In some embodiments, the anti-CD3 antibody, or antigen-binding fragment thereof, comprises, from N-terminus to C-terminus, a VL comprising the amino acid sequence of SEQ ID NO: 713; a linker comprising the amino acid sequence of (Gly-Gly-Gly-Gly-Ser)n (SEQ ID NO: 1146), wherein n is 4; and a VH comprising the amino acid sequence of SEQ ID NO: 940. In some embodiments, the anti-CD3 antibody, or antigen-binding fragment thereof, comprises ABL-CD3-26A_scFv_VH-VL. In some embodiments, the anti-CD3 antibody, or antigen-binding fragment thereof, comprises, from N-terminus to C-terminus, a VH comprising the amino acid sequence of SEQ ID NO: 1053; a linker comprising the amino acid sequence of (Gly-Gly-Gly-Gly-Ser)n (SEQ ID NO: 1146), wherein n is 4; and a VL comprising the amino acid sequence of SEQ ID NO: 826. In some embodiments, the anti-CD3 antibody, or antigen-binding fragment thereof, comprises ABL-CD3-26A_scFv_VL-VH. In some embodiments, the anti-CD3 antibody, or antigen-binding fragment thereof, comprises, from N-terminus to C-terminus, a VL comprising the amino acid sequence of SEQ ID NO: 826; a linker comprising the amino acid sequence of (Gly-Gly-Gly-Gly-Ser)n (SEQ ID NO: 1146), wherein n is 4; and a VH comprising the amino acid sequence of SEQ ID NO: 1053.

In some embodiments, the anti-CD3 antibody, or antigen-binding fragment thereof, comprises the six CDRs of antibody ABL-CD3-27. The anti-CD3 antibody, or antigen-binding fragment thereof, may comprise an LCDR1, an LCDR2, and an LCDR3 comprising the amino acid sequences of SEQ ID NOs: 27, 140, and 253, respectively; and an HCDR1, an HCDR2, and an HCDR3 comprising the amino acid sequences of SEQ ID NOs: 371, 484, and 600, respectively. In some embodiments, the anti-CD3 antibody, or antigen-binding fragment thereof, comprises the six CDRs of antibody ABL-CD3-27A. The anti-CD3 antibody, or antigen-binding fragment thereof, may comprise an LCDR1, an LCDR2, and an LCDR3 comprising the amino acid sequences of SEQ ID NOs: 27, 140, and 253, respectively; and an HCDR1, an HCDR2, and an HCDR3 comprising the amino acid sequences of SEQ ID NOs: 371, 484, and 687, respectively. In some embodiments, the anti-CD3 antibody, or antigen-binding fragment thereof, comprises the VL of antibody ABL-CD3-27. The VL may comprise the amino acid sequence of SEQ ID NO: 714. In some embodiments, the anti-CD3 antibody, or antigen-binding fragment thereof, comprises the VH of antibody ABL-CD3-27. The VH may comprise the amino acid sequence of SEQ ID NO: 941. In some embodiments, the anti-CD3 antibody, or antigen-binding fragment thereof, comprises the VL and VH of antibody ABL-CD3-27. In some embodiments, the anti-CD3 antibody, or antigen-binding fragment thereof, comprises a VL comprising the amino acid sequence of SEQ ID NO: 714 and a VH comprising the amino acid sequence of SEQ ID NO: 941. In some embodiments, the anti-CD3 antibody, or antigen-binding fragment thereof, comprises the VL of antibody ABL-CD3-27A. The VL may comprise the amino acid sequence of SEQ ID NO: 827. In some embodiments, the anti-CD3 antibody, or antigen-binding fragment thereof, comprises the VH of antibody ABL-CD3-27A. The VH may comprise the amino acid sequence of SEQ ID NO: 1054. In some embodiments, the anti-CD3 antibody, or antigen-binding fragment thereof, comprises the VL and VH of antibody ABL-CD3-27A. In some embodiments, the anti-CD3 antibody, or antigen-binding fragment thereof, comprises a VL comprising the amino acid sequence of SEQ ID NO: 827 and a VH comprising the amino acid sequence of SEQ ID NO: 1054. In some embodiments, the anti-CD3 antibody, or antigen-binding fragment thereof, comprises ABL-CD3-27_scFv_VH-VL. In some embodiments, the anti-CD3 antibody, or antigen-binding fragment thereof, comprises, from N-terminus to C-terminus, a VH comprising the amino acid sequence of SEQ ID NO: 941; a linker comprising the amino acid sequence of (Gly-Gly-Gly-Gly-Ser)n (SEQ ID NO: 1146), wherein n is 4; and a VL comprising the amino acid sequence of SEQ ID NO: 714. In some embodiments, the anti-CD3 antibody, or antigen-binding fragment thereof, comprises ABL-CD3-27_scFv_VL-VH. In some embodiments, the anti-CD3 antibody, or antigen-binding fragment thereof, comprises, from N-terminus to C-terminus, a VL comprising the amino acid sequence of SEQ ID NO: 714; a linker comprising the amino acid sequence of (Gly-Gly-Gly-Gly-Ser)n (SEQ ID NO: 1146), wherein n is 4; and a VH comprising the amino acid sequence of SEQ ID NO: 941. In some embodiments, the anti-CD3 antibody, or antigen-binding fragment thereof, comprises ABL-CD3-27A_scFv_VH-VL. In some embodiments, the anti-CD3 antibody, or antigen-binding fragment thereof, comprises the amino acid sequence of SEQ ID NO: 1159. In some embodiments, the anti-CD3 antibody, or antigen-binding fragment thereof, comprises ABL-CD3-27A_scFv_VL-VH. In some embodiments, the anti-CD3 antibody, or antigen-binding fragment thereof, comprises the amino acid sequence of SEQ ID NO: 1200.

In some embodiments, the anti-CD3 antibody, or antigen-binding fragment thereof, comprises the six CDRs of antibody ABL-CD3-28 and ABL-CD3-28A. The anti-CD3 antibody, or antigen-binding fragment thereof, may comprise an LCDR1, an LCDR2, and an LCDR3 comprising the amino acid sequences of SEQ ID NOs: 28, 141, and 254, respectively; and an HCDR1, an HCDR2, and an HCDR3 comprising the amino acid sequences of SEQ ID NOs: 372, 485, and 601, respectively. In some embodiments, the anti-CD3 antibody, or antigen-binding fragment thereof, comprises the VL of antibody ABL-CD3-28. The VL may comprise the amino acid sequence of SEQ ID NO: 715. In some embodiments, the anti-CD3 antibody, or antigen-binding fragment thereof, comprises the VH of antibody ABL-CD3-28. The VH may comprise the amino acid sequence of SEQ ID NO: 942. In some embodiments, the anti-CD3 antibody, or antigen-binding fragment thereof, comprises the VL and VH of antibody ABL-CD3-28. In some embodiments, the anti-CD3 antibody, or antigen-binding fragment thereof, comprises a VL comprising the amino acid sequence of SEQ ID NO: 715 and a VH comprising the amino acid sequence of SEQ ID NO: 942. In some embodiments, the anti-CD3 antibody, or antigen-binding fragment thereof, comprises the VL of antibody ABL-CD3-28A. The VL may comprise the amino acid sequence of SEQ ID NO: 828. In some embodiments, the anti-CD3 antibody, or antigen-binding fragment thereof, comprises the VH of antibody ABL-CD3-28A. The VH may comprise the amino acid sequence of SEQ ID NO: 1055. In some embodiments, the anti-CD3 antibody, or antigen-binding fragment thereof, comprises the VL and VH of antibody ABL-CD3-28A. In some embodiments, the anti-CD3 antibody, or antigen-binding fragment thereof, comprises a VL comprising the amino acid sequence of SEQ ID NO: 828 and a VH comprising the amino acid sequence of SEQ ID NO: 1055. In some embodiments, the anti-CD3 antibody, or antigen-binding fragment thereof, comprises ABL-CD3-28_scFv_VH-VL. In some embodiments, the anti-CD3 antibody, or antigen-binding fragment thereof, comprises, from N-terminus to C-terminus, a VH comprising the amino acid sequence of SEQ ID NO: 942; a linker comprising the amino acid sequence of (Gly-Gly-Gly-Gly-Ser)n (SEQ ID NO: 1146), wherein n is 4; and a VL comprising the amino acid sequence of SEQ ID NO: 715. In some embodiments, the anti-CD3 antibody, or antigen-binding fragment thereof, comprises ABL-CD3-28_scFv_VL-VH. In some embodiments, the anti-CD3 antibody, or antigen-binding fragment thereof, comprises, from N-terminus to C-terminus, a VL comprising the amino acid sequence of SEQ ID NO: 715; a linker comprising the amino acid sequence of (Gly-Gly-Gly-Gly-Ser)n (SEQ ID NO: 1146), wherein n is 4; and a VH comprising the amino acid sequence of SEQ ID NO: 942. In some embodiments, the anti-CD3 antibody, or antigen-binding fragment thereof, comprises ABL-CD3-28A_scFv_VH-VL. In some embodiments, the anti-CD3 antibody, or antigen-binding fragment thereof, comprises, from N-terminus to C-terminus, a VH comprising the amino acid sequence of SEQ ID NO: 1055; a linker comprising the amino acid sequence of (Gly-Gly-Gly-Gly-Ser)n (SEQ ID NO: 1146), wherein n is 4; and a VL comprising the amino acid sequence of SEQ ID NO: 828. In some embodiments, the anti-CD3 antibody, or antigen-binding fragment thereof, comprises ABL-CD3-28A_scFv_VL-VH. In some embodiments, the anti-CD3 antibody, or antigen-binding fragment thereof, comprises, from N-terminus to C-terminus, a VL comprising the amino acid sequence of SEQ ID NO: 828; a linker comprising the amino acid sequence of (Gly-Gly-Gly-Gly-Ser)n (SEQ ID NO: 1146), wherein n is 4; and a VH comprising the amino acid sequence of SEQ ID NO: 1055.

In some embodiments, the anti-CD3 antibody, or antigen-binding fragment thereof, comprises the six CDRs of antibody ABL-CD3-29 and ABL-CD3-29A. The anti-CD3 antibody, or antigen-binding fragment thereof, may comprise an LCDR1, an LCDR2, and an LCDR3 comprising the amino acid sequences of SEQ ID NOs: 29, 142, and 255, respectively; and an HCDR1, an HCDR2, and an HCDR3 comprising the amino acid sequences of SEQ ID NOs: 373, 486, and 602, respectively. In some embodiments, the anti-CD3 antibody, or antigen-binding fragment thereof, comprises the VL of antibody ABL-CD3-29. The VL may comprise the amino acid sequence of SEQ ID NO: 716. In some embodiments, the anti-CD3 antibody, or antigen-binding fragment thereof, comprises the VH of antibody ABL-CD3-29. The VH may comprise the amino acid sequence of SEQ ID NO: 943. In some embodiments, the anti-CD3 antibody, or antigen-binding fragment thereof, comprises the VL and VH of antibody ABL-CD3-29. In some embodiments, the anti-CD3 antibody, or antigen-binding fragment thereof, comprises a VL comprising the amino acid sequence of SEQ ID NO: 716 and a VH comprising the amino acid sequence of SEQ ID NO: 943. In some embodiments, the anti-CD3 antibody, or antigen-binding fragment thereof, comprises the VL of antibody ABL-CD3-29A. The VL may comprise the amino acid sequence of SEQ ID NO: 829. In some embodiments, the anti-CD3 antibody, or antigen-binding fragment thereof, comprises the VH of antibody ABL-CD3-29A. The VH may comprise the amino acid sequence of SEQ ID NO: 1056. In some embodiments, the anti-CD3 antibody, or antigen-binding fragment thereof, comprises the VL and VH of antibody ABL-CD3-29A. In some embodiments, the anti-CD3 antibody, or antigen-binding fragment thereof, comprises a VL comprising the amino acid sequence of SEQ ID NO: 829 and a VH comprising the amino acid sequence of SEQ ID NO: 1056. In some embodiments, the anti-CD3 antibody, or antigen-binding fragment thereof, comprises ABL-CD3-29_scFv_VH-VL. In some embodiments, the anti-CD3 antibody, or antigen-binding fragment thereof, comprises, from N-terminus to C-terminus, a VH comprising the amino acid sequence of SEQ ID NO: 943; a linker comprising the amino acid sequence of (Gly-Gly-Gly-Gly-Ser)n (SEQ ID NO: 1146), wherein n is 4; and a VL comprising the amino acid sequence of SEQ ID NO: 716. In some embodiments, the anti-CD3 antibody, or antigen-binding fragment thereof, comprises ABL-CD3-29_scFv_VL-VH. In some embodiments, the anti-CD3 antibody, or antigen-binding fragment thereof, comprises, from N-terminus to C-terminus, a VL comprising the amino acid sequence of SEQ ID NO: 716; a linker comprising the amino acid sequence of (Gly-Gly-Gly-Gly-Ser)n (SEQ ID NO: 1146), wherein n is 4; and a VH comprising the amino acid sequence of SEQ ID NO: 943. In some embodiments, the anti-CD3 antibody, or antigen-binding fragment thereof, comprises ABL-CD3-29A_scFv_VH-VL. In some embodiments, the anti-CD3 antibody, or antigen-binding fragment thereof, comprises the amino acid sequence of SEQ ID NO: 1160. In some embodiments, the anti-CD3 antibody, or antigen-binding fragment thereof, comprises ABL-CD3-29A_scFv_VL-VH. In some embodiments, the anti-CD3 antibody, or antigen-binding fragment thereof, comprises the amino acid sequence of SEQ ID NO: 1201.

In some embodiments, the anti-CD3 antibody, or antigen-binding fragment thereof, comprises the six CDRs of antibody ABL-CD3-30 and ABL-CD3-30A. The anti-CD3 antibody, or antigen-binding fragment thereof, may comprise an LCDR1, an LCDR2, and an LCDR3 comprising the amino acid sequences of SEQ ID NOs: 30, 143, and 256, respectively; and an HCDR1, an HCDR2, and an HCDR3 comprising the amino acid sequences of SEQ ID NOs: 374, 487, and 603, respectively. In some embodiments, the anti-CD3 antibody, or antigen-binding fragment thereof, comprises the VL of antibody ABL-CD3-30. The VL may comprise the amino acid sequence of SEQ ID NO: 717. In some embodiments, the anti-CD3 antibody, or antigen-binding fragment thereof, comprises the VH of antibody ABL-CD3-30. The VH may comprise the amino acid sequence of SEQ ID NO: 944. In some embodiments, the anti-CD3 antibody, or antigen-binding fragment thereof, comprises the VL and VH of antibody ABL-CD3-30. In some embodiments, the anti-CD3 antibody, or antigen-binding fragment thereof, comprises a VL comprising the amino acid sequence of SEQ ID NO: 717 and a VH comprising the amino acid sequence of SEQ ID NO: 944. In some embodiments, the anti-CD3 antibody, or antigen-binding fragment thereof, comprises the VL of antibody ABL-CD3-30A. The VL may comprise the amino acid sequence of SEQ ID NO: 830. In some embodiments, the anti-CD3 antibody, or antigen-binding fragment thereof, comprises the VH of antibody ABL-CD3-30A. The VH may comprise the amino acid sequence of SEQ ID NO: 1057. In some embodiments, the anti-CD3 antibody, or antigen-binding fragment thereof, comprises the VL and VH of antibody ABL-CD3-30A. In some embodiments, the anti-CD3 antibody, or antigen-binding fragment thereof, comprises a VL comprising the amino acid sequence of SEQ ID NO: 830 and a VH comprising the amino acid sequence of SEQ ID NO: 1057. In some embodiments, the anti-CD3 antibody, or antigen-binding fragment thereof, comprises ABL-CD3-30_scFv_VH-VL. In some embodiments, the anti-CD3 antibody, or antigen-binding fragment thereof, comprises, from N-terminus to C-terminus, a VH comprising the amino acid sequence of SEQ ID NO: 944; a linker comprising the amino acid sequence of (Gly-Gly-Gly-Gly-Ser)n (SEQ ID NO: 1146), wherein n is 4; and a VL comprising the amino acid sequence of SEQ ID NO: 717. In some embodiments, the anti-CD3 antibody, or antigen-binding fragment thereof, comprises ABL-CD3-30_scFv_VL-VH. In some embodiments, the anti-CD3 antibody, or antigen-binding fragment thereof, comprises, from N-terminus to C-terminus, a VL comprising the amino acid sequence of SEQ ID NO: 717; a linker comprising the amino acid sequence of (Gly-Gly-Gly-Gly-Ser)n (SEQ ID NO: 1146), wherein n is 4; and a VH comprising the amino acid sequence of SEQ ID NO: 944. In some embodiments, the anti-CD3 antibody, or antigen-binding fragment thereof, comprises ABL-CD3-30A_scFv_VH-VL. In some embodiments, the anti-CD3 antibody, or antigen-binding fragment thereof, comprises, from N-terminus to C-terminus, a VH comprising the amino acid sequence of SEQ ID NO: 1057; a linker comprising the amino acid sequence of (Gly-Gly-Gly-Gly-Ser)n (SEQ ID NO: 1146), wherein n is 4; and a VL comprising the amino acid sequence of SEQ ID NO: 830. In some embodiments, the anti-CD3 antibody, or antigen-binding fragment thereof, comprises ABL-CD3-30A_scFv_VL-VH. In some embodiments, the anti-CD3 antibody, or antigen-binding fragment thereof, comprises, from N-terminus to C-terminus, a VL comprising the amino acid sequence of SEQ ID NO: 830; a linker comprising the amino acid sequence of (Gly-Gly-Gly-Gly-Ser)n (SEQ ID NO: 1146), wherein n is 4; and a VH comprising the amino acid sequence of SEQ ID NO: 1057.

In some embodiments, the anti-CD3 antibody, or antigen-binding fragment thereof, comprises the six CDRs of antibody ABL-CD3-31 and ABL-CD3-31A. The anti-CD3 antibody, or antigen-binding fragment thereof, may comprise an LCDR1, an LCDR2, and an LCDR3 comprising the amino acid sequences of SEQ ID NOs: 31, 144, and 257, respectively; and an HCDR1, an HCDR2, and an HCDR3 comprising the amino acid sequences of SEQ ID NOs: 375, 488, and 604, respectively. In some embodiments, the anti-CD3 antibody, or antigen-binding fragment thereof, comprises the VL of antibody ABL-CD3-31. The VL may comprise the amino acid sequence of SEQ ID NO: 718. In some embodiments, the anti-CD3 antibody, or antigen-binding fragment thereof, comprises the VH of antibody ABL-CD3-31. The VH may comprise the amino acid sequence of SEQ ID NO: 945. In some embodiments, the anti-CD3 antibody, or antigen-binding fragment thereof, comprises the VL and VH of antibody ABL-CD3-31. In some embodiments, the anti-CD3 antibody, or antigen-binding fragment thereof, comprises a VL comprising the amino acid sequence of SEQ ID NO: 718 and a VH comprising the amino acid sequence of SEQ ID NO: 945. In some embodiments, the anti-CD3 antibody, or antigen-binding fragment thereof, comprises the VL of antibody ABL-CD3-31A. The VL may comprise the amino acid sequence of SEQ ID NO: 831. In some embodiments, the anti-CD3 antibody, or antigen-binding fragment thereof, comprises the VH of antibody ABL-CD3-31A. The VH may comprise the amino acid sequence of SEQ ID NO: 1058. In some embodiments, the anti-CD3 antibody, or antigen-binding fragment thereof, comprises the VL and VH of antibody ABL-CD3-31A. In some embodiments, the anti-CD3 antibody, or antigen-binding fragment thereof, comprises a VL comprising the amino acid sequence of SEQ ID NO: 831 and a VH comprising the amino acid sequence of SEQ ID NO: 1058. In some embodiments, the anti-CD3 antibody, or antigen-binding fragment thereof, comprises ABL-CD3-31_scFv_VH-VL. In some embodiments, the anti-CD3 antibody, or antigen-binding fragment thereof, comprises, from N-terminus to C-terminus, a VH comprising the amino acid sequence of SEQ ID NO: 945; a linker comprising the amino acid sequence of (Gly-Gly-Gly-Gly-Ser)n (SEQ ID NO: 1146), wherein n is 4; and a VL comprising the amino acid sequence of SEQ ID NO: 718. In some embodiments, the anti-CD3 antibody, or antigen-binding fragment thereof, comprises ABL-CD3-31_scFv_VL-VH. In some embodiments, the anti-CD3 antibody, or antigen-binding fragment thereof, comprises, from N-terminus to C-terminus, a VL comprising the amino acid sequence of SEQ ID NO: 718; a linker comprising the amino acid sequence of (Gly-Gly-Gly-Gly-Ser)n (SEQ ID NO: 1146), wherein n is 4; and a VH comprising the amino acid sequence of SEQ ID NO: 945. In some embodiments, the anti-CD3 antibody, or antigen-binding fragment thereof, comprises ABL-CD3-31A_scFv_VH-VL. In some embodiments, the anti-CD3 antibody, or antigen-binding fragment thereof, comprises the amino acid sequence of SEQ ID NO: 1161. In some embodiments, the anti-CD3 antibody, or antigen-binding fragment thereof, comprises ABL-CD3-31A_scFv_VL-VH. In some embodiments, the anti-CD3 antibody, or antigen-binding fragment thereof, comprises the amino acid sequence of SEQ ID NO: 1202.

In some embodiments, the anti-CD3 antibody, or antigen-binding fragment thereof, comprises the six CDRs of antibody ABL-CD3-32 and ABL-CD3-32A. The anti-CD3 antibody, or antigen-binding fragment thereof, may comprise an LCDR1, an LCDR2, and an LCDR3 comprising the amino acid sequences of SEQ ID NOs: 32, 145, and 258, respectively; and an HCDR1, an HCDR2, and an HCDR3 comprising the amino acid sequences of SEQ ID NOs: 376, 489, and 605, respectively. In some embodiments, the anti-CD3 antibody, or antigen-binding fragment thereof, comprises the VL of antibody ABL-CD3-32. The VL may comprise the amino acid sequence of SEQ ID NO: 719. In some embodiments, the anti-CD3 antibody, or antigen-binding fragment thereof, comprises the VH of antibody ABL-CD3-32. The VH may comprise the amino acid sequence of SEQ ID NO: 946. In some embodiments, the anti-CD3 antibody, or antigen-binding fragment thereof, comprises the VL and VH of antibody ABL-CD3-32. In some embodiments, the anti-CD3 antibody, or antigen-binding fragment thereof, comprises a VL comprising the amino acid sequence of SEQ ID NO: 719 and a VH comprising the amino acid sequence of SEQ ID NO: 946. In some embodiments, the anti-CD3 antibody, or antigen-binding fragment thereof, comprises the VL of antibody ABL-CD3-32A. The VL may comprise the amino acid sequence of SEQ ID NO: 832. In some embodiments, the anti-CD3 antibody, or antigen-binding fragment thereof, comprises the VH of antibody ABL-CD3-32A. The VH may comprise the amino acid sequence of SEQ ID NO: 1059. In some embodiments, the anti-CD3 antibody, or antigen-binding fragment thereof, comprises the VL and VH of antibody ABL-CD3-32A. In some embodiments, the anti-CD3 antibody, or antigen-binding fragment thereof, comprises a VL comprising the amino acid sequence of SEQ ID NO: 832 and a VH comprising the amino acid sequence of SEQ ID NO: 1059. In some embodiments, the anti-CD3 antibody, or antigen-binding fragment thereof, comprises ABL-CD3-32_scFv_VH-VL. In some embodiments, the anti-CD3 antibody, or antigen-binding fragment thereof, comprises, from N-terminus to C-terminus, a VH comprising the amino acid sequence of SEQ ID NO: 946; a linker comprising the amino acid sequence of (Gly-Gly-Gly-Gly-Ser)n (SEQ ID NO: 1146), wherein n is 4; and a VL comprising the amino acid sequence of SEQ ID NO: 719. In some embodiments, the anti-CD3 antibody, or antigen-binding fragment thereof, comprises ABL-CD3-32_scFv_VL-VH. In some embodiments, the anti-CD3 antibody, or antigen-binding fragment thereof, comprises, from N-terminus to C-terminus, a VL comprising the amino acid sequence of SEQ ID NO: 719; a linker comprising the amino acid sequence of (Gly-Gly-Gly-Gly-Ser)n (SEQ ID NO: 1146), wherein n is 4; and a VH comprising the amino acid sequence of SEQ ID NO: 946. In some embodiments, the anti-CD3 antibody, or antigen-binding fragment thereof, comprises ABL-CD3-32A_scFv_VH-VL. In some embodiments, the anti-CD3 antibody, or antigen-binding fragment thereof, comprises, from N-terminus to C-terminus, a VH comprising the amino acid sequence of SEQ ID NO: 1059; a linker comprising the amino acid sequence of (Gly-Gly-Gly-Gly-Ser)n (SEQ ID NO: 1146), wherein n is 4; and a VL comprising the amino acid sequence of SEQ ID NO: 832. In some embodiments, the anti-CD3 antibody, or antigen-binding fragment thereof, comprises ABL-CD3-32A_scFv_VL-VH. In some embodiments, the anti-CD3 antibody, or antigen-binding fragment thereof, comprises, from N-terminus to C-terminus, a VL comprising the amino acid sequence of SEQ ID NO: 832; a linker comprising the amino acid sequence of (Gly-Gly-Gly-Gly-Ser)n (SEQ ID NO: 1146), wherein n is 4; and a VH comprising the amino acid sequence of SEQ ID NO: 1059.

In some embodiments, the anti-CD3 antibody, or antigen-binding fragment thereof, comprises the six CDRs of antibody ABL-CD3-33 and ABL-CD3-33A. The anti-CD3 antibody, or antigen-binding fragment thereof, may comprise an LCDR1, an LCDR2, and an LCDR3 comprising the amino acid sequences of SEQ ID NOs: 33, 146, and 259, respectively; and an HCDR1, an HCDR2, and an HCDR3 comprising the amino acid sequences of SEQ ID NOs: 377, 490, and 606, respectively. In some embodiments, the anti-CD3 antibody, or antigen-binding fragment thereof, comprises the VL of antibody ABL-CD3-33. The VL may comprise the amino acid sequence of SEQ ID NO: 720. In some embodiments, the anti-CD3 antibody, or antigen-binding fragment thereof, comprises the VH of antibody ABL-CD3-33. The VH may comprise the amino acid sequence of SEQ ID NO: 947. In some embodiments, the anti-CD3 antibody, or antigen-binding fragment thereof, comprises the VL and VH of antibody ABL-CD3-33. In some embodiments, the anti-CD3 antibody, or antigen-binding fragment thereof, comprises a VL comprising the amino acid sequence of SEQ ID NO: 720 and a VH comprising the amino acid sequence of SEQ ID NO: 947. In some embodiments, the anti-CD3 antibody, or antigen-binding fragment thereof, comprises the VL of antibody ABL-CD3-33A. The VL may comprise the amino acid sequence of SEQ ID NO: 833. In some embodiments, the anti-CD3 antibody, or antigen-binding fragment thereof, comprises the VH of antibody ABL-CD3-33A. The VH may comprise the amino acid sequence of SEQ ID NO: 1060. In some embodiments, the anti-CD3 antibody, or antigen-binding fragment thereof, comprises the VL and VH of antibody ABL-CD3-33A. In some embodiments, the anti-CD3 antibody, or antigen-binding fragment thereof, comprises a VL comprising the amino acid sequence of SEQ ID NO: 833 and a VH comprising the amino acid sequence of SEQ ID NO: 1060. In some embodiments, the anti-CD3 antibody, or antigen-binding fragment thereof, comprises ABL-CD3-33_scFv_VH-VL. In some embodiments, the anti-CD3 antibody, or antigen-binding fragment thereof, comprises, from N-terminus to C-terminus, a VH comprising the amino acid sequence of SEQ ID NO: 947; a linker comprising the amino acid sequence of (Gly-Gly-Gly-Gly-Ser)n (SEQ ID NO: 1146), wherein n is 4; and a VL comprising the amino acid sequence of SEQ ID NO: 720. In some embodiments, the anti-CD3 antibody, or antigen-binding fragment thereof, comprises ABL-CD3-33_scFv_VL-VH. In some embodiments, the anti-CD3 antibody, or antigen-binding fragment thereof, comprises, from N-terminus to C-terminus, a VL comprising the amino acid sequence of SEQ ID NO: 720; a linker comprising the amino acid sequence of (Gly-Gly-Gly-Gly-Ser)n (SEQ ID NO: 1146), wherein n is 4; and a VH comprising the amino acid sequence of SEQ ID NO: 947. In some embodiments, the anti-CD3 antibody, or antigen-binding fragment thereof, comprises ABL-CD3-33A_scFv_VH-VL. In some embodiments, the anti-CD3 antibody, or antigen-binding fragment thereof, comprises, from N-terminus to C-terminus, a VH comprising the amino acid sequence of SEQ ID NO: 1060; a linker comprising the amino acid sequence of (Gly-Gly-Gly-Gly-Ser)n (SEQ ID NO: 1146), wherein n is 4; and a VL comprising the amino acid sequence of SEQ ID NO: 833. In some embodiments, the anti-CD3 antibody, or antigen-binding fragment thereof, comprises ABL-CD3-33A_scFv_VL-VH. In some embodiments, the anti-CD3 antibody, or antigen-binding fragment thereof, comprises, from N-terminus to C-terminus, a VL comprising the amino acid sequence of SEQ ID NO: 833; a linker comprising the amino acid sequence of (Gly-Gly-Gly-Gly-Ser)n (SEQ ID NO: 1146), wherein n is 4; and a VH comprising the amino acid sequence of SEQ ID NO: 1060.

In some embodiments, the anti-CD3 antibody, or antigen-binding fragment thereof, comprises the six CDRs of antibody ABL-CD3-34 and ABL-CD3-34A. The anti-CD3 antibody, or antigen-binding fragment thereof, may comprise an LCDR1, an LCDR2, and an LCDR3 comprising the amino acid sequences of SEQ ID NOs: 34, 147, and 260, respectively; and an HCDR1, an HCDR2, and an HCDR3 comprising the amino acid sequences of SEQ ID NOs: 378, 491, and 607, respectively. In some embodiments, the anti-CD3 antibody, or antigen-binding fragment thereof, comprises the VL of antibody ABL-CD3-34. The VL may comprise the amino acid sequence of SEQ ID NO: 721. In some embodiments, the anti-CD3 antibody, or antigen-binding fragment thereof, comprises the VH of antibody ABL-CD3-34. The VH may comprise the amino acid sequence of SEQ ID NO: 948. In some embodiments, the anti-CD3 antibody, or antigen-binding fragment thereof, comprises the VL and VH of antibody ABL-CD3-34. In some embodiments, the anti-CD3 antibody, or antigen-binding fragment thereof, comprises a VL comprising the amino acid sequence of SEQ ID NO: 721 and a VH comprising the amino acid sequence of SEQ ID NO: 948. In some embodiments, the anti-CD3 antibody, or antigen-binding fragment thereof, comprises the VL of antibody ABL-CD3-34A. The VL may comprise the amino acid sequence of SEQ ID NO: 834. In some embodiments, the anti-CD3 antibody, or antigen-binding fragment thereof, comprises the VH of antibody ABL-CD3-34A. The VH may comprise the amino acid sequence of SEQ ID NO: 1061. In some embodiments, the anti-CD3 antibody, or antigen-binding fragment thereof, comprises the VL and VH of antibody ABL-CD3-34A. In some embodiments, the anti-CD3 antibody, or antigen-binding fragment thereof, comprises a VL comprising the amino acid sequence of SEQ ID NO: 834 and a VH comprising the amino acid sequence of SEQ ID NO: 1061. In some embodiments, the anti-CD3 antibody, or antigen-binding fragment thereof, comprises ABL-CD3-34_scFv_VH-VL. In some embodiments, the anti-CD3 antibody, or antigen-binding fragment thereof, comprises, from N-terminus to C-terminus, a VH comprising the amino acid sequence of SEQ ID NO: 948; a linker comprising the amino acid sequence of (Gly-Gly-Gly-Gly-Ser)n (SEQ ID NO: 1146), wherein n is 4; and a VL comprising the amino acid sequence of SEQ ID NO: 721. In some embodiments, the anti-CD3 antibody, or antigen-binding fragment thereof, comprises ABL-CD3-34_scFv_VL-VH. In some embodiments, the anti-CD3 antibody, or antigen-binding fragment thereof, comprises, from N-terminus to C-terminus, a VL comprising the amino acid sequence of SEQ ID NO: 721; a linker comprising the amino acid sequence of (Gly-Gly-Gly-Gly-Ser)n (SEQ ID NO: 1146), wherein n is 4; and a VH comprising the amino acid sequence of SEQ ID NO: 948. In some embodiments, the anti-CD3 antibody, or antigen-binding fragment thereof, comprises ABL-CD3-34A_scFv_VH-VL. In some embodiments, the anti-CD3 antibody, or antigen-binding fragment thereof, comprises the amino acid sequence of SEQ ID NO: 1162. In some embodiments, the anti-CD3 antibody, or antigen-binding fragment thereof, comprises ABL-CD3-34A_scFv_VL-VH. In some embodiments, the anti-CD3 antibody, or antigen-binding fragment thereof, comprises the amino acid sequence of SEQ ID NO: 1203.

In some embodiments, the anti-CD3 antibody, or antigen-binding fragment thereof, comprises the six CDRs of antibody ABL-CD3-35 and ABL-CD3-35A. The anti-CD3 antibody, or antigen-binding fragment thereof, may comprise an LCDR1, an LCDR2, and an LCDR3 comprising the amino acid sequences of SEQ ID NOs: 35, 148, and 261, respectively; and an HCDR1, an HCDR2, and an HCDR3 comprising the amino acid sequences of SEQ ID NOs: 379, 492, and 608, respectively. In some embodiments, the anti-CD3 antibody, or antigen-binding fragment thereof, comprises the VL of antibody ABL-CD3-35. The VL may comprise the amino acid sequence of SEQ ID NO: 722. In some embodiments, the anti-CD3 antibody, or antigen-binding fragment thereof, comprises the VH of antibody ABL-CD3-35. The VH may comprise the amino acid sequence of SEQ ID NO: 949. In some embodiments, the anti-CD3 antibody, or antigen-binding fragment thereof, comprises the VL and VH of antibody ABL-CD3-35. In some embodiments, the anti-CD3 antibody, or antigen-binding fragment thereof, comprises a VL comprising the amino acid sequence of SEQ ID NO: 722 and a VH comprising the amino acid sequence of SEQ ID NO: 949. In some embodiments, the anti-CD3 antibody, or antigen-binding fragment thereof, comprises the VL of antibody ABL-CD3-35A. The VL may comprise the amino acid sequence of SEQ ID NO: 835. In some embodiments, the anti-CD3 antibody, or antigen-binding fragment thereof, comprises the VH of antibody ABL-CD3-35A. The VH may comprise the amino acid sequence of SEQ ID NO: 1062. In some embodiments, the anti-CD3 antibody, or antigen-binding fragment thereof, comprises the VL and VH of antibody ABL-CD3-35A. In some embodiments, the anti-CD3 antibody, or antigen-binding fragment thereof, comprises a VL comprising the amino acid sequence of SEQ ID NO: 835 and a VH comprising the amino acid sequence of SEQ ID NO: 1062. In some embodiments, the anti-CD3 antibody, or antigen-binding fragment thereof, comprises ABL-CD3-35_scFv_VH-VL. In some embodiments, the anti-CD3 antibody, or antigen-binding fragment thereof, comprises, from N-terminus to C-terminus, a VH comprising the amino acid sequence of SEQ ID NO: 949; a linker comprising the amino acid sequence of (Gly-Gly-Gly-Gly-Ser)n (SEQ ID NO: 1146), wherein n is 4; and a VL comprising the amino acid sequence of SEQ ID NO: 722. In some embodiments, the anti-CD3 antibody, or antigen-binding fragment thereof, comprises ABL-CD3-35_scFv_VL-VH. In some embodiments, the anti-CD3 antibody, or antigen-binding fragment thereof, comprises, from N-terminus to C-terminus, a VL comprising the amino acid sequence of SEQ ID NO: 722; a linker comprising the amino acid sequence of (Gly-Gly-Gly-Gly-Ser)n (SEQ ID NO: 1146), wherein n is 4; and a VH comprising the amino acid sequence of SEQ ID NO: 949. In some embodiments, the anti-CD3 antibody, or antigen-binding fragment thereof, comprises ABL-CD3-35A_scFv_VH-VL. In some embodiments, the anti-CD3 antibody, or antigen-binding fragment thereof, comprises, from N-terminus to C-terminus, a VH comprising the amino acid sequence of SEQ ID NO: 1062; a linker comprising the amino acid sequence of (Gly-Gly-Gly-Gly-Ser)n (SEQ ID NO: 1146), wherein n is 4; and a VL comprising the amino acid sequence of SEQ ID NO: 835. In some embodiments, the anti-CD3 antibody, or antigen-binding fragment thereof, comprises ABL-CD3-35A_scFv_VL-VH. In some embodiments, the anti-CD3 antibody, or antigen-binding fragment thereof, comprises, from N-terminus to C-terminus, a VL comprising the amino acid sequence of SEQ ID NO: 835; a linker comprising the amino acid sequence of (Gly-Gly-Gly-Gly-Ser)n (SEQ ID NO: 1146), wherein n is 4; and a VH comprising the amino acid sequence of SEQ ID NO: 1062.

In some embodiments, the anti-CD3 antibody, or antigen-binding fragment thereof, comprises the six CDRs of antibody ABL-CD3-36. The anti-CD3 antibody, or antigen-binding fragment thereof, may comprise an LCDR1, an LCDR2, and an LCDR3 comprising the amino acid sequences of SEQ ID NOs: 36, 149, and 262, respectively; and an HCDR1, an HCDR2, and an HCDR3 comprising the amino acid sequences of SEQ ID NOs: 380, 493, and 609, respectively. In some embodiments, the anti-CD3 antibody, or antigen-binding fragment thereof, comprises the six CDRs of antibody ABL-CD3-36A. The anti-CD3 antibody, or antigen-binding fragment thereof, may comprise an LCDR1, an LCDR2, and an LCDR3 comprising the amino acid sequences of SEQ ID NOs: 36, 149, and 262, respectively; and an HCDR1, an HCDR2, and an HCDR3 comprising the amino acid sequences of SEQ ID NOs: 380, 571, and 609, respectively. In some embodiments, the anti-CD3 antibody, or antigen-binding fragment thereof, comprises the VL of antibody ABL-CD3-36. The VL may comprise the amino acid sequence of SEQ ID NO: 723. In some embodiments, the anti-CD3 antibody, or antigen-binding fragment thereof, comprises the VH of antibody ABL-CD3-36. The VH may comprise the amino acid sequence of SEQ ID NO: 950. In some embodiments, the anti-CD3 antibody, or antigen-binding fragment thereof, comprises the VL and VH of antibody ABL-CD3-36. In some embodiments, the anti-CD3 antibody, or antigen-binding fragment thereof, comprises a VL comprising the amino acid sequence of SEQ ID NO: 723 and a VH comprising the amino acid sequence of SEQ ID NO: 950. In some embodiments, the anti-CD3 antibody, or antigen-binding fragment thereof, comprises the VL of antibody ABL-CD3-36A. The VL may comprise the amino acid sequence of SEQ ID NO: 836. In some embodiments, the anti-CD3 antibody, or antigen-binding fragment thereof, comprises the VH of antibody ABL-CD3-36A. The VH may comprise the amino acid sequence of SEQ ID NO: 1063. In some embodiments, the anti-CD3 antibody, or antigen-binding fragment thereof, comprises the VL and VH of antibody ABL-CD3-36A. In some embodiments, the anti-CD3 antibody, or antigen-binding fragment thereof, comprises a VL comprising the amino acid sequence of SEQ ID NO: 836 and a VH comprising the amino acid sequence of SEQ ID NO: 1063. In some embodiments, the anti-CD3 antibody, or antigen-binding fragment thereof, comprises ABL-CD3-36_scFv_VH-VL. In some embodiments, the anti-CD3 antibody, or antigen-binding fragment thereof, comprises, from N-terminus to C-terminus, a VH comprising the amino acid sequence of SEQ ID NO: 950; a linker comprising the amino acid sequence of (Gly-Gly-Gly-Gly-Ser)n (SEQ ID NO: 1146), wherein n is 4; and a VL comprising the amino acid sequence of SEQ ID NO: 723. In some embodiments, the anti-CD3 antibody, or antigen-binding fragment thereof, comprises ABL-CD3-36_scFv_VL-VH. In some embodiments, the anti-CD3 antibody, or antigen-binding fragment thereof, comprises, from N-terminus to C-terminus, a VL comprising the amino acid sequence of SEQ ID NO: 723; a linker comprising the amino acid sequence of (Gly-Gly-Gly-Gly-Ser)n (SEQ ID NO: 1146), wherein n is 4; and a VH comprising the amino acid sequence of SEQ ID NO: 950. In some embodiments, the anti-CD3 antibody, or antigen-binding fragment thereof, comprises ABL-CD3-36A_scFv_VH-VL. In some embodiments, the anti-CD3 antibody, or antigen-binding fragment thereof, comprises the amino acid sequence of SEQ ID NO: 1163. In some embodiments, the anti-CD3 antibody, or antigen-binding fragment thereof, comprises ABL-CD3-36A_scFv_VL-VH. In some embodiments, the anti-CD3 antibody, or antigen-binding fragment thereof, comprises the amino acid sequence of SEQ ID NO: 1204.

In some embodiments, the anti-CD3 antibody, or antigen-binding fragment thereof, comprises the six CDRs of antibody ABL-CD3-37 and ABL-CD3-37A. The anti-CD3 antibody, or antigen-binding fragment thereof, may comprise an LCDR1, an LCDR2, and an LCDR3 comprising the amino acid sequences of SEQ ID NOs: 37, 150, and 263, respectively; and an HCDR1, an HCDR2, and an HCDR3 comprising the amino acid sequences of SEQ ID NOs: 381, 494, and 610, respectively. In some embodiments, the anti-CD3 antibody, or antigen-binding fragment thereof, comprises the VL of antibody ABL-CD3-37. The VL may comprise the amino acid sequence of SEQ ID NO: 724. In some embodiments, the anti-CD3 antibody, or antigen-binding fragment thereof, comprises the VH of antibody ABL-CD3-37. The VH may comprise the amino acid sequence of SEQ ID NO: 951. In some embodiments, the anti-CD3 antibody, or antigen-binding fragment thereof, comprises the VL and VH of antibody ABL-CD3-37. In some embodiments, the anti-CD3 antibody, or antigen-binding fragment thereof, comprises a VL comprising the amino acid sequence of SEQ ID NO: 724 and a VH comprising the amino acid sequence of SEQ ID NO: 951. In some embodiments, the anti-CD3 antibody, or antigen-binding fragment thereof, comprises the VL of antibody ABL-CD3-37A. The VL may comprise the amino acid sequence of SEQ ID NO: 837. In some embodiments, the anti-CD3 antibody, or antigen-binding fragment thereof, comprises the VH of antibody ABL-CD3-37A. The VH may comprise the amino acid sequence of SEQ ID NO: 1064. In some embodiments, the anti-CD3 antibody, or antigen-binding fragment thereof, comprises the VL and VH of antibody ABL-CD3-37A. In some embodiments, the anti-CD3 antibody, or antigen-binding fragment thereof, comprises a VL comprising the amino acid sequence of SEQ ID NO: 837 and a VH comprising the amino acid sequence of SEQ ID NO: 1064. In some embodiments, the anti-CD3 antibody, or antigen-binding fragment thereof, comprises ABL-CD3-37_scFv_VH-VL. In some embodiments, the anti-CD3 antibody, or antigen-binding fragment thereof, comprises, from N-terminus to C-terminus, a VH comprising the amino acid sequence of SEQ ID NO: 951; a linker comprising the amino acid sequence of (Gly-Gly-Gly-Gly-Ser)n (SEQ ID NO: 1146), wherein n is 4; and a VL comprising the amino acid sequence of SEQ ID NO: 724. In some embodiments, the anti-CD3 antibody, or antigen-binding fragment thereof, comprises ABL-CD3-37_scFv_VL-VH. In some embodiments, the anti-CD3 antibody, or antigen-binding fragment thereof, comprises, from N-terminus to C-terminus, a VL comprising the amino acid sequence of SEQ ID NO: 724; a linker comprising the amino acid sequence of (Gly-Gly-Gly-Gly-Ser)n (SEQ ID NO: 1146), wherein n is 4; and a VH comprising the amino acid sequence of SEQ ID NO: 951. In some embodiments, the anti-CD3 antibody, or antigen-binding fragment thereof, comprises ABL-CD3-37A_scFv_VH-VL. In some embodiments, the anti-CD3 antibody, or antigen-binding fragment thereof, comprises the amino acid sequence of SEQ ID NO: 1164. In some embodiments, the anti-CD3 antibody, or antigen-binding fragment thereof, comprises ABL-CD3-37A_scFv_VL-VH. In some embodiments, the anti-CD3 antibody, or antigen-binding fragment thereof, comprises the amino acid sequence of SEQ ID NO: 1205.

In some embodiments, the anti-CD3 antibody, or antigen-binding fragment thereof, comprises the six CDRs of antibody ABL-CD3-38 and ABL-CD3-38A. The anti-CD3 antibody, or antigen-binding fragment thereof, may comprise an LCDR1, an LCDR2, and an LCDR3 comprising the amino acid sequences of SEQ ID NOs: 38, 151, and 264, respectively; and an HCDR1, an HCDR2, and an HCDR3 comprising the amino acid sequences of SEQ ID NOs: 382, 495, and 611, respectively. In some embodiments, the anti-CD3 antibody, or antigen-binding fragment thereof, comprises the VL of antibody ABL-CD3-38. The VL may comprise the amino acid sequence of SEQ ID NO: 725. In some embodiments, the anti-CD3 antibody, or antigen-binding fragment thereof, comprises the VH of antibody ABL-CD3-38. The VH may comprise the amino acid sequence of SEQ ID NO: 952. In some embodiments, the anti-CD3 antibody, or antigen-binding fragment thereof, comprises the VL and VH of antibody ABL-CD3-38. In some embodiments, the anti-CD3 antibody, or antigen-binding fragment thereof, comprises a VL comprising the amino acid sequence of SEQ ID NO: 725 and a VH comprising the amino acid sequence of SEQ ID NO: 952. In some embodiments, the anti-CD3 antibody, or antigen-binding fragment thereof, comprises the VL of antibody ABL-CD3-38A. The VL may comprise the amino acid sequence of SEQ ID NO: 838. In some embodiments, the anti-CD3 antibody, or antigen-binding fragment thereof, comprises the VH of antibody ABL-CD3-38A. The VH may comprise the amino acid sequence of SEQ ID NO: 1065. In some embodiments, the anti-CD3 antibody, or antigen-binding fragment thereof, comprises the VL and VH of antibody ABL-CD3-38A. In some embodiments, the anti-CD3 antibody, or antigen-binding fragment thereof, comprises a VL comprising the amino acid sequence of SEQ ID NO: 838 and a VH comprising the amino acid sequence of SEQ ID NO: 1065. In some embodiments, the anti-CD3 antibody, or antigen-binding fragment thereof, comprises ABL-CD3-38_scFv_VH-VL. In some embodiments, the anti-CD3 antibody, or antigen-binding fragment thereof, comprises, from N-terminus to C-terminus, a VH comprising the amino acid sequence of SEQ ID NO: 952; a linker comprising the amino acid sequence of (Gly-Gly-Gly-Gly-Ser)n (SEQ ID NO: 1146), wherein n is 4; and a VL comprising the amino acid sequence of SEQ ID NO: 725. In some embodiments, the anti-CD3 antibody, or antigen-binding fragment thereof, comprises ABL-CD3-38_scFv_VL-VH. In some embodiments, the anti-CD3 antibody, or antigen-binding fragment thereof, comprises, from N-terminus to C-terminus, a VL comprising the amino acid sequence of SEQ ID NO: 725; a linker comprising the amino acid sequence of (Gly-Gly-Gly-Gly-Ser)n (SEQ ID NO: 1146), wherein n is 4; and a VH comprising the amino acid sequence of SEQ ID NO: 952. In some embodiments, the anti-CD3 antibody, or antigen-binding fragment thereof, comprises ABL-CD3-38A_scFv_VH-VL. In some embodiments, the anti-CD3 antibody, or antigen-binding fragment thereof, comprises, from N-terminus to C-terminus, a VH comprising the amino acid sequence of SEQ ID NO: 1065; a linker comprising the amino acid sequence of (Gly-Gly-Gly-Gly-Ser)n (SEQ ID NO: 1146), wherein n is 4; and a VL comprising the amino acid sequence of SEQ ID NO: 838. In some embodiments, the anti-CD3 antibody, or antigen-binding fragment thereof, comprises ABL-CD3-38A_scFv_VL-VH. In some embodiments, the anti-CD3 antibody, or antigen-binding fragment thereof, comprises, from N-terminus to C-terminus, a VL comprising the amino acid sequence of SEQ ID NO: 838; a linker comprising the amino acid sequence of (Gly-Gly-Gly-Gly-Ser)n (SEQ ID NO: 1146), wherein n is 4; and a VH comprising the amino acid sequence of SEQ ID NO: 1065.

In some embodiments, the anti-CD3 antibody, or antigen-binding fragment thereof, comprises the six CDRs of antibody ABL-CD3-39 and ABL-CD3-39A. The anti-CD3 antibody, or antigen-binding fragment thereof, may comprise an LCDR1, an LCDR2, and an LCDR3 comprising the amino acid sequences of SEQ ID NOs: 39, 152, and 265, respectively; and an HCDR1, an HCDR2, and an HCDR3 comprising the amino acid sequences of SEQ ID NOs: 383, 496, and 612, respectively. In some embodiments, the anti-CD3 antibody, or antigen-binding fragment thereof, comprises the VL of antibody ABL-CD3-39. The VL may comprise the amino acid sequence of SEQ ID NO: 726. In some embodiments, the anti-CD3 antibody, or antigen-binding fragment thereof, comprises the VH of antibody ABL-CD3-39. The VH may comprise the amino acid sequence of SEQ ID NO: 953. In some embodiments, the anti-CD3 antibody, or antigen-binding fragment thereof, comprises the VL and VH of antibody ABL-CD3-39. In some embodiments, the anti-CD3 antibody, or antigen-binding fragment thereof, comprises a VL comprising the amino acid sequence of SEQ ID NO: 726 and a VH comprising the amino acid sequence of SEQ ID NO: 953. In some embodiments, the anti-CD3 antibody, or antigen-binding fragment thereof, comprises the VL of antibody ABL-CD3-39A. The VL may comprise the amino acid sequence of SEQ ID NO: 839. In some embodiments, the anti-CD3 antibody, or antigen-binding fragment thereof, comprises the VH of antibody ABL-CD3-39A. The VH may comprise the amino acid sequence of SEQ ID NO: 1066. In some embodiments, the anti-CD3 antibody, or antigen-binding fragment thereof, comprises the VL and VH of antibody ABL-CD3-39A. In some embodiments, the anti-CD3 antibody, or antigen-binding fragment thereof, comprises a VL comprising the amino acid sequence of SEQ ID NO: 839 and a VH comprising the amino acid sequence of SEQ ID NO: 1066. In some embodiments, the anti-CD3 antibody, or antigen-binding fragment thereof, comprises ABL-CD3-39_scFv_VH-VL. In some embodiments, the anti-CD3 antibody, or antigen-binding fragment thereof, comprises, from N-terminus to C-terminus, a VH comprising the amino acid sequence of SEQ ID NO: 953; a linker comprising the amino acid sequence of (Gly-Gly-Gly-Gly-Ser)n (SEQ ID NO: 1146), wherein n is 4; and a VL comprising the amino acid sequence of SEQ ID NO: 726. In some embodiments, the anti-CD3 antibody, or antigen-binding fragment thereof, comprises ABL-CD3-39_scFv_VL-VH. In some embodiments, the anti-CD3 antibody, or antigen-binding fragment thereof, comprises, from N-terminus to C-terminus, a VL comprising the amino acid sequence of SEQ ID NO: 726; a linker comprising the amino acid sequence of (Gly-Gly-Gly-Gly-Ser)n (SEQ ID NO: 1146), wherein n is 4; and a VH comprising the amino acid sequence of SEQ ID NO: 953. In some embodiments, the anti-CD3 antibody, or antigen-binding fragment thereof, comprises ABL-CD3-39A_scFv_VH-VL. In some embodiments, the anti-CD3 antibody, or antigen-binding fragment thereof, comprises the amino acid sequence of SEQ ID NO: 1165. In some embodiments, the anti-CD3 antibody, or antigen-binding fragment thereof, comprises ABL-CD3-39A_scFv_VL-VH. In some embodiments, the anti-CD3 antibody, or antigen-binding fragment thereof, comprises the amino acid sequence of SEQ ID NO: 1206.

In some embodiments, the anti-CD3 antibody, or antigen-binding fragment thereof, comprises the six CDRs of antibody ABL-CD3-40 and ABL-CD3-40A. The anti-CD3 antibody, or antigen-binding fragment thereof, may comprise an LCDR1, an LCDR2, and an LCDR3 comprising the amino acid sequences of SEQ ID NOs: 40, 153, and 266, respectively; and an HCDR1, an HCDR2, and an HCDR3 comprising the amino acid sequences of SEQ ID NOs: 384, 497, and 613, respectively. In some embodiments, the anti-CD3 antibody, or antigen-binding fragment thereof, comprises the VL of antibody ABL-CD3-40. The VL may comprise the amino acid sequence of SEQ ID NO: 727. In some embodiments, the anti-CD3 antibody, or antigen-binding fragment thereof, comprises the VH of antibody ABL-CD3-40. The VH may comprise the amino acid sequence of SEQ ID NO: 954. In some embodiments, the anti-CD3 antibody, or antigen-binding fragment thereof, comprises the VL and VH of antibody ABL-CD3-40. In some embodiments, the anti-CD3 antibody, or antigen-binding fragment thereof, comprises a VL comprising the amino acid sequence of SEQ ID NO: 727 and a VH comprising the amino acid sequence of SEQ ID NO: 954. In some embodiments, the anti-CD3 antibody, or antigen-binding fragment thereof, comprises the VL of antibody ABL-CD3-40A. The VL may comprise the amino acid sequence of SEQ ID NO: 840. In some embodiments, the anti-CD3 antibody, or antigen-binding fragment thereof, comprises the VH of antibody ABL-CD3-40A. The VH may comprise the amino acid sequence of SEQ ID NO: 1067. In some embodiments, the anti-CD3 antibody, or antigen-binding fragment thereof, comprises the VL and VH of antibody ABL-CD3-40A. In some embodiments, the anti-CD3 antibody, or antigen-binding fragment thereof, comprises a VL comprising the amino acid sequence of SEQ ID NO: 840 and a VH comprising the amino acid sequence of SEQ ID NO: 1067. In some embodiments, the anti-CD3 antibody, or antigen-binding fragment thereof, comprises ABL-CD3-40_scFv_VH-VL. In some embodiments, the anti-CD3 antibody, or antigen-binding fragment thereof, comprises, from N-terminus to C-terminus, a VH comprising the amino acid sequence of SEQ ID NO: 954; a linker comprising the amino acid sequence of (Gly-Gly-Gly-Gly-Ser)n (SEQ ID NO: 1146), wherein n is 4; and a VL comprising the amino acid sequence of SEQ ID NO: 727. In some embodiments, the anti-CD3 antibody, or antigen-binding fragment thereof, comprises ABL-CD3-40_scFv_VL-VH. In some embodiments, the anti-CD3 antibody, or antigen-binding fragment thereof, comprises, from N-terminus to C-terminus, a VL comprising the amino acid sequence of SEQ ID NO: 727; a linker comprising the amino acid sequence of (Gly-Gly-Gly-Gly-Ser)n (SEQ ID NO: 1146), wherein n is 4; and a VH comprising the amino acid sequence of SEQ ID NO: 954. In some embodiments, the anti-CD3 antibody, or antigen-binding fragment thereof, comprises ABL-CD3-40A_scFv_VH-VL. In some embodiments, the anti-CD3 antibody, or antigen-binding fragment thereof, comprises the amino acid sequence of SEQ ID NO: 1166. In some embodiments, the anti-CD3 antibody, or antigen-binding fragment thereof, comprises ABL-CD3-40A_scFv_VL-VH. In some embodiments, the anti-CD3 antibody, or antigen-binding fragment thereof, comprises the amino acid sequence of SEQ ID NO: 1207.

In some embodiments, the anti-CD3 antibody, or antigen-binding fragment thereof, comprises the six CDRs of antibody ABL-CD3-41. The anti-CD3 antibody, or antigen-binding fragment thereof, may comprise an LCDR1, an LCDR2, and an LCDR3 comprising the amino acid sequences of SEQ ID NOs: 41, 154, and 267, respectively; and an HCDR1, an HCDR2, and an HCDR3 comprising the amino acid sequences of SEQ ID NOs: 385, 498, and 614, respectively. In some embodiments, the anti-CD3 antibody, or antigen-binding fragment thereof, comprises the six CDRs of antibody ABL-CD3-41A. The anti-CD3 antibody, or antigen-binding fragment thereof, may comprise an LCDR1, an LCDR2, and an LCDR3 comprising the amino acid sequences of SEQ ID NOs: 41, 154, and 340, respectively; and an HCDR1, an HCDR2, and an HCDR3 comprising the amino acid sequences of SEQ ID NOs: 385, 498, and 614, respectively. In some embodiments, the anti-CD3 antibody, or antigen-binding fragment thereof, comprises the VL of antibody ABL-CD3-41. The VL may comprise the amino acid sequence of SEQ ID NO: 728. In some embodiments, the anti-CD3 antibody, or antigen-binding fragment thereof, comprises the VH of antibody ABL-CD3-41. The VH may comprise the amino acid sequence of SEQ ID NO: 955. In some embodiments, the anti-CD3 antibody, or antigen-binding fragment thereof, comprises the VL and VH of antibody ABL-CD3-41. In some embodiments, the anti-CD3 antibody, or antigen-binding fragment thereof, comprises a VL comprising the amino acid sequence of SEQ ID NO: 728 and a VH comprising the amino acid sequence of SEQ ID NO: 955. In some embodiments, the anti-CD3 antibody, or antigen-binding fragment thereof, comprises the VL of antibody ABL-CD3-41A. The VL may comprise the amino acid sequence of SEQ ID NO: 841. In some embodiments, the anti-CD3 antibody, or antigen-binding fragment thereof, comprises the VH of antibody ABL-CD3-41A. The VH may comprise the amino acid sequence of SEQ ID NO: 1068. In some embodiments, the anti-CD3 antibody, or antigen-binding fragment thereof, comprises the VL and VH of antibody ABL-CD3-41A. In some embodiments, the anti-CD3 antibody, or antigen-binding fragment thereof, comprises a VL comprising the amino acid sequence of SEQ ID NO: 841 and a VH comprising the amino acid sequence of SEQ ID NO: 1068. In some embodiments, the anti-CD3 antibody, or antigen-binding fragment thereof, comprises ABL-CD3-41_scFv_VH-VL. In some embodiments, the anti-CD3 antibody, or antigen-binding fragment thereof, comprises, from N-terminus to C-terminus, a VH comprising the amino acid sequence of SEQ ID NO: 955; a linker comprising the amino acid sequence of (Gly-Gly-Gly-Gly-Ser)n (SEQ ID NO: 1146), wherein n is 4; and a VL comprising the amino acid sequence of SEQ ID NO: 728. In some embodiments, the anti-CD3 antibody, or antigen-binding fragment thereof, comprises ABL-CD3-41_scFv_VL-VH. In some embodiments, the anti-CD3 antibody, or antigen-binding fragment thereof, comprises, from N-terminus to C-terminus, a VL comprising the amino acid sequence of SEQ ID NO: 728; a linker comprising the amino acid sequence of (Gly-Gly-Gly-Gly-Ser)n (SEQ ID NO: 1146), wherein n is 4; and a VH comprising the amino acid sequence of SEQ ID NO: 955. In some embodiments, the anti-CD3 antibody, or antigen-binding fragment thereof, comprises ABL-CD3-41A_scFv_VH-VL. In some embodiments, the anti-CD3 antibody, or antigen-binding fragment thereof, comprises the amino acid sequence of SEQ ID NO: 1167. In some embodiments, the anti-CD3 antibody, or antigen-binding fragment thereof, comprises ABL-CD3-41A_scFv_VL-VH. In some embodiments, the anti-CD3 antibody, or antigen-binding fragment thereof, comprises the amino acid sequence of SEQ ID NO: 1208.

In some embodiments, the anti-CD3 antibody, or antigen-binding fragment thereof, comprises the six CDRs of antibody ABL-CD3-42 and ABL-CD3-42A. The anti-CD3 antibody, or antigen-binding fragment thereof, may comprise an LCDR1, an LCDR2, and an LCDR3 comprising the amino acid sequences of SEQ ID NOs: 42, 155, and 268, respectively; and an HCDR1, an HCDR2, and an HCDR3 comprising the amino acid sequences of SEQ ID NOs: 386, 499, and 615, respectively. In some embodiments, the anti-CD3 antibody, or antigen-binding fragment thereof, comprises the VL of antibody ABL-CD3-42. The VL may comprise the amino acid sequence of SEQ ID NO: 729. In some embodiments, the anti-CD3 antibody, or antigen-binding fragment thereof, comprises the VH of antibody ABL-CD3-42. The VH may comprise the amino acid sequence of SEQ ID NO: 956. In some embodiments, the anti-CD3 antibody, or antigen-binding fragment thereof, comprises the VL and VH of antibody ABL-CD3-42. In some embodiments, the anti-CD3 antibody, or antigen-binding fragment thereof, comprises a VL comprising the amino acid sequence of SEQ ID NO: 729 and a VH comprising the amino acid sequence of SEQ ID NO: 956. In some embodiments, the anti-CD3 antibody, or antigen-binding fragment thereof, comprises the VL of antibody ABL-CD3-42A. The VL may comprise the amino acid sequence of SEQ ID NO: 842. In some embodiments, the anti-CD3 antibody, or antigen-binding fragment thereof, comprises the VH of antibody ABL-CD3-42A. The VH may comprise the amino acid sequence of SEQ ID NO: 1069. In some embodiments, the anti-CD3 antibody, or antigen-binding fragment thereof, comprises the VL and VH of antibody ABL-CD3-42A. In some embodiments, the anti-CD3 antibody, or antigen-binding fragment thereof, comprises a VL comprising the amino acid sequence of SEQ ID NO: 842 and a VH comprising the amino acid sequence of SEQ ID NO: 1069. In some embodiments, the anti-CD3 antibody, or antigen-binding fragment thereof, comprises ABL-CD3-42_scFv_VH-VL. In some embodiments, the anti-CD3 antibody, or antigen-binding fragment thereof, comprises, from N-terminus to C-terminus, a VH comprising the amino acid sequence of SEQ ID NO: 956; a linker comprising the amino acid sequence of (Gly-Gly-Gly-Gly-Ser)n (SEQ ID NO: 1146), wherein n is 4; and a VL comprising the amino acid sequence of SEQ ID NO: 729. In some embodiments, the anti-CD3 antibody, or antigen-binding fragment thereof, comprises ABL-CD3-42_scFv_VL-VH. In some embodiments, the anti-CD3 antibody, or antigen-binding fragment thereof, comprises, from N-terminus to C-terminus, a VL comprising the amino acid sequence of SEQ ID NO: 729; a linker comprising the amino acid sequence of (Gly-Gly-Gly-Gly-Ser)n (SEQ ID NO: 1146), wherein n is 4; and a VH comprising the amino acid sequence of SEQ ID NO: 956. In some embodiments, the anti-CD3 antibody, or antigen-binding fragment thereof, comprises ABL-CD3-42A_scFv_VH-VL. In some embodiments, the anti-CD3 antibody, or antigen-binding fragment thereof, comprises, from N-terminus to C-terminus, a VH comprising the amino acid sequence of SEQ ID NO: 1069; a linker comprising the amino acid sequence of (Gly-Gly-Gly-Gly-Ser)n (SEQ ID NO: 1146), wherein n is 4; and a VL comprising the amino acid sequence of SEQ ID NO: 842. In some embodiments, the anti-CD3 antibody, or antigen-binding fragment thereof, comprises ABL-CD3-42A_scFv_VL-VH. In some embodiments, the anti-CD3 antibody, or antigen-binding fragment thereof, comprises, from N-terminus to C-terminus, a VL comprising the amino acid sequence of SEQ ID NO: 842; a linker comprising the amino acid sequence of (Gly-Gly-Gly-Gly-Ser)n (SEQ ID NO: 1146), wherein n is 4; and a VH comprising the amino acid sequence of SEQ ID NO: 1069.

In some embodiments, the anti-CD3 antibody, or antigen-binding fragment thereof, comprises the six CDRs of antibody ABL-CD3-43. The anti-CD3 antibody, or antigen-binding fragment thereof, may comprise an LCDR1, an LCDR2, and an LCDR3 comprising the amino acid sequences of SEQ ID NOs: 43, 156, and 269, respectively; and an HCDR1, an HCDR2, and an HCDR3 comprising the amino acid sequences of SEQ ID NOs: 387, 500, and 616, respectively. In some embodiments, the anti-CD3 antibody, or antigen-binding fragment thereof, comprises the six CDRs of antibody ABL-CD3-43A. The anti-CD3 antibody, or antigen-binding fragment thereof, may comprise an LCDR1, an LCDR2, and an LCDR3 comprising the amino acid sequences of SEQ ID NOs: 43, 156, and 341, respectively; and an HCDR1, an HCDR2, and an HCDR3 comprising the amino acid sequences of SEQ ID NOs: 387, 500, and 616, respectively. In some embodiments, the anti-CD3 antibody, or antigen-binding fragment thereof, comprises the VL of antibody ABL-CD3-43. The VL may comprise the amino acid sequence of SEQ ID NO: 730. In some embodiments, the anti-CD3 antibody, or antigen-binding fragment thereof, comprises the VH of antibody ABL-CD3-43. The VH may comprise the amino acid sequence of SEQ ID NO: 957. In some embodiments, the anti-CD3 antibody, or antigen-binding fragment thereof, comprises the VL and VH of antibody ABL-CD3-43. In some embodiments, the anti-CD3 antibody, or antigen-binding fragment thereof, comprises a VL comprising the amino acid sequence of SEQ ID NO: 730 and a VH comprising the amino acid sequence of SEQ ID NO: 957. In some embodiments, the anti-CD3 antibody, or antigen-binding fragment thereof, comprises the VL of antibody ABL-CD3-43A. The VL may comprise the amino acid sequence of SEQ ID NO: 843. In some embodiments, the anti-CD3 antibody, or antigen-binding fragment thereof, comprises the VH of antibody ABL-CD3-43A. The VH may comprise the amino acid sequence of SEQ ID NO: 1070. In some embodiments, the anti-CD3 antibody, or antigen-binding fragment thereof, comprises the VL and VH of antibody ABL-CD3-43A. In some embodiments, the anti-CD3 antibody, or antigen-binding fragment thereof, comprises a VL comprising the amino acid sequence of SEQ ID NO: 843 and a VH comprising the amino acid sequence of SEQ ID NO: 1070. In some embodiments, the anti-CD3 antibody, or antigen-binding fragment thereof, comprises ABL-CD3-43_scFv_VH-VL. In some embodiments, the anti-CD3 antibody, or antigen-binding fragment thereof, comprises, from N-terminus to C-terminus, a VH comprising the amino acid sequence of SEQ ID NO: 957; a linker comprising the amino acid sequence of (Gly-Gly-Gly-Gly-Ser)n (SEQ ID NO: 1146), wherein n is 4; and a VL comprising the amino acid sequence of SEQ ID NO: 730. In some embodiments, the anti-CD3 antibody, or antigen-binding fragment thereof, comprises ABL-CD3-43_scFv_VL-VH. In some embodiments, the anti-CD3 antibody, or antigen-binding fragment thereof, comprises, from N-terminus to C-terminus, a VL comprising the amino acid sequence of SEQ ID NO: 730; a linker comprising the amino acid sequence of (Gly-Gly-Gly-Gly-Ser)n (SEQ ID NO: 1146), wherein n is 4; and a VH comprising the amino acid sequence of SEQ ID NO: 957. In some embodiments, the anti-CD3 antibody, or antigen-binding fragment thereof, comprises ABL-CD3-43A_scFv_VH-VL. In some embodiments, the anti-CD3 antibody, or antigen-binding fragment thereof, comprises the amino acid sequence of SEQ ID NO: 1168. In some embodiments, the anti-CD3 antibody, or antigen-binding fragment thereof, comprises ABL-CD3-43A_scFv_VL-VH. In some embodiments, the anti-CD3 antibody, or antigen-binding fragment thereof, comprises the amino acid sequence of SEQ ID NO: 1209.

In some embodiments, the anti-CD3 antibody, or antigen-binding fragment thereof, comprises the six CDRs of antibody ABL-CD3-44 and ABL-CD3-44A. The anti-CD3 antibody, or antigen-binding fragment thereof, may comprise an LCDR1, an LCDR2, and an LCDR3 comprising the amino acid sequences of SEQ ID NOs: 44, 157, and 270, respectively; and an HCDR1, an HCDR2, and an HCDR3 comprising the amino acid sequences of SEQ ID NOs: 388, 501, and 617, respectively. In some embodiments, the anti-CD3 antibody, or antigen-binding fragment thereof, comprises the VL of antibody ABL-CD3-44. The VL may comprise the amino acid sequence of SEQ ID NO: 731. In some embodiments, the anti-CD3 antibody, or antigen-binding fragment thereof, comprises the VH of antibody ABL-CD3-44. The VH may comprise the amino acid sequence of SEQ ID NO: 958. In some embodiments, the anti-CD3 antibody, or antigen-binding fragment thereof, comprises the VL and VH of antibody ABL-CD3-44. In some embodiments, the anti-CD3 antibody, or antigen-binding fragment thereof, comprises a VL comprising the amino acid sequence of SEQ ID NO: 731 and a VH comprising the amino acid sequence of SEQ ID NO: 958. In some embodiments, the anti-CD3 antibody, or antigen-binding fragment thereof, comprises the VL of antibody ABL-CD3-44A. The VL may comprise the amino acid sequence of SEQ ID NO: 844. In some embodiments, the anti-CD3 antibody, or antigen-binding fragment thereof, comprises the VH of antibody ABL-CD3-44A. The VH may comprise the amino acid sequence of SEQ ID NO: 1071. In some embodiments, the anti-CD3 antibody, or antigen-binding fragment thereof, comprises the VL and VH of antibody ABL-CD3-44A. In some embodiments, the anti-CD3 antibody, or antigen-binding fragment thereof, comprises a VL comprising the amino acid sequence of SEQ ID NO: 844 and a VH comprising the amino acid sequence of SEQ ID NO: 1071. In some embodiments, the anti-CD3 antibody, or antigen-binding fragment thereof, comprises ABL-CD3-44_scFv_VH-VL. In some embodiments, the anti-CD3 antibody, or antigen-binding fragment thereof, comprises, from N-terminus to C-terminus, a VH comprising the amino acid sequence of SEQ ID NO: 958; a linker comprising the amino acid sequence of (Gly-Gly-Gly-Gly-Ser)n (SEQ ID NO: 1146), wherein n is 4; and a VL comprising the amino acid sequence of SEQ ID NO: 731. In some embodiments, the anti-CD3 antibody, or antigen-binding fragment thereof, comprises ABL-CD3-44_scFv_VL-VH. In some embodiments, the anti-CD3 antibody, or antigen-binding fragment thereof, comprises, from N-terminus to C-terminus, a VL comprising the amino acid sequence of SEQ ID NO: 731; a linker comprising the amino acid sequence of (Gly-Gly-Gly-Gly-Ser)n (SEQ ID NO: 1146), wherein n is 4; and a VH comprising the amino acid sequence of SEQ ID NO: 958. In some embodiments, the anti-CD3 antibody, or antigen-binding fragment thereof, comprises ABL-CD3-44A_scFv_VH-VL. In some embodiments, the anti-CD3 antibody, or antigen-binding fragment thereof, comprises the amino acid sequence of SEQ ID NO: 1169. In some embodiments, the anti-CD3 antibody, or antigen-binding fragment thereof, comprises ABL-CD3-44A_scFv_VL-VH. In some embodiments, the anti-CD3 antibody, or antigen-binding fragment thereof, comprises the amino acid sequence of SEQ ID NO: 1210.

In some embodiments, the anti-CD3 antibody, or antigen-binding fragment thereof, comprises the six CDRs of antibody ABL-CD3-45 and ABL-CD3-45A. The anti-CD3 antibody, or antigen-binding fragment thereof, may comprise an LCDR1, an LCDR2, and an LCDR3 comprising the amino acid sequences of SEQ ID NOs: 45, 158, and 271, respectively; and an HCDR1, an HCDR2, and an HCDR3 comprising the amino acid sequences of SEQ ID NOs: 389, 502, and 618, respectively. In some embodiments, the anti-CD3 antibody, or antigen-binding fragment thereof, comprises the VL of antibody ABL-CD3-45. The VL may comprise the amino acid sequence of SEQ ID NO: 732. In some embodiments, the anti-CD3 antibody, or antigen-binding fragment thereof, comprises the VH of antibody ABL-CD3-45. The VH may comprise the amino acid sequence of SEQ ID NO: 959. In some embodiments, the anti-CD3 antibody, or antigen-binding fragment thereof, comprises the VL and VH of antibody ABL-CD3-45. In some embodiments, the anti-CD3 antibody, or antigen-binding fragment thereof, comprises a VL comprising the amino acid sequence of SEQ ID NO: 732 and a VH comprising the amino acid sequence of SEQ ID NO: 959. In some embodiments, the anti-CD3 antibody, or antigen-binding fragment thereof, comprises the VL of antibody ABL-CD3-45A. The VL may comprise the amino acid sequence of SEQ ID NO: 845. In some embodiments, the anti-CD3 antibody, or antigen-binding fragment thereof, comprises the VH of antibody ABL-CD3-45A. The VH may comprise the amino acid sequence of SEQ ID NO: 1072. In some embodiments, the anti-CD3 antibody, or antigen-binding fragment thereof, comprises the VL and VH of antibody ABL-CD3-45A. In some embodiments, the anti-CD3 antibody, or antigen-binding fragment thereof, comprises a VL comprising the amino acid sequence of SEQ ID NO: 845 and a VH comprising the amino acid sequence of SEQ ID NO: 1072. In some embodiments, the anti-CD3 antibody, or antigen-binding fragment thereof, comprises ABL-CD3-45_scFv_VH-VL. In some embodiments, the anti-CD3 antibody, or antigen-binding fragment thereof, comprises, from N-terminus to C-terminus, a VH comprising the amino acid sequence of SEQ ID NO: 959; a linker comprising the amino acid sequence of (Gly-Gly-Gly-Gly-Ser)n (SEQ ID NO: 1146), wherein n is 4; and a VL comprising the amino acid sequence of SEQ ID NO: 732. In some embodiments, the anti-CD3 antibody, or antigen-binding fragment thereof, comprises ABL-CD3-45_scFv_VL-VH. In some embodiments, the anti-CD3 antibody, or antigen-binding fragment thereof, comprises, from N-terminus to C-terminus, a VL comprising the amino acid sequence of SEQ ID NO: 732; a linker comprising the amino acid sequence of (Gly-Gly-Gly-Gly-Ser)n (SEQ ID NO: 1146), wherein n is 4; and a VH comprising the amino acid sequence of SEQ ID NO: 959. In some embodiments, the anti-CD3 antibody, or antigen-binding fragment thereof, comprises ABL-CD3-45A_scFv_VH-VL. In some embodiments, the anti-CD3 antibody, or antigen-binding fragment thereof, comprises the amino acid sequence of SEQ ID NO: 1170. In some embodiments, the anti-CD3 antibody, or antigen-binding fragment thereof, comprises ABL-CD3-45A_scFv_VL-VH. In some embodiments, the anti-CD3 antibody, or antigen-binding fragment thereof, comprises the amino acid sequence of SEQ ID NO: 1211.

In some embodiments, the anti-CD3 antibody, or antigen-binding fragment thereof, comprises the six CDRs of antibody ABL-CD3-46 and ABL-CD3-46A. The anti-CD3 antibody, or antigen-binding fragment thereof, may comprise an LCDR1, an LCDR2, and an LCDR3 comprising the amino acid sequences of SEQ ID NOs: 46, 159, and 272, respectively; and an HCDR1, an HCDR2, and an HCDR3 comprising the amino acid sequences of SEQ ID NOs: 390, 503, and 619, respectively. In some embodiments, the anti-CD3 antibody, or antigen-binding fragment thereof, comprises the VL of antibody ABL-CD3-46. The VL may comprise the amino acid sequence of SEQ ID NO: 733. In some embodiments, the anti-CD3 antibody, or antigen-binding fragment thereof, comprises the VH of antibody ABL-CD3-46. The VH may comprise the amino acid sequence of SEQ ID NO: 960. In some embodiments, the anti-CD3 antibody, or antigen-binding fragment thereof, comprises the VL and VH of antibody ABL-CD3-46. In some embodiments, the anti-CD3 antibody, or antigen-binding fragment thereof, comprises a VL comprising the amino acid sequence of SEQ ID NO: 733 and a VH comprising the amino acid sequence of SEQ ID NO: 960. In some embodiments, the anti-CD3 antibody, or antigen-binding fragment thereof, comprises the VL of antibody ABL-CD3-46A. The VL may comprise the amino acid sequence of SEQ ID NO: 846. In some embodiments, the anti-CD3 antibody, or antigen-binding fragment thereof, comprises the VH of antibody ABL-CD3-46A. The VH may comprise the amino acid sequence of SEQ ID NO: 1073. In some embodiments, the anti-CD3 antibody, or antigen-binding fragment thereof, comprises the VL and VH of antibody ABL-CD3-46A. In some embodiments, the anti-CD3 antibody, or antigen-binding fragment thereof, comprises a VL comprising the amino acid sequence of SEQ ID NO: 846 and a VH comprising the amino acid sequence of SEQ ID NO: 1073. In some embodiments, the anti-CD3 antibody, or antigen-binding fragment thereof, comprises ABL-CD3-46_scFv_VH-VL. In some embodiments, the anti-CD3 antibody, or antigen-binding fragment thereof, comprises, from N-terminus to C-terminus, a VH comprising the amino acid sequence of SEQ ID NO: 960; a linker comprising the amino acid sequence of (Gly-Gly-Gly-Gly-Ser)n (SEQ ID NO: 1146), wherein n is 4; and a VL comprising the amino acid sequence of SEQ ID NO: 733. In some embodiments, the anti-CD3 antibody, or antigen-binding fragment thereof, comprises ABL-CD3-46_scFv_VL-VH. In some embodiments, the anti-CD3 antibody, or antigen-binding fragment thereof, comprises, from N-terminus to C-terminus, a VL comprising the amino acid sequence of SEQ ID NO: 733; a linker comprising the amino acid sequence of (Gly-Gly-Gly-Gly-Ser)n (SEQ ID NO: 1146), wherein n is 4; and a VH comprising the amino acid sequence of SEQ ID NO: 960. In some embodiments, the anti-CD3 antibody, or antigen-binding fragment thereof, comprises ABL-CD3-46A_scFv_VH-VL. In some embodiments, the anti-CD3 antibody, or antigen-binding fragment thereof, comprises, from N-terminus to C-terminus, a VH comprising the amino acid sequence of SEQ ID NO: 1073; a linker comprising the amino acid sequence of (Gly-Gly-Gly-Gly-Ser)n (SEQ ID NO: 1146), wherein n is 4; and a VL comprising the amino acid sequence of SEQ ID NO: 846. In some embodiments, the anti-CD3 antibody, or antigen-binding fragment thereof, comprises ABL-CD3-46A_scFv_VL-VH. In some embodiments, the anti-CD3 antibody, or antigen-binding fragment thereof, comprises, from N-terminus to C-terminus, a VL comprising the amino acid sequence of SEQ ID NO: 846; a linker comprising the amino acid sequence of (Gly-Gly-Gly-Gly-Ser)n (SEQ ID NO: 1146), wherein n is 4; and a VH comprising the amino acid sequence of SEQ ID NO: 1073.

In some embodiments, the anti-CD3 antibody, or antigen-binding fragment thereof, comprises the six CDRs of antibody ABL-CD3-47 and ABL-CD3-47A. The anti-CD3 antibody, or antigen-binding fragment thereof, may comprise an LCDR1, an LCDR2, and an LCDR3 comprising the amino acid sequences of SEQ ID NOs: 47, 160, and 273, respectively; and an HCDR1, an HCDR2, and an HCDR3 comprising the amino acid sequences of SEQ ID NOs: 391, 504, and 620, respectively. In some embodiments, the anti-CD3 antibody, or antigen-binding fragment thereof, comprises the VL of antibody ABL-CD3-47. The VL may comprise the amino acid sequence of SEQ ID NO: 734. In some embodiments, the anti-CD3 antibody, or antigen-binding fragment thereof, comprises the VH of antibody ABL-CD3-47. The VH may comprise the amino acid sequence of SEQ ID NO: 961. In some embodiments, the anti-CD3 antibody, or antigen-binding fragment thereof, comprises the VL and VH of antibody ABL-CD3-47. In some embodiments, the anti-CD3 antibody, or antigen-binding fragment thereof, comprises a VL comprising the amino acid sequence of SEQ ID NO: 734 and a VH comprising the amino acid sequence of SEQ ID NO: 961. In some embodiments, the anti-CD3 antibody, or antigen-binding fragment thereof, comprises the VL of antibody ABL-CD3-47A. The VL may comprise the amino acid sequence of SEQ ID NO: 847. In some embodiments, the anti-CD3 antibody, or antigen-binding fragment thereof, comprises the VH of antibody ABL-CD3-47A. The VH may comprise the amino acid sequence of SEQ ID NO: 1074. In some embodiments, the anti-CD3 antibody, or antigen-binding fragment thereof, comprises the VL and VH of antibody ABL-CD3-47A. In some embodiments, the anti-CD3 antibody, or antigen-binding fragment thereof, comprises a VL comprising the amino acid sequence of SEQ ID NO: 847 and a VH comprising the amino acid sequence of SEQ ID NO: 1074. In some embodiments, the anti-CD3 antibody, or antigen-binding fragment thereof, comprises ABL-CD3-47_scFv_VH-VL. In some embodiments, the anti-CD3 antibody, or antigen-binding fragment thereof, comprises, from N-terminus to C-terminus, a VH comprising the amino acid sequence of SEQ ID NO: 961; a linker comprising the amino acid sequence of (Gly-Gly-Gly-Gly-Ser)n (SEQ ID NO: 1146), wherein n is 4; and a VL comprising the amino acid sequence of SEQ ID NO: 734. In some embodiments, the anti-CD3 antibody, or antigen-binding fragment thereof, comprises ABL-CD3-47_scFv_VL-VH. In some embodiments, the anti-CD3 antibody, or antigen-binding fragment thereof, comprises, from N-terminus to C-terminus, a VL comprising the amino acid sequence of SEQ ID NO: 734; a linker comprising the amino acid sequence of (Gly-Gly-Gly-Gly-Ser)n (SEQ ID NO: 1146), wherein n is 4; and a VH comprising the amino acid sequence of SEQ ID NO: 961. In some embodiments, the anti-CD3 antibody, or antigen-binding fragment thereof, comprises ABL-CD3-47A_scFv_VH-VL. In some embodiments, the anti-CD3 antibody, or antigen-binding fragment thereof, comprises the amino acid sequence of SEQ ID NO: 1171. In some embodiments, the anti-CD3 antibody, or antigen-binding fragment thereof, comprises ABL-CD3-47A_scFv_VL-VH. In some embodiments, the anti-CD3 antibody, or antigen-binding fragment thereof, comprises the amino acid sequence of SEQ ID NO: 1212.

In some embodiments, the anti-CD3 antibody, or antigen-binding fragment thereof, comprises the six CDRs of antibody ABL-CD3-48 and ABL-CD3-48A. The anti-CD3 antibody, or antigen-binding fragment thereof, may comprise an LCDR1, an LCDR2, and an LCDR3 comprising the amino acid sequences of SEQ ID NOs: 48, 161, and 274, respectively; and an HCDR1, an HCDR2, and an HCDR3 comprising the amino acid sequences of SEQ ID NOs: 392, 505, and 621, respectively. In some embodiments, the anti-CD3 antibody, or antigen-binding fragment thereof, comprises the VL of antibody ABL-CD3-48. The VL may comprise the amino acid sequence of SEQ ID NO: 735. In some embodiments, the anti-CD3 antibody, or antigen-binding fragment thereof, comprises the VH of antibody ABL-CD3-48. The VH may comprise the amino acid sequence of SEQ ID NO: 962. In some embodiments, the anti-CD3 antibody, or antigen-binding fragment thereof, comprises the VL and VH of antibody ABL-CD3-48. In some embodiments, the anti-CD3 antibody, or antigen-binding fragment thereof, comprises a VL comprising the amino acid sequence of SEQ ID NO: 735 and a VH comprising the amino acid sequence of SEQ ID NO: 962. In some embodiments, the anti-CD3 antibody, or antigen-binding fragment thereof, comprises the VL of antibody ABL-CD3-48A. The VL may comprise the amino acid sequence of SEQ ID NO: 848. In some embodiments, the anti-CD3 antibody, or antigen-binding fragment thereof, comprises the VH of antibody ABL-CD3-48A. The VH may comprise the amino acid sequence of SEQ ID NO: 1075. In some embodiments, the anti-CD3 antibody, or antigen-binding fragment thereof, comprises the VL and VH of antibody ABL-CD3-48A. In some embodiments, the anti-CD3 antibody, or antigen-binding fragment thereof, comprises a VL comprising the amino acid sequence of SEQ ID NO: 848 and a VH comprising the amino acid sequence of SEQ ID NO: 1075. In some embodiments, the anti-CD3 antibody, or antigen-binding fragment thereof, comprises ABL-CD3-48_scFv_VH-VL. In some embodiments, the anti-CD3 antibody, or antigen-binding fragment thereof, comprises, from N-terminus to C-terminus, a VH comprising the amino acid sequence of SEQ ID NO: 962; a linker comprising the amino acid sequence of (Gly-Gly-Gly-Gly-Ser)n (SEQ ID NO: 1146), wherein n is 4; and a VL comprising the amino acid sequence of SEQ ID NO: 735. In some embodiments, the anti-CD3 antibody, or antigen-binding fragment thereof, comprises ABL-CD3-48_scFv_VL-VH. In some embodiments, the anti-CD3 antibody, or antigen-binding fragment thereof, comprises, from N-terminus to C-terminus, a VL comprising the amino acid sequence of SEQ ID NO: 735; a linker comprising the amino acid sequence of (Gly-Gly-Gly-Gly-Ser)n (SEQ ID NO: 1146), wherein n is 4; and a VH comprising the amino acid sequence of SEQ ID NO: 962. In some embodiments, the anti-CD3 antibody, or antigen-binding fragment thereof, comprises ABL-CD3-48A_scFv_VH-VL. In some embodiments, the anti-CD3 antibody, or antigen-binding fragment thereof, comprises, from N-terminus to C-terminus, a VH comprising the amino acid sequence of SEQ ID NO: 1075; a linker comprising the amino acid sequence of (Gly-Gly-Gly-Gly-Ser)n (SEQ ID NO: 1146), wherein n is 4; and a VL comprising the amino acid sequence of SEQ ID NO: 848. In some embodiments, the anti-CD3 antibody, or antigen-binding fragment thereof, comprises ABL-CD3-48A_scFv_VL-VH. In some embodiments, the anti-CD3 antibody, or antigen-binding fragment thereof, comprises, from N-terminus to C-terminus, a VL comprising the amino acid sequence of SEQ ID NO: 848; a linker comprising the amino acid sequence of (Gly-Gly-Gly-Gly-Ser)n (SEQ ID NO: 1146), wherein n is 4; and a VH comprising the amino acid sequence of SEQ ID NO: 1075.

In some embodiments, the anti-CD3 antibody, or antigen-binding fragment thereof, comprises the six CDRs of antibody ABL-CD3-49. The anti-CD3 antibody, or antigen-binding fragment thereof, may comprise an LCDR1, an LCDR2, and an LCDR3 comprising the amino acid sequences of SEQ ID NOs: 49, 162, and 275, respectively; and an HCDR1, an HCDR2, and an HCDR3 comprising the amino acid sequences of SEQ ID NOs: 393, 506, and 622, respectively. In some embodiments, the anti-CD3 antibody, or antigen-binding fragment thereof, comprises the six CDRs of antibody ABL-CD3-49A. The anti-CD3 antibody, or antigen-binding fragment thereof, may comprise an LCDR1, an LCDR2, and an LCDR3 comprising the amino acid sequences of SEQ ID NOs: 49, 162, and 342, respectively; and an HCDR1, an HCDR2, and an HCDR3 comprising the amino acid sequences of SEQ ID NOs: 393, 506, and 622, respectively. In some embodiments, the anti-CD3 antibody, or antigen-binding fragment thereof, comprises the VL of antibody ABL-CD3-49. The VL may comprise the amino acid sequence of SEQ ID NO: 736. In some embodiments, the anti-CD3 antibody, or antigen-binding fragment thereof, comprises the VH of antibody ABL-CD3-49. The VH may comprise the amino acid sequence of SEQ ID NO: 963. In some embodiments, the anti-CD3 antibody, or antigen-binding fragment thereof, comprises the VL and VH of antibody ABL-CD3-49. In some embodiments, the anti-CD3 antibody, or antigen-binding fragment thereof, comprises a VL comprising the amino acid sequence of SEQ ID NO: 736 and a VH comprising the amino acid sequence of SEQ ID NO: 963. In some embodiments, the anti-CD3 antibody, or antigen-binding fragment thereof, comprises the VL of antibody ABL-CD3-49A. The VL may comprise the amino acid sequence of SEQ ID NO: 849. In some embodiments, the anti-CD3 antibody, or antigen-binding fragment thereof, comprises the VH of antibody ABL-CD3-49A. The VH may comprise the amino acid sequence of SEQ ID NO: 1076. In some embodiments, the anti-CD3 antibody, or antigen-binding fragment thereof, comprises the VL and VH of antibody ABL-CD3-49A. In some embodiments, the anti-CD3 antibody, or antigen-binding fragment thereof, comprises a VL comprising the amino acid sequence of SEQ ID NO: 849 and a VH comprising the amino acid sequence of SEQ ID NO: 1076. In some embodiments, the anti-CD3 antibody, or antigen-binding fragment thereof, comprises ABL-CD3-49_scFv_VH-VL. In some embodiments, the anti-CD3 antibody, or antigen-binding fragment thereof, comprises, from N-terminus to C-terminus, a VH comprising the amino acid sequence of SEQ ID NO: 963; a linker comprising the amino acid sequence of (Gly-Gly-Gly-Gly-Ser)n (SEQ ID NO: 1146), wherein n is 4; and a VL comprising the amino acid sequence of SEQ ID NO: 736. In some embodiments, the anti-CD3 antibody, or antigen-binding fragment thereof, comprises ABL-CD3-49_scFv_VL-VH. In some embodiments, the anti-CD3 antibody, or antigen-binding fragment thereof, comprises, from N-terminus to C-terminus, a VL comprising the amino acid sequence of SEQ ID NO: 736; a linker comprising the amino acid sequence of (Gly-Gly-Gly-Gly-Ser)n (SEQ ID NO: 1146), wherein n is 4; and a VH comprising the amino acid sequence of SEQ ID NO: 963. In some embodiments, the anti-CD3 antibody, or antigen-binding fragment thereof, comprises ABL-CD3-49A_scFv_VH-VL. In some embodiments, the anti-CD3 antibody, or antigen-binding fragment thereof, comprises the amino acid sequence of SEQ ID NO: 1172. In some embodiments, the anti-CD3 antibody, or antigen-binding fragment thereof, comprises ABL-CD3-49A_scFv_VL-VH. In some embodiments, the anti-CD3 antibody, or antigen-binding fragment thereof, comprises the amino acid sequence of SEQ ID NO: 1213.

In some embodiments, the anti-CD3 antibody, or antigen-binding fragment thereof, comprises the six CDRs of antibody ABL-CD3-50 and ABL-CD3-50A. The anti-CD3 antibody, or antigen-binding fragment thereof, may comprise an LCDR1, an LCDR2, and an LCDR3 comprising the amino acid sequences of SEQ ID NOs: 50, 163, and 276, respectively; and an HCDR1, an HCDR2, and an HCDR3 comprising the amino acid sequences of SEQ ID NOs: 394, 507, and 623, respectively. In some embodiments, the anti-CD3 antibody, or antigen-binding fragment thereof, comprises the VL of antibody ABL-CD3-50. The VL may comprise the amino acid sequence of SEQ ID NO: 737. In some embodiments, the anti-CD3 antibody, or antigen-binding fragment thereof, comprises the VH of antibody ABL-CD3-50. The VH may comprise the amino acid sequence of SEQ ID NO: 964. In some embodiments, the anti-CD3 antibody, or antigen-binding fragment thereof, comprises the VL and VH of antibody ABL-CD3-50. In some embodiments, the anti-CD3 antibody, or antigen-binding fragment thereof, comprises a VL comprising the amino acid sequence of SEQ ID NO: 737 and a VH comprising the amino acid sequence of SEQ ID NO: 964. In some embodiments, the anti-CD3 antibody, or antigen-binding fragment thereof, comprises the VL of antibody ABL-CD3-50A. The VL may comprise the amino acid sequence of SEQ ID NO: 850. In some embodiments, the anti-CD3 antibody, or antigen-binding fragment thereof, comprises the VH of antibody ABL-CD3-50A. The VH may comprise the amino acid sequence of SEQ ID NO: 1077. In some embodiments, the anti-CD3 antibody, or antigen-binding fragment thereof, comprises the VL and VH of antibody ABL-CD3-50A. In some embodiments, the anti-CD3 antibody, or antigen-binding fragment thereof, comprises a VL comprising the amino acid sequence of SEQ ID NO: 850 and a VH comprising the amino acid sequence of SEQ ID NO: 1077. In some embodiments, the anti-CD3 antibody, or antigen-binding fragment thereof, comprises ABL-CD3-50_scFv_VH-VL. In some embodiments, the anti-CD3 antibody, or antigen-binding fragment thereof, comprises, from N-terminus to C-terminus, a VH comprising the amino acid sequence of SEQ ID NO: 964; a linker comprising the amino acid sequence of (Gly-Gly-Gly-Gly-Ser)n (SEQ ID NO: 1146), wherein n is 4; and a VL comprising the amino acid sequence of SEQ ID NO: 737. In some embodiments, the anti-CD3 antibody, or antigen-binding fragment thereof, comprises ABL-CD3-50_scFv_VL-VH. In some embodiments, the anti-CD3 antibody, or antigen-binding fragment thereof, comprises, from N-terminus to C-terminus, a VL comprising the amino acid sequence of SEQ ID NO: 737; a linker comprising the amino acid sequence of (Gly-Gly-Gly-Gly-Ser)n (SEQ ID NO: 1146), wherein n is 4; and a VH comprising the amino acid sequence of SEQ ID NO: 964. In some embodiments, the anti-CD3 antibody, or antigen-binding fragment thereof, comprises ABL-CD3-50A_scFv_VH-VL. In some embodiments, the anti-CD3 antibody, or antigen-binding fragment thereof, comprises, from N-terminus to C-terminus, a VH comprising the amino acid sequence of SEQ ID NO: 1077; a linker comprising the amino acid sequence of (Gly-Gly-Gly-Gly-Ser)n (SEQ ID NO: 1146), wherein n is 4; and a VL comprising the amino acid sequence of SEQ ID NO: 850. In some embodiments, the anti-CD3 antibody, or antigen-binding fragment thereof, comprises ABL-CD3-50A_scFv_VL-VH. In some embodiments, the anti-CD3 antibody, or antigen-binding fragment thereof, comprises, from N-terminus to C-terminus, a VL comprising the amino acid sequence of SEQ ID NO: 850; a linker comprising the amino acid sequence of (Gly-Gly-Gly-Gly-Ser)n (SEQ ID NO: 1146), wherein n is 4; and a VH comprising the amino acid sequence of SEQ ID NO: 1077.

In some embodiments, the anti-CD3 antibody, or antigen-binding fragment thereof, comprises the six CDRs of antibody ABL-CD3-51 and ABL-CD3-51A. The anti-CD3 antibody, or antigen-binding fragment thereof, may comprise an LCDR1, an LCDR2, and an LCDR3 comprising the amino acid sequences of SEQ ID NOs: 51, 164, and 277, respectively; and an HCDR1, an HCDR2, and an HCDR3 comprising the amino acid sequences of SEQ ID NOs: 395, 508, and 624, respectively. In some embodiments, the anti-CD3 antibody, or antigen-binding fragment thereof, comprises the VL of antibody ABL-CD3-51. The VL may comprise the amino acid sequence of SEQ ID NO: 738. In some embodiments, the anti-CD3 antibody, or antigen-binding fragment thereof, comprises the VH of antibody ABL-CD3-51. The VH may comprise the amino acid sequence of SEQ ID NO: 965. In some embodiments, the anti-CD3 antibody, or antigen-binding fragment thereof, comprises the VL and VH of antibody ABL-CD3-51. In some embodiments, the anti-CD3 antibody, or antigen-binding fragment thereof, comprises a VL comprising the amino acid sequence of SEQ ID NO: 738 and a VH comprising the amino acid sequence of SEQ ID NO: 965. In some embodiments, the anti-CD3 antibody, or antigen-binding fragment thereof, comprises the VL of antibody ABL-CD3-51A. The VL may comprise the amino acid sequence of SEQ ID NO: 851. In some embodiments, the anti-CD3 antibody, or antigen-binding fragment thereof, comprises the VH of antibody ABL-CD3-51A. The VH may comprise the amino acid sequence of SEQ ID NO: 1078. In some embodiments, the anti-CD3 antibody, or antigen-binding fragment thereof, comprises the VL and VH of antibody ABL-CD3-51A. In some embodiments, the anti-CD3 antibody, or antigen-binding fragment thereof, comprises a VL comprising the amino acid sequence of SEQ ID NO: 851 and a VH comprising the amino acid sequence of SEQ ID NO: 1078. In some embodiments, the anti-CD3 antibody, or antigen-binding fragment thereof, comprises ABL-CD3-51_scFv_VH-VL. In some embodiments, the anti-CD3 antibody, or antigen-binding fragment thereof, comprises, from N-terminus to C-terminus, a VH comprising the amino acid sequence of SEQ ID NO: 965; a linker comprising the amino acid sequence of (Gly-Gly-Gly-Gly-Ser)n (SEQ ID NO: 1146), wherein n is 4; and a VL comprising the amino acid sequence of SEQ ID NO: 738. In some embodiments, the anti-CD3 antibody, or antigen-binding fragment thereof, comprises ABL-CD3-51_scFv_VL-VH. In some embodiments, the anti-CD3 antibody, or antigen-binding fragment thereof, comprises, from N-terminus to C-terminus, a VL comprising the amino acid sequence of SEQ ID NO: 738; a linker comprising the amino acid sequence of (Gly-Gly-Gly-Gly-Ser)n (SEQ ID NO: 1146), wherein n is 4; and a VH comprising the amino acid sequence of SEQ ID NO: 965. In some embodiments, the anti-CD3 antibody, or antigen-binding fragment thereof, comprises ABL-CD3-51A_scFv_VH-VL. In some embodiments, the anti-CD3 antibody, or antigen-binding fragment thereof, comprises, from N-terminus to C-terminus, a VH comprising the amino acid sequence of SEQ ID NO: 1078; a linker comprising the amino acid sequence of (Gly-Gly-Gly-Gly-Ser)n (SEQ ID NO: 1146), wherein n is 4; and a VL comprising the amino acid sequence of SEQ ID NO: 851. In some embodiments, the anti-CD3 antibody, or antigen-binding fragment thereof, comprises ABL-CD3-51A_scFv_VL-VH. In some embodiments, the anti-CD3 antibody, or antigen-binding fragment thereof, comprises, from N-terminus to C-terminus, a VL comprising the amino acid sequence of SEQ ID NO: 851; a linker comprising the amino acid sequence of (Gly-Gly-Gly-Gly-Ser)n (SEQ ID NO: 1146), wherein n is 4; and a VH comprising the amino acid sequence of SEQ ID NO: 1078.

In some embodiments, the anti-CD3 antibody, or antigen-binding fragment thereof, comprises the six CDRs of antibody ABL-CD3-52 and ABL-CD3-52A. The anti-CD3 antibody, or antigen-binding fragment thereof, may comprise an LCDR1, an LCDR2, and an LCDR3 comprising the amino acid sequences of SEQ ID NOs: 52, 165, and 278, respectively; and an HCDR1, an HCDR2, and an HCDR3 comprising the amino acid sequences of SEQ ID NOs: 396, 509, and 625, respectively. In some embodiments, the anti-CD3 antibody, or antigen-binding fragment thereof, comprises the VL of antibody ABL-CD3-52. The VL may comprise the amino acid sequence of SEQ ID NO: 739. In some embodiments, the anti-CD3 antibody, or antigen-binding fragment thereof, comprises the VH of antibody ABL-CD3-52. The VH may comprise the amino acid sequence of SEQ ID NO: 966. In some embodiments, the anti-CD3 antibody, or antigen-binding fragment thereof, comprises the VL and VH of antibody ABL-CD3-52. In some embodiments, the anti-CD3 antibody, or antigen-binding fragment thereof, comprises a VL comprising the amino acid sequence of SEQ ID NO: 739 and a VH comprising the amino acid sequence of SEQ ID NO: 966. In some embodiments, the anti-CD3 antibody, or antigen-binding fragment thereof, comprises the VL of antibody ABL-CD3-52A. The VL may comprise the amino acid sequence of SEQ ID NO: 852. In some embodiments, the anti-CD3 antibody, or antigen-binding fragment thereof, comprises the VH of antibody ABL-CD3-52A. The VH may comprise the amino acid sequence of SEQ ID NO: 1079. In some embodiments, the anti-CD3 antibody, or antigen-binding fragment thereof, comprises the VL and VH of antibody ABL-CD3-52A. In some embodiments, the anti-CD3 antibody, or antigen-binding fragment thereof, comprises a VL comprising the amino acid sequence of SEQ ID NO: 852 and a VH comprising the amino acid sequence of SEQ ID NO: 1079. In some embodiments, the anti-CD3 antibody, or antigen-binding fragment thereof, comprises ABL-CD3-52_scFv_VH-VL. In some embodiments, the anti-CD3 antibody, or antigen-binding fragment thereof, comprises, from N-terminus to C-terminus, a VH comprising the amino acid sequence of SEQ ID NO: 966; a linker comprising the amino acid sequence of (Gly-Gly-Gly-Gly-Ser)n (SEQ ID NO: 1146), wherein n is 4; and a VL comprising the amino acid sequence of SEQ ID NO: 739. In some embodiments, the anti-CD3 antibody, or antigen-binding fragment thereof, comprises ABL-CD3-52_scFv_VL-VH. In some embodiments, the anti-CD3 antibody, or antigen-binding fragment thereof, comprises, from N-terminus to C-terminus, a VL comprising the amino acid sequence of SEQ ID NO: 739; a linker comprising the amino acid sequence of (Gly-Gly-Gly-Gly-Ser)n (SEQ ID NO: 1146), wherein n is 4; and a VH comprising the amino acid sequence of SEQ ID NO: 966. In some embodiments, the anti-CD3 antibody, or antigen-binding fragment thereof, comprises ABL-CD3-52A_scFv_VH-VL. In some embodiments, the anti-CD3 antibody, or antigen-binding fragment thereof, comprises the amino acid sequence of SEQ ID NO: 1173. In some embodiments, the anti-CD3 antibody, or antigen-binding fragment thereof, comprises ABL-CD3-52A_scFv_VL-VH. In some embodiments, the anti-CD3 antibody, or antigen-binding fragment thereof, comprises the amino acid sequence of SEQ ID NO: 1214.

In some embodiments, the anti-CD3 antibody, or antigen-binding fragment thereof, comprises the six CDRs of antibody ABL-CD3-53 and ABL-CD3-53A. The anti-CD3 antibody, or antigen-binding fragment thereof, may comprise an LCDR1, an LCDR2, and an LCDR3 comprising the amino acid sequences of SEQ ID NOs: 53, 166, and 279, respectively; and an HCDR1, an HCDR2, and an HCDR3 comprising the amino acid sequences of SEQ ID NOs: 397, 510, and 626, respectively. In some embodiments, the anti-CD3 antibody, or antigen-binding fragment thereof, comprises the VL of antibody ABL-CD3-53. The VL may comprise the amino acid sequence of SEQ ID NO: 740. In some embodiments, the anti-CD3 antibody, or antigen-binding fragment thereof, comprises the VH of antibody ABL-CD3-53. The VH may comprise the amino acid sequence of SEQ ID NO: 967. In some embodiments, the anti-CD3 antibody, or antigen-binding fragment thereof, comprises the VL and VH of antibody ABL-CD3-53. In some embodiments, the anti-CD3 antibody, or antigen-binding fragment thereof, comprises a VL comprising the amino acid sequence of SEQ ID NO: 740 and a VH comprising the amino acid sequence of SEQ ID NO: 967. In some embodiments, the anti-CD3 antibody, or antigen-binding fragment thereof, comprises the VL of antibody ABL-CD3-53A. The VL may comprise the amino acid sequence of SEQ ID NO: 853. In some embodiments, the anti-CD3 antibody, or antigen-binding fragment thereof, comprises the VH of antibody ABL-CD3-53A. The VH may comprise the amino acid sequence of SEQ ID NO: 1080. In some embodiments, the anti-CD3 antibody, or antigen-binding fragment thereof, comprises the VL and VH of antibody ABL-CD3-53A. In some embodiments, the anti-CD3 antibody, or antigen-binding fragment thereof, comprises a VL comprising the amino acid sequence of SEQ ID NO: 853 and a VH comprising the amino acid sequence of SEQ ID NO: 1080. In some embodiments, the anti-CD3 antibody, or antigen-binding fragment thereof, comprises ABL-CD3-53_scFv_VH-VL. In some embodiments, the anti-CD3 antibody, or antigen-binding fragment thereof, comprises, from N-terminus to C-terminus, a VH comprising the amino acid sequence of SEQ ID NO: 967; a linker comprising the amino acid sequence of (Gly-Gly-Gly-Gly-Ser)n (SEQ ID NO: 1146), wherein n is 4; and a VL comprising the amino acid sequence of SEQ ID NO: 740. In some embodiments, the anti-CD3 antibody, or antigen-binding fragment thereof, comprises ABL-CD3-53_scFv_VL-VH. In some embodiments, the anti-CD3 antibody, or antigen-binding fragment thereof, comprises, from N-terminus to C-terminus, a VL comprising the amino acid sequence of SEQ ID NO: 740; a linker comprising the amino acid sequence of (Gly-Gly-Gly-Gly-Ser)n (SEQ ID NO: 1146), wherein n is 4; and a VH comprising the amino acid sequence of SEQ ID NO: 967. In some embodiments, the anti-CD3 antibody, or antigen-binding fragment thereof, comprises ABL-CD3-53A_scFv_VH-VL. In some embodiments, the anti-CD3 antibody, or antigen-binding fragment thereof, comprises the amino acid sequence of SEQ ID NO: 1174. In some embodiments, the anti-CD3 antibody, or antigen-binding fragment thereof, comprises ABL-CD3-53A_scFv_VL-VH. In some embodiments, the anti-CD3 antibody, or antigen-binding fragment thereof, comprises the amino acid sequence of SEQ ID NO: 1215.

In some embodiments, the anti-CD3 antibody, or antigen-binding fragment thereof, comprises the six CDRs of antibody ABL-CD3-54 and ABL-CD3-54A. The anti-CD3 antibody, or antigen-binding fragment thereof, may comprise an LCDR1, an LCDR2, and an LCDR3 comprising the amino acid sequences of SEQ ID NOs: 54, 167, and 280, respectively; and an HCDR1, an HCDR2, and an HCDR3 comprising the amino acid sequences of SEQ ID NOs: 398, 511, and 627, respectively. In some embodiments, the anti-CD3 antibody, or antigen-binding fragment thereof, comprises the VL of antibody ABL-CD3-54. The VL may comprise the amino acid sequence of SEQ ID NO: 741. In some embodiments, the anti-CD3 antibody, or antigen-binding fragment thereof, comprises the VH of antibody ABL-CD3-54. The VH may comprise the amino acid sequence of SEQ ID NO: 968. In some embodiments, the anti-CD3 antibody, or antigen-binding fragment thereof, comprises the VL and VH of antibody ABL-CD3-54. In some embodiments, the anti-CD3 antibody, or antigen-binding fragment thereof, comprises a VL comprising the amino acid sequence of SEQ ID NO: 741 and a VH comprising the amino acid sequence of SEQ ID NO: 968. In some embodiments, the anti-CD3 antibody, or antigen-binding fragment thereof, comprises the VL of antibody ABL-CD3-54A. The VL may comprise the amino acid sequence of SEQ ID NO: 854. In some embodiments, the anti-CD3 antibody, or antigen-binding fragment thereof, comprises the VH of antibody ABL-CD3-54A. The VH may comprise the amino acid sequence of SEQ ID NO: 1081. In some embodiments, the anti-CD3 antibody, or antigen-binding fragment thereof, comprises the VL and VH of antibody ABL-CD3-54A. In some embodiments, the anti-CD3 antibody, or antigen-binding fragment thereof, comprises a VL comprising the amino acid sequence of SEQ ID NO: 854 and a VH comprising the amino acid sequence of SEQ ID NO: 1081. In some embodiments, the anti-CD3 antibody, or antigen-binding fragment thereof, comprises ABL-CD3-54_scFv_VH-VL. In some embodiments, the anti-CD3 antibody, or antigen-binding fragment thereof, comprises, from N-terminus to C-terminus, a VH comprising the amino acid sequence of SEQ ID NO: 968; a linker comprising the amino acid sequence of (Gly-Gly-Gly-Gly-Ser)n (SEQ ID NO: 1146), wherein n is 4; and a VL comprising the amino acid sequence of SEQ ID NO: 741. In some embodiments, the anti-CD3 antibody, or antigen-binding fragment thereof, comprises ABL-CD3-54_scFv_VL-VH. In some embodiments, the anti-CD3 antibody, or antigen-binding fragment thereof, comprises, from N-terminus to C-terminus, a VL comprising the amino acid sequence of SEQ ID NO: 741; a linker comprising the amino acid sequence of (Gly-Gly-Gly-Gly-Ser)n (SEQ ID NO: 1146), wherein n is 4; and a VH comprising the amino acid sequence of SEQ ID NO: 968. In some embodiments, the anti-CD3 antibody, or antigen-binding fragment thereof, comprises ABL-CD3-54A_scFv_VH-VL. In some embodiments, the anti-CD3 antibody, or antigen-binding fragment thereof, comprises, from N-terminus to C-terminus, a VH comprising the amino acid sequence of SEQ ID NO: 1081; a linker comprising the amino acid sequence of (Gly-Gly-Gly-Gly-Ser)n (SEQ ID NO: 1146), wherein n is 4; and a VL comprising the amino acid sequence of SEQ ID NO: 854. In some embodiments, the anti-CD3 antibody, or antigen-binding fragment thereof, comprises ABL-CD3-54A_scFv_VL-VH. In some embodiments, the anti-CD3 antibody, or antigen-binding fragment thereof, comprises, from N-terminus to C-terminus, a VL comprising the amino acid sequence of SEQ ID NO: 854; a linker comprising the amino acid sequence of (Gly-Gly-Gly-Gly-Ser)n (SEQ ID NO: 1146), wherein n is 4; and a VH comprising the amino acid sequence of SEQ ID NO: 1081.

In some embodiments, the anti-CD3 antibody, or antigen-binding fragment thereof, comprises the six CDRs of antibody ABL-CD3-55 and ABL-CD3-55A. The anti-CD3 antibody, or antigen-binding fragment thereof, may comprise an LCDR1, an LCDR2, and an LCDR3 comprising the amino acid sequences of SEQ ID NOs: 55, 168, and 281, respectively; and an HCDR1, an HCDR2, and an HCDR3 comprising the amino acid sequences of SEQ ID NOs: 399, 512, and 628, respectively. In some embodiments, the anti-CD3 antibody, or antigen-binding fragment thereof, comprises the VL of antibody ABL-CD3-55. The VL may comprise the amino acid sequence of SEQ ID NO: 742. In some embodiments, the anti-CD3 antibody, or antigen-binding fragment thereof, comprises the VH of antibody ABL-CD3-55. The VH may comprise the amino acid sequence of SEQ ID NO: 969. In some embodiments, the anti-CD3 antibody, or antigen-binding fragment thereof, comprises the VL and VH of antibody ABL-CD3-55. In some embodiments, the anti-CD3 antibody, or antigen-binding fragment thereof, comprises a VL comprising the amino acid sequence of SEQ ID NO: 742 and a VH comprising the amino acid sequence of SEQ ID NO: 969. In some embodiments, the anti-CD3 antibody, or antigen-binding fragment thereof, comprises the VL of antibody ABL-CD3-55A. The VL may comprise the amino acid sequence of SEQ ID NO: 855. In some embodiments, the anti-CD3 antibody, or antigen-binding fragment thereof, comprises the VH of antibody ABL-CD3-55A. The VH may comprise the amino acid sequence of SEQ ID NO: 1082. In some embodiments, the anti-CD3 antibody, or antigen-binding fragment thereof, comprises the VL and VH of antibody ABL-CD3-55A. In some embodiments, the anti-CD3 antibody, or antigen-binding fragment thereof, comprises a VL comprising the amino acid sequence of SEQ ID NO: 855 and a VH comprising the amino acid sequence of SEQ ID NO: 1082. In some embodiments, the anti-CD3 antibody, or antigen-binding fragment thereof, comprises ABL-CD3-55_scFv_VH-VL. In some embodiments, the anti-CD3 antibody, or antigen-binding fragment thereof, comprises, from N-terminus to C-terminus, a VH comprising the amino acid sequence of SEQ ID NO: 969; a linker comprising the amino acid sequence of (Gly-Gly-Gly-Gly-Ser)n (SEQ ID NO: 1146), wherein n is 4; and a VL comprising the amino acid sequence of SEQ ID NO: 742. In some embodiments, the anti-CD3 antibody, or antigen-binding fragment thereof, comprises ABL-CD3-55_scFv_VL-VH. In some embodiments, the anti-CD3 antibody, or antigen-binding fragment thereof, comprises, from N-terminus to C-terminus, a VL comprising the amino acid sequence of SEQ ID NO: 742; a linker comprising the amino acid sequence of (Gly-Gly-Gly-Gly-Ser)n (SEQ ID NO: 1146), wherein n is 4; and a VH comprising the amino acid sequence of SEQ ID NO: 969. In some embodiments, the anti-CD3 antibody, or antigen-binding fragment thereof, comprises ABL-CD3-55A_scFv_VH-VL. In some embodiments, the anti-CD3 antibody, or antigen-binding fragment thereof, comprises, from N-terminus to C-terminus, a VH comprising the amino acid sequence of SEQ ID NO: 1082; a linker comprising the amino acid sequence of (Gly-Gly-Gly-Gly-Ser)n (SEQ ID NO: 1146), wherein n is 4; and a VL comprising the amino acid sequence of SEQ ID NO: 855. In some embodiments, the anti-CD3 antibody, or antigen-binding fragment thereof, comprises ABL-CD3-55A_scFv_VL-VH. In some embodiments, the anti-CD3 antibody, or antigen-binding fragment thereof, comprises, from N-terminus to C-terminus, a VL comprising the amino acid sequence of SEQ ID NO: 855; a linker comprising the amino acid sequence of (Gly-Gly-Gly-Gly-Ser)n (SEQ ID NO: 1146), wherein n is 4; and a VH comprising the amino acid sequence of SEQ ID NO: 1082.

In some embodiments, the anti-CD3 antibody, or antigen-binding fragment thereof, comprises the six CDRs of antibody ABL-CD3-56 and ABL-CD3-56A. The anti-CD3 antibody, or antigen-binding fragment thereof, may comprise an LCDR1, an LCDR2, and an LCDR3 comprising the amino acid sequences of SEQ ID NOs: 56, 169, and 282, respectively; and an HCDR1, an HCDR2, and an HCDR3 comprising the amino acid sequences of SEQ ID NOs: 400, 513, and 629, respectively. In some embodiments, the anti-CD3 antibody, or antigen-binding fragment thereof, comprises the VL of antibody ABL-CD3-56. The VL may comprise the amino acid sequence of SEQ ID NO: 743. In some embodiments, the anti-CD3 antibody, or antigen-binding fragment thereof, comprises the VH of antibody ABL-CD3-56. The VH may comprise the amino acid sequence of SEQ ID NO: 970. In some embodiments, the anti-CD3 antibody, or antigen-binding fragment thereof, comprises the VL and VH of antibody ABL-CD3-56. In some embodiments, the anti-CD3 antibody, or antigen-binding fragment thereof, comprises a VL comprising the amino acid sequence of SEQ ID NO: 743 and a VH comprising the amino acid sequence of SEQ ID NO: 970. In some embodiments, the anti-CD3 antibody, or antigen-binding fragment thereof, comprises the VL of antibody ABL-CD3-56A. The VL may comprise the amino acid sequence of SEQ ID NO: 856. In some embodiments, the anti-CD3 antibody, or antigen-binding fragment thereof, comprises the VH of antibody ABL-CD3-56A. The VH may comprise the amino acid sequence of SEQ ID NO: 1083. In some embodiments, the anti-CD3 antibody, or antigen-binding fragment thereof, comprises the VL and VH of antibody ABL-CD3-56A. In some embodiments, the anti-CD3 antibody, or antigen-binding fragment thereof, comprises a VL comprising the amino acid sequence of SEQ ID NO: 856 and a VH comprising the amino acid sequence of SEQ ID NO: 1083. In some embodiments, the anti-CD3 antibody, or antigen-binding fragment thereof, comprises ABL-CD3-56_scFv_VH-VL. In some embodiments, the anti-CD3 antibody, or antigen-binding fragment thereof, comprises, from N-terminus to C-terminus, a VH comprising the amino acid sequence of SEQ ID NO: 970; a linker comprising the amino acid sequence of (Gly-Gly-Gly-Gly-Ser)n (SEQ ID NO: 1146), wherein n is 4; and a VL comprising the amino acid sequence of SEQ ID NO: 743. In some embodiments, the anti-CD3 antibody, or antigen-binding fragment thereof, comprises ABL-CD3-56_scFv_VL-VH. In some embodiments, the anti-CD3 antibody, or antigen-binding fragment thereof, comprises, from N-terminus to C-terminus, a VL comprising the amino acid sequence of SEQ ID NO: 743; a linker comprising the amino acid sequence of (Gly-Gly-Gly-Gly-Ser)n (SEQ ID NO: 1146), wherein n is 4; and a VH comprising the amino acid sequence of SEQ ID NO: 970. In some embodiments, the anti-CD3 antibody, or antigen-binding fragment thereof, comprises ABL-CD3-56A_scFv_VH-VL. In some embodiments, the anti-CD3 antibody, or antigen-binding fragment thereof, comprises the amino acid sequence of SEQ ID NO: 1175. In some embodiments, the anti-CD3 antibody, or antigen-binding fragment thereof, comprises ABL-CD3-56A_scFv_VL-VH. In some embodiments, the anti-CD3 antibody, or antigen-binding fragment thereof, comprises the amino acid sequence of SEQ ID NO: 1216.

In some embodiments, the anti-CD3 antibody, or antigen-binding fragment thereof, comprises the six CDRs of antibody ABL-CD3-57 and ABL-CD3-57A. The anti-CD3 antibody, or antigen-binding fragment thereof, may comprise an LCDR1, an LCDR2, and an LCDR3 comprising the amino acid sequences of SEQ ID NOs: 57, 170, and 283, respectively; and an HCDR1, an HCDR2, and an HCDR3 comprising the amino acid sequences of SEQ ID NOs: 401, 514, and 630, respectively. In some embodiments, the anti-CD3 antibody, or antigen-binding fragment thereof, comprises the VL of antibody ABL-CD3-57. The VL may comprise the amino acid sequence of SEQ ID NO: 744. In some embodiments, the anti-CD3 antibody, or antigen-binding fragment thereof, comprises the VH of antibody ABL-CD3-57. The VH may comprise the amino acid sequence of SEQ ID NO: 971. In some embodiments, the anti-CD3 antibody, or antigen-binding fragment thereof, comprises the VL and VH of antibody ABL-CD3-57. In some embodiments, the anti-CD3 antibody, or antigen-binding fragment thereof, comprises a VL comprising the amino acid sequence of SEQ ID NO: 744 and a VH comprising the amino acid sequence of SEQ ID NO: 971. In some embodiments, the anti-CD3 antibody, or antigen-binding fragment thereof, comprises the VL of antibody ABL-CD3-57A. The VL may comprise the amino acid sequence of SEQ ID NO: 857. In some embodiments, the anti-CD3 antibody, or antigen-binding fragment thereof, comprises the VH of antibody ABL-CD3-57A. The VH may comprise the amino acid sequence of SEQ ID NO: 1084. In some embodiments, the anti-CD3 antibody, or antigen-binding fragment thereof, comprises the VL and VH of antibody ABL-CD3-57A. In some embodiments, the anti-CD3 antibody, or antigen-binding fragment thereof, comprises a VL comprising the amino acid sequence of SEQ ID NO: 857 and a VH comprising the amino acid sequence of SEQ ID NO: 1084. In some embodiments, the anti-CD3 antibody, or antigen-binding fragment thereof, comprises ABL-CD3-57_scFv_VH-VL. In some embodiments, the anti-CD3 antibody, or antigen-binding fragment thereof, comprises, from N-terminus to C-terminus, a VH comprising the amino acid sequence of SEQ ID NO: 971; a linker comprising the amino acid sequence of (Gly-Gly-Gly-Gly-Ser)n (SEQ ID NO: 1146), wherein n is 4; and a VL comprising the amino acid sequence of SEQ ID NO: 744. In some embodiments, the anti-CD3 antibody, or antigen-binding fragment thereof, comprises ABL-CD3-57_scFv_VL-VH. In some embodiments, the anti-CD3 antibody, or antigen-binding fragment thereof, comprises, from N-terminus to C-terminus, a VL comprising the amino acid sequence of SEQ ID NO: 744; a linker comprising the amino acid sequence of (Gly-Gly-Gly-Gly-Ser)n (SEQ ID NO: 1146), wherein n is 4; and a VH comprising the amino acid sequence of SEQ ID NO: 971. In some embodiments, the anti-CD3 antibody, or antigen-binding fragment thereof, comprises ABL-CD3-57A_scFv_VH-VL. In some embodiments, the anti-CD3 antibody, or antigen-binding fragment thereof, comprises the amino acid sequence of SEQ ID NO: 1176. In some embodiments, the anti-CD3 antibody, or antigen-binding fragment thereof, comprises ABL-CD3-57A_scFv_VL-VH. In some embodiments, the anti-CD3 antibody, or antigen-binding fragment thereof, comprises the amino acid sequence of SEQ ID NO: 1217.

In some embodiments, the anti-CD3 antibody, or antigen-binding fragment thereof, comprises the six CDRs of antibody ABL-CD3-58 and ABL-CD3-58A. The anti-CD3 antibody, or antigen-binding fragment thereof, may comprise an LCDR1, an LCDR2, and an LCDR3 comprising the amino acid sequences of SEQ ID NOs: 58, 171, and 284, respectively; and an HCDR1, an HCDR2, and an HCDR3 comprising the amino acid sequences of SEQ ID NOs: 402, 515, and 631, respectively. In some embodiments, the anti-CD3 antibody, or antigen-binding fragment thereof, comprises the VL of antibody ABL-CD3-58. The VL may comprise the amino acid sequence of SEQ ID NO: 745. In some embodiments, the anti-CD3 antibody, or antigen-binding fragment thereof, comprises the VH of antibody ABL-CD3-58. The VH may comprise the amino acid sequence of SEQ ID NO: 972. In some embodiments, the anti-CD3 antibody, or antigen-binding fragment thereof, comprises the VL and VH of antibody ABL-CD3-58. In some embodiments, the anti-CD3 antibody, or antigen-binding fragment thereof, comprises a VL comprising the amino acid sequence of SEQ ID NO: 745 and a VH comprising the amino acid sequence of SEQ ID NO: 972. In some embodiments, the anti-CD3 antibody, or antigen-binding fragment thereof, comprises the VL of antibody ABL-CD3-58A. The VL may comprise the amino acid sequence of SEQ ID NO: 858. In some embodiments, the anti-CD3 antibody, or antigen-binding fragment thereof, comprises the VH of antibody ABL-CD3-58A. The VH may comprise the amino acid sequence of SEQ ID NO: 1085. In some embodiments, the anti-CD3 antibody, or antigen-binding fragment thereof, comprises the VL and VH of antibody ABL-CD3-58A. In some embodiments, the anti-CD3 antibody, or antigen-binding fragment thereof, comprises a VL comprising the amino acid sequence of SEQ ID NO: 858 and a VH comprising the amino acid sequence of SEQ ID NO: 1085. In some embodiments, the anti-CD3 antibody, or antigen-binding fragment thereof, comprises ABL-CD3-58_scFv_VH-VL. In some embodiments, the anti-CD3 antibody, or antigen-binding fragment thereof, comprises, from N-terminus to C-terminus, a VH comprising the amino acid sequence of SEQ ID NO: 972; a linker comprising the amino acid sequence of (Gly-Gly-Gly-Gly-Ser)n (SEQ ID NO: 1146), wherein n is 4; and a VL comprising the amino acid sequence of SEQ ID NO: 745. In some embodiments, the anti-CD3 antibody, or antigen-binding fragment thereof, comprises ABL-CD3-58_scFv_VL-VH. In some embodiments, the anti-CD3 antibody, or antigen-binding fragment thereof, comprises, from N-terminus to C-terminus, a VL comprising the amino acid sequence of SEQ ID NO: 745; a linker comprising the amino acid sequence of (Gly-Gly-Gly-Gly-Ser)n (SEQ ID NO: 1146), wherein n is 4; and a VH comprising the amino acid sequence of SEQ ID NO: 972. In some embodiments, the anti-CD3 antibody, or antigen-binding fragment thereof, comprises ABL-CD3-58A_scFv_VH-VL. In some embodiments, the anti-CD3 antibody, or antigen-binding fragment thereof, comprises, from N-terminus to C-terminus, a VH comprising the amino acid sequence of SEQ ID NO: 1085; a linker comprising the amino acid sequence of (Gly-Gly-Gly-Gly-Ser)n (SEQ ID NO: 1146), wherein n is 4; and a VL comprising the amino acid sequence of SEQ ID NO: 858. In some embodiments, the anti-CD3 antibody, or antigen-binding fragment thereof, comprises ABL-CD3-58A_scFv_VL-VH. In some embodiments, the anti-CD3 antibody, or antigen-binding fragment thereof, comprises, from N-terminus to C-terminus, a VL comprising the amino acid sequence of SEQ ID NO: 858; a linker comprising the amino acid sequence of (Gly-Gly-Gly-Gly-Ser)n (SEQ ID NO: 1146), wherein n is 4; and a VH comprising the amino acid sequence of SEQ ID NO: 1085.

In some embodiments, the anti-CD3 antibody, or antigen-binding fragment thereof, comprises the six CDRs of antibody ABL-CD3-59 and ABL-CD3-59A. The anti-CD3 antibody, or antigen-binding fragment thereof, may comprise an LCDR1, an LCDR2, and an LCDR3 comprising the amino acid sequences of SEQ ID NOs: 59, 172, and 285, respectively; and an HCDR1, an HCDR2, and an HCDR3 comprising the amino acid sequences of SEQ ID NOs: 403, 516, and 632, respectively. In some embodiments, the anti-CD3 antibody, or antigen-binding fragment thereof, comprises the VL of antibody ABL-CD3-59. The VL may comprise the amino acid sequence of SEQ ID NO: 746. In some embodiments, the anti-CD3 antibody, or antigen-binding fragment thereof, comprises the VH of antibody ABL-CD3-59. The VH may comprise the amino acid sequence of SEQ ID NO: 973. In some embodiments, the anti-CD3 antibody, or antigen-binding fragment thereof, comprises the VL and VH of antibody ABL-CD3-59. In some embodiments, the anti-CD3 antibody, or antigen-binding fragment thereof, comprises a VL comprising the amino acid sequence of SEQ ID NO: 746 and a VH comprising the amino acid sequence of SEQ ID NO: 973. In some embodiments, the anti-CD3 antibody, or antigen-binding fragment thereof, comprises the VL of antibody ABL-CD3-59A. The VL may comprise the amino acid sequence of SEQ ID NO: 859. In some embodiments, the anti-CD3 antibody, or antigen-binding fragment thereof, comprises the VH of antibody ABL-CD3-59A. The VH may comprise the amino acid sequence of SEQ ID NO: 1086. In some embodiments, the anti-CD3 antibody, or antigen-binding fragment thereof, comprises the VL and VH of antibody ABL-CD3-59A. In some embodiments, the anti-CD3 antibody, or antigen-binding fragment thereof, comprises a VL comprising the amino acid sequence of SEQ ID NO: 859 and a VH comprising the amino acid sequence of SEQ ID NO: 1086. In some embodiments, the anti-CD3 antibody, or antigen-binding fragment thereof, comprises ABL-CD3-59_scFv_VH-VL. In some embodiments, the anti-CD3 antibody, or antigen-binding fragment thereof, comprises, from N-terminus to C-terminus, a VH comprising the amino acid sequence of SEQ ID NO: 973; a linker comprising the amino acid sequence of (Gly-Gly-Gly-Gly-Ser)n (SEQ ID NO: 1146), wherein n is 4; and a VL comprising the amino acid sequence of SEQ ID NO: 746. In some embodiments, the anti-CD3 antibody, or antigen-binding fragment thereof, comprises ABL-CD3-59_scFv_VL-VH. In some embodiments, the anti-CD3 antibody, or antigen-binding fragment thereof, comprises, from N-terminus to C-terminus, a VL comprising the amino acid sequence of SEQ ID NO: 746; a linker comprising the amino acid sequence of (Gly-Gly-Gly-Gly-Ser)n (SEQ ID NO: 1146), wherein n is 4; and a VH comprising the amino acid sequence of SEQ ID NO: 973. In some embodiments, the anti-CD3 antibody, or antigen-binding fragment thereof, comprises ABL-CD3-59A_scFv_VH-VL. In some embodiments, the anti-CD3 antibody, or antigen-binding fragment thereof, comprises, from N-terminus to C-terminus, a VH comprising the amino acid sequence of SEQ ID NO: 1086; a linker comprising the amino acid sequence of (Gly-Gly-Gly-Gly-Ser)n (SEQ ID NO: 1146), wherein n is 4; and a VL comprising the amino acid sequence of SEQ ID NO: 859. In some embodiments, the anti-CD3 antibody, or antigen-binding fragment thereof, comprises ABL-CD3-59A_scFv_VL-VH. In some embodiments, the anti-CD3 antibody, or antigen-binding fragment thereof, comprises, from N-terminus to C-terminus, a VL comprising the amino acid sequence of SEQ ID NO: 859; a linker comprising the amino acid sequence of (Gly-Gly-Gly-Gly-Ser)n (SEQ ID NO: 1146), wherein n is 4; and a VH comprising the amino acid sequence of SEQ ID NO: 1086.

In some embodiments, the anti-CD3 antibody, or antigen-binding fragment thereof, comprises the six CDRs of antibody ABL-CD3-60 and ABL-CD3-60A. The anti-CD3 antibody, or antigen-binding fragment thereof, may comprise an LCDR1, an LCDR2, and an LCDR3 comprising the amino acid sequences of SEQ ID NOs: 60, 173, and 286, respectively; and an HCDR1, an HCDR2, and an HCDR3 comprising the amino acid sequences of SEQ ID NOs: 404, 517, and 633, respectively. In some embodiments, the anti-CD3 antibody, or antigen-binding fragment thereof, comprises the VL of antibody ABL-CD3-60. The VL may comprise the amino acid sequence of SEQ ID NO: 747. In some embodiments, the anti-CD3 antibody, or antigen-binding fragment thereof, comprises the VH of antibody ABL-CD3-60. The VH may comprise the amino acid sequence of SEQ ID NO: 974. In some embodiments, the anti-CD3 antibody, or antigen-binding fragment thereof, comprises the VL and VH of antibody ABL-CD3-60. In some embodiments, the anti-CD3 antibody, or antigen-binding fragment thereof, comprises a VL comprising the amino acid sequence of SEQ ID NO: 747 and a VH comprising the amino acid sequence of SEQ ID NO: 974. In some embodiments, the anti-CD3 antibody, or antigen-binding fragment thereof, comprises the VL of antibody ABL-CD3-60A. The VL may comprise the amino acid sequence of SEQ ID NO: 860. In some embodiments, the anti-CD3 antibody, or antigen-binding fragment thereof, comprises the VH of antibody ABL-CD3-60A. The VH may comprise the amino acid sequence of SEQ ID NO: 1087. In some embodiments, the anti-CD3 antibody, or antigen-binding fragment thereof, comprises the VL and VH of antibody ABL-CD3-60A. In some embodiments, the anti-CD3 antibody, or antigen-binding fragment thereof, comprises a VL comprising the amino acid sequence of SEQ ID NO: 860 and a VH comprising the amino acid sequence of SEQ ID NO: 1087. In some embodiments, the anti-CD3 antibody, or antigen-binding fragment thereof, comprises ABL-CD3-60_scFv_VH-VL. In some embodiments, the anti-CD3 antibody, or antigen-binding fragment thereof, comprises, from N-terminus to C-terminus, a VH comprising the amino acid sequence of SEQ ID NO: 974; a linker comprising the amino acid sequence of (Gly-Gly-Gly-Gly-Ser)n (SEQ ID NO: 1146), wherein n is 4; and a VL comprising the amino acid sequence of SEQ ID NO: 747. In some embodiments, the anti-CD3 antibody, or antigen-binding fragment thereof, comprises ABL-CD3-60_scFv_VL-VH. In some embodiments, the anti-CD3 antibody, or antigen-binding fragment thereof, comprises, from N-terminus to C-terminus, a VL comprising the amino acid sequence of SEQ ID NO: 747; a linker comprising the amino acid sequence of (Gly-Gly-Gly-Gly-Ser)n (SEQ ID NO: 1146), wherein n is 4; and a VH comprising the amino acid sequence of SEQ ID NO: 974. In some embodiments, the anti-CD3 antibody, or antigen-binding fragment thereof, comprises ABL-CD3-60A_scFv_VH-VL. In some embodiments, the anti-CD3 antibody, or antigen-binding fragment thereof, comprises, from N-terminus to C-terminus, a VH comprising the amino acid sequence of SEQ ID NO: 1087; a linker comprising the amino acid sequence of (Gly-Gly-Gly-Gly-Ser)n (SEQ ID NO: 1146), wherein n is 4; and a VL comprising the amino acid sequence of SEQ ID NO: 860. In some embodiments, the anti-CD3 antibody, or antigen-binding fragment thereof, comprises ABL-CD3-60A_scFv_VL-VH. In some embodiments, the anti-CD3 antibody, or antigen-binding fragment thereof, comprises, from N-terminus to C-terminus, a VL comprising the amino acid sequence of SEQ ID NO: 860; a linker comprising the amino acid sequence of (Gly-Gly-Gly-Gly-Ser)n (SEQ ID NO: 1146), wherein n is 4; and a VH comprising the amino acid sequence of SEQ ID NO: 1087.

In some embodiments, the anti-CD3 antibody, or antigen-binding fragment thereof, comprises the six CDRs of antibody ABL-CD3-61 and ABL-CD3-61A. The anti-CD3 antibody, or antigen-binding fragment thereof, may comprise an LCDR1, an LCDR2, and an LCDR3 comprising the amino acid sequences of SEQ ID NOs: 61, 174, and 287, respectively; and an HCDR1, an HCDR2, and an HCDR3 comprising the amino acid sequences of SEQ ID NOs: 405, 518, and 634, respectively. In some embodiments, the anti-CD3 antibody, or antigen-binding fragment thereof, comprises the VL of antibody ABL-CD3-61. The VL may comprise the amino acid sequence of SEQ ID NO: 748. In some embodiments, the anti-CD3 antibody, or antigen-binding fragment thereof, comprises the VH of antibody ABL-CD3-61. The VH may comprise the amino acid sequence of SEQ ID NO: 975. In some embodiments, the anti-CD3 antibody, or antigen-binding fragment thereof, comprises the VL and VH of antibody ABL-CD3-61. In some embodiments, the anti-CD3 antibody, or antigen-binding fragment thereof, comprises a VL comprising the amino acid sequence of SEQ ID NO: 748 and a VH comprising the amino acid sequence of SEQ ID NO: 975. In some embodiments, the anti-CD3 antibody, or antigen-binding fragment thereof, comprises the VL of antibody ABL-CD3-61A. The VL may comprise the amino acid sequence of SEQ ID NO: 861. In some embodiments, the anti-CD3 antibody, or antigen-binding fragment thereof, comprises the VH of antibody ABL-CD3-61A. The VH may comprise the amino acid sequence of SEQ ID NO: 1088. In some embodiments, the anti-CD3 antibody, or antigen-binding fragment thereof, comprises the VL and VH of antibody ABL-CD3-61A. In some embodiments, the anti-CD3 antibody, or antigen-binding fragment thereof, comprises a VL comprising the amino acid sequence of SEQ ID NO: 861 and a VH comprising the amino acid sequence of SEQ ID NO: 1088. In some embodiments, the anti-CD3 antibody, or antigen-binding fragment thereof, comprises ABL-CD3-61_scFv_VH-VL. In some embodiments, the anti-CD3 antibody, or antigen-binding fragment thereof, comprises, from N-terminus to C-terminus, a VH comprising the amino acid sequence of SEQ ID NO: 975; a linker comprising the amino acid sequence of (Gly-Gly-Gly-Gly-Ser)n (SEQ ID NO: 1146), wherein n is 4; and a VL comprising the amino acid sequence of SEQ ID NO: 748. In some embodiments, the anti-CD3 antibody, or antigen-binding fragment thereof, comprises ABL-CD3-61_scFv_VL-VH. In some embodiments, the anti-CD3 antibody, or antigen-binding fragment thereof, comprises, from N-terminus to C-terminus, a VL comprising the amino acid sequence of SEQ ID NO: 748; a linker comprising the amino acid sequence of (Gly-Gly-Gly-Gly-Ser)n (SEQ ID NO: 1146), wherein n is 4; and a VH comprising the amino acid sequence of SEQ ID NO: 975. In some embodiments, the anti-CD3 antibody, or antigen-binding fragment thereof, comprises ABL-CD3-61A_scFv_VH-VL. In some embodiments, the anti-CD3 antibody, or antigen-binding fragment thereof, comprises the amino acid sequence of SEQ ID NO: 1177. In some embodiments, the anti-CD3 antibody, or antigen-binding fragment thereof, comprises ABL-CD3-61A_scFv_VL-VH. In some embodiments, the anti-CD3 antibody, or antigen-binding fragment thereof, comprises the amino acid sequence of SEQ ID NO: 1218.

In some embodiments, the anti-CD3 antibody, or antigen-binding fragment thereof, comprises the six CDRs of antibody ABL-CD3-62 and ABL-CD3-62A. The anti-CD3 antibody, or antigen-binding fragment thereof, may comprise an LCDR1, an LCDR2, and an LCDR3 comprising the amino acid sequences of SEQ ID NOs: 62, 175, and 288, respectively; and an HCDR1, an HCDR2, and an HCDR3 comprising the amino acid sequences of SEQ ID NOs: 406, 519, and 635, respectively. In some embodiments, the anti-CD3 antibody, or antigen-binding fragment thereof, comprises the VL of antibody ABL-CD3-62. The VL may comprise the amino acid sequence of SEQ ID NO: 749. In some embodiments, the anti-CD3 antibody, or antigen-binding fragment thereof, comprises the VH of antibody ABL-CD3-62. The VH may comprise the amino acid sequence of SEQ ID NO: 976. In some embodiments, the anti-CD3 antibody, or antigen-binding fragment thereof, comprises the VL and VH of antibody ABL-CD3-62. In some embodiments, the anti-CD3 antibody, or antigen-binding fragment thereof, comprises a VL comprising the amino acid sequence of SEQ ID NO: 749 and a VH comprising the amino acid sequence of SEQ ID NO: 976. In some embodiments, the anti-CD3 antibody, or antigen-binding fragment thereof, comprises the VL of antibody ABL-CD3-62A. The VL may comprise the amino acid sequence of SEQ ID NO: 862. In some embodiments, the anti-CD3 antibody, or antigen-binding fragment thereof, comprises the VH of antibody ABL-CD3-62A. The VH may comprise the amino acid sequence of SEQ ID NO: 1089. In some embodiments, the anti-CD3 antibody, or antigen-binding fragment thereof, comprises the VL and VH of antibody ABL-CD3-62A. In some embodiments, the anti-CD3 antibody, or antigen-binding fragment thereof, comprises a VL comprising the amino acid sequence of SEQ ID NO: 862 and a VH comprising the amino acid sequence of SEQ ID NO: 1089. In some embodiments, the anti-CD3 antibody, or antigen-binding fragment thereof, comprises ABL-CD3-62_scFv_VH-VL. In some embodiments, the anti-CD3 antibody, or antigen-binding fragment thereof, comprises, from N-terminus to C-terminus, a VH comprising the amino acid sequence of SEQ ID NO: 976; a linker comprising the amino acid sequence of (Gly-Gly-Gly-Gly-Ser)n (SEQ ID NO: 1146), wherein n is 4; and a VL comprising the amino acid sequence of SEQ ID NO: 749. In some embodiments, the anti-CD3 antibody, or antigen-binding fragment thereof, comprises ABL-CD3-62_scFv_VL-VH. In some embodiments, the anti-CD3 antibody, or antigen-binding fragment thereof, comprises, from N-terminus to C-terminus, a VL comprising the amino acid sequence of SEQ ID NO: 749; a linker comprising the amino acid sequence of (Gly-Gly-Gly-Gly-Ser)n (SEQ ID NO: 1146), wherein n is 4; and a VH comprising the amino acid sequence of SEQ ID NO: 976. In some embodiments, the anti-CD3 antibody, or antigen-binding fragment thereof, comprises ABL-CD3-62A_scFv_VH-VL. In some embodiments, the anti-CD3 antibody, or antigen-binding fragment thereof, comprises the amino acid sequence of SEQ ID NO: 1178. In some embodiments, the anti-CD3 antibody, or antigen-binding fragment thereof, comprises ABL-CD3-62A_scFv_VL-VH. In some embodiments, the anti-CD3 antibody, or antigen-binding fragment thereof, comprises the amino acid sequence of SEQ ID NO: 1219.

In some embodiments, the anti-CD3 antibody, or antigen-binding fragment thereof, comprises the six CDRs of antibody ABL-CD3-63 and ABL-CD3-63A. The anti-CD3 antibody, or antigen-binding fragment thereof, may comprise an LCDR1, an LCDR2, and an LCDR3 comprising the amino acid sequences of SEQ ID NOs: 63, 176, and 289, respectively; and an HCDR1, an HCDR2, and an HCDR3 comprising the amino acid sequences of SEQ ID NOs: 407, 520, and 636, respectively. In some embodiments, the anti-CD3 antibody, or antigen-binding fragment thereof, comprises the VL of antibody ABL-CD3-63. The VL may comprise the amino acid sequence of SEQ ID NO: 750. In some embodiments, the anti-CD3 antibody, or antigen-binding fragment thereof, comprises the VH of antibody ABL-CD3-63. The VH may comprise the amino acid sequence of SEQ ID NO: 977. In some embodiments, the anti-CD3 antibody, or antigen-binding fragment thereof, comprises the VL and VH of antibody ABL-CD3-63. In some embodiments, the anti-CD3 antibody, or antigen-binding fragment thereof, comprises a VL comprising the amino acid sequence of SEQ ID NO: 750 and a VH comprising the amino acid sequence of SEQ ID NO: 977. In some embodiments, the anti-CD3 antibody, or antigen-binding fragment thereof, comprises the VL of antibody ABL-CD3-63A. The VL may comprise the amino acid sequence of SEQ ID NO: 863. In some embodiments, the anti-CD3 antibody, or antigen-binding fragment thereof, comprises the VH of antibody ABL-CD3-63A. The VH may comprise the amino acid sequence of SEQ ID NO: 1090. In some embodiments, the anti-CD3 antibody, or antigen-binding fragment thereof, comprises the VL and VH of antibody ABL-CD3-63A. In some embodiments, the anti-CD3 antibody, or antigen-binding fragment thereof, comprises a VL comprising the amino acid sequence of SEQ ID NO: 863 and a VH comprising the amino acid sequence of SEQ ID NO: 1090. In some embodiments, the anti-CD3 antibody, or antigen-binding fragment thereof, comprises ABL-CD3-63_scFv_VH-VL. In some embodiments, the anti-CD3 antibody, or antigen-binding fragment thereof, comprises, from N-terminus to C-terminus, a VH comprising the amino acid sequence of SEQ ID NO: 977; a linker comprising the amino acid sequence of (Gly-Gly-Gly-Gly-Ser)n (SEQ ID NO: 1146), wherein n is 4; and a VL comprising the amino acid sequence of SEQ ID NO: 750. In some embodiments, the anti-CD3 antibody, or antigen-binding fragment thereof, comprises ABL-CD3-63_scFv_VL-VH. In some embodiments, the anti-CD3 antibody, or antigen-binding fragment thereof, comprises, from N-terminus to C-terminus, a VL comprising the amino acid sequence of SEQ ID NO: 750; a linker comprising the amino acid sequence of (Gly-Gly-Gly-Gly-Ser)n (SEQ ID NO: 1146), wherein n is 4; and a VH comprising the amino acid sequence of SEQ ID NO: 977. In some embodiments, the anti-CD3 antibody, or antigen-binding fragment thereof, comprises ABL-CD3-63A_scFv_VH-VL. In some embodiments, the anti-CD3 antibody, or antigen-binding fragment thereof, comprises the amino acid sequence of SEQ ID NO: 1179. In some embodiments, the anti-CD3 antibody, or antigen-binding fragment thereof, comprises ABL-CD3-63A_scFv_VL-VH. In some embodiments, the anti-CD3 antibody, or antigen-binding fragment thereof, comprises the amino acid sequence of SEQ ID NO: 1220.

In some embodiments, the anti-CD3 antibody, or antigen-binding fragment thereof, comprises the six CDRs of antibody ABL-CD3-64 and ABL-CD3-64A. The anti-CD3 antibody, or antigen-binding fragment thereof, may comprise an LCDR1, an LCDR2, and an LCDR3 comprising the amino acid sequences of SEQ ID NOs: 64, 177, and 290, respectively; and an HCDR1, an HCDR2, and an HCDR3 comprising the amino acid sequences of SEQ ID NOs: 408, 521, and 637, respectively. In some embodiments, the anti-CD3 antibody, or antigen-binding fragment thereof, comprises the VL of antibody ABL-CD3-64. The VL may comprise the amino acid sequence of SEQ ID NO: 751. In some embodiments, the anti-CD3 antibody, or antigen-binding fragment thereof, comprises the VH of antibody ABL-CD3-64. The VH may comprise the amino acid sequence of SEQ ID NO: 978. In some embodiments, the anti-CD3 antibody, or antigen-binding fragment thereof, comprises the VL and VH of antibody ABL-CD3-64. In some embodiments, the anti-CD3 antibody, or antigen-binding fragment thereof, comprises a VL comprising the amino acid sequence of SEQ ID NO: 751 and a VH comprising the amino acid sequence of SEQ ID NO: 978. In some embodiments, the anti-CD3 antibody, or antigen-binding fragment thereof, comprises the VL of antibody ABL-CD3-64A. The VL may comprise the amino acid sequence of SEQ ID NO: 864. In some embodiments, the anti-CD3 antibody, or antigen-binding fragment thereof, comprises the VH of antibody ABL-CD3-64A. The VH may comprise the amino acid sequence of SEQ ID NO: 1091. In some embodiments, the anti-CD3 antibody, or antigen-binding fragment thereof, comprises the VL and VH of antibody ABL-CD3-64A. In some embodiments, the anti-CD3 antibody, or antigen-binding fragment thereof, comprises a VL comprising the amino acid sequence of SEQ ID NO: 864 and a VH comprising the amino acid sequence of SEQ ID NO: 1091. In some embodiments, the anti-CD3 antibody, or antigen-binding fragment thereof, comprises ABL-CD3-64_scFv_VH-VL. In some embodiments, the anti-CD3 antibody, or antigen-binding fragment thereof, comprises, from N-terminus to C-terminus, a VH comprising the amino acid sequence of SEQ ID NO: 978; a linker comprising the amino acid sequence of (Gly-Gly-Gly-Gly-Ser)n (SEQ ID NO: 1146), wherein n is 4; and a VL comprising the amino acid sequence of SEQ ID NO: 751. In some embodiments, the anti-CD3 antibody, or antigen-binding fragment thereof, comprises ABL-CD3-64_scFv_VL-VH. In some embodiments, the anti-CD3 antibody, or antigen-binding fragment thereof, comprises, from N-terminus to C-terminus, a VL comprising the amino acid sequence of SEQ ID NO: 751; a linker comprising the amino acid sequence of (Gly-Gly-Gly-Gly-Ser)n (SEQ ID NO: 1146), wherein n is 4; and a VH comprising the amino acid sequence of SEQ ID NO: 978. In some embodiments, the anti-CD3 antibody, or antigen-binding fragment thereof, comprises ABL-CD3-64A_scFv_VH-VL. In some embodiments, the anti-CD3 antibody, or antigen-binding fragment thereof, comprises the amino acid sequence of SEQ ID NO: 1180. In some embodiments, the anti-CD3 antibody, or antigen-binding fragment thereof, comprises ABL-CD3-64A_scFv_VL-VH. In some embodiments, the anti-CD3 antibody, or antigen-binding fragment thereof, comprises the amino acid sequence of SEQ ID NO: 1221.

In some embodiments, the anti-CD3 antibody, or antigen-binding fragment thereof, comprises the six CDRs of antibody ABL-CD3-65. The anti-CD3 antibody, or antigen-binding fragment thereof, may comprise an LCDR1, an LCDR2, and an LCDR3 comprising the amino acid sequences of SEQ ID NOs: 65, 178, and 291, respectively; and an HCDR1, an HCDR2, and an HCDR3 comprising the amino acid sequences of SEQ ID NOs: 409, 522, and 638, respectively. In some embodiments, the anti-CD3 antibody, or antigen-binding fragment thereof, comprises the six CDRs of antibody ABL-CD3-65A. The anti-CD3 antibody, or antigen-binding fragment thereof, may comprise an LCDR1, an LCDR2, and an LCDR3 comprising the amino acid sequences of SEQ ID NOs: 65, 178, and 291, respectively; and an HCDR1, an HCDR2, and an HCDR3 comprising the amino acid sequences of SEQ ID NOs: 409, 572, and 638, respectively. In some embodiments, the anti-CD3 antibody, or antigen-binding fragment thereof, comprises the VL of antibody ABL-CD3-65. The VL may comprise the amino acid sequence of SEQ ID NO: 752. In some embodiments, the anti-CD3 antibody, or antigen-binding fragment thereof, comprises the VH of antibody ABL-CD3-65. The VH may comprise the amino acid sequence of SEQ ID NO: 979. In some embodiments, the anti-CD3 antibody, or antigen-binding fragment thereof, comprises the VL and VH of antibody ABL-CD3-65. In some embodiments, the anti-CD3 antibody, or antigen-binding fragment thereof, comprises a VL comprising the amino acid sequence of SEQ ID NO: 752 and a VH comprising the amino acid sequence of SEQ ID NO: 979. In some embodiments, the anti-CD3 antibody, or antigen-binding fragment thereof, comprises the VL of antibody ABL-CD3-65A. The VL may comprise the amino acid sequence of SEQ ID NO: 865. In some embodiments, the anti-CD3 antibody, or antigen-binding fragment thereof, comprises the VH of antibody ABL-CD3-65A. The VH may comprise the amino acid sequence of SEQ ID NO: 1092. In some embodiments, the anti-CD3 antibody, or antigen-binding fragment thereof, comprises the VL and VH of antibody ABL-CD3-65A. In some embodiments, the anti-CD3 antibody, or antigen-binding fragment thereof, comprises a VL comprising the amino acid sequence of SEQ ID NO: 865 and a VH comprising the amino acid sequence of SEQ ID NO: 1092. In some embodiments, the anti-CD3 antibody, or antigen-binding fragment thereof, comprises ABL-CD3-65_scFv_VH-VL. In some embodiments, the anti-CD3 antibody, or antigen-binding fragment thereof, comprises, from N-terminus to C-terminus, a VH comprising the amino acid sequence of SEQ ID NO: 979; a linker comprising the amino acid sequence of (Gly-Gly-Gly-Gly-Ser)n (SEQ ID NO: 1146), wherein n is 4; and a VL comprising the amino acid sequence of SEQ ID NO: 752. In some embodiments, the anti-CD3 antibody, or antigen-binding fragment thereof, comprises ABL-CD3-65_scFv_VL-VH. In some embodiments, the anti-CD3 antibody, or antigen-binding fragment thereof, comprises, from N-terminus to C-terminus, a VL comprising the amino acid sequence of SEQ ID NO: 752; a linker comprising the amino acid sequence of (Gly-Gly-Gly-Gly-Ser)n (SEQ ID NO: 1146), wherein n is 4; and a VH comprising the amino acid sequence of SEQ ID NO: 979. In some embodiments, the anti-CD3 antibody, or antigen-binding fragment thereof, comprises ABL-CD3-65A_scFv_VH-VL. In some embodiments, the anti-CD3 antibody, or antigen-binding fragment thereof, comprises the amino acid sequence of SEQ ID NO: 1181. In some embodiments, the anti-CD3 antibody, or antigen-binding fragment thereof, comprises ABL-CD3-65A_scFv_VL-VH. In some embodiments, the anti-CD3 antibody, or antigen-binding fragment thereof, comprises the amino acid sequence of SEQ ID NO: 1222.

In some embodiments, the anti-CD3 antibody, or antigen-binding fragment thereof, comprises the six CDRs of antibody ABL-CD3-66 and ABL-CD3-66A. The anti-CD3 antibody, or antigen-binding fragment thereof, may comprise an LCDR1, an LCDR2, and an LCDR3 comprising the amino acid sequences of SEQ ID NOs: 66, 179, and 292, respectively; and an HCDR1, an HCDR2, and an HCDR3 comprising the amino acid sequences of SEQ ID NOs: 410, 523, and 639, respectively. In some embodiments, the anti-CD3 antibody, or antigen-binding fragment thereof, comprises the VL of antibody ABL-CD3-66. The VL may comprise the amino acid sequence of SEQ ID NO: 753. In some embodiments, the anti-CD3 antibody, or antigen-binding fragment thereof, comprises the VH of antibody ABL-CD3-66. The VH may comprise the amino acid sequence of SEQ ID NO: 980. In some embodiments, the anti-CD3 antibody, or antigen-binding fragment thereof, comprises the VL and VH of antibody ABL-CD3-66. In some embodiments, the anti-CD3 antibody, or antigen-binding fragment thereof, comprises a VL comprising the amino acid sequence of SEQ ID NO: 753 and a VH comprising the amino acid sequence of SEQ ID NO: 980. In some embodiments, the anti-CD3 antibody, or antigen-binding fragment thereof, comprises the VL of antibody ABL-CD3-66A. The VL may comprise the amino acid sequence of SEQ ID NO: 866. In some embodiments, the anti-CD3 antibody, or antigen-binding fragment thereof, comprises the VH of antibody ABL-CD3-66A. The VH may comprise the amino acid sequence of SEQ ID NO: 1093. In some embodiments, the anti-CD3 antibody, or antigen-binding fragment thereof, comprises the VL and VH of antibody ABL-CD3-66A. In some embodiments, the anti-CD3 antibody, or antigen-binding fragment thereof, comprises a VL comprising the amino acid sequence of SEQ ID NO: 866 and a VH comprising the amino acid sequence of SEQ ID NO: 1093. In some embodiments, the anti-CD3 antibody, or antigen-binding fragment thereof, comprises ABL-CD3-66_scFv_VH-VL. In some embodiments, the anti-CD3 antibody, or antigen-binding fragment thereof, comprises, from N-terminus to C-terminus, a VH comprising the amino acid sequence of SEQ ID NO: 980; a linker comprising the amino acid sequence of (Gly-Gly-Gly-Gly-Ser)n (SEQ ID NO: 1146), wherein n is 4; and a VL comprising the amino acid sequence of SEQ ID NO: 753. In some embodiments, the anti-CD3 antibody, or antigen-binding fragment thereof, comprises ABL-CD3-66_scFv_VL-VH. In some embodiments, the anti-CD3 antibody, or antigen-binding fragment thereof, comprises, from N-terminus to C-terminus, a VL comprising the amino acid sequence of SEQ ID NO: 753; a linker comprising the amino acid sequence of (Gly-Gly-Gly-Gly-Ser)n (SEQ ID NO: 1146), wherein n is 4; and a VH comprising the amino acid sequence of SEQ ID NO: 980. In some embodiments, the anti-CD3 antibody, or antigen-binding fragment thereof, comprises ABL-CD3-66A_scFv_VH-VL. In some embodiments, the anti-CD3 antibody, or antigen-binding fragment thereof, comprises the amino acid sequence of SEQ ID NO: 1182. In some embodiments, the anti-CD3 antibody, or antigen-binding fragment thereof, comprises ABL-CD3-66A_scFv_VL-VH. In some embodiments, the anti-CD3 antibody, or antigen-binding fragment thereof, comprises the amino acid sequence of SEQ ID NO: 1223.

In some embodiments, the anti-CD3 antibody, or antigen-binding fragment thereof, comprises the six CDRs of antibody ABL-CD3-67 and ABL-CD3-67A. The anti-CD3 antibody, or antigen-binding fragment thereof, may comprise an LCDR1, an LCDR2, and an LCDR3 comprising the amino acid sequences of SEQ ID NOs: 67, 180, and 293, respectively; and an HCDR1, an HCDR2, and an HCDR3 comprising the amino acid sequences of SEQ ID NOs: 411, 524, and 640, respectively. In some embodiments, the anti-CD3 antibody, or antigen-binding fragment thereof, comprises the VL of antibody ABL-CD3-67. The VL may comprise the amino acid sequence of SEQ ID NO: 754. In some embodiments, the anti-CD3 antibody, or antigen-binding fragment thereof, comprises the VH of antibody ABL-CD3-67. The VH may comprise the amino acid sequence of SEQ ID NO: 981. In some embodiments, the anti-CD3 antibody, or antigen-binding fragment thereof, comprises the VL and VH of antibody ABL-CD3-67. In some embodiments, the anti-CD3 antibody, or antigen-binding fragment thereof, comprises a VL comprising the amino acid sequence of SEQ ID NO: 754 and a VH comprising the amino acid sequence of SEQ ID NO: 981. In some embodiments, the anti-CD3 antibody, or antigen-binding fragment thereof, comprises the VL of antibody ABL-CD3-67A. The VL may comprise the amino acid sequence of SEQ ID NO: 867. In some embodiments, the anti-CD3 antibody, or antigen-binding fragment thereof, comprises the VH of antibody ABL-CD3-67A. The VH may comprise the amino acid sequence of SEQ ID NO: 1094. In some embodiments, the anti-CD3 antibody, or antigen-binding fragment thereof, comprises the VL and VH of antibody ABL-CD3-67A. In some embodiments, the anti-CD3 antibody, or antigen-binding fragment thereof, comprises a VL comprising the amino acid sequence of SEQ ID NO: 867 and a VH comprising the amino acid sequence of SEQ ID NO: 1094. In some embodiments, the anti-CD3 antibody, or antigen-binding fragment thereof, comprises ABL-CD3-67_scFv_VH-VL. In some embodiments, the anti-CD3 antibody, or antigen-binding fragment thereof, comprises, from N-terminus to C-terminus, a VH comprising the amino acid sequence of SEQ ID NO: 981; a linker comprising the amino acid sequence of (Gly-Gly-Gly-Gly-Ser)n (SEQ ID NO: 1146), wherein n is 4; and a VL comprising the amino acid sequence of SEQ ID NO: 754. In some embodiments, the anti-CD3 antibody, or antigen-binding fragment thereof, comprises ABL-CD3-67_scFv_VL-VH. In some embodiments, the anti-CD3 antibody, or antigen-binding fragment thereof, comprises, from N-terminus to C-terminus, a VL comprising the amino acid sequence of SEQ ID NO: 754; a linker comprising the amino acid sequence of (Gly-Gly-Gly-Gly-Ser)n (SEQ ID NO: 1146), wherein n is 4; and a VH comprising the amino acid sequence of SEQ ID NO: 981. In some embodiments, the anti-CD3 antibody, or antigen-binding fragment thereof, comprises ABL-CD3-67A_scFv_VH-VL. In some embodiments, the anti-CD3 antibody, or antigen-binding fragment thereof, comprises, from N-terminus to C-terminus, a VH comprising the amino acid sequence of SEQ ID NO: 1094; a linker comprising the amino acid sequence of (Gly-Gly-Gly-Gly-Ser)n (SEQ ID NO: 1146), wherein n is 4; and a VL comprising the amino acid sequence of SEQ ID NO: 867. In some embodiments, the anti-CD3 antibody, or antigen-binding fragment thereof, comprises ABL-CD3-67A_scFv_VL-VH. In some embodiments, the anti-CD3 antibody, or antigen-binding fragment thereof, comprises, from N-terminus to C-terminus, a VL comprising the amino acid sequence of SEQ ID NO: 867; a linker comprising the amino acid sequence of (Gly-Gly-Gly-Gly-Ser)n (SEQ ID NO: 1146), wherein n is 4; and a VH comprising the amino acid sequence of SEQ ID NO: 1094.

In some embodiments, the anti-CD3 antibody, or antigen-binding fragment thereof, comprises the six CDRs of antibody ABL-CD3-68 and ABL-CD3-68A. The anti-CD3 antibody, or antigen-binding fragment thereof, may comprise an LCDR1, an LCDR2, and an LCDR3 comprising the amino acid sequences of SEQ ID NOs: 68, 181, and 294, respectively; and an HCDR1, an HCDR2, and an HCDR3 comprising the amino acid sequences of SEQ ID NOs: 412, 525, and 641, respectively. In some embodiments, the anti-CD3 antibody, or antigen-binding fragment thereof, comprises the VL of antibody ABL-CD3-68. The VL may comprise the amino acid sequence of SEQ ID NO: 755. In some embodiments, the anti-CD3 antibody, or antigen-binding fragment thereof, comprises the VH of antibody ABL-CD3-68. The VH may comprise the amino acid sequence of SEQ ID NO: 982. In some embodiments, the anti-CD3 antibody, or antigen-binding fragment thereof, comprises the VL and VH of antibody ABL-CD3-68. In some embodiments, the anti-CD3 antibody, or antigen-binding fragment thereof, comprises a VL comprising the amino acid sequence of SEQ ID NO: 755 and a VH comprising the amino acid sequence of SEQ ID NO: 982. In some embodiments, the anti-CD3 antibody, or antigen-binding fragment thereof, comprises the VL of antibody ABL-CD3-68A. The VL may comprise the amino acid sequence of SEQ ID NO: 868. In some embodiments, the anti-CD3 antibody, or antigen-binding fragment thereof, comprises the VH of antibody ABL-CD3-68A. The VH may comprise the amino acid sequence of SEQ ID NO: 1095. In some embodiments, the anti-CD3 antibody, or antigen-binding fragment thereof, comprises the VL and VH of antibody ABL-CD3-68A. In some embodiments, the anti-CD3 antibody, or antigen-binding fragment thereof, comprises a VL comprising the amino acid sequence of SEQ ID NO: 868 and a VH comprising the amino acid sequence of SEQ ID NO: 1095. In some embodiments, the anti-CD3 antibody, or antigen-binding fragment thereof, comprises ABL-CD3-68_scFv_VH-VL. In some embodiments, the anti-CD3 antibody, or antigen-binding fragment thereof, comprises, from N-terminus to C-terminus, a VH comprising the amino acid sequence of SEQ ID NO: 982; a linker comprising the amino acid sequence of (Gly-Gly-Gly-Gly-Ser)n (SEQ ID NO: 1146), wherein n is 4; and a VL comprising the amino acid sequence of SEQ ID NO: 755. In some embodiments, the anti-CD3 antibody, or antigen-binding fragment thereof, comprises ABL-CD3-68_scFv_VL-VH. In some embodiments, the anti-CD3 antibody, or antigen-binding fragment thereof, comprises, from N-terminus to C-terminus, a VL comprising the amino acid sequence of SEQ ID NO: 755; a linker comprising the amino acid sequence of (Gly-Gly-Gly-Gly-Ser)n (SEQ ID NO: 1146), wherein n is 4; and a VH comprising the amino acid sequence of SEQ ID NO: 982. In some embodiments, the anti-CD3 antibody, or antigen-binding fragment thereof, comprises ABL-CD3-68A_scFv_VH-VL. In some embodiments, the anti-CD3 antibody, or antigen-binding fragment thereof, comprises the amino acid sequence of SEQ ID NO: 1183. In some embodiments, the anti-CD3 antibody, or antigen-binding fragment thereof, comprises ABL-CD3-68A_scFv_VL-VH. In some embodiments, the anti-CD3 antibody, or antigen-binding fragment thereof, comprises the amino acid sequence of SEQ ID NO: 1224.

In some embodiments, the anti-CD3 antibody, or antigen-binding fragment thereof, comprises the six CDRs of antibody ABL-CD3-69 and ABL-CD3-69A. The anti-CD3 antibody, or antigen-binding fragment thereof, may comprise an LCDR1, an LCDR2, and an LCDR3 comprising the amino acid sequences of SEQ ID NOs: 69, 182, and 295, respectively; and an HCDR1, an HCDR2, and an HCDR3 comprising the amino acid sequences of SEQ ID NOs: 413, 526, and 642, respectively. In some embodiments, the anti-CD3 antibody, or antigen-binding fragment thereof, comprises the VL of antibody ABL-CD3-69. The VL may comprise the amino acid sequence of SEQ ID NO: 756. In some embodiments, the anti-CD3 antibody, or antigen-binding fragment thereof, comprises the VH of antibody ABL-CD3-69. The VH may comprise the amino acid sequence of SEQ ID NO: 983. In some embodiments, the anti-CD3 antibody, or antigen-binding fragment thereof, comprises the VL and VH of antibody ABL-CD3-69. In some embodiments, the anti-CD3 antibody, or antigen-binding fragment thereof, comprises a VL comprising the amino acid sequence of SEQ ID NO: 756 and a VH comprising the amino acid sequence of SEQ ID NO: 983. In some embodiments, the anti-CD3 antibody, or antigen-binding fragment thereof, comprises the VL of antibody ABL-CD3-69A. The VL may comprise the amino acid sequence of SEQ ID NO: 869. In some embodiments, the anti-CD3 antibody, or antigen-binding fragment thereof, comprises the VH of antibody ABL-CD3-69A. The VH may comprise the amino acid sequence of SEQ ID NO: 1096. In some embodiments, the anti-CD3 antibody, or antigen-binding fragment thereof, comprises the VL and VH of antibody ABL-CD3-69A. In some embodiments, the anti-CD3 antibody, or antigen-binding fragment thereof, comprises a VL comprising the amino acid sequence of SEQ ID NO: 869 and a VH comprising the amino acid sequence of SEQ ID NO: 1096. In some embodiments, the anti-CD3 antibody, or antigen-binding fragment thereof, comprises ABL-CD3-69_scFv_VH-VL. In some embodiments, the anti-CD3 antibody, or antigen-binding fragment thereof, comprises, from N-terminus to C-terminus, a VH comprising the amino acid sequence of SEQ ID NO: 983; a linker comprising the amino acid sequence of (Gly-Gly-Gly-Gly-Ser)n (SEQ ID NO: 1146), wherein n is 4; and a VL comprising the amino acid sequence of SEQ ID NO: 756. In some embodiments, the anti-CD3 antibody, or antigen-binding fragment thereof, comprises ABL-CD3-69_scFv_VL-VH. In some embodiments, the anti-CD3 antibody, or antigen-binding fragment thereof, comprises, from N-terminus to C-terminus, a VL comprising the amino acid sequence of SEQ ID NO: 756; a linker comprising the amino acid sequence of (Gly-Gly-Gly-Gly-Ser)n (SEQ ID NO: 1146), wherein n is 4; and a VH comprising the amino acid sequence of SEQ ID NO: 983. In some embodiments, the anti-CD3 antibody, or antigen-binding fragment thereof, comprises ABL-CD3-69A_scFv_VH-VL. In some embodiments, the anti-CD3 antibody, or antigen-binding fragment thereof, comprises, from N-terminus to C-terminus, a VH comprising the amino acid sequence of SEQ ID NO: 1096; a linker comprising the amino acid sequence of (Gly-Gly-Gly-Gly-Ser)n (SEQ ID NO: 1146), wherein n is 4; and a VL comprising the amino acid sequence of SEQ ID NO: 869. In some embodiments, the anti-CD3 antibody, or antigen-binding fragment thereof, comprises ABL-CD3-69A_scFv_VL-VH. In some embodiments, the anti-CD3 antibody, or antigen-binding fragment thereof, comprises, from N-terminus to C-terminus, a VL comprising the amino acid sequence of SEQ ID NO: 869; a linker comprising the amino acid sequence of (Gly-Gly-Gly-Gly-Ser)n (SEQ ID NO: 1146), wherein n is 4; and a VH comprising the amino acid sequence of SEQ ID NO: 1096.

In some embodiments, the anti-CD3 antibody, or antigen-binding fragment thereof, comprises the six CDRs of antibody ABL-CD3-70 and ABL-CD3-70A. The anti-CD3 antibody, or antigen-binding fragment thereof, may comprise an LCDR1, an LCDR2, and an LCDR3 comprising the amino acid sequences of SEQ ID NOs: 70, 183, and 296, respectively; and an HCDR1, an HCDR2, and an HCDR3 comprising the amino acid sequences of SEQ ID NOs: 414, 527, and 643, respectively. In some embodiments, the anti-CD3 antibody, or antigen-binding fragment thereof, comprises the VL of antibody ABL-CD3-70. The VL may comprise the amino acid sequence of SEQ ID NO: 757. In some embodiments, the anti-CD3 antibody, or antigen-binding fragment thereof, comprises the VH of antibody ABL-CD3-70. The VH may comprise the amino acid sequence of SEQ ID NO: 984. In some embodiments, the anti-CD3 antibody, or antigen-binding fragment thereof, comprises the VL and VH of antibody ABL-CD3-70. In some embodiments, the anti-CD3 antibody, or antigen-binding fragment thereof, comprises a VL comprising the amino acid sequence of SEQ ID NO: 757 and a VH comprising the amino acid sequence of SEQ ID NO: 984. In some embodiments, the anti-CD3 antibody, or antigen-binding fragment thereof, comprises the VL of antibody ABL-CD3-70A. The VL may comprise the amino acid sequence of SEQ ID NO: 870. In some embodiments, the anti-CD3 antibody, or antigen-binding fragment thereof, comprises the VH of antibody ABL-CD3-70A. The VH may comprise the amino acid sequence of SEQ ID NO: 1097. In some embodiments, the anti-CD3 antibody, or antigen-binding fragment thereof, comprises the VL and VH of antibody ABL-CD3-70A. In some embodiments, the anti-CD3 antibody, or antigen-binding fragment thereof, comprises a VL comprising the amino acid sequence of SEQ ID NO: 870 and a VH comprising the amino acid sequence of SEQ ID NO: 1097. In some embodiments, the anti-CD3 antibody, or antigen-binding fragment thereof, comprises ABL-CD3-70_scFv_VH-VL. In some embodiments, the anti-CD3 antibody, or antigen-binding fragment thereof, comprises, from N-terminus to C-terminus, a VH comprising the amino acid sequence of SEQ ID NO: 984; a linker comprising the amino acid sequence of (Gly-Gly-Gly-Gly-Ser)n (SEQ ID NO: 1146), wherein n is 4; and a VL comprising the amino acid sequence of SEQ ID NO: 757. In some embodiments, the anti-CD3 antibody, or antigen-binding fragment thereof, comprises ABL-CD3-70_scFv_VL-VH. In some embodiments, the anti-CD3 antibody, or antigen-binding fragment thereof, comprises, from N-terminus to C-terminus, a VL comprising the amino acid sequence of SEQ ID NO: 757; a linker comprising the amino acid sequence of (Gly-Gly-Gly-Gly-Ser)n (SEQ ID NO: 1146), wherein n is 4; and a VH comprising the amino acid sequence of SEQ ID NO: 984. In some embodiments, the anti-CD3 antibody, or antigen-binding fragment thereof, comprises ABL-CD3-70A_scFv_VH-VL. In some embodiments, the anti-CD3 antibody, or antigen-binding fragment thereof, comprises, from N-terminus to C-terminus, a VH comprising the amino acid sequence of SEQ ID NO: 1097; a linker comprising the amino acid sequence of (Gly-Gly-Gly-Gly-Ser)n (SEQ ID NO: 1146), wherein n is 4; and a VL comprising the amino acid sequence of SEQ ID NO: 870. In some embodiments, the anti-CD3 antibody, or antigen-binding fragment thereof, comprises ABL-CD3-70A_scFv_VL-VH. In some embodiments, the anti-CD3 antibody, or antigen-binding fragment thereof, comprises, from N-terminus to C-terminus, a VL comprising the amino acid sequence of SEQ ID NO: 870; a linker comprising the amino acid sequence of (Gly-Gly-Gly-Gly-Ser)n (SEQ ID NO: 1146), wherein n is 4; and a VH comprising the amino acid sequence of SEQ ID NO: 1097.

In some embodiments, the anti-CD3 antibody, or antigen-binding fragment thereof, comprises the six CDRs of antibody ABL-CD3-71 and ABL-CD3-71A. The anti-CD3 antibody, or antigen-binding fragment thereof, may comprise an LCDR1, an LCDR2, and an LCDR3 comprising the amino acid sequences of SEQ ID NOs: 71, 184, and 297, respectively; and an HCDR1, an HCDR2, and an HCDR3 comprising the amino acid sequences of SEQ ID NOs: 415, 528, and 644, respectively. In some embodiments, the anti-CD3 antibody, or antigen-binding fragment thereof, comprises the VL of antibody ABL-CD3-71. The VL may comprise the amino acid sequence of SEQ ID NO: 758. In some embodiments, the anti-CD3 antibody, or antigen-binding fragment thereof, comprises the VH of antibody ABL-CD3-71. The VH may comprise the amino acid sequence of SEQ ID NO: 985. In some embodiments, the anti-CD3 antibody, or antigen-binding fragment thereof, comprises the VL and VH of antibody ABL-CD3-71. In some embodiments, the anti-CD3 antibody, or antigen-binding fragment thereof, comprises a VL comprising the amino acid sequence of SEQ ID NO: 758 and a VH comprising the amino acid sequence of SEQ ID NO: 985. In some embodiments, the anti-CD3 antibody, or antigen-binding fragment thereof, comprises the VL of antibody ABL-CD3-71A. The VL may comprise the amino acid sequence of SEQ ID NO: 871. In some embodiments, the anti-CD3 antibody, or antigen-binding fragment thereof, comprises the VH of antibody ABL-CD3-71A. The VH may comprise the amino acid sequence of SEQ ID NO: 1098. In some embodiments, the anti-CD3 antibody, or antigen-binding fragment thereof, comprises the VL and VH of antibody ABL-CD3-71A. In some embodiments, the anti-CD3 antibody, or antigen-binding fragment thereof, comprises a VL comprising the amino acid sequence of SEQ ID NO: 871 and a VH comprising the amino acid sequence of SEQ ID NO: 1098. In some embodiments, the anti-CD3 antibody, or antigen-binding fragment thereof, comprises ABL-CD3-71_scFv_VH-VL. In some embodiments, the anti-CD3 antibody, or antigen-binding fragment thereof, comprises, from N-terminus to C-terminus, a VH comprising the amino acid sequence of SEQ ID NO: 985; a linker comprising the amino acid sequence of (Gly-Gly-Gly-Gly-Ser)n (SEQ ID NO: 1146), wherein n is 4; and a VL comprising the amino acid sequence of SEQ ID NO: 758. In some embodiments, the anti-CD3 antibody, or antigen-binding fragment thereof, comprises ABL-CD3-71_scFv_VL-VH. In some embodiments, the anti-CD3 antibody, or antigen-binding fragment thereof, comprises, from N-terminus to C-terminus, a VL comprising the amino acid sequence of SEQ ID NO: 758; a linker comprising the amino acid sequence of (Gly-Gly-Gly-Gly-Ser)n (SEQ ID NO: 1146), wherein n is 4; and a VH comprising the amino acid sequence of SEQ ID NO: 985. In some embodiments, the anti-CD3 antibody, or antigen-binding fragment thereof, comprises ABL-CD3-71A_scFv_VH-VL. In some embodiments, the anti-CD3 antibody, or antigen-binding fragment thereof, comprises the amino acid sequence of SEQ ID NO: 1184. In some embodiments, the anti-CD3 antibody, or antigen-binding fragment thereof, comprises ABL-CD3-71A_scFv_VL-VH. In some embodiments, the anti-CD3 antibody, or antigen-binding fragment thereof, comprises the amino acid sequence of SEQ ID NO: 1225.

In some embodiments, the anti-CD3 antibody, or antigen-binding fragment thereof, comprises the six CDRs of antibody ABL-CD3-72 and ABL-CD3-72A. The anti-CD3 antibody, or antigen-binding fragment thereof, may comprise an LCDR1, an LCDR2, and an LCDR3 comprising the amino acid sequences of SEQ ID NOs: 72, 185, and 298, respectively; and an HCDR1, an HCDR2, and an HCDR3 comprising the amino acid sequences of SEQ ID NOs: 416, 529, and 645, respectively. In some embodiments, the anti-CD3 antibody, or antigen-binding fragment thereof, comprises the VL of antibody ABL-CD3-72. The VL may comprise the amino acid sequence of SEQ ID NO: 759. In some embodiments, the anti-CD3 antibody, or antigen-binding fragment thereof, comprises the VH of antibody ABL-CD3-72. The VH may comprise the amino acid sequence of SEQ ID NO: 986. In some embodiments, the anti-CD3 antibody, or antigen-binding fragment thereof, comprises the VL and VH of antibody ABL-CD3-72. In some embodiments, the anti-CD3 antibody, or antigen-binding fragment thereof, comprises a VL comprising the amino acid sequence of SEQ ID NO: 759 and a VH comprising the amino acid sequence of SEQ ID NO: 986. In some embodiments, the anti-CD3 antibody, or antigen-binding fragment thereof, comprises the VL of antibody ABL-CD3-72A. The VL may comprise the amino acid sequence of SEQ ID NO: 872. In some embodiments, the anti-CD3 antibody, or antigen-binding fragment thereof, comprises the VH of antibody ABL-CD3-72A. The VH may comprise the amino acid sequence of SEQ ID NO: 1099. In some embodiments, the anti-CD3 antibody, or antigen-binding fragment thereof, comprises the VL and VH of antibody ABL-CD3-72A. In some embodiments, the anti-CD3 antibody, or antigen-binding fragment thereof, comprises a VL comprising the amino acid sequence of SEQ ID NO: 872 and a VH comprising the amino acid sequence of SEQ ID NO: 1099. In some embodiments, the anti-CD3 antibody, or antigen-binding fragment thereof, comprises ABL-CD3-72_scFv_VH-VL. In some embodiments, the anti-CD3 antibody, or antigen-binding fragment thereof, comprises, from N-terminus to C-terminus, a VH comprising the amino acid sequence of SEQ ID NO: 986; a linker comprising the amino acid sequence of (Gly-Gly-Gly-Gly-Ser)n (SEQ ID NO: 1146), wherein n is 4; and a VL comprising the amino acid sequence of SEQ ID NO: 759. In some embodiments, the anti-CD3 antibody, or antigen-binding fragment thereof, comprises ABL-CD3-72_scFv_VL-VH. In some embodiments, the anti-CD3 antibody, or antigen-binding fragment thereof, comprises, from N-terminus to C-terminus, a VL comprising the amino acid sequence of SEQ ID NO: 759; a linker comprising the amino acid sequence of (Gly-Gly-Gly-Gly-Ser)n (SEQ ID NO: 1146), wherein n is 4; and a VH comprising the amino acid sequence of SEQ ID NO: 986. In some embodiments, the anti-CD3 antibody, or antigen-binding fragment thereof, comprises ABL-CD3-72A_scFv_VH-VL. In some embodiments, the anti-CD3 antibody, or antigen-binding fragment thereof, comprises the amino acid sequence of SEQ ID NO: 1185. In some embodiments, the anti-CD3 antibody, or antigen-binding fragment thereof, comprises ABL-CD3-72A_scFv_VL-VH. In some embodiments, the anti-CD3 antibody, or antigen-binding fragment thereof, comprises the amino acid sequence of SEQ ID NO: 1226.

In some embodiments, the anti-CD3 antibody, or antigen-binding fragment thereof, comprises the six CDRs of antibody ABL-CD3-73 and ABL-CD3-73A. The anti-CD3 antibody, or antigen-binding fragment thereof, may comprise an LCDR1, an LCDR2, and an LCDR3 comprising the amino acid sequences of SEQ ID NOs: 73, 186, and 299, respectively; and an HCDR1, an HCDR2, and an HCDR3 comprising the amino acid sequences of SEQ ID NOs: 417, 530, and 646, respectively. In some embodiments, the anti-CD3 antibody, or antigen-binding fragment thereof, comprises the VL of antibody ABL-CD3-73. The VL may comprise the amino acid sequence of SEQ ID NO: 760. In some embodiments, the anti-CD3 antibody, or antigen-binding fragment thereof, comprises the VH of antibody ABL-CD3-73. The VH may comprise the amino acid sequence of SEQ ID NO: 987. In some embodiments, the anti-CD3 antibody, or antigen-binding fragment thereof, comprises the VL and VH of antibody ABL-CD3-73. In some embodiments, the anti-CD3 antibody, or antigen-binding fragment thereof, comprises a VL comprising the amino acid sequence of SEQ ID NO: 760 and a VH comprising the amino acid sequence of SEQ ID NO: 987. In some embodiments, the anti-CD3 antibody, or antigen-binding fragment thereof, comprises the VL of antibody ABL-CD3-73A. The VL may comprise the amino acid sequence of SEQ ID NO: 873. In some embodiments, the anti-CD3 antibody, or antigen-binding fragment thereof, comprises the VH of antibody ABL-CD3-73A. The VH may comprise the amino acid sequence of SEQ ID NO: 1100. In some embodiments, the anti-CD3 antibody, or antigen-binding fragment thereof, comprises the VL and VH of antibody ABL-CD3-73A. In some embodiments, the anti-CD3 antibody, or antigen-binding fragment thereof, comprises a VL comprising the amino acid sequence of SEQ ID NO: 873 and a VH comprising the amino acid sequence of SEQ ID NO: 1100. In some embodiments, the anti-CD3 antibody, or antigen-binding fragment thereof, comprises ABL-CD3-73_scFv_VH-VL. In some embodiments, the anti-CD3 antibody, or antigen-binding fragment thereof, comprises, from N-terminus to C-terminus, a VH comprising the amino acid sequence of SEQ ID NO: 987; a linker comprising the amino acid sequence of (Gly-Gly-Gly-Gly-Ser)n (SEQ ID NO: 1146), wherein n is 4; and a VL comprising the amino acid sequence of SEQ ID NO: 760. In some embodiments, the anti-CD3 antibody, or antigen-binding fragment thereof, comprises ABL-CD3-73_scFv_VL-VH. In some embodiments, the anti-CD3 antibody, or antigen-binding fragment thereof, comprises, from N-terminus to C-terminus, a VL comprising the amino acid sequence of SEQ ID NO: 760; a linker comprising the amino acid sequence of (Gly-Gly-Gly-Gly-Ser)n (SEQ ID NO: 1146), wherein n is 4; and a VH comprising the amino acid sequence of SEQ ID NO: 987. In some embodiments, the anti-CD3 antibody, or antigen-binding fragment thereof, comprises ABL-CD3-73A_scFv_VH-VL. In some embodiments, the anti-CD3 antibody, or antigen-binding fragment thereof, comprises, from N-terminus to C-terminus, a VH comprising the amino acid sequence of SEQ ID NO: 1100; a linker comprising the amino acid sequence of (Gly-Gly-Gly-Gly-Ser)n (SEQ ID NO: 1146), wherein n is 4; and a VL comprising the amino acid sequence of SEQ ID NO: 873. In some embodiments, the anti-CD3 antibody, or antigen-binding fragment thereof, comprises ABL-CD3-73A_scFv_VL-VH. In some embodiments, the anti-CD3 antibody, or antigen-binding fragment thereof, comprises, from N-terminus to C-terminus, a VL comprising the amino acid sequence of SEQ ID NO: 873; a linker comprising the amino acid sequence of (Gly-Gly-Gly-Gly-Ser)n (SEQ ID NO: 1146), wherein n is 4; and a VH comprising the amino acid sequence of SEQ ID NO: 1100.

In some embodiments, the anti-CD3 antibody, or antigen-binding fragment thereof, comprises the six CDRs of antibody ABL-CD3-74 and ABL-CD3-74A. The anti-CD3 antibody, or antigen-binding fragment thereof, may comprise an LCDR1, an LCDR2, and an LCDR3 comprising the amino acid sequences of SEQ ID NOs: 74, 187, and 300, respectively; and an HCDR1, an HCDR2, and an HCDR3 comprising the amino acid sequences of SEQ ID NOs: 418, 531, and 647, respectively. In some embodiments, the anti-CD3 antibody, or antigen-binding fragment thereof, comprises the VL of antibody ABL-CD3-74. The VL may comprise the amino acid sequence of SEQ ID NO: 761. In some embodiments, the anti-CD3 antibody, or antigen-binding fragment thereof, comprises the VH of antibody ABL-CD3-74. The VH may comprise the amino acid sequence of SEQ ID NO: 988. In some embodiments, the anti-CD3 antibody, or antigen-binding fragment thereof, comprises the VL and VH of antibody ABL-CD3-74. In some embodiments, the anti-CD3 antibody, or antigen-binding fragment thereof, comprises a VL comprising the amino acid sequence of SEQ ID NO: 761 and a VH comprising the amino acid sequence of SEQ ID NO: 988. In some embodiments, the anti-CD3 antibody, or antigen-binding fragment thereof, comprises the VL of antibody ABL-CD3-74A. The VL may comprise the amino acid sequence of SEQ ID NO: 874. In some embodiments, the anti-CD3 antibody, or antigen-binding fragment thereof, comprises the VH of antibody ABL-CD3-74A. The VH may comprise the amino acid sequence of SEQ ID NO: 1101. In some embodiments, the anti-CD3 antibody, or antigen-binding fragment thereof, comprises the VL and VH of antibody ABL-CD3-74A. In some embodiments, the anti-CD3 antibody, or antigen-binding fragment thereof, comprises a VL comprising the amino acid sequence of SEQ ID NO: 874 and a VH comprising the amino acid sequence of SEQ ID NO: 1101. In some embodiments, the anti-CD3 antibody, or antigen-binding fragment thereof, comprises ABL-CD3-74_scFv_VH-VL. In some embodiments, the anti-CD3 antibody, or antigen-binding fragment thereof, comprises, from N-terminus to C-terminus, a VH comprising the amino acid sequence of SEQ ID NO: 988; a linker comprising the amino acid sequence of (Gly-Gly-Gly-Gly-Ser)n (SEQ ID NO: 1146), wherein n is 4; and a VL comprising the amino acid sequence of SEQ ID NO: 761. In some embodiments, the anti-CD3 antibody, or antigen-binding fragment thereof, comprises ABL-CD3-74_scFv_VL-VH. In some embodiments, the anti-CD3 antibody, or antigen-binding fragment thereof, comprises, from N-terminus to C-terminus, a VL comprising the amino acid sequence of SEQ ID NO: 761; a linker comprising the amino acid sequence of (Gly-Gly-Gly-Gly-Ser)n (SEQ ID NO: 1146), wherein n is 4; and a VH comprising the amino acid sequence of SEQ ID NO: 988. In some embodiments, the anti-CD3 antibody, or antigen-binding fragment thereof, comprises ABL-CD3-74A_scFv_VH-VL. In some embodiments, the anti-CD3 antibody, or antigen-binding fragment thereof, comprises the amino acid sequence of SEQ ID NO: 1186. In some embodiments, the anti-CD3 antibody, or antigen-binding fragment thereof, comprises ABL-CD3-74A_scFv_VL-VH. In some embodiments, the anti-CD3 antibody, or antigen-binding fragment thereof, comprises the amino acid sequence of SEQ ID NO: 1227.

In some embodiments, the anti-CD3 antibody, or antigen-binding fragment thereof, comprises the six CDRs of antibody ABL-CD3-75 and ABL-CD3-75A. The anti-CD3 antibody, or antigen-binding fragment thereof, may comprise an LCDR1, an LCDR2, and an LCDR3 comprising the amino acid sequences of SEQ ID NOs: 75, 188, and 301, respectively; and an HCDR1, an HCDR2, and an HCDR3 comprising the amino acid sequences of SEQ ID NOs: 419, 532, and 648, respectively. In some embodiments, the anti-CD3 antibody, or antigen-binding fragment thereof, comprises the VL of antibody ABL-CD3-75. The VL may comprise the amino acid sequence of SEQ ID NO: 762. In some embodiments, the anti-CD3 antibody, or antigen-binding fragment thereof, comprises the VH of antibody ABL-CD3-75. The VH may comprise the amino acid sequence of SEQ ID NO: 989. In some embodiments, the anti-CD3 antibody, or antigen-binding fragment thereof, comprises the VL and VH of antibody ABL-CD3-75. In some embodiments, the anti-CD3 antibody, or antigen-binding fragment thereof, comprises a VL comprising the amino acid sequence of SEQ ID NO: 762 and a VH comprising the amino acid sequence of SEQ ID NO: 989. In some embodiments, the anti-CD3 antibody, or antigen-binding fragment thereof, comprises the VL of antibody ABL-CD3-75A. The VL may comprise the amino acid sequence of SEQ ID NO: 875. In some embodiments, the anti-CD3 antibody, or antigen-binding fragment thereof, comprises the VH of antibody ABL-CD3-75A. The VH may comprise the amino acid sequence of SEQ ID NO: 1102. In some embodiments, the anti-CD3 antibody, or antigen-binding fragment thereof, comprises the VL and VH of antibody ABL-CD3-75A. In some embodiments, the anti-CD3 antibody, or antigen-binding fragment thereof, comprises a VL comprising the amino acid sequence of SEQ ID NO: 875 and a VH comprising the amino acid sequence of SEQ ID NO: 1102. In some embodiments, the anti-CD3 antibody, or antigen-binding fragment thereof, comprises ABL-CD3-75_scFv_VH-VL. In some embodiments, the anti-CD3 antibody, or antigen-binding fragment thereof, comprises, from N-terminus to C-terminus, a VH comprising the amino acid sequence of SEQ ID NO: 989; a linker comprising the amino acid sequence of (Gly-Gly-Gly-Gly-Ser)n (SEQ ID NO: 1146), wherein n is 4; and a VL comprising the amino acid sequence of SEQ ID NO: 762. In some embodiments, the anti-CD3 antibody, or antigen-binding fragment thereof, comprises ABL-CD3-75_scFv_VL-VH. In some embodiments, the anti-CD3 antibody, or antigen-binding fragment thereof, comprises, from N-terminus to C-terminus, a VL comprising the amino acid sequence of SEQ ID NO: 762; a linker comprising the amino acid sequence of (Gly-Gly-Gly-Gly-Ser)n (SEQ ID NO: 1146), wherein n is 4; and a VH comprising the amino acid sequence of SEQ ID NO: 989. In some embodiments, the anti-CD3 antibody, or antigen-binding fragment thereof, comprises ABL-CD3-75A_scFv_VH-VL. In some embodiments, the anti-CD3 antibody, or antigen-binding fragment thereof, comprises, from N-terminus to C-terminus, a VH comprising the amino acid sequence of SEQ ID NO: 1102; a linker comprising the amino acid sequence of (Gly-Gly-Gly-Gly-Ser)n (SEQ ID NO: 1146), wherein n is 4; and a VL comprising the amino acid sequence of SEQ ID NO: 875. In some embodiments, the anti-CD3 antibody, or antigen-binding fragment thereof, comprises ABL-CD3-75A_scFv_VL-VH. In some embodiments, the anti-CD3 antibody, or antigen-binding fragment thereof, comprises, from N-terminus to C-terminus, a VL comprising the amino acid sequence of SEQ ID NO: 875; a linker comprising the amino acid sequence of (Gly-Gly-Gly-Gly-Ser)n (SEQ ID NO: 1146), wherein n is 4; and a VH comprising the amino acid sequence of SEQ ID NO: 1102.

In some embodiments, the anti-CD3 antibody, or antigen-binding fragment thereof, comprises the six CDRs of antibody ABL-CD3-76 and ABL-CD3-76A. The anti-CD3 antibody, or antigen-binding fragment thereof, may comprise an LCDR1, an LCDR2, and an LCDR3 comprising the amino acid sequences of SEQ ID NOs: 76, 189, and 302, respectively; and an HCDR1, an HCDR2, and an HCDR3 comprising the amino acid sequences of SEQ ID NOs: 420, 533, and 649, respectively. In some embodiments, the anti-CD3 antibody, or antigen-binding fragment thereof, comprises the VL of antibody ABL-CD3-76. The VL may comprise the amino acid sequence of SEQ ID NO: 763. In some embodiments, the anti-CD3 antibody, or antigen-binding fragment thereof, comprises the VH of antibody ABL-CD3-76. The VH may comprise the amino acid sequence of SEQ ID NO: 990. In some embodiments, the anti-CD3 antibody, or antigen-binding fragment thereof, comprises the VL and VH of antibody ABL-CD3-76. In some embodiments, the anti-CD3 antibody, or antigen-binding fragment thereof, comprises a VL comprising the amino acid sequence of SEQ ID NO: 763 and a VH comprising the amino acid sequence of SEQ ID NO: 990. In some embodiments, the anti-CD3 antibody, or antigen-binding fragment thereof, comprises the VL of antibody ABL-CD3-76A. The VL may comprise the amino acid sequence of SEQ ID NO: 876. In some embodiments, the anti-CD3 antibody, or antigen-binding fragment thereof, comprises the VH of antibody ABL-CD3-76A. The VH may comprise the amino acid sequence of SEQ ID NO: 1103. In some embodiments, the anti-CD3 antibody, or antigen-binding fragment thereof, comprises the VL and VH of antibody ABL-CD3-76A. In some embodiments, the anti-CD3 antibody, or antigen-binding fragment thereof, comprises a VL comprising the amino acid sequence of SEQ ID NO: 876 and a VH comprising the amino acid sequence of SEQ ID NO: 1103. In some embodiments, the anti-CD3 antibody, or antigen-binding fragment thereof, comprises ABL-CD3-76_scFv_VH-VL. In some embodiments, the anti-CD3 antibody, or antigen-binding fragment thereof, comprises, from N-terminus to C-terminus, a VH comprising the amino acid sequence of SEQ ID NO: 990; a linker comprising the amino acid sequence of (Gly-Gly-Gly-Gly-Ser)n (SEQ ID NO: 1146), wherein n is 4; and a VL comprising the amino acid sequence of SEQ ID NO: 763. In some embodiments, the anti-CD3 antibody, or antigen-binding fragment thereof, comprises ABL-CD3-76_scFv_VL-VH. In some embodiments, the anti-CD3 antibody, or antigen-binding fragment thereof, comprises, from N-terminus to C-terminus, a VL comprising the amino acid sequence of SEQ ID NO: 763; a linker comprising the amino acid sequence of (Gly-Gly-Gly-Gly-Ser)n (SEQ ID NO: 1146), wherein n is 4; and a VH comprising the amino acid sequence of SEQ ID NO: 990. In some embodiments, the anti-CD3 antibody, or antigen-binding fragment thereof, comprises ABL-CD3-76A_scFv_VH-VL. In some embodiments, the anti-CD3 antibody, or antigen-binding fragment thereof, comprises the amino acid sequence of SEQ ID NO: 1187. In some embodiments, the anti-CD3 antibody, or antigen-binding fragment thereof, comprises ABL-CD3-76A_scFv_VL-VH. In some embodiments, the anti-CD3 antibody, or antigen-binding fragment thereof, comprises the amino acid sequence of SEQ ID NO: 1228.

In some embodiments, the anti-CD3 antibody, or antigen-binding fragment thereof, comprises the six CDRs of antibody ABL-CD3-77 and ABL-CD3-77A. The anti-CD3 antibody, or antigen-binding fragment thereof, may comprise an LCDR1, an LCDR2, and an LCDR3 comprising the amino acid sequences of SEQ ID NOs: 77, 190, and 303, respectively; and an HCDR1, an HCDR2, and an HCDR3 comprising the amino acid sequences of SEQ ID NOs: 421, 534, and 650, respectively. In some embodiments, the anti-CD3 antibody, or antigen-binding fragment thereof, comprises the VL of antibody ABL-CD3-77. The VL may comprise the amino acid sequence of SEQ ID NO: 764. In some embodiments, the anti-CD3 antibody, or antigen-binding fragment thereof, comprises the VH of antibody ABL-CD3-77. The VH may comprise the amino acid sequence of SEQ ID NO: 991. In some embodiments, the anti-CD3 antibody, or antigen-binding fragment thereof, comprises the VL and VH of antibody ABL-CD3-77. In some embodiments, the anti-CD3 antibody, or antigen-binding fragment thereof, comprises a VL comprising the amino acid sequence of SEQ ID NO: 764 and a VH comprising the amino acid sequence of SEQ ID NO: 991. In some embodiments, the anti-CD3 antibody, or antigen-binding fragment thereof, comprises the VL of antibody ABL-CD3-77A. The VL may comprise the amino acid sequence of SEQ ID NO: 877. In some embodiments, the anti-CD3 antibody, or antigen-binding fragment thereof, comprises the VH of antibody ABL-CD3-77A. The VH may comprise the amino acid sequence of SEQ ID NO: 1104. In some embodiments, the anti-CD3 antibody, or antigen-binding fragment thereof, comprises the VL and VH of antibody ABL-CD3-77A. In some embodiments, the anti-CD3 antibody, or antigen-binding fragment thereof, comprises a VL comprising the amino acid sequence of SEQ ID NO: 877 and a VH comprising the amino acid sequence of SEQ ID NO: 1104. In some embodiments, the anti-CD3 antibody, or antigen-binding fragment thereof, comprises ABL-CD3-77_scFv_VH-VL. In some embodiments, the anti-CD3 antibody, or antigen-binding fragment thereof, comprises, from N-terminus to C-terminus, a VH comprising the amino acid sequence of SEQ ID NO: 991; a linker comprising the amino acid sequence of (Gly-Gly-Gly-Gly-Ser)n (SEQ ID NO: 1146), wherein n is 4; and a VL comprising the amino acid sequence of SEQ ID NO: 764. In some embodiments, the anti-CD3 antibody, or antigen-binding fragment thereof, comprises ABL-CD3-77_scFv_VL-VH. In some embodiments, the anti-CD3 antibody, or antigen-binding fragment thereof, comprises, from N-terminus to C-terminus, a VL comprising the amino acid sequence of SEQ ID NO: 764; a linker comprising the amino acid sequence of (Gly-Gly-Gly-Gly-Ser)n (SEQ ID NO: 1146), wherein n is 4; and a VH comprising the amino acid sequence of SEQ ID NO: 991. In some embodiments, the anti-CD3 antibody, or antigen-binding fragment thereof, comprises ABL-CD3-77A_scFv_VH-VL. In some embodiments, the anti-CD3 antibody, or antigen-binding fragment thereof, comprises the amino acid sequence of SEQ ID NO: 1188. In some embodiments, the anti-CD3 antibody, or antigen-binding fragment thereof, comprises ABL-CD3-77A_scFv_VL-VH. In some embodiments, the anti-CD3 antibody, or antigen-binding fragment thereof, comprises the amino acid sequence of SEQ ID NO: 1229.

In some embodiments, the anti-CD3 antibody, or antigen-binding fragment thereof, comprises the six CDRs of antibody ABL-CD3-78 and ABL-CD3-78A. The anti-CD3 antibody, or antigen-binding fragment thereof, may comprise an LCDR1, an LCDR2, and an LCDR3 comprising the amino acid sequences of SEQ ID NOs: 78, 191, and 304, respectively; and an HCDR1, an HCDR2, and an HCDR3 comprising the amino acid sequences of SEQ ID NOs: 422, 535, and 651, respectively. In some embodiments, the anti-CD3 antibody, or antigen-binding fragment thereof, comprises the VL of antibody ABL-CD3-78. The VL may comprise the amino acid sequence of SEQ ID NO: 765. In some embodiments, the anti-CD3 antibody, or antigen-binding fragment thereof, comprises the VH of antibody ABL-CD3-78. The VH may comprise the amino acid sequence of SEQ ID NO: 992. In some embodiments, the anti-CD3 antibody, or antigen-binding fragment thereof, comprises the VL and VH of antibody ABL-CD3-78. In some embodiments, the anti-CD3 antibody, or antigen-binding fragment thereof, comprises a VL comprising the amino acid sequence of SEQ ID NO: 765 and a VH comprising the amino acid sequence of SEQ ID NO: 992. In some embodiments, the anti-CD3 antibody, or antigen-binding fragment thereof, comprises the VL of antibody ABL-CD3-78A. The VL may comprise the amino acid sequence of SEQ ID NO: 878. In some embodiments, the anti-CD3 antibody, or antigen-binding fragment thereof, comprises the VH of antibody ABL-CD3-78A. The VH may comprise the amino acid sequence of SEQ ID NO: 1105. In some embodiments, the anti-CD3 antibody, or antigen-binding fragment thereof, comprises the VL and VH of antibody ABL-CD3-78A. In some embodiments, the anti-CD3 antibody, or antigen-binding fragment thereof, comprises a VL comprising the amino acid sequence of SEQ ID NO: 878 and a VH comprising the amino acid sequence of SEQ ID NO: 1105. In some embodiments, the anti-CD3 antibody, or antigen-binding fragment thereof, comprises ABL-CD3-78_scFv_VH-VL. In some embodiments, the anti-CD3 antibody, or antigen-binding fragment thereof, comprises, from N-terminus to C-terminus, a VH comprising the amino acid sequence of SEQ ID NO: 992; a linker comprising the amino acid sequence of (Gly-Gly-Gly-Gly-Ser)n (SEQ ID NO: 1146), wherein n is 4; and a VL comprising the amino acid sequence of SEQ ID NO: 765. In some embodiments, the anti-CD3 antibody, or antigen-binding fragment thereof, comprises ABL-CD3-78_scFv_VL-VH. In some embodiments, the anti-CD3 antibody, or antigen-binding fragment thereof, comprises, from N-terminus to C-terminus, a VL comprising the amino acid sequence of SEQ ID NO: 765; a linker comprising the amino acid sequence of (Gly-Gly-Gly-Gly-Ser)n (SEQ ID NO: 1146), wherein n is 4; and a VH comprising the amino acid sequence of SEQ ID NO: 992. In some embodiments, the anti-CD3 antibody, or antigen-binding fragment thereof, comprises ABL-CD3-78A_scFv_VH-VL. In some embodiments, the anti-CD3 antibody, or antigen-binding fragment thereof, comprises, from N-terminus to C-terminus, a VH comprising the amino acid sequence of SEQ ID NO: 1105; a linker comprising the amino acid sequence of (Gly-Gly-Gly-Gly-Ser)n (SEQ ID NO: 1146), wherein n is 4; and a VL comprising the amino acid sequence of SEQ ID NO: 878. In some embodiments, the anti-CD3 antibody, or antigen-binding fragment thereof, comprises ABL-CD3-78A_scFv_VL-VH. In some embodiments, the anti-CD3 antibody, or antigen-binding fragment thereof, comprises, from N-terminus to C-terminus, a VL comprising the amino acid sequence of SEQ ID NO: 878; a linker comprising the amino acid sequence of (Gly-Gly-Gly-Gly-Ser)n (SEQ ID NO: 1146), wherein n is 4; and a VH comprising the amino acid sequence of SEQ ID NO: 1105.

In some embodiments, the anti-CD3 antibody, or antigen-binding fragment thereof, comprises the six CDRs of antibody ABL-CD3-79. The anti-CD3 antibody, or antigen-binding fragment thereof, may comprise an LCDR1, an LCDR2, and an LCDR3 comprising the amino acid sequences of SEQ ID NOs: 79, 192, and 305, respectively; and an HCDR1, an HCDR2, and an HCDR3 comprising the amino acid sequences of SEQ ID NOs: 423, 536, and 652, respectively. In some embodiments, the anti-CD3 antibody, or antigen-binding fragment thereof, comprises the six CDRs of antibody ABL-CD3-79A. The anti-CD3 antibody, or antigen-binding fragment thereof, may comprise an LCDR1, an LCDR2, and an LCDR3 comprising the amino acid sequences of SEQ ID NOs: 79, 192, and 305, respectively; and an HCDR1, an HCDR2, and an HCDR3 comprising the amino acid sequences of SEQ ID NOs: 423, 573, and 652, respectively. In some embodiments, the anti-CD3 antibody, or antigen-binding fragment thereof, comprises the VL of antibody ABL-CD3-79. The VL may comprise the amino acid sequence of SEQ ID NO: 766. In some embodiments, the anti-CD3 antibody, or antigen-binding fragment thereof, comprises the VH of antibody ABL-CD3-79. The VH may comprise the amino acid sequence of SEQ ID NO: 993. In some embodiments, the anti-CD3 antibody, or antigen-binding fragment thereof, comprises the VL and VH of antibody ABL-CD3-79. In some embodiments, the anti-CD3 antibody, or antigen-binding fragment thereof, comprises a VL comprising the amino acid sequence of SEQ ID NO: 766 and a VH comprising the amino acid sequence of SEQ ID NO: 993. In some embodiments, the anti-CD3 antibody, or antigen-binding fragment thereof, comprises the VL of antibody ABL-CD3-79A. The VL may comprise the amino acid sequence of SEQ ID NO: 879. In some embodiments, the anti-CD3 antibody, or antigen-binding fragment thereof, comprises the VH of antibody ABL-CD3-79A. The VH may comprise the amino acid sequence of SEQ ID NO: 1106. In some embodiments, the anti-CD3 antibody, or antigen-binding fragment thereof, comprises the VL and VH of antibody ABL-CD3-79A. In some embodiments, the anti-CD3 antibody, or antigen-binding fragment thereof, comprises a VL comprising the amino acid sequence of SEQ ID NO: 879 and a VH comprising the amino acid sequence of SEQ ID NO: 1106. In some embodiments, the anti-CD3 antibody, or antigen-binding fragment thereof, comprises ABL-CD3-79_scFv_VH-VL. In some embodiments, the anti-CD3 antibody, or antigen-binding fragment thereof, comprises, from N-terminus to C-terminus, a VH comprising the amino acid sequence of SEQ ID NO: 993; a linker comprising the amino acid sequence of (Gly-Gly-Gly-Gly-Ser)n (SEQ ID NO: 1146), wherein n is 4; and a VL comprising the amino acid sequence of SEQ ID NO: 766. In some embodiments, the anti-CD3 antibody, or antigen-binding fragment thereof, comprises ABL-CD3-79_scFv_VL-VH. In some embodiments, the anti-CD3 antibody, or antigen-binding fragment thereof, comprises, from N-terminus to C-terminus, a VL comprising the amino acid sequence of SEQ ID NO: 766; a linker comprising the amino acid sequence of (Gly-Gly-Gly-Gly-Ser)n (SEQ ID NO: 1146), wherein n is 4; and a VH comprising the amino acid sequence of SEQ ID NO: 993. In some embodiments, the anti-CD3 antibody, or antigen-binding fragment thereof, comprises ABL-CD3-79A_scFv_VH-VL. In some embodiments, the anti-CD3 antibody, or antigen-binding fragment thereof, comprises the amino acid sequence of SEQ ID NO: 1189. In some embodiments, the anti-CD3 antibody, or antigen-binding fragment thereof, comprises ABL-CD3-79A_scFv_VL-VH. In some embodiments, the anti-CD3 antibody, or antigen-binding fragment thereof, comprises the amino acid sequence of SEQ ID NO: 1230.

In some embodiments, the anti-CD3 antibody, or antigen-binding fragment thereof, comprises the six CDRs of antibody ABL-CD3-80 and ABL-CD3-80A. The anti-CD3 antibody, or antigen-binding fragment thereof, may comprise an LCDR1, an LCDR2, and an LCDR3 comprising the amino acid sequences of SEQ ID NOs: 80, 193, and 306, respectively; and an HCDR1, an HCDR2, and an HCDR3 comprising the amino acid sequences of SEQ ID NOs: 424, 537, and 653, respectively. In some embodiments, the anti-CD3 antibody, or antigen-binding fragment thereof, comprises the VL of antibody ABL-CD3-80. The VL may comprise the amino acid sequence of SEQ ID NO: 767. In some embodiments, the anti-CD3 antibody, or antigen-binding fragment thereof, comprises the VH of antibody ABL-CD3-80. The VH may comprise the amino acid sequence of SEQ ID NO: 994. In some embodiments, the anti-CD3 antibody, or antigen-binding fragment thereof, comprises the VL and VH of antibody ABL-CD3-80. In some embodiments, the anti-CD3 antibody, or antigen-binding fragment thereof, comprises a VL comprising the amino acid sequence of SEQ ID NO: 767 and a VH comprising the amino acid sequence of SEQ ID NO: 994. In some embodiments, the anti-CD3 antibody, or antigen-binding fragment thereof, comprises the VL of antibody ABL-CD3-80A. The VL may comprise the amino acid sequence of SEQ ID NO: 880. In some embodiments, the anti-CD3 antibody, or antigen-binding fragment thereof, comprises the VH of antibody ABL-CD3-80A. The VH may comprise the amino acid sequence of SEQ ID NO: 1107. In some embodiments, the anti-CD3 antibody, or antigen-binding fragment thereof, comprises the VL and VH of antibody ABL-CD3-80A. In some embodiments, the anti-CD3 antibody, or antigen-binding fragment thereof, comprises a VL comprising the amino acid sequence of SEQ ID NO: 880 and a VH comprising the amino acid sequence of SEQ ID NO: 1107. In some embodiments, the anti-CD3 antibody, or antigen-binding fragment thereof, comprises ABL-CD3-80_scFv_VH-VL. In some embodiments, the anti-CD3 antibody, or antigen-binding fragment thereof, comprises, from N-terminus to C-terminus, a VH comprising the amino acid sequence of SEQ ID NO: 994; a linker comprising the amino acid sequence of (Gly-Gly-Gly-Gly-Ser)n (SEQ ID NO: 1146), wherein n is 4; and a VL comprising the amino acid sequence of SEQ ID NO: 767. In some embodiments, the anti-CD3 antibody, or antigen-binding fragment thereof, comprises ABL-CD3-80_scFv_VL-VH. In some embodiments, the anti-CD3 antibody, or antigen-binding fragment thereof, comprises, from N-terminus to C-terminus, a VL comprising the amino acid sequence of SEQ ID NO: 767; a linker comprising the amino acid sequence of (Gly-Gly-Gly-Gly-Ser)n (SEQ ID NO: 1146), wherein n is 4; and a VH comprising the amino acid sequence of SEQ ID NO: 994. In some embodiments, the anti-CD3 antibody, or antigen-binding fragment thereof, comprises ABL-CD3-80A_scFv_VH-VL. In some embodiments, the anti-CD3 antibody, or antigen-binding fragment thereof, comprises, from N-terminus to C-terminus, a VH comprising the amino acid sequence of SEQ ID NO: 1107; a linker comprising the amino acid sequence of (Gly-Gly-Gly-Gly-Ser)n (SEQ ID NO: 1146), wherein n is 4; and a VL comprising the amino acid sequence of SEQ ID NO: 880. In some embodiments, the anti-CD3 antibody, or antigen-binding fragment thereof, comprises ABL-CD3-80A_scFv_VL-VH. In some embodiments, the anti-CD3 antibody, or antigen-binding fragment thereof, comprises, from N-terminus to C-terminus, a VL comprising the amino acid sequence of SEQ ID NO: 880; a linker comprising the amino acid sequence of (Gly-Gly-Gly-Gly-Ser)n (SEQ ID NO: 1146), wherein n is 4; and a VH comprising the amino acid sequence of SEQ ID NO: 1107.

In some embodiments, the anti-CD3 antibody, or antigen-binding fragment thereof, comprises the six CDRs of antibody ABL-CD3-81 and ABL-CD3-81A. The anti-CD3 antibody, or antigen-binding fragment thereof, may comprise an LCDR1, an LCDR2, and an LCDR3 comprising the amino acid sequences of SEQ ID NOs: 81, 194, and 307, respectively; and an HCDR1, an HCDR2, and an HCDR3 comprising the amino acid sequences of SEQ ID NOs: 425, 538, and 654, respectively. In some embodiments, the anti-CD3 antibody, or antigen-binding fragment thereof, comprises the VL of antibody ABL-CD3-81. The VL may comprise the amino acid sequence of SEQ ID NO: 768. In some embodiments, the anti-CD3 antibody, or antigen-binding fragment thereof, comprises the VH of antibody ABL-CD3-81. The VH may comprise the amino acid sequence of SEQ ID NO: 995. In some embodiments, the anti-CD3 antibody, or antigen-binding fragment thereof, comprises the VL and VH of antibody ABL-CD3-81. In some embodiments, the anti-CD3 antibody, or antigen-binding fragment thereof, comprises a VL comprising the amino acid sequence of SEQ ID NO: 768 and a VH comprising the amino acid sequence of SEQ ID NO: 995. In some embodiments, the anti-CD3 antibody, or antigen-binding fragment thereof, comprises the VL of antibody ABL-CD3-81A. The VL may comprise the amino acid sequence of SEQ ID NO: 881. In some embodiments, the anti-CD3 antibody, or antigen-binding fragment thereof, comprises the VH of antibody ABL-CD3-81A. The VH may comprise the amino acid sequence of SEQ ID NO: 1108. In some embodiments, the anti-CD3 antibody, or antigen-binding fragment thereof, comprises the VL and VH of antibody ABL-CD3-81A. In some embodiments, the anti-CD3 antibody, or antigen-binding fragment thereof, comprises a VL comprising the amino acid sequence of SEQ ID NO: 881 and a VH comprising the amino acid sequence of SEQ ID NO: 1108. In some embodiments, the anti-CD3 antibody, or antigen-binding fragment thereof, comprises ABL-CD3-81_scFv_VH-VL. In some embodiments, the anti-CD3 antibody, or antigen-binding fragment thereof, comprises, from N-terminus to C-terminus, a VH comprising the amino acid sequence of SEQ ID NO: 995; a linker comprising the amino acid sequence of (Gly-Gly-Gly-Gly-Ser)n (SEQ ID NO: 1146), wherein n is 4; and a VL comprising the amino acid sequence of SEQ ID NO: 768. In some embodiments, the anti-CD3 antibody, or antigen-binding fragment thereof, comprises ABL-CD3-81_scFv_VL-VH. In some embodiments, the anti-CD3 antibody, or antigen-binding fragment thereof, comprises, from N-terminus to C-terminus, a VL comprising the amino acid sequence of SEQ ID NO: 768; a linker comprising the amino acid sequence of (Gly-Gly-Gly-Gly-Ser)n (SEQ ID NO: 1146), wherein n is 4; and a VH comprising the amino acid sequence of SEQ ID NO: 995. In some embodiments, the anti-CD3 antibody, or antigen-binding fragment thereof, comprises ABL-CD3-81A_scFv_VH-VL. In some embodiments, the anti-CD3 antibody, or antigen-binding fragment thereof, comprises the amino acid sequence of SEQ ID NO: 1190. In some embodiments, the anti-CD3 antibody, or antigen-binding fragment thereof, comprises ABL-CD3-81A_scFv_VL-VH. In some embodiments, the anti-CD3 antibody, or antigen-binding fragment thereof, comprises the amino acid sequence of SEQ ID NO: 1231.

In some embodiments, the anti-CD3 antibody, or antigen-binding fragment thereof, comprises the six CDRs of antibody ABL-CD3-82 and ABL-CD3-82A. The anti-CD3 antibody, or antigen-binding fragment thereof, may comprise an LCDR1, an LCDR2, and an LCDR3 comprising the amino acid sequences of SEQ ID NOs: 82, 195, and 308, respectively; and an HCDR1, an HCDR2, and an HCDR3 comprising the amino acid sequences of SEQ ID NOs: 426, 539, and 655, respectively. In some embodiments, the anti-CD3 antibody, or antigen-binding fragment thereof, comprises the VL of antibody ABL-CD3-82. The VL may comprise the amino acid sequence of SEQ ID NO: 769. In some embodiments, the anti-CD3 antibody, or antigen-binding fragment thereof, comprises the VH of antibody ABL-CD3-82. The VH may comprise the amino acid sequence of SEQ ID NO: 996. In some embodiments, the anti-CD3 antibody, or antigen-binding fragment thereof, comprises the VL and VH of antibody ABL-CD3-82. In some embodiments, the anti-CD3 antibody, or antigen-binding fragment thereof, comprises a VL comprising the amino acid sequence of SEQ ID NO: 769 and a VH comprising the amino acid sequence of SEQ ID NO: 996. In some embodiments, the anti-CD3 antibody, or antigen-binding fragment thereof, comprises the VL of antibody ABL-CD3-82A. The VL may comprise the amino acid sequence of SEQ ID NO: 882. In some embodiments, the anti-CD3 antibody, or antigen-binding fragment thereof, comprises the VH of antibody ABL-CD3-82A. The VH may comprise the amino acid sequence of SEQ ID NO: 1109. In some embodiments, the anti-CD3 antibody, or antigen-binding fragment thereof, comprises the VL and VH of antibody ABL-CD3-82A. In some embodiments, the anti-CD3 antibody, or antigen-binding fragment thereof, comprises a VL comprising the amino acid sequence of SEQ ID NO: 882 and a VH comprising the amino acid sequence of SEQ ID NO: 1109. In some embodiments, the anti-CD3 antibody, or antigen-binding fragment thereof, comprises ABL-CD3-82_scFv_VH-VL. In some embodiments, the anti-CD3 antibody, or antigen-binding fragment thereof, comprises, from N-terminus to C-terminus, a VH comprising the amino acid sequence of SEQ ID NO: 996; a linker comprising the amino acid sequence of (Gly-Gly-Gly-Gly-Ser)n (SEQ ID NO: 1146), wherein n is 4; and a VL comprising the amino acid sequence of SEQ ID NO: 769. In some embodiments, the anti-CD3 antibody, or antigen-binding fragment thereof, comprises ABL-CD3-82_scFv_VL-VH. In some embodiments, the anti-CD3 antibody, or antigen-binding fragment thereof, comprises, from N-terminus to C-terminus, a VL comprising the amino acid sequence of SEQ ID NO: 769; a linker comprising the amino acid sequence of (Gly-Gly-Gly-Gly-Ser)n (SEQ ID NO: 1146), wherein n is 4; and a VH comprising the amino acid sequence of SEQ ID NO: 996. In some embodiments, the anti-CD3 antibody, or antigen-binding fragment thereof, comprises ABL-CD3-82A_scFv_VH-VL. In some embodiments, the anti-CD3 antibody, or antigen-binding fragment thereof, comprises, from N-terminus to C-terminus, a VH comprising the amino acid sequence of SEQ ID NO: 1109; a linker comprising the amino acid sequence of (Gly-Gly-Gly-Gly-Ser)n (SEQ ID NO: 1146), wherein n is 4; and a VL comprising the amino acid sequence of SEQ ID NO: 882. In some embodiments, the anti-CD3 antibody, or antigen-binding fragment thereof, comprises ABL-CD3-82A_scFv_VL-VH. In some embodiments, the anti-CD3 antibody, or antigen-binding fragment thereof, comprises, from N-terminus to C-terminus, a VL comprising the amino acid sequence of SEQ ID NO: 882; a linker comprising the amino acid sequence of (Gly-Gly-Gly-Gly-Ser)n (SEQ ID NO: 1146), wherein n is 4; and a VH comprising the amino acid sequence of SEQ ID NO: 1109.

In some embodiments, the anti-CD3 antibody, or antigen-binding fragment thereof, comprises the six CDRs of antibody ABL-CD3-83 and ABL-CD3-83A. The anti-CD3 antibody, or antigen-binding fragment thereof, may comprise an LCDR1, an LCDR2, and an LCDR3 comprising the amino acid sequences of SEQ ID NOs: 83, 196, and 309, respectively; and an HCDR1, an HCDR2, and an HCDR3 comprising the amino acid sequences of SEQ ID NOs: 427, 540, and 656, respectively. In some embodiments, the anti-CD3 antibody, or antigen-binding fragment thereof, comprises the VL of antibody ABL-CD3-83. The VL may comprise the amino acid sequence of SEQ ID NO: 770. In some embodiments, the anti-CD3 antibody, or antigen-binding fragment thereof, comprises the VH of antibody ABL-CD3-83. The VH may comprise the amino acid sequence of SEQ ID NO: 997. In some embodiments, the anti-CD3 antibody, or antigen-binding fragment thereof, comprises the VL and VH of antibody ABL-CD3-83. In some embodiments, the anti-CD3 antibody, or antigen-binding fragment thereof, comprises a VL comprising the amino acid sequence of SEQ ID NO: 770 and a VH comprising the amino acid sequence of SEQ ID NO: 997. In some embodiments, the anti-CD3 antibody, or antigen-binding fragment thereof, comprises the VL of antibody ABL-CD3-83A. The VL may comprise the amino acid sequence of SEQ ID NO: 883. In some embodiments, the anti-CD3 antibody, or antigen-binding fragment thereof, comprises the VH of antibody ABL-CD3-83A. The VH may comprise the amino acid sequence of SEQ ID NO: 1110. In some embodiments, the anti-CD3 antibody, or antigen-binding fragment thereof, comprises the VL and VH of antibody ABL-CD3-83A. In some embodiments, the anti-CD3 antibody, or antigen-binding fragment thereof, comprises a VL comprising the amino acid sequence of SEQ ID NO: 883 and a VH comprising the amino acid sequence of SEQ ID NO: 1110. In some embodiments, the anti-CD3 antibody, or antigen-binding fragment thereof, comprises ABL-CD3-83_scFv_VH-VL. In some embodiments, the anti-CD3 antibody, or antigen-binding fragment thereof, comprises, from N-terminus to C-terminus, a VH comprising the amino acid sequence of SEQ ID NO: 997; a linker comprising the amino acid sequence of (Gly-Gly-Gly-Gly-Ser)n (SEQ ID NO: 1146), wherein n is 4; and a VL comprising the amino acid sequence of SEQ ID NO: 770. In some embodiments, the anti-CD3 antibody, or antigen-binding fragment thereof, comprises ABL-CD3-83_scFv_VL-VH. In some embodiments, the anti-CD3 antibody, or antigen-binding fragment thereof, comprises, from N-terminus to C-terminus, a VL comprising the amino acid sequence of SEQ ID NO: 770; a linker comprising the amino acid sequence of (Gly-Gly-Gly-Gly-Ser)n (SEQ ID NO: 1146), wherein n is 4; and a VH comprising the amino acid sequence of SEQ ID NO: 997. In some embodiments, the anti-CD3 antibody, or antigen-binding fragment thereof, comprises ABL-CD3-83A_scFv_VH-VL. In some embodiments, the anti-CD3 antibody, or antigen-binding fragment thereof, comprises, from N-terminus to C-terminus, a VH comprising the amino acid sequence of SEQ ID NO: 1110; a linker comprising the amino acid sequence of (Gly-Gly-Gly-Gly-Ser)n (SEQ ID NO: 1146), wherein n is 4; and a VL comprising the amino acid sequence of SEQ ID NO: 883. In some embodiments, the anti-CD3 antibody, or antigen-binding fragment thereof, comprises ABL-CD3-83A_scFv_VL-VH. In some embodiments, the anti-CD3 antibody, or antigen-binding fragment thereof, comprises, from N-terminus to C-terminus, a VL comprising the amino acid sequence of SEQ ID NO: 883; a linker comprising the amino acid sequence of (Gly-Gly-Gly-Gly-Ser)n (SEQ ID NO: 1146), wherein n is 4; and a VH comprising the amino acid sequence of SEQ ID NO: 1110.

In some embodiments, the anti-CD3 antibody, or antigen-binding fragment thereof, comprises the six CDRs of antibody ABL-CD3-84 and ABL-CD3-84A. The anti-CD3 antibody, or antigen-binding fragment thereof, may comprise an LCDR1, an LCDR2, and an LCDR3 comprising the amino acid sequences of SEQ ID NOs: 84, 197, and 310, respectively; and an HCDR1, an HCDR2, and an HCDR3 comprising the amino acid sequences of SEQ ID NOs: 428, 541, and 657, respectively. In some embodiments, the anti-CD3 antibody, or antigen-binding fragment thereof, comprises the VL of antibody ABL-CD3-84. The VL may comprise the amino acid sequence of SEQ ID NO: 771. In some embodiments, the anti-CD3 antibody, or antigen-binding fragment thereof, comprises the VH of antibody ABL-CD3-84. The VH may comprise the amino acid sequence of SEQ ID NO: 998. In some embodiments, the anti-CD3 antibody, or antigen-binding fragment thereof, comprises the VL and VH of antibody ABL-CD3-84. In some embodiments, the anti-CD3 antibody, or antigen-binding fragment thereof, comprises a VL comprising the amino acid sequence of SEQ ID NO: 771 and a VH comprising the amino acid sequence of SEQ ID NO: 998. In some embodiments, the anti-CD3 antibody, or antigen-binding fragment thereof, comprises the VL of antibody ABL-CD3-84A. The VL may comprise the amino acid sequence of SEQ ID NO: 884. In some embodiments, the anti-CD3 antibody, or antigen-binding fragment thereof, comprises the VH of antibody ABL-CD3-84A. The VH may comprise the amino acid sequence of SEQ ID NO: 1111. In some embodiments, the anti-CD3 antibody, or antigen-binding fragment thereof, comprises the VL and VH of antibody ABL-CD3-84A. In some embodiments, the anti-CD3 antibody, or antigen-binding fragment thereof, comprises a VL comprising the amino acid sequence of SEQ ID NO: 884 and a VH comprising the amino acid sequence of SEQ ID NO: 1111. In some embodiments, the anti-CD3 antibody, or antigen-binding fragment thereof, comprises ABL-CD3-84_scFv_VH-VL. In some embodiments, the anti-CD3 antibody, or antigen-binding fragment thereof, comprises, from N-terminus to C-terminus, a VH comprising the amino acid sequence of SEQ ID NO: 998; a linker comprising the amino acid sequence of (Gly-Gly-Gly-Gly-Ser)n (SEQ ID NO: 1146), wherein n is 4; and a VL comprising the amino acid sequence of SEQ ID NO: 771. In some embodiments, the anti-CD3 antibody, or antigen-binding fragment thereof, comprises ABL-CD3-84_scFv_VL-VH. In some embodiments, the anti-CD3 antibody, or antigen-binding fragment thereof, comprises, from N-terminus to C-terminus, a VL comprising the amino acid sequence of SEQ ID NO: 771; a linker comprising the amino acid sequence of (Gly-Gly-Gly-Gly-Ser)n (SEQ ID NO: 1146), wherein n is 4; and a VH comprising the amino acid sequence of SEQ ID NO: 998. In some embodiments, the anti-CD3 antibody, or antigen-binding fragment thereof, comprises ABL-CD3-84A_scFv_VH-VL. In some embodiments, the anti-CD3 antibody, or antigen-binding fragment thereof, comprises, from N-terminus to C-terminus, a VH comprising the amino acid sequence of SEQ ID NO: 1111; a linker comprising the amino acid sequence of (Gly-Gly-Gly-Gly-Ser)n (SEQ ID NO: 1146), wherein n is 4; and a VL comprising the amino acid sequence of SEQ ID NO: 884. In some embodiments, the anti-CD3 antibody, or antigen-binding fragment thereof, comprises ABL-CD3-84A_scFv_VL-VH. In some embodiments, the anti-CD3 antibody, or antigen-binding fragment thereof, comprises, from N-terminus to C-terminus, a VL comprising the amino acid sequence of SEQ ID NO: 884; a linker comprising the amino acid sequence of (Gly-Gly-Gly-Gly-Ser)n (SEQ ID NO: 1146), wherein n is 4; and a VH comprising the amino acid sequence of SEQ ID NO: 1111.

In some embodiments, the anti-CD3 antibody, or antigen-binding fragment thereof, comprises the six CDRs of antibody ABL-CD3-85. The anti-CD3 antibody, or antigen-binding fragment thereof, may comprise an LCDR1, an LCDR2, and an LCDR3 comprising the amino acid sequences of SEQ ID NOs: 85, 198, and 311, respectively; and an HCDR1, an HCDR2, and an HCDR3 comprising the amino acid sequences of SEQ ID NOs: 429, 542, and 658, respectively. In some embodiments, the anti-CD3 antibody, or antigen-binding fragment thereof, comprises the six CDRs of antibody ABL-CD3-85A. The anti-CD3 antibody, or antigen-binding fragment thereof, may comprise an LCDR1, an LCDR2, and an LCDR3 comprising the amino acid sequences of SEQ ID NOs: 85, 198, and 343, respectively; and an HCDR1, an HCDR2, and an HCDR3 comprising the amino acid sequences of SEQ ID NOs: 429, 542, and 658, respectively. In some embodiments, the anti-CD3 antibody, or antigen-binding fragment thereof, comprises the six CDRs of antibody ABL-CD3-85B. The anti-CD3 antibody, or antigen-binding fragment thereof, may comprise an LCDR1, an LCDR2, and an LCDR3 comprising the amino acid sequences of SEQ ID NOs: 85, 198, and 344, respectively; and an HCDR1, an HCDR2, and an HCDR3 comprising the amino acid sequences of SEQ ID NOs: 429, 542, and 658, respectively. In some embodiments, the anti-CD3 antibody, or antigen-binding fragment thereof, comprises the VL of antibody ABL-CD3-85. The VL may comprise the amino acid sequence of SEQ ID NO: 772. In some embodiments, the anti-CD3 antibody, or antigen-binding fragment thereof, comprises the VH of antibody ABL-CD3-85. The VH may comprise the amino acid sequence of SEQ ID NO: 999. In some embodiments, the anti-CD3 antibody, or antigen-binding fragment thereof, comprises the VL and VH of antibody ABL-CD3-85. In some embodiments, the anti-CD3 antibody, or antigen-binding fragment thereof, comprises a VL comprising the amino acid sequence of SEQ ID NO: 772 and a VH comprising the amino acid sequence of SEQ ID NO: 999. In some embodiments, the anti-CD3 antibody, or antigen-binding fragment thereof, comprises the VL of antibody ABL-CD3-85A. The VL may comprise the amino acid sequence of SEQ ID NO: 885. In some embodiments, the anti-CD3 antibody, or antigen-binding fragment thereof, comprises the VH of antibody ABL-CD3-85A. The VH may comprise the amino acid sequence of SEQ ID NO: 1112. In some embodiments, the anti-CD3 antibody, or antigen-binding fragment thereof, comprises the VL and VH of antibody ABL-CD3-85A. In some embodiments, the anti-CD3 antibody, or antigen-binding fragment thereof, comprises a VL comprising the amino acid sequence of SEQ ID NO: 885 and a VH comprising the amino acid sequence of SEQ ID NO: 1112. In some embodiments, the anti-CD3 antibody, or antigen-binding fragment thereof, comprises the VL of antibody ABL-CD3-85B. The VL may comprise the amino acid sequence of SEQ ID NO: 886. In some embodiments, the anti-CD3 antibody, or antigen-binding fragment thereof, comprises the VH of antibody ABL-CD3-85B. The VH may comprise the amino acid sequence of SEQ ID NO: 1113. In some embodiments, the anti-CD3 antibody, or antigen-binding fragment thereof, comprises the VL and VH of antibody ABL-CD3-85B. In some embodiments, the anti-CD3 antibody, or antigen-binding fragment thereof, comprises a VL comprising the amino acid sequence of SEQ ID NO: 886 and a VH comprising the amino acid sequence of SEQ ID NO: 1113. In some embodiments, the anti-CD3 antibody, or antigen-binding fragment thereof, comprises ABL-CD3-85_scFv_VH-VL. In some embodiments, the anti-CD3 antibody, or antigen-binding fragment thereof, comprises, from N-terminus to C-terminus, a VH comprising the amino acid sequence of SEQ ID NO: 999; a linker comprising the amino acid sequence of (Gly-Gly-Gly-Gly-Ser)n (SEQ ID NO: 1146), wherein n is 4; and a VL comprising the amino acid sequence of SEQ ID NO: 772. In some embodiments, the anti-CD3 antibody, or antigen-binding fragment thereof, comprises ABL-CD3-85_scFv_VL-VH. In some embodiments, the anti-CD3 antibody, or antigen-binding fragment thereof, comprises, from N-terminus to C-terminus, a VL comprising the amino acid sequence of SEQ ID NO: 772; a linker comprising the amino acid sequence of (Gly-Gly-Gly-Gly-Ser)n (SEQ ID NO: 1146), wherein n is 4; and a VH comprising the amino acid sequence of SEQ ID NO: 999. In some embodiments, the anti-CD3 antibody, or antigen-binding fragment thereof, comprises ABL-CD3-85A_scFv_VH-VL. In some embodiments, the anti-CD3 antibody, or antigen-binding fragment thereof, comprises the amino acid sequence of SEQ ID NO: 1191. In some embodiments, the anti-CD3 antibody, or antigen-binding fragment thereof, comprises ABL-CD3-85A_scFv_VL-VH. In some embodiments, the anti-CD3 antibody, or antigen-binding fragment thereof, comprises the amino acid sequence of SEQ ID NO: 1232. In some embodiments, the anti-CD3 antibody, or antigen-binding fragment thereof, comprises ABL-CD3-85B_scFv_VH-VL. In some embodiments, the anti-CD3 antibody, or antigen-binding fragment thereof, comprises the amino acid sequence of SEQ ID NO: 1192. In some embodiments, the anti-CD3 antibody, or antigen-binding fragment thereof, comprises ABL-CD3-85B_scFv_VL-VH. In some embodiments, the anti-CD3 antibody, or antigen-binding fragment thereof, comprises the amino acid sequence of SEQ ID NO: 1233.

In some embodiments, the anti-CD3 antibody, or antigen-binding fragment thereof, comprises the six CDRs of antibody ABL-CD3-86 and ABL-CD3-86A. The anti-CD3 antibody, or antigen-binding fragment thereof, may comprise an LCDR1, an LCDR2, and an LCDR3 comprising the amino acid sequences of SEQ ID NOs: 86, 199, and 312, respectively; and an HCDR1, an HCDR2, and an HCDR3 comprising the amino acid sequences of SEQ ID NOs: 430, 543, and 659, respectively. In some embodiments, the anti-CD3 antibody, or antigen-binding fragment thereof, comprises the VL of antibody ABL-CD3-86. The VL may comprise the amino acid sequence of SEQ ID NO: 773. In some embodiments, the anti-CD3 antibody, or antigen-binding fragment thereof, comprises the VH of antibody ABL-CD3-86. The VH may comprise the amino acid sequence of SEQ ID NO: 1000. In some embodiments, the anti-CD3 antibody, or antigen-binding fragment thereof, comprises the VL and VH of antibody ABL-CD3-86. In some embodiments, the anti-CD3 antibody, or antigen-binding fragment thereof, comprises a VL comprising the amino acid sequence of SEQ ID NO: 773 and a VH comprising the amino acid sequence of SEQ ID NO: 1000. In some embodiments, the anti-CD3 antibody, or antigen-binding fragment thereof, comprises the VL of antibody ABL-CD3-86A. The VL may comprise the amino acid sequence of SEQ ID NO: 887. In some embodiments, the anti-CD3 antibody, or antigen-binding fragment thereof, comprises the VH of antibody ABL-CD3-86A. The VH may comprise the amino acid sequence of SEQ ID NO: 1114. In some embodiments, the anti-CD3 antibody, or antigen-binding fragment thereof, comprises the VL and VH of antibody ABL-CD3-86A. In some embodiments, the anti-CD3 antibody, or antigen-binding fragment thereof, comprises a VL comprising the amino acid sequence of SEQ ID NO: 887 and a VH comprising the amino acid sequence of SEQ ID NO: 1114. In some embodiments, the anti-CD3 antibody, or antigen-binding fragment thereof, comprises ABL-CD3-86_scFv_VH-VL. In some embodiments, the anti-CD3 antibody, or antigen-binding fragment thereof, comprises, from N-terminus to C-terminus, a VH comprising the amino acid sequence of SEQ ID NO: 1000; a linker comprising the amino acid sequence of (Gly-Gly-Gly-Gly-Ser)n (SEQ ID NO: 1146), wherein n is 4; and a VL comprising the amino acid sequence of SEQ ID NO: 773. In some embodiments, the anti-CD3 antibody, or antigen-binding fragment thereof, comprises ABL-CD3-86_scFv_VL-VH. In some embodiments, the anti-CD3 antibody, or antigen-binding fragment thereof, comprises, from N-terminus to C-terminus, a VL comprising the amino acid sequence of SEQ ID NO: 773; a linker comprising the amino acid sequence of (Gly-Gly-Gly-Gly-Ser)n (SEQ ID NO: 1146), wherein n is 4; and a VH comprising the amino acid sequence of SEQ ID NO: 1000. In some embodiments, the anti-CD3 antibody, or antigen-binding fragment thereof, comprises ABL-CD3-86A_scFv_VH-VL. In some embodiments, the anti-CD3 antibody, or antigen-binding fragment thereof, comprises, from N-terminus to C-terminus, a VH comprising the amino acid sequence of SEQ ID NO: 1114; a linker comprising the amino acid sequence of (Gly-Gly-Gly-Gly-Ser)n (SEQ ID NO: 1146), wherein n is 4; and a VL comprising the amino acid sequence of SEQ ID NO: 887. In some embodiments, the anti-CD3 antibody, or antigen-binding fragment thereof, comprises ABL-CD3-87A_scFv_VL-VH. In some embodiments, the anti-CD3 antibody, or antigen-binding fragment thereof, comprises, from N-terminus to C-terminus, a VL comprising the amino acid sequence of SEQ ID NO: 887; a linker comprising the amino acid sequence of (Gly-Gly-Gly-Gly-Ser)n (SEQ ID NO: 1146), wherein n is 4; and a VH comprising the amino acid sequence of SEQ ID NO: 1114.

In some embodiments, the anti-CD3 antibody, or antigen-binding fragment thereof, comprises the six CDRs of antibody ABL-CD3-87 and ABL-CD3-87A. The anti-CD3 antibody, or antigen-binding fragment thereof, may comprise an LCDR1, an LCDR2, and an LCDR3 comprising the amino acid sequences of SEQ ID NOs: 87, 200, and 313, respectively; and an HCDR1, an HCDR2, and an HCDR3 comprising the amino acid sequences of SEQ ID NOs: 431, 544, and 660, respectively. In some embodiments, the anti-CD3 antibody, or antigen-binding fragment thereof, comprises the VL of antibody ABL-CD3-87. The VL may comprise the amino acid sequence of SEQ ID NO: 774. In some embodiments, the anti-CD3 antibody, or antigen-binding fragment thereof, comprises the VH of antibody ABL-CD3-87. The VH may comprise the amino acid sequence of SEQ ID NO: 1001. In some embodiments, the anti-CD3 antibody, or antigen-binding fragment thereof, comprises the VL and VH of antibody ABL-CD3-87. In some embodiments, the anti-CD3 antibody, or antigen-binding fragment thereof, comprises a VL comprising the amino acid sequence of SEQ ID NO: 774 and a VH comprising the amino acid sequence of SEQ ID NO: 1001. In some embodiments, the anti-CD3 antibody, or antigen-binding fragment thereof, comprises the VL of antibody ABL-CD3-87A. The VL may comprise the amino acid sequence of SEQ ID NO: 888. In some embodiments, the anti-CD3 antibody, or antigen-binding fragment thereof, comprises the VH of antibody ABL-CD3-87A. The VH may comprise the amino acid sequence of SEQ ID NO: 1115. In some embodiments, the anti-CD3 antibody, or antigen-binding fragment thereof, comprises the VL and VH of antibody ABL-CD3-87A. In some embodiments, the anti-CD3 antibody, or antigen-binding fragment thereof, comprises a VL comprising the amino acid sequence of SEQ ID NO: 888 and a VH comprising the amino acid sequence of SEQ ID NO: 1115. In some embodiments, the anti-CD3 antibody, or antigen-binding fragment thereof, comprises ABL-CD3-87_scFv_VH-VL. In some embodiments, the anti-CD3 antibody, or antigen-binding fragment thereof, comprises, from N-terminus to C-terminus, a VH comprising the amino acid sequence of SEQ ID NO: 1001; a linker comprising the amino acid sequence of (Gly-Gly-Gly-Gly-Ser)n (SEQ ID NO: 1146), wherein n is 4; and a VL comprising the amino acid sequence of SEQ ID NO: 774. In some embodiments, the anti-CD3 antibody, or antigen-binding fragment thereof, comprises ABL-CD3-87_scFv_VL-VH. In some embodiments, the anti-CD3 antibody, or antigen-binding fragment thereof, comprises, from N-terminus to C-terminus, a VL comprising the amino acid sequence of SEQ ID NO: 774; a linker comprising the amino acid sequence of (Gly-Gly-Gly-Gly-Ser)n (SEQ ID NO: 1146), wherein n is 4; and a VH comprising the amino acid sequence of SEQ ID NO: 1001. In some embodiments, the anti-CD3 antibody, or antigen-binding fragment thereof, comprises ABL-CD3-87A_scFv_VH-VL. In some embodiments, the anti-CD3 antibody, or antigen-binding fragment thereof, comprises the amino acid sequence of SEQ ID NO: 1193. In some embodiments, the anti-CD3 antibody, or antigen-binding fragment thereof, comprises ABL-CD3-87A_scFv_VL-VH. In some embodiments, the anti-CD3 antibody, or antigen-binding fragment thereof, comprises the amino acid sequence of SEQ ID NO: 1234.

In some embodiments, the anti-CD3 antibody, or antigen-binding fragment thereof, comprises the six CDRs of antibody ABL-CD3-88 and ABL-CD3-88A. The anti-CD3 antibody, or antigen-binding fragment thereof, may comprise an LCDR1, an LCDR2, and an LCDR3 comprising the amino acid sequences of SEQ ID NOs: 88, 201, and 314, respectively; and an HCDR1, an HCDR2, and an HCDR3 comprising the amino acid sequences of SEQ ID NOs: 432, 545, and 661, respectively. In some embodiments, the anti-CD3 antibody, or antigen-binding fragment thereof, comprises the VL of antibody ABL-CD3-88. The VL may comprise the amino acid sequence of SEQ ID NO: 775. In some embodiments, the anti-CD3 antibody, or antigen-binding fragment thereof, comprises the VH of antibody ABL-CD3-88. The VH may comprise the amino acid sequence of SEQ ID NO: 1002. In some embodiments, the anti-CD3 antibody, or antigen-binding fragment thereof, comprises the VL and VH of antibody ABL-CD3-88. In some embodiments, the anti-CD3 antibody, or antigen-binding fragment thereof, comprises a VL comprising the amino acid sequence of SEQ ID NO: 775 and a VH comprising the amino acid sequence of SEQ ID NO: 1002. In some embodiments, the anti-CD3 antibody, or antigen-binding fragment thereof, comprises the VL of antibody ABL-CD3-88A. The VL may comprise the amino acid sequence of SEQ ID NO: 889. In some embodiments, the anti-CD3 antibody, or antigen-binding fragment thereof, comprises the VH of antibody ABL-CD3-88A. The VH may comprise the amino acid sequence of SEQ ID NO: 1116. In some embodiments, the anti-CD3 antibody, or antigen-binding fragment thereof, comprises the VL and VH of antibody ABL-CD3-88A. In some embodiments, the anti-CD3 antibody, or antigen-binding fragment thereof, comprises a VL comprising the amino acid sequence of SEQ ID NO: 889 and a VH comprising the amino acid sequence of SEQ ID NO: 1116. In some embodiments, the anti-CD3 antibody, or antigen-binding fragment thereof, comprises ABL-CD3-88_scFv_VH-VL. In some embodiments, the anti-CD3 antibody, or antigen-binding fragment thereof, comprises, from N-terminus to C-terminus, a VH comprising the amino acid sequence of SEQ ID NO: 1002; a linker comprising the amino acid sequence of (Gly-Gly-Gly-Gly-Ser)n (SEQ ID NO: 1146), wherein n is 4; and a VL comprising the amino acid sequence of SEQ ID NO: 775. In some embodiments, the anti-CD3 antibody, or antigen-binding fragment thereof, comprises ABL-CD3-88_scFv_VL-VH. In some embodiments, the anti-CD3 antibody, or antigen-binding fragment thereof, comprises, from N-terminus to C-terminus, a VL comprising the amino acid sequence of SEQ ID NO: 775; a linker comprising the amino acid sequence of (Gly-Gly-Gly-Gly-Ser)n (SEQ ID NO: 1146), wherein n is 4; and a VH comprising the amino acid sequence of SEQ ID NO: 1002. In some embodiments, the anti-CD3 antibody, or antigen-binding fragment thereof, comprises ABL-CD3-88A_scFv_VH-VL. In some embodiments, the anti-CD3 antibody, or antigen-binding fragment thereof, comprises the amino acid sequence of SEQ ID NO: 1194. In some embodiments, the anti-CD3 antibody, or antigen-binding fragment thereof, comprises ABL-CD3-88A_scFv_VL-VH. In some embodiments, the anti-CD3 antibody, or antigen-binding fragment thereof, comprises the amino acid sequence of SEQ ID NO: 1235.

In some embodiments, the anti-CD3 antibody, or antigen-binding fragment thereof, comprises the six CDRs of antibody ABL-CD3-89 and ABL-CD3-89A. The anti-CD3 antibody, or antigen-binding fragment thereof, may comprise an LCDR1, an LCDR2, and an LCDR3 comprising the amino acid sequences of SEQ ID NOs: 89, 202, and 315, respectively; and an HCDR1, an HCDR2, and an HCDR3 comprising the amino acid sequences of SEQ ID NOs: 433, 546, and 662, respectively. In some embodiments, the anti-CD3 antibody, or antigen-binding fragment thereof, comprises the VL of antibody ABL-CD3-89. The VL may comprise the amino acid sequence of SEQ ID NO: 776. In some embodiments, the anti-CD3 antibody, or antigen-binding fragment thereof, comprises the VH of antibody ABL-CD3-89. The VH may comprise the amino acid sequence of SEQ ID NO: 1003. In some embodiments, the anti-CD3 antibody, or antigen-binding fragment thereof, comprises the VL and VH of antibody ABL-CD3-89. In some embodiments, the anti-CD3 antibody, or antigen-binding fragment thereof, comprises a VL comprising the amino acid sequence of SEQ ID NO: 776 and a VH comprising the amino acid sequence of SEQ ID NO: 1003. In some embodiments, the anti-CD3 antibody, or antigen-binding fragment thereof, comprises the VL of antibody ABL-CD3-89A. The VL may comprise the amino acid sequence of SEQ ID NO: 890. In some embodiments, the anti-CD3 antibody, or antigen-binding fragment thereof, comprises the VH of antibody ABL-CD3-89A. The VH may comprise the amino acid sequence of SEQ ID NO: 1117. In some embodiments, the anti-CD3 antibody, or antigen-binding fragment thereof, comprises the VL and VH of antibody ABL-CD3-89A. In some embodiments, the anti-CD3 antibody, or antigen-binding fragment thereof, comprises a VL comprising the amino acid sequence of SEQ ID NO: 890 and a VH comprising the amino acid sequence of SEQ ID NO: 1117. In some embodiments, the anti-CD3 antibody, or antigen-binding fragment thereof, comprises ABL-CD3-89_scFv_VH-VL. In some embodiments, the anti-CD3 antibody, or antigen-binding fragment thereof, comprises, from N-terminus to C-terminus, a VH comprising the amino acid sequence of SEQ ID NO: 1003; a linker comprising the amino acid sequence of (Gly-Gly-Gly-Gly-Ser)n (SEQ ID NO: 1146), wherein n is 4; and a VL comprising the amino acid sequence of SEQ ID NO: 776. In some embodiments, the anti-CD3 antibody, or antigen-binding fragment thereof, comprises ABL-CD3-89_scFv_VL-VH. In some embodiments, the anti-CD3 antibody, or antigen-binding fragment thereof, comprises, from N-terminus to C-terminus, a VL comprising the amino acid sequence of SEQ ID NO: 776; a linker comprising the amino acid sequence of (Gly-Gly-Gly-Gly-Ser)n (SEQ ID NO: 1146), wherein n is 4; and a VH comprising the amino acid sequence of SEQ ID NO: 1003. In some embodiments, the anti-CD3 antibody, or antigen-binding fragment thereof, comprises ABL-CD3-89A_scFv_VH-VL. In some embodiments, the anti-CD3 antibody, or antigen-binding fragment thereof, comprises the amino acid sequence of SEQ ID NO: 1195. In some embodiments, the anti-CD3 antibody, or antigen-binding fragment thereof, comprises ABL-CD3-89A_scFv_VL-VH. In some embodiments, the anti-CD3 antibody, or antigen-binding fragment thereof, comprises the amino acid sequence of SEQ ID NO: 1236.

In some embodiments, the anti-CD3 antibody, or antigen-binding fragment thereof, comprises the six CDRs of antibody ABL-CD3-90 and ABL-CD3-90A. The anti-CD3 antibody, or antigen-binding fragment thereof, may comprise an LCDR1, an LCDR2, and an LCDR3 comprising the amino acid sequences of SEQ ID NOs: 90, 203, and 316, respectively; and an HCDR1, an HCDR2, and an HCDR3 comprising the amino acid sequences of SEQ ID NOs: 434, 547, and 663, respectively. In some embodiments, the anti-CD3 antibody, or antigen-binding fragment thereof, comprises the VL of antibody ABL-CD3-90. The VL may comprise the amino acid sequence of SEQ ID NO: 777. In some embodiments, the anti-CD3 antibody, or antigen-binding fragment thereof, comprises the VH of antibody ABL-CD3-90. The VH may comprise the amino acid sequence of SEQ ID NO: 1004. In some embodiments, the anti-CD3 antibody, or antigen-binding fragment thereof, comprises the VL and VH of antibody ABL-CD3-90. In some embodiments, the anti-CD3 antibody, or antigen-binding fragment thereof, comprises a VL comprising the amino acid sequence of SEQ ID NO: 777 and a VH comprising the amino acid sequence of SEQ ID NO: 1004. In some embodiments, the anti-CD3 antibody, or antigen-binding fragment thereof, comprises the VL of antibody ABL-CD3-90A. The VL may comprise the amino acid sequence of SEQ ID NO: 891. In some embodiments, the anti-CD3 antibody, or antigen-binding fragment thereof, comprises the VH of antibody ABL-CD3-90A. The VH may comprise the amino acid sequence of SEQ ID NO: 1118. In some embodiments, the anti-CD3 antibody, or antigen-binding fragment thereof, comprises the VL and VH of antibody ABL-CD3-90A. In some embodiments, the anti-CD3 antibody, or antigen-binding fragment thereof, comprises a VL comprising the amino acid sequence of SEQ ID NO: 891 and a VH comprising the amino acid sequence of SEQ ID NO: 1118. In some embodiments, the anti-CD3 antibody, or antigen-binding fragment thereof, comprises ABL-CD3-90_scFv_VH-VL. In some embodiments, the anti-CD3 antibody, or antigen-binding fragment thereof, comprises, from N-terminus to C-terminus, a VH comprising the amino acid sequence of SEQ ID NO: 1004; a linker comprising the amino acid sequence of (Gly-Gly-Gly-Gly-Ser)n (SEQ ID NO: 1146), wherein n is 4; and a VL comprising the amino acid sequence of SEQ ID NO: 777. In some embodiments, the anti-CD3 antibody, or antigen-binding fragment thereof, comprises ABL-CD3-90_scFv_VL-VH. In some embodiments, the anti-CD3 antibody, or antigen-binding fragment thereof, comprises, from N-terminus to C-terminus, a VL comprising the amino acid sequence of SEQ ID NO: 777; a linker comprising the amino acid sequence of (Gly-Gly-Gly-Gly-Ser)n (SEQ ID NO: 1146), wherein n is 4; and a VH comprising the amino acid sequence of SEQ ID NO: 1004. In some embodiments, the anti-CD3 antibody, or antigen-binding fragment thereof, comprises ABL-CD3-90A_scFv_VH-VL. In some embodiments, the anti-CD3 antibody, or antigen-binding fragment thereof, comprises, from N-terminus to C-terminus, a VH comprising the amino acid sequence of SEQ ID NO: 1118; a linker comprising the amino acid sequence of (Gly-Gly-Gly-Gly-Ser)n (SEQ ID NO: 1146), wherein n is 4; and a VL comprising the amino acid sequence of SEQ ID NO: 891. In some embodiments, the anti-CD3 antibody, or antigen-binding fragment thereof, comprises ABL-CD3-90A_scFv_VL-VH. In some embodiments, the anti-CD3 antibody, or antigen-binding fragment thereof, comprises, from N-terminus to C-terminus, a VL comprising the amino acid sequence of SEQ ID NO: 891; a linker comprising the amino acid sequence of (Gly-Gly-Gly-Gly-Ser)n (SEQ ID NO: 1146), wherein n is 4; and a VH comprising the amino acid sequence of SEQ ID NO: 1118.

In some embodiments, the anti-CD3 antibody, or antigen-binding fragment thereof, comprises the six CDRs of antibody ABL-CD3-91 and ABL-CD3-91A. The anti-CD3 antibody, or antigen-binding fragment thereof, may comprise an LCDR1, an LCDR2, and an LCDR3 comprising the amino acid sequences of SEQ ID NOs: 91, 204, and 317, respectively; and an HCDR1, an HCDR2, and an HCDR3 comprising the amino acid sequences of SEQ ID NOs: 435, 548, and 664, respectively. In some embodiments, the anti-CD3 antibody, or antigen-binding fragment thereof, comprises the VL of antibody ABL-CD3-91. The VL may comprise the amino acid sequence of SEQ ID NO: 778. In some embodiments, the anti-CD3 antibody, or antigen-binding fragment thereof, comprises the VH of antibody ABL-CD3-91. The VH may comprise the amino acid sequence of SEQ ID NO: 1005. In some embodiments, the anti-CD3 antibody, or antigen-binding fragment thereof, comprises the VL and VH of antibody ABL-CD3-91. In some embodiments, the anti-CD3 antibody, or antigen-binding fragment thereof, comprises a VL comprising the amino acid sequence of SEQ ID NO: 778 and a VH comprising the amino acid sequence of SEQ ID NO: 1005. In some embodiments, the anti-CD3 antibody, or antigen-binding fragment thereof, comprises the VL of antibody ABL-CD3-91A. The VL may comprise the amino acid sequence of SEQ ID NO: 892. In some embodiments, the anti-CD3 antibody, or antigen-binding fragment thereof, comprises the VH of antibody ABL-CD3-91A. The VH may comprise the amino acid sequence of SEQ ID NO: 1119. In some embodiments, the anti-CD3 antibody, or antigen-binding fragment thereof, comprises the VL and VH of antibody ABL-CD3-91A. In some embodiments, the anti-CD3 antibody, or antigen-binding fragment thereof, comprises a VL comprising the amino acid sequence of SEQ ID NO: 892 and a VH comprising the amino acid sequence of SEQ ID NO: 1119. In some embodiments, the anti-CD3 antibody, or antigen-binding fragment thereof, comprises ABL-CD3-91_scFv_VH-VL. In some embodiments, the anti-CD3 antibody, or antigen-binding fragment thereof, comprises, from N-terminus to C-terminus, a VH comprising the amino acid sequence of SEQ ID NO: 1005; a linker comprising the amino acid sequence of (Gly-Gly-Gly-Gly-Ser)n (SEQ ID NO: 1146), wherein n is 4; and a VL comprising the amino acid sequence of SEQ ID NO: 778. In some embodiments, the anti-CD3 antibody, or antigen-binding fragment thereof, comprises ABL-CD3-91_scFv_VL-VH. In some embodiments, the anti-CD3 antibody, or antigen-binding fragment thereof, comprises, from N-terminus to C-terminus, a VL comprising the amino acid sequence of SEQ ID NO: 778; a linker comprising the amino acid sequence of (Gly-Gly-Gly-Gly-Ser)n (SEQ ID NO: 1146), wherein n is 4; and a VH comprising the amino acid sequence of SEQ ID NO: 1005. In some embodiments, the anti-CD3 antibody, or antigen-binding fragment thereof, comprises ABL-CD3-91A_scFv_VH-VL. In some embodiments, the anti-CD3 antibody, or antigen-binding fragment thereof, comprises, from N-terminus to C-terminus, a VH comprising the amino acid sequence of SEQ ID NO: 1119; a linker comprising the amino acid sequence of (Gly-Gly-Gly-Gly-Ser)n (SEQ ID NO: 1146), wherein n is 4; and a VL comprising the amino acid sequence of SEQ ID NO: 892. In some embodiments, the anti-CD3 antibody, or antigen-binding fragment thereof, comprises ABL-CD3-91A_scFv_VL-VH. In some embodiments, the anti-CD3 antibody, or antigen-binding fragment thereof, comprises, from N-terminus to C-terminus, a VL comprising the amino acid sequence of SEQ ID NO: 892; a linker comprising the amino acid sequence of (Gly-Gly-Gly-Gly-Ser)n (SEQ ID NO: 1146), wherein n is 4; and a VH comprising the amino acid sequence of SEQ ID NO: 1119.

In some embodiments, the anti-CD3 antibody, or antigen-binding fragment thereof, comprises the six CDRs of antibody ABL-CD3-92 and ABL-CD3-92A. The anti-CD3 antibody, or antigen-binding fragment thereof, may comprise an LCDR1, an LCDR2, and an LCDR3 comprising the amino acid sequences of SEQ ID NOs: 92, 205, and 318, respectively; and an HCDR1, an HCDR2, and an HCDR3 comprising the amino acid sequences of SEQ ID NOs: 436, 549, and 665, respectively. In some embodiments, the anti-CD3 antibody, or antigen-binding fragment thereof, comprises the VL of antibody ABL-CD3-92. The VL may comprise the amino acid sequence of SEQ ID NO: 779. In some embodiments, the anti-CD3 antibody, or antigen-binding fragment thereof, comprises the VH of antibody ABL-CD3-92. The VH may comprise the amino acid sequence of SEQ ID NO: 1006. In some embodiments, the anti-CD3 antibody, or antigen-binding fragment thereof, comprises the VL and VH of antibody ABL-CD3-92. In some embodiments, the anti-CD3 antibody, or antigen-binding fragment thereof, comprises a VL comprising the amino acid sequence of SEQ ID NO: 779 and a VH comprising the amino acid sequence of SEQ ID NO: 1006. In some embodiments, the anti-CD3 antibody, or antigen-binding fragment thereof, comprises the VL of antibody ABL-CD3-92A. The VL may comprise the amino acid sequence of SEQ ID NO: 893. In some embodiments, the anti-CD3 antibody, or antigen-binding fragment thereof, comprises the VH of antibody ABL-CD3-92A. The VH may comprise the amino acid sequence of SEQ ID NO: 1120. In some embodiments, the anti-CD3 antibody, or antigen-binding fragment thereof, comprises the VL and VH of antibody ABL-CD3-92A. In some embodiments, the anti-CD3 antibody, or antigen-binding fragment thereof, comprises a VL comprising the amino acid sequence of SEQ ID NO: 893 and a VH comprising the amino acid sequence of SEQ ID NO: 1120. In some embodiments, the anti-CD3 antibody, or antigen-binding fragment thereof, comprises ABL-CD3-92_scFv_VH-VL. In some embodiments, the anti-CD3 antibody, or antigen-binding fragment thereof, comprises, from N-terminus to C-terminus, a VH comprising the amino acid sequence of SEQ ID NO: 1006; a linker comprising the amino acid sequence of (Gly-Gly-Gly-Gly-Ser)n (SEQ ID NO: 1146), wherein n is 4; and a VL comprising the amino acid sequence of SEQ ID NO: 779. In some embodiments, the anti-CD3 antibody, or antigen-binding fragment thereof, comprises ABL-CD3-92_scFv_VL-VH. In some embodiments, the anti-CD3 antibody, or antigen-binding fragment thereof, comprises, from N-terminus to C-terminus, a VL comprising the amino acid sequence of SEQ ID NO: 779; a linker comprising the amino acid sequence of (Gly-Gly-Gly-Gly-Ser)n (SEQ ID NO: 1146), wherein n is 4; and a VH comprising the amino acid sequence of SEQ ID NO: 1006. In some embodiments, the anti-CD3 antibody, or antigen-binding fragment thereof, comprises ABL-CD3-92A_scFv_VH-VL. In some embodiments, the anti-CD3 antibody, or antigen-binding fragment thereof, comprises, from N-terminus to C-terminus, a VH comprising the amino acid sequence of SEQ ID NO: 1120; a linker comprising the amino acid sequence of (Gly-Gly-Gly-Gly-Ser)n (SEQ ID NO: 1146), wherein n is 4; and a VL comprising the amino acid sequence of SEQ ID NO: 893. In some embodiments, the anti-CD3 antibody, or antigen-binding fragment thereof, comprises ABL-CD3-92A_scFv_VL-VH. In some embodiments, the anti-CD3 antibody, or antigen-binding fragment thereof, comprises, from N-terminus to C-terminus, a VL comprising the amino acid sequence of SEQ ID NO: 893; a linker comprising the amino acid sequence of (Gly-Gly-Gly-Gly-Ser)n (SEQ ID NO: 1146), wherein n is 4; and a VH comprising the amino acid sequence of SEQ ID NO: 1120.

In some embodiments, the anti-CD3 antibody, or antigen-binding fragment thereof, comprises the six CDRs of antibody ABL-CD3-93 and ABL-CD3-93A. The anti-CD3 antibody, or antigen-binding fragment thereof, may comprise an LCDR1, an LCDR2, and an LCDR3 comprising the amino acid sequences of SEQ ID NOs: 93, 206, and 319, respectively; and an HCDR1, an HCDR2, and an HCDR3 comprising the amino acid sequences of SEQ ID NOs: 437, 550, and 666, respectively. In some embodiments, the anti-CD3 antibody, or antigen-binding fragment thereof, comprises the VL of antibody ABL-CD3-93. The VL may comprise the amino acid sequence of SEQ ID NO: 780. In some embodiments, the anti-CD3 antibody, or antigen-binding fragment thereof, comprises the VH of antibody ABL-CD3-93. The VH may comprise the amino acid sequence of SEQ ID NO: 1007. In some embodiments, the anti-CD3 antibody, or antigen-binding fragment thereof, comprises the VL and VH of antibody ABL-CD3-93. In some embodiments, the anti-CD3 antibody, or antigen-binding fragment thereof, comprises a VL comprising the amino acid sequence of SEQ ID NO: 780 and a VH comprising the amino acid sequence of SEQ ID NO: 1007. In some embodiments, the anti-CD3 antibody, or antigen-binding fragment thereof, comprises the VL of antibody ABL-CD3-93A. The VL may comprise the amino acid sequence of SEQ ID NO: 894. In some embodiments, the anti-CD3 antibody, or antigen-binding fragment thereof, comprises the VH of antibody ABL-CD3-93A. The VH may comprise the amino acid sequence of SEQ ID NO: 1121. In some embodiments, the anti-CD3 antibody, or antigen-binding fragment thereof, comprises the VL and VH of antibody ABL-CD3-93A. In some embodiments, the anti-CD3 antibody, or antigen-binding fragment thereof, comprises a VL comprising the amino acid sequence of SEQ ID NO: 894 and a VH comprising the amino acid sequence of SEQ ID NO: 1121. In some embodiments, the anti-CD3 antibody, or antigen-binding fragment thereof, comprises ABL-CD3-93_scFv_VH-VL. In some embodiments, the anti-CD3 antibody, or antigen-binding fragment thereof, comprises, from N-terminus to C-terminus, a VH comprising the amino acid sequence of SEQ ID NO: 1007; a linker comprising the amino acid sequence of (Gly-Gly-Gly-Gly-Ser)n (SEQ ID NO: 1146), wherein n is 4; and a VL comprising the amino acid sequence of SEQ ID NO: 780. In some embodiments, the anti-CD3 antibody, or antigen-binding fragment thereof, comprises ABL-CD3-93_scFv_VL-VH. In some embodiments, the anti-CD3 antibody, or antigen-binding fragment thereof, comprises, from N-terminus to C-terminus, a VL comprising the amino acid sequence of SEQ ID NO: 780; a linker comprising the amino acid sequence of (Gly-Gly-Gly-Gly-Ser)n (SEQ ID NO: 1146), wherein n is 4; and a VH comprising the amino acid sequence of SEQ ID NO: 1007. In some embodiments, the anti-CD3 antibody, or antigen-binding fragment thereof, comprises ABL-CD3-93A_scFv_VH-VL. In some embodiments, the anti-CD3 antibody, or antigen-binding fragment thereof, comprises, from N-terminus to C-terminus, a VH comprising the amino acid sequence of SEQ ID NO: 1121; a linker comprising the amino acid sequence of (Gly-Gly-Gly-Gly-Ser)n (SEQ ID NO: 1146), wherein n is 4; and a VL comprising the amino acid sequence of SEQ ID NO: 894. In some embodiments, the anti-CD3 antibody, or antigen-binding fragment thereof, comprises ABL-CD3-93A_scFv_VL-VH. In some embodiments, the anti-CD3 antibody, or antigen-binding fragment thereof, comprises, from N-terminus to C-terminus, a VL comprising the amino acid sequence of SEQ ID NO: 894; a linker comprising the amino acid sequence of (Gly-Gly-Gly-Gly-Ser)n (SEQ ID NO: 1146), wherein n is 4; and a VH comprising the amino acid sequence of SEQ ID NO: 1121.

In some embodiments, the anti-CD3 antibody, or antigen-binding fragment thereof, comprises the six CDRs of antibody ABL-CD3-94 and ABL-CD3-94A. The anti-CD3 antibody, or antigen-binding fragment thereof, may comprise an LCDR1, an LCDR2, and an LCDR3 comprising the amino acid sequences of SEQ ID NOs: 94, 207, and 320, respectively; and an HCDR1, an HCDR2, and an HCDR3 comprising the amino acid sequences of SEQ ID NOs: 438, 551, and 667, respectively. In some embodiments, the anti-CD3 antibody, or antigen-binding fragment thereof, comprises the VL of antibody ABL-CD3-94. The VL may comprise the amino acid sequence of SEQ ID NO: 781. In some embodiments, the anti-CD3 antibody, or antigen-binding fragment thereof, comprises the VH of antibody ABL-CD3-94. The VH may comprise the amino acid sequence of SEQ ID NO: 1008. In some embodiments, the anti-CD3 antibody, or antigen-binding fragment thereof, comprises the VL and VH of antibody ABL-CD3-94. In some embodiments, the anti-CD3 antibody, or antigen-binding fragment thereof, comprises a VL comprising the amino acid sequence of SEQ ID NO: 781 and a VH comprising the amino acid sequence of SEQ ID NO: 1008. In some embodiments, the anti-CD3 antibody, or antigen-binding fragment thereof, comprises the VL of antibody ABL-CD3-94A. The VL may comprise the amino acid sequence of SEQ ID NO: 895. In some embodiments, the anti-CD3 antibody, or antigen-binding fragment thereof, comprises the VH of antibody ABL-CD3-94A. The VH may comprise the amino acid sequence of SEQ ID NO: 1122. In some embodiments, the anti-CD3 antibody, or antigen-binding fragment thereof, comprises the VL and VH of antibody ABL-CD3-94A. In some embodiments, the anti-CD3 antibody, or antigen-binding fragment thereof, comprises a VL comprising the amino acid sequence of SEQ ID NO: 895 and a VH comprising the amino acid sequence of SEQ ID NO: 1122. In some embodiments, the anti-CD3 antibody, or antigen-binding fragment thereof, comprises ABL-CD3-94_scFv_VH-VL. In some embodiments, the anti-CD3 antibody, or antigen-binding fragment thereof, comprises, from N-terminus to C-terminus, a VH comprising the amino acid sequence of SEQ ID NO: 1008; a linker comprising the amino acid sequence of (Gly-Gly-Gly-Gly-Ser)n (SEQ ID NO: 1146), wherein n is 4; and a VL comprising the amino acid sequence of SEQ ID NO: 781. In some embodiments, the anti-CD3 antibody, or antigen-binding fragment thereof, comprises ABL-CD3-94_scFv_VL-VH. In some embodiments, the anti-CD3 antibody, or antigen-binding fragment thereof, comprises, from N-terminus to C-terminus, a VL comprising the amino acid sequence of SEQ ID NO: 781; a linker comprising the amino acid sequence of (Gly-Gly-Gly-Gly-Ser)n (SEQ ID NO: 1146), wherein n is 4; and a VH comprising the amino acid sequence of SEQ ID NO: 1008. In some embodiments, the anti-CD3 antibody, or antigen-binding fragment thereof, comprises ABL-CD3-94A_scFv_VH-VL. In some embodiments, the anti-CD3 antibody, or antigen-binding fragment thereof, comprises, from N-terminus to C-terminus, a VH comprising the amino acid sequence of SEQ ID NO: 1122; a linker comprising the amino acid sequence of (Gly-Gly-Gly-Gly-Ser)n (SEQ ID NO: 1146), wherein n is 4; and a VL comprising the amino acid sequence of SEQ ID NO: 895. In some embodiments, the anti-CD3 antibody, or antigen-binding fragment thereof, comprises ABL-CD3-94A_scFv_VL-VH. In some embodiments, the anti-CD3 antibody, or antigen-binding fragment thereof, comprises, from N-terminus to C-terminus, a VL comprising the amino acid sequence of SEQ ID NO: 895; a linker comprising the amino acid sequence of (Gly-Gly-Gly-Gly-Ser)n (SEQ ID NO: 1146), wherein n is 4; and a VH comprising the amino acid sequence of SEQ ID NO: 1122.

In some embodiments, the anti-CD3 antibody, or antigen-binding fragment thereof, comprises the six CDRs of antibody ABL-CD3-95 and ABL-CD3-95A. The anti-CD3 antibody, or antigen-binding fragment thereof, may comprise an LCDR1, an LCDR2, and an LCDR3 comprising the amino acid sequences of SEQ ID NOs: 95, 208, and 321, respectively; and an HCDR1, an HCDR2, and an HCDR3 comprising the amino acid sequences of SEQ ID NOs: 439, 552, and 668, respectively. In some embodiments, the anti-CD3 antibody, or antigen-binding fragment thereof, comprises the VL of antibody ABL-CD3-95. The VL may comprise the amino acid sequence of SEQ ID NO: 782. In some embodiments, the anti-CD3 antibody, or antigen-binding fragment thereof, comprises the VH of antibody ABL-CD3-95. The VH may comprise the amino acid sequence of SEQ ID NO: 1009. In some embodiments, the anti-CD3 antibody, or antigen-binding fragment thereof, comprises the VL and VH of antibody ABL-CD3-95. In some embodiments, the anti-CD3 antibody, or antigen-binding fragment thereof, comprises a VL comprising the amino acid sequence of SEQ ID NO: 782 and a VH comprising the amino acid sequence of SEQ ID NO: 1009. In some embodiments, the anti-CD3 antibody, or antigen-binding fragment thereof, comprises the VL of antibody ABL-CD3-95A. The VL may comprise the amino acid sequence of SEQ ID NO: 896. In some embodiments, the anti-CD3 antibody, or antigen-binding fragment thereof, comprises the VH of antibody ABL-CD3-95A. The VH may comprise the amino acid sequence of SEQ ID NO: 1123. In some embodiments, the anti-CD3 antibody, or antigen-binding fragment thereof, comprises the VL and VH of antibody ABL-CD3-95A. In some embodiments, the anti-CD3 antibody, or antigen-binding fragment thereof, comprises a VL comprising the amino acid sequence of SEQ ID NO: 896 and a VH comprising the amino acid sequence of SEQ ID NO: 1123. In some embodiments, the anti-CD3 antibody, or antigen-binding fragment thereof, comprises ABL-CD3-95_scFv_VH-VL. In some embodiments, the anti-CD3 antibody, or antigen-binding fragment thereof, comprises, from N-terminus to C-terminus, a VH comprising the amino acid sequence of SEQ ID NO: 1009; a linker comprising the amino acid sequence of (Gly-Gly-Gly-Gly-Ser)n (SEQ ID NO: 1146), wherein n is 4; and a VL comprising the amino acid sequence of SEQ ID NO: 782. In some embodiments, the anti-CD3 antibody, or antigen-binding fragment thereof, comprises ABL-CD3-95_scFv_VL-VH. In some embodiments, the anti-CD3 antibody, or antigen-binding fragment thereof, comprises, from N-terminus to C-terminus, a VL comprising the amino acid sequence of SEQ ID NO: 782; a linker comprising the amino acid sequence of (Gly-Gly-Gly-Gly-Ser)n (SEQ ID NO: 1146), wherein n is 4; and a VH comprising the amino acid sequence of SEQ ID NO: 1009. In some embodiments, the anti-CD3 antibody, or antigen-binding fragment thereof, comprises ABL-CD3-95A_scFv_VH-VL. In some embodiments, the anti-CD3 antibody, or antigen-binding fragment thereof, comprises, from N-terminus to C-terminus, a VH comprising the amino acid sequence of SEQ ID NO: 1123; a linker comprising the amino acid sequence of (Gly-Gly-Gly-Gly-Ser)n (SEQ ID NO: 1146), wherein n is 4; and a VL comprising the amino acid sequence of SEQ ID NO: 896. In some embodiments, the anti-CD3 antibody, or antigen-binding fragment thereof, comprises ABL-CD3-95A_scFv_VL-VH. In some embodiments, the anti-CD3 antibody, or antigen-binding fragment thereof, comprises, from N-terminus to C-terminus, a VL comprising the amino acid sequence of SEQ ID NO: 896; a linker comprising the amino acid sequence of (Gly-Gly-Gly-Gly-Ser)n (SEQ ID NO: 1146), wherein n is 4; and a VH comprising the amino acid sequence of SEQ ID NO: 1123.

In some embodiments, the anti-CD3 antibody, or antigen-binding fragment thereof, comprises the six CDRs of antibody ABL-CD3-96 and ABL-CD3-96A. The anti-CD3 antibody, or antigen-binding fragment thereof, may comprise an LCDR1, an LCDR2, and an LCDR3 comprising the amino acid sequences of SEQ ID NOs: 96, 209, and 322, respectively; and an HCDR1, an HCDR2, and an HCDR3 comprising the amino acid sequences of SEQ ID NOs: 440, 553, and 669, respectively. In some embodiments, the anti-CD3 antibody, or antigen-binding fragment thereof, comprises the VL of antibody ABL-CD3-96. The VL may comprise the amino acid sequence of SEQ ID NO: 783. In some embodiments, the anti-CD3 antibody, or antigen-binding fragment thereof, comprises the VH of antibody ABL-CD3-96. The VH may comprise the amino acid sequence of SEQ ID NO: 1010. In some embodiments, the anti-CD3 antibody, or antigen-binding fragment thereof, comprises the VL and VH of antibody ABL-CD3-96. In some embodiments, the anti-CD3 antibody, or antigen-binding fragment thereof, comprises a VL comprising the amino acid sequence of SEQ ID NO: 783 and a VH comprising the amino acid sequence of SEQ ID NO: 1010. In some embodiments, the anti-CD3 antibody, or antigen-binding fragment thereof, comprises the VL of antibody ABL-CD3-96A. The VL may comprise the amino acid sequence of SEQ ID NO: 897. In some embodiments, the anti-CD3 antibody, or antigen-binding fragment thereof, comprises the VH of antibody ABL-CD3-96A. The VH may comprise the amino acid sequence of SEQ ID NO: 1124. In some embodiments, the anti-CD3 antibody, or antigen-binding fragment thereof, comprises the VL and VH of antibody ABL-CD3-96A. In some embodiments, the anti-CD3 antibody, or antigen-binding fragment thereof, comprises a VL comprising the amino acid sequence of SEQ ID NO: 897 and a VH comprising the amino acid sequence of SEQ ID NO: 1124. In some embodiments, the anti-CD3 antibody, or antigen-binding fragment thereof, comprises ABL-CD3-96_scFv_VH-VL. In some embodiments, the anti-CD3 antibody, or antigen-binding fragment thereof, comprises, from N-terminus to C-terminus, a VH comprising the amino acid sequence of SEQ ID NO: 1010; a linker comprising the amino acid sequence of (Gly-Gly-Gly-Gly-Ser)n (SEQ ID NO: 1146), wherein n is 4; and a VL comprising the amino acid sequence of SEQ ID NO: 783. In some embodiments, the anti-CD3 antibody, or antigen-binding fragment thereof, comprises ABL-CD3-96_scFv_VL-VH. In some embodiments, the anti-CD3 antibody, or antigen-binding fragment thereof, comprises, from N-terminus to C-terminus, a VL comprising the amino acid sequence of SEQ ID NO: 783; a linker comprising the amino acid sequence of (Gly-Gly-Gly-Gly-Ser)n (SEQ ID NO: 1146), wherein n is 4; and a VH comprising the amino acid sequence of SEQ ID NO: 1010. In some embodiments, the anti-CD3 antibody, or antigen-binding fragment thereof, comprises ABL-CD3-96A_scFv_VH-VL. In some embodiments, the anti-CD3 antibody, or antigen-binding fragment thereof, comprises, from N-terminus to C-terminus, a VH comprising the amino acid sequence of SEQ ID NO: 1124; a linker comprising the amino acid sequence of (Gly-Gly-Gly-Gly-Ser)n (SEQ ID NO: 1146), wherein n is 4; and a VL comprising the amino acid sequence of SEQ ID NO: 897. In some embodiments, the anti-CD3 antibody, or antigen-binding fragment thereof, comprises ABL-CD3-96A_scFv_VL-VH. In some embodiments, the anti-CD3 antibody, or antigen-binding fragment thereof, comprises, from N-terminus to C-terminus, a VL comprising the amino acid sequence of SEQ ID NO: 897; a linker comprising the amino acid sequence of (Gly-Gly-Gly-Gly-Ser)n (SEQ ID NO: 1146), wherein n is 4; and a VH comprising the amino acid sequence of SEQ ID NO: 1124.

In some embodiments, the anti-CD3 antibody, or antigen-binding fragment thereof, comprises the six CDRs of antibody ABL-CD3-97 and ABL-CD3-97A. The anti-CD3 antibody, or antigen-binding fragment thereof, may comprise an LCDR1, an LCDR2, and an LCDR3 comprising the amino acid sequences of SEQ ID NOs: 97, 210, and 323, respectively; and an HCDR1, an HCDR2, and an HCDR3 comprising the amino acid sequences of SEQ ID NOs: 441, 554, and 670, respectively. In some embodiments, the anti-CD3 antibody, or antigen-binding fragment thereof, comprises the VL of antibody ABL-CD3-97. The VL may comprise the amino acid sequence of SEQ ID NO: 784. In some embodiments, the anti-CD3 antibody, or antigen-binding fragment thereof, comprises the VH of antibody ABL-CD3-97. The VH may comprise the amino acid sequence of SEQ ID NO: 1011. In some embodiments, the anti-CD3 antibody, or antigen-binding fragment thereof, comprises the VL and VH of antibody ABL-CD3-97. In some embodiments, the anti-CD3 antibody, or antigen-binding fragment thereof, comprises a VL comprising the amino acid sequence of SEQ ID NO: 784 and a VH comprising the amino acid sequence of SEQ ID NO: 1011. In some embodiments, the anti-CD3 antibody, or antigen-binding fragment thereof, comprises the VL of antibody ABL-CD3-97A. The VL may comprise the amino acid sequence of SEQ ID NO: 898. In some embodiments, the anti-CD3 antibody, or antigen-binding fragment thereof, comprises the VH of antibody ABL-CD3-97A. The VH may comprise the amino acid sequence of SEQ ID NO: 1125. In some embodiments, the anti-CD3 antibody, or antigen-binding fragment thereof, comprises the VL and VH of antibody ABL-CD3-97A. In some embodiments, the anti-CD3 antibody, or antigen-binding fragment thereof, comprises a VL comprising the amino acid sequence of SEQ ID NO: 898 and a VH comprising the amino acid sequence of SEQ ID NO: 1125. In some embodiments, the anti-CD3 antibody, or antigen-binding fragment thereof, comprises ABL-CD3-97_scFv_VH-VL. In some embodiments, the anti-CD3 antibody, or antigen-binding fragment thereof, comprises, from N-terminus to C-terminus, a VH comprising the amino acid sequence of SEQ ID NO: 1011; a linker comprising the amino acid sequence of (Gly-Gly-Gly-Gly-Ser)n (SEQ ID NO: 1146), wherein n is 4; and a VL comprising the amino acid sequence of SEQ ID NO: 784. In some embodiments, the anti-CD3 antibody, or antigen-binding fragment thereof, comprises ABL-CD3-97_scFv_VL-VH. In some embodiments, the anti-CD3 antibody, or antigen-binding fragment thereof, comprises, from N-terminus to C-terminus, a VL comprising the amino acid sequence of SEQ ID NO: 784; a linker comprising the amino acid sequence of (Gly-Gly-Gly-Gly-Ser)n (SEQ ID NO: 1146), wherein n is 4; and a VH comprising the amino acid sequence of SEQ ID NO: 1011. In some embodiments, the anti-CD3 antibody, or antigen-binding fragment thereof, comprises ABL-CD3-97A_scFv_VH-VL. In some embodiments, the anti-CD3 antibody, or antigen-binding fragment thereof, comprises, from N-terminus to C-terminus, a VH comprising the amino acid sequence of SEQ ID NO: 1125; a linker comprising the amino acid sequence of (Gly-Gly-Gly-Gly-Ser)n (SEQ ID NO: 1146), wherein n is 4; and a VL comprising the amino acid sequence of SEQ ID NO: 898. In some embodiments, the anti-CD3 antibody, or antigen-binding fragment thereof, comprises ABL-CD3-97A_scFv_VL-VH. In some embodiments, the anti-CD3 antibody, or antigen-binding fragment thereof, comprises, from N-terminus to C-terminus, a VL comprising the amino acid sequence of SEQ ID NO: 898; a linker comprising the amino acid sequence of (Gly-Gly-Gly-Gly-Ser)n (SEQ ID NO: 1146), wherein n is 4; and a VH comprising the amino acid sequence of SEQ ID NO: 1125.

In some embodiments, the anti-CD3 antibody, or antigen-binding fragment thereof, comprises the six CDRs of antibody ABL-CD3-98 and ABL-CD3-98A. The anti-CD3 antibody, or antigen-binding fragment thereof, may comprise an LCDR1, an LCDR2, and an LCDR3 comprising the amino acid sequences of SEQ ID NOs: 98, 211, and 324, respectively; and an HCDR1, an HCDR2, and an HCDR3 comprising the amino acid sequences of SEQ ID NOs: 442, 555, and 671, respectively. In some embodiments, the anti-CD3 antibody, or antigen-binding fragment thereof, comprises the VL of antibody ABL-CD3-98. The VL may comprise the amino acid sequence of SEQ ID NO: 785. In some embodiments, the anti-CD3 antibody, or antigen-binding fragment thereof, comprises the VH of antibody ABL-CD3-98. The VH may comprise the amino acid sequence of SEQ ID NO: 1012. In some embodiments, the anti-CD3 antibody, or antigen-binding fragment thereof, comprises the VL and VH of antibody ABL-CD3-98. In some embodiments, the anti-CD3 antibody, or antigen-binding fragment thereof, comprises a VL comprising the amino acid sequence of SEQ ID NO: 785 and a VH comprising the amino acid sequence of SEQ ID NO: 1012. In some embodiments, the anti-CD3 antibody, or antigen-binding fragment thereof, comprises the VL of antibody ABL-CD3-98A. The VL may comprise the amino acid sequence of SEQ ID NO: 899. In some embodiments, the anti-CD3 antibody, or antigen-binding fragment thereof, comprises the VH of antibody ABL-CD3-98A. The VH may comprise the amino acid sequence of SEQ ID NO: 1126. In some embodiments, the anti-CD3 antibody, or antigen-binding fragment thereof, comprises the VL and VH of antibody ABL-CD3-98A. In some embodiments, the anti-CD3 antibody, or antigen-binding fragment thereof, comprises a VL comprising the amino acid sequence of SEQ ID NO: 899 and a VH comprising the amino acid sequence of SEQ ID NO: 1126. In some embodiments, the anti-CD3 antibody, or antigen-binding fragment thereof, comprises ABL-CD3-98_scFv_VH-VL. In some embodiments, the anti-CD3 antibody, or antigen-binding fragment thereof, comprises, from N-terminus to C-terminus, a VH comprising the amino acid sequence of SEQ ID NO: 1012; a linker comprising the amino acid sequence of (Gly-Gly-Gly-Gly-Ser)n (SEQ ID NO: 1146), wherein n is 4; and a VL comprising the amino acid sequence of SEQ ID NO: 785. In some embodiments, the anti-CD3 antibody, or antigen-binding fragment thereof, comprises ABL-CD3-98_scFv_VL-VH. In some embodiments, the anti-CD3 antibody, or antigen-binding fragment thereof, comprises, from N-terminus to C-terminus, a VL comprising the amino acid sequence of SEQ ID NO: 785; a linker comprising the amino acid sequence of (Gly-Gly-Gly-Gly-Ser)n (SEQ ID NO: 1146), wherein n is 4; and a VH comprising the amino acid sequence of SEQ ID NO: 1012. In some embodiments, the anti-CD3 antibody, or antigen-binding fragment thereof, comprises ABL-CD3-98A_scFv_VH-VL. In some embodiments, the anti-CD3 antibody, or antigen-binding fragment thereof, comprises, from N-terminus to C-terminus, a VH comprising the amino acid sequence of SEQ ID NO: 1126; a linker comprising the amino acid sequence of (Gly-Gly-Gly-Gly-Ser)n (SEQ ID NO: 1146), wherein n is 4; and a VL comprising the amino acid sequence of SEQ ID NO: 899. In some embodiments, the anti-CD3 antibody, or antigen-binding fragment thereof, comprises ABL-CD3-98A_scFv_VL-VH. In some embodiments, the anti-CD3 antibody, or antigen-binding fragment thereof, comprises, from N-terminus to C-terminus, a VL comprising the amino acid sequence of SEQ ID NO: 899; a linker comprising the amino acid sequence of (Gly-Gly-Gly-Gly-Ser)n (SEQ ID NO: 1146), wherein n is 4; and a VH comprising the amino acid sequence of SEQ ID NO: 1126.

In some embodiments, the anti-CD3 antibody, or antigen-binding fragment thereof, comprises the six CDRs of antibody ABL-CD3-99 and ABL-CD3-99A. The anti-CD3 antibody, or antigen-binding fragment thereof, may comprise an LCDR1, an LCDR2, and an LCDR3 comprising the amino acid sequences of SEQ ID NOs: 99, 212, and 325, respectively; and an HCDR1, an HCDR2, and an HCDR3 comprising the amino acid sequences of SEQ ID NOs: 443, 556, and 672, respectively. In some embodiments, the anti-CD3 antibody, or antigen-binding fragment thereof, comprises the VL of antibody ABL-CD3-99. The VL may comprise the amino acid sequence of SEQ ID NO: 786. In some embodiments, the anti-CD3 antibody, or antigen-binding fragment thereof, comprises the VH of antibody ABL-CD3-99. The VH may comprise the amino acid sequence of SEQ ID NO: 1013. In some embodiments, the anti-CD3 antibody, or antigen-binding fragment thereof, comprises the VL and VH of antibody ABL-CD3-99. In some embodiments, the anti-CD3 antibody, or antigen-binding fragment thereof, comprises a VL comprising the amino acid sequence of SEQ ID NO: 786 and a VH comprising the amino acid sequence of SEQ ID NO: 1013. In some embodiments, the anti-CD3 antibody, or antigen-binding fragment thereof, comprises the VL of antibody ABL-CD3-99A. The VL may comprise the amino acid sequence of SEQ ID NO: 900. In some embodiments, the anti-CD3 antibody, or antigen-binding fragment thereof, comprises the VH of antibody ABL-CD3-99A. The VH may comprise the amino acid sequence of SEQ ID NO: 1127. In some embodiments, the anti-CD3 antibody, or antigen-binding fragment thereof, comprises the VL and VH of antibody ABL-CD3-99A. In some embodiments, the anti-CD3 antibody, or antigen-binding fragment thereof, comprises a VL comprising the amino acid sequence of SEQ ID NO: 900 and a VH comprising the amino acid sequence of SEQ ID NO: 1127. In some embodiments, the anti-CD3 antibody, or antigen-binding fragment thereof, comprises ABL-CD3-99_scFv_VH-VL. In some embodiments, the anti-CD3 antibody, or antigen-binding fragment thereof, comprises, from N-terminus to C-terminus, a VH comprising the amino acid sequence of SEQ ID NO: 1013; a linker comprising the amino acid sequence of (Gly-Gly-Gly-Gly-Ser)n (SEQ ID NO: 1146), wherein n is 4; and a VL comprising the amino acid sequence of SEQ ID NO: 786. In some embodiments, the anti-CD3 antibody, or antigen-binding fragment thereof, comprises ABL-CD3-99_scFv_VL-VH. In some embodiments, the anti-CD3 antibody, or antigen-binding fragment thereof, comprises, from N-terminus to C-terminus, a VL comprising the amino acid sequence of SEQ ID NO: 786; a linker comprising the amino acid sequence of (Gly-Gly-Gly-Gly-Ser)n (SEQ ID NO: 1146), wherein n is 4; and a VH comprising the amino acid sequence of SEQ ID NO: 1013. In some embodiments, the anti-CD3 antibody, or antigen-binding fragment thereof, comprises ABL-CD3-99A_scFv_VH-VL. In some embodiments, the anti-CD3 antibody, or antigen-binding fragment thereof, comprises, from N-terminus to C-terminus, a VH comprising the amino acid sequence of SEQ ID NO: 1127; a linker comprising the amino acid sequence of (Gly-Gly-Gly-Gly-Ser)n (SEQ ID NO: 1146), wherein n is 4; and a VL comprising the amino acid sequence of SEQ ID NO: 900. In some embodiments, the anti-CD3 antibody, or antigen-binding fragment thereof, comprises ABL-CD3-99A_scFv_VL-VH. In some embodiments, the anti-CD3 antibody, or antigen-binding fragment thereof, comprises, from N-terminus to C-terminus, a VL comprising the amino acid sequence of SEQ ID NO: 900; a linker comprising the amino acid sequence of (Gly-Gly-Gly-Gly-Ser)n (SEQ ID NO: 1146), wherein n is 4; and a VH comprising the amino acid sequence of SEQ ID NO: 1127.

In some embodiments, the anti-CD3 antibody, or antigen-binding fragment thereof, comprises the six CDRs of antibody ABL-CD3-100 and ABL-CD3-100A. The anti-CD3 antibody, or antigen-binding fragment thereof, may comprise an LCDR1, an LCDR2, and an LCDR3 comprising the amino acid sequences of SEQ ID NOs: 100, 213, and 326, respectively; and an HCDR1, an HCDR2, and an HCDR3 comprising the amino acid sequences of SEQ ID NOs: 444, 557, and 673, respectively. In some embodiments, the anti-CD3 antibody, or antigen-binding fragment thereof, comprises the VL of antibody ABL-CD3-100. The VL may comprise the amino acid sequence of SEQ ID NO: 787. In some embodiments, the anti-CD3 antibody, or antigen-binding fragment thereof, comprises the VH of antibody ABL-CD3-100. The VH may comprise the amino acid sequence of SEQ ID NO: 1014. In some embodiments, the anti-CD3 antibody, or antigen-binding fragment thereof, comprises the VL and VH of antibody ABL-CD3-100. In some embodiments, the anti-CD3 antibody, or antigen-binding fragment thereof, comprises a VL comprising the amino acid sequence of SEQ ID NO: 787 and a VH comprising the amino acid sequence of SEQ ID NO: 1014. In some embodiments, the anti-CD3 antibody, or antigen-binding fragment thereof, comprises the VL of antibody ABL-CD3-100A. The VL may comprise the amino acid sequence of SEQ ID NO: 901. In some embodiments, the anti-CD3 antibody, or antigen-binding fragment thereof, comprises the VH of antibody ABL-CD3-100A. The VH may comprise the amino acid sequence of SEQ ID NO: 1128. In some embodiments, the anti-CD3 antibody, or antigen-binding fragment thereof, comprises the VL and VH of antibody ABL-CD3-100A. In some embodiments, the anti-CD3 antibody, or antigen-binding fragment thereof, comprises a VL comprising the amino acid sequence of SEQ ID NO: 901 and a VH comprising the amino acid sequence of SEQ ID NO: 1128. In some embodiments, the anti-CD3 antibody, or antigen-binding fragment thereof, comprises ABL-CD3-100_scFv_VH-VL. In some embodiments, the anti-CD3 antibody, or antigen-binding fragment thereof, comprises, from N-terminus to C-terminus, a VH comprising the amino acid sequence of SEQ ID NO: 1014; a linker comprising the amino acid sequence of (Gly-Gly-Gly-Gly-Ser)n (SEQ ID NO: 1146), wherein n is 4; and a VL comprising the amino acid sequence of SEQ ID NO: 787. In some embodiments, the anti-CD3 antibody, or antigen-binding fragment thereof, comprises ABL-CD3-100_scFv_VL-VH. In some embodiments, the anti-CD3 antibody, or antigen-binding fragment thereof, comprises, from N-terminus to C-terminus, a VL comprising the amino acid sequence of SEQ ID NO: 787; a linker comprising the amino acid sequence of (Gly-Gly-Gly-Gly-Ser)n (SEQ ID NO: 1146), wherein n is 4; and a VH comprising the amino acid sequence of SEQ ID NO: 1014. In some embodiments, the anti-CD3 antibody, or antigen-binding fragment thereof, comprises ABL-CD3-100A_scFv_VH-VL. In some embodiments, the anti-CD3 antibody, or antigen-binding fragment thereof, comprises, from N-terminus to C-terminus, a VH comprising the amino acid sequence of SEQ ID NO: 1128; a linker comprising the amino acid sequence of (Gly-Gly-Gly-Gly-Ser)n (SEQ ID NO: 1146), wherein n is 4; and a VL comprising the amino acid sequence of SEQ ID NO: 901. In some embodiments, the anti-CD3 antibody, or antigen-binding fragment thereof, comprises ABL-CD3-100A_scFv_VL-VH. In some embodiments, the anti-CD3 antibody, or antigen-binding fragment thereof, comprises, from N-terminus to C-terminus, a VL comprising the amino acid sequence of SEQ ID NO: 901; a linker comprising the amino acid sequence of (Gly-Gly-Gly-Gly-Ser)n (SEQ ID NO: 1146), wherein n is 4; and a VH comprising the amino acid sequence of SEQ ID NO: 1128.

In some embodiments, the anti-CD3 antibody, or antigen-binding fragment thereof, comprises the six CDRs of antibody ABL-CD3-101 and ABL-CD3-101A. The anti-CD3 antibody, or antigen-binding fragment thereof, may comprise an LCDR1, an LCDR2, and an LCDR3 comprising the amino acid sequences of SEQ ID NOs: 101, 214, and 327, respectively; and an HCDR1, an HCDR2, and an HCDR3 comprising the amino acid sequences of SEQ ID NOs: 445, 558, and 674, respectively. In some embodiments, the anti-CD3 antibody, or antigen-binding fragment thereof, comprises the VL of antibody ABL-CD3-101. The VL may comprise the amino acid sequence of SEQ ID NO: 788. In some embodiments, the anti-CD3 antibody, or antigen-binding fragment thereof, comprises the VH of antibody ABL-CD3-101. The VH may comprise the amino acid sequence of SEQ ID NO: 1015. In some embodiments, the anti-CD3 antibody, or antigen-binding fragment thereof, comprises the VL and VH of antibody ABL-CD3-101. In some embodiments, the anti-CD3 antibody, or antigen-binding fragment thereof, comprises a VL comprising the amino acid sequence of SEQ ID NO: 788 and a VH comprising the amino acid sequence of SEQ ID NO: 1015. In some embodiments, the anti-CD3 antibody, or antigen-binding fragment thereof, comprises the VL of antibody ABL-CD3-101A. The VL may comprise the amino acid sequence of SEQ ID NO: 902. In some embodiments, the anti-CD3 antibody, or antigen-binding fragment thereof, comprises the VH of antibody ABL-CD3-101A. The VH may comprise the amino acid sequence of SEQ ID NO: 1129. In some embodiments, the anti-CD3 antibody, or antigen-binding fragment thereof, comprises the VL and VH of antibody ABL-CD3-101A. In some embodiments, the anti-CD3 antibody, or antigen-binding fragment thereof, comprises a VL comprising the amino acid sequence of SEQ ID NO: 902 and a VH comprising the amino acid sequence of SEQ ID NO: 1129. In some embodiments, the anti-CD3 antibody, or antigen-binding fragment thereof, comprises ABL-CD3-101_scFv_VH-VL. In some embodiments, the anti-CD3 antibody, or antigen-binding fragment thereof, comprises, from N-terminus to C-terminus, a VH comprising the amino acid sequence of SEQ ID NO: 1015; a linker comprising the amino acid sequence of (Gly-Gly-Gly-Gly-Ser)n (SEQ ID NO: 1146), wherein n is 4; and a VL comprising the amino acid sequence of SEQ ID NO: 788. In some embodiments, the anti-CD3 antibody, or antigen-binding fragment thereof, comprises ABL-CD3-101_scFv_VL-VH. In some embodiments, the anti-CD3 antibody, or antigen-binding fragment thereof, comprises, from N-terminus to C-terminus, a VL comprising the amino acid sequence of SEQ ID NO: 788; a linker comprising the amino acid sequence of (Gly-Gly-Gly-Gly-Ser)n (SEQ ID NO: 1146), wherein n is 4; and a VH comprising the amino acid sequence of SEQ ID NO: 1015. In some embodiments, the anti-CD3 antibody, or antigen-binding fragment thereof, comprises ABL-CD3-101A_scFv_VH-VL. In some embodiments, the anti-CD3 antibody, or antigen-binding fragment thereof, comprises, from N-terminus to C-terminus, a VH comprising the amino acid sequence of SEQ ID NO: 1129; a linker comprising the amino acid sequence of (Gly-Gly-Gly-Gly-Ser)n (SEQ ID NO: 1146), wherein n is 4; and a VL comprising the amino acid sequence of SEQ ID NO: 902. In some embodiments, the anti-CD3 antibody, or antigen-binding fragment thereof, comprises ABL-CD3-101A_scFv_VL-VH. In some embodiments, the anti-CD3 antibody, or antigen-binding fragment thereof, comprises, from N-terminus to C-terminus, a VL comprising the amino acid sequence of SEQ ID NO: 902; a linker comprising the amino acid sequence of (Gly-Gly-Gly-Gly-Ser)n (SEQ ID NO: 1146), wherein n is 4; and a VH comprising the amino acid sequence of SEQ ID NO: 1129.

In some embodiments, the anti-CD3 antibody, or antigen-binding fragment thereof, comprises the six CDRs of antibody ABL-CD3-102 and ABL-CD3-102A. The anti-CD3 antibody, or antigen-binding fragment thereof, may comprise an LCDR1, an LCDR2, and an LCDR3 comprising the amino acid sequences of SEQ ID NOs: 102, 215, and 328, respectively; and an HCDR1, an HCDR2, and an HCDR3 comprising the amino acid sequences of SEQ ID NOs: 446, 559, and 675, respectively. In some embodiments, the anti-CD3 antibody, or antigen-binding fragment thereof, comprises the VL of antibody ABL-CD3-102. The VL may comprise the amino acid sequence of SEQ ID NO: 789. In some embodiments, the anti-CD3 antibody, or antigen-binding fragment thereof, comprises the VH of antibody ABL-CD3-102. The VH may comprise the amino acid sequence of SEQ ID NO: 1016. In some embodiments, the anti-CD3 antibody, or antigen-binding fragment thereof, comprises the VL and VH of antibody ABL-CD3-102. In some embodiments, the anti-CD3 antibody, or antigen-binding fragment thereof, comprises a VL comprising the amino acid sequence of SEQ ID NO: 789 and a VH comprising the amino acid sequence of SEQ ID NO: 1016. In some embodiments, the anti-CD3 antibody, or antigen-binding fragment thereof, comprises the VL of antibody ABL-CD3-102A. The VL may comprise the amino acid sequence of SEQ ID NO: 903. In some embodiments, the anti-CD3 antibody, or antigen-binding fragment thereof, comprises the VH of antibody ABL-CD3-102A. The VH may comprise the amino acid sequence of SEQ ID NO: 1130. In some embodiments, the anti-CD3 antibody, or antigen-binding fragment thereof, comprises the VL and VH of antibody ABL-CD3-102A. In some embodiments, the anti-CD3 antibody, or antigen-binding fragment thereof, comprises a VL comprising the amino acid sequence of SEQ ID NO: 903 and a VH comprising the amino acid sequence of SEQ ID NO: 1130. In some embodiments, the anti-CD3 antibody, or antigen-binding fragment thereof, comprises ABL-CD3-102_scFv_VH-VL. In some embodiments, the anti-CD3 antibody, or antigen-binding fragment thereof, comprises, from N-terminus to C-terminus, a VH comprising the amino acid sequence of SEQ ID NO: 1016; a linker comprising the amino acid sequence of (Gly-Gly-Gly-Gly-Ser)n (SEQ ID NO: 1146), wherein n is 4; and a VL comprising the amino acid sequence of SEQ ID NO: 789. In some embodiments, the anti-CD3 antibody, or antigen-binding fragment thereof, comprises ABL-CD3-102_scFv_VL-VH. In some embodiments, the anti-CD3 antibody, or antigen-binding fragment thereof, comprises, from N-terminus to C-terminus, a VL comprising the amino acid sequence of SEQ ID NO: 789; a linker comprising the amino acid sequence of (Gly-Gly-Gly-Gly-Ser)n (SEQ ID NO: 1146), wherein n is 4; and a VH comprising the amino acid sequence of SEQ ID NO: 1016. In some embodiments, the anti-CD3 antibody, or antigen-binding fragment thereof, comprises ABL-CD3-102A_scFv_VH-VL. In some embodiments, the anti-CD3 antibody, or antigen-binding fragment thereof, comprises, from N-terminus to C-terminus, a VH comprising the amino acid sequence of SEQ ID NO: 1130; a linker comprising the amino acid sequence of (Gly-Gly-Gly-Gly-Ser)n (SEQ ID NO: 1146), wherein n is 4; and a VL comprising the amino acid sequence of SEQ ID NO: 903. In some embodiments, the anti-CD3 antibody, or antigen-binding fragment thereof, comprises ABL-CD3-102A_scFv_VL-VH. In some embodiments, the anti-CD3 antibody, or antigen-binding fragment thereof, comprises, from N-terminus to C-terminus, a VL comprising the amino acid sequence of SEQ ID NO: 903; a linker comprising the amino acid sequence of (Gly-Gly-Gly-Gly-Ser)n (SEQ ID NO: 1146), wherein n is 4; and a VH comprising the amino acid sequence of SEQ ID NO: 1130.

In some embodiments, the anti-CD3 antibody, or antigen-binding fragment thereof, comprises the six CDRs of antibody ABL-CD3-103 and ABL-CD3-103A. The anti-CD3 antibody, or antigen-binding fragment thereof, may comprise an LCDR1, an LCDR2, and an LCDR3 comprising the amino acid sequences of SEQ ID NOs: 103, 216, and 329, respectively; and an HCDR1, an HCDR2, and an HCDR3 comprising the amino acid sequences of SEQ ID NOs: 447, 560, and 676, respectively. In some embodiments, the anti-CD3 antibody, or antigen-binding fragment thereof, comprises the VL of antibody ABL-CD3-103. The VL may comprise the amino acid sequence of SEQ ID NO: 790. In some embodiments, the anti-CD3 antibody, or antigen-binding fragment thereof, comprises the VH of antibody ABL-CD3-103. The VH may comprise the amino acid sequence of SEQ ID NO: 1017. In some embodiments, the anti-CD3 antibody, or antigen-binding fragment thereof, comprises the VL and VH of antibody ABL-CD3-103. In some embodiments, the anti-CD3 antibody, or antigen-binding fragment thereof, comprises a VL comprising the amino acid sequence of SEQ ID NO: 790 and a VH comprising the amino acid sequence of SEQ ID NO: 1017. In some embodiments, the anti-CD3 antibody, or antigen-binding fragment thereof, comprises the VL of antibody ABL-CD3-103A. The VL may comprise the amino acid sequence of SEQ ID NO: 904. In some embodiments, the anti-CD3 antibody, or antigen-binding fragment thereof, comprises the VH of antibody ABL-CD3-103A. The VH may comprise the amino acid sequence of SEQ ID NO: 1131. In some embodiments, the anti-CD3 antibody, or antigen-binding fragment thereof, comprises the VL and VH of antibody ABL-CD3-103A. In some embodiments, the anti-CD3 antibody, or antigen-binding fragment thereof, comprises a VL comprising the amino acid sequence of SEQ ID NO: 904 and a VH comprising the amino acid sequence of SEQ ID NO: 1131. In some embodiments, the anti-CD3 antibody, or antigen-binding fragment thereof, comprises ABL-CD3-103_scFv_VH-VL. In some embodiments, the anti-CD3 antibody, or antigen-binding fragment thereof, comprises, from N-terminus to C-terminus, a VH comprising the amino acid sequence of SEQ ID NO: 1017; a linker comprising the amino acid sequence of (Gly-Gly-Gly-Gly-Ser)n (SEQ ID NO: 1146), wherein n is 4; and a VL comprising the amino acid sequence of SEQ ID NO: 790. In some embodiments, the anti-CD3 antibody, or antigen-binding fragment thereof, comprises ABL-CD3-103_scFv_VL-VH. In some embodiments, the anti-CD3 antibody, or antigen-binding fragment thereof, comprises, from N-terminus to C-terminus, a VL comprising the amino acid sequence of SEQ ID NO: 790; a linker comprising the amino acid sequence of (Gly-Gly-Gly-Gly-Ser)n (SEQ ID NO: 1146), wherein n is 4; and a VH comprising the amino acid sequence of SEQ ID NO: 1017. In some embodiments, the anti-CD3 antibody, or antigen-binding fragment thereof, comprises ABL-CD3-103A_scFv_VH-VL. In some embodiments, the anti-CD3 antibody, or antigen-binding fragment thereof, comprises, from N-terminus to C-terminus, a VH comprising the amino acid sequence of SEQ ID NO: 1131; a linker comprising the amino acid sequence of (Gly-Gly-Gly-Gly-Ser)n (SEQ ID NO: 1146), wherein n is 4; and a VL comprising the amino acid sequence of SEQ ID NO: 904. In some embodiments, the anti-CD3 antibody, or antigen-binding fragment thereof, comprises ABL-CD3-103A_scFv_VL-VH. In some embodiments, the anti-CD3 antibody, or antigen-binding fragment thereof, comprises, from N-terminus to C-terminus, a VL comprising the amino acid sequence of SEQ ID NO: 904; a linker comprising the amino acid sequence of (Gly-Gly-Gly-Gly-Ser)n (SEQ ID NO: 1146), wherein n is 4; and a VH comprising the amino acid sequence of SEQ ID NO: 1131.

In some embodiments, the anti-CD3 antibody, or antigen-binding fragment thereof, comprises the six CDRs of antibody ABL-CD3-104 and ABL-CD3-104A. The anti-CD3 antibody, or antigen-binding fragment thereof, may comprise an LCDR1, an LCDR2, and an LCDR3 comprising the amino acid sequences of SEQ ID NOs: 104, 217, and 330, respectively; and an HCDR1, an HCDR2, and an HCDR3 comprising the amino acid sequences of SEQ ID NOs: 448, 561, and 677, respectively. In some embodiments, the anti-CD3 antibody, or antigen-binding fragment thereof, comprises the VL of antibody ABL-CD3-104. The VL may comprise the amino acid sequence of SEQ ID NO: 791. In some embodiments, the anti-CD3 antibody, or antigen-binding fragment thereof, comprises the VH of antibody ABL-CD3-104. The VH may comprise the amino acid sequence of SEQ ID NO: 1018. In some embodiments, the anti-CD3 antibody, or antigen-binding fragment thereof, comprises the VL and VH of antibody ABL-CD3-104. In some embodiments, the anti-CD3 antibody, or antigen-binding fragment thereof, comprises a VL comprising the amino acid sequence of SEQ ID NO: 791 and a VH comprising the amino acid sequence of SEQ ID NO: 1018. In some embodiments, the anti-CD3 antibody, or antigen-binding fragment thereof, comprises the VL of antibody ABL-CD3-104A. The VL may comprise the amino acid sequence of SEQ ID NO: 905. In some embodiments, the anti-CD3 antibody, or antigen-binding fragment thereof, comprises the VH of antibody ABL-CD3-104A. The VH may comprise the amino acid sequence of SEQ ID NO: 1132. In some embodiments, the anti-CD3 antibody, or antigen-binding fragment thereof, comprises the VL and VH of antibody ABL-CD3-104A. In some embodiments, the anti-CD3 antibody, or antigen-binding fragment thereof, comprises a VL comprising the amino acid sequence of SEQ ID NO: 905 and a VH comprising the amino acid sequence of SEQ ID NO: 1132. In some embodiments, the anti-CD3 antibody, or antigen-binding fragment thereof, comprises ABL-CD3-104_scFv_VH-VL. In some embodiments, the anti-CD3 antibody, or antigen-binding fragment thereof, comprises, from N-terminus to C-terminus, a VH comprising the amino acid sequence of SEQ ID NO: 1018; a linker comprising the amino acid sequence of (Gly-Gly-Gly-Gly-Ser)n (SEQ ID NO: 1146), wherein n is 4; and a VL comprising the amino acid sequence of SEQ ID NO: 791. In some embodiments, the anti-CD3 antibody, or antigen-binding fragment thereof, comprises ABL-CD3-104_scFv_VL-VH. In some embodiments, the anti-CD3 antibody, or antigen-binding fragment thereof, comprises, from N-terminus to C-terminus, a VL comprising the amino acid sequence of SEQ ID NO: 791; a linker comprising the amino acid sequence of (Gly-Gly-Gly-Gly-Ser)n (SEQ ID NO: 1146), wherein n is 4; and a VH comprising the amino acid sequence of SEQ ID NO: 1018. In some embodiments, the anti-CD3 antibody, or antigen-binding fragment thereof, comprises ABL-CD3-104A_scFv_VH-VL. In some embodiments, the anti-CD3 antibody, or antigen-binding fragment thereof, comprises, from N-terminus to C-terminus, a VH comprising the amino acid sequence of SEQ ID NO: 1132; a linker comprising the amino acid sequence of (Gly-Gly-Gly-Gly-Ser)n (SEQ ID NO: 1146), wherein n is 4; and a VL comprising the amino acid sequence of SEQ ID NO: 905. In some embodiments, the anti-CD3 antibody, or antigen-binding fragment thereof, comprises ABL-CD3-104A_scFv_VL-VH. In some embodiments, the anti-CD3 antibody, or antigen-binding fragment thereof, comprises, from N-terminus to C-terminus, a VL comprising the amino acid sequence of SEQ ID NO: 905; a linker comprising the amino acid sequence of (Gly-Gly-Gly-Gly-Ser)n (SEQ ID NO: 1146), wherein n is 4; and a VH comprising the amino acid sequence of SEQ ID NO: 1132.

In some embodiments, the anti-CD3 antibody, or antigen-binding fragment thereof, comprises the six CDRs of antibody ABL-CD3-105 and ABL-CD3-105A. The anti-CD3 antibody, or antigen-binding fragment thereof, may comprise an LCDR1, an LCDR2, and an LCDR3 comprising the amino acid sequences of SEQ ID NOs: 105, 218, and 331, respectively; and an HCDR1, an HCDR2, and an HCDR3 comprising the amino acid sequences of SEQ ID NOs: 449, 562, and 678, respectively. In some embodiments, the anti-CD3 antibody, or antigen-binding fragment thereof, comprises the VL of antibody ABL-CD3-105. The VL may comprise the amino acid sequence of SEQ ID NO: 792. In some embodiments, the anti-CD3 antibody, or antigen-binding fragment thereof, comprises the VH of antibody ABL-CD3-105. The VH may comprise the amino acid sequence of SEQ ID NO: 1019. In some embodiments, the anti-CD3 antibody, or antigen-binding fragment thereof, comprises the VL and VH of antibody ABL-CD3-105. In some embodiments, the anti-CD3 antibody, or antigen-binding fragment thereof, comprises a VL comprising the amino acid sequence of SEQ ID NO: 792 and a VH comprising the amino acid sequence of SEQ ID NO: 1019. In some embodiments, the anti-CD3 antibody, or antigen-binding fragment thereof, comprises the VL of antibody ABL-CD3-105A. The VL may comprise the amino acid sequence of SEQ ID NO: 906. In some embodiments, the anti-CD3 antibody, or antigen-binding fragment thereof, comprises the VH of antibody ABL-CD3-105A. The VH may comprise the amino acid sequence of SEQ ID NO: 1133. In some embodiments, the anti-CD3 antibody, or antigen-binding fragment thereof, comprises the VL and VH of antibody ABL-CD3-105A. In some embodiments, the anti-CD3 antibody, or antigen-binding fragment thereof, comprises a VL comprising the amino acid sequence of SEQ ID NO: 906 and a VH comprising the amino acid sequence of SEQ ID NO: 1133. In some embodiments, the anti-CD3 antibody, or antigen-binding fragment thereof, comprises ABL-CD3-105_scFv_VH-VL. In some embodiments, the anti-CD3 antibody, or antigen-binding fragment thereof, comprises, from N-terminus to C-terminus, a VH comprising the amino acid sequence of SEQ ID NO: 1019; a linker comprising the amino acid sequence of (Gly-Gly-Gly-Gly-Ser)n (SEQ ID NO: 1146), wherein n is 4; and a VL comprising the amino acid sequence of SEQ ID NO: 792. In some embodiments, the anti-CD3 antibody, or antigen-binding fragment thereof, comprises ABL-CD3-105_scFv_VL-VH. In some embodiments, the anti-CD3 antibody, or antigen-binding fragment thereof, comprises, from N-terminus to C-terminus, a VL comprising the amino acid sequence of SEQ ID NO: 792; a linker comprising the amino acid sequence of (Gly-Gly-Gly-Gly-Ser)n (SEQ ID NO: 1146), wherein n is 4; and a VH comprising the amino acid sequence of SEQ ID NO: 1019. In some embodiments, the anti-CD3 antibody, or antigen-binding fragment thereof, comprises ABL-CD3-105A_scFv_VH-VL. In some embodiments, the anti-CD3 antibody, or antigen-binding fragment thereof, comprises, from N-terminus to C-terminus, a VH comprising the amino acid sequence of SEQ ID NO: 1133; a linker comprising the amino acid sequence of (Gly-Gly-Gly-Gly-Ser)n (SEQ ID NO: 1146), wherein n is 4; and a VL comprising the amino acid sequence of SEQ ID NO: 906. In some embodiments, the anti-CD3 antibody, or antigen-binding fragment thereof, comprises ABL-CD3-105A_scFv_VL-VH. In some embodiments, the anti-CD3 antibody, or antigen-binding fragment thereof, comprises, from N-terminus to C-terminus, a VL comprising the amino acid sequence of SEQ ID NO: 906; a linker comprising the amino acid sequence of (Gly-Gly-Gly-Gly-Ser)n (SEQ ID NO: 1146), wherein n is 4; and a VH comprising the amino acid sequence of SEQ ID NO: 1133.

In some embodiments, the anti-CD3 antibody, or antigen-binding fragment thereof, comprises the six CDRs of antibody ABL-CD3-106 and ABL-CD3-106A. The anti-CD3 antibody, or antigen-binding fragment thereof, may comprise an LCDR1, an LCDR2, and an LCDR3 comprising the amino acid sequences of SEQ ID NOs: 106, 219, and 332, respectively; and an HCDR1, an HCDR2, and an HCDR3 comprising the amino acid sequences of SEQ ID NOs: 450, 563, and 679, respectively. In some embodiments, the anti-CD3 antibody, or antigen-binding fragment thereof, comprises the VL of antibody ABL-CD3-106. The VL may comprise the amino acid sequence of SEQ ID NO: 793. In some embodiments, the anti-CD3 antibody, or antigen-binding fragment thereof, comprises the VH of antibody ABL-CD3-106. The VH may comprise the amino acid sequence of SEQ ID NO: 1020. In some embodiments, the anti-CD3 antibody, or antigen-binding fragment thereof, comprises the VL and VH of antibody ABL-CD3-106. In some embodiments, the anti-CD3 antibody, or antigen-binding fragment thereof, comprises a VL comprising the amino acid sequence of SEQ ID NO: 793 and a VH comprising the amino acid sequence of SEQ ID NO: 1020. In some embodiments, the anti-CD3 antibody, or antigen-binding fragment thereof, comprises the VL of antibody ABL-CD3-106A. The VL may comprise the amino acid sequence of SEQ ID NO: 907. In some embodiments, the anti-CD3 antibody, or antigen-binding fragment thereof, comprises the VH of antibody ABL-CD3-106A. The VH may comprise the amino acid sequence of SEQ ID NO: 1134. In some embodiments, the anti-CD3 antibody, or antigen-binding fragment thereof, comprises the VL and VH of antibody ABL-CD3-106A. In some embodiments, the anti-CD3 antibody, or antigen-binding fragment thereof, comprises a VL comprising the amino acid sequence of SEQ ID NO: 907 and a VH comprising the amino acid sequence of SEQ ID NO: 1134. In some embodiments, the anti-CD3 antibody, or antigen-binding fragment thereof, comprises ABL-CD3-106_scFv_VH-VL. In some embodiments, the anti-CD3 antibody, or antigen-binding fragment thereof, comprises, from N-terminus to C-terminus, a VH comprising the amino acid sequence of SEQ ID NO: 1020; a linker comprising the amino acid sequence of (Gly-Gly-Gly-Gly-Ser)n (SEQ ID NO: 1146), wherein n is 4; and a VL comprising the amino acid sequence of SEQ ID NO: 793. In some embodiments, the anti-CD3 antibody, or antigen-binding fragment thereof, comprises ABL-CD3-106_scFv_VL-VH. In some embodiments, the anti-CD3 antibody, or antigen-binding fragment thereof, comprises, from N-terminus to C-terminus, a VL comprising the amino acid sequence of SEQ ID NO: 793; a linker comprising the amino acid sequence of (Gly-Gly-Gly-Gly-Ser)n (SEQ ID NO: 1146), wherein n is 4; and a VH comprising the amino acid sequence of SEQ ID NO: 1020. In some embodiments, the anti-CD3 antibody, or antigen-binding fragment thereof, comprises ABL-CD3-106A_scFv_VH-VL. In some embodiments, the anti-CD3 antibody, or antigen-binding fragment thereof, comprises, from N-terminus to C-terminus, a VH comprising the amino acid sequence of SEQ ID NO: 1134; a linker comprising the amino acid sequence of (Gly-Gly-Gly-Gly-Ser)n (SEQ ID NO: 1146), wherein n is 4; and a VL comprising the amino acid sequence of SEQ ID NO: 907. In some embodiments, the anti-CD3 antibody, or antigen-binding fragment thereof, comprises ABL-CD3-106A_scFv_VL-VH. In some embodiments, the anti-CD3 antibody, or antigen-binding fragment thereof, comprises, from N-terminus to C-terminus, a VL comprising the amino acid sequence of SEQ ID NO: 907; a linker comprising the amino acid sequence of (Gly-Gly-Gly-Gly-Ser)n (SEQ ID NO: 1146), wherein n is 4; and a VH comprising the amino acid sequence of SEQ ID NO: 1134.

In some embodiments, the anti-CD3 antibody, or antigen-binding fragment thereof, comprises the six CDRs of antibody ABL-CD3-107 and ABL-CD3-107A. The anti-CD3 antibody, or antigen-binding fragment thereof, may comprise an LCDR1, an LCDR2, and an LCDR3 comprising the amino acid sequences of SEQ ID NOs: 107, 220, and 333, respectively; and an HCDR1, an HCDR2, and an HCDR3 comprising the amino acid sequences of SEQ ID NOs: 451, 564, and 680, respectively. In some embodiments, the anti-CD3 antibody, or antigen-binding fragment thereof, comprises the VL of antibody ABL-CD3-107. The VL may comprise the amino acid sequence of SEQ ID NO: 794. In some embodiments, the anti-CD3 antibody, or antigen-binding fragment thereof, comprises the VH of antibody ABL-CD3-107. The VH may comprise the amino acid sequence of SEQ ID NO: 1021. In some embodiments, the anti-CD3 antibody, or antigen-binding fragment thereof, comprises the VL and VH of antibody ABL-CD3-107. In some embodiments, the anti-CD3 antibody, or antigen-binding fragment thereof, comprises a VL comprising the amino acid sequence of SEQ ID NO: 794 and a VH comprising the amino acid sequence of SEQ ID NO: 1021. In some embodiments, the anti-CD3 antibody, or antigen-binding fragment thereof, comprises the VL of antibody ABL-CD3-107A. The VL may comprise the amino acid sequence of SEQ ID NO: 908. In some embodiments, the anti-CD3 antibody, or antigen-binding fragment thereof, comprises the VH of antibody ABL-CD3-107A. The VH may comprise the amino acid sequence of SEQ ID NO: 1135. In some embodiments, the anti-CD3 antibody, or antigen-binding fragment thereof, comprises the VL and VH of antibody ABL-CD3-107A. In some embodiments, the anti-CD3 antibody, or antigen-binding fragment thereof, comprises a VL comprising the amino acid sequence of SEQ ID NO: 908 and a VH comprising the amino acid sequence of SEQ ID NO: 1135. In some embodiments, the anti-CD3 antibody, or antigen-binding fragment thereof, comprises ABL-CD3-107_scFv_VH-VL. In some embodiments, the anti-CD3 antibody, or antigen-binding fragment thereof, comprises, from N-terminus to C-terminus, a VH comprising the amino acid sequence of SEQ ID NO: 1021; a linker comprising the amino acid sequence of (Gly-Gly-Gly-Gly-Ser)n (SEQ ID NO: 1146), wherein n is 4; and a VL comprising the amino acid sequence of SEQ ID NO: 794. In some embodiments, the anti-CD3 antibody, or antigen-binding fragment thereof, comprises ABL-CD3-107_scFv_VL-VH. In some embodiments, the anti-CD3 antibody, or antigen-binding fragment thereof, comprises, from N-terminus to C-terminus, a VL comprising the amino acid sequence of SEQ ID NO: 794; a linker comprising the amino acid sequence of (Gly-Gly-Gly-Gly-Ser)n (SEQ ID NO: 1146), wherein n is 4; and a VH comprising the amino acid sequence of SEQ ID NO: 1021. In some embodiments, the anti-CD3 antibody, or antigen-binding fragment thereof, comprises ABL-CD3-107A_scFv_VH-VL. In some embodiments, the anti-CD3 antibody, or antigen-binding fragment thereof, comprises, from N-terminus to C-terminus, a VH comprising the amino acid sequence of SEQ ID NO: 1135; a linker comprising the amino acid sequence of (Gly-Gly-Gly-Gly-Ser)n (SEQ ID NO: 1146), wherein n is 4; and a VL comprising the amino acid sequence of SEQ ID NO: 908. In some embodiments, the anti-CD3 antibody, or antigen-binding fragment thereof, comprises ABL-CD3-107A_scFv_VL-VH. In some embodiments, the anti-CD3 antibody, or antigen-binding fragment thereof, comprises, from N-terminus to C-terminus, a VL comprising the amino acid sequence of SEQ ID NO: 908; a linker comprising the amino acid sequence of (Gly-Gly-Gly-Gly-Ser)n (SEQ ID NO: 1146), wherein n is 4; and a VH comprising the amino acid sequence of SEQ ID NO: 1135.

In some embodiments, the anti-CD3 antibody, or antigen-binding fragment thereof, comprises the six CDRs of antibody ABL-CD3-108 and ABL-CD3-108A. The anti-CD3 antibody, or antigen-binding fragment thereof, may comprise an LCDR1, an LCDR2, and an LCDR3 comprising the amino acid sequences of SEQ ID NOs: 108, 221, and 334, respectively; and an HCDR1, an HCDR2, and an HCDR3 comprising the amino acid sequences of SEQ ID NOs: 452, 565, and 681, respectively. In some embodiments, the anti-CD3 antibody, or antigen-binding fragment thereof, comprises the VL of antibody ABL-CD3-108. The VL may comprise the amino acid sequence of SEQ ID NO: 795. In some embodiments, the anti-CD3 antibody, or antigen-binding fragment thereof, comprises the VH of antibody ABL-CD3-108. The VH may comprise the amino acid sequence of SEQ ID NO: 1022. In some embodiments, the anti-CD3 antibody, or antigen-binding fragment thereof, comprises the VL and VH of antibody ABL-CD3-108. In some embodiments, the anti-CD3 antibody, or antigen-binding fragment thereof, comprises a VL comprising the amino acid sequence of SEQ ID NO: 795 and a VH comprising the amino acid sequence of SEQ ID NO: 1022. In some embodiments, the anti-CD3 antibody, or antigen-binding fragment thereof, comprises the VL of antibody ABL-CD3-108A. The VL may comprise the amino acid sequence of SEQ ID NO: 909. In some embodiments, the anti-CD3 antibody, or antigen-binding fragment thereof, comprises the VH of antibody ABL-CD3-108A. The VH may comprise the amino acid sequence of SEQ ID NO: 1136. In some embodiments, the anti-CD3 antibody, or antigen-binding fragment thereof, comprises the VL and VH of antibody ABL-CD3-108A. In some embodiments, the anti-CD3 antibody, or antigen-binding fragment thereof, comprises a VL comprising the amino acid sequence of SEQ ID NO: 909 and a VH comprising the amino acid sequence of SEQ ID NO: 1136. In some embodiments, the anti-CD3 antibody, or antigen-binding fragment thereof, comprises ABL-CD3-108_scFv_VH-VL. In some embodiments, the anti-CD3 antibody, or antigen-binding fragment thereof, comprises, from N-terminus to C-terminus, a VH comprising the amino acid sequence of SEQ ID NO: 1022; a linker comprising the amino acid sequence of (Gly-Gly-Gly-Gly-Ser)n (SEQ ID NO: 1146), wherein n is 4; and a VL comprising the amino acid sequence of SEQ ID NO: 795. In some embodiments, the anti-CD3 antibody, or antigen-binding fragment thereof, comprises ABL-CD3-108_scFv_VL-VH. In some embodiments, the anti-CD3 antibody, or antigen-binding fragment thereof, comprises, from N-terminus to C-terminus, a VL comprising the amino acid sequence of SEQ ID NO: 795; a linker comprising the amino acid sequence of (Gly-Gly-Gly-Gly-Ser)n (SEQ ID NO: 1146), wherein n is 4; and a VH comprising the amino acid sequence of SEQ ID NO: 1022. In some embodiments, the anti-CD3 antibody, or antigen-binding fragment thereof, comprises ABL-CD3-108A_scFv_VH-VL. In some embodiments, the anti-CD3 antibody, or antigen-binding fragment thereof, comprises, from N-terminus to C-terminus, a VH comprising the amino acid sequence of SEQ ID NO: 1136; a linker comprising the amino acid sequence of (Gly-Gly-Gly-Gly-Ser)n (SEQ ID NO: 1146), wherein n is 4; and a VL comprising the amino acid sequence of SEQ ID NO: 909. In some embodiments, the anti-CD3 antibody, or antigen-binding fragment thereof, comprises ABL-CD3-108A_scFv_VL-VH. In some embodiments, the anti-CD3 antibody, or antigen-binding fragment thereof, comprises, from N-terminus to C-terminus, a VL comprising the amino acid sequence of SEQ ID NO: 909; a linker comprising the amino acid sequence of (Gly-Gly-Gly-Gly-Ser)n (SEQ ID NO: 1146), wherein n is 4; and a VH comprising the amino acid sequence of SEQ ID NO: 1136.

In some embodiments, the anti-CD3 antibody, or antigen-binding fragment thereof, comprises the six CDRs of antibody ABL-CD3-109 and ABL-CD3-109A. The anti-CD3 antibody, or antigen-binding fragment thereof, may comprise an LCDR1, an LCDR2, and an LCDR3 comprising the amino acid sequences of SEQ ID NOs: 109, 222, and 335, respectively; and an HCDR1, an HCDR2, and an HCDR3 comprising the amino acid sequences of SEQ ID NOs: 453, 566, and 682, respectively. In some embodiments, the anti-CD3 antibody, or antigen-binding fragment thereof, comprises the VL of antibody ABL-CD3-109. The VL may comprise the amino acid sequence of SEQ ID NO: 796. In some embodiments, the anti-CD3 antibody, or antigen-binding fragment thereof, comprises the VH of antibody ABL-CD3-109. The VH may comprise the amino acid sequence of SEQ ID NO: 1023. In some embodiments, the anti-CD3 antibody, or antigen-binding fragment thereof, comprises the VL and VH of antibody ABL-CD3-109. In some embodiments, the anti-CD3 antibody, or antigen-binding fragment thereof, comprises a VL comprising the amino acid sequence of SEQ ID NO: 796 and a VH comprising the amino acid sequence of SEQ ID NO: 1023. In some embodiments, the anti-CD3 antibody, or antigen-binding fragment thereof, comprises the VL of antibody ABL-CD3-109A. The VL may comprise the amino acid sequence of SEQ ID NO: 910. In some embodiments, the anti-CD3 antibody, or antigen-binding fragment thereof, comprises the VH of antibody ABL-CD3-109A. The VH may comprise the amino acid sequence of SEQ ID NO: 1137. In some embodiments, the anti-CD3 antibody, or antigen-binding fragment thereof, comprises the VL and VH of antibody ABL-CD3-109A. In some embodiments, the anti-CD3 antibody, or antigen-binding fragment thereof, comprises a VL comprising the amino acid sequence of SEQ ID NO: 910 and a VH comprising the amino acid sequence of SEQ ID NO: 1137. In some embodiments, the anti-CD3 antibody, or antigen-binding fragment thereof, comprises ABL-CD3-109_scFv_VH-VL. In some embodiments, the anti-CD3 antibody, or antigen-binding fragment thereof, comprises, from N-terminus to C-terminus, a VH comprising the amino acid sequence of SEQ ID NO: 1023; a linker comprising the amino acid sequence of (Gly-Gly-Gly-Gly-Ser)n (SEQ ID NO: 1146), wherein n is 4; and a VL comprising the amino acid sequence of SEQ ID NO: 796. In some embodiments, the anti-CD3 antibody, or antigen-binding fragment thereof, comprises ABL-CD3-109_scFv_VL-VH. In some embodiments, the anti-CD3 antibody, or antigen-binding fragment thereof, comprises, from N-terminus to C-terminus, a VL comprising the amino acid sequence of SEQ ID NO: 796; a linker comprising the amino acid sequence of (Gly-Gly-Gly-Gly-Ser)n (SEQ ID NO: 1146), wherein n is 4; and a VH comprising the amino acid sequence of SEQ ID NO: 1023. In some embodiments, the anti-CD3 antibody, or antigen-binding fragment thereof, comprises ABL-CD3-109A_scFv_VH-VL. In some embodiments, the anti-CD3 antibody, or antigen-binding fragment thereof, comprises, from N-terminus to C-terminus, a VH comprising the amino acid sequence of SEQ ID NO: 1137; a linker comprising the amino acid sequence of (Gly-Gly-Gly-Gly-Ser)n (SEQ ID NO: 1146), wherein n is 4; and a VL comprising the amino acid sequence of SEQ ID NO: 910. In some embodiments, the anti-CD3 antibody, or antigen-binding fragment thereof, comprises ABL-CD3-109A_scFv_VL-VH. In some embodiments, the anti-CD3 antibody, or antigen-binding fragment thereof, comprises, from N-terminus to C-terminus, a VL comprising the amino acid sequence of SEQ ID NO: 910; a linker comprising the amino acid sequence of (Gly-Gly-Gly-Gly-Ser)n (SEQ ID NO: 1146), wherein n is 4; and a VH comprising the amino acid sequence of SEQ ID NO: 1137.

In some embodiments, the anti-CD3 antibody, or antigen-binding fragment thereof, comprises the six CDRs of antibody ABL-CD3-110 and ABL-CD3-110A. The anti-CD3 antibody, or antigen-binding fragment thereof, may comprise an LCDR1, an LCDR2, and an LCDR3 comprising the amino acid sequences of SEQ ID NOs: 110, 223, and 336, respectively; and an HCDR1, an HCDR2, and an HCDR3 comprising the amino acid sequences of SEQ ID NOs: 454, 567, and 683, respectively. In some embodiments, the anti-CD3 antibody, or antigen-binding fragment thereof, comprises the VL of antibody ABL-CD3-110. The VL may comprise the amino acid sequence of SEQ ID NO: 797. In some embodiments, the anti-CD3 antibody, or antigen-binding fragment thereof, comprises the VH of antibody ABL-CD3-110. The VH may comprise the amino acid sequence of SEQ ID NO: 1024. In some embodiments, the anti-CD3 antibody, or antigen-binding fragment thereof, comprises the VL and VH of antibody ABL-CD3-110. In some embodiments, the anti-CD3 antibody, or antigen-binding fragment thereof, comprises a VL comprising the amino acid sequence of SEQ ID NO: 797 and a VH comprising the amino acid sequence of SEQ ID NO: 1024. In some embodiments, the anti-CD3 antibody, or antigen-binding fragment thereof, comprises the VL of antibody ABL-CD3-110A. The VL may comprise the amino acid sequence of SEQ ID NO: 911. In some embodiments, the anti-CD3 antibody, or antigen-binding fragment thereof, comprises the VH of antibody ABL-CD3-110A. The VH may comprise the amino acid sequence of SEQ ID NO: 1138. In some embodiments, the anti-CD3 antibody, or antigen-binding fragment thereof, comprises the VL and VH of antibody ABL-CD3-110A. In some embodiments, the anti-CD3 antibody, or antigen-binding fragment thereof, comprises a VL comprising the amino acid sequence of SEQ ID NO: 911 and a VH comprising the amino acid sequence of SEQ ID NO: 1138. In some embodiments, the anti-CD3 antibody, or antigen-binding fragment thereof, comprises ABL-CD3-110_scFv_VH-VL. In some embodiments, the anti-CD3 antibody, or antigen-binding fragment thereof, comprises, from N-terminus to C-terminus, a VH comprising the amino acid sequence of SEQ ID NO: 1024; a linker comprising the amino acid sequence of (Gly-Gly-Gly-Gly-Ser)n (SEQ ID NO: 1146), wherein n is 4; and a VL comprising the amino acid sequence of SEQ ID NO: 797. In some embodiments, the anti-CD3 antibody, or antigen-binding fragment thereof, comprises ABL-CD3-110_scFv_VL-VH. In some embodiments, the anti-CD3 antibody, or antigen-binding fragment thereof, comprises, from N-terminus to C-terminus, a VL comprising the amino acid sequence of SEQ ID NO: 797; a linker comprising the amino acid sequence of (Gly-Gly-Gly-Gly-Ser)n (SEQ ID NO: 1146), wherein n is 4; and a VH comprising the amino acid sequence of SEQ ID NO: 1024. In some embodiments, the anti-CD3 antibody, or antigen-binding fragment thereof, comprises ABL-CD3-110A_scFv_VH-VL. In some embodiments, the anti-CD3 antibody, or antigen-binding fragment thereof, comprises, from N-terminus to C-terminus, a VH comprising the amino acid sequence of SEQ ID NO: 1138; a linker comprising the amino acid sequence of (Gly-Gly-Gly-Gly-Ser)n (SEQ ID NO: 1146), wherein n is 4; and a VL comprising the amino acid sequence of SEQ ID NO: 911. In some embodiments, the anti-CD3 antibody, or antigen-binding fragment thereof, comprises ABL-CD3-110A_scFv_VL-VH. In some embodiments, the anti-CD3 antibody, or antigen-binding fragment thereof, comprises, from N-terminus to C-terminus, a VL comprising the amino acid sequence of SEQ ID NO: 911; a linker comprising the amino acid sequence of (Gly-Gly-Gly-Gly-Ser)n (SEQ ID NO: 1146), wherein n is 4; and a VH comprising the amino acid sequence of SEQ ID NO: 1138.

In some embodiments, the anti-CD3 antibody, or antigen-binding fragment thereof, comprises the six CDRs of antibody ABL-CD3-111 and ABL-CD3-111A. The anti-CD3 antibody, or antigen-binding fragment thereof, may comprise an LCDR1, an LCDR2, and an LCDR3 comprising the amino acid sequences of SEQ ID NOs: 111, 224, and 337, respectively; and an HCDR1, an HCDR2, and an HCDR3 comprising the amino acid sequences of SEQ ID NOs: 455, 568, and 684, respectively. In some embodiments, the anti-CD3 antibody, or antigen-binding fragment thereof, comprises the VL of antibody ABL-CD3-111. The VL may comprise the amino acid sequence of SEQ ID NO: 798. In some embodiments, the anti-CD3 antibody, or antigen-binding fragment thereof, comprises the VH of antibody ABL-CD3-111. The VH may comprise the amino acid sequence of SEQ ID NO: 1025. In some embodiments, the anti-CD3 antibody, or antigen-binding fragment thereof, comprises the VL and VH of antibody ABL-CD3-111. In some embodiments, the anti-CD3 antibody, or antigen-binding fragment thereof, comprises a VL comprising the amino acid sequence of SEQ ID NO: 798 and a VH comprising the amino acid sequence of SEQ ID NO: 1025. In some embodiments, the anti-CD3 antibody, or antigen-binding fragment thereof, comprises the VL of antibody ABL-CD3-111A. The VL may comprise the amino acid sequence of SEQ ID NO: 912. In some embodiments, the anti-CD3 antibody, or antigen-binding fragment thereof, comprises the VH of antibody ABL-CD3-111A. The VH may comprise the amino acid sequence of SEQ ID NO: 1139. In some embodiments, the anti-CD3 antibody, or antigen-binding fragment thereof, comprises the VL and VH of antibody ABL-CD3-111A. In some embodiments, the anti-CD3 antibody, or antigen-binding fragment thereof, comprises a VL comprising the amino acid sequence of SEQ ID NO: 912 and a VH comprising the amino acid sequence of SEQ ID NO: 1139. In some embodiments, the anti-CD3 antibody, or antigen-binding fragment thereof, comprises ABL-CD3-111_scFv_VH-VL. In some embodiments, the anti-CD3 antibody, or antigen-binding fragment thereof, comprises, from N-terminus to C-terminus, a VH comprising the amino acid sequence of SEQ ID NO: 1025; a linker comprising the amino acid sequence of (Gly-Gly-Gly-Gly-Ser)n (SEQ ID NO: 1146), wherein n is 4; and a VL comprising the amino acid sequence of SEQ ID NO: 798. In some embodiments, the anti-CD3 antibody, or antigen-binding fragment thereof, comprises ABL-CD3-111_scFv_VL-VH. In some embodiments, the anti-CD3 antibody, or antigen-binding fragment thereof, comprises, from N-terminus to C-terminus, a VL comprising the amino acid sequence of SEQ ID NO: 798; a linker comprising the amino acid sequence of (Gly-Gly-Gly-Gly-Ser)n (SEQ ID NO: 1146), wherein n is 4; and a VH comprising the amino acid sequence of SEQ ID NO: 1025. In some embodiments, the anti-CD3 antibody, or antigen-binding fragment thereof, comprises ABL-CD3-111A_scFv_VH-VL. In some embodiments, the anti-CD3 antibody, or antigen-binding fragment thereof, comprises, from N-terminus to C-terminus, a VH comprising the amino acid sequence of SEQ ID NO: 1139; a linker comprising the amino acid sequence of (Gly-Gly-Gly-Gly-Ser)n (SEQ ID NO: 1146), wherein n is 4; and a VL comprising the amino acid sequence of SEQ ID NO: 912. In some embodiments, the anti-CD3 antibody, or antigen-binding fragment thereof, comprises ABL-CD3-111A_scFv_VL-VH. In some embodiments, the anti-CD3 antibody, or antigen-binding fragment thereof, comprises, from N-terminus to C-terminus, a VL comprising the amino acid sequence of SEQ ID NO: 912; a linker comprising the amino acid sequence of (Gly-Gly-Gly-Gly-Ser)n (SEQ ID NO: 1146), wherein n is 4; and a VH comprising the amino acid sequence of SEQ ID NO: 1139.

In some embodiments, the anti-CD3 antibody, or antigen-binding fragment thereof, comprises the six CDRs of antibody ABL-CD3-112 and ABL-CD3-112A. The anti-CD3 antibody, or antigen-binding fragment thereof, may comprise an LCDR1, an LCDR2, and an LCDR3 comprising the amino acid sequences of SEQ ID NOs: 112, 225, and 338, respectively; and an HCDR1, an HCDR2, and an HCDR3 comprising the amino acid sequences of SEQ ID NOs: 456, 569, and 685, respectively. In some embodiments, the anti-CD3 antibody, or antigen-binding fragment thereof, comprises the VL of antibody ABL-CD3-112. The VL may comprise the amino acid sequence of SEQ ID NO: 799. In some embodiments, the anti-CD3 antibody, or antigen-binding fragment thereof, comprises the VH of antibody ABL-CD3-112. The VH may comprise the amino acid sequence of SEQ ID NO: 1026. In some embodiments, the anti-CD3 antibody, or antigen-binding fragment thereof, comprises the VL and VH of antibody ABL-CD3-112. In some embodiments, the anti-CD3 antibody, or antigen-binding fragment thereof, comprises a VL comprising the amino acid sequence of SEQ ID NO: 799 and a VH comprising the amino acid sequence of SEQ ID NO: 1026. In some embodiments, the anti-CD3 antibody, or antigen-binding fragment thereof, comprises the VL of antibody ABL-CD3-112A. The VL may comprise the amino acid sequence of SEQ ID NO: 913. In some embodiments, the anti-CD3 antibody, or antigen-binding fragment thereof, comprises the VH of antibody ABL-CD3-112A. The VH may comprise the amino acid sequence of SEQ ID NO: 1140. In some embodiments, the anti-CD3 antibody, or antigen-binding fragment thereof, comprises the VL and VH of antibody ABL-CD3-112A. In some embodiments, the anti-CD3 antibody, or antigen-binding fragment thereof, comprises a VL comprising the amino acid sequence of SEQ ID NO: 913 and a VH comprising the amino acid sequence of SEQ ID NO: 1140. In some embodiments, the anti-CD3 antibody, or antigen-binding fragment thereof, comprises ABL-CD3-112_scFv_VH-VL. In some embodiments, the anti-CD3 antibody, or antigen-binding fragment thereof, comprises, from N-terminus to C-terminus, a VH comprising the amino acid sequence of SEQ ID NO: 1026; a linker comprising the amino acid sequence of (Gly-Gly-Gly-Gly-Ser)n (SEQ ID NO: 1146), wherein n is 4; and a VL comprising the amino acid sequence of SEQ ID NO: 799. In some embodiments, the anti-CD3 antibody, or antigen-binding fragment thereof, comprises ABL-CD3-112_scFv_VL-VH. In some embodiments, the anti-CD3 antibody, or antigen-binding fragment thereof, comprises, from N-terminus to C-terminus, a VL comprising the amino acid sequence of SEQ ID NO: 799; a linker comprising the amino acid sequence of (Gly-Gly-Gly-Gly-Ser)n (SEQ ID NO: 1146), wherein n is 4; and a VH comprising the amino acid sequence of SEQ ID NO: 1026. In some embodiments, the anti-CD3 antibody, or antigen-binding fragment thereof, comprises ABL-CD3-112A_scFv_VH-VL. In some embodiments, the anti-CD3 antibody, or antigen-binding fragment thereof, comprises, from N-terminus to C-terminus, a VH comprising the amino acid sequence of SEQ ID NO: 1140; a linker comprising the amino acid sequence of (Gly-Gly-Gly-Gly-Ser)n (SEQ ID NO: 1146), wherein n is 4; and a VL comprising the amino acid sequence of SEQ ID NO: 913. In some embodiments, the anti-CD3 antibody, or antigen-binding fragment thereof, comprises ABL-CD3-112A_scFv_VL-VH. In some embodiments, the anti-CD3 antibody, or antigen-binding fragment thereof, comprises, from N-terminus to C-terminus, a VL comprising the amino acid sequence of SEQ ID NO: 913; a linker comprising the amino acid sequence of (Gly-Gly-Gly-Gly-Ser)n (SEQ ID NO: 1146), wherein n is 4; and a VH comprising the amino acid sequence of SEQ ID NO: 1140.

In some embodiments the anti-CD3 antibody, or antigen-binding fragment thereof, comprises the six CDRs of antibody ABL-CD3-113 and ABL-CD3-113A. The anti-CD3 antibody, or antigen-binding fragment thereof, may comprise an LCDR1, an LCDR2, and an LCDR3 comprising the amino acid sequences of SEQ ID NOs: 113, 226, and 339, respectively; and an HCDR1, an HCDR2, and an HCDR3 comprising the amino acid sequences of SEQ ID NOs: 457, 570, and 686, respectively. In some embodiments, the anti-CD3 antibody, or antigen-binding fragment thereof, comprises the VL of antibody ABL-CD3-113. The VL may comprise the amino acid sequence of SEQ ID NO: 800. In some embodiments, the anti-CD3 antibody, or antigen-binding fragment thereof, comprises the VH of antibody ABL-CD3-113. The VH may comprise the amino acid sequence of SEQ ID NO: 1027. In some embodiments, the anti-CD3 antibody, or antigen-binding fragment thereof, comprises the VL and VH of antibody ABL-CD3-113. In some embodiments, the anti-CD3 antibody, or antigen-binding fragment thereof, comprises a VL comprising the amino acid sequence of SEQ ID NO: 800 and a VH comprising the amino acid sequence of SEQ ID NO: 1027. In some embodiments, the anti-CD3 antibody, or antigen-binding fragment thereof, comprises the VL of antibody ABL-CD3-113A. The VL may comprise the amino acid sequence of SEQ ID NO: 914. In some embodiments, the anti-CD3 antibody, or antigen-binding fragment thereof, comprises the VH of antibody ABL-CD3-113A. The VH may comprise the amino acid sequence of SEQ ID NO: 1141. In some embodiments, the anti-CD3 antibody, or antigen-binding fragment thereof, comprises the VL and VH of antibody ABL-CD3-113A. In some embodiments, the anti-CD3 antibody, or antigen-binding fragment thereof, comprises a VL comprising the amino acid sequence of SEQ ID NO: 914 and a VH comprising the amino acid sequence of SEQ ID NO: 1141. In some embodiments, the anti-CD3 antibody, or antigen-binding fragment thereof, comprises ABL-CD3-113_scFv_VH-VL. In some embodiments, the anti-CD3 antibody, or antigen-binding fragment thereof, comprises, from N-terminus to C-terminus, a VH comprising the amino acid sequence of SEQ ID NO: 1027; a linker comprising the amino acid sequence of (Gly-Gly-Gly-Gly-Ser)n (SEQ ID NO: 1146), wherein n is 4; and a VL comprising the amino acid sequence of SEQ ID NO: 800. In some embodiments, the anti-CD3 antibody, or antigen-binding fragment thereof, comprises ABL-CD3-113_scFv_VL-VH. In some embodiments, the anti-CD3 antibody, or antigen-binding fragment thereof, comprises, from N-terminus to C-terminus, a VL comprising the amino acid sequence of SEQ ID NO: 800; a linker comprising the amino acid sequence of (Gly-Gly-Gly-Gly-Ser)n (SEQ ID NO: 1146), wherein n is 4; and a VH comprising the amino acid sequence of SEQ ID NO: 1027. In some embodiments, the anti-CD3 antibody, or antigen-binding fragment thereof, comprises ABL-CD3-113A_scFv_VH-VL. In some embodiments, the anti-CD3 antibody, or antigen-binding fragment thereof, comprises, from N-terminus to C-terminus, a VH comprising the amino acid sequence of SEQ ID NO: 1141; a linker comprising the amino acid sequence of (Gly-Gly-Gly-Gly-Ser)n (SEQ ID NO: 1146), wherein n is 4; and a VL comprising the amino acid sequence of SEQ ID NO: 914. In some embodiments, the anti-CD3 antibody, or antigen-binding fragment thereof, comprises ABL-CD3-113A_scFv_VL-VH. In some embodiments, the anti-CD3 antibody, or antigen-binding fragment thereof, comprises, from N-terminus to C-terminus, a VL comprising the amino acid sequence of SEQ ID NO: 914; a linker comprising the amino acid sequence of (Gly-Gly-Gly-Gly-Ser)n (SEQ ID NO: 1146), wherein n is 4; and a VH comprising the amino acid sequence of SEQ ID NO: 1141.

In some embodiments, the VL comprises an amino acid sequence of one of SEQ ID NOs: 688-914. In some embodiments, the VH comprises an amino acid sequence of one of SEQ ID NOs: 915-1141. In some embodiments, the VL comprises an amino acid sequence of one of SEQ ID NOs: 688-914, and the VH comprises an amino acid sequence of one of SEQ ID NOs: 915-1141. In some embodiments, the VH and the VL are from the same anti-CD3 AlivaMab® Antibody.

In some embodiments, an anti-CD3 antibody, or an antigen-binding fragment thereof, of the disclosure is human. In some embodiments, the human anti-CD3 antibody, or an antigen-binding fragment thereof, comprises a human IgH chain and a human Igκ chain. In some embodiments, the human anti-CD3 antibody, or an antigen-binding fragment thereof, comprises a human IgH chain and a human Igλ chain. In some embodiments, the isotype of the anti-CD3 antibody is selected from IgM, IgD, IgG (such as IgG1, IgG2, IgG3, and IgG4), IgA and IgE. In some embodiments, the isotype of the anti-CD3 antibody is IgG. In some embodiments, the isotype of the anti-CD3 antibody is selected from IgG1, IgG2, IgG3, and IgG4.

In some embodiments, the anti-CD3 antibody binds an Fc receptor (FcR) selected from an FcγR, an FcεR, and an FcαR. In some embodiments, the anti-CD3 antibody binds an FcγR selected from FcγRI (CD64), FcγRII (CD32), and FcγRIII (CD16), including isoforms thereof. In some embodiments, the Fc domain of the anti-CD3 antibody comprises a mutation so that it preferentially binds a particular FcγR (see, e.g., U.S. Pat. No. 6,737,056 and U.S. 2015/0031862).

In some embodiments, the anti-CD3 antibody has reduced effector function. The anti-CD3 antibody may exhibit reduced binding to an Fc receptor (FcR) selected from an FcγR, an FcεR, and an FcαR. In some embodiments, the anti-CD3 antibody exhibits reduced binding to an FcγR selected from FcγRI (CD64), FcγRII (CD32), and FcγRIII (CD16), including isoforms thereof. In some embodiments, the Fc domain of the anti-CD3 antibody comprises a mutation so that reduces binding a particular FcγR.

The anti-CD3 antibody may have no effector function. In some embodiments, the anti-CD3 antibody does not bind an Fc receptor (FcR) selected from an FcγR, an FcεR, and an FcαR. In some embodiments, the anti-CD3 antibody does not bind an FcγR selected from FcγRI (CD64), FcγRII (CD32), and FcγRIII (CD16), including isoforms thereof. In some embodiments, the Fc domain of the anti-CD3 antibody comprises a mutation so that it does not bind a particular FcγR. The engineering of antibodies to reduce or eliminate effector function is well-known in the art (see, e.g., Stewart et at (2014) JITC 2:29, Dumet C. et al. MAbs, 2019, vol. 11(8): 1341-1350; and Desjarlais, J R. and Lazar, GA, Exp. Cell. Res., 2011, vol. 317(9): 1278-1285; see also WO 89/07142; WO 94/28027; WO 94/29351; WO 95/26403; WO 2000/42072; WO 2004/029207; WO 2005/018572; WO 2005/007809; WO 2011/066501; WO 2011/149999; WO 2012/130831; WO 2014/121087; WO 2017/079369; WO 2018/071919; U.S. Pat. Nos. 8,961,967; 10,259,887; 10,501,543; US 2006/0134105).

In some embodiments, the Fc domain of an antibody disclosed herein comprises one or more amino acid modifications that reduces or eliminates one or more effector functions. In some embodiments, the modified Fc domain is derived from an IgG1 Fc domain. Optionally, the modified Fc domain is derived from an IgG1 constant region. The modified Fc domain may be derived from an IgG2 Fc domain. In some embodiments, the modified Fc domain is derived from an IgG2 constant region. The modified Fc domain may be derived from an IgG4 Fc domain region. In some embodiments, the modified Fc domain is derived from an IgG4 constant region. In some embodiments, the modified Fc domain is derived from a combination of Fc domains from more than one isotype of constant region.

In some embodiments, the antibody comprises a modified Fc domain derived from an IgG1 molecule, wherein the modified Fc domain comprises an amino acid modification at any one of the positions selected from the group consisting of E216, R217, K218, C219, C220, C226, C229, P230, E233, L234, L235, G236, G237, P238, S239, V240, F241, K246, L251, T260, D265, V266, H268, W277, N297, E318, K322, P329, A330, P331, Q347, N348, T350, L351, K360, T366, N390, K392, T394, D399, S400, F405, Y407, K409, T411 or a combination such amino acid modifications, wherein the numbering of amino acid residues is according to the EU index as set forth in Edelman G M et al., Proc. Natl. Acad. USA, 63, 78-85 (1969). In preferred embodiments, the modified Fc domain derived from IgG1 comprises an amino acid modification selected from the group consisting of C220S, C226S, C229S, P230S, E233P, L234A, L234F, L234V, L235A, L235E, L235V, G236E, G237A, P238S, D265S, D265A, H268Q, W277T, N297G, N297Q, N297D, N297A, E318A, K322A, P329G, P329A, A330S, P331S, Q347R, Q347E, Q347K, T350V, L351Y, K360D, K360E, T366A, T366I, T366L, T366M, T366V, N390R, N390K, N390D, K392V, K392M, K392R, K392L, K392F, K392E, T394W, D399R, D399W, D399K, S400E, S400D, S400R, S400K, F405A, F405I, F405M, F405T, F405S, F405V, F405W, Y407A, Y407I, Y407L, Y407V, K409F, K409I, K409S, K409W, T411N, T411R, T411Q, T411K, T411D, T411E, T411W, ΔE216-E222, K246R/L251E/T260R, InR234/235, InV235/236, InR236/237, InR237/238, InV238/239, InN238/239, InL238/239, InE238/239, InG238/239, InS239/240, InG240/241, InE240/241, InG240/241, InL238/239/P238Q, InE238/239/N348A, InS239/240/V266A, and InR237/238/G236A or a combination thereof.

In some embodiments, the antibody comprises a modified Fc domain derived from an IgG2 molecule, wherein the modified Fc domain comprises an amino acid modification at any one of the positions selected from the group consisting of V234, G237, P238, H268, V309, A330, and P331 or a combination thereof, wherein the numbering of amino acid residues is according to the EU numbering as set forth in Edelman. In preferred embodiments, modified Fc domain derived from IgG2 comprises an amino acid modification selected from the group consisting of V234A, G237A, P238S, H268Q, H268A, V309L, A330S, P331S, or a combination thereof.

In some embodiments, the antibody comprises a modified Fc domain derived from an IgG4 molecule, wherein the modified Fc domain comprises an amino acid modification at any one of the positions selected from the group consisting of S228, L235, L236, G237, E318, and N297 or a combination thereof, wherein the numbering of amino acid residues is according to the EU index as set forth in Edelman G M et al., Proc. Natl. Acad. USA, 63, 78-85 (1969). In preferred embodiments, the modified Fc domain derived from IgG4 comprises an amino acid modification selected from the group consisting of S228P, L235A, L235E, L236E, G237A, E318A, and N297Q or a combination thereof.

### Bispecific Antibodies

One aspect of the present disclosure provides a bispecific antibody. In some embodiments, the bispecific antibody comprises an anti-CD3 antibody, or antigen-binding fragment thereof, disclosed herein. In some embodiments, the bispecific antibody comprises an anti-CD3 antibody, or antigen-binding fragment thereof, disclosed herein and a targeting antibody, or an antigen-binding fragment thereof. In some embodiments, the targeting antibody, or antigen-binding fragment thereof, is an anti-tumor antibody, or an antigen-binding fragment thereof.

In some embodiments, the anti-CD3 antibody, or antigen-binding fragment thereof, comprises a single-chain Fv (scFv). In some embodiments, the anti-CD3 antibody, or antigen-binding fragment thereof, comprises a single-chain Fv (scFv) operably linked to a first Fc domain. In some embodiments, the anti-CD3 antibody, or antigen-binding fragment thereof, comprises a Fab fragment. In some embodiments, the anti-CD3 antibody, or antigen-binding fragment thereof, comprises a Fab fragment operably linked to a first Fc domain. In some embodiments, the anti-CD3 antibody, or antigen-binding fragment thereof, comprises a Fab′. In some embodiments, the anti-CD3 antibody, or antigen-binding fragment thereof, comprises a Fab′ operably linked to a first Fc domain. In some embodiments, the anti-CD3 antibody, or antigen-binding fragment thereof, comprises a F(ab′)₂. In some embodiments, the anti-CD3 antibody, or antigen-binding fragment thereof, comprises a F(ab′)₂ operably linked to a first Fc domain. In some embodiments, the anti-CD3 antibody, or antigen-binding fragment thereof, comprises an Fv. In some embodiments, the anti-CD3 antibody, or antigen-binding fragment thereof, comprises an Fv operably linked to a first Fc domain. In some embodiments, the anti-CD3 antibody, or antigen-binding fragment thereof, comprises a complete heavy chain, comprising a first Fc domain. As used herein, a complete heavy chain includes heavy chains that do not contain all of their amino acid residues as a result of C-terminal clipping. In some embodiments, the anti-CD3 antibody, or antigen-binding fragment thereof, comprises a complete light chain. In some embodiments, the anti-CD3 antibody, or antigen-binding fragment thereof, comprises a complete heavy chain, comprising a first Fc domain, and a complete light chain.

In some embodiments, the anti-CD3 antibody, or antigen-binding fragment thereof, is operably linked to the first Fc domain via a first linker. In some embodiments, the anti-CD3 antibody, or antigen-binding fragment thereof, is operably linked to the N-terminus of the first Fc domain. In some embodiments, the anti-CD3 antibody, or antigen-binding fragment thereof, is operably linked to the C-terminus of the first Fc domain. In some embodiments, the first linker is an antibody hinge region. In some embodiments, the first linker is a variable length Gly-Ser linker. In some embodiments, the first linker comprises a linker selected from the group consisting of (Gly-Gly-Gly-Gly-Ser)n (SEQ ID NO: 1238), (Ser-Ser-Ser-Ser-Gly)n (SEQ ID NO: 1147), (Gly-Ser-Ser-Gly-Gly)n (SEQ ID NO: 1148), and (Gly-Gly-Ser-Gly-Gly)n (SEQ ID NO: 1149), where n is an integer between 1 and 5. In some embodiments, the first linker comprises the amino acid sequence of (Gly-Gly-Gly-Gly-Ser)n (SEQ ID NO: 1237), wherein n is 1. In some embodiments, the linker comprises the amino acid sequence of (Gly-Gly-Gly-Gly-Ser)n (SEQ ID NO: 1146), wherein n is 4.

In some embodiments, the targeting antibody in the anti-CD3 bispecific antibody is an anti-tumor antibody, or an antigen-binding fragment thereof. In some embodiments, the anti-tumor antibody, or antigen-binding fragment thereof, comprises a single-chain Fv (scFv). In some embodiments, the anti-tumor antibody, or antigen-binding fragment thereof, comprises a single-chain Fv (scFv) operably linked to a second Fc domain. In some embodiments, the anti-tumor antibody, or antigen-binding fragment thereof, comprises a Fab fragment. In some embodiments, the anti-tumor antibody, or antigen-binding fragment thereof, comprises a Fab fragment operably linked to a second Fc domain. In some embodiments, the anti-tumor antibody, or antigen-binding fragment thereof, comprises a Fab′. In some embodiments, the anti-tumor antibody, or antigen-binding fragment thereof, comprises a Fab′ operably linked to a second Fc domain. In some embodiments, the anti-tumor antibody, or antigen-binding fragment thereof, comprises a F(ab′)₂. In some embodiments, the anti-tumor antibody, or antigen-binding fragment thereof, comprises a F(ab′)₂ operably linked to a second Fc domain. In some embodiments, the anti-tumor antibody, or antigen-binding fragment thereof, comprises an Fv. In some embodiments, the anti-tumor antibody, or antigen-binding fragment thereof, comprises an Fv operably linked to a second Fc domain. In some embodiments, the anti-tumor antibody, or antigen-binding fragment thereof, comprises a complete heavy chain, comprising a second Fc domain. As used herein, a complete heavy chain includes heavy chains that do not contain all of their amino acid residues as a result of C-terminal clipping. In some embodiments, the anti-tumor antibody, or antigen-binding fragment thereof, comprises a complete light chain. In some embodiments, the anti-tumor antibody, or antigen-binding fragment thereof, comprises a complete heavy chain, comprising a second Fc domain, and a complete light chain.

In some embodiments, the anti-tumor antibody, or antigen-binding fragment thereof, is operably linked to the second Fc domain via a second linker. In some embodiments, the anti-tumor antibody, or antigen-binding fragment thereof, is operably linked to the N-terminus of the second Fc domain. In some embodiments, the anti-tumor antibody, or antigen-binding fragment thereof, is operably linked to the C-terminus of the second Fc domain. In some embodiments, the second linker is an antibody hinge region. In some embodiments, the second linker is a variable length Gly-Ser linker. In some embodiments, the second linker comprises a linker selected from the group consisting of (Gly-Gly-Gly-Gly-Ser)n (SEQ ID NO: 1238), (Ser-Ser-Ser-Ser-Gly)n (SEQ ID NO: 1147), (Gly-Ser-Ser-Gly-Gly)n (SEQ ID NO: 1148), and (Gly-Gly-Ser-Gly-Gly)n (SEQ ID NO: 1149), where n is an integer between 1 and 5. In some embodiments, the second linker comprises the amino acid sequence of (Gly-Gly-Gly-Gly-Ser)n (SEQ ID NO: 1237), wherein n is 1. In some embodiments, the second linker comprises the amino acid sequence of (Gly-Gly-Gly-Gly-Ser)n (SEQ ID NO: 1146), wherein n is 4.

It is known in the art the mutations can be introduced into the Fc domains of bispecific antibodies to promote heterodimerization over homodimerization. See, e.g., Ha, J H et al., “Immunoglobulin Fc heterodimer platform technology: from design to applications in therapeutic antibodies and proteins,” Front. Immunol. 2016, vol. 7: 394, incorporated by reference herein in its entirety. Such mutations include “knobs-into-holes” (KIH) mutations; HA-TF (and related) mutations; skew mutations; ZW mutations; 7.8.60 (and related) mutations; DD-KK (and related) mutations; EW-RVT mutations; EW-RVTs-s mutations; SEED mutations, A107 mutations, Duobody mutations and Crossmab mutations. In some embodiments, the first Fc domain is derived from an IgG1 constant region. In some embodiments, the second Fc domain is derived from an IgG1 constant region. In some embodiments, the first and second Fc domains are derived from IgG1 constant regions.

In some embodiments, the first and second Fc domains comprise KIH mutations. See, e.g., Ridgway J B et al., Protein Eng (1996) 9(7):617-21; Atwell S et al., J Mol Biol (1997) 270(1):26-35; and Merchant et al., Nat Biotechnol, 1998, 16:677-81, each of which is incorporated by reference herein. Non-limiting examples of “knob” mutations include S354C and T366W. Non-limiting examples of “hole” mutations include Y349C, T366S, L368A, and Y407V. The “hole” may be further mutated to eliminate protein A binding (H435R/Y436F). In this way, the knob-containing Fc domain can bind to protein A, and the hole-containing Fc can bind to Fab-selective resins, offering multiple purification options.

In some embodiments, the first Fc domain comprises a T366W mutation and the second Fc domain comprises T366S/L368A/Y407V mutations. In some embodiments, the first Fc domain comprises T366S/L368A/Y407V mutations and the second Fc domain comprises a T366W mutation. In some embodiments, the first and second Fc domains comprise KIHs-s mutations. In some embodiments, the first Fc domain comprises S354C/T366W mutations and the second Fc domain comprises Y349C/T366S/L368A/Y407V mutations. In some embodiments, the first Fc domain comprises Y349C/T366S/L368A/Y407V mutations and the second Fc domain comprises S354C/T366W mutation. In some embodiments, the first Fc domain comprises S354C/T366W mutations and the second Fc domain comprises Y349C/T366S/L368A/Y407V/H435R/Y436F mutations. In some embodiments, the first Fc domain comprises Y349C/T366S/L368A/Y407V/H435R/Y436F mutations and the second Fc domain comprises S354C/T366W mutation. In some embodiments, the first Fc domain comprises S354C/T366W/H435R/Y436F mutations and the second Fc domain comprises Y349C/T366S/L368A/Y407V mutations. In some embodiments, the first Fc domain comprises Y349C/T366S/L368A/Y407V mutations and the second Fc domain comprises S354C/T366W/H435R/Y436F mutations. Each of the foregoing mutations is according to EU numbering.

In some embodiments, the knob-containing Fc domain comprises the amino acid sequence DKTHTCPPCPAPEFEGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPASIEK TISKAKGQPREPQVYTLPPCREEMTKNQVSLWCLVKGFYPSDIAVEWESNGQPENNY KTTPPVLDSDGSFFLYSKLTVDKSRWQQGNVFSCSVMHEALHNHYTQKSLSLSPGK (SEQ ID NO: 1142). In some embodiments, the hole-containing Fc domain comprises the amino sequence DKTHTCPPCPAPEFEGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPASIEK TISKAKGQPREPQVCTLPPSREEMTKNQVSLSCAVKGFYPSDIAVEWESNGQPENNY KTTPPVLDSDGSFFLVSKLTVDKSRWQQGNVFSCSVMHEALHNRFTQKSLSLSPGK (SEQ ID NO: 1143). In some embodiments, the first Fc domain comprises the amino acid sequence of SEQ ID NO: 1142. In some embodiments, the first Fc domain comprises the amino acid sequence of SEQ ID NO: 1143. In some embodiments, the second Fc domain comprises the amino acid sequence of SEQ ID NO: 1142. In some embodiments, the second Fc domain comprises the amino acid sequence of SEQ ID NO: 1143. In some embodiments, the first and second Fe domains comprise the amino acid sequences of SEQ ID NOs: 1142 and 1143, respectively. In some embodiments, the first and second Fe domains comprise the amino acid sequences of SEQ ID NOs: 1143 and 1142, respectively.

In some embodiments, the knob-containing constant region comprises the amino acid sequence of ASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKRVEPKSC DKTHTCPPCPAPEFEGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALPASIEK TISKAKGQPREPQVYTLPPCREEMTKNQVSLWCLVKGFYPSDIAVEWESNGQPENNY KTTPPVLDSDGSFFLYSKLTVDKSRWQQGNVFSCSVMHEALHNHYTQKSLSLSPGK (SEQ ID NO: 1144). In some embodiments, the hole-containing constant region comprises the amino acid sequence of ASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKRVEPKSCDKTHTCPPCPAPEF EGGPSVFLFPPKPKDTLMISRTPEVTCVVVDVSSHEDPEVKFNWYVDGVEVHNAKTKP REEQYNSTYRVVSVLTVLHQDWLNGKEYKCKVSNKALPASIEKTISKAKGQPREPQV CTLPPSREEMTKNQVSLSCAVKGFYPSDIAVEWESNGQPENNYKTTPPVLDSDGSFFL VSKLTVDKSRWQQGNVFSCSVMHEALHNRFTQKSLSLSPGK (SEQ ID NO: 1145). In some embodiments, the anti-CD3 antibody comprises a constant region comprising the amino acid sequence of SEQ ID NO: 1144. In some embodiments, the anti-CD3 antibody comprises a constant region comprising the amino acid sequence of SEQ ID NO: 1145. In some embodiments, the anti-tumor antibody comprises a constant region comprising the amino acid sequence of SEQ ID NO: 1144. In some embodiments, the anti-tumor antibody comprises a constant region comprising the amino acid sequence of SEQ ID NO: 1145. In some embodiments, the anti-CD3 antibody comprises a constant region comprising the amino acid sequence of SEQ ID NO: 1144, and the anti-tumor antibody comprises a constant region comprising the amino acid sequence of SEQ ID NO: 1145. In some embodiments, the anti-CD3 antibody comprises a constant region comprising the amino acid sequence of SEQ ID NO: 1145, and the anti-tumor antibody comprises a constant region comprising the amino acid sequence of SEQ ID NO: 1144.

In some embodiments, the anti-CD3 antibody, or antigen-binding fragment thereof, is operably linked to a constant region comprising the amino acid sequence of SEQ ID NO: 1144. In some embodiments, the anti-CD3 antibody, or antigen-binding fragment thereof, is operably linked to a constant region comprising the amino acid sequence of SEQ ID NO: 1145. In some embodiments, the anti-tumor antibody, or antigen-binding fragment thereof, is operably linked to a constant region comprising the amino acid sequence of SEQ ID NO: 1144. In some embodiments, the anti-tumor antibody, or antigen-binding fragment thereof, is operably linked to a constant region comprising the amino acid sequence of SEQ ID NO: 1145. In some embodiments, the anti-CD3 antibody, or antigen-binding fragment thereof, is operably linked to a constant region comprising the amino acid sequence of SEQ ID NO: 1144, and the anti-tumor antibody, or antigen-binding fragment thereof, is operably linked to a constant region comprising the amino acid sequence of SEQ ID NO: 1145. In some embodiments, the anti-CD3 antibody, or antigen-binding fragment thereof, is operably linked to a constant region comprising the amino acid sequence of SEQ ID NO: 1145, and the anti-tumor antibody, or antigen-binding fragment thereof, is operably linked to a constant region comprising the amino acid sequence of SEQ ID NO: 1144.

In some embodiments, the first and second Fc domains comprise HA-TF and related mutations. See, e,g., Moore G L, et al., MAbs (2011) 3(6):546-57, incorporated herein by reference. In some embodiments, the first Fc domain comprises a S364H mutation, and the second Fc domain comprises a Y349T mutation. In some embodiments, the first Fc domain comprises a Y349T mutation, and the second Fc domain comprises a S364H mutation. In some embodiments, the first Fc domain comprises a F405A mutation, and the second Fc domain comprises a T394F mutation. In some embodiments, the first Fc domain comprises a T394F mutation, and the second Fc domain comprises a F405A mutation. In some embodiments, the first Fc domain comprises S364H/T394F mutations, and the second Fc domain comprises Y349T/F405A mutations. In some embodiments, the first Fc domain comprises Y349T/F405A mutations, and the second Fc domain comprises S364H/T394F mutations. In some embodiments, the first Fc domain comprises S364H/F405A mutations and the second Fc domain Y349T/T394F mutations. In some embodiments, the first Fc domain comprises Y349T/T394F mutations and the second Fc domain S364H/F405A mutations. Each of the foregoing mutations is according to EU numbering.

In some embodiments, the first and second Fc domains comprise skew mutations. See, e.g., U.S. Pat. No. 10,793,632, incorporated herein by reference. In some embodiments, the first Fc domain comprises S364K/E357Q mutations, and the second Fc domain comprises L368D/K370S mutations. In some embodiments, the first Fc domain comprises L368D/K370S mutations, and the second Fc domain comprises S364K/E357Q mutations. In some embodiments, the first Fc domain comprises L368D/K370S mutations, and the second Fc domain comprises a S364K mutation. In some embodiments, the first Fc domain comprises a S364K mutation, and the second Fc domain comprises L368D/K370S mutations. In some embodiments, the first Fc domain comprises L368E/K370S mutations, and the second Fc domain comprises a S364K mutation. In some embodiments, the first Fc domain comprises a S364K mutation, and the second Fc domain comprises L368E/K370S mutations. In some embodiments, the first Fc domain comprises T411E/K360E/Q362E mutations, and the second Fc domain comprises a D401K mutation. In some embodiments, the first Fc domain comprises a D401K mutation, and the second Fc domain comprises T411E/K360E/Q362E mutations. In some embodiments, the first Fc domain comprises L368D/K370S mutations, and the second Fc domain comprises S364K/E357L mutations. In some embodiments, the first Fc domain comprises S364K/E357L mutations, and the second Fc domain comprises L368D/K370S mutations. In some embodiments, the first Fc domain comprises a K370S mutation, and the second Fc domain comprises S364K/E357Q mutations. In some embodiments, the first Fc domain comprises S364K/E357Q mutations, and the second Fc domain comprises a K370S mutation. In some embodiments, the first Fc domain comprises T366S/L368A/Y407V mutations, and the second Fc domain comprises a T366W mutation. In some embodiments, the first Fc domain comprises a T366W mutation, and the second Fc domain comprises T366S/L368A/Y407V mutations. In some embodiments, the first Fc domain comprises T366S/L368A/Y407V/Y349C mutations, and the second Fc domain comprises T366W/S354C mutations. In some embodiments, the first Fc domain comprises T366W/S354C mutations, and the second Fc domain comprises T366S/L368A/Y407V/Y349C mutations. Each of the foregoing mutations is according to EU numbering.

In some embodiments, the first and second Fe domains comprise ZW mutations. See, e.g., Von Kreudenstein T S, et al., MAbs (2013) 5(5):646-54, incorporated herein by reference. In some embodiments, the first Fc domain comprises F405A/Y407V mutations, and the second Fc domain comprises a T394W mutation. In some embodiments, the first Fc domain comprises a T394W mutation, and the second Fc domain comprises F405A/Y407V mutations. In some embodiments, the first Fc domain comprises F405A/Y407V mutations, and the second Fc domain comprises T366I/T394W mutations. In some embodiments, the first Fc domain comprises T366I/T394W mutations, and the second Fc domain comprises F405A/Y407V mutations. In some embodiments, the first Fc domain comprises F405A/Y407V mutations, and the second Fc domain comprises T366L/T394W mutations. In some embodiments, the first Fc domain comprises T366L/T394W mutations, and the second Fc domain comprises F405A/Y407V mutations. In some embodiments, the first Fc domain comprises F405A/Y407V mutations, and the second Fc domain comprises T366L/K392M/T394W mutations. In some embodiments, the first Fc domain comprises T366L/K392M/T394W mutations, and the second Fc domain comprises F405A/Y407V mutations. In some embodiments, the first Fc domain comprises L351Y/F405A/Y407V mutations, and the second Fc domain comprises T366L/K329M/T394W mutations. In some embodiments, the first Fc domain comprises T366L/K329M/T394W mutations, and the second Fc domain comprises L351Y/F405A/Y407V mutations. In some embodiments, the first Fc domain comprises T350V/L351Y/F405A/Y407V mutations, and the second Fc domain comprises T350V/T366L/K392M/T394W mutations. In some embodiments, the first Fc domain comprises T350V/T366L/K392M/T394W mutations, and the second Fc domain comprises T350V/L351Y/F405A/Y407V mutations. In some embodiments, the first Fc domain comprises T350V/L351Y/F405A/Y407V mutations, and the second Fc domain comprises T350V/T366L/K392L/T394W mutations. In some embodiments, the first Fc domain comprises T350V/T366L/K392L/T394W mutations, and the second Fc domain comprises T350V/L351Y/F405A/Y407V mutations. Each of the foregoing mutations is according to EU numbering.

In some embodiments, the first and second Fe domains comprise 7.8.60 mutations or related mutations. See, e.g., Leaver-Fay A, et al., Structure (2016) 24(4):641-51, incorporated herein by reference. In some embodiments, the first Fc domain comprises D399M/Y407A mutations, and the second Fc domain comprises T366V/K409V mutations. In some embodiments, the first Fc domain comprises T366V/K409V mutations, and the second Fc domain comprises D399M/Y407A mutations. In some embodiments, the first Fc domain comprises K360D/D399M/Y407A mutations, and the second Fc domain comprises E345R/Q347R/T366V/K409V mutations. In some embodiments, the first Fc domain comprises E345R/Q347R/T366V/K409V mutations, and the second Fc domain comprises K360D/D399M/Y407A mutations. In some embodiments, the first Fc domain comprises Y349S/K370Y/T366V/K409V mutations, and the second Fc domain comprises E357D/S364Q/Y407A mutations. In some embodiments, the first Fc domain comprises E357D/S364Q/Y407A mutations, and the second Fc domain comprises Y349S/K370Y/T366V/K409V mutations. In some embodiments, the first Fc domain comprises Y349S/K370Y/T366M/K409V mutations, and the second Fc domain comprises E356G/E357D/S364Q/Y407A mutations. In some embodiments, the first Fc domain comprises E356G/E357D/S364Q/Y407A mutations, and the second Fc domain comprises Y349S/K370Y/T366M/K409V mutations. In some embodiments, the first Fc domain comprises Y349S/K370Y/T366M/K409V mutations, and the second Fc domain comprises E357D/S364R/Y407A mutations. In some embodiments, the first Fc domain comprises E357D/S364R/Y407A mutations, and the second Fc domain comprises Y349S/K370Y/T366M/K409V mutations. Each of the foregoing mutations is according to EU numbering.

In some embodiments, the first and second Fe domains comprise DD-KK mutations or related mutations. See, e.g., Gunasekaran K, et al., J Biol Chem (2010) 285(25):19637-46, incorporated herein by reference. In some embodiments, the first Fc domain comprises K409D/K392D mutations, and the second Fc domain comprises D399K/E356K mutations. In some embodiments, the first Fc domain comprises D399K/E356K mutations, and the second Fc domain comprises K409D/K392D mutations. In some embodiments, the first Fc domain comprises K409E/K392D mutations, and the second Fc domain comprises D399R/E356K mutations. In some embodiments, the first Fc domain comprises D399R/E356K mutations, and the second Fc domain comprises K409E/K392D mutations. In some embodiments, the first Fc domain comprises K409D/K392D mutations, and the second Fc domain comprises D399R/E356K mutations. In some embodiments, the first Fc domain comprises D399R/E356K mutations, and the second Fc domain comprises K409D/K392D mutations. In some embodiments, the first Fc domain comprises K409E/K392D mutations, and the second Fc domain comprises D399K/E356K mutations. In some embodiments, the first Fc domain comprises D399K/E356K mutations, and the second Fc domain comprises K409E/K392D mutations. In some embodiments, the first Fc domain comprises K409D/K392D/K370D mutations, and the second Fc domain comprises D399K/E356K/E357K mutations. In some embodiments, the first Fc domain comprises D399K/E356K/E357K mutations, and the second Fc domain comprises K409D/K392D/K370D mutations. Each of the foregoing mutations is according to EU numbering.

In some embodiments, the first and second Fe domains comprise EW-RVT mutations or related mutations. See, e.g., Choi H J, et al., Mol Cancer Ther (2013) 12(12):2748-59 and Choi H J, et al., Mol Immunol (2015) 65(2):377-83, each of which is incorporated herein by reference. In some embodiments, the first Fc domain comprises a K409W mutation, and the second Fc domain comprises F405T/D399V mutations. In some embodiments, the first Fc domain comprises F405T/D399V mutations, and the second Fc domain comprises a K409W mutation. In some embodiments, the first Fc domain comprises K360E/K409W mutations, and the second Fc domain comprises Q347R/D399V/F405T mutations. In some embodiments, the first Fc domain comprises Q347R/D399V/F405T mutations, and the second Fc domain comprises K360E/K409W mutations. In some embodiments, the first Fc domain comprises K360E/K409W/Y349C mutations, and the second Fc domain comprises Q347R/D399V/F405T/S354C mutations. In some embodiments, the first Fc domain comprises Q347R/D399V/F405T/S354C mutations, and the second Fc domain comprises K360E/K409WY349C mutations. Each of the foregoing mutations is according to EU numbering.

In some embodiments, the first and second Fe domains comprise strand-exchange engineered domains (SEED). See, e.g., Davis J H, et al., Protein Eng Des Sel (2010) 23(4):195-202, incorporated herein by reference. SEED CH3 heterodimers are composed of alternating segments of human IgA and IgG CH3 sequences. In some embodiments, the first Fc domain comprises an AG SEED domain and the second Fc domain comprises a GA SEED domain. In some embodiments, the first Fc domain comprises a GA SEED domain and the second Fc domain comprises an AG SEED domain.

In some embodiments, the first and second Fe domains comprise A107 mutations or related mutations. See, e.g., Choi H J, et al. PLoS One (2015) 10(12):e0145349, incorporated herein by reference. In some embodiments, the first Fc domain comprises K370E/K409W mutations, and the second Fc domain comprises E357N/D399V/F405T mutations. In some embodiments, the first Fc domain comprises E357N/D399V/F405T mutations, and the second Fc domain comprises K370E/K409W mutations. In some embodiments, the first Fc domain comprises K370E/K409W mutations, and the second Fc domain comprises E3571/S364T/D399V/F405T mutations. In some embodiments, the first Fc domain comprises E3571/S364T/D399V/F405T mutations, and the second Fc domain comprises K370E/K409W mutations. In some embodiments, the first Fc domain comprises K370M/K409W mutations, and the second Fc domain comprises E357M/S364W/D399V/F405T mutations. In some embodiments, the first Fc domain comprises E357M/S364W/D399V/F405T mutations, and the second Fc domain comprises K370M/K409W mutations. In some embodiments, the first Fc domain comprises K370D/K409W mutations, and the second Fc domain comprises E357M/D399V/F405T mutations. In some embodiments, the first Fc domain comprises E357M/D399V/F405T mutations, and the second Fc domain comprises K370D/K409W mutations. In some embodiments, the first Fc domain comprises E357D/S364W/K370E mutations, and the second Fc domain comprises E357N/K370R mutations. In some embodiments, the first Fc domain comprises E357N/K370R mutations, and the second Fc domain comprises E357D/S364W/K370E mutations. In some embodiments, the first Fc domain comprises E357A/S364Y/K370E mutations, and the second Fc domain comprises E357N/K370H mutations. In some embodiments, the first Fc domain comprises E357N/K370H mutations, and the second Fc domain comprises E357A/S364Y/K370E mutations. In some embodiments, the first Fc domain comprises E357G/S364W/K370E mutations, and the second Fc domain comprises an E357N mutation. In some embodiments, the first Fc domain comprises an E357N mutation, and the second Fc domain comprises E357G/S364W/DK70E mutations. In some embodiments, the first Fc domain comprises E357N/S364W/K370E mutations, and the second Fc domain comprises an E357N mutation. In some embodiments, the first Fc domain comprises an E357N mutation, and the second Fc domain comprises E357N/S364W/DK70E mutations. Each of the foregoing mutations is according to EU numbering.

In some embodiments, the first and second Fe domains comprise Duobody® mutations. See, e.g., Labrijn et al., Nat Biotech 27(8), 767-71 (2009); Labrijn et al., J Immunol 187(6), 3238-46 (2011); Labrijn et al., PNAS 110(13): 5145-5150 (2013); Labrijn et al., Nature Protocols 9(10): 2450-63 (2014); Labrijn et al., Sci Report 7(1) (2017); Engelberts et al. EBioMedicine. (2020) 52:102625 and Middleburg et al Cancers (Basel). (2021) 14; 13(2):287), each of which is incorporated herein by reference in its entirety. Such mutations allow two separately generated antibodies to be recombined into a bispecific antibody by controlled Fab-arm exchange. Non-limiting examples of Duobody® mutations include an F405L mutation and a K409R mutation. In some embodiments, the first Fc domain comprises an F405L mutation. In some embodiments, the first Fc domain comprises a K409R mutation. In some embodiments, the second Fc domain comprises an F405L mutation. In some embodiments, the second Fc domain comprises a K409R mutation. In some embodiments, the first Fc domain comprises an F405L mutation, and the second Fc domain comprises a K409R mutation. In some embodiments, the first Fc domain comprises a K409R mutation, and the second Fc domain comprises an F405L mutation.

In some embodiments, anti-CD3 bispecific antibody comprises CrossMAb® mutations. See, e.g. Surowka et al. MAbs. 202113(1):1967714. CrossMAb® technology addresses the issues of light chain mispairing by swapping the CH1 and CL domains of one of the arms of the bispecific antibody. For example, the heavy chain of one arm of a bispecific antibody can contain, from N-term to C-term, a VH domain, a CL domain, a hinge, a CH2 domain and a CH3 domain, and the light chain of that arm of the bispecific antibody can contain, from N-term to C-term, a VL domain and a CH1 domain. A similar effect can be achieved by swapping the VH and VL domains of one of the arms of the bispecific antibody. For example, the heavy chain of one arm of a bispecific antibody can contain, from N-term to C-term, a VL domain, a CH1 domain, a hinge, a CH2 domain and a CH3 domain, and the light chain of that arm of the bispecific antibody can contain, from N-term to C-term, a VH domain and a CL domain. Additionally, the VH and CH1 of one arm of a bispecific antibody can be swapped with the VL and CL of that arm. For example, the heavy chain of one arm of a bispecific antibody can contain, from N-term to C-term, a VL domain, a CL domain, a hinge, a CH2 domain and a CH3 domain, and the light chain of that arm of the bispecific antibody can contain, from N-term to C-term, a VH domain and a CH1 domain.

In some embodiments, the CH1 and CL domains of the anti-CD3 antibody are swapped. In some embodiments, the VH and VL domains of the anti-CD3 antibody are swapped. In some embodiments, the VH/CH1 domains of the anti-CD3 antibody have been swapped with the VL/CL domains of the anti-CD3 antibody. In some embodiments, the CH1 and CL domains of the anti-tumor antibody are swapped. In some embodiments, the VH and VL domains of the anti-tumor antibody are swapped. In some embodiments, the VH/CH1 domains of the anti-tumor antibody have been swapped with the VL/CL domains of the anti-tumor antibody.

Without being bound by theory, Fe domains with increased binding to the neonatal Fe Receptor (FcRn) at a pH of about 6.0 are believed to have increased circulating half-lives in vivo because they remain bound to the FcRn in the endosomal compartment. When the endosome recycles to the cell surface and opens to the extracellular space, the Fc-containing protein can be released back into the blood, however, increased binding to FcRn at a higher pH (around 7.4) can prevent the release of the Fc-containing protein back into the blood. Accordingly, Fc mutations that increase binding to FcRn at lower pHs, while permitting release from the FcRn at higher pHs should increase their circulating half-lives. See, e.g., Dall' Acqua et al. J. Immunol., 2002, 169:5171-5180. The charge state of histidine changes between pH 6.9 and pH 7.4, and histidine residues in the Fc domain (H310 and H435) where shown to play an important role in the pH-dependent binding to FcRn. Id.; see, also, Oganesyan V et al. J Biol Chem. 2014; 289(11):7812-24). Residues shown to be important in improved FcRn binding include improved P230, F241, V264, T307, N315, A330, A378, N389, M428, and N434 (according to EU numbering). See, e.g., Céline, M. et al., “Selection of IgG Variants with Increased FcRn Binding Using Random and Directed Mutagenesis: Impact on Effector Functions,” Front. Immunol. 2015, vol. 6, doi: 10.3389/fimmu.2015.00039. Exemplary improved FcRn-binding mutations of these residues include, but are not limited to, P230S, V264E, T307A, N315D, A330V, A378V, N389T, M428L, and N434Y. Non-limiting examples of additional improved FcRn-binding mutations known in the art include, 4361, 436V, 311I, 311V, 428L, 434S, 428L/434S, 259I, 308F, 259I/308F, 259I/308F/428L, 307Q/434S, 434A, 434H, 250Q/428L, M252Y/S254T/T256E, 307Q/434A, 307Q/380A/434A, and 308P/434A (according to EU numbering). See, e.g., U.S. Pat. No. 8,637,641, which is incorporated herein by reference in its entirety. In some embodiments, the first Fc domain and/or the second Fc domain are independently engineered to further prolong systemic exposure and increase half-life through enhanced FcRn binding at a lower pH (6.0).

In some embodiments, the first and/or second Fc domain are independently engineered to have improved PK. In some embodiments, the first and/or second Fc domains of the present disclosure independently comprise amino acid substitutions M428L and/or N434S, according to EU numbering. In some embodiments, the first Fc domain comprises the amino acid substitution M428L or N434S. In some embodiments, the first Fc domain comprises amino acid substitutions M428L and N434S. In some embodiments, the first Fc domain comprises the amino acid substitution M428L. In some embodiments, the first Fc domain comprises the amino acid substitution N434S. In some embodiments, the second Fc domain comprises the amino acid substitution M428L or N434S. In some embodiments, the second Fc domain comprises amino acid substitutions M428L and N434S. In some embodiments, the second Fc domain comprises the amino acid substitution M428L. In some embodiments, the second Fc domain comprises the amino acid substitution N434S.

In some embodiments, the first and/or second Fc domain are independently engineered to allow purification of homodimers away from heterodimers by increasing the pI difference between the two monomers. Accordingly, in some embodiments, amino acid substitutions are introduced into either or both of the constant regions of the anti-CD3 antibody and the anti-target antibody to alter the isoelectric point of that antibody. Accordingly, the homodimeric anti-CD3 antibody, the homodimeric anti-target antibody, and the heterodimeric bispecific antibody will each have a different isoelectric point, allowing them to be separated from each other by, e.g., isoelectric focusing. The separation of the heterodimers from the two homodimers can be accomplished if the pIs of the two monomeric antibodies differ by as little as 0.1 pH unit, with 0.2, 0.3, 0.4 and 0.5 or greater all finding use in the present invention. There are two general categories of pI variants: those that increase the pI of the antibody (basic changes) and those that decrease the pI of the antibody (acidic changes). All combinations of these variants are contemplated: one antibody may be wild type, or a variant that does not display a significantly different pI from wild-type, and the other can be either more basic or more acidic. Alternatively, each antibody may be changed, one to more basic and one to more acidic. Such mutations may be introduced into the hinge, CH2 and/or CH3 domains of the antibody.

In some embodiments, the first and/or second Fc domain are independently engineered to allow purification of homodimers away from heterodimers by increasing the pI difference between the two monomers. Accordingly, in some embodiments, amino acid substitutions are introduced into either or both of the constant regions of the anti-CD3 antibody and the anti-tumor antibody to alter the isoelectric point of that antibody. Accordingly, the homodimeric anti-CD3 antibody, the homodimeric anti-tumor antibody, and the heterodimeric bispecific antibody will each have a different isoelectric point, allowing them to be separated from each other by, e.g., isoelectric focusing. The separation of the heterodimers from the two homodimers can be accomplished if the pIs of the two monomeric antibodies differ by as little as 0.1 pH unit, with 0.2, 0.3, 0.4 and 0.5 or greater all finding use in the present invention. There are two general categories of pI variants: those that increase the pI of the antibody (basic changes) and those that decrease the pI of the antibody (acidic changes). All combinations of these variants are contemplated: one antibody may be wild type, or a variant that does not display a significantly different pI from wild-type, and the other can be either more basic or more acidic. Alternatively, each antibody may be changed, one to more basic and one to more acidic. Such mutations may be introduced into the hinge, CH2 and/or CH3 domains of the antibody.

In some embodiments, pI mutations are introduced into the hinge domain. Residues 221, 222, 223, 224, 225, 233, 234, 235 and 236 (according to EU numbering) within the hinge may be mutated to alter the pI of an antibody. Non-limiting examples of substitutions that may be used to lower the pI of hinge domains include a deletion at position 221, a non-native valine or threonine at position 222, a deletion at position 223, a non-native glutamic acid at position 224, a deletion at position 225, a deletion at position 235 and a deletion or a non-native alanine at position 236.

In some embodiments, pI mutations are introduced into the CH2 domain. Residues 274, 296, 300, 309, 320, 322, 326, 327, 334 and 339 (according to EU numbering) within the CH2 may be mutated to alter the pI of an antibody. Non-limiting examples of substitutions that may be used to lower the pI of CH2 domains include a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339.

In some embodiments, pI mutations are introduced into the CH3 domain. Residues 355, 359, 362, 384, 389, 392, 397, 418, 419, 444 and 447 (according to EU numbering) within the CH3 may be mutated to alter the pI of an antibody. Non-limiting examples of substitutions that may be used to lower the pI of CH3 domains include a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

Mutations that delete a positively charged residue or substitute it with a neutral or negatively charged residue will lower the pI of an antibody. Non-limiting examples of positively charged residues that may be deleted or substituted include: K274, R355, K392, and K447 (according to EU numbering). Exemplary pI-lowering mutations of these residues include: K274Q, R355Q, K392N, and K447del (deletion of K447). Mutations that substitute a neutral residue with a negatively charged residue will also lower the pI of an antibody. Non-limiting examples of neutral residues that may be substituted with negatively charged residues include: N203, Q295, N384, Q418, Q419, and N421 (according to EU numbering). Exemplary pI-lowering mutations of these residues include: N203D, Q295E, N384D, Q418E, Q419E, and N421D.

Mutations that delete a negatively charged residue or substitute it with a neutral or positively charged residue will raise the pI of an antibody. Non-limiting examples of negatively charged residues that may be deleted or substituted include: E269, E272, E283, and E357 (according to EU numbering). Exemplary pI-raising mutations of these residues include: E269Q, E272Q, E283Q, and E357Q. Mutations that substitute a neutral residue with a positively charged residue will also raise the pI of an antibody. Non-limiting examples of neutral residues that may be substituted with positively charged residues include: Q196, P217, P228, and N276 (according to EU numbering). Exemplary pI-raising mutations of these residues include: Q196K, P217R, P228R, and N276K.

In some embodiments, the first Fc domain comprises one or more mutations to lower the isoelectric point as compared to a wild-type Fc domain. In some embodiments, the first Fc domain comprises one or more mutations to raise the isoelectric point as compared to a wild-type Fc domain. In some embodiments, the second Fc domain comprises one or more mutations to lower the isoelectric point as compared to a wild-type Fc domain. In some embodiments, the second Fc domain comprises one or more mutations to raise the isoelectric point as compared to a wild-type Fc domain. In some embodiments, the first Fc domain comprises one or more mutations to lower the isoelectric point as compared to a wild-type Fc domain and the second Fc domain comprises one or more mutations to raise the isoelectric point as compared to a wild-type Fc domain. In some embodiments, the first Fc domain comprises one or more mutations to raise the isoelectric point as compared to a wild-type Fc domain and the second Fc domain comprises one or more mutations to lower the isoelectric point as compared to a wild-type Fc domain.

In some embodiments, the anti-tumor antibody is an anti-CD33 antibody. In some embodiments, the anti-CD33 antibody comprises the six CDRs of gemtuzumab. In some embodiments, the anti-CD33 antibody comprises the VH of gemtuzumab. In some embodiments, the anti-CD33 antibody comprises the VL of gemtuzumab. In some embodiments, the anti-CD33 antibody comprises the VH and the VL of gemtuzumab. In some embodiments, the heavy chain of the anti-CD33 antibody comprises the amino acid sequence of SEQ ID NO: 1150. In some embodiments, the light chain of the anti-CD33 antibody comprises the amino acid sequence of SEQ ID NO: 1151. In some embodiments, the heavy chain of the anti-CD33 antibody comprises the amino acid sequence of SEQ ID NO: 1150, and the light chain of the anti-CD33 antibody comprises the amino acid sequence of SEQ ID NO: 1151.

### Polynucleotides

One aspect of the present disclosure provides a polynucleotide sequence that encodes an anti-CD3 antibody, or antigen-binding fragment thereof, disclosed herein. In some embodiments, the anti-CD3 antibody is an anti-CD3 bispecific antibody. In some embodiments, the polynucleotide is a recombinant polynucleotide. In some embodiments, the polynucleotide is cDNA. In some embodiments, separate polynucleotide molecules encode a heavy chain and a light chain of an anti-CD3 antibody or fragment. In some embodiments, a first polynucleotide encodes the heavy chain of an anti-CD3 antibody or fragment, and a second polynucleotide encodes the light chain of the anti-CD3 antibody or fragment. In other embodiments, the same polynucleotide molecule encodes both a heavy chain and a light chain of an anti-CD3 antibody or fragment.

In some embodiments, the polynucleotide encodes a CDR of an anti-CD3 antibody disclosed herein. In some embodiments, the polynucleotide comprises a sequence encoding a HCDR1 polynucleotide sequence disclosed herein. In some embodiments, the polynucleotide comprises a sequence encoding a HCDR2 polynucleotide sequence disclosed herein. In some embodiments, the polynucleotide comprises a sequence encoding a HCDR3 polynucleotide sequence disclosed herein. In some embodiments, the polynucleotide comprises a sequence encoding a LCDR1 polynucleotide sequence disclosed herein. In some embodiments, the polynucleotide comprises a sequence encoding a LCDR2 polynucleotide sequence disclosed herein. In some embodiments, the polynucleotide comprises a sequence encoding a LCDR3 polynucleotide sequence disclosed herein. In some embodiments, the polynucleotide comprises a sequence encoding the six CDRs of an anti-CD3 antibody disclosed herein. In some embodiments, the polynucleotide comprises a sequence encoding the LCDR1, LCDR2, LCDR3, HCDR1, HCDR2, and HCDR3 of an anti-CD3 antibody disclosed herein.

In some embodiments, a polynucleotide comprises a sequence encoding a VH of an anti-CD3 antibody disclosed herein. In some embodiments, a polynucleotide comprises a sequence encoding a VL of an anti-CD3 antibody disclosed herein. In some embodiments, a polynucleotide comprises a sequence encoding a VH of an anti-CD3 antibody disclosed herein and a sequence encoding a VL of an anti-CD3 antibody disclosed herein.

In some embodiments, the polynucleotide encodes a VL comprising an amino acid sequence that is at least 70%, 75%, 80%, 85%, 90%, 95%, 97%, 98% or 99% identical to the VL amino acid sequence of any one of SEQ ID NOs: 688-914. Optionally, the polynucleotide encodes a VL comprising an amino acid sequence that is at least 70%, 75%, 80%, 85%, 90%, 95%, 97%, 98% or 99% similar to the VL amino acid sequence of SEQ ID NO: 688-914. Polynucleotides may include nucleotide sequences that hybridize under highly stringent conditions to the complement of a polynucleotide encoding the amino acid sequence the VL region of SEQ ID NO: 688-914.

In some embodiments, the polynucleotide encodes a VH comprising an amino acid sequence that is at least 70%, 75%, 80%, 85%, 90%, 95%, 97%, 98% or 99% identical to the VH amino acid sequence of any one of SEQ ID NOs: 915-1141. Optionally, the polynucleotide encodes a VH comprising an amino acid sequence that is at least 70%, 75%, 80%, 85%, 90%, 95%, 97%, 98% or 99% similar to the VH amino acid sequence of SEQ ID NO: 915-1141. Polynucleotides may include nucleotide sequences that hybridize under highly stringent conditions to the complement of a polynucleotide encoding the amino acid sequence the VH region of SEQ ID NO: 915-1141.

It is known in the art that each heavy chain and light chain (or fragment thereof) is expressed comprising a leader sequence (also known as a signal sequence) at its N terminus, which is used to transport the newly synthesized chains into the endoplasmic reticulum. During post-translational processing the leader sequences is remove and, therefore, is not present in the final chain or the mature antibody. Most heavy chain leader sequences contain 19 amino acid residues (i.e., are encoded by the first 57 nucleotides), and most light chain leader sequences contain 22 amino acid residues (i.e. are encoded by the first 66 nucleotides). Haryadi R, et al., PLoS One, 2015, vol. 10(2): e0116878. The skilled artisan would recognize that the nucleotide sequence encoding the mature heavy chain or light chain (or fragment thereof) may be operably linked to a particular leader sequence to optimize its expression in a given expression system. Id.

### Vectors

An additional aspect of the disclosure provides one or more vectors comprising a polynucleotide sequence encoding an anti-CD3 antibody, or an antigen-binding fragment thereof, disclosed herein. In some embodiments, the anti-CD3 antibody is an anti-CD3 bispecific antibody. In some embodiments, the sequences encoding the heavy and light chains of the anti-CD3 antibody or fragment are on the same vector. In some embodiments, the sequences encoding the heavy and lights chain of the anti-CD3 antibody or fragment are on separate vectors. In some embodiments, the sequence encoding the heavy chain of the anti-CD3 antibody or fragment is on a first vector and the sequence encoding the light chain of the anti-CD3 antibody or fragment is on a second vector. In some embodiments, the vector comprising a polynucleotide sequence encoding a HCDR1, a HCDR2, and an HCDR3 of an anti-CD3 antibody, or an antigen-binding fragment thereof, disclosed herein. The vector may comprise a polynucleotide sequence encoding a LCDR1, a LCDR2, and an LCDR3 of an anti-CD3 antibody, or an antigen-binding fragment thereof, disclosed herein. In some embodiments, the vector comprising a polynucleotide sequence encoding a HCDR1, a HCDR2, an HCDR3, a LCDR1, a LCDR2, and an LCDR3 of an anti-CD3 antibody, or an antigen-binding fragment thereof, disclosed herein. In some embodiments, the vector comprising a polynucleotide sequence encoding a VH of an anti-CD3 antibody, or an antigen-binding fragment thereof, disclosed herein. The vector may comprise a polynucleotide sequence encoding a VL of an anti-CD3 antibody, or an antigen-binding fragment thereof, disclosed herein. In some embodiments, the vector comprising a polynucleotide sequence encoding a VL and a VH of an anti-CD3 antibody, or an antigen-binding fragment thereof, disclosed herein. In some embodiments, the vector comprising a polynucleotide sequence encoding a heavy chain of an anti-CD3 antibody, or an antigen-binding fragment thereof, disclosed herein. The vector may comprise a polynucleotide sequence encoding a light chain of an anti-CD3 antibody, or an antigen-binding fragment thereof, disclosed herein. In some embodiments, the vector comprising a polynucleotide sequence encoding a light chain and a heavy chain of an anti-CD3 antibody, or an antigen-binding fragment thereof, disclosed herein. In some embodiments, the vector is an expression vector. In some embodiments, the vector is a cloning vector.

Suitable cloning and expression vectors can include a variety of components, such as promoter, enhancer, and other transcriptional regulatory sequences. The vector may also be constructed to allow for subsequent cloning of an antibody variable domain into different vectors.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColEl, pCR1, RP4, pCDNA, TGEX phage DNAs, and shuttle vectors such as pSA3 and pAT28. These and many other cloning vectors are available from commercial vendors such as Thermo, BioRad, Agilent, and Antibody Design Labs.

Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, stop codons, and polyadenylation signals.

The vectors containing the polynucleotides of the disclosure and/or the polynucleotides themselves, can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The antibody, or antigen-binding fragment thereof, may be made recombinantly using a suitable host cell. A nucleic acid encoding the antibody or antigen-binding fragment thereof can be cloned into an expression vector, which can then be introduced into a host cell to obtain the synthesis of an antibody in the recombinant host cell.

### Host Cells

An additional aspect of the disclosure provides a host cell for expressing an anti-CD3 antibody or an antigen-binding fragment thereof of the disclosure. In some embodiments, the anti-CD3 antibody is an anti-CD3 bispecific antibody. In some embodiments, the host cell comprises a polynucleotide encoding the heavy and light chains of an anti-CD3 antibody or fragment disclosed herein. In some embodiments, the host cell comprises a pair of polynucleotides: one encoding the light chain and the other encoding the heavy chain of an anti-CD3 antibody disclosed herein. In some embodiments, the host cell comprises a vector encoding the heavy and light chains of an anti-CD3 antibody or fragment disclosed herein. In some embodiments, the host cell comprises a pair of vectors: one encoding the light chain and the other encoding the heavy chain of an anti-CD3 antibody or fragment disclosed herein. In some embodiments, the host cell is an isolated host cell.

In some embodiments, the host cell is an E. coli cell, a yeast cell, an insect cell, a simian COS cell, a Chinese hamster ovary (CHO) cell, a myeloma cell, or a hybridoma cell. In some embodiments, the host cell is a mammalian cell. Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines of particular preference are selected through determining which cell lines have high expression levels. In some embodiments, the host cell is a CHO cell, a Human embryonic kidney (HEK) 293 cell, or an Sp2.0 cell. In some embodiments, the host cell is a CHO cell. In some embodiments, the host cell is a HEK293 cell. In some embodiments, the host cell is an Sp2.0 cell. In some embodiments, the host cell is a hybridoma cell. Other cell lines that may be used are insect cell lines, such as Sf9 cells. In some embodiments, the host cell does not otherwise produce an immunoglobulin protein.

### Methods of Producing Anti-CD3 Antibodies

An additional aspect of the disclosure provides a method for producing an anti-CD3 antibody or an antigen-binding fragment thereof of the disclosure. In some embodiments, the anti-CD3 antibody is an anti-CD3 bispecific antibody. The antibody, or antigen-binding fragment thereof, may be made recombinantly using a suitable host cell. A nucleic acid encoding the antibody or antigen-binding fragment thereof can be cloned into an expression vector, which can then be introduced into a host cell to obtain the synthesis of an antibody in the recombinant host cell.

When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods. In some embodiments, the method comprises culturing a host cell disclosed herein under conditions in which the antibody or fragment is expressed by the cell. In some embodiments, the method further comprises recovering the antibody or fragment from the culture media.

### Modified Anti-CD3 Antibodies

Anti-CD3 antibodies of the present disclosure, and antigen-binding fragments and variants thereof, may also be conjugated or operably linked to another compound (e.g., therapeutic agent, label, or tag), referred to herein as a conjugate. In some embodiments, the anti-CD3 antibody is an anti-CD3 bispecific antibody. The conjugate may be a cytotoxic agent, a chemotherapeutic agent, a cytokine, an anti-angiogenic agent, a tyrosine kinase inhibitor, a toxin, a radioisotope, or other therapeutically active agent. Chemotherapeutic agents, cytokines, anti-angiogenic agents, tyrosine kinase inhibitors, and other therapeutic agents are contemplated. In some embodiments, the antibody is conjugated or operably linked to a toxin, including but not limited to small molecule toxins and enzymatically active toxins of bacterial, fungal, plant, animal or synthetic origin, including fragments and/or variants thereof.

There are many linking groups known in the art for making antibody conjugates, including, for example, those disclosed in U.S. Pat. No. 5,208,020 or EP Patent 0425235 B1, and Chari et al., Cancer Research 52: 127-131 (1992). The linking groups include disulfide groups, thioether groups, acid labile groups, photolabile groups, peptidase labile groups, or esterase labile groups, as disclosed in the above-identified patents, disulfide and thioether groups being preferred.

### Pharmaceutical Compositions

An additional aspect of the present disclosure provides pharmaceutical compositions and methods of use. The pharmaceutical compositions of the present disclosure include an anti-CD3 antibody, or fragment thereof, disclosed herein in a pharmaceutically acceptable carrier. In some embodiments, the anti-CD3 antibody is an anti-CD3 bispecific antibody. Pharmaceutical compositions may be administered in vivo for the treatment or prevention of a disease or disorder. Furthermore, pharmaceutical compositions comprising an antibody, or a fragment thereof, of the present disclosure may include one or more agents for use in combination or may be administered in conjunction with one or more agents. Agents for use in combination with an anti-CD3 antibody disclosed herein include, but are not limited to cytotoxic agents, chemotherapeutic agents, cytokines, anti-angiogenic agents, tyrosine kinase inhibitors, toxins, and radioisotopes.

The pharmaceutical compositions disclosed herein may include a therapeutically effective amount or a prophylactically effective amount of an antibody disclosed herein. A therapeutically effective amount of the antibody may vary according to factors such as the disease state, age, sex, and weight of the individual, and the ability of the antibody or antibody portion to elicit a desired response in the individual. A therapeutically effective amount is also one in which any toxic or detrimental effects of the antibody are outweighed by the therapeutically beneficial effects. It is routine in the art for the skilled artisan to determine a therapeutically effective amount of an antibody disclosed herein based on these factors.

The present disclosure also provides kits relating to any of the antibodies, or fragments thereof, and/or methods disclosed herein. In some embodiments, the anti-CD3 antibody is an anti-CD3 bispecific antibody. Kits of the present disclosure may include diagnostic or therapeutic agents. A kit of the present disclosure may further provide instructions for use of a composition or antibody and packaging. A kit of the present disclosure may include devices, reagents, containers or other components. Furthermore, a kit of the present disclosure may also require the use of an apparatus, instrument or device, including a computer.

### Methods of Use

The AlivaMab® antibodies against CD3, and in particular fully human antibodies and bispecific antibodies incorporating all or portions of the heavy chain and light chain variable regions from the AlivaMab® antibodies, may have utility for the treatment of human disease including, but not limited to, cancer and autoimmunity.

An anti-CD3 antibody, or antigen-binding fragment thereof, disclosed herein may be used in research, diagnostic, and/or therapeutic methods. In some embodiments, an anti-CD3 antibody, or antigen-binding fragment thereof, disclosed herein is used to treat cancer. In some embodiments, the method comprising administering a therapeutically effective amount of an antibody, or antigen-binding fragment thereof, disclosed herein. In some embodiments, the method comprising administering a therapeutically effective amount of a bispecific antibody, or antigen-binding fragment thereof, disclosed herein. In some embodiments, the anti-CD3 antibody is an anti-CD3 bispecific antibody comprising an anti-tumor antibody.

In some embodiments, an anti-CD3 antibody, or antigen-binding fragment thereof, disclosed herein is used to treat an autoimmune disease. In some embodiments, the autoimmune disease is graft-versus-host disease or type I diabetes as well as T-cell mediated autoimmune disorders. In some embodiments, the method comprising administering a therapeutically effective amount of an antibody, or antigen-binding fragment thereof, disclosed herein.

A further aspect of the disclosure provides methods of treating or preventing cancer in a subject in need thereof by administering an anti-CD3 antibody, or antigen-binding fragment thereof, of the disclosure or a composition comprising the antibody or fragment to a subject in need thereof. An additional aspect of the disclosure provides an anti-CD3 antibody, or an antigen-binding fragment thereof, of the disclosure for use in the treatment of cancer. Another aspect of the disclosure provides use of an anti-CD3 antibody, or an antigen-binding fragment thereof, of the disclosure in the manufacture of a medicament for treating cancer. In some embodiments, the anti-CD3 antibody is an anti-CD3 bispecific antibody. In some embodiments, the anti-CD3 antibody is an anti-CD3 multi-specific antibody.

Another aspect of the disclosure provides methods of treating or preventing autoimmune diseases or disorders in a subject in need thereof by administering an anti-CD3 antibody, or antigen-binding fragment thereof, of the disclosure or a composition comprising the antibody or fragment to a subject in need thereof. An additional aspect of the disclosure provides an anti-CD3 antibody, or an antigen-binding fragment thereof, of the disclosure for use in the treatment of autoimmune diseases or disorders. Another aspect of the disclosure provides use of an anti-CD3 antibody, or an antigen-binding fragment thereof, of the disclosure in the manufacture of a medicament for treating autoimmune diseases or disorders. In some embodiments, the anti-CD3 antibody is an anti-CD3 bispecific antibody. In some embodiments, the anti-CD3 antibody is an anti-CD3 multi-specific antibody.

One aspect of the disclosure provides a method of maintaining cancer remission in a subject by administering an anti-CD3 antibody, or antigen-binding fragment thereof, of the disclosure or a composition comprising the antibody or fragment to a subject in need thereof. An additional aspect of the disclosure provides an anti-CD3 antibody, or an antigen-binding fragment thereof, of the disclosure for use in maintaining cancer remission. Another aspect of the disclosure provides use of an anti-CD3 antibody, or an antigen-binding fragment thereof, of the disclosure in the manufacture of a medicament for maintaining cancer remission. In some embodiments, the anti-CD3 antibody is an anti-CD3 bispecific antibody. In some embodiments, the anti-CD3 antibody is an anti-CD3 multi-specific antibody.

An additional aspect of the disclosure provides a method of redirecting a T-cell to a tumor cell, wherein the method comprises contacting the T-cell with an anti-CD3 bispecific antibody, or a bispecific antigen-binding fragment thereof, of the disclosure or a composition comprising the bispecific antibody or fragment. A further aspect of the disclosure provides an anti-CD3 bispecific antibody, or a bispecific antigen-binding fragment thereof, for use in targeting a T-cell to a tumor cell. One aspect of the disclosure provides the use of an anti-CD3 bispecific antibody, or a bispecific antigen-binding fragment thereof, in the manufacture of a medicament for targeting a T-cell to a tumor cell. In some embodiments, bispecific antibody or fragment further comprises a tumor-targeting arm.

A further aspect of the invention provides a method of treating cancer by administering an anti-CD3 bispecific antibody, or a bispecific antigen-binding fragment thereof, of the disclosure or a composition comprising administering the bispecific antibody or fragment to a subject in need thereof, wherein the bispecific antibody or fragment further comprises a tumor-targeting arm with an anti-tumor antibody. An additional aspect of the disclosure provides an anti-CD3 bispecific antibody, or a bispecific antigen-binding fragment thereof, of the disclosure or a composition comprising the bispecific antibody or fragment for use in treating cancer, wherein the bispecific antibody or fragment further comprises a tumor-targeting arm. One aspect of the disclosure provides use of an anti-CD3 bispecific antibody, or a bispecific antigen-binding fragment thereof, of the disclosure in the manufacture of a medicament for treating cancer.

### EXEMPLARY EMBODIMENTS

Particular embodiments of the disclosure are set forth in the following numbered paragraphs:
1. An isolated anti-CD3 antibody, or an antigen-binding fragment thereof, comprising:
   i) a heavy chain variable region comprising a VHCDR1 selected from any of SEQ ID NOs: 345-457, a VHCDR2 selected from any of SEQ ID NOs: 458-573, and a VHCDR3 selected from any of SEQ ID NOs: 574-687 and
   ii) a light chain variable region comprising a VLCDR1 selected from any of SEQ ID NOs:1-113, a VLCDR2 selected from any of SEQ ID NOs: 114-226, and a VLCDR3 selected from any of SEQ ID NOs:227-377.
2. The antibody, or antigen-binding fragment thereof, of paragraph 1, wherein the VH is selected from any one of SEQ ID NOs: 915-1141.
3. The antibody, or antigen-binding fragment thereof, of paragraph 1 or paragraph 2, wherein the VL is selected from any one of SEQ ID NOs: 688-914.
4. The antibody, or antigen-binding fragment thereof, of any one of paragraphs 1-3, wherein the antibody is human.
5. The antibody, or antigen-binding fragment thereof, of any one of paragraphs 1-3, wherein the antibody is chimeric.
6. The antibody, or antigen-binding fragment thereof, of any one of paragraphs 1-5, wherein the antibody is selected from a single-variable domain antibody, single chain antibody, a scFv, a bispecific antibody, a multi-specific antibody, a Fab, a F(ab′)2, and a whole antibody.
7. The antibody, or antigen-binding fragment thereof, of paragraph 6, wherein the antibody is a multi-specific antibody.
8. The antibody, or antigen-binding fragment thereof, of paragraph 6, wherein the antibody is a bispecific antibody comprising first and second Fc domains.
9. The antibody, or antigen-binding fragment thereof, of paragraph 8, wherein the bispecific antibody further comprises a targeting antibody.
10. The antibody, or antigen-binding fragment thereof, of paragraph 9, wherein the targeting antibody is an anti-tumor antibody, or an antigen-binding fragment thereof.
11. The antibody, or antigen-binding fragment thereof, of any one of paragraphs paragraph 6-10, wherein the anti-CD3 antibody, or the antigen-binding fragment thereof, comprises an scFv.
12. The antibody, or antigen-binding fragment thereof, of paragraph 6 or paragraph 11, wherein the anti-CD3 scFv antibody comprises an amino acid sequence selected from any one of SEQ ID NOs: 1155-1236.
13. The antibody, or antigen-binding fragment thereof, of paragraph 11 or 12, wherein the anti-CD3 scFv antibody is operably linked to the first Fc domain.
14. The antibody, or antigen-binding fragment thereof, of paragraph 13, wherein the anti-CD3 scFv antibody is operably linked to the first Fc domain through a first linker.
15. The antibody, or antigen-binding fragment thereof, of any one of paragraphs 9-14, wherein the targeting antibody, or the antigen-binding fragment thereof, comprises a Fab fragment or a F(ab′)2 fragment.
16. The antibody, or antigen-binding fragment thereof, of paragraph 15, wherein the targeting Fab or F(ab′)2 fragment is operably linked to the second Fc domain.
17. The antibody, or antigen-binding fragment thereof, of paragraph 16, wherein the targeting Fab or F(ab′)2 fragment is operably linked to the second Fc domain through a second linker.
18. The antibody, or antigen-binding fragment thereof, of paragraph 15 or 17, wherein the first linker or second linker is a variable length Gly-Ser linker.
19. The antibody, or antigen-binding fragment thereof, of paragraph 18, wherein the first linker and second linker are independently selected from SEQ ID NOs.: 1146-1149, and 1237-1238.
20. The antibody, or antigen-binding fragment thereof, of any one of paragraphs 8-19, wherein the first and second Fc domain comprises complementary mutations selected from knobs-into-holes (KIH) mutations, K409R/F405L, HA-TF and related mutations, skew mutations, ZW mutations, 7.8.60 mutations or related mutations, DD-KK mutations or related mutations, EW-RVT mutations or related mutations, strand-exchange engineered domains (SEED), Duobody mutations, CrossMAb mutations, and A107 mutations or related mutations.
21. The antibody, or antigen-binding fragment thereof, of paragraph 20, wherein (1) the first Fc domain comprises a T366W mutation and the second Fc domain comprises T366S/L368A/Y407V mutations; (2) the first Fc domain comprises T366S/L368A/Y407V mutations and the second Fc domain comprises a T366W mutation; (3) the first Fc domain comprises S354C/T366W mutations and the second Fc domain comprises Y349C/T366S/L368A/Y407V mutations; (4) the first Fc domain comprises Y349C/T366S/L368A/Y407V mutations and the second Fc domain comprises S354C/T366W mutation; (5) the first Fc domain comprises S354C/T366W mutations and the second Fc domain comprises Y349C/T366S/L368A/Y407V/H435R/Y436F mutations; (6) the first Fc domain comprises Y349C/T366S/L368A/Y407V/H435R/Y436F mutations and the second Fc domain comprises S354C/T366W mutations; 7) the first Fc domain comprises S354C/T366W/H435R/Y436F mutations and the second Fc domain comprises Y349C/T366S/L368A/Y407V mutations; or 8) the first Fc domain comprises Y349C/T366S/L368A/Y407V mutations and the second Fc domain comprises S354C/T366W/H435R/Y436F mutations.
22. The antibody, or antigen-binding fragment thereof, of paragraph 20, wherein (1) the first and second Fe domains comprise the amino acid sequences of SEQ ID NOs: 1142 and 1143, respectively; or (2) the first and second Fc domains comprise the amino acid sequences of SEQ ID NOs: 1143 and 1142, respectively.
23. The antibody, or antigen-binding fragment thereof, of paragraph 20, wherein (1) the anti-CD3 antibody comprises a constant region comprising the amino acid sequence of SEQ ID NO: 1144, and the anti-tumor antibody comprises a constant region comprising the amino acid sequence of SEQ ID NO: 1145; or (2) the anti-CD3 antibody comprises a constant region comprising the amino acid sequence of SEQ ID NO: 1145, and the anti-tumor antibody comprises a constant region comprising the amino acid sequence of SEQ ID NO: 1144.
24. The antibody, or antigen-binding fragment thereof, of paragraph 20, wherein (1) the first Fc domain comprises a S364H mutation and the second Fc domain comprises a Y349T mutation; (2) the first Fc domain comprises a Y349T mutation and the second Fc domain comprises a S364H mutation; (3) the first Fc domain comprises a F405A mutation and the second Fc domain comprises a T394F mutation; (4) the first Fc domain comprises a T394F mutation and the second Fc domain comprises a F405A mutation; (5) the first Fc domain comprises S364H/T394F mutations and the second Fc domain comprises Y349T/F405A mutations; (6) the first Fc domain comprises Y349T/F405A mutations and the second Fc domain comprises S364H/T394F mutations; (7) the first Fc domain comprises S364H/F405A mutations and the second Fc domain Y349T/T394F mutations; or (8) the first Fc domain comprises Y349T/T394F mutations and the second Fc domain S364H/F405A mutations.
25. The antibody, or antigen-binding fragment thereof, of paragraph 20, wherein (1) the first Fc domain comprises S364K/E357Q mutations and the second Fc domain comprises L368D/K370S mutations; (2) the first Fc domain comprises L368D/K370S mutations and the second Fc domain comprises S364K/E357Q mutations; (3) the first Fc domain comprises L368D/K370S mutations and the second Fc domain comprises a S364K mutation; (4) the first Fc domain comprises a S364K mutation and the second Fc domain comprises L368D/K370S mutations; (5) the first Fc domain comprises L368E/K370S mutations and the second Fc domain comprises a S364K mutation; (6) the first Fc domain comprises a S364K mutation and the second Fc domain comprises L368E/K370S mutations; (7) the first Fc domain comprises T411E/K360E/Q362E mutations and the second Fc domain comprises a D401K mutation; (8) the first Fc domain comprises a D401K mutation and the second Fc domain comprises T411E/K360E/Q362E mutations; (9) the first Fc domain comprises L368D/K370S mutations and the second Fc domain comprises S364K/E357L mutations; (10) the first Fc domain comprises S364K/E357L mutations and the second Fc domain comprises L368D/K370S mutations; (11) the first Fc domain comprises a K370S mutation and the second Fc domain comprises S364K/E357Q mutations; (12) the first Fc domain comprises S364K/E357Q mutations and the second Fc domain comprises a K370S mutation; (13) the first Fc domain comprises T366S/L368A/Y407V mutations and the second Fc domain comprises a T366W mutation; (14) the first Fc domain comprises a T366W mutation and the second Fc domain comprises T366S/L368A/Y407V mutations; (15) the first Fc domain comprises T366S/L368A/Y407V/Y349C mutations and the second Fc domain comprises T366W/S354C mutations; or (16) the first Fc domain comprises T366W/S354C mutations and the second Fc domain comprises T366S/L368A/Y407V/Y349C mutations.
26. The antibody, or antigen-binding fragment thereof, of paragraph 20, wherein (1) the first Fc domain comprises F405A/Y407V mutations and the second Fc domain comprises a T394W mutation; (2) the first Fc domain comprises a T394W mutation and the second Fc domain comprises F405A/Y407V mutations; (3) the first Fc domain comprises F405A/Y407V mutations and the second Fc domain comprises T366I/T394W mutations; (4) the first Fc domain comprises T366I/T394W mutations and the second Fc domain comprises F405A/Y407V mutations; (5) the first Fc domain comprises F405A/Y407V mutations and the second Fc domain comprises T366L/T394W mutations; (6) the first Fc domain comprises T366L/T394W mutations and the second Fc domain comprises F405A/Y407V mutations; (7) the first Fc domain comprises F405A/Y407V mutations and the second Fc domain comprises T366L/K392M/T394W mutations; (8) the first Fc domain comprises T366L/K392M/T394W mutations and the second Fc domain comprises F405A/Y407V mutations; (9) the first Fc domain comprises L351Y/F405A/Y407V mutations and the second Fc domain comprises T366L/K329M/T394W mutations; (10) the first Fc domain comprises T366L/K329M-/T394W mutations and the second Fc domain comprises L351Y/F405A/Y407V mutations; (11) the first Fc domain comprises T350V/L351Y/F405A/Y407V mutations and the second Fc domain comprises T350V/T366L/K392M/T394W mutations; (12) the first Fc domain comprises T350V/T366L/K392M/T394W mutations and the second Fc domain comprises T350V/L351Y/F405A/Y407V mutations; (13) the first Fc domain comprises T350V/L351Y/F405A/Y407V mutations and the second Fc domain comprises T350V/T366L-/K392L/T394W mutations; or (14) the first Fc domain comprises T350V/T366L/K392L-/T394W mutations and the second Fc domain comprises T350V/L351Y/F405A/Y407V mutations.
27. The antibody, or antigen-binding fragment thereof, of paragraph 20, wherein (1) the first Fc domain comprises D399M/Y407A mutations and the second Fc domain comprises T366V/K409V mutations; (2) the first Fc domain comprises T366V/K409V mutations and the second Fc domain comprises D399M/Y407A mutations; (3) the first Fc domain comprises K360D/D399M/Y407A mutations and the second Fc domain comprises E345R/Q347R/T366V/K409V mutations; (4) the first Fc domain comprises E345R/Q347R/T366V/K409V mutations and the second Fc domain comprises K360D/D399M/Y407A mutations; (5) the first Fc domain comprises Y349S/K370Y/T366V-/K409V mutations and the second Fc domain comprises E357D/S364Q/Y407A mutations; (6) the first Fc domain comprises E357D/S364Q/Y407A mutations and the second Fc domain comprises Y349S/K370Y/T366V/K409V mutations; (7) the first Fc domain comprises Y349S/K370Y/T366M/K409V mutations, and the second Fc domain comprises E356G/E357D/S364Q/Y407A mutations; (8) the first Fc domain comprises E356G/E357D/S364Q/Y407A mutations, and the second Fc domain comprises Y349S/K370Y/T366M/K409V mutations; (9) the first Fc domain comprises Y349S/K370Y/T366M/K409V mutations and the second Fc domain comprises E357D/S364R/Y407A mutations; or (10) the first Fc domain comprises E357D/S364R/Y407A mutations and the second Fc domain comprises Y349S/K370Y/T366M/K409V mutations.
28. The antibody, or antigen-binding fragment thereof, of paragraph 20, wherein (1) the first Fc domain comprises K409D/K392D mutations and the second Fc domain comprises D399K/E356K mutations; (2) the first Fc domain comprises D399K/E356K mutations and the second Fc domain comprises K409D/K392D mutations; (3) the first Fc domain comprises K409E/K392D mutations and the second Fc domain comprises D399R/E356K mutations; (4) the first Fc domain comprises D399R/E356K mutations and the second Fc domain comprises K409E/K392D mutations; (5) the first Fc domain comprises K409D/K392D mutations and the second Fc domain comprises D399R/E356K mutations; (6) the first Fc domain comprises D399R/E356K mutations and the second Fc domain comprises K409D/K392D mutations; (7) the first Fc domain comprises K409E/K392D mutations and the second Fc domain comprises D399K/E356K mutations; (8) the first Fc domain comprises D399K/E356K mutations and the second Fc domain comprises K409E/K392D mutations; (9) the first Fc domain comprises K409D/K392D/K370D mutations and the second Fc domain comprises D399K/E356K/-E357K mutations; or (10) the first Fc domain comprises D399K/E356K/E357K mutations and the second Fc domain comprises K409D/K392D/K370D mutations.
29. The antibody, or antigen-binding fragment thereof, of paragraph 20, wherein (1) the first Fc domain comprises a K409W mutation and the second Fc domain comprises F405T/D399V mutations; (2) the first Fc domain comprises F405T/D399V mutations and the second Fc domain comprises a K409W mutation; (3) the first Fc domain comprises K360E/K409W mutations and the second Fc domain comprises Q347R/D399V/F405T mutations; (4) the first Fc domain comprises Q347R/D399V/F405T mutations and the second Fc domain comprises K360E/K409W mutations; (5) the first Fc domain comprises K360E/K409W/Y349C mutations and the second Fc domain comprises Q347R/D399V/F405T/S354C mutations; or (6) the first Fc domain comprises Q347R/D399V/F405T/S354C mutations and the second Fc domain comprises K360E/K409WY349C mutations.
30. The antibody, or antigen-binding fragment thereof, of paragraph 20, wherein (1) the first Fc domain comprises an AG SEED domain and the second Fc domain comprises a GA SEED domain or (2) the first Fc domain comprises a GA SEED domain and the second Fc domain comprises an AG SEED domain.
31. The antibody, or antigen-binding fragment thereof, of paragraph 20, wherein (1) the first Fc domain comprises K370E/K409W mutations and the second Fc domain comprises E357N/D399V/F405T mutations; (2) the first Fc domain comprises E357N/D399V/F405T mutations and the second Fc domain comprises K370E/K409W mutations; (3) the first Fc domain comprises K370E/K409W mutations and the second Fc domain comprises E3571/S364T/D399V/F405T mutations; (4) the first Fc domain comprises E3571/S364T/D399V/F405T mutations and the second Fc domain comprises K370E/K409W mutations; (5) the first Fc domain comprises K370M/K409W mutations and the second Fc domain comprises E357M/S364W/D399V/F405T mutations; (6) the first Fc domain comprises E357M/S364W/D399V/F405T mutations and the second Fc domain comprises K370M/K409W mutations; (7) the first Fc domain comprises K370D/K409W mutations and the second Fc domain comprises E357M/D399V/F405T mutations; (8) the first Fc domain comprises E357M/D399V/F405T mutations and the second Fc domain comprises K370D/K409W mutations; (9) the first Fc domain comprises E357D/S364W/K370E mutations and the second Fc domain comprises E357N/K370R mutations; (10) the first Fc domain comprises E357N/K370R mutations and the second Fc domain comprises E357D/S364W/K370E mutations; (11) the first Fc domain comprises E357A/S364Y/K370E mutations and the second Fc domain comprises E357N/K370H mutations; (12) the first Fc domain comprises E357N/K370H mutations and the second Fc domain comprises E357A/S364Y/K370E mutations; (13) the first Fc domain comprises E357G/S364W/K370E mutations and the second Fc domain comprises an E357N mutation; (14) the first Fc domain comprises an E357N mutation and the second Fc domain comprises E357G/S364W/DK70E mutations; (15) the first Fc domain comprises E357N/S364W/K370E mutations and the second Fc domain comprises an E357N mutation; or (16) the first Fc domain comprises an E357N mutation and the second Fc domain comprises E357N/S364W/DK70E mutations.
32. The antibody, or antigen-binding fragment thereof, of any one of paragraphs 8-31, wherein the first and/or second Fc domain are independently engineered to have improved pharmacokinetics (PK).
33. The antibody, or antigen-binding fragment thereof, of paragraph 32, wherein the first and/or second Fe domains comprise amino acid substitutions M428L and/or N434S.
34. The antibody, or antigen-binding fragment thereof, of paragraph 10, wherein the anti-tumor antibody is an anti-CD33 antibody.
35. The antibody, or antigen-binding fragment thereof, of paragraph 34, wherein the anti-CD33 antibody comprises the six CDRs of gemtuzumab.
36. The antibody, or antigen-binding fragment thereof, of paragraph 35, wherein the anti-CD33 antibody comprises the VH of gemtuzumab.
37. The antibody, or antigen-binding fragment thereof, of paragraph 35 or 36, wherein the anti-CD33 antibody comprises the VL of gemtuzumab.
38. The antibody, or antigen-binding fragment thereof, of paragraph 34, wherein the anti-CD33 antibody comprises the VH comprising the amino acid sequence of SEQ ID NO: 1151.
39. The antibody, or antigen-binding fragment thereof, of paragraph 34 or 38, wherein the anti-CD33 antibody comprises the VL comprising the amino acid sequence of SEQ ID NO:1152.
40. The antibody, or antigen-binding fragment thereof, of any one of paragraphs 1-39, wherein the antibody is selected from an IgG antibody, an IgM antibody, an IgA antibody, an IgD antibody, and an IgE antibody.
41. The antibody, or antigen-binding fragment thereof, of paragraph 40, wherein the antibody is an IgG antibody.
42. The antibody, or antigen-binding fragment thereof, of paragraph 41, wherein the antibody is a variant of an IgG antibody selected from an IgG1 antibody, an IgG2 antibody, an IgG3 antibody or an IgG4 antibody.
43. The antibody, or antigen-binding fragment thereof, of paragraph 42, wherein the antibody comprises a modified Fc domain derived from an IgG1 molecule.
44. The antibody, or antigen-binding fragment thereof, of paragraph 43, wherein the modified Fc domain derived from the IgG1 molecule comprises an amino acid modification at any one of the positions selected from the group consisting of E216, R217, K218, C219, C220, C226, C229, P230, E233, L234, L235, G236, G237, P238, S239, V240, F241, K246, L251, T260, D265, V266, H268, W277, N297, E318, K322, P329, A330, P331, Q347, N348, T350, L351, K360, T366, N390, K392, T394, D399, S400, F405, Y407, K409, T411 or a combination such amino acid modifications.
45. The antibody, or antigen-binding fragment thereof, of paragraph 43, wherein the modified Fc domain derived from IgG1 comprises an amino acid modification selected from the group consisting of C220S, C226S, C229S, P230S, E233P, L234A, L234F, L234V, L235A, L235E, L235V, G236E, G237A, P238S, D265S, D265A, H268Q, W277T, N297G, N297Q, N297D, N297A, E318A, K322A, P329G, P329A, A330S, P331S, Q347R, Q347E, Q347K, T350V, L351Y, K360D, K360E, T366A, T366I, T366L, T366M, T366V, N390R, N390K, N390D, K392V, K392M, K392R, K392L, K392F, K392E, T394W, D399R, D399W, D399K, S400E, S400D, S400R, S400K, F405A, F405I, F405M, F405T, F405S, F405V, F405W, Y407A, Y407I, Y407L, Y407V, K409F, K409I, K409S, K409W, T411N, T411R, T411Q, T411K, T411D, T411E, T411W, ΔE216-E222, K246R/L251E/T260R, InR234/235, InV235/236, InR236/237, InR237/238, InV238/239, InN238/239, InL238/239, InE238/239, InG238/239, InS239/240, InG240/241, InE240/241, InG240/241, InL238/239/P238Q, InE238/239/N348A, InS239/240/V266A, and InR237/238/G236A or a combination thereof.
46. The antibody, or antigen-binding fragment thereof, of paragraph 45, wherein the modified Fc domain derived from IgG1 comprises L234F/L235E/P331S mutations.
47. The antibody, or antigen-binding fragment thereof, of paragraph 42, wherein the antibody comprises a modified Fc domain derived from an IgG2 molecule.
48. The antibody, or antigen-binding fragment thereof, of paragraph 47, wherein the modified Fc domain derived from the IgG2 molecule comprises an amino acid modification at any one of the positions selected from the group consisting of V234, G237, P238, H268, V309, A330, and P331 or a combination thereof.
49. The antibody, or antigen-binding fragment thereof, of paragraph 48, wherein the modified Fc domain derived from the IgG2 molecule comprises an amino acid modification at any one of the positions selected from the group consisting of V234A, G237A, P238S, H268Q, H268A, V309L, A330S, P331S, or a combination thereof.
50. The antibody, or antigen-binding fragment thereof, of paragraph 42, wherein the antibody comprises a modified Fc domain derived from an IgG4 molecule.
51. The antibody, or antigen-binding fragment thereof, of paragraph 50, wherein the modified Fc domain derived from the IgG4 molecule comprises an amino acid modification at any one of the positions selected from the group consisting of S228, L235, L236, G237, E318, and N297 or a combination thereof.
52. The antibody, or antigen-binding fragment thereof, of paragraph 51, wherein the modified Fc domain derived from the IgG4 molecule comprises an amino acid modification at any one of the positions selected from the group consisting of S228P, L235A, L235E, L236E, G237A, E318A, and N297Q or a combination thereof.
53. A recombinant polynucleotide encoding an anti-CD3 antibody, or antigen-binding fragment thereof, of any one of paragraphs 1-52.
54. An expression vector comprising the recombinant polynucleotide of paragraph 53.
55. A first recombinant polynucleotide encoding the heavy chain of an anti-CD3 antibody, or antigen-binding fragment thereof, of any one of paragraphs 1-52 and a second recombinant polynucleotide encoding the light chain of an anti-CD3 antibody, or antigen-binding fragment thereof, of any one of paragraphs 1-52.
56. A first expression vector comprising the first recombinant polynucleotide of paragraph 55 and a second expression vector comprising the second recombinant polynucleotide of paragraph 55.
57. An isolated host cell comprising the recombinant polynucleotide of paragraph 53, the expression vector of paragraph 54, the first and second recombinant polynucleotides of paragraph 55 or the first and second expression vectors of paragraph 56.
58. A method of producing the antibody, or antigen-binding fragment thereof, or any one of paragraphs 1-52, the method comprising culturing the host cell of paragraph 57 under conditions suitable for expressing the antibody or fragment.
59. A composition comprising the antibody, or antigen-binding fragment thereof, of any one of paragraphs 1-52 and a physiologically acceptable carrier.
60. A method of treating or preventing cancer in a subject in need thereof, the method comprising administering the antibody, or antigen-binding fragment thereof, of any one of paragraphs 1-52 or the composition of paragraph 59 to the subject in need thereof.
61. A method of maintaining cancer remission in a subject, the method comprising administering the antibody, or antigen-binding fragment thereof, of any one of paragraphs 1-52 or the composition of paragraph 59 to the subject in need thereof.
62. A method of targeting a T-cell to a tumor cell, wherein the method comprises contacting the T-cell with the antibody, or antigen-binding fragment thereof, any one of paragraphs 8-52 or the composition of paragraph 59.
63. The method of any one of paragraphs 60-62, wherein the anti-CD3 antibody is an anti-CD3 bispecific antibody, wherein the bispecific antibody further comprises an anti-tumor antibody.
64. A method of treating or preventing an autoimmune disease or disorder in a subject in need thereof, the method comprising administering the antibody, or antigen-binding fragment thereof, of any one of paragraphs 1-52 or the composition of paragraph 59 to the subject in need thereof
65. The method of paragraph 64, wherein the autoimmune disease or disorder is graft-versus-host disease or type I diabetes.

### EXAMPLES

### Example 1

### Generation of Anti-CD3 Hybridomas

### Immunizations

Two cohorts of AlivaMab® mice (AMM-XKL and AMM-LO) were immunized with recombinant human and cynomolgus CD3 proteins, followed by a boost with human T cells. Titers were checked by flow cytometry against Jurkat wild type cells and Jurkat cells with TCRβ knockout (which prevents the entire TCR complex including CD3 from reaching the cell surface) as a negative control.

### Fusion and Screening

Spleen and lymph node cells were isolated from AlivaMab® Mice and enriched for IgG-antibody expressing cells, then electro-fused with cells from myeloma cell line (ID), seeded into 384 well plates and in HAT medium to select for hybridomas. Primary screening was performed by flow cytometry against Jurkat wild-type cells and Jurkat cells with TCR knockout (RT3-T3.5 cells) as a negative control using a fluorchrome-conjugated anti-mouse IgG Fc to detect binding of the chimeric AlivaMab® antibodies to the cells. Confirmatory screens were performed by ELISA against the recombinant human and cynomolgus CD3 proteins as well as by flow cytometry against Jurkat cells and human Pan-T cells. Bio-Layer Interferometry (Sartorius Octet) assays were performed to assess the kinetics of binding of the hits to recombinant human CD3 protein. Selected hits (Table 4, infra) were subcloned, scaled up for purification and sequencing by next generation sequencing (NGS).

TABLE 4
Flow Cytometry Analysis of Unpurified Monoclonal Antibodies

Flow Cytometry Binding

| | Jurkat | RT3-T3.5 | human Pan |
|---|---|---|---|
| | WT | (TCR-KO) | T cells |
| Hybridoma | (gMFI) | (gMFI) | (gMFI) |
| CD3-Hyb1 | 105241 | 5534 | 529735 |
| CD3-Hyb2 | 101830 | 4401 | 454617 |
| CD3-Hyb3 | 100603 | 4551 | 540508 |
| CD3-Hyb4 | 99450 | 5109 | 508188 |
| CD3-Hyb5 | 99437 | 5048 | 480404 |
| CD3-Hyb6 | 93216 | 4710 | 530445 |
| CD3-Hyb7 | 91821 | 4993 | 585070 |
| CD3-Hyb8 | 87587 | 4894 | 549489 |
| CD3-Hyb9 | 82868 | 3922 | 548634 |
| CD3-Hyb10 | 81293 | 4370 | 341704 |
| CD3-Hyb11 | 80112 | 4347 | 350579 |
| CD3-Hyb12 | 79579 | 4086 | 478964 |
| CD3-Hyb13 | 76514 | 3969 | 447871 |
| CD3-Hyb14 | 71893 | 4097 | 184589 |
| CD3-Hyb15 | 71578 | 4065 | 231850 |
| CD3-Hyb16 | 55653 | 3538 | 227843 |
| CD3-Hyb17 | 54573 | 2930 | 157136 |
| CD3-Hyb18 | 51691 | 3430 | 170643 |
| CD3-Hyb19 | 46134 | 2531 | 234643 |
| CD3-Hyb20 | 44971 | 3010 | 198988 |
| CD3-Hyb21 | 39795 | 3048 | 114707 |
| CD3-Hyb22 | 36651 | 2871 | 172601 |
| CD3-Hyb23 | 34294 | 2823 | 76366 |
| CD3-Hyb24 | 25319 | 2763 | 69652 |
| CD3-Hyb25 | 23088 | 2101 | 91001 |
| OKT3 | 72347 | 4223 | 300310 |
| SP34 | 113713 | 7165 | 332761 |
| UCHT1 | 102228 | 5408 | 358900 |
| Isotype Control | 1708 | 1818 | 4373 |

### Example 2

### Generation of Antibody Sequences from Hybridomas and Ngs Clusters

### Hybridoma Sequencing

Hybridoma cells were lysed, and RNA was isolated. First strand cDNA synthesis was performed using reverse transcriptase primed with oligo dT. Second strand synthesis was performed using a template switching oligonucleotide. Ig-encoding fragments were then generated by polymerase chain reaction using a universal upstream primer and gene-specific Ig downstream primers. Barcodes and Illumina adaptor sequences were added, and samples were sequenced using a MiSeq to generate 2×300 bp paired-end reads. Sequences were sorted by barcode and analyzed to generate nucleotide sequences from which amino acid sequences were derived.

Immune Repertoire Sequencing—Identification of Antibodies that Specifically Bind CD3

Secondary lymphoid organs were harvested from immunized mice (spleen and lymph nodes). After grinding the tissue and extracting its RNA, the samples were sequenced via paired-single cell NGS (10×-sequences) from Azenta Life Sciences. Sequences were delivered as fasta files along with a csv (comma-separated values) denoting DNA barcodes for each sequence. PipeBio Bioinformatics Suite (PipeBio ApS, Horsens, Denmark) was used to pair heavy and light chain sequences of the same cell via their respective shared barcode sequences. Paired sequences were pre-clustered according to their VH gene and then clustered on the abundance of their H-CDR3 sequences above a Hamming Distance identity threshold. The most common sequence within the cluster was treated as its representative. The representatives of the largest clusters were cloned, as follows.

Cloning and Expression of Sequences from Immune Repertoire

Heavy chain and light chain sequences for an antibody were separately ordered as gblocks (IDT) and then stitched into an expression plasmid vector using infusion (Takara Bio). Standard molecular biology techniques were used to create the respective heavy and light chain expression plasmids for each recombinant antibody. HEK-293 (Expi293, Thermo, Carlsbad, CA) cells were transfected with the positive clones according to manufacturer's protocol. Cultures were harvested and recombinant antibodies were purified using Protein A-based chromatography methods. Purified recombinant antibodies were tested for activity against controls using the same methods for bispecific antibodies.

### Example 3

### Purification of Monoclonal Antibodies from Hybridoma Supernatants and Characterization of Binding

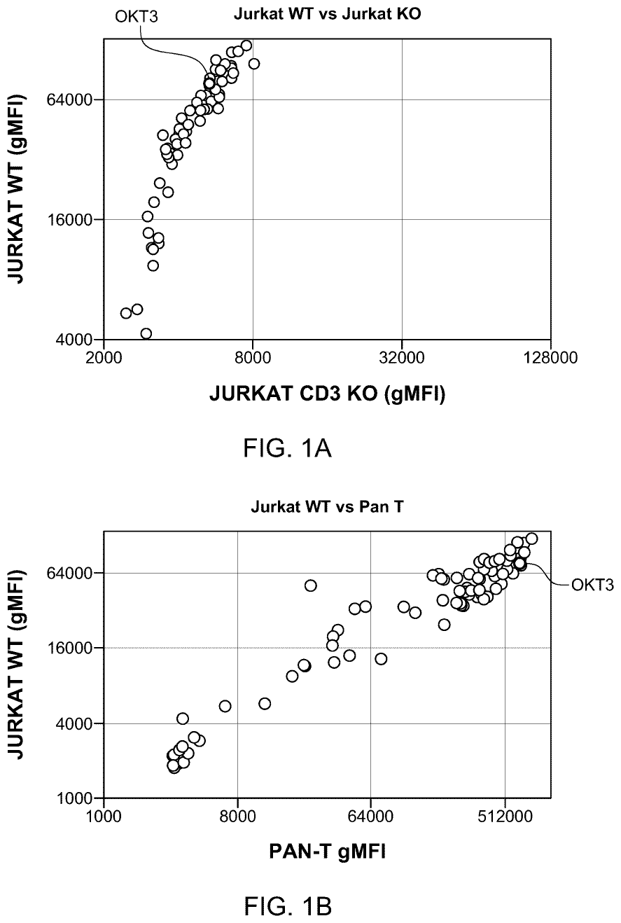

A panel of antibodies comprising unique VH and VL sequences was purified by protein A affinity chromatography. Hybridoma supernatants were incubated with protein A beads, the beads were washed, and antibodies were eluted with acidic pH buffer and neutralized. Samples were exchanged into phosphate buffered saline (PBS) and analyzed by HPLC-SEC (Table 5, infra). Samples were evaluated by flow cytometry analysis against Jurkat and human Pan-T cells (FIG. 1B) as well as by Octet against recombinant human CD3 proteins to evaluate apparent affinities and binding off-rates (Table 6, infra). Antibodies with diverse sequences and showing binding to recombinant CD3 protein, on CD3⁺ cell lines and on primary cells, and with a fast off-rate were selected for reformatting into a bispecific format.

TABLE 5
Expression and Purification Characteristics
of Antibodies from Hybridoma

| | Purified | | | |
|---|---|---|---|---|
| | Hybridoma | SEC-HPLC | | |
| Source | mAbs Yield | % | % | % |
| Antibody ID | (mg/L) | HMW | Main | LMW |
| ABL-CD3-1 | 8 | 3.6 | 94.8 | 1.6 |
| ABL-CD3-2 | 14 | 0.4 | 98.4 | 1.1 |
| ABL-CD3-3 | 30 | 1.2 | 97.5 | 1.3 |
| ABL-CD3-4 | 38 | 4.4 | 93.8 | 1.8 |
| ABL-CD3-5 | 41 | 1.9 | 96.8 | 1.3 |
| ABL-CD3-6 | 29 | 1.2 | 97.2 | 1.5 |
| ABL-CD3-7 | 17 | 3.6 | 92.0 | 4.4 |
| ABL-CD3-8 | 16 | 2.4 | 95.5 | 2.0 |
| ABL-CD3-9 | 18 | 1.6 | 96.3 | 2.1 |
| ABL-CD3-10 | 10 | ND | ND | ND |
| ABL-CD3-12 | 93 | 2.1 | 97.6 | 0.4 |
| ABL-CD3-13 | 18 | 2.4 | 96.5 | 1.1 |
| ABL-CD3-14 | 67 | 1.1 | 96.8 | 2.1 |
| ABL-CD3-15 | 12 | 1.2 | 90.2 | 8.7 |
| ABL-CD3-16 | 23 | 3.0 | 95.2 | 1.9 |
| ABL-CD3-17 | 21 | 0.8 | 97.7 | 1.5 |
| ABL-CD3-18 | 34 | 3.6 | 92.7 | 3.6 |
| ABL-CD3-19 | 93 | 1.2 | 96.4 | 2.4 |
| ABL-CD3-20 | 114 | 0.4 | 98.6 | 1.0 |
| ABL-CD3-21 | 23 | ND | ND | ND |
| ABL-CD3-22 | 73 | 1.6 | 95.7 | 2.6 |
| ABL-CD3-24 | 46 | 2.0 | 94.5 | 3.5 |
| ABL-CD3-25 | 69 | 1.5 | 96.2 | 2.3 |
| ABL-CD3-26 | 72 | 1.3 | 96.4 | 2.3 |
| ABL-CD3-27 | 64 | 3.5 | 93.8 | 2.8 |
| ABL-CD3-28 | 43 | 1.1 | 94.6 | 4.3 |
| ABL-CD3-29 | 48 | 0.1 | 71.1 | 28.8 |
| ABL-CD3-31 | 23 | 0.0 | 95.2 | 4.8 |
| ABL-CD3-32 | 52 | 2.1 | 95.9 | 2.0 |
| ABL-CD3-33 | 42 | 2.2 | 95.3 | 2.5 |
| ABL-CD3-34 | 33 | 2.7 | 94.4 | 2.9 |
| ABL-CD3-35 | 18 | 3.4 | 93.7 | 2.9 |
| ABL-CD3-36 | 102 | 0.2 | 94.9 | 4.9 |
| ABL-CD3-37 | 38 | 0.8 | 91.5 | 7.7 |
| ABL-CD3-38 | 24 | 3.8 | 87.2 | 9.0 |
| ABL-CD3-39 | 111 | 1.5 | 96.0 | 2.5 |
| ABL-CD3-114 | 32 | ND | ND | ND |
| ABL-CD3-41 | 17 | 3.4 | 93.8 | 2.8 |
| ABL-CD3-42 | 62 | 1.5 | 95.6 | 2.9 |
| ABL-CD3-43 | 55 | 2.0 | 94.4 | 3.6 |
| ABL-CD3-44 | 43 | 4.1 | 93.4 | 2.6 |
| ABL-CD3-45 | 89 | 2.2 | 95.5 | 2.3 |
| ABL-CD3-46 | 38 | 2.6 | 92.6 | 4.8 |
| ABL-CD3-47 | 32 | 2.6 | 93.7 | 3.6 |
| ABL-CD3-48 | 38 | 0.9 | 96.6 | 2.5 |
| ABL-CD3-49 | 119 | 8.4 | 90.3 | 1.3 |
| ABL-CD3-50 | 55 | 8.5 | 89.1 | 2.4 |
| ABL-CD3-51 | 38 | 3.0 | 95.0 | 2.0 |
| ABL-CD3-52 | 16 | 3.5 | 90.8 | 5.7 |
| ABL-CD3-53 | 12 | 0.2 | 94.9 | 5.0 |
| ABL-CD3-54 | 53 | 1.7 | 93.1 | 5.2 |
| ABL-CD3-55 | 34 | 7.6 | 88.3 | 4.2 |
| ABL-CD3-56 | 20 | 0.9 | 92.3 | 6.8 |
| ABL-CD3-57 | 26 | 2.1 | 93.8 | 4.1 |
| ABL-CD3-58 | 11 | 2.9 | 91.2 | 5.9 |
| ABL-CD3-59 | 24 | 1.1 | 93.9 | 5.1 |
| ABL-CD3-60 | 28 | 0.8 | 94.6 | 4.6 |
| ABL-CD3-61 | 12 | 2.0 | 93.2 | 4.8 |
| ABL-CD3-62 | 27 | 2.5 | 92.2 | 5.2 |
| ABL-CD3-63 | 27 | 0.7 | 93.2 | 6.1 |
| ABL-CD3-64 | 34 | 2.8 | 93.8 | 3.4 |
| ABL-CD3-65 | 54 | 3.3 | 92.0 | 4.7 |
| ABL-CD3-66 | 26 | 1.4 | 92.7 | 5.9 |
| ABL-CD3-67 | 42 | 1.4 | 94.9 | 3.7 |
| ABL-CD3-68 | 1 | ND | ND | ND |
| ABL-CD3-69 | 29 | 2.3 | 92.7 | 5.0 |
| ABL-CD3-70 | 34 | 2.3 | 92.6 | 5.1 |
| ABL-CD3-71 | 54 | 2.5 | 92.1 | 5.4 |
| ABL-CD3-72 | 20 | 1.5 | 93.1 | 5.4 |
| ABL-CD3-73 | 58 | 0.9 | 95.1 | 4.0 |
| ABL-CD3-74 | 20 | 2.0 | 93.3 | 4.7 |
| ABL-CD3-75 | 9 | 1.2 | 91.9 | 6.9 |
| ABL-CD3-76 | 36 | 3.5 | 90.2 | 5.3 |
| ABL-CD3-77 | 22 | 4.7 | 91.3 | 4.0 |
| ABL-CD3-78 | 71 | 0.6 | 94.9 | 4.5 |
| ABL-CD3-79 | 71 | ND | ND | ND |
| ABL-CD3-80 | 3 | 6.2 | 90.1 | 3.7 |
| ABL-CD3-81 | 6 | 3.6 | 93.1 | 3.3 |
| ABL-CD3-82 | 34 | ND | ND | ND |
| ABL-CD3-83 | 49 | 19.8 | 67.2 | 13.0 |
| ABL-CD3-84 | 2 | ND | ND | ND |
| ABL-CD3-85 | 34 | 2.0 | 92.7 | 5.4 |
| ABL-CD3-86 | 34 | 2.9 | 92.8 | 4.3 |
| ABL-CD3-87 | 20 | 1.2 | 94.1 | 4.6 |

*ND—not determined.

TABLE 6
Binding Kinetics of Purified mAbs to Recombinant CD3 Proteins

| | hCD3 E/D, 200-50 nM, FAB2G Sensor, 5 ug/mL mAb | | | | | hCD3 E/G, 200-50 nM, FAB2G Sensor, 5 ug/mL mAb | | | | |
|---|---|---|---|---|---|---|---|---|---|---|
| Hybridoma ID | Response | KD (M) | kdis(1/s) | kon(1/Ms) | Full R\hat{ }2 | Response | KD (M) | kdis(1/s) | kon(1/Ms) | Full R\hat{ }2 |
| ABL-CD3-1 | 0.31 | 1.08E−07 | 1.57E−02 | 1.45E+05 | 1.00 | 0.07 | 1.21E−06 | 1.71E−01 | 1.42E+05 | 0.94 |
| ABL-CD3-2 | 0.09 | 4.11E−10 | 1.56E−03 | 3.80E+06 | 0.10 | 0.05 | <1.0E−12 | <1.0E−07 | 2.30E+06 | 0.02 |
| ABL-CD3-3 | 1.03 | 1.04E−08 | 1.20E−03 | 1.16E+05 | 0.99 | 0.96 | 1.24E−08 | 1.43E−03 | 1.15E+05 | 0.99 |
| ABL-CD3-4 | 0.32 | 9.45E−08 | 3.02E−02 | 3.20E+05 | 0.99 | 0.36 | 9.19E−08 | 2.21E−02 | 2.41E+05 | 0.99 |
| ABL-CD3-5 | 0.36 | 8.81E−08 | 3.21E−02 | 3.65E+05 | 0.99 | 0.27 | 1.06E−07 | 4.49E−02 | 4.23E+05 | 0.99 |
| ABL-CD3-6 | 0.30 | 6.97E−08 | 3.33E−02 | 4.77E+05 | 1.00 | 0.17 | 1.38E−07 | 7.79E−02 | 5.66E+05 | 1.00 |
| ABL-CD3-7 | 0.86 | 4.14E−09 | 7.86E−04 | 1.90E+05 | 0.99 | 0.81 | 8.42E−09 | 1.42E−03 | 1.69E+05 | 0.99 |
| ABL-CD3-8 | 0.11 | 2.83E−07 | 2.12E−01 | 7.48E+05 | 0.99 | 0.02 | 2.97E−10 | 1.49E+00 | 5.02E+09 | 0.85 |
| ABL-CD3-9 | 0.34 | 7.08E−08 | 3.93E−02 | 5.55E+05 | 0.99 | 0.10 | 1.10E−07 | 1.14E−01 | 1.04E+06 | 0.84 |
| ABL-CD3-10 | 0.52 | 2.47E−09 | 4.60E−04 | 1.86E+05 | 0.99 | 0.46 | 2.41E−09 | 3.90E−04 | 1.62E+05 | 0.99 |
| ABL-CD3-12 | 1.21 | 1.06E−08 | 1.44E−03 | 1.36E+05 | 0.99 | 1.14 | 1.05E−08 | 1.24E−03 | 1.18E+05 | 0.99 |
| ABL-CD3-13 | 0.08 | 2.34E−08 | 7.28E−04 | 3.12E+04 | 0.96 | 0.01 | <1.0E−12 | <1.0E−07 | 2.67E+04 | 0.34 |
| ABL-CD3-14 | 0.17 | 1.28E−08 | 1.52E−03 | 1.19E+05 | 0.97 | 0.08 | 9.66E−09 | 2.87E−03 | 2.98E+05 | 0.94 |
| ABL-CD3-15 | 0.93 | 1.62E−09 | 2.76E−04 | 1.71E+05 | 0.99 | 0.83 | 6.54E−09 | 1.13E−03 | 1.73E+05 | 1.00 |
| ABL-CD3-16 | 0.19 | 2.95E−08 | 1.53E−03 | 5.19E+04 | 0.99 | 0.01 | <1.0E−12 | <1.0E−07 | 3.62E+04 | 0.63 |
| ABL-CD3-17 | 0.22 | 1.03E−07 | 3.18E−02 | 3.09E+05 | 0.97 | 0.09 | 2.10E−07 | 1.32E−01 | 6.27E+05 | 0.91 |
| ABL-CD3-18 | 0.07 | <1.0E−12 | <1.0E−07 | 2.91E+04 | 0.60 | 0.04 | <1.0E−12 | <1.0E−07 | 2.70E+04 | 0.49 |
| ABL-CD3-19 | 0.60 | 2.33E−08 | 1.91E−03 | 8.17E+04 | 1.00 | 0.40 | 6.29E−08 | 6.65E−03 | 1.06E+05 | 1.00 |
| ABL-CD3-20 | 0.51 | 1.05E−08 | 2.29E−03 | 2.19E+05 | 0.99 | 0.45 | 1.83E−08 | 3.79E−03 | 2.07E+05 | 0.99 |
| ABL-CD3-21 | 0.27 | 9.53E−08 | 3.60E−02 | 3.78E+05 | 0.98 | 0.11 | 1.30E−07 | 9.94E−02 | 7.66E+05 | 0.79 |
| ABL-CD3-22 | 0.08 | 4.92E−08 | 5.13E−03 | 1.04E+05 | 0.96 | 0.02 | 1.44E−08 | 6.39E−03 | 4.44E+05 | 0.85 |
| ABL-CD3-24 | 0.93 | 5.78E−09 | 1.01E−03 | 1.75E+05 | 0.99 | 0.89 | 8.48E−09 | 1.34E−03 | 1.58E+05 | 0.99 |
| ABL-CD3-25 | 1.32 | 8.56E−09 | 9.33E−04 | 1.09E+05 | 0.99 | 1.27 | 1.17E−08 | 1.13E−03 | 9.66E+04 | 1.00 |
| ABL-CD3-26 | 1.03 | 6.66E−09 | 1.04E−03 | 1.56E+05 | 0.99 | 1.01 | 9.31E−09 | 1.23E−03 | 1.32E+05 | 0.99 |
| ABL-CD3-27 | 0.39 | 2.31E−09 | 5.28E−04 | 2.28E+05 | 0.96 | 0.03 | 3.65E−09 | 1.56E−03 | 4.28E+05 | 0.64 |
| ABL-CD3-28 | 0.77 | 7.88E−09 | 1.33E−03 | 1.68E+05 | 0.99 | 0.77 | 5.60E−09 | 7.98E−04 | 1.43E+05 | 0.99 |
| ABL-CD3-29 | 0.46 | 2.63E−09 | 8.16E−04 | 3.10E+05 | 0.94 | 0.44 | 2.50E−09 | 8.52E−04 | 3.41E+05 | 0.94 |
| ABL-CD3-31 | 0.32 | 6.66E−09 | 1.49E−03 | 2.23E+05 | 0.94 | 0.30 | 5.64E−09 | 1.73E−03 | 3.06E+05 | 0.92 |
| ABL-CD3-32 | 0.36 | 3.89E−08 | 1.52E−02 | 3.90E+05 | 0.95 | 0.40 | 3.16E−08 | 9.62E−03 | 3.05E+05 | 0.96 |
| ABL-CD3-33 | 0.33 | 5.62E−08 | 2.06E−02 | 3.66E+05 | 0.96 | 0.34 | 6.00E−08 | 1.55E−02 | 2.59E+05 | 0.96 |
| ABL-CD3-34 | 0.93 | 9.01E−09 | 2.11E−03 | 2.34E+05 | 0.98 | 0.89 | 9.82E−09 | 1.96E−03 | 1.99E+05 | 0.98 |
| ABL-CD3-35 | 0.36 | 5.47E−08 | 2.71E−02 | 4.95E+05 | 0.97 | 0.12 | 1.42E−08 | 1.44E−02 | 1.02E+06 | 0.84 |
| ABL-CD3-36 | 0.49 | 2.50E−08 | 1.01E−03 | 4.04E+04 | 1.00 | 0.02 | 4.18E−09 | 7.29E−03 | 1.74E+06 | 0.76 |
| ABL-CD3-37 | 0.09 | 1.78E−08 | 4.04E−03 | 2.27E+05 | 0.92 | 0.05 | 1.43E−09 | 2.69E−03 | 1.89E+06 | 0.59 |
| ABL-CD3-38 | 0.73 | 1.41E−09 | 3.50E−04 | 2.48E+05 | 0.97 | 0.73 | 1.36E−09 | 3.31E−04 | 2.43E+05 | 0.98 |
| ABL-CD3-39 | 0.41 | 8.32E−09 | 3.07E−04 | 3.69E+04 | 1.00 | 0.00 | <1.0E−12 | <1.0E−07 | 5.00E+04 | 0.00 |
| ABL-CD3-40 | 0.05 | <1.0E−12 | <1.0E−07 | 1.77E+04 | 0.60 | 0.06 | <1.0E−12 | <1.0E−07 | 2.28E+04 | 0.49 |
| ABL-CD3-41 | 0.34 | 8.50E−08 | 3.95E−02 | 4.64E+05 | 1.00 | 0.10 | 2.95E−07 | 2.50E−01 | 8.46E+05 | 0.99 |
| ABL-CD3-42 | 0.10 | 2.55E−07 | 9.95E−03 | 3.90E+04 | 0.99 | 0.00 | <1.0E−12 | 3.85E−01 | 4.44E+89 | 0.37 |
| ABL-CD3-43 | 0.62 | 6.20E−09 | 7.54E−04 | 1.22E+05 | 0.99 | 0.62 | 6.94E−09 | 1.04E−03 | 1.50E+05 | 0.98 |
| ABL-CD3-44 | 0.03 | 3.56E−07 | 1.84E−04 | 5.17E+02 | 0.98 | 0.00 | <1.0E−12 | 7.44E−01 | 1.27E+134 | 0.90 |
| ABL-CD3-45 | 0.20 | 1.37E−08 | 4.75E−04 | 3.46E+04 | 1.00 | 0.03 | 1.58E−08 | 3.75E−03 | 2.37E+05 | 0.84 |
| ABL-CD3-46 | 1.02 | 9.48E−09 | 1.71E−03 | 1.80E+05 | 0.99 | 0.88 | 1.63E−08 | 3.33E−03 | 2.04E+05 | 0.98 |
| ABL-CD3-47 | 0.62 | 1.69E−08 | 2.91E−03 | 1.72E+05 | 0.99 | 0.43 | 3.62E−08 | 7.85E−03 | 2.17E+05 | 0.99 |
| ABL-CD3-48 | 1.17 | 1.15E−08 | 1.14E−03 | 9.93E+04 | 0.99 | 1.03 | 1.79E−08 | 1.79E−03 | 1.00E+05 | 0.99 |
| ABL-CD3-49 | 0.93 | 6.52E−09 | 1.31E−03 | 2.00E+05 | 0.99 | 0.82 | 1.14E−08 | 2.53E−03 | 2.23E+05 | 0.98 |
| ABL-CD3-50 | 0.57 | 2.10E−08 | 4.70E−03 | 2.24E+05 | 0.99 | 0.60 | 1.26E−08 | 2.47E−03 | 1.96E+05 | 0.99 |
| ABL-CD3-51 | 0.21 | 7.71E−08 | 1.88E−02 | 2.44E+05 | 0.98 | 0.06 | 2.33E−07 | 1.63E−01 | 6.99E+05 | 0.98 |
| ABL-CD3-52 | 0.35 | 5.40E−08 | 2.02E−02 | 3.73E+05 | 0.98 | 0.14 | 2.33E−07 | 1.15E−01 | 4.95E+05 | 0.98 |
| ABL-CD3-53 | 0.24 | 1.46E−07 | 1.99E−02 | 1.36E+05 | 0.99 | 0.40 | 8.87E−08 | 7.14E−03 | 8.05E+04 | 0.99 |
| ABL-CD3-54 | 0.87 | 1.76E−08 | 2.67E−03 | 1.52E+05 | 0.99 | 0.87 | 1.93E−08 | 2.58E−03 | 1.34E+05 | 0.99 |
| ABL-CD3-55 | 0.13 | 1.83E−07 | 6.90E−02 | 3.76E+05 | 0.99 | 0.08 | 1.41E−07 | 8.94E−02 | 6.36E+05 | 0.99 |
| ABL-CD3-56 | 1.24 | 3.96E−09 | 5.22E−04 | 1.32E+05 | 0.99 | 1.30 | 9.40E−10 | 1.02E−04 | 1.09E+05 | 0.99 |
| ABL-CD3-57 | 0.49 | 4.29E−09 | 2.77E−04 | 6.47E+04 | 0.98 | 0.54 | 3.97E−09 | 2.60E−04 | 6.54E+04 | 0.99 |
| ABL-CD3-58 | 0.57 | 4.41E−08 | 2.65E−03 | 6.01E+04 | 1.00 | 0.28 | 4.52E−08 | 7.05E−03 | 1.56E+05 | 0.94 |
| ABL-CD3-59 | 0.81 | 1.27E−08 | 1.05E−03 | 8.31E+04 | 1.00 | 0.58 | 4.33E−08 | 4.87E−03 | 1.13E+05 | 0.99 |
| ABL-CD3-60 | 0.10 | 2.31E−07 | 9.62E−02 | 4.17E+05 | 0.99 | 0.07 | 9.07E−08 | 5.83E−02 | 6.42E+05 | 0.98 |
| ABL-CD3-61 | 0.26 | 1.17E−07 | 2.18E−02 | 1.87E+05 | 0.98 | 0.07 | 6.92E−08 | 4.02E−02 | 5.81E+05 | 0.95 |
| ABL-CD3-62 | 0.56 | 3.40E−08 | 7.65E−03 | 2.25E+05 | 0.98 | 0.69 | 2.38E−08 | 3.91E−03 | 1.64E+05 | 0.98 |
| ABL-CD3-63 | 0.89 | 1.15E−08 | 1.45E−03 | 1.27E+05 | 0.99 | 0.34 | 9.04E−08 | 1.82E−02 | 2.02E+05 | 0.98 |
| ABL-CD3-64 | 0.68 | 3.16E−08 | 5.40E−03 | 1.71E+05 | 0.99 | 0.46 | 7.19E−08 | 1.30E−02 | 1.80E+05 | 0.99 |
| ABL-CD3-65 | 0.01 | 2.27E−08 | 6.43E−03 | 2.83E+05 | 0.59 | 0.01 | 1.87E−08 | 1.74E−03 | 9.32E+04 | 0.88 |
| ABL-CD3-66 | 0.41 | 7.82E−08 | 2.66E−02 | 3.41E+05 | 0.99 | 0.09 | 9.28E−08 | 8.56E−02 | 9.23E+05 | 0.98 |
| ABL-CD3-67 | 0.58 | 5.33E−08 | 4.85E−03 | 9.08E+04 | 0.99 | 0.26 | 1.09E−07 | 1.78E−02 | 1.63E+05 | 0.97 |
| ABL-CD3-68 | 0.30 | 4.20E−09 | 9.73E−04 | 2.32E+05 | 0.98 | 0.28 | 6.43E−09 | 1.57E−03 | 2.44E+05 | 0.98 |
| ABL-CD3-69 | 0.87 | 8.14E−09 | 8.51E−04 | 1.05E+05 | 0.99 | 0.88 | 3.73E−09 | 3.21E−04 | 8.61E+04 | 0.99 |
| ABL-CD3-70 | 0.26 | 4.71E−08 | 1.01E−02 | 2.14E+05 | 0.94 | 0.11 | 4.87E−09 | 3.39E−03 | 6.97E+05 | 0.85 |
| ABL-CD3-71 | 0.48 | 5.88E−08 | 1.02E−02 | 1.73E+05 | 0.99 | 0.11 | 5.50E−08 | 3.29E−02 | 5.98E+05 | 0.86 |
| ABL-CD3-72 | 0.17 | 1.09E−07 | 7.00E−02 | 6.41E+05 | 0.98 | 0.09 | 4.42E−07 | 2.11E−01 | 4.76E+05 | 0.99 |
| ABL-CD3-73 | 1.02 | 1.26E−09 | 1.00E−04 | 7.97E+04 | 0.99 | 0.99 | 6.12E−09 | 4.72E−04 | 7.70E+04 | 1.00 |
| ABL-CD3-74 | 0.24 | 5.56E−08 | 2.60E−02 | 4.67E+05 | 0.97 | 0.13 | 1.24E−07 | 6.09E−02 | 4.92E+05 | 0.99 |
| ABL-CD3-75 | 0.56 | 2.96E−08 | 7.26E−03 | 2.45E+05 | 0.98 | 0.19 | 1.59E−07 | 5.93E−02 | 3.73E+05 | 0.98 |
| ABL-CD3-76 | 0.53 | 4.82E−08 | 1.25E−02 | 2.60E+05 | 0.98 | 0.24 | 1.34E−07 | 4.61E−02 | 3.45E+05 | 0.99 |
| ABL-CD3-77 | 0.01 | <1.0E−12 | 1.35E−02 | 9.36E+12 | 0.61 | −0.01 | <1.0E−12 | <1.0E−07 | 5.00E+04 | 0.00 |
| ABL-CD3-78 | 0.56 | 6.64E−09 | 5.68E−04 | 8.56E+04 | 1.00 | 0.50 | 2.14E−08 | 1.57E−03 | 7.35E+04 | 1.00 |
| ABL-CD3-79 | 0.22 | 1.20E−07 | 4.73E−02 | 3.93E+05 | 0.99 | 0.07 | 4.12E−07 | 1.62E−01 | 3.93E+05 | 0.99 |
| ABL-CD3-80 | 0.36 | 1.25E−07 | 1.08E−02 | 8.62E+04 | 0.99 | 0.11 | 1.48E−07 | 3.60E−02 | 2.43E+05 | 0.95 |
| ABL-CD3-81 | 0.17 | 2.83E−07 | 1.07E−01 | 3.78E+05 | 1.00 | 0.04 | 9.12E−07 | 6.92E−01 | 7.59E+05 | 0.95 |
| ABL-CD3-82 | 0.02 | 1.06E−08 | 1.92E−03 | 1.82E+05 | 0.82 | 0.02 | 3.18E−09 | 1.97E−03 | 6.20E+05 | 0.81 |
| ABL-CD3-83 | 0.03 | 2.20E−08 | 8.82E−03 | 4.01E+05 | 0.89 | 0.01 | 3.76E−08 | 6.49E−03 | 1.73E+05 | 0.77 |
| ABL-CD3-84 | 0.22 | 1.43E−07 | 2.03E−02 | 1.42E+05 | 0.98 | 0.10 | 1.45E−07 | 6.14E−02 | 4.24E+05 | 0.95 |
| ABL-CD3-85 | 0.042* | 2.79E−08 | 6.89E−03 | 2.47E+05 | 0.99 | 0.64 | 2.98E−08 | 6.08E−03 | 2.04E+05 | 0.98 |
| ABL-CD3-86 | 1.10 | 3.88E−09 | 7.11E−04 | 1.84E+05 | 0.99 | 1.17 | 3.52E−09 | 5.58E−04 | 1.58E+05 | 0.99 |
| ABL-CD3-87 | 0.0358* | 6.90E−08 | 1.66E−02 | 2.40E+05 | 0.98 | 0.36 | 1.31E−07 | 2.79E−02 | 2.14E+05 | 0.99 |

*Purified mAbs were assessed by Octet against recombinant human CD3E/D proteins.

### Example 4

### Generation and Characterization of Bispecific Antibodies

### Generation of Plasmids Encoding Bispecific Constructs

Sequences derived from hybridoma candidates were selected for cloning based on FACS and Octet analysis as described in Example 3. These were reformatted as scFv in VL-VH and VH-VL orientations of the variable heavy and light chains. These were made as disulfide bond stabilized scFv by mutating VH44 and VL100 to Cys (Kabat numbering). These scFvs were cloned onto an Fc from a human IgG1 antibody and paired with a Fab derived from gemtuzumab also cloned onto an Fc from a human IgG1 antibody. Production of BiSAbs as hetero-dimers was driven by a knobs-into-holes (KiH) approach (Merchant et al., Nat Biotechnol, 16:677-81, 1998) achieved by introducing two amino acid changes in CH3 domain (S354C/T366W) for knob and four amino acid changes Y349C/T366S/L368A/Y407V for hole (according to Eu numbering). Further, the hole is mutated to eliminate protein A binding (H435R/Y436F). In this way, the knob can bind to protein A and the hole can bind to Fab-selective resins, offering multiple purification options. The knob mutations were introduced into the Fc of the anti-CD3 antibody, and the hole mutations were introduced into the Fc of gemtuzumab. Additionally, L234F/L235E/P331S (i.e. “triple mutation” or “TM”) were introduced into the Fc regions of both arms of the bispecific antibodies to reduce effector function. See, e.g., Oganesyan V, et al., Acta Crystallogr D Biol Crystallogr. 2008 June; 64(Pt 6):700-4, which is incorporated herein by reference in its entirety.

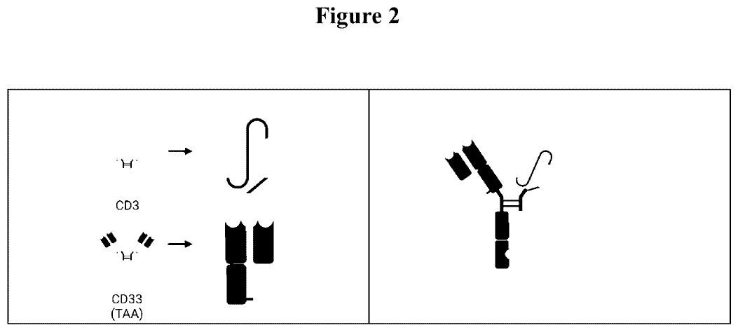

Cloning into TGEX eukaryotic expression vectors was performed with Takara Infusion HD using standard molecular biology techniques. Positive clones were confirmed by rolling circle amplification (RCA) followed by Sanger sequencing and milligram quantity DNA was made using Endo-EF maxiprep kit (Takara) to achieve low endotoxin levels. The panel of CD3 stabilized scFv-knob Fcs were then transfected along with gemtuzumab-hole HC and gemtuzumab LC in Expi293 to make KiH BisAbs (FIG. 2, Table 7, infra). Gemtuzumab HC Hole/RF/TM (SEQ ID NO: 1150):

Gemtuzumab HC Hole/RF/TM (SEQ ID NO: 1150):
EVQLVQSGAEVKKPGSSVKVSCKASGYTITDSNIHWVRQAPGQSLEWIGYIYPYNGG
TDYNQKFKNRATLTVDNPTNTAYMELSSLRSEDTAFYYCVNGNPWLAYWGQGTLV
TVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKRVEPKSCDKTHTCPPC
PAPEFEGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPASIEKTISKAKGQ
PREPQVCTLPPSREEMTKNQVSLSCAVKGFYPSDIAVEWESNGQPENNYKTTPPVLD
SDGSFFLVSKLTVDKSRWQQGNVFSCSVMHEALHNRFTQKSLSLSPGK
Gemtuzumab LC (SEQ ID NO: 1151):
DIQLTQSPSTLSASVGDRVTITCRASESLDNYGIRFLTWFQQKPGKAPKLLMYAASNQ
GSGVPSRFSGSGSGTEFTLTISSLQPDDFATYYCQQTKEVPWSFGQGTKVEVKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC

TABLE 7
Bispecific Antibody Expression Characteristics

| | | | SEC-HPLC | | |
|---|---|---|---|---|---|
| Bispecific | Anti-CD3 Antibody in | Titer | % | % | % |
| Antibody | Bispecific Antibody | (mg/L) | HMW | Main | LMW |
| CD3 × CD33- | ABL-CD3-13_scFv_VH-VL | 195.56 | 10.03 | 89 | 0.70 |
| BspAb1 | | | | | |
| CD3 × CD33- | ABL-CD3-16_(C to S HC)_scFv_VH-VL | 170.14 | 8.45 | 91 | 0.55 |
| BspAb2 | | | | | |
| CD3 × CD33- | ABL-CD3-16_(C to Y HC)_scFv_VH-VL | 111.05 | 5.84 | 93 | 0.69 |
| BspAb3 | | | | | |
| CD3 × CD33- | ABL-CD3-3_scFv_VH-VL | 80.51 | 6.51 | 93 | 0.92 |
| BspAb4 | | | | | |
| CD3 × CD33- | ABL-CD3-4_(E added to HC, G to C | 168.45 | 5.49 | 94 | 0.69 |
| BspAb5 | LC)_scFv_VH:VL | | | | |
| CD3 × CD33- | ABL-CD3-2_scFv_VH-VL | 31.82 | 11.76 | 87 | 0.86 |
| BspAb6 | | | | | |
| CD3 × CD33- | ABL-CD3-6_(NHS to QHS LC)_scFv- | 66.05 | 8.02 | 90 | 2.36 |
| BspAb7 | VH-VL | | | | |
| CD3 × CD33- | ABL-CD3-8_(NHS to QHS | 108.74 | 1.83 | 97 | 0.82 |
| BspAb8 | LC)_scFv_VH-VL | | | | |
| CD3 × CD33- | ABL-CD3-9_(NHC to QHS | 30.18 | 10.74 | 88 | 1.62 |
| BspAb9 | LC)_scFv_VH-VL | | | | |
| CD3 × CD33- | ABL-CD3-1_scFv_VH-VL | 143.75 | 3.29 | 96 | 1.04 |
| BspAb10 | | | | | |
| CD3 × CD33- | ABL-CD3-14_(C to S HC)_scFv_VH- | 24.65 | 21.48 | 78 | 0.94 |
| BspAb11 | VL | | | | |
| CD3 × CD33- | ABL-CD3-14_(C to F HC)_scFv_VH- | 113.41 | 3.17 | 96 | 0.91 |
| BspAb12 | VL | | | | |
| CD3 × CD33- | ABL-CD3-7_scFv_VH-VL | 277.35 | 8.48 | 90 | 1.81 |
| BspAb13 | | | | | |
| CD3 × CD33- | ABL-CD3-5_scFv-VH-VL | 184.59 | 7.42 | 92 | 0.83 |
| BspAb14 | | | | | |
| CD3 × CD33- | ABL-CD3-17_scFv_VH-VL | 59.54 | 7.19 | 91 | 1.52 |
| BspAb15 | | | | | |
| CD3 × CD33- | ABL-CD3-10 (C to S)_scFv_VH-VL | 141.18 | 1.44 | 97 | 1.33 |
| BspAb16 | | | | | |
| CD3 × CD33- | ABL-CD3-11_scFv_VH-VL | 165.12 | 4.07 | 95 | 0.82 |
| BspAb17 | | | | | |
| CD3 × CD33- | ABL-CD3-13_scFv_VH-VL | 123.76 | 23.85 | 75 | 0.66 |
| BspAb18 | | | | | |
| CD3 × CD33- | ABL-CD3-16 (C to S HC)_scFv_VL- | 161.94 | 18.32 | 81 | 1.04 |
| BspAb19 | VH | | | | |
| CD3 × CD33- | ABL-CD3-16 (C to Y HC)_scFv_VL- | 96.24 | 12.64 | 87 | 0.86 |
| BspAb20 | VH | | | | |
| CD3 × CD33- | ABL-CD-3-3_scFv_VL-VH | 112.38 | 8.67 | 90 | 1.16 |
| BspAb21 | | | | | |
| CD3 × CD33- | ABL-CD3-4_(E added to HC, G to C | 117.81 | 20.62 | 78 | 0.90 |
| BspAb22 | LC)_scFv_VL-VH | | | | |
| CD3 × CD33- | ABL-CD3-2_scFv_VL-VH | 123.14 | 6.81 | 93 | 0.69 |
| BspAb23 | | | | | |
| CD3 × CD33- | ABL-CD3-6 (NHS to QHS | 88.96 | 10.70 | 88 | 0.99 |
| BspAb24 | LC)_scFv_VL-VH | | | | |
| CD3 × CD33- | ABL-CD3-8 NHS to QHS | 109.82 | 5.83 | 94 | 0.55 |
| BspAb25 | LC)_scFv_VL-VH | | | | |
| CD3 × CD33- | ABL-CD3-9 (NHC to QHS | 21.37 | 24.55 | 58 | 17.32 |
| BspAb26 | LC)_scFv_VL-VH | | | | |
| CD3 × CD33- | ABL-CD3-1_scFv_VL-VH | 130.32 | 7.12 | 92 | 1.14 |
| BspAb27 | | | | | |
| CD3 × CD33- | ABL-CD3-14_(C to S HC)_scFv_VL- | 34.69 | 11.26 | 88 | 0.47 |
| BspAb28 | VH | | | | |
| CD3 × CD33- | ABL-CD3-14_(C to F HC)_scFv_VL- | 75.84 | 15.37 | 83 | 1.68 |
| BspAb29 | VH | | | | |
| CD3 × CD33- | ABL-CD3-7_scFv_VL-VH | 120.02 | 3.53 | 95 | 1.14 |
| BspAb30 | | | | | |
| CD3 × CD33- | ABL-CD3-5_scFv_VL-VH | 120.22 | 10.56 | 88 | 1.15 |
| BspAb31 | | | | | |
| CD3 × CD33- | ABL-CD3-17_scFv_VL-VH | 32.89 | 4.93 | 92 | 2.76 |
| BspAb32 | | | | | |
| CD3 × CD33- | ABL-CD3-10 (C to S)_scFv_VL-VH | 87.64 | 2.14 | 84 | 13.51 |
| BspAb33 | | | | | |
| CD3 × CD33- | ABL-CD3-11_scFv_VL-VH | 67.54 | 22 | 75 | 3.30 |
| BspAb34 | | | | | |
| CD3 × CD33- | ABL-CD3-13A_scFv_VH-VL | 39.53 | 2.36 | 92 | 5.60 |
| BspAb35 | | | | | |
| CD3 × CD33- | ABL-CD3-16A (C to S in | 63.75 | 3.91 | 91 | 4.76 |
| BspAb36 | HC)_scFv_VH-VL | | | | |
| CD3 × CD33- | ABL-CD3-16 (C to Y in | 48.79 | 2.4 | 93 | 4.81 |
| BspAb37 | HC)_scFv_VH-VL | | | | |
| CD3 × CD33- | ABL-CD3-3A_scFv_VH-VL | 59.41 | 1.23 | 95 | 3.95 |
| BspAb38 | | | | | |
| CD3 × CD33- | ABL-CD3-2A_scFv_VH-VL | 65.06 | 2.41 | 93 | 4.18 |
| BspAb39 | | | | | |
| CD3 × CD33- | ABL-CD3-6A (NHS to QHS in | 28.24 | 1.98 | 92 | 5.54 |
| BspAb40 | LC)_scFv_VH-VL | | | | |
| CD3 × CD33- | ABL-CD3-8A (NHS to QHS in | 25.40 | 0.05 | 96 | 4.38 |
| BspAb41 | LC)_scFv_VH-VL | | | | |
| CD3 × CD33- | ABL-CD3-1A_scFv_VH-VL | 54.03 | 2.37 | 93 | 4.16 |
| BspAb42 | | | | | |
| CD3 × CD33- | ABL-CD3-14A_(C to F | 39.53 | 0.92 | 95 | 4.22 |
| BspAb43 | HC)_scFv_VH-VL | | | | |
| CD3 × CD33- | ABL-CD3-7A_scFv_VH-VL | 49.79 | 1.46 | 94 | 4.90 |
| BspAb44 | | | | | |
| CD3 × CD33- | ABL-CD3-5A_scFv_VH-VL | 71.43 | 4.55 | 92 | 3.28 |
| BspAb45 | | | | | |
| CD3 × CD33- | ALB-CD3-17A_scFv_VH-VL | 23.72 | 1.33 | 96 | 3.13 |
| BspAb46 | | | | | |
| CD3 × CD33- | ABL-CD3-10A (C to S)_scFv_VH-VL | 31.90 | 3.01 | 89 | 8.41 |
| BspAb47 | | | | | |
| CD3 × CD33- | ABL-CD3-11A_scFv_VH-VL | 61.08 | 13.12 | 80 | 6.75 |
| BspAb48 | | | | | |
| CD3 × CD33- | ABL-CD3-13A_scFv_VL-VH | 31.90 | 6.15 | 89 | 4.76 |
| BspAb49 | | | | | |
| CD3 × CD33- | ABL-CD3-16A (C to S in | 61.44 | 9.21 | 85 | 5.52 |
| BspAb50 | HC)_scFv_VL-VH | | | | |
| CD3 × CD33- | ABL-CD3-16A (C to Y in | 46.17 | 2.29 | 92 | 5.66 |
| BspAb51 | HC)_scFv_VL-VH | | | | |
| CD3 × CD33- | ABL-CD-3A_scFv_VL-VH | 53.18 | 4.61 | 86 | 9.43 |
| BspAb52 | | | | | |
| CD3 × CD33- | ABL-CD3-4A_(E added to HC, G to C | 35.97 | 2.47 | 93 | 4.23 |
| BspAb53 | LC)_scFv_VL-VH | | | | |
| CD3 × CD33- | ABL-CD3-2A_scFv_VL-VH | 55.03 | 1.91 | 93 | 5.26 |
| BspAb54 | | | | | |
| CD3 × CD33- | ABL-CD3-6A (NHS to QHS in | 36.28 | 0.94 | 94 | 5.00 |
| BspAb55 | LC)_scFv_VL-VH | | | | |
| CD3 × CD33- | ABL-CD3-8A (NHS to QHS in | 30.91 | 2.62 | 94 | 3.64 |
| BspAb56 | LC)_scFv_VL-VH | | | | |
| CD3 × CD33- | ABL-CD3-9A (NHC to QHS in | 19.43 | 9.28 | 74 | 16.79 |
| BspAb57 | LC)_scFv_VL-VH | | | | |
| CD3 × CD33- | ABL-CD3-1A_scFv_VL-VH | 39.53 | 0.36 | 96 | 3.68 |
| BspAb58 | | | | | |
| CD3 × CD33- | ABL-CD3-14A (C to SHC)_scFv_VL- | 28.92 | 0.41 | 89 | 10.08 |
| BspAb59 | VH | | | | |
| CD3 × CD33- | ABL-CD3-14A (C to F HC)_scFv_VL- | 38.40 | 0.42 | 96 | 3.25 |
| BspAb60 | VH | | | | |
| CD3 × CD33- | ABL-CD3-7A_scFv_VL-VH | 48.70 | 0.58 | 94 | 5.28 |
| BspAb61 | | | | | |
| CD3 × CD33- | ABL-CD3-5A_scFv_VL-VH | 72.74 | 2.57 | 91 | 6.52 |
| BspAb62 | | | | | |
| CD3 × CD33- | ABL-CD3-17A_scFv_VL-VH | 22.01 | 2.61 | 92 | 5.82 |
| BspAb63 | | | | | |
| CD3 × CD33- | ABL-CD3-10A (C to S)_scFv_VL-VH | 51.78 | 0.74 | 16 | 83.62 |
| BspAb64 | | | | | |
| CD3 × CD33- | ABL-CD3-11A_scFv_VL-VH | 43.19 | 1.81 | 95 | 3.23 |
| BspAb65 | | | | | |
| CD3 × CD33- | SEQ ID NO: 1154, infra | 49.79 | 1 | 92 | 7.44 |
| BspAb66 | | | | | |
| CD3 × CD33- | ABL-CD3-45A_scFv_VH-VL | 29.25 | − | 100.00 | − |
| BspAb67 | | | | | |
| CD3 × CD33- | ABL-CD3-49A_scFv_VH-VL | 68.5 | 2.93 | 97.07 | − |
| BspAb68 | | | | | |
| CD3 × CD33- | ALB-CD3-52A_scFv_VH-VL | 29.92 | 4.85 | 95.16 | − |
| BspAb69 | | | | | |
| CD3 × CD33- | ABL-CD3-74A_scFv_VH-VL | 20.13 | − | 88.45 | − |
| BspAb70 | | | | | |
| CD3 × CD33- | ABL-CD3-21A_scFv_VH-VL | 25.13 | − | 100.00 | − |
| BspAb71 | | | | | |
| CD3 × CD33- | ABL-CD3-22A_scFv_VH-VL | 107.74 | 8.1 | 91.9 | − |
| BspAb72 | | | | | |
| CD3 × CD33- | ABL-CD3-41A_scFv_VH-VL | 64.84 | − | 100 | − |
| BspAb73 | | | | | |
| CD3 × CD33- | ABL-CD3-56A_scFv_VH-VL | 34.73 | − | 89.38 | 6.19 |
| BspAb74 | | | | | |
| CD3 × CD33- | ABL-CD3-85A_scFv_VH-VL | 31.48 | − | 95.03 | 4.97 |
| BspAb75 | | | | | |
| CD3 × CD33- | ABL-CD3-85B_scFv_VH-VL | 68.72 | 6.65 | 92.49 | − |
| BspAb76 | | | | | |
| CD3 × CD33- | ABL-CD3-72A_scFv_VH-VL | 72.75 | 5.17 | 94.82 | − |
| BspAb77 | | | | | |
| CD3 × CD33- | ABL-CD3-87A_scFv_VH-VL | 205.7 | 6.06 | 93.94 | − |
| BspAb78 | | | | | |
| CD3 × CD33- | ABL-CD3-66A_scFv_VH-VL | 172.92 | 3.06 | 96.94 | − |
| BspAb79 | | | | | |
| CD3 × CD33- | ABL-CD3-63A_scFv_VH-VL | 85.96 | − | 100 | − |
| BspAb80 | | | | | |
| CD3 × CD33- | ABL-CD3-81A_scFv_VH-VL | 81.53 | 6.11 | 93.88 | − |
| BspAb81 | | | | | |
| CD3 × CD33- | ABL-CD3-20A_scFv_VH-VL | 37.71 | − | 100 | − |
| BspAb82 | | | | | |
| CD3 × CD33- | ABL-CD3-56A_scFv_VH-VL | 32.82 | − | 100 | − |
| BspAb83 | | | | | |
| CD3 × CD33- | ABL-CD3-64A_scFv_VH-VL | 141.81 | 4.78 | 95.22 | − |
| BspAb84 | | | | | |
| CD3 × CD33- | ABL-CD3-62A_scFv_VH-VL | 168.96 | 7.09 | 92.91 | − |
| BspAb85 | | | | | |
| CD3 × CD33- | ABL-CD3-61A_scFv_VH-VL | 62.16 | − | 100 | − |
| BspAb86 | | | | | |
| CD3 × CD33- | ABL-CD3-79A_scFv_VH-VL | 41.05 | 14.05 | 85.67 | − |
| BspAb87 | | | | | |
| CD3 × CD33- | ABL-CD3-27A_scFv_VH-VL | 85.85 | 2.02 | 97.98 | − |
| BspAb88 | | | | | |
| CD3 × CD33- | ABL-CD3-89A_scFv_VH-VL | 79.73 | − | 100 | − |
| BspAb89 | | | | | |
| CD3 × CD33- | ABL-CD3-88A_scFv_VH-VL | 106.28 | 7.15 | 92.85 | − |
| BspAb90 | | | | | |
| CD3 × CD33- | ABL-CD3-36A_scFv_VL-VH | 60.06 | 2.73 | 97.27 | − |
| BspAb91 | | | | | |
| CD3 × CD33- | ABL-CD3-45A_scFv_VL-VH | 115.32 | 5.6 | 94.4 | − |
| BspAb92 | | | | | |
| CD3 × CD33- | ABL-CD3-47A_scFv_VL-VH | 27.4 | − | 100 | − |
| BspAb93 | | | | | |
| CD3 × CD33- | ABL-CD3-53A_scFv_VL-VH | 38.25 | − | 100 | − |
| BspAb94 | | | | | |
| CD3 × CD33- | ABL-CD3-49A_scFv_VL-VH | 115.44 | 1.36 | 98.64 | − |
| BspAb95 | | | | | |
| CD3 × CD33- | ABL-CD3-41A_scFv_VL-VH | 73.36 | − | 100 | − |
| BspAb96 | | | | | |
| CD3 × CD33- | ABL-CD3-76A_scFv_VL-VH | 124.86 | 12.75 | 87.25 | − |
| BspAb97 | | | | | |
| CD3 × CD33- | ABL-CD3-34A_scFv_VL-VH | 87.85 | − | 100 | − |
| BspAb98 | | | | | |
| CD3 × CD33- | ABL-CD3-37A_scFv_VL-VH | 43.74 | − | 97.6 | − |
| BspAb99 | | | | | |
| CD3 × CD33- | ABL-CD3-76A_scFv_VH-VL | 28.03 | − | 81.43 | − |
| BspAb100 | | | | | |
| CD3 × CD33- | ABL-CD3-37A_scFv_VH-VL | 26.87 | 6.36 | 93.64 | − |
| BspAb101 | | | | | |
| CD3 × CD33- | ABL-CD3-40A_scFv_VL-VH | 145.08 | 2.76 | 97.24 | − |
| BspAb102 | | | | | |
| CD3 × CD33- | ABL-CD3-40A_scFv_VH-VL | 75.24 | 11.55 | 88.44 | − |
| BspAb103 | | | | | |
| CD3 × CD33- | ABL-CD3-43A_scFv_VL-VH | 45.6 | 14.77 | 85.23 | − |
| BspAb104 | | | | | |
| CD3 × CD33- | ABL-CD3-52A_scFv_VL-VH | 56.38 | 4.43 | 95.57 | − |
| BspAb105 | | | | | |
| CD3 × CD33- | ABL-CD3-74A_scFv_VL-VH | 37.97 | − | 100 | − |
| BspAb106 | | | | | |
| CD3 × CD33- | ABL-CD3-21A_scFv_VL-VH | 34.65 | − | 100 | − |
| BspAb107 | | | | | |
| CD3 × CD33- | ABL-CD3-22A_scFv_VL-VH | 98.2 | 4.3 | 95.69 | − |
| BspAb108 | | | | | |
| CD3 × CD33- | ABL-CD3-68A_scFv_VL-VH | 22.91 | 13.37 | 86.63 | − |
| BspAb109 | | | | | |
| CD3 × CD33- | ABL-CD3-85A_scFv_VL-VH | 84.26 | 3.10 | 96.90 | − |
| BspAb110 | | | | | |
| CD3 × CD33- | ABL-CD3-85B_scFv_VL-VH | 86.29 | 3.95 | 96.05 | − |
| BspAb111 | | | | | |
| CD3 × CD33- | ABL-CD3-72A_scFv_VL-VH | 54.01 | 4.29 | 95.71 | − |
| BspAb112 | | | | | |
| CD3 × CD33- | ABL-CD3-87A_scFv_VL-VH | 293.89 | 18.96 | 81.04 | − |
| BspAb113 | | | | | |
| CD3 × CD33- | ABL-CD3-66A_scFv_VL-VH | 174.4 | 1.09 | 98.91 | − |
| BspAb114 | | | | | |
| CD3 × CD33- | ABL-CD3-63A_scFv_VL-VH | 86.22 | − | 100 | − |
| BspAb115 | | | | | |
| CD3 × CD33- | ABL-CD3-81A_scFv_VL-VH | 115.59 | 7.09 | 92.91 | − |
| BspAb116 | | | | | |
| CD3 × CD33- | ABL-CD3-20A_scFv_VL-VH | 74.25 | − | 100 | − |
| BspAb117 | | | | | |
| CD3 × CD33- | ABL-CD3-56A_scFv_VL-VH | 65.42 | 2.11 | 97.89 | − |
| BspAb118 | | | | | |
| CD3 × CD33- | ABL-CD3-64A_scFv_VL-VH | 148.46 | 10.41 | 89.58 | − |
| BspAb119 | | | | | |
| CD3 × CD33- | ABL-CD3-62A_scFv_VL-VH | 238.78 | 13.96 | 86.04 | − |
| BspAb120 | | | | | |
| CD3 × CD33- | ABL-CD3-61A_scFv_VL-VH | 36.55 | − | 100 | − |
| BspAb121 | | | | | |
| CD3 × CD33- | ABL-CD3-79A_scFv_VL-VH | 35.66 | 5.8 | 94.2 | − |
| BspAb122 | | | | | |
| CD3 × CD33- | ABL-CD3-27A_scFv_VL-VH | 50.01 | − | 100 | − |
| BspAb123 | | | | | |
| CD3 × CD33- | ABL-CD3-89A_scFv_VL-VH | 119.16 | − | 100 | − |
| BspAb124 | | | | | |
| CD3 × CD33- | ABL-CD3-88A_scFv_VL-VH | 84.92 | 12.13 | 87.88 | − |
| BspAb125 | | | | | |
| CD3 × CD33- | ABL-CD3-71A_scFv_VH-VL | 75.3 | 4.45 | 95.55 | — |
| BspAb126 | | | | | |

As controls, scFvs were generated for high and low affinity versions of a humanized anti-CD3 antibody, as well as a CD3δε-specific control antibody. These sequences were fused to knob Fc and expressed as described for other anti-CD3 antibodies above.

Control Humanized anti-CD3 (high affinity)
scFv-Fc knob/TM (SEQ ID NO: 1152):
EVQLVESGGGLVQPGGSLRLSCAASGFTFSTYAMNWVRQAPGKGLEWVGRIRSKY
NNYATYYADSVKGRFTISRDDSKNTLYLQMNSLRAEDTAVYYCVRHGNFGDSYVS
WFAYWGQGTLVTVSSGKPGSGKPGSGKPGSGKPGSQAVVTQEPSLTVSPGGTVTLT
CGSSTGAVTTSNYANWVQQKPGKSPRGLIGGTNKRAPGVPARFSGSLLGGKAALTIS
GAQPEDEADYYCALWYSNHWVFGGGTKLTVLGGGGSDKTHTCPPCPAPEFEGGPS
VFLFPPKPKDTLMISRTPEVTCVVVDVSHEDPEVKFNWYVDGVEVHNAKTKPREEQ
YNSTYRVVSVLTVLHQDWLNGKEYKCKVSNKALPASIEKTISKAKGQPREPQVYTL
PPCREEMTKNQVSLWCLVKGFYPSDIAVEWESNGQPENNYKTTPPVLDSDGSFFLYS
KLTVDKSRWQQGNVFSCSVMHEALHNHYTQKSLSLSPGK
Control Humanized anti-CD3 (low affinity)
scFv-Fc knob/TM (SEQ ID NO: 1153):
EVQLVESGGGLVQPGGSLRLSCAASGFTFSTYAMNWVRQAPGKGLEWVGRIRSKY
NNYATYYADSVKGRFTISRDDSKNTLYLQMNSLRAEDTAVYYCVRHGNFGDSYVS
WFDYWGQGTLVTVSSGKPGSGKPGSGKPGSGKPGSQAVVTQEPSLTVSPGGTVTLT
CGSSTGAVTTSNYANWVQQKPGKSPRGLIGGTNKRAPGVPARFSGSLLGGKAALTIS
GAQPEDEADYYCALWYSNHWVFGGGTKLTVLGGGGSDKTHTCPPCPAPEFEGGPS
VFLFPPKPKDTLMISRTPEVTCVVVDVSHEDPEVKFNWYVDGVEVHNAKTKPREEQ
YNSTYRVVSVLTVLHQDWLNGKEYKCKVSNKALPASIEKTISKAKGQPREPQVYTL
PPCREEMTKNQVSLWCLVKGFYPSDIAVEWESNGQPENNYKTTPPVLDSDGSFFLYS
KLTVDKSRWQQGNVFSCSVMHEALHNHYTQKSLSLSPGK
CD3δε-specific control antibody scFv-Fc knob/TM
(CD3xCD33-BspAb66) (SEQ ID NO: 1154):
EVQLVESGGGLVQPGRSLRLSCAASGFTFDDYAMHWVRQAPGKGLEWVSGISWNS
GSIGYADSVKGRFTISRDNAKNSLYLQMNSLRAEDTALYYCAKDSRGYGDYRLGGA
YWGQGTLVTVSSGGGGSGGGGSGGGGSGGGGSEIVMTQSPATLSVSPGERATLSCR
ASQSVSSNLAWYQQKPGQAPRLLIYGASTRATGIPARFSGSGSGTEFTLTISSLQSEDF
AVYYCQQYNNWPWTFGQGTKVEIKTSGPGGPDKTHTCPPCPAPEFEGGPSVFLFPPK
PKDTLMISRTPEVTCVVVDVSHEDPEVKFNWYVDGVEVHNAKTKPREEQYNSTYRV
VSVLTVLHQDWLNGKEYKCKVSNKALPASIEKTISKAKGQPREPQVYTLPPCREEMT
KNQVSLWCLVKGFYPSDIAVEWESNGQPENNYKTTPPVLDSDGSFFLYSKLTVDKSR
WQQGNVFSCSVMHEALHNHYTQKSLSLSPGK

### Expression and Purification of Bispecific Constructs

Antibodies were expressed in Expi293 (HEK) cells following Thermofisher guidelines for the Expi293 Expression System (Publication number MAN0007814). Antibodies were expressed in 30 mL cultures in 125 mL Thomson Flasks (PN: 931110).

Antibodies were harvested after 4 days of expression by adding diatomaceous earth into the expression flask and filtered using 0.2 um Steriflips (Millipore PN: SCGP00525). The collected supernatants were purified on an Akta Pure using a 1 mL HiTrap MabSelect SuRe column with a loading flow rate of 4 mL/min. Once loaded, the column was washed with 5 CV of PBS and antibodies were eluted with 50 mM sodium acetate pH 3.5 and neutralized with 1 M tris pH 8.0. After neutralization antibodies were buffer exchanged into 1×PBS pH 7.4 using 50 kD MWCO spin concentrators (Sartorious PN:VS0632) by concentrating the neutralized eluted antibodies then filling the spin concentrator to the 6 mL mark and concentrating again. This was repeated 3 times to ensure complete buffer exchange.

After the antibodies were eluted the concentration and yield was determined by NanoDrop. Antibody quality was analyzed by SEC-HPLC on a TSKgel SuperSW3000 (4.6 mm ID×30 cm, 4 um, Tosoh PN: 18675) column equipped with a guard column (Tosoh) using 0.1 M NaPO4 and 0.1 M NaSO4 pH 6.8 at a flow rate of 0.35 mL/min. Peak analysis was done using CDS Open Lab 2.6.

### Binding Assays Performed Using Bispecific Antibodies

A panel of bispecific antibodies was assessed for binding specificity by flow cytometry using Jurkat WT versus Jurkat TCRb knockout cells, Pan-T cells, and CD33 expressing cells using Goat anti-human IgG Fc (Jackson ImmunoReasearch Labs, Inc., West Grove, PA, cat #109-605-098) (Table 8, infra). In addition, a subset of bispecific antibodies was tested for binding to recombinant CD3 proteins by Octet (Table 9, infra).

TABLE 8
Binding of Bispecific Antibodies by Flow Cytometry.

| | Jurkat | Jurkat | KILR- | Jurkat | Jurkat | KILR- |
|---|---|---|---|---|---|---|
| | WT at | KO at | HL60 at | WT at | KO at | HL60 at |
| Bispecific | 10 | 10 | 10 | 1 | 1 | 1 |
| Antibody | ug/ml | ug/ml | ug/ml | ug/ml | ug/ml | ug/ml |
| CD3 × CD33- | 26107 | 3547 | 746131 | 12459 | 2875 | 569075 |
| BspAb36 | | | | | | |
| CD3 × CD33- | 34072 | 3720 | 476341 | 23699 | 3339 | 635122 |
| BspAb38 | | | | | | |
| CD3 × CD33- | 2736 | 2393 | 572212 | 2155 | 2604 | 540698 |
| BspAb39 | | | | | | |
| CD3 × CD33- | 26425 | 3292 | 485364 | 12154 | 2966 | 453491 |
| BspAb42 | | | | | | |
| CD3 × CD33- | 84844 | 6435 | 475073 | 113405 | 7549 | 639856 |
| BspAb44 | | | | | | |
| CD3 × CD33- | 38314 | 3638 | 741077 | 19214 | 3664 | 640144 |
| BspAb45 | | | | | | |
| CD3 × CD33- | 24289 | 3844 | 690666 | 11987 | 3008 | 509216 |
| BspAb51 | | | | | | |
| CD3 × CD33- | 32767 | 3558 | 757807 | 10922 | 2933 | 486159 |
| BspAb37 | | | | | | |
| CD3 × CD33- | 10810 | 2604 | 482530 | 4140 | 2414 | 566282 |
| BspAb48 | | | | | | |
| CD3 × CD33- | 33901 | 3403 | 464863 | 9701 | 2863 | 475294 |
| BspAb50 | | | | | | |
| CD3 × CD33- | 31959 | 3808 | 651570 | 15287 | 3491 | 756118 |
| BspAb52 | | | | | | |
| CD3 × CD33- | 84839 | 4825 | 537674 | 95572 | 6023 | 621381 |
| BspAb61 | | | | | | |
| CD3 × CD33- | 59000 | 6093 | 594872 | 62472 | 5298 | 294996 |
| BspAb62 | | | | | | |
| CD3 × CD33- | 3216 | 2250 | 605191 | 2363 | 2297 | 533387 |
| BspAb65 | | | | | | |
| CD3 × CD33- | 23096 | 3039 | 499348 | 6659 | 2681 | 569137 |
| BspAb66 | | | | | | |
| High affinity | 38390 | 5314 | 647770 | 37881 | 5481 | 543891 |
| Control | | | | | | |
| Low affinity | 3227 | 2467 | 622703 | 2616 | 2822 | 534945 |
| Control | | | | | | |
| Gemtuzumab | 2569 | 2818 | 351424 | 2574 | 3003 | 320644 |
| hIgG1 | 2391 | 2620 | 30195 | 2700 | 2830 | 34335 |
| No mAb | 2358 | 2529 | 7580 | 2659 | 2882 | 7691 |
| OKT3 | 35846 | 3677 | 11587 | 82070 | 5185 | 14784 |

TABLE 9
Binding of Bispecific Antibodies By Octet.

| Bispecific | | KD | | | Full |
|---|---|---|---|---|---|
| Antibody | Response | (M) | kon(1/Ms) | kdis(1/s) | R\hat{ }2 |
| CD3 × CD33- | 0.42 | 2.05E−07 | 1.63E+04 | 3.34E−03 | 1.00 |
| BspAb36 | | | | | |
| CD3 × CD33- | 0.28 | 8.10E−08 | 2.50E+05 | 2.03E−02 | 0.98 |
| BspAb38 | | | | | |
| CD3 × CD33- | 0.10 | 2.99E−07 | 3.91E+05 | 1.17E−01 | 0.96 |
| BspAb39 | | | | | |
| CD3 × CD33- | 0.51 | 1.33E−07 | 1.39E+05 | 1.86E−02 | 1.00 |
| BspAb42 | | | | | |
| CD3 × CD33- | 0.56 | 4.85E−09 | 3.27E+05 | 1.59E−03 | 0.99 |
| BspAb44 | | | | | |
| CD3 × CD33- | 0.58 | 1.77E−07 | 2.99E+05 | 5.29E−02 | 1.00 |
| BspAb45 | | | | | |
| CD3 × CD33- | 0.26 | 7.40E−08 | 3.14E+04 | 2.32E−03 | 0.98 |
| BspAb51 | | | | | |
| CD3 × CD33- | 0.34 | 2.13E−07 | 1.58E+04 | 3.36E−03 | 1.00 |
| BspAb37 | | | | | |
| CD3 × CD33- | 0.23 | 6.14E−08 | 3.51E+04 | 2.16E−03 | 0.98 |
| BspAb48 | | | | | |
| CD3 × CD33- | 0.33 | 1.25E−07 | 2.10E+04 | 2.62E−03 | 0.99 |
| BspAb50 | | | | | |
| CD3 × CD33- | 0.07 | 1.95E−08 | 3.86E+05 | 7.51E−03 | 0.97 |
| BspAb52 | | | | | |
| CD3 × CD33- | 0.74 | 7.62E−09 | 2.45E+05 | 1.87E−03 | 0.99 |
| BspAb61 | | | | | |
| CD3 × CD33- | 0.25 | 8.59E−08 | 5.52E+05 | 4.74E−02 | 0.95 |
| BspAb62 | | | | | |
| CD3 × CD33- | 0.09 | 1.17E−08 | 1.14E+05 | 1.34E−03 | 0.95 |
| BspAb65 | | | | | |
| CD3 × CD33- | 0.03 | 8.54E−10 | 1.25E+04 | 1.07E−05 | 0.00 |
| BspAb66 | | | | | |
| High affinity | 0.73 | 2.01E−08 | 3.25E+05 | 6.52E−03 | 0.99 |
| Control | | | | | |
| Low affinity | 0.30 | 5.03E−08 | 2.41E+05 | 1.21E−02 | 0.93 |
| Control | | | | | |

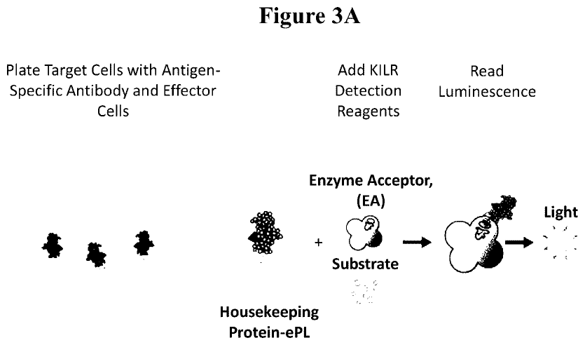

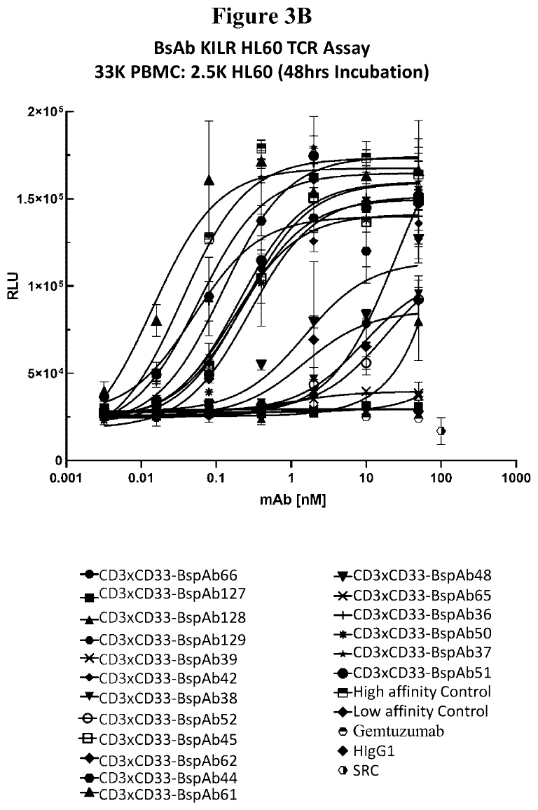

A panel of bispecific antibodies was selected for further characterization in additional assays to assess their functionality and toxicity. Killing Immune-Lysis Reaction (KILR) T Cell Redirection (TCR) assay (DiscoverX KILR HL-60 TCR Cytotoxic Assay) evaluated the ability of the bispecific anti-CD3 antibodies to mediate an interaction between effector (human Pan-T or PBMC) and target (CD33) expressing cells resulting in the release of reporter element as well as in cellular toxicity (FIG. 3A and FIG. 3B, Table 10, infra).

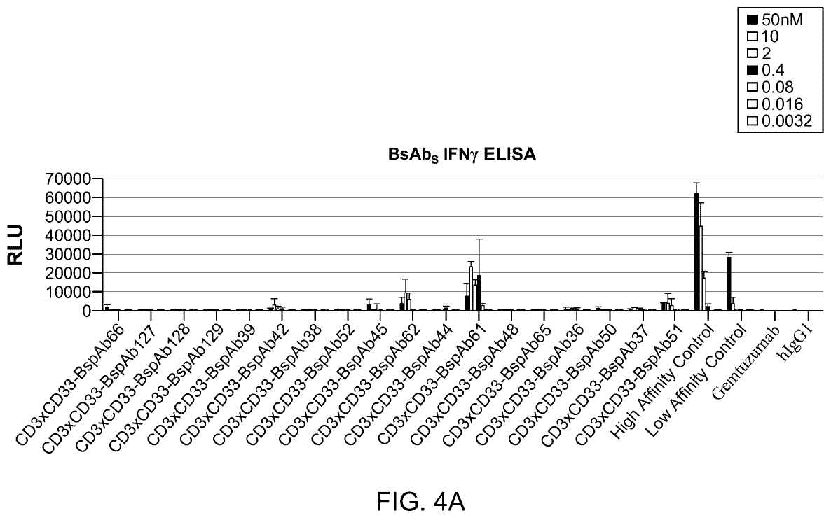

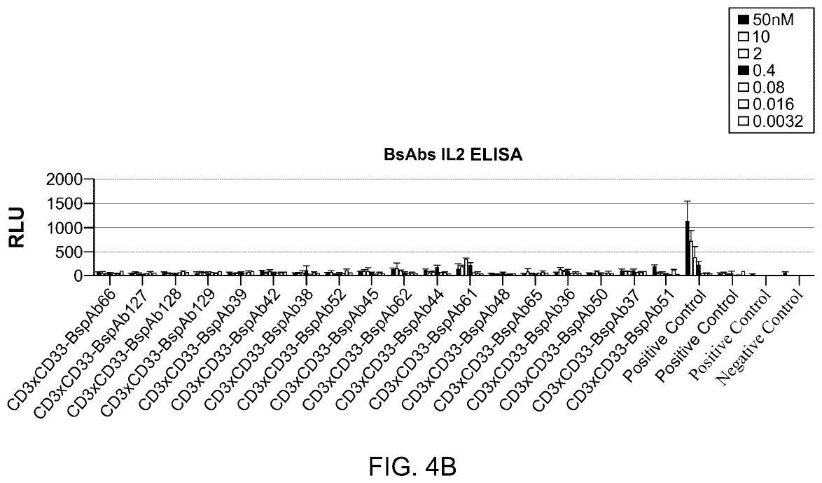

To generate a panel of bispecific anti-CD3 antibodies with good cytotoxic capacity yet minimal cytokine induction, we tested the supernatants of the effector human Pan-T or PBMC cells in quantitative ELISA against IL2 and IFNγ (FIG. 4A and FIG. 4B, Table 10, infra).

TABLE 10
RTCC Assay and Cytokine Release

RTCC

| | EC₅₀ | | IFNg | IL2 |
|---|---|---|---|---|
| Bispecific Antibody | (nM) | R\hat{ }2 | 1 to 4 | 1 to 45 |
| CD3 × CD33- | 0.06 | 0.9 | 1374 | 40 |
| BspAb36 | | | | |
| CD3 × CD33- | 9.29 | 1.0 | 44 | 56 |
| BspAb38 | | | | |
| CD3 × CD33- | ND | 0.9 | 17 | 53 |
| BspAb39 | | | | |
| CD3 × CD33- | 0.17 | 1.0 | 284 | 110 |
| BspAb42 | | | | |
| CD3 × CD33- | 0.05 | 1.0 | 337 | 106 |
| BspAb44 | | | | |
| CD3 × CD33- | 0.22 | 1.0 | 821 | 76 |
| BspAb45 | | | | |
| CD3 × CD33- | 0.20 | 0.9 | 3341 | 170 |
| BspAb51 | | | | |
| CD3 × CD33- | 0.12 | 0.9 | 604 | 76 |
| BspAb37 | | | | |
| CD3 × CD33- | 1.50 | 0.9 | 97 | 33 |
| BspAb48 | | | | |
| CD3 × CD33- | 0.28 | 1.0 | 1991 | 63 |
| BspAb50 | | | | |
| CD3 × CD33- | 17.81 | 1.0 | 94 | 53 |
| BspAb52 | | | | |
| CD3 × CD33- | 0.01 | 1.0 | 12301 | 216 |
| BspAb61 | | | | |
| CD3 × CD33- | 0.24 | 1.0 | 1161 | 120 |
| BspAb62 | | | | |
| CD3 × CD33- | ND | 0.9 | 21 | 46 |
| BspAb65 | | | | |
| CD3 × CD33- | 25.45 | 1.0 | 2261 | 86 |
| BspAb66 | | | | |
| High affinity | 0.03 | 0.97 | 59376 | 843 |
| Control | | | | |
| Low affinity | 1.48 | 0.92 | 26748 | 33 |
| Control | | | | |

### Binding Assays Performed Using Bispecific Antibodies

A panel of bispecific antibodies was assessed for binding specificity by flow cytometry using Jurkat WT versus Jurkat TCRb knockout cells, Pan-T cells, and CD33 expressing cells using Goat anti-human IgG Fc (Jackson ImmunoReasearch Labs, Inc., West Grove, PA, cat #109-605-098) (Table 11, infra). In addition, a subset of bispecific antibodies was tested for binding to recombinant CD3 proteins by Octet (Table 12, infra).

TABLE 11
Binding of Bispecific Antibodies by Flow Cytometry.

| | | | | | Jurkat | Jurkat | Jurkat | Jurkat | Jurkat | Jurkat |
|---|---|---|---|---|---|---|---|---|---|---|
| | Pan-T | Pan-T | Pan-T | Pan-T | WT | WT | KO | KO | WT/KO | WT/KO |
| | (gMFI), | (% Positive, | (gMFI), | (% Positive, | (gMFI), | (gMFI), | (gMFI), | (gMFI), | (Fold), | (Fold), |
| BiSAb ID: | 10 ug/mL | 10 ug/mL | 1 ug/mL | 1 ug/mL | 10 ug/mL | 1 ug/mL | 10 ug/mL | 1 ug/mL | 10 ug/mL | 1 ug/mL |
| CD3 × CD33-BspAb67 | 673309 | 99.9 | 450196 | 99.8 | 66711 | 57105 | 4498 | 4034 | 14.8 | 14.2 |
| CD3 × CD33-BspAb68 | 600836 | 99.9 | 539367 | 100.0 | 63582 | 58008 | 4675 | 4500 | 13.6 | 12.9 |
| CD3 × CD33-BspAb69 | 281479 | 99.8 | 95339 | 99.7 | 25791 | 16481 | 2951 | 2602 | 8.7 | 6.3 |
| CD3 × CD33-BspAb70 | 141859 | 99.6 | 33142 | 99.2 | 17127 | 7272 | 2791 | 2274 | 6.1 | 3.2 |
| CD3 × CD33-BspAb71 | 7092 | 50.1 | 2329 | 1.7 | 2899 | 1671 | 1711 | 1814 | 1.7 | 0.9 |
| CD3 × CD33-BspAb72 | 8345 | 66.1 | 2427 | 2.0 | 3760 | 1951 | 1903 | 1926 | 2.0 | 1.0 |
| CD3 × CD33-BspAb73 | 22007 | 95.9 | 4182 | 17.3 | 6106 | 2509 | 2049 | 2106 | 3.0 | 1.2 |
| CD3 × CD33-BspAb74 | 25973 | 99.0 | 4348 | 16.4 | 7991 | 2766 | 2208 | 2191 | 3.6 | 1.3 |
| CD3 × CD33-BspAb75 | 148910 | 99.7 | 69762 | 99.2 | 15894 | 11261 | 2215 | 2114 | 7.2 | 5.3 |
| CD3 × CD33-BspAb76 | 43375 | 99.5 | 6793 | 50.2 | 9785 | 3178 | 2214 | 2079 | 4.4 | 1.5 |
| CD3 × CD33-BspAb77 | 188288 | 99.8 | 37766 | 92.5 | 22070 | 8960 | 2579 | 2464 | 8.6 | 3.6 |
| CD3 × CD33-BspAb78 | 224766 | 99.8 | 66458 | 98.0 | 28845 | 20346 | 2924 | 3085 | 9.9 | 6.6 |
| CD3 × CD33-BspAb79 | 166057 | 99.4 | 34696 | 99.3 | 20380 | 8833 | 2299 | 2225 | 8.9 | 4.0 |
| CD3 × CD33-BspAb80 | 485569 | 99.8 | 330002 | 99.7 | 59433 | 50541 | 3965 | 3861 | 15.0 | 13.1 |
| CD3 × CD33-BspAb81 | 368580 | 99.8 | 79909 | 99.7 | 55069 | 24138 | 4067 | 3402 | 13.5 | 7.1 |
| CD3 × CD33-BspAb82 | 462151 | 99.9 | 318963 | 99.5 | 58591 | 54545 | 4060 | 4228 | 14.4 | 12.9 |
| CD3 × CD33-BspAb83 | 597540 | 99.8 | 566077 | 99.8 | 69086 | 67667 | 4832 | 4820 | 14.3 | 14.0 |
| CD3 × CD33-BspAb84 | 362037 | 99.9 | 173191 | 99.9 | 50957 | 35248 | 3678 | 3250 | 13.9 | 10.8 |
| CD3 × CD33-BspAb85 | 249316 | 99.7 | 95995 | 99.4 | 31972 | 18920 | 3237 | 3061 | 9.9 | 6.2 |
| CD3 × CD33-BspAb86 | 25415 | 92.4 | 5409 | 35.2 | 9551 | 3175 | 2450 | 2289 | 3.9 | 1.4 |
| CD3 × CD33-BspAb87 | 3118 | 4.8 | 1979 | 1.6 | 2177 | 1607 | 1718 | 1909 | 1.3 | 0.8 |
| CD3 × CD33-BspAb88 | 2056 | 1.5 | 2098 | 1.8 | 1801 | 1698 | 1860 | 2102 | 1.0 | 0.8 |
| CD3 × CD33-BspAb89 | 1960 | 1.7 | 1832 | 0.6 | 1751 | 1843 | 1896 | 2237 | 0.9 | 0.8 |
| CD3 × CD33-BspAb90 | 78440 | 98.1 | 11621 | 83.8 | 19388 | 4480 | 2574 | 2368 | 7.5 | 1.9 |
| CD3 × CD33-BspAb91 | 127228 | 99.4 | 18485 | 94.6 | 31551 | 8109 | 3025 | 2206 | 10.4 | 3.7 |
| CD3 × CD33-BspAb92 | 94733 | 99.8 | 12057 | 86.5 | 24788 | 4957 | 3037 | 2291 | 8.2 | 2.2 |
| CD3 × CD33-BspAb93 | 135405 | 99.8 | 29610 | 98.2 | 28421 | 16200 | 2981 | 2901 | 9.5 | 5.6 |
| CD3 × CD33-BspAb94 | 2599 | 4.4 | 2054 | 2.6 | 2189 | 1833 | 2021 | 2198 | 1.1 | 0.8 |
| CD3 × CD33-BspAb95 | 232496 | 99.8 | 111514 | 99.7 | 39483 | 33943 | 3148 | 3243 | 12.5 | 10.5 |
| CD3 × CD33-BspAb96 | 17933 | 95.8 | 3695 | 8.2 | 7474 | 2369 | 2154 | 2078 | 3.5 | 1.1 |
| CD3 × CD33-BspAb97 | 53016 | 98.5 | 9272 | 66.3 | 8453 | 3893 | 2259 | 2331 | 3.7 | 1.7 |
| CD3 × CD33-BspAb98 | 277636 | 99.6 | 196583 | 99.8 | 47686 | 43454 | 3759 | 3869 | 12.7 | 11.2 |
| CD3 × CD33-BspAb99 | 1830 | 1.5 | 1807 | 1.2 | 1670 | 1658 | 1719 | 1771 | 1.0 | 0.9 |
| CD3 × CD33-BspAb100 | 347595 | 99.5 | 239825 | 99.5 | 39559 | 29953 | 3329 | 3211 | 11.9 | 9.3 |
| CD3 × CD33-BspAb101 | 2146 | 1.6 | 1909 | 1.8 | 1942 | 1776 | 2072 | 2286 | 0.9 | 0.8 |
| CD3 × CD33-BspAb102 | 2189 | 1.1 | 1990 | 1.1 | 2083 | 1854 | 1968 | 2281 | 1.1 | 0.8 |
| CD3 × CD33-BspAb103 | 2022 | 1.4 | 1886 | 1.8 | 1671 | 1612 | 1980 | 1896 | 0.8 | 0.9 |
| CD3 × CD33-BspAb104 | 2089 | 1.6 | 2023 | 1.2 | 1789 | 1705 | 1854 | 2138 | 1.0 | 0.8 |
| CD3 × CD33-BspAb105 | 10100 | 69.8 | 2770 | 4.4 | 3883 | 2185 | 2164 | 2340 | 1.8 | 0.9 |
| CD3 × CD33-BspAb106 | 1960 | 2.0 | 2001 | 1.2 | 1904 | 1841 | 2263 | 2285 | 0.8 | 0.8 |
| CD3 × CD33-BspAb107 | 1834 | 1.5 | 1848 | 1.6 | 1604 | 1688 | 1809 | 1841 | 0.9 | 0.9 |
| CD3 × CD33-BspAb108 | 1961 | 1.1 | 2011 | 1.0 | 1660 | 1701 | 1906 | 2037 | 0.9 | 0.8 |
| CD3 × CD33-BspAb109 | 1936 | 1.5 | 1868 | 1.7 | 1933 | 1776 | 2261 | 2236 | 0.9 | 0.8 |
| CD3 × CD33-BspAb110 | 8491 | 55.1 | 2855 | 8.3 | 4151 | 2361 | 2256 | 2272 | 1.8 | 1.0 |
| CD3 × CD33-BspAb111 | 68890 | 99.2 | 23079 | 92.3 | 10630 | 7100 | 2219 | 2176 | 4.8 | 3.3 |
| CD3 × CD33-BspAb112 | 23571 | 91.4 | 5265 | 32.6 | 6152 | 3022 | 2190 | 2161 | 2.8 | 1.4 |
| CD3 × CD33-BspAb113 | 110675 | 99.5 | 24919 | 96.2 | 16049 | 10093 | 2704 | 2570 | 5.9 | 3.9 |
| CD3 × CD33-BspAb114 | 5987 | 39.9 | 2576 | 2.6 | 3212 | 2108 | 2169 | 2136 | 1.5 | 1.0 |
| CD3 × CD33-BspAb115 | 34205 | 95.5 | 8663 | 63.4 | 12577 | 7379 | 2215 | 2064 | 5.7 | 3.6 |
| CD3 × CD33-BspAb116 | 29960 | 98.2 | 5262 | 31.6 | 9604 | 3317 | 2319 | 2085 | 4.1 | 1.6 |
| CD3 × CD33-BspAb117 | 194524 | 99.7 | 70309 | 99.6 | 38621 | 29655 | 3657 | 3410 | 10.6 | 8.7 |
| CD3 × CD33-BspAb118 | 370298 | 99.8 | 330690 | 99.3 | 64960 | 56289 | 4698 | 4562 | 13.8 | 12.3 |
| CD3 × CD33-BspAb119 | 166195 | 99.7 | 88220 | 99.8 | 25658 | 22416 | 2836 | 2860 | 9.0 | 7.8 |
| CD3 × CD33-BspAb120 | 87708 | 99.3 | 27621 | 95.2 | 14596 | 8733 | 2380 | 2348 | 6.1 | 3.7 |
| CD3 × CD33-BspAb121 | 1918 | 1.5 | 1921 | 1.5 | 2043 | 1924 | 2020 | 2301 | 1.0 | 0.8 |
| CD3 × CD33-BspAb122 | 2024 | 1.0 | 2077 | 1.9 | 1980 | 1910 | 2001 | 2269 | 1.0 | 0.8 |
| CD3 × CD33-BspAb123 | 1915 | 1.4 | 1863 | 1.8 | 1706 | 1675 | 2115 | 1920 | 0.8 | 0.9 |
| CD3 × CD33-BspAb124 | 1967 | 1.0 | 1946 | 1.5 | 1792 | 1799 | 1962 | 1984 | 0.9 | 0.9 |
| CD3 × CD33-BspAb125 | 28659 | 95.4 | 6023 | 42.2 | 13694 | 3683 | 2607 | 2382 | 5.3 | 1.5 |
| CD3 × CD33-BspAb126 | 161104 | 99.7 | 55991 | 98.3 | 26033 | 15687 | 2973 | 2954 | 8.8 | 5.3 |
| CD3εδ -specific | 3237 | 9.9 | 1979 | 0.8 | 2183 | 1703 | 1830 | 1914 | 1.2 | 0.9 |
| monovalent control | | | | | | | | | | |
| antibody | | | | | | | | | | |
| CD3εδ -specific | 147432 | 98.4 | 86064 | 97.2 | 13977 | 10936 | 2322 | 2411 | 6.0 | 4.5 |
| bivalent control | | | | | | | | | | |
| antibody | | | | | | | | | | |
| No Ab | 1852 | 0.7 | 1667 | 1.3 | 1892 | 1924 | 2011 | 2206 | 0.9 | 0.9 |
| No Ab | 2006 | 1.0 | 1846 | 0.7 | 1820 | 1965 | 1970 | 2224 | 0.9 | 0.9 |
| CD3 × CD33-BspAb44 | 507822 | 99.5 | 474542 | 99.5 | 54168 | 57504 | 4183 | 4399 | 12.9 | 13.1 |
| CD3 × CD33-BspAb48 | 12235 | 82.2 | 2867 | 3.4 | 5169 | 2260 | 2045 | 2025 | 2.5 | 1.1 |
| High affinity Control | 177899 | 99.4 | 66401 | 99.4 | 21587 | 15533 | 4183 | 3930 | 5.2 | 4.0 |
| Low affinity Control | 3406 | 7.9 | 2069 | 1.5 | 2094 | 1919 | 2002 | 2165 | 1.0 | 0.9 |
| Gemtuzumab | 1677 | 0.7 | 1771 | 1.2 | 1688 | 1654 | 1834 | 1856 | 0.9 | 0.9 |
| hIgG1 | 1783 | 0.8 | 1863 | 0.8 | 1742 | 1774 | 1862 | 2024 | 0.9 | 0.9 |
| OKT3 | 394453 | 99.9 | 602598 | 99.6 | 45189 | 54040 | 4071 | 4900 | 11.1 | 11.0 |
| FACS Buffer | 2061 | 1.1 | 1977 | 1.3 | 1858 | 1866 | 1950 | 2228 | 1.0 | 0.8 |

TABLE 12
Binding of Bispecific Antibodies By Octet.

| | Response | KD | | | Full |
|---|---|---|---|---|---|
| BiSAb ID | (800 nM) | (M) | kon(1/Ms) | kdis(1/s) | R\hat{ }2 |
| CD3 × CD33-BspAb67 | 0.42 | 5.57E−09 | 4.21E+05 | 2.34E−03 | 0.9882 |
| CD3 × CD33-BspAb68 | 0.70 | 7.09E−09 | 4.00E+05 | 2.83E−03 | 0.9727 |
| CD3 × CD33-BspAb69 | 0.25 | 4.01E−07 | 3.47E+05 | 1.39E−01 | 0.9948 |
| CD3 × CD33-BspAb70 | 0.10 | 9.11E−09 | 6.09E+05 | 5.54E−03 | 0.8253 |
| CD3 × CD33-BspAb71 | 0.09 | 2.46E−06 | 8.13E+04 | 2.00E−01 | 0.9728 |
| CD3 × CD33-BspAb72 | 0.04 | 8.27E−09 | 2.15E+05 | 1.78E−03 | 0.9244 |
| CD3 × CD33-BspAb73 | 0.14 | 4.88E−07 | 5.01E+05 | 2.44E−01 | 0.9848 |
| CD3 × CD33-BspAb74 | 0.00 | <1.0E−12 | 1.25E+04 | <1.0E−07 | 0 |
| CD3 × CD33-BspAb75 | 0.17 | 2.59E−07 | 2.77E+05 | 7.19E−02 | 0.9371 |
| CD3 × CD33-BspAb76 | 0.30 | 2.25E−07 | 3.28E+05 | 7.37E−02 | 0.9928 |
| CD3 × CD33-BspAb77 | 0.25 | 6.09E−07 | 3.63E+05 | 2.21E−01 | 0.9973 |
| CD3 × CD33-BspAb78 | 0.39 | 1.17E−07 | 3.01E+05 | 3.52E−02 | 0.9946 |
| CD3 × CD33-BspAb79 | 0.35 | 4.56E−07 | 1.82E+05 | 8.29E−02 | 0.997 |
| CD3 × CD33-BspAb80 | 0.44 | 6.06E−09 | 3.08E+05 | 1.86E−03 | 0.9905 |
| CD3 × CD33-BspAb81 | 0.35 | 6.94E−08 | 1.14E+05 | 7.91E−03 | 0.9881 |
| CD3 × CD33-BspAb82 | 0.44 | 1.87E−08 | 2.87E+05 | 5.36E−03 | 0.987 |
| CD3 × CD33-BspAb83 | 0.55 | 3.28E−09 | 4.08E+05 | 1.34E−03 | 0.9778 |
| CD3 × CD33-BspAb84 | 0.52 | 2.46E−08 | 2.73E+05 | 6.71E−03 | 0.9897 |
| CD3 × CD33-BspAb85 | 0.57 | 6.08E−08 | 2.46E+05 | 1.49E−02 | 0.9829 |
| CD3 × CD33-BspAb86 | 0.03 | 5.42E−08 | 2.76E+05 | 1.49E−02 | 0.9599 |
| CD3 × CD33-BspAb87 | 0.01 | 5.17E−09 | 5.94E+05 | 3.07E−03 | 0.5677 |
| CD3 × CD33-BspAb88 | 0.29 | 1.26E−08 | 6.18E+04 | 7.78E−04 | 0.9688 |
| CD3 × CD33-BspAb89 | 0.01 | 2.44E−08 | 2.37E+06 | 5.78E−02 | 0.8423 |
| CD3 × CD33-BspAb90 | 0.22 | 3.34E−07 | 1.38E+04 | 4.59E−03 | 0.9968 |
| CD3 × CD33-BspAb91 | 0.37 | 6.38E−08 | 3.19E+04 | 2.03E−03 | 0.998 |
| CD3 × CD33-BspAb92 | 0.33 | 4.38E−08 | 1.16E+04 | 5.10E−04 | 0.9986 |
| CD3 × CD33-BspAb93 | 0.49 | 1.48E−08 | 3.49E+05 | 5.16E−03 | 0.9872 |
| CD3 × CD33-BspAb94 | 0.18 | 6.45E−07 | 1.15E+05 | 7.38E−02 | 0.9966 |
| CD3 × CD33-BspAb95 | 0.49 | 8.44E−09 | 5.42E+05 | 4.57E−03 | 0.966 |
| CD3 × CD33-BspAb96 | 0.11 | 5.73E−07 | 3.66E+05 | 2.10E−01 | 0.9823 |
| CD3 × CD33-BspAb97 | 0.32 | 2.76E−07 | 3.09E+05 | 8.54E−02 | 0.9991 |
| CD3 × CD33-BspAb98 | 0.45 | 5.10E−09 | 6.53E+05 | 3.33E−03 | 0.9554 |
| CD3 × CD33-BspAb99 | 0.01* | 3.07E−08 | 1.37E+05 | 4.21E−03 | 0.603* |
| CD3 × CD33-BspAb100 | 0.30 | 2.35E−07 | 3.54E+05 | 8.31E−02 | 0.974 |
| CD3 × CD33-BspAb101 | 0.02* | 3.40E−09 | 8.07E+05 | 2.75E−03 | 0.871* |
| CD3 × CD33-BspAb102 | 0.27 | 1.17E−07 | 4.59E+03 | 5.37E−04 | 1.000 |
| CD3 × CD33-BspAb103 | 0.43 | 3.52E−08 | 1.52E+04 | 5.37E−04 | 0.999 |
| CD3 × CD33-BspAb104 | 0.06 | 2.30E−08 | 1.25E+05 | 2.87E−03 | 0.872* |
| CD3 × CD33-BspAb105 | 0.22 | 5.17E−07 | 3.56E+05 | 1.84E−01 | 0.997 |
| CD3 × CD33-BspAb106 | 0.16 | 4.01E−07 | 4.68E+05 | 1.87E−01 | 0.944 |
| CD3 × CD33-BspAb107 | 0.04* | 1.05E−08 | 3.80E+05 | 4.00E−03 | 0.713* |
| CD3 × CD33-BspAb108 | 0.01* | 1.40E−06 | 2.94E+03 | 4.12E−03 | 0.391* |
| CD3 × CD33-BspAb109 | 0.01* | 1.32E−10 | 1.25E+04 | 1.65E−06 | 0.000* |
| CD3 × CD33-BspAb110 | 0.16 | 3.91E−07 | 3.95E+05 | 1.54E−01 | 0.968 |
| CD3 × CD33-BspAb111 | 0.29 | 2.93E−07 | 3.34E+05 | 9.79E−02 | 0.993 |
| CD3 × CD33-BspAb112 | 0.16 | 8.81E−07 | 3.79E+05 | 3.34E−01 | 0.996 |
| CD3 × CD33-BspAb113 | 0.35 | 2.25E−07 | 2.51E+05 | 5.64E−02 | 0.998 |
| CD3 × CD33-BspAb114 | 0.31 | 3.59E−07 | 3.25E+05 | 1.17E−01 | 0.996 |
| CD3 × CD33-BspAb115 | 0.55 | 1.62E−08 | 2.49E+05 | 4.02E−03 | 0.990 |
| CD3 × CD33-BspAb116 | 0.33 | 1.63E−07 | 1.03E+05 | 1.68E−02 | 0.997 |
| CD3 × CD33-BspAb117 | 0.42 | 1.66E−08 | 3.35E+05 | 5.54E−03 | 0.984 |
| CD3 × CD33-BspAb118 | 0.57 | 1.91E−09 | 4.03E+05 | 7.67E−04 | 0.989 |
| CD3 × CD33-BspAb119 | 0.48 | 2.17E−08 | 3.17E+05 | 6.88E−03 | 0.986 |
| CD3 × CD33-BspAb120 | 0.45 | 6.90E−08 | 2.74E+05 | 1.89E−02 | 0.985 |
| CD3 × CD33-BspAb121 | 0.01* | 1.21E−06 | 1.79E+04 | 2.16E−02 | 0.814* |
| CD3 × CD33-BspAb122 | 0.01* | <1.0E−12 | 1.54E+04 | <1.0E−07 | 0.557* |
| CD3 × CD33-BspAb123 | 0.22 | 1.20E−08 | 8.55E+04 | 1.03E−03 | 0.951 |
| CD3 × CD33-BspAb124 | 0.00* | <1.0E−12 | 1.25E+04 | <1.0E−07 | 0.000* |
| CD3 × CD33-BspAb125 | 0.30 | 3.19E−07 | 1.18E+04 | 3.75E−03 | 0.999 |
| CD3 × CD33-BspAb126 | 0.54 | 7.84E−08 | 1.49E+05 | 1.17E−02 | 0.995 |
| CD3εδ -specific | 0.04* | 2.30E−08 | 4.43E+05 | 1.02E−02 | 0.910 |
| monovalent control | | | | | |
| antibody | | | | | |
| CD3εδ -specific | 0.05 | 8.54E−10 | 1.25E+04 | 1.07E−05 | 0.000* |
| bivalent control | | | | | |
| antibody | | | | | |
| CD3εδ -specific | 0.02* | 5.07E−09 | 1.20E+06 | 6.09E−03 | 0.8167* |
| monovalent control | | | | | |
| antibody | | | | | |
| CD3εδ -specific | 0.05 | 2.71E−07 | 1.58E+05 | 4.30E−02 | 0.9152 |
| bivalent control | | | | | |
| antibody | | | | | |
| CD3 × CD33-BspAb44 | 0.54 | 4.15E−09 | 3.49E+05 | 1.45E−03 | 0.987 |
| CD3 × CD33-BspAb48 | 0.09 | 1.38E−07 | 1.85E+04 | 2.55E−03 | 0.992 |
| High affinity Control | 0.23 | 4.49E−08 | 2.48E+05 | 1.11E−02 | 0.995 |
| Low affinity Control | 0.05 | 4.38E−09 | 1.25E+04 | 5.48E−05 | 0.000* |
| High affinity Control | 0.23 | 3.95E−08 | 2.59E+05 | 1.02E−02 | 0.9943 |
| Low affinity Control | 0.04* | 4.27E−09 | 1.25E+04 | 5.33E−05 | 0* |
| | *low value | | | | *low value |

### Example 5

### Testing the Efficacy of Bispecific in Human Peripheral Blood Mononuclear Cells (PBMCS)

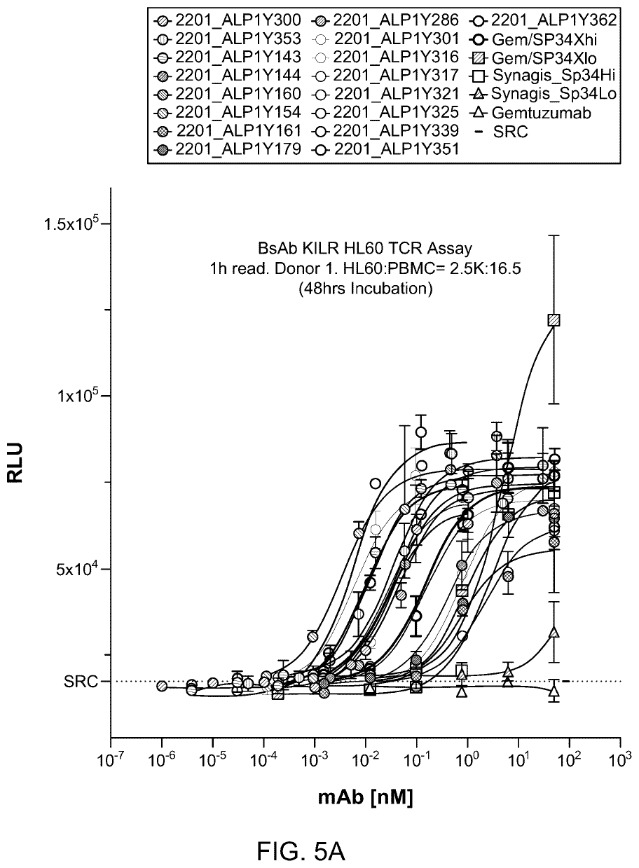

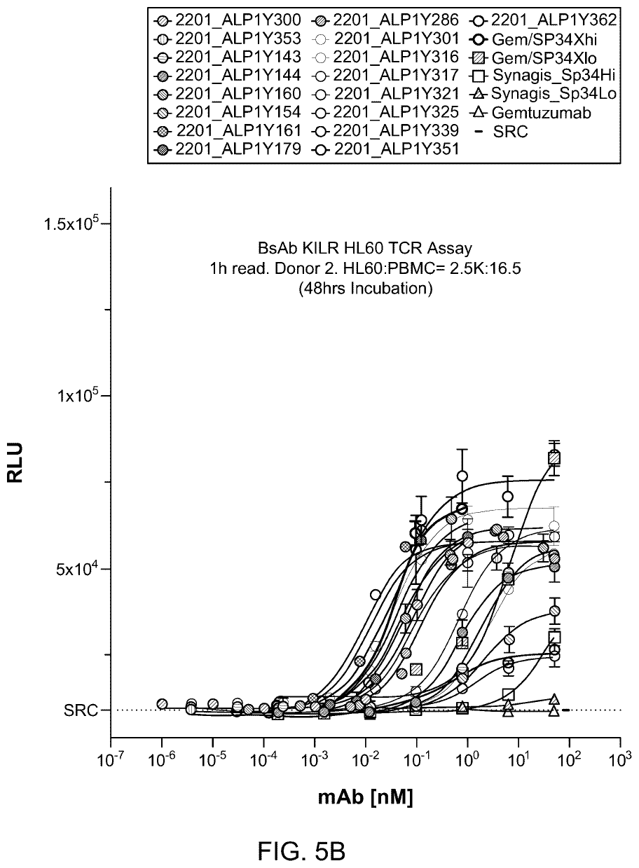

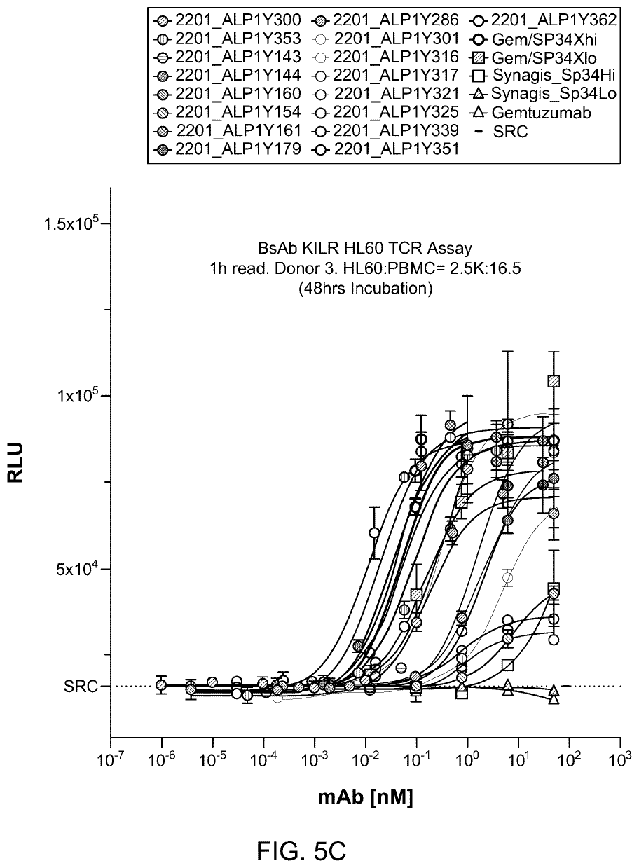

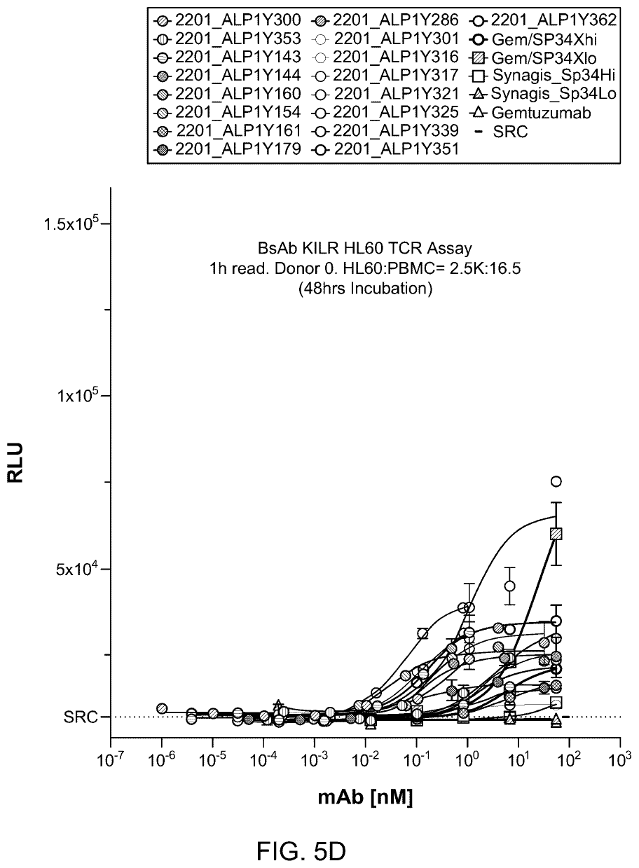

A panel of bispecific antibodies was selected for further characterization in additional assays to assess their functionality and toxicity. Killing Immune-Lysis Reaction (KILR) T Cell Redirection (TCR) assay (DiscoverX KILR HL-60 TCR Cytotoxic Assay) evaluated the ability of the bispecific anti-CD3 antibodies to mediate an interaction between effector (human Pan-T or donor PBMC) and target (CD33) expressing cells resulting in the release of reporter element as well as in cellular toxicity (See FIGS. 5A-5D, Table 13, infra).

To generate a panel of bispecific anti-CD3 antibodies with good cytotoxic capacity yet minimal cytokine induction, we tested the supernatants of the effector human Pan T or PBMC cells in quantitative flow cytometry assay (LEGENDplex™, Biolegend, Cat: 741036) against IL2 (Table 14, infra), TNFα (Table 15, infra), and IFNγ (Table 16, infra),

TABLE 13
RTCC Assay

| | Donor 1 | | | Donor 2 | | | Donor 3 | | | Donor 0 | | |
|---|---|---|---|---|---|---|---|---|---|---|---|---|
| | EC50 | gMFI | | EC50 | gMFI | | EC50 | gMFI | | EC50 | gMFI | |
| BiSAb ID | (nM) | max | R² | (nM) | max | R² | (nM) | max | R² | (nM) | max | R² |
| CD3 × CD33-BspAb71 | 0.027 | 63010 | 0.98 | 0.083 | 57492 | 0.99 | 0.377 | 78563 | 1.00 | 0.479 | 23856 | 0.97 |
| CD3 × CD33-BspAb116 | 0.045 | 65975 | 0.93 | 0.121 | 52850 | 0.98 | 0.171 | 65799 | 0.98 | 0.126 | 24526 | 0.99 |
| CD3 × CD33-BspAb37 | 0.010 | 70563 | 0.98 | 0.042 | 59221 | 0.97 | 0.053 | 82310 | 0.95 | 0.172 | 31557 | 1.00 |
| CD3 × CD33-BspAb38 | 0.413 | 64778 | 1.00 | 0.717 | 50523 | 1.00 | 1.723 | 75839 | 1.00 | 1.488 | 23336 | 0.93 |
| CD3 × CD33-BspAb45 | 0.004 | 76051 | 0.97 | 0.012 | 55929 | 0.97 | 0.020 | 86741 | 0.99 | 0.030 | 23283 | 0.97 |
| CD3 × CD33-BspAb51 | 0.036 | 66855 | 0.97 | 0.050 | 55830 | 0.97 | 0.085 | 80586 | 0.97 | 0.200 | 28440 | 0.98 |
| CD3 × CD33-BspAb52 | 0.762 | 57768 | 0.98 | 2.281 | 37739 | 0.99 | 7.832 | 42697 | 1.00 | 5.208 | 16216 | 0.99 |
| CD3 × CD33-BspAb66 | 0.937 | 65809 | 0.98 | 1.846 | 53919 | 0.99 | 2.333 | 75941 | 0.96 | 2.916 | 29714 | 0.99 |
| CD3 × CD33-BspAb73 | 0.035 | 79871 | 0.97 | 0.089 | 56118 | 0.99 | 0.159 | 73957 | 0.99 | 0.086 | 15614 | 0.99 |
| CD3 × CD33-BspAb72 | 0.819 | 78068 | 0.99 | 2.430 | 54095 | 1.00 | 4.903 | 66216 | 1.00 | 5.363 | 21018 | 1.00 |
| CD3 × CD33-BspAb85 | 0.006 | 78176 | 0.97 | 0.028 | 64222 | 0.98 | 0.045 | 85566 | 0.95 | 0.102 | 29905 | 1.00 |
| CD3 × CD33-BspAb86 | 0.151 | 67276 | 0.98 | 0.697 | 59133 | 1.00 | 1.578 | 83470 | 0.95 | 1.707 | 24060 | 0.99 |
| CD3 × CD33-BspAb90 | 0.012 | 76918 | 0.98 | 0.034 | 62305 | 0.94 | 0.047 | 83956 | 0.98 | 0.147 | 34770 | 1.00 |
| CD3 × CD33-BspAb94 | 2.253 | 61742 | 0.99 | 1.357 | 24867 | 0.99 | 0.945 | 29374 | 0.96 | 1.325 | 15399 | 0.99 |
| CD3 × CD33-BspAb105 | 0.153 | 62652 | 0.97 | 0.507 | 26713 | 0.98 | 0.821 | 35049 | 0.99 | 0.478 | 10774 | 0.95 |
| CD3 × CD33-BspAb115 | 0.006 | 63020 | 0.95 | 0.009 | 51930 | 0.96 | 0.011 | 82682 | 0.98 | 0.054 | 38738 | 1.00 |
| CD3 × CD33-BspAb125 | 0.027 | 65480 | 0.98 | 0.049 | 54558 | 0.96 | 0.055 | 84180 | 0.96 | 0.166 | 26717 | 0.99 |
| High affinity Control | 0.040 | 81658 | 0.99 | 0.039 | 82922 | 0.98 | 0.031 | 87241 | 0.98 | 0.908 | 75062 | 0.91 |
| low affinity Control | 5.021 | 122021 | 0.99 | 6.677 | 82075 | 0.96 | 0.299 | 104125 | 0.97 | 23.710 | 59726 | 0.99 |
| hIgG1 Synagis_Sp34Hi | 2.773 | 71986 | 0.97 | 44.520 | 30241 | 1.00 | 46.210 | 44073 | 0.99 | ND | 11360 | 1.00 |
| hIgG1 Synagis_Sp34Lo | ~9168005365107 | 31677 | 0.94 | ND | ND | ND | ND | ND | ND | ND | ND | ND |
| Gemtuzumab | ND | ND | ND | ND | ND | ND | ND | ND | ND | ND | ND | ND |

TABLE 14
Cytokine Release Assay (IFNγ)

| | Donor 1 | | | Donor 2 | | | Donor 3 |
|---|---|---|---|---|---|---|---|
| BiSAb ID | EC50 (nM) | gMFI max | R² | EC50 (nM) | gMFI max | R² | EC50 (nM) |
| CD3 × CD33-BspAb71 | ND | 5378 | ND | 1.91 | 27703 | 1.00 | ~3712503736223 |
| CD3 × CD33-BspAb116 | ND | 5443 | ND | 0.1771 | 14072 | 0.95 | 1.511 |
| CD3 × CD33-BspAb37 | ND | 5562 | ND | 0.1402 | 32861 | 0.99 | 1.763 |
| CD3 × CD33-BspAb38 | ND | 4321 | ND | 13.36 | 17985 | 1.00 | 6.382 |
| CD3 × CD33-BspAb45 | ND | 4634 | ND | 0.06837 | 26909 | 0.91 | ND |
| CD3 × CD33-BspAb51 | 15.26 | 10833 | 0.99 | 0.2923 | 30586 | 0.99 | 0.214 |
| CD3 × CD33-BspAb52 | ND | 4225 | ND | ND | 4666 | ND | 25.37 |
| CD3 × CD33-BspAb66 | ND | 7652 | 0.96 | 22.89 | 28273 | 1.00 | 52.59 |
| CD3 × CD33-BspAb73 | ND | 4347 | ND | 8.614 | 26901 | 0.99 | 4.637 |
| CD3 × CD33-BspAb72 | ND | 4821 | ND | 49.06 | 12175 | 0.99 | ND |
| CD3 × CD33-BspAb85 | ND | 8042 | ND | 0.04678 | 31370 | 0.92 | ND |
| CD3 × CD33-BspAb86 | ND | 4691 | ND | 2.678 | 18489 | 0.99 | 7.929 |
| CD3 × CD33-BspAb90 | ND | 10814 | ND | 0.1262 | 31640 | 0.97 | 0.0851 |
| CD3 × CD33-BspAb94 | ND | 4553 | ND | ND | 4655 | ND | ND |
| CD3 × CD33-BspAb105 | ND | 4562 | ND | ND | 5334 | ND | ND |
| CD3 × CD33-BspAb115 | 0.1911 | 45118 | 0.99 | 0.03964 | 66767 | 0.86 | 0.04025 |
| CD3 × CD33-BspAb125 | ND | 4936 | ND | 0.1028 | 16190 | 0.99 | 0.06305 |
| High affinity Control | 6.993 | 911706 | 1.00 | 2.164 | 1413786 | 0.93 | 0.1256 |
| Low affinity Control | 14.36 | 129186 | 1.00 | 16.41 | 712053 | 1.00 | 11.43 |
| hIgG1 Synagis_Sp34Hi | ND | 4789 | ND | 77.7 | 62571 | 1.00 | ND |
| hIgG1 Synagis_Sp34Lo | ND | 4636 | ND | ND | 5613 | ND | ND |
| Gemtuzumab | ND | 4629 | ND | ND | 3585 | ND | ND |

| | Donor 3 | | Donor 0 | | |
|---|---|---|---|---|---|
| BiSAb ID | gMFI max | R² | EC50 (nM) | gMFI max | R² |
| CD3 × CD33-BspAb71 | 331871 | 1.00 | ND | 812403 | ND |
| CD3 × CD33-BspAb116 | 166770 | 0.99 | ND | 186959 | ND |
| CD3 × CD33-BspAb37 | 316530 | 1.00 | 0.252 | 1056106 | 1.00 |
| CD3 × CD33-BspAb38 | 98493 | 0.99 | 8.377 | 555498 | 0.99 |
| CD3 × CD33-BspAb45 | 289748 | ND | 0.09598 | 1388924 | 0.93 |
| CD3 × CD33-BspAb51 | 355475 | 0.91 | 0.2693 | 1340451 | 0.94 |
| CD3 × CD33-BspAb52 | 8065 | 0.98 | ND | 6346 | ND |
| CD3 × CD33-BspAb66 | 378966 | 1.00 | 12.88 | 1250982 | 1.00 |
| CD3 × CD33-BspAb73 | 197820 | 0.99 | ND | 308014 | ND |
| CD3 × CD33-BspAb72 | 82790 | ND | ND | 465449 | ND |
| CD3 × CD33-BspAb85 | 104416 | ND | 0.08319 | 983149 | 0.97 |
| CD3 × CD33-BspAb86 | 152316 | 0.99 | 2.346 | 573346 | 0.89 |
| CD3 × CD33-BspAb90 | 512855 | 0.89 | 0.1913 | 1705521 | 0.96 |
| CD3 × CD33-BspAb94 | 4151 | ND | ND | 4667 | ND |
| CD3 × CD33-BspAb105 | 5115 | ND | ND | 11319 | ND |
| CD3 × CD33-BspAb115 | 475087 | 0.89 | 0.04182 | 1599339 | 0.88 |
| CD3 × CD33-BspAb125 | 169586 | 0.94 | ~12.29 | 957996 | 1.00 |
| High affinity Control | 1678765 | 0.96 | 0.04496 | 2816615 | 0.97 |
| Low affinity Control | 1084219 | 1.00 | 0.3081 | 2922778 | 0.98 |
| hIgG1 Synagis_Sp34Hi | 35105 | ND | ND | 148048 | ND |
| hIgG1 Synagis_Sp34Lo | 4070 | ND | ND | 4606 | ND |
| Gemtuzumab | 4481 | ND | ND | 5339 | ND |

TABLE 15
Cytokine Release Assay (TNFα)

| | Donor 1 | | | Donor 2 | | | Donor 3 | | | Donor 0 | | |
|---|---|---|---|---|---|---|---|---|---|---|---|---|
| | EC50 | gMFI | | EC50 | gMFI | | EC50 | gMFI | | EC50 | gMFI | |
| BiSAb ID | (nM) | max | R² | (nM) | max | R² | (nM) | max | R² | (nM) | max | R² |
| CD3 × CD33-BspAb71 | ND | 5473 | ND | 0.3958 | 16716 | ND | ND | 28222 | ND | ND | 44063 | ND |
| CD3 × CD33-BspAb116 | ND | 4927 | ND | 0.1547 | 9078 | 0.94 | 0.6849 | 13575 | 0.94 | 0.6098 | 17966 | 0.98 |
| CD3 × CD33-BspAb37 | ND | 5608 | ND | 0.08322 | 15827 | 0.99 | 0.2221 | 31631 | 1.00 | 0.1645 | 48344 | 0.99 |
| CD3 × CD33-BspAb38 | ND | 4836 | ND | 4.029 | 10061 | 0.99 | 5.948 | 17928 | 0.99 | 6.014 | 28500 | 0.99 |
| CD3 × CD33-BspAb45 | ND | 4854 | ND | 0.03339 | 12115 | 0.89 | 0.02163 | 31777 | 0.88 | 0.09192 | 71341 | 0.97 |
| CD3 × CD33-BspAb51 | 0.5349 | 6203 | 0.90 | 0.1804 | 14790 | 0.96 | 0.1664 | 36152 | 0.94 | 0.2028 | 51709 | 0.95 |
| CD3 × CD33-BspAb52 | ND | 4512 | ND | 1.347 | 4796 | 0.84 | 18.65 | 5311 | 0.90 | ND | 5506 | ND |
| CD3 × CD33-BspAb66 | ND | 5358 | ND | 28.13 | 13976 | 0.99 | 10.8 | 28579 | 0.99 | 20.09 | 55773 | 1.00 |
| CD3 × CD33-BspAb73 | ND | 4395 | ND | 1.012 | 14288 | 0.98 | 0.6526 | 24220 | 0.99 | 0.7594 | 28038 | 0.89 |
| CD3 × CD33-BspAb72 | ND | 4412 | ND | 16.91 | 8012 | 0.99 | 42.55 | 11154 | 1.00 | ND | 23361 | 1.00 |
| CD3 × CD33-BspAb85 | 0.6467 | 5487 | 0.88 | 0.04972 | 18397 | 0.95 | 0.06351 | 26600 | 0.94 | 0.114 | 44799 | 0.98 |
| CD3 × CD33-BspAb86 | ND | 4900 | ND | 2.189 | 10882 | 0.98 | 5.578 | 25170 | 0.99 | 2.153 | 34071 | 0.87 |
| CD3 × CD33-BspAb90 | ND | 5326 | ND | 0.07882 | 15969 | 0.99 | 0.07242 | 32823 | 0.97 | 0.136 | 61677 | 0.95 |
| CD3 × CD33-BspAb94 | ND | 4672 | ND | ND | 4423 | ND | ND | 4108 | ND | ND | 5151 | ND |
| CD3 × CD33-BspAb105 | ND | 4473 | ND | ND | 5503 | ND | ND | 4391 | ND | ND | 5208 | ND |
| CD3 × CD33-BspAb115 | 0.05533 | 12972 | 0.95 | 0.04515 | 24092 | 0.91 | 0.04599 | 48556 | 0.96 | 0.08144 | 91597 | 0.98 |
| CD3 × CD33-BspAb125 | ND | 5098 | ND | 0.054 | 11970 | 0.96 | 0.04462 | 17759 | 0.88 | 0.4753 | 39618 | 1.00 |
| High affinity Control | 13.14 | 113158 | 1.00 | 5.976 | 124569 | 0.92 | 0.212 | 129075 | 0.96 | 0.4592 | 231030 | 0.99 |
| Low affinity Control | ND | 82783 | ND | 24.56 | 134766 | 1.00 | 40.69 | 122386 | 1.00 | 0.5495 | 222368 | 0.95 |
| hIgG1 Synagis_Sp34Hi | ND | 4912 | ND | 26.04 | 21673 | 1.00 | ND | 6853 | ND | ND | 18150 | 0.98 |
| hIgG1 Synagis_Sp34Lo | ND | 4642 | ND | 28.82 | 5718 | 0.95 | ND | 3726 | ND | ND | 4772 | ND |
| Gemtuzumab | ND | 4426 | ND | ND | 3679 | ND | ND | 3960 | ND | ND | 5312 | ND |

TABLE 16
Cytokine Release Assay (IL2)

| | Donor 1 | | | Donor 2 | | | Donor 3 | | | Donor 0 | | |
|---|---|---|---|---|---|---|---|---|---|---|---|---|
| | EC50 | gMFI | | EC50 | gMFI | | EC50 | gMFI | | EC50 | gMFI | |
| BiSAb ID | (nM) | max | R² | (nM) | max | R² | (nM) | max | R² | (nM) | max | R² |
| CD3 × CD33-BspAb71 | ND | 24419 | 0.99 | 0.07245 | 30872 | 0.99 | 2.16 | 126591 | 1.00 | 3.03 | 65465 | 1.00 |
| CD3 × CD33-BspAb116 | 0.3841 | 18977 | 0.98 | 0.09089 | 16763 | 0.86 | 0.198 | 27240 | 0.93 | 0.4501 | 23226 | 0.99 |
| CD3 × CD33-BspAb37 | 0.4372 | 26794 | 1 | 0.1408 | 34788 | 0.98 | 0.1809 | 104035 | 1.00 | 0.18 | 69244 | 0.99 |
| CD3 × CD33-BspAb38 | 2.869 | 16171 | 0.94 | 1.73 | 19186 | 0.99 | 1.838 | 45880 | 1.00 | 6.626 | 39254 | 0.99 |
| CD3 × CD33-BspAb45 | 0.08694 | 22155 | 0.97 | 0.02039 | 26215 | 0.96 | 0.02047 | 67854 | 0.95 | 0.05395 | 50916 | 0.87 |
| CD3 × CD33-BspAb51 | 0.529 | 37819 | 0.97 | 0.2052 | 27287 | 0.94 | 0.09949 | 155832 | 0.90 | 0.1808 | 104557 | 0.97 |
| CD3 × CD33-BspAb52 | 10.71 | 14497 | 0.92 | ND | 12791 | ND | 3.865 | 18063 | 0.94 | 140.7 | 17174 | 0.93 |
| CD3 × CD33-BspAb66 | 40.1 | 31148 | 1 | 38.74 | 33694 | 1.00 | 49.75 | 94973 | 1.00 | 44.86 | 84690 | 1.00 |
| CD3 × CD33-BspAb73 | 0.3728 | 15943 | ND | 26.46 | 32508 | 0.96 | 0.2396 | 42921 | 0.98 | 0.104 | 28977 | 0.95 |
| CD3 × CD33-BspAb72 | 4.284 | 17331 | 0.98 | 2.157 | 16394 | 0.93 | 8.355 | 26269 | 1.00 | 8.213 | 22977 | 0.99 |
| CD3 × CD33-BspAb85 | 0.4415 | 23141 | 0.99 | 0.2215 | 32629 | 0.99 | 0.09259 | 49715 | 0.99 | 0.1208 | 32986 | 0.98 |
| CD3 × CD33-BspAb86 | 4.237 | 18817 | 0.97 | 6.459 | 22130 | 0.98 | 3.523 | 42106 | 1.00 | 4.13 | 34392 | 0.98 |
| CD3 × CD33-BspAb90 | 0.08322 | 24330 | 0.91 | 0.06124 | 30007 | 0.98 | ND | 79297 | 0.81 | 0.08872 | 69647 | 0.97 |
| CD3 × CD33-BspAb94 | 3.58 | 14811 | 0.96 | ND | 12479 | ND | 1.472 | 13878 | 0.93 | ND | 13559 | ND |
| CD3 × CD33-BspAb105 | 0.2316 | 13384 | 0.09 | ND | 15857 | ND | 0.1407 | 17955 | 0.91 | 0.4544 | 16915 | 0.94 |
| CD3 × CD33-BspAb115 | 0.07554 | 99289 | 0.97 | 0.08065 | 45235 | 0.97 | 0.04793 | 300436 | 0.96 | 0.2056 | 242139 | 1.00 |
| CD3 × CD33-BspAb125 | 0.1017 | 22318 | 0.98 | 0.1151 | 25090 | 0.96 | ND | 65843 | ND | 0.1055 | 52780 | 0.99 |
| High affinity Control | 1.639 | 179041 | 0.99 | 16.66 | 121009 | 0.96 | 0.1715 | 346977 | 0.99 | 0.4559 | 376528 | 0.98 |
| Low affinity Control | 10.76 | 18465 | 0.99 | ND | 15713 | ND | 2.921 | 21164 | 0.95 | 2.286 | 24343 | 0.98 |
| hIgG1 Synagis_Sp34Hi | ND | 15385 | 0.92 | 29.77 | 81190 | 1.00 | 22.8 | 25490 | 0.99 | ND | 39677 | 1.00 |
| hIgG1 Synagis_Sp34Lo | ND | 12643 | ND | ND | 16145 | 0.94 | ND | 14041 | ND | 0.6993 | 12980 | ND |
| Gemtuzumab | ND | 12947 | ND | ND | 10696 | ND | ND | 12203 | ND | 5.36 | 12483 | ND |

### Example 6

### Characterization of Favorable Biophysical Characteristics

Tₘ/T_{agg} Analysis

Samples were loaded in duplicate into 9 μL wells called a Uni on an UNCLE instrument (Unchained Labs) and thermal data were analyzed by the Uncle instrument software. The following parameters were obtained: (1) starting temperature of first melt (Tₒₙₛₑₜ), (2) melting temperature transitions (T_{M}), and (3) aggregation temperature transitions at two different wavelengths (T_{agg}). Full spectrum intrinsic fluorescence was used to obtain Tₘ and Tₒₙₛₑₜ while static light scattering was used to obtain T_{agg}.

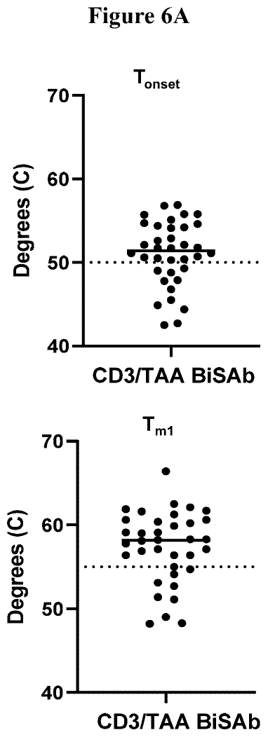

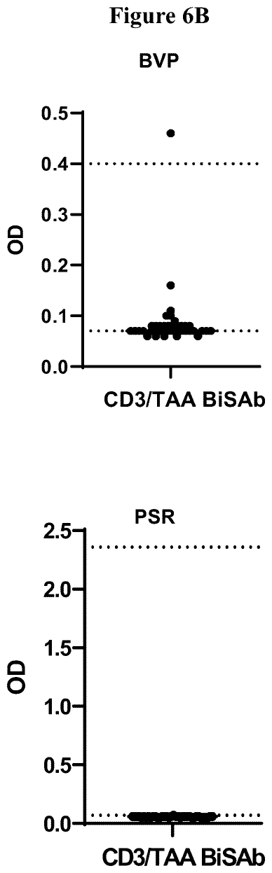

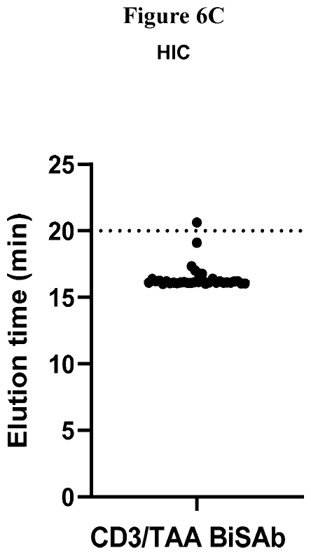

Polyspecific Reagent/nonspecific binding (PSR/NSB) ELISA PSR/NSB ELISA was employed to measure polyspecific/nonspecific antibody binding to various antigens: ds DNA (D4544, Sigma) and ss DNA (D8899 Sigma), cardiolipin (C0563, Sigma), insulin (19278, Sigma), lipopolysaccharide (LPS; tlrl-eblps, InvivoGen), and Hemocyanin (H8283, Sigma). The wells were coated with a mixture of the antigens, and the plate was then blocked with 1% BSA. Wells were washed after every step, test antibodies and controls were added followed by a secondary RP-conjugated Goat anti-human IgG Fc (Jackson ImmunoReasearch Labs, Inc., West Grove, PA). The plate was developed with TMB and read on a plate reader with the setting at OD650. Results are described in FIG. 6A and Table 17.

### Baculovirus Particles (BVP) ELISA

BVP ELISA was used to measure nonspecific/polyspecific antibody binding to Baculovirus Particles (BVP). The plate was coated with BVP antigen (E3001 Medna), and the plate was blocked with 1% BSA, washes followed every step, test antibodies and controls were added followed by a secondary RP-conjugated Goat anti-human IgG Fc (Jackson ImmunoReasearch Labs, Inc., West Grove, PA). The plate was developed with TMB and read on a plate reader with the setting at OD₆₅₀. Results are described in FIG. 6B and Table 17.

### Hydrophobic Interaction Chromatography (HIC) Analysis

Each bispecific Ab was diluted with HN₄SO₄ to final concentration of 1 M HN₄SO₄, and 10 ug was injected into HPLC. The antibody species were then eluted using a reverse NaCl gradient (high concentration to low concentration). The retention time of the antibody was measured on a HIC-butyl column (Proteomix HIC Butyl-NP5) with less hydrophobic species eluting earlier and more hydrophobic species eluting later. Results are described in FIG. 6C and Table 17.

TABLE 17
Developability assessments of CD3 Bispecific molecules

| | Average | Average | Average | Average | Average | HIC | BVP | PSR |
|---|---|---|---|---|---|---|---|---|
| | Tonset | Tm1 | Tm2 | Tagg 266 | Tagg 473 | Elution Time | ELISA | ELISA |
| Sample ID | (° C.) | (° C.) | (° C.) | (° C.) | (° C.) | (min) | (OD) | (OD) |
| CD3 × CD33-BspAb68 | 45.5 | 51.1 | ND | 72.9 | 73.2 | 16.12 | 0.08 | 0.06 |
| CD3 × CD33-BspAb71 | 50.7 | 56.4 | ND | 73.1 | 73.4 | 16.09 | 0.07 | 0.06 |
| CD3 × CD33-BspAb72 | 55.8 | 62.1 | 72.2 | 75.1 | 75.6 | 16.15 | 0.1 | 0.06 |
| CD3 × CD33-BspAb73 | 42.5 | 54.1 | 72.3 | 74.4 | 75 | 16.02 | 0.08 | 0.05 |
| CD3 × CD33-BspAb77 | 50.3 | 54.7 | ND | 73.4 | 73.9 | 16.41 | 0.07 | 0.06 |
| CD3 × CD33-BspAb78 | 52.6 | 57.8 | 72 | 73.9 | 74.2 | 16.11 | 0.07 | 0.06 |
| CD3 × CD33-BspAb79 | 48.8 | 52.7 | 66.5 | 74.1 | 74.3 | 16.12 | 0.07 | 0.06 |
| CD3 × CD33-BspAb80 | 49.3 | 56.4 | ND | 75 | 75.4 | 16.09 | 0.06 | 0.05 |
| CD3 × CD33-BspAb81 | 51.7 | 58.3 | ND | 74.2 | 74.3 | 16.14 | 0.07 | 0.06 |
| CD3 × CD33-BspAb84 | 55.7 | 60.6 | 71.5 | 74.2 | 74.4 | 16.05 | 0.06 | 0.06 |
| CD3 × CD33-BspAb85 | 54.1 | 59.9 | ND | 73.8 | 74.2 | 16.25 | 0.06 | 0.06 |
| CD3 × CD33-BspAb86 | 52.1 | 59.1 | ND | 73.9 | 74.2 | 19.12 | 0.08 | 0.06 |
| CD3 × CD33-BspAb90 | 56.9 | 62.5 | 71.2 | 74 | 74.4 | 16.03 | 0.08 | 0.06 |
| CD3 × CD33-BspAb91 | 47.9 | 58.1 | 72 | 73.9 | 74.1 | 16.78 | 0.07 | 0.05 |
| CD3 × CD33-BspAb92 | 51.7 | 59.1 | 72.2 | 74.1 | 74.3 | ND | 0.06 | 0.05 |
| CD3 × CD33-BspAb93 | 42.7 | 48.3 | 65.7 | 73.7 | 74.2 | 16.12 | 0.16 | 0.05 |
| CD3 × CD33-BspAb94 | 44.9 | 49 | ND | 74.3 | 74.8 | 17.03 | 0.08 | 0.06 |
| CD3 × CD33-BspAb95 | 47.8 | 53.1 | ND | 73.3 | 73.7 | 16.12 | 0.07 | 0.06 |
| CD3 × CD33-BspAb98 | 46.8 | 51.4 | ND | 73.8 | 74.1 | 16.19 | 0.07 | 0.06 |
| CD3 × CD33-BspAb105 | 55.8 | 61.7 | ND | 74.1 | 74.6 | 16.14 | 0.07 | 0.05 |
| CD3 × CD33-BspAb115 | 51.1 | 57.1 | 72.4 | 74.3 | 74.8 | 16.15 | 0.07 | 0.06 |
| CD3 × CD33-BspAb116 | 52.9 | 58.3 | 72.2 | 74.6 | 75.2 | 16.14 | 0.07 | 0.05 |
| CD3 × CD33-BspAb117 | 44.4 | 48.2 | 59.6 | 74.1 | 74.3 | 16.22 | 0.08 | 0.05 |
| CD3 × CD33-BspAb118 | 49 | 55 | ND | 73. | 74.2 | 16.35 | 0.08 | 0.05 |
| CD3 × CD33-BspAb119 | 54.4 | 60.4 | ND | 73.2 | 73.7 | 16.08 | 0.1 | 0.06 |
| CD3 × CD33-BspAb124 | 56.8 | 66.4 | 86.7 | 73.2 | 73.9 | 16.05 | 0.08 | 0.06 |
| CD3 × CD33-BspAb126 | 54.2 | 60.2 | 70.5 | 73.5 | 73.8 | 16.07 | 0.09 | 0.06 |
| CD3 × CD33-BspAb42 | 50.6 | 56.4 | 72.7 | 74.4 | 74.7 | 16.38 | 0.06 | 0.05 |
| CD3 × CD33-BspAb38 | 50.5 | 59 | 72 | 74.1 | 74.5 | 19.65, 20.64 | 0.08 | 0.06 |
| CD3 × CD33-BspAb52 | 51.1 | 58.2 | 72.1 | 74 | 74.4 | ND | 0.46 | 0.07 |
| CD3 × CD33-BspAb45 | 51.8 | 56.9 | 72 | 74.1 | 74.6 | 17.35 | 0.08 | 0.05 |
| CD3 × CD33-BspAb44 | 50.4 | 57.1 | ND | 73.7 | 74.1 | 16.21 | 0.07 | 0.05 |
| CD3 × CD33-BspAb65 | 55.1 | 60.6 | 73.2 | 75.5 | 76.2 | 16.83 | 0.07 | 0.06 |
| CD3 × CD33-BspAb36 | 54.6 | 61.6 | 71.7 | 73.4 | 73.8 | 16.22 | 0.11 | 0.06 |
| CD3 × CD33-BspAb37 | 54.7 | 61.9 | 82.8 | 73.6 | 73.9 | 16.22 | 0.07 | 0.06 |
| CD3 × CD33-BspAb51 | 52.11 | 61.26 | 69.9 | 73.3 | 73.63 | 16.16 | 0.07 | 0.05 |

What is claimed is:

1. An isolated anti-CD3 antibody, or an antigen-binding fragment thereof, comprising:
i) a heavy chain variable region comprising a VHCDR1 selected from any of SEQ ID NOs: 345-457, a VHCDR2 selected from any of SEQ ID NOs: 458-573, and a VHCDR3 selected from any of SEQ ID NOs: 574-687 and
ii) a light chain variable region comprising a VLCDR1 selected from any of SEQ ID NOs:1-113, a VLCDR2 selected from any of SEQ ID NOs: 114-226, and a VLCDR3 selected from any of SEQ ID NOs:227-377.

2. The antibody, or antigen-binding fragment thereof, of claim 1, wherein the VH is selected from any one of SEQ ID NOs: 915-1141.

3. The antibody, or antigen-binding fragment thereof, of claim 1 or claim 2, wherein the VL is selected from any one of SEQ ID NOs: 688-914.

4. The antibody, or antigen-binding fragment thereof, of any one of claims 1-3, wherein the antibody is human.

5. The antibody, or antigen-binding fragment thereof, of any one of claims 1-3, wherein the antibody is chimeric.

6. The antibody, or antigen-binding fragment thereof, of any one of claims 1-5, wherein the antibody is selected from a single-variable domain antibody, single chain antibody, a scFv, a bispecific antibody, a multi-specific antibody, a Fab, a F(ab′)2, and a whole antibody.

7. The antibody, or antigen-binding fragment thereof, of claim 6, wherein the antibody is a multi-specific antibody.

8. The antibody, or antigen-binding fragment thereof, of claim 6, wherein the antibody is a bispecific antibody comprising first and second Fc domains.

9. The antibody, or antigen-binding fragment thereof, of claim 8, wherein the bispecific antibody further comprises a targeting antibody.

10. The antibody, or antigen-binding fragment thereof, of claim 9, wherein the targeting antibody is an anti-tumor antibody, or an antigen-binding fragment thereof.

11. The antibody, or antigen-binding fragment thereof, of any one of claims 6-10, wherein the anti-CD3 antibody, or the antigen-binding fragment thereof, comprises an scFv.

12. The antibody, or antigen-binding fragment thereof, of claim 6 or claim 11, wherein the anti-CD3 scFv antibody comprises an amino acid sequence selected from any one of SEQ ID NOs: 1155-1236.

13. The antibody, or antigen-binding fragment thereof, of claim 11 or 12, wherein the anti-CD3 scFv antibody is operably linked to the first Fc domain.

14. The antibody, or antigen-binding fragment thereof, of claim 13, wherein the anti-CD3 scFv antibody is operably linked to the first Fc domain through a first linker.

15. The antibody, or antigen-binding fragment thereof, of any one of claims 9-14, wherein the targeting antibody, or the antigen-binding fragment thereof, comprises a Fab fragment or a F(ab′)₂ fragment.

16. The antibody, or antigen-binding fragment thereof, of claim 15, wherein the targeting Fab or F(ab′)₂ fragment is operably linked to the second Fc domain.

17. The antibody, or antigen-binding fragment thereof, of claim 16, wherein the targeting Fab or F(ab′)₂ fragment is operably linked to the second Fc domain through a second linker.

18. The antibody, or antigen-binding fragment thereof, of claim 15 or 17, wherein the first linker or second linker is a variable length Gly-Ser linker.

19. The antibody, or antigen-binding fragment thereof, of claim 18, wherein the first linker and second linker are independently selected from SEQ ID NOs.: 1146-1149, and 1237-1238.

20. The antibody, or antigen-binding fragment thereof, of any one of claims 8-19, wherein the first and second Fc domain comprises complementary mutations selected from knobs-into-holes (KIH) mutations, K409R/F405L, HA-TF and related mutations, skew mutations, ZW mutations, 7.8.60 mutations or related mutations, DD-KK mutations or related mutations, EW-RVT mutations or related mutations, strand-exchange engineered domains (SEED), Duobody mutations, CrossMAb mutations, and A107 mutations or related mutations.

21. The antibody, or antigen-binding fragment thereof, of claim 20, wherein (1) the first Fc domain comprises a T366W mutation and the second Fc domain comprises T366S/L368A/Y407V mutations; (2) the first Fc domain comprises T366S/L368A/Y407V mutations and the second Fc domain comprises a T366W mutation; (3) the first Fc domain comprises S354C/T366W mutations and the second Fc domain comprises Y349C/T366S/L368A/Y407V mutations; (4) the first Fc domain comprises Y349C/T366S/L368A/Y407V mutations and the second Fc domain comprises S354C/T366W mutation; (5) the first Fc domain comprises S354C/T366W mutations and the second Fc domain comprises Y349C/T366S/L368A/Y407V/H435R/Y436F mutations; (6) the first Fc domain comprises Y349C/T366S/L368A/Y407V/H435R/Y436F mutations and the second Fc domain comprises S354C/T366W mutations; 7) the first Fc domain comprises S354C/T366W/H435R/Y436F mutations and the second Fc domain comprises Y349C/T366S/L368A/Y407V mutations; or 8) the first Fc domain comprises Y349C/T366S/L368A/Y407V mutations and the second Fc domain comprises S354C/T366W/H435R/Y436F mutations.

22. The antibody, or antigen-binding fragment thereof, of claim 20, wherein (1) the first and second Fe domains comprise the amino acid sequences of SEQ ID NOs: 1142 and 1143, respectively; or (2) the first and second Fe domains comprise the amino acid sequences of SEQ ID NOs: 1143 and 1142, respectively.

23. The antibody, or antigen-binding fragment thereof, of claim 20, wherein (1) the anti-CD3 antibody comprises a constant region comprising the amino acid sequence of SEQ ID NO: 1144, and the anti-tumor antibody comprises a constant region comprising the amino acid sequence of SEQ ID NO: 1145; or (2) the anti-CD3 antibody comprises a constant region comprising the amino acid sequence of SEQ ID NO: 1145, and the anti-tumor antibody comprises a constant region comprising the amino acid sequence of SEQ ID NO: 1144.

24. The antibody, or antigen-binding fragment thereof, of claim 20, wherein (1) the first Fc domain comprises a S364H mutation and the second Fc domain comprises a Y349T mutation; (2) the first Fc domain comprises a Y349T mutation and the second Fc domain comprises a S364H mutation; (3) the first Fc domain comprises a F405A mutation and the second Fc domain comprises a T394F mutation; (4) the first Fc domain comprises a T394F mutation and the second Fc domain comprises a F405A mutation; (5) the first Fc domain comprises S364H/T394F mutations and the second Fc domain comprises Y349T/F405A mutations; (6) the first Fc domain comprises Y349T/F405A mutations and the second Fc domain comprises S364H/T394F mutations; (7) the first Fc domain comprises S364H/F405A mutations and the second Fc domain Y349T/T394F mutations; or (8) the first Fc domain comprises Y349T/T394F mutations and the second Fc domain S364H/F405A mutations.

25. The antibody, or antigen-binding fragment thereof, of claim 20, wherein (1) the first Fc domain comprises S364K/E357Q mutations and the second Fc domain comprises L368D/K370S mutations; (2) the first Fc domain comprises L368D/K370S mutations and the second Fc domain comprises S364K/E357Q mutations; (3) the first Fc domain comprises L368D/K370S mutations and the second Fc domain comprises a S364K mutation; (4) the first Fc domain comprises a S364K mutation and the second Fc domain comprises L368D/K370S mutations; (5) the first Fc domain comprises L368E/K370S mutations and the second Fc domain comprises a S364K mutation; (6) the first Fc domain comprises a S364K mutation and the second Fc domain comprises L368E/K370S mutations; (7) the first Fc domain comprises T411E/K360E/Q362E mutations and the second Fc domain comprises a D401K mutation; (8) the first Fc domain comprises a D401K mutation and the second Fc domain comprises T411E/K360E/Q362E mutations; (9) the first Fc domain comprises L368D/K370S mutations and the second Fc domain comprises S364K/E357L mutations; (10) the first Fc domain comprises S364K/E357L mutations and the second Fc domain comprises L368D/K370S mutations; (11) the first Fc domain comprises a K370S mutation and the second Fc domain comprises S364K/E357Q mutations; (12) the first Fc domain comprises S364K/E357Q mutations and the second Fc domain comprises a K370S mutation; (13) the first Fc domain comprises T366S/L368A/Y407V mutations and the second Fc domain comprises a T366W mutation; (14) the first Fc domain comprises a T366W mutation and the second Fc domain comprises T366S/L368A/Y407V mutations; (15) the first Fc domain comprises T366S/L368A/Y407V/Y349C mutations and the second Fc domain comprises T366W/S354C mutations; or (16) the first Fc domain comprises T366W/S354C mutations and the second Fc domain comprises T366S/L368A/Y407V/Y349C mutations.

26. The antibody, or antigen-binding fragment thereof, of claim 20, wherein (1) the first Fc domain comprises F405A/Y407V mutations and the second Fc domain comprises a T394W mutation; (2) the first Fc domain comprises a T394W mutation and the second Fc domain comprises F405A/Y407V mutations; (3) the first Fc domain comprises F405A/Y407V mutations and the second Fc domain comprises T366I/T394W mutations; (4) the first Fc domain comprises T366I/T394W mutations and the second Fc domain comprises F405A/Y407V mutations; (5) the first Fc domain comprises F405A/Y407V mutations and the second Fc domain comprises T366L/T394W mutations; (6) the first Fc domain comprises T366L/T394W mutations and the second Fc domain comprises F405A/Y407V mutations; (7) the first Fc domain comprises F405A/Y407V mutations and the second Fc domain comprises T366L/K392M/T394W mutations; (8) the first Fc domain comprises T366L/K392M/T394W mutations and the second Fc domain comprises F405A/Y407V mutations; (9) the first Fc domain comprises L351Y/F405A/Y407V mutations and the second Fc domain comprises T366L/K329M/T394W mutations; (10) the first Fc domain comprises T366L/K329M-/T394W mutations and the second Fc domain comprises L351Y/F405A/Y407V mutations; (11) the first Fc domain comprises T350V/L351Y/F405A/Y407V mutations and the second Fc domain comprises T350V/T366L/K392M/T394W mutations; (12) the first Fc domain comprises T350V/T366L/K392M/T394W mutations and the second Fc domain comprises T350V/L351Y/F405A/Y407V mutations; (13) the first Fc domain comprises T350V/L351Y/F405A/Y407V mutations and the second Fc domain comprises T350V/T366L-/K392L/T394W mutations; or (14) the first Fc domain comprises T350V/T366L/K392L-/T394W mutations and the second Fc domain comprises T350V/L351Y/F405A/Y407V mutations.

27. The antibody, or antigen-binding fragment thereof, of claim 20, wherein (1) the first Fc domain comprises D399M/Y407A mutations and the second Fc domain comprises T366V/K409V mutations; (2) the first Fc domain comprises T366V/K409V mutations and the second Fc domain comprises D399M/Y407A mutations; (3) the first Fc domain comprises K360D/D399M/Y407A mutations and the second Fc domain comprises E345R/Q347R/T366V/K409V mutations; (4) the first Fc domain comprises E345R/Q347R/T366V/K409V mutations and the second Fc domain comprises K360D/D399M/Y407A mutations; (5) the first Fc domain comprises Y349S/K370Y/T366V-/K409V mutations and the second Fc domain comprises E357D/S364Q/Y407A mutations; (6) the first Fc domain comprises E357D/S364Q/Y407A mutations and the second Fc domain comprises Y349S/K370Y/T366V/K409V mutations; (7) the first Fc domain comprises Y349S/K370Y/T366M/K409V mutations, and the second Fc domain comprises E356G/E357D/S364Q/Y407A mutations; (8) the first Fc domain comprises E356G/E357D/S364Q/Y407A mutations, and the second Fc domain comprises Y349S/K370Y/T366M/K409V mutations; (9) the first Fc domain comprises Y349S/K370Y/T366M/K409V mutations and the second Fc domain comprises E357D/S364R/Y407A mutations; or (10) the first Fc domain comprises E357D/S364R/Y407A mutations and the second Fc domain comprises Y349S/K370Y/T366M/K409V mutations.

28. The antibody, or antigen-binding fragment thereof, of claim 20, wherein (1) the first Fc domain comprises K409D/K392D mutations and the second Fc domain comprises D399K/E356K mutations; (2) the first Fc domain comprises D399K/E356K mutations and the second Fc domain comprises K409D/K392D mutations; (3) the first Fc domain comprises K409E/K392D mutations and the second Fc domain comprises D399R/E356K mutations; (4) the first Fc domain comprises D399R/E356K mutations and the second Fc domain comprises K409E/K392D mutations; (5) the first Fc domain comprises K409D/K392D mutations and the second Fc domain comprises D399R/E356K mutations; (6) the first Fc domain comprises D399R/E356K mutations and the second Fc domain comprises K409D/K392D mutations; (7) the first Fc domain comprises K409E/K392D mutations and the second Fc domain comprises D399K/E356K mutations; (8) the first Fc domain comprises D399K/E356K mutations and the second Fc domain comprises K409E/K392D mutations; (9) the first Fc domain comprises K409D/K392D/K370D mutations and the second Fc domain comprises D399K/E356K/-E357K mutations; or (10) the first Fc domain comprises D399K/E356K/E357K mutations and the second Fc domain comprises K409D/K392D/K370D mutations.

29. The antibody, or antigen-binding fragment thereof, of claim 20, wherein (1) the first Fc domain comprises a K409W mutation and the second Fc domain comprises F405T/D399V mutations; (2) the first Fc domain comprises F405T/D399V mutations and the second Fc domain comprises a K409W mutation; (3) the first Fc domain comprises K360E/K409W mutations and the second Fc domain comprises Q347R/D399V/F405T mutations; (4) the first Fc domain comprises Q347R/D399V/F405T mutations and the second Fc domain comprises K360E/K409W mutations; (5) the first Fc domain comprises K360E/K409W/Y349C mutations and the second Fc domain comprises Q347R/D399V/F405T/S354C mutations; or (6) the first Fc domain comprises Q347R/D399V/F405T/S354C mutations and the second Fc domain comprises K360E/K409WY349C mutations.

30. The antibody, or antigen-binding fragment thereof, of claim 20, wherein (1) the first Fc domain comprises an AG SEED domain and the second Fc domain comprises a GA SEED domain or (2) the first Fc domain comprises a GA SEED domain and the second Fc domain comprises an AG SEED domain.

31. The antibody, or antigen-binding fragment thereof, of claim 20, wherein (1) the first Fc domain comprises K370E/K409W mutations and the second Fc domain comprises E357N/D399V/F405T mutations; (2) the first Fc domain comprises E357N/D399V/F405T mutations and the second Fc domain comprises K370E/K409W mutations; (3) the first Fc domain comprises K370E/K409W mutations and the second Fc domain comprises E3571/S364T/D399V/F405T mutations; (4) the first Fc domain comprises E3571/S364T/D399V/F405T mutations and the second Fc domain comprises K370E/K409W mutations; (5) the first Fc domain comprises K370M/K409W mutations and the second Fc domain comprises E357M/S364W/D399V/F405T mutations; (6) the first Fc domain comprises E357M/S364W/D399V/F405T mutations and the second Fc domain comprises K370M/K409W mutations; (7) the first Fc domain comprises K370D/K409W mutations and the second Fc domain comprises E357M/D399V/F405T mutations; (8) the first Fc domain comprises E357M/D399V/F405T mutations and the second Fc domain comprises K370D/K409W mutations; (9) the first Fc domain comprises E357D/S364W/K370E mutations and the second Fc domain comprises E357N/K370R mutations; (10) the first Fc domain comprises E357N/K370R mutations and the second Fc domain comprises E357D/S364W/K370E mutations; (11) the first Fc domain comprises E357A/S364Y/K370E mutations and the second Fc domain comprises E357N/K370H mutations; (12) the first Fc domain comprises E357N/K370H mutations and the second Fc domain comprises E357A/S364Y/K370E mutations; (13) the first Fc domain comprises E357G/S364W/K370E mutations and the second Fc domain comprises an E357N mutation; (14) the first Fc domain comprises an E357N mutation and the second Fc domain comprises E357G/S364W/DK70E mutations; (15) the first Fc domain comprises E357N/S364W/K370E mutations and the second Fc domain comprises an E357N mutation; or (16) the first Fc domain comprises an E357N mutation and the second Fc domain comprises E357N/S364W/DK70E mutations.

32. The antibody, or antigen-binding fragment thereof, of any one of claims 8-31, wherein the first and/or second Fc domain are independently engineered to have improved pharmacokinetics (PK).

33. The antibody, or antigen-binding fragment thereof, of claim 32, wherein the first and/or second Fe domains comprise amino acid substitutions M428L and/or N434S.

34. The antibody, or antigen-binding fragment thereof, of claim 10, wherein the anti-tumor antibody is an anti-CD33 antibody.

35. The antibody, or antigen-binding fragment thereof, of claim 34, wherein the anti-CD33 antibody comprises the six CDRs of gemtuzumab.

36. The antibody, or antigen-binding fragment thereof, of claim 35, wherein the anti-CD33 antibody comprises the VH of gemtuzumab.

37. The antibody, or antigen-binding fragment thereof, of claim 35 or 36, wherein the anti-CD33 antibody comprises the VL of gemtuzumab.

38. The antibody, or antigen-binding fragment thereof, of claim 34, wherein the anti-CD33 antibody comprises the VH comprising the amino acid sequence of SEQ ID NO: 1151.

39. The antibody, or antigen-binding fragment thereof, of claim 34 or 38, wherein the anti-CD33 antibody comprises the VL comprising the amino acid sequence of SEQ ID NO:1152.

40. The antibody, or antigen-binding fragment thereof, of any one of claims 1-39, wherein the antibody is selected from an IgG antibody, an IgM antibody, an IgA antibody, an IgD antibody, and an IgE antibody.

41. The antibody, or antigen-binding fragment thereof, of claim 40, wherein the antibody is an IgG antibody.

42. The antibody, or antigen-binding fragment thereof, of claim 41, wherein the antibody is a variant of an IgG antibody selected from an IgG1 antibody, an IgG2 antibody, an IgG3 antibody or an IgG4 antibody.

43. The antibody, or antigen-binding fragment thereof, of claim 42, wherein the antibody comprises a modified Fc domain derived from an IgG1 molecule.

44. The antibody, or antigen-binding fragment thereof, of claim 43, wherein the modified Fc domain derived from the IgG1 molecule comprises an amino acid modification at any one of the positions selected from the group consisting of E216, R217, K218, C219, C220, C226, C229, P230, E233, L234, L235, G236, G237, P238, S239, V240, F241, K246, L251, T260, D265, V266, H268, W277, N297, E318, K322, P329, A330, P331, Q347, N348, T350, L351, K360, T366, N390, K392, T394, D399, S400, F405, Y407, K409, T411 or a combination such amino acid modifications.

45. The antibody, or antigen-binding fragment thereof, of claim 43, wherein the modified Fc domain derived from IgG1 comprises an amino acid modification selected from the group consisting of C220S, C226S, C229S, P230S, E233P, L234A, L234F, L234V, L235A, L235E, L235V, G236E, G237A, P238S, D265S, D265A, H268Q, W277T, N297G, N297Q, N297D, N297A, E318A, K322A, P329G, P329A, A330S, P331S, Q347R, Q347E, Q347K, T350V, L351Y, K360D, K360E, T366A, T366I, T366L, T366M, T366V, N390R, N390K, N390D, K392V, K392M, K392R, K392L, K392F, K392E, T394W, D399R, D399W, D399K, S400E, S400D, S400R, S400K, F405A, F405I, F405M, F405T, F405S, F405V, F405W, Y407A, Y407I, Y407L, Y407V, K409F, K409I, K409S, K409W, T411N, T411R, T411Q, T411K, T411D, T411E, T411W, ΔE216-E222, K246R/L251E/T260R, InR234/235, InV235/236, InR236/237, InR237/238, InV238/239, InN238/239, InL238/239, InE238/239, InG238/239, InS239/240, InG240/241, InE240/241, InG240/241, InL238/239/P238Q, InE238/239/N348A, InS239/240/V266A, and InR237/238/G236A or a combination thereof.

46. The antibody, or antigen-binding fragment thereof, of claim 45, wherein the modified Fc domain derived from IgG1 comprises L234F/L235E/P331S mutations.

47. The antibody, or antigen-binding fragment thereof, of claim 42, wherein the antibody comprises a modified Fc domain derived from an IgG2 molecule.

48. The antibody, or antigen-binding fragment thereof, of claim 47, wherein the modified Fc domain derived from the IgG2 molecule comprises an amino acid modification at any one of the positions selected from the group consisting of V234, G237, P238, H268, V309, A330, and P331 or a combination thereof.

49. The antibody, or antigen-binding fragment thereof, of claim 48, wherein the modified Fc domain derived from the IgG2 molecule comprises an amino acid modification at any one of the positions selected from the group consisting of V234A, G237A, P238S, H268Q, H268A, V309L, A330S, P331S, or a combination thereof.

50. The antibody, or antigen-binding fragment thereof, of claim 42, wherein the antibody comprises a modified Fc domain derived from an IgG4 molecule.

51. The antibody, or antigen-binding fragment thereof, of claim 50, wherein the modified Fc domain derived from the IgG4 molecule comprises an amino acid modification at any one of the positions selected from the group consisting of S228, L235, L236, G237, E318, and N297 or a combination thereof.

52. The antibody, or antigen-binding fragment thereof, of claim 51, wherein the modified Fc domain derived from the IgG4 molecule comprises an amino acid modification at any one of the positions selected from the group consisting of S228P, L235A, L235E, L236E, G237A, E318A, and N297Q or a combination thereof.

53. A recombinant polynucleotide encoding an anti-CD3 antibody, or antigen-binding fragment thereof, of any one of claims 1-52.

54. An expression vector comprising the recombinant polynucleotide of claim 53.

55. A first recombinant polynucleotide encoding the heavy chain of an anti-CD3 antibody, or antigen-binding fragment thereof, of any one of claims 1-52 and a second recombinant polynucleotide encoding the light chain of an anti-CD3 antibody, or antigen-binding fragment thereof, of any one of claims 1-52.

56. A first expression vector comprising the first recombinant polynucleotide of claim 55 and a second expression vector comprising the second recombinant polynucleotide of claim 55.

57. An isolated host cell comprising the recombinant polynucleotide of claim 53, the expression vector of claim 54, the first and second recombinant polynucleotides of claim 55 or the first and second expression vectors of claim 56.

58. A method of producing the antibody, or antigen-binding fragment thereof, or any one of claims 1-52, the method comprising culturing the host cell of claim 57 under conditions suitable for expressing the antibody or fragment.

59. A composition comprising the antibody, or antigen-binding fragment thereof, of any one of claims 1-52 and a physiologically acceptable carrier.

60. A method of treating or preventing cancer in a subject in need thereof, the method comprising administering the antibody, or antigen-binding fragment thereof, of any one of claims 1-52 or the composition of claim 59 to the subject in need thereof.

61. A method of maintaining cancer remission in a subject, the method comprising administering the antibody, or antigen-binding fragment thereof, of any one of claims 1-52 or the composition of claim 59 to the subject in need thereof.

62. A method of targeting a T-cell to a tumor cell, wherein the method comprises contacting the T-cell with the antibody, or antigen-binding fragment thereof, any one of claims 8-52 or the composition of claim 59.

63. The method of any one of claims 60-62, wherein the anti-CD3 antibody is an anti-CD3 bispecific antibody, wherein the bispecific antibody further comprises an anti-tumor antibody.

64. A method of treating or preventing an autoimmune disease or disorder in a subject in need thereof, the method comprising administering the antibody, or antigen-binding fragment thereof, of any one of claims 1-52 or the composition of claim 59 to the subject in need thereof

65. The method of claim 64, wherein the autoimmune disease or disorder is graft-versus-host disease or type I diabetes.
